(19) United States
(12) Patent Application Publication
(10) Pub. No.: US 2025/0101041 A1
(43) Pub. Date: Mar. 27, 2025

(54) POSITIVE ALLOSTERIC MODULATORS OF THE MUSCARINIC ACETYLCHOLINE RECEPTOR M4

(71) Applicant: Vanderbilt University, Nashville, TN (US)

(72) Inventors: Craig W. Lindsley, Brentwood, TN (US); P. Jeffrey Conn, Nashville, TN (US); Darren W. Engers, Brentwood, TN (US); Kayla J. Temple, Spring Hill, TN (US); Alison R. Gregro, Mount Juliet, TN (US); Madeline F. Long, Nashville, TN (US); Logan A. Baker, Thompson's Station, TN (US); Aaron M. Bender, Spring Hill, TN (US); Anna E. Ringuette, Nashville, TN (US); Sara L. Biscotto, Nashville, TN (US)

(21) Appl. No.: 18/729,768

(22) PCT Filed: Jan. 19, 2023
(86) PCT No.: PCT/US23/60913

§ 371 Date: Jul. 17, 2024

Related U.S. Application Data
(60) Provisional application No. 63/300,715, filed on Jan. 19, 2022.

(51) Int. Cl. C07D 519/00; A61K 31/519; A61K 31/5377; A61K 31/5383; C07D 487/04

(52) U.S. Cl. CPC C07D 519/00; A61K 31/519; A61K 31/5377; A61K 31/5383; C07D 487/04

(57) ABSTRACT

5,6,7,8-Tetrahydro-1,6-naphthyridines substituted in the 6-position with pyrimido[1,2-b]pyridazin-4-one are positive allosteric modulators of the muscarinic acetylcholine receptor M₄ (mAChR M₄) and may have use in treating neurological and psychiatric disorders associated with muscarinic acetylcholine receptor dysfunction.

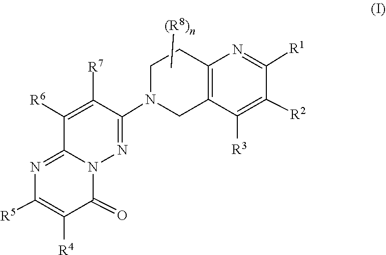

### RELATED APPLICATIONS

This application claims priority to U.S. Provisional Application No. 63/300,715, filed Jan. 19, 2022, which is hereby incorporated by reference in its entirety.

### TECHNICAL FIELD

The present disclosure relates to compounds, compositions, and methods for treating neurological and psychiatric disorders associated with muscarinic acetylcholine receptor dysfunction.

### BACKGROUND

Cholinergic neurotransmission involves the activation of nicotinic acetylcholine receptors (nAChRs) or the muscarinic acetylcholine receptors (mAChRs) by the binding of the endogenous orthosteric agonist acetylcholine (ACh). Conditions associated with cognitive impairment, such as Alzheimer's disease, are accompanied by a reduction of acetylcholine content in the brain. This is believed to be the result of degeneration of cholinergic neurons of the basal forebrain, which widely innervate multiple areas of the brain, including the association cortices and hippocampus, which are critically involved in higher processes. Clinical data supports that cholinergic hypofunction contributes to the cognitive deficits of patients suffering from schizophrenia. Efforts to increase acetylcholine levels have focused on increasing levels of choline, the precursor for acetylcholine synthesis, and on blocking acetylcholinesterase (AChE), the enzyme that metabolizes acetylcholine. As a result, acetylcholinesterase (AChE) inhibitors, which inhibit the hydrolysis of ACh, have been approved in the United States for use in the palliative, but not disease-modifying, treatment of the cognitive deficits in AD patients.

Attempts to augment central cholinergic function through the administration of choline orphosphatidylcholine have not been successful. AChE inhibitors have shown therapeutic efficacy, but have been found to have frequent cholinergic side effects due to peripheral acetylcholine stimulation, including abdominal cramps, nausea, vomiting, and diarrhea. These gastrointestinal side effects have been observed in about a third of the patients treated. In addition, some AChE inhibitors, such as tacrine, have also been found to cause significant hepatotoxicity with elevated liver transaminases observed in about 30% of patients. The adverse effects of AChE inhibitors have severely limited their clinical utility. An alternative approach to pharmacologically target cholinergic hypofunction is the activation of mAChRs, which are widely expressed throughout the body.

The mAChRs are members of the family A Gprotein-coupled receptors (GPCRs) and include five subtypes, designated M₁-M₅. The M₁, M₃ and M₅ subtypes mainly couple to G_{q} and activate phospholipase C, whereas the M₂ and M₄ subtypes mainly couple to G_{i/o} and associated effector systems. These five distinct mAChR subtypes have been identified in the mammalian central nervous system where they are prevalent and differentially expressed. M₁-M₅ have varying roles in cognitive, sensory, motor and autonomic functions. Thus, without wishing to be bound by a particular theory, it is believed that selective agonists of mAChR subtypes that regulate processes involved in cognitive function could prove to be superior therapeutics for treatment of psychosis, schizophrenia and related disorders. The muscarinic M₄ receptor has been shown to have a major role in cognitive processing and is believed to have a major role in the pathophysiology of psychotic disorders, including schizophrenia.

Evidence suggests that the most prominent adverse effects of AChE inhibitors and other cholinergic agents are mediated by activation of peripheral M₂ and M₃ mAChRs and include bradycardia, GI distress, excessive salivation, and sweating. In contrast, M₄ has been viewed as the most likely subtype for mediating the effects of muscarinic acetylcholine receptor dysfunction in psychotic disorders, including schizophrenia, cognition disorders, and neuropathic pain. Because of this, considerable effort has been focused on developing selective M₄ agonists for treatment of these disorders. Unfortunately, these efforts have been largely unsuccessful because of an inability to develop compounds that are highly selective for the mAChR M₄. Because of this, mAChR agonists that have been tested in clinical studies induce a range of adverse effects by activation of peripheral mAChRs. To fully understand the physiological roles of individual mAChR subtypes and to further explore the therapeutic utility of mAChR ligands in psychosis, including schizophrenia, cognition disorders and other disorders, it can be important to develop compounds that are highly selective activators of mAChR M₄ and other individual mAChR subtypes.

Previous attempts to develop agonists that are highly selective for individual mAChR subtypes have failed because of the high conservation of the orthosteric ACh binding site. To circumvent problems associated with targeting the highly conserved orthosteric ACh binding site, it is believed that developing compounds that act at allosteric sites on mAChRs that are removed from the orthosteric site and are less highly conserved. This approach is proving to be highly successful in developing selective ligands for multiple GPCR subtypes. In the case of mAChRs, a major goal has been to develop allosteric ligands that selectively increase activity of mAChR M₄ or other mAChR subtypes. Allosteric activators can include allosteric agonists, that act at a site removed from the orthosteric site to directly activate the receptor in the absence of ACh as well as positive allosteric modulators (PAMs), which do not activate the receptor directly but potentiate activation of the receptor by the endogenous orthosteric agonist ACh. Also, it is possible for a single molecule to have both allosteric potentiator and allosteric agonist activity.

More recently, muscarinic agonists including xanomeline have been shown to be active in animal models with similar profiles to known antipsychotic drugs, but without causing catalepsy (Bymaster et al., Eur. J. Pharmacol. 1998, 356, 109, Bymaster et al., Life Sci. 1999, 64, 527; Shannon et al., J. Pharmacol. Exp. Ther. 1999, 290, 901; Shannon et al., Schizophrenia Res. 2000, 42, 249). Further, xanomeline was shown to reduce psychotic behavioral symptoms such as delusions, suspiciousness, vocal outbursts, and hallucinations in Alzheimer's disease patients (Bodick et al., Arch. Neurol. 1997, 54, 465), however treatment induced side effects, e.g., gastrointestinal effects, have severely limited the clinical utility of this compound.

Despite advances in muscarinic acetylcholine receptor research, there is still a scarcity of compounds that are potent, efficacious, and selective activators of the M₄ mAChR and also effective in the treatment of neurological and psychiatric disorders associated with cholinergic activity and diseases in which the muscarinic M₄ receptor is involved.

### SUMMARY

In one aspect, disclosed are compounds of formula (I), or a pharmaceutically acceptable salt thereof

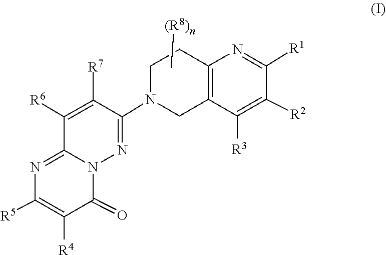

wherein:
R¹ and R³ are each independently hydrogen, halogen, cyano, C₁₋₄alkyl, C₁₋₄fluoroalkyl, —OC₁₋₄ alkyl, or —OC₁₋₄fluoroalkyl;
R² is hydrogen, halogen, cyano, NO₂, C₁₋₆alkyl, C₁₋₆haloalkyl, —OR^{b}, —NR^{b}R^{c}, —NR^{c}C(O)R^{b}, —NR^{c}SO₂R^{a}, —N═S(O)(R^{a})₂, —P(O)(R^{a})₂, G², or —C₁₋₃alkylene-G²;
R^{a}, at each occurrence, is independently C₁₋₆alkyl, C₁₋₆haloalkyl, G², or —C₁₋₃alkylene-G²; wherein optionally, the two R^{a} of —N═S(O)(R^{a})₂ or —P(O)(R^{a})₂ join together as a straight alkylene chain to form a 5- to 7-membered heterocyclic ring;
R^{b} and R^{c} are independently hydrogen, C₁₋₆alkyl, C₁₋₆haloalkyl, G², or —C₁₋₃alkylene-G²;
G², at each occurrence, is independently a 6- to 12-membered aryl, a 5- to 12-membered heteroaryl, a 4- to 12-membered heterocyclyl, or a 3- to 12-membered carbocyclyl, wherein the heteroaryl and heterocyclyl each contain 1-4 heteroatoms independently selected from the group consisting of O, N, and S, and G² is optionally substituted with 1-5 substituents independently selected from the group consisting of halogen, cyano, C₁₋₆alkyl, C₁₋₆haloalkyl, oxo, —OR^{x}, —N(R^{x})₂, —C(O)R^{x}, —C(O)OR^{x}, —C(O)N(R^{x})₂, —C₁₋₆alkylene-OR^{x}, —C₁₋₆alkylene-N(R^{x})₂, G^{2a}, and —C₁₋₃alkylene-G^{2a};
R^{x}, at each occurrence, is independently hydrogen, C₁₋₄alkyl, C₁₋₄haloalkyl, C₃₋₆cycloalkyl, or —C₁₋₃alkylene-C₃₋₆cycloalkyl, wherein each cycloalkyl is optionally substituted with 1-4 substituents independently selected from the group consisting of halogen, C₁₋₄alkyl, and C₁₋₄ haloalkyl;
G^{2a} is a phenyl, a 5- to 6-membered heteroaryl containing 1-3 heteroatoms, a 4- to 8-membered heterocyclyl containing 1-2 heteroatoms, or a 3- to 8-membered carbocyclyl, wherein the heteroatoms are independently selected from the group consisting of O, N, and S, and G^{2a}, at each occurrence, is independently optionally substituted with 1-5 substituents independently selected from the group consisting of halogen, cyano, C₁₋₄alkyl, C₁₋₄haloalkyl, —C₁₋₆ alkylene-OH, oxo, OH, —OC₁₋₄alkyl, —OC₁₋₄haloalkyl, C₃₋₄cycloalkyl, and —C₁₋₃alkylene-C₃₋₄cycloalkyl;
R⁴ is hydrogen, halogen, C₁₋₄alkyl, C₁₋₄fluoroalkyl, C₂₋₄alkenyl, —N(R^{4a})₂, or G⁴;
R^{4a}, at each occurrence, is independently hydrogen, C₁₋₄alkyl, C₁₋₄fluoroalkyl, C₃₋₄cycloalkyl, or —C₁₋₃alkylene-C₃₋₄cycloalkyl;
G⁴ is a phenyl, a 5- to 6-membered heteroaryl containing 1-3 heteroatoms, a 4- to 8-membered heterocyclyl containing 1-2 heteroatoms, or a 3- to 6-membered carbocyclyl, wherein the heteroatoms are independently selected from the group consisting of O, N, and S, and G⁴ is optionally substituted with 1-5 substituents independently selected from the group consisting of halogen, cyano, C₁₋₄alkyl, C₁₋₄fluoroalkyl, oxo, OH, —OC₁₋₄alkyl, C₃₋₄cycloalkyl, and —C₁₋₃ alkylene-C₃₋₄cycloalkyl;
R⁵ is hydrogen, C₁₋₄alkyl, C₁₋₄fluoroalkyl, —C₁₋₃alkylene-OR^{5a}, —C(O)NR^{5a}R^{5b}, C₃₋₆cycloalkyl, or —C₁₋₃alkylene-C₃₋₆cycloalkyl;
R^{5a} is hydrogen, C₁₋₄alkyl, C₁₋₄fluoroalkyl, C₃₋₄cycloalkyl, or —C₁₋₃alkylene-C₃₋₄cycloalkyl;
R^{5b} is hydrogen, C₁₋₆alkyl, C₁₋₆fluoroalkyl, —C₁₋₆alkylene-OH, —C₁₋₆alkylene-OC₁₋₄alkyl, G⁵, or —C₁₋₃alkylene-G⁵;
wherein alternatively, R^{5a} and R^{5b}, together with the nitrogen to which they attach form a 4- to 10-membered heterocyclic ring containing the nitrogen attached to R^{5a} and R^{5b} and optionally 1-2 additional heteroatoms that are independently O, N, or S, the heterocyclic ring being optionally substituted with 1-4 substituents independently selected from the group consisting of halogen, cyano, C₁₋₄alkyl, C₁₋₄fluoroalkyl, —C₁₋₆alkylene-OH, oxo, —OR⁵⁰, —N(R⁵⁰)₂, —NR⁵⁰C(O)R⁵⁰, —NR⁵⁰SO₂R⁵⁰, —C(O)OR⁵O, —C(O)N(R⁵⁰)₂, —SO₂R⁵⁰, G^{5a}, and —C₁₋₃ alkylene-G^{5a};
G⁵ is a phenyl, a 5- to 6-membered heteroaryl containing 1-3 heteroatoms, a 4- to 8-membered heterocyclyl containing 1-2 heteroatoms, or a C₃₋₆cycloalkyl, wherein the heteroatoms are independently selected from the group consisting of O, N, and S, and G⁵ is optionally substituted with 1-5 substituents independently selected from the group consisting of halogen, cyano, C₁₋₄alkyl, C₁₋₄fluoroalkyl, —C₁₋₆alkylene-OH, oxo, —OR⁵⁰, —N(R⁵⁰)₂, —NR⁵⁰C(O)R⁵⁰, —NR⁵⁰SO₂R⁵⁰, —C(O)OR⁵⁰, —C(O)N(R⁵⁰)₂, —SO₂R⁵⁰, G^{5a}, and —C₁₋₃alkylene-G^{5a}.
R⁵⁰, at each occurrence, is independently hydrogen, C₁₋₄alkyl, C₁₋₄fluoroalkyl, C₃₋₆cycloalkyl, or —C₁₋₃alkylene-C₃₋₆cycloalkyl, wherein each cycloalkyl is optionally substituted with 1-4 substituents independently selected from the group consisting of halogen, C₁₋₄alkyl, and C₁₋₄ fluoroalkyl;
G^{5a}, at each occurrence, is independently a phenyl, a 5- to 6-membered heteroaryl containing 1-3 heteroatoms, a 4- to 8-membered heterocyclyl containing 1-2 heteroatoms, or a 3- to 8-membered carbocyclyl, wherein the heteroatoms are independently selected from the group consisting of O, N, and S, and G^{5a} is optionally substituted with 1-5 substituents independently selected from the group consisting of halogen, cyano, C₁₋₄alkyl, C₁₋₄fluoroalkyl, oxo, OH, —OC₁₋₄alkyl, —OC₁₋₄fluoroalkyl, C₃₋₄cycloalkyl, and —C₁₋₃alkylene-C₃₋₄cycloalkyl;
R⁶ is hydrogen, halogen, C₁₋₄alkyl, C₁₋₄fluoroalkyl, —OR^{6a}, —N(R^{6a})₂, —C₁₋₃alkylene-OR^{6a}, or C₃₋₆ cycloalkyl;
R^{6a}, at each occurrence, is independently hydrogen, C₁₋₄alkyl, C₁₋₄fluoroalkyl, C₃₋₄cycloalkyl, or —C₁₋₃alkylene-C₃₋₄cycloalkyl;
wherein alternatively, two R^{6a}, together with the nitrogen to which they attach form a 4- to 8-membered heterocyclic ring containing the nitrogen attached to R^{6a} and optionally 1-2 additional heteroatoms that are independently O, N, or S, the heterocyclic ring being optionally substituted with 1-4 substituents independently selected from the group consisting of halogen, C₁₋₂alkyl, and C₁₋₂fluoroalkyl;
R⁷ is hydrogen, C₁₋₄alkyl, C₁₋₄fluoroalkyl, —OR^{7a}, —C₁₋₃alkylene-OR^{7a}, or C₃₋₆cycloalkyl;
R^{7a} is hydrogen, C₁₋₄alkyl, C₁₋₄fluoroalkyl, C₃₋₄cycloalkyl, or —C₁₋₃alkylene-C₃₋₄cycloalkyl;
R⁸, at each occurrence, is independently halogen, C₁₋₄alkyl, C₁₋₄fluoroalkyl, or C₃₋₄cycloalkyl; and
n is 0, 1, 2, 3, or 4.

In another aspect, the invention provides a pharmaceutical composition comprising a compound of formula (I), or a pharmaceutically acceptable salt thereof, and a pharmaceutically acceptable carrier.

Another aspect provides a method of treating a neurological and/or psychiatric disorder associated with muscarinic acetylcholine receptor dysfunction in a mammal, comprising administering to the mammal a therapeutically effective amount of the compound of formula (I), or pharmaceutically acceptable salt or composition thereof.

Another aspect provides a compound of formula (I), or a pharmaceutically acceptable salt or composition thereof, for use in the treatment of a neurological and/or psychiatric disorder associated with muscarinic acetylcholine receptor dysfunction in a mammal.

Another aspect provides use of a compound of formula (I), or a pharmaceutically acceptable salt or composition thereof, for the preparation of a medicament for the treatment of a neurological and/or psychiatric disorder associated with muscarinic acetylcholine receptor dysfunction in a mammal.

In another aspect, the invention provides kits comprising a compound of formula (I), or a pharmaceutically acceptable salt or composition thereof, and instructions for use.

### DETAILED DESCRIPTION

Disclosed herein are positive allosteric modulators (i.e. potentiators) of the muscarinic acetylcholine receptor M₄ (mAChR M₄), methods of making same, pharmaceutical compositions comprising same, and methods of treating neurological and psychiatric disorders associated with muscarinic acetylcholine receptor dysfunction using same. The compounds include naphthyridine-substituted pyridazine compounds.

The human muscarinic acetylcholine receptor M₄ (mAChR M₄) is a protein of 479 amino acids encoded by the CHRM₄ gene. The molecular weight of the unglycosylated protein is about 54 kDa and it is a transmembrane GPCR. As described above, the mAChR M₄ is a member of the GPCR Class A family, or the rhodopsin-like GPCRs, which are characterized by structural features similar to rhodopsin such as seven transmembrane segments. The muscarinic acetylcholine receptors have the N-terminus oriented to the extracellular face of the membrane and the C-terminus located on the cytoplasmic face.

Previous attempts to develop agonists that are highly selective for individual mAChR subtypes have failed because of the high conservation of the orthosteric ACh binding site. To circumvent problems associated with targeting the highly conserved orthosteric ACh binding site, it is believed that developing compounds that act at allosteric sites on mAChRs that are removed from the orthosteric site and are less highly-conserved. Without wishing to be bound by a particular theory, the disclosed compounds and products of the disclosed methods are believed to bind to an allosteric site distinct from the orthosteric binding site.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's AdvancedOrganic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkoxy,” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy and tert-butoxy.

The term “alkyl,” as used herein, means a straight or branched, saturated hydrocarbon chain. The term “lower alkyl” or “C₁₋₆alkyl” means a straight or branched chain hydrocarbon containing from 1 to 6 carbon atoms. The term “C₁₋₄alkyl” means a straight or branched chain saturated hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkenyl,” as used herein, means a straight or branched, hydrocarbon chain containing at least one carbon-carbon double bond.

The term “alkoxyalkyl,” as used herein, refers to an alkoxy group, as defined herein, appended to the parent molecular moiety through an alkyl group, as defined herein.

The term “alkoxyfluoroalkyl,” as used herein, refers to an alkoxy group, as defined herein, appended to the parent molecular moiety through a fluoroalkyl group, as defined herein.

The term “alkylene,” as used herein, refers to a divalent group derived from a straight or branched saturated chain hydrocarbon, for example, of 1 to 6 carbon atoms. Representative examples of alkylene include, but are not limited to, —CH₂—, —CH₂CH₂—, —CH₂CH₂CH₂—, —CH₂CH(CH₃)CH₂—, —CH₂CH₂CH₂CH₂—, —CH₂CH(CH₃)CH₂CH₂—, and —CH₂CH₂CH₂CH₂CH₂—.

The term “alkylamino,” as used herein, means at least one alkyl group, as defined herein, is appended to the parent molecular moiety through an amino group, as defined herein.

The term “amide,” as used herein, means —C(O)NR— or —NRC(O)—, wherein R may be hydrogen, alkyl, cycloalkyl, aryl, heteroaryl, heterocycle, alkenyl, or heteroalkyl.

The term “aminoalkyl,” as used herein, means at least one amino group, as defined herein, is appended to the parent molecular moiety through an alkylene group, as defined herein.

The term “amino,” as used herein, means —NR^{x}R_{y}, wherein R^{x} and R_{y} may be hydrogen, alkyl, cycloalkyl, aryl, heteroaryl, heterocycle, alkenyl, or heteroalkyl. In the case of an aminoalkyl group or any other moiety where amino appends together two other moieties, amino may be —NR^{x}—, wherein R^{x} may be hydrogen, alkyl, cycloalkyl, aryl, heteroaryl, heterocycle, alkenyl, or heteroalkyl.

The term “aryl,” as used herein, refers to a phenyl or a phenyl appended to the parent molecular moiety and fused to a cycloalkane group (e.g., the aryl may be indan-4-yl), fused to a 6-membered arene group (i.e., the aryl is naphthyl), or fused to a non-aromatic heterocycle (e.g., the aryl may be benzo[d][1,3]dioxol-5-yl). The term “phenyl” is used when referring to a substituent and the term 6-membered arene is used when referring to a fused ring. The 6-membered arene is monocyclic (e.g., benzene or benzo). The aryl may be monocyclic (phenyl) or bicyclic (e.g., a 9- to 12-membered fused bicyclic system).

The term “cyanoalkyl,” as used herein, means at least one —CN group, is appended to the parent molecular moiety through an alkylene group, as defined herein.

The term “cyanofluoroalkyl,” as used herein, means at least one —CN group, is appended to the parent molecular moiety through a fluoroalkyl group, as defined herein.

The term “cycloalkoxy,” as used herein, refers to a cycloalkyl group, as defined herein, appended to the parent molecular moiety through an oxygen atom.

The term “cycloalkyl” or “cycloalkane,” as used herein, refers to a saturated ring system containing all carbon atoms as ring members and zero double bonds. The term “cycloalkyl” is used herein to refer to a cycloalkane when present as a substituent. A cycloalkyl may be a monocyclic cycloalkyl (e.g., cyclopropyl), a fused bicyclic cycloalkyl (e.g., decahydronaphthalenyl), or a bridged cycloalkyl in which two non-adjacent atoms of a ring are linked by an alkylene bridge of 1, 2, 3, or 4 carbon atoms (e.g., bicyclo[2.2.1]heptanyl). Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, and bicyclo[1.1.1]pentanyl.

The term “cycloalkenyl” or “cycloalkene,” as used herein, means a non-aromatic monocyclic or multicyclic ring system containing all carbon atoms as ring members and at least one carbon-carbon double bond and preferably having from 5-10 carbon atoms per ring. The term “cycloalkenyl” is used herein to refer to a cycloalkene when present as a substituent. A cycloalkenyl may be a monocyclic cycloalkenyl (e.g., cyclopentenyl), a fused bicyclic cycloalkenyl (e.g., octahydronaphthalenyl), or a bridged cycloalkenyl in which two non-adjacent atoms of a ring are linked by an alkylene bridge of 1, 2, 3, or 4 carbon atoms (e.g., bicyclo[2.2.1]heptenyl). Exemplary monocyclic cycloalkenyl rings include cyclopentenyl, cyclohexenyl or cycloheptenyl. Exemplary monocyclic cycloalkenyl rings include cyclopentenyl, cyclohexenyl or cycloheptenyl.

The term “carbocyclyl” means a “cycloalkyl” or a “cycloalkenyl.” The term “carbocycle” means a “cycloalkane” or a “cycloalkene.” The term “carbocyclyl” refers to a “carbocycle” when present as a substituent.

The term “fluoroalkyl,” as used herein, means an alkyl group, as defined herein, in which one, two, three, four, five, six, seven or eight hydrogen atoms are replaced by fluorine. Representative examples of fluoroalkyl include, but are not limited to, 2-fluoroethyl, 2,2,2-trifluoroethyl, trifluoromethyl, difluoromethyl, pentafluoroethyl, and trifluoropropyl such as 3,3,3-trifluoropropyl.

The term “fluoroalkoxy,” as used herein, means at least one fluoroalkyl group, as defined herein, is appended to the parent molecular moiety through an oxygen atom. Representative examples of fluoroalkoxy include, but are not limited to, difluoromethoxy, trifluoromethoxy and 2,2,2-trifluoroethoxy.

The term “halogen” or “halo,” as used herein, means Cl, Br, I, or F.

The term “haloalkyl,” as used herein, means an alkyl group, as defined herein, in which one, two, three, four, five, six, seven or eight hydrogen atoms are replaced by a halogen.

The term “haloalkoxy,” as used herein, means at least one haloalkyl group, as defined herein, is appended to the parent molecular moiety through an oxygen atom.

The term “halocycloalkyl,” as used herein, means a cycloalkyl group, as defined herein, in which one or more hydrogen atoms are replaced by a halogen.

The term “heteroalkyl,” as used herein, means an alkyl group, as defined herein, in which one or more of the carbon atoms has been replaced by a heteroatom selected from S, O, P and N. Representative examples of heteroalkyls include, but are not limited to, alkyl ethers, secondary and tertiary alkyl amines, amides, and alkyl sulfides.

The term “heteroaryl,” as used herein, refers to an aromatic monocyclic heteroatom-containing ring (monocyclic heteroaryl) or a bicyclic ring system containing at least one monocyclic heteroaromatic ring (bicyclic heteroaryl). The term “heteroaryl” is used herein to refer to a heteroarene when present as a substituent. The monocyclic heteroaryl are five or six membered rings containing at least one heteroatom independently selected from the group consisting of N, O and S (e.g. 1, 2, 3, or 4 heteroatoms independently selected from O, S, and N). The five membered aromatic monocyclic rings have two double bonds and the six membered aromatic monocyclic rings have three double bonds. The bicyclic heteroaryl is an 8- to 12-membered ring system and includes a fused bicyclic heteroaromatic ring system (i.e., 10π electron system) such as a monocyclic heteroaryl ring fused to a 6-membered arene (e.g., quinolin-4-yl, indol-1-yl), a monocyclic heteroaryl ring fused to a monocyclic heteroarene (e.g., naphthyridinyl), and a phenyl fused to a monocyclic heteroarene (e.g., quinolin-5-yl, indol-4-yl). A bicyclic heteroaryl/heteroarene group includes a 9-membered fused bicyclic heteroaromatic ring system having four double bonds and at least one heteroatom contributing a lone electron pair to a fully aromatic 10π electron system, such as ring systems with a nitrogen atom at the ring junction (e.g., imidazopyridine) or a benzoxadiazolyl. A bicyclic heteroaryl also includes a fused bicyclic ring system composed of one heteroaromatic ring and one non-aromatic ring such as a monocyclic heteroaryl ring fused to a monocyclic carbocyclic ring (e.g., 6,7-dihydro-5H-cyclopenta[b]pyridinyl), or a monocyclic heteroaryl ring fused to a monocyclic heterocycle (e.g., 2,3-dihydrofuro[3,2-b]pyridinyl). The bicyclic heteroaryl is attached to the parent molecular moiety at an aromatic ring atom. Other representative examples of heteroaryl include, but are not limited to, indolyl (e.g., indol-1-yl, indol-2-yl, indol-4-yl), pyridinyl (includingpyridin-2-yl, pyridin-3-yl, pyridin-4-yl), pyrimidinyl, pyrazinyl, pyridazinyl, pyrazolyl (e.g., pyrazol-4-yl), pyrrolyl, benzopyrazolyl, 1,2,3-triazolyl (e.g., triazol-4-yl), 1,3,4-thiadiazolyl, 1,2,4-thiadiazolyl, 1,3,4-oxadiazolyl, 1,2,4-oxadiazolyl, imidazolyl, thiazolyl (e.g., thiazol-4-yl), isothiazolyl, thienyl, benzimidazolyl (e.g., benzimidazol-5-yl), benzothiazolyl, benzoxazolyl, benzoxadiazolyl, benzothienyl, benzofuranyl, isobenzofuranyl, furanyl, oxazolyl, isoxazolyl, purinyl, isoindolyl, quinoxalinyl, indazolyl (e.g., indazol-4-yl, indazol-5-yl), quinazolinyl, 1,2,4-triazinyl, 1,3,5-triazinyl, isoquinolinyl, quinolinyl, imidazo[1,2-a]pyridinyl (e.g., imidazo[1,2-a]pyridin-6-yl), naphthyridinyl, pyridoimidazolyl, thiazolo[5,4-b]pyridin-2-yl, and thiazolo[5,4-d]pyrimidin-2-yl.

The term “heterocycle” or “heterocyclic,” as used herein, means a monocyclic heterocycle, a bicyclic heterocycle, or a tricyclic heterocycle. The term “heterocyclyl” is used herein to refer to a heterocycle when present as a substituent. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocyclyls include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, 2-oxo-3-piperidinyl, 2-oxoazepan-3-yl, oxadiazolinyl, oxadiazolidinyl, oxazolinyl, oxazolidinyl, oxetanyl, oxepanyl, oxocanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a 6-membered arene, or a monocyclic heterocycle fused to a monocyclic cycloalkane, or a monocyclic heterocycle fused to a monocyclic cycloalkene, or a monocyclic heterocycle fused to a monocyclic heterocycle, or a monocyclic heterocycle fused to a monocyclic heteroarene, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. The bicyclic heterocyclyl is attached to the parent molecular moiety at a non-aromatic ring atom (e.g., indolin-1-yl). Representative examples of bicyclic heterocyclyls include, but are not limited to, chroman-4-yl, 2,3-dihydrobenzofuran-2-yl, 2,3-dihydrobenzothien-2-yl, 1,2,3,4-tetrahydroisoquinolin-2-yl, 2-azaspiro[3.3]heptan-2-yl, 2-oxa-6-azaspiro[3.3]heptan-6-yl, azabicyclo[2.2.1]heptyl (including 2-azabicyclo[2.2.1]hept-2-yl), azabicyclo[3.1.0]hexanyl (including 3-azabicyclo[3.1.0]hexan-3-yl), 2,3-dihydro-1H-indol-1-yl, isoindolin-2-yl, octahydrocyclopenta[c]pyrrolyl, octahydropyrrolopyridinyl, tetrahydroisoquinolinyl, 7-oxabicyclo[2.2.1]heptanyl, hexahydro-2H-cyclopenta[b]furanyl, 2-oxaspiro[3.3]heptanyl, 3-oxaspiro[5.5]undecanyl, 6-oxaspiro[2.5]octan-1-yl, and 3-oxabicyclo[3.1.0]hexan-6-yl. Tricyclic heterocycles are exemplified by a bicyclic heterocycle fused to a 6-membered arene, or a bicyclic heterocycle fused to a monocyclic cycloalkane, or a bicyclic heterocycle fused to a monocyclic cycloalkene, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxy pentalene, hexahydro-2H-2,5-methanocyclopenta[b]furan, hexahydro-1H-1,4-methanocyclopenta[c]furan, aza-adamantane(1-azatricyclo[3.3.1.13,7]decane), and oxa-adamantane (2-oxatricyclo[3.3.1.13,7]decane). The monocyclic, bicyclic, and tricyclic heterocyclyls are connected to the parent molecular moiety at a non-aromatic ring atom.

The term “hydroxyl” or “hydroxy,” as used herein, means an —OH group.

The term “hydroxyalkyl,” as used herein, means at least one —OH group, is appended to the parent molecular moiety through an alkylene group, as defined herein.

The term “hydroxyfluoroalkyl,” as used herein, means at least one —OH group, is appended to the parent molecular moiety through a fluoroalkyl group, as defined herein.

Terms such as “alkyl,” “cycloalkyl,” “alkylene,” etc. may be preceded by a designation indicating the number of atoms present in the group in a particular instance (e.g., “C₁₋₄alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

The terms “parent molecule” or “parent molecular moiety” refer to the entire portion of a molecule to which a substituent is attached, i.e., the remainder of the molecule.

The term “sulfonamide,” as used herein, means —S(O)₂NR^{z}— or —NR^{z}S(O)—, wherein R^{z} may be hydrogen, alkyl, cycloalkyl, aryl, heteroaryl, heterocycle, alkenyl, or heteroalkyl.

The term “substituents” refers to a group “substituted” on a group such as an alkyl, alkenyl, alkynyl, aryl, heteroaryl, cycloalkyl, heteroalkyl, or heterocycle group, at any atom of that group. Any atom can be substituted.

The term “substituted” refers to a group that may be further substituted with one or more non-hydrogen substituent groups. Substituent groups include, but are not limited to, halogen, =O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl. In some embodiments, a group is optionally substituted. In some embodiments, a group is optionally substituted with 1, 2, 3, 4, or 5 substituents. In some embodiments, an aryl, heteroaryl, cycloalkyl, or heterocycle is optionally substituted with 1, 2, 3, 4, or 5 substituents. In some embodiments, an aryl, heteroaryl, cycloalkyl, or heterocycle may be independently unsubstituted or substituted with 1, 2, or 3 substituents.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

The term “allosteric site” as used herein refers to a ligand binding site that is topographically distinct from the orthosteric binding site.

The term “modulator” as used herein refers to a molecular entity (e.g., but not limited to, a ligand and a disclosed compound) that modulates the activity of the target receptor protein.

The term “ligand” as used herein refers to a natural or synthetic molecular entity that is capable of associating or binding to a receptor to form a complex and mediate, prevent or modify a biological effect. Thus, the term “ligand” encompasses allosteric modulators, inhibitors, activators, agonists, antagonists, natural substrates and analogs of natural substrates.

The terms “natural ligand” and “endogenous ligand” as used herein are used interchangeably, and refer to a naturally occurring ligand, found in nature, which binds to a receptor.

The term “orthosteric site” as used herein refers to the primary binding site on a receptor that is recognized by the endogenous ligand or agonist for that receptor. For example, the orthosteric site in the mAChR M₄ receptor is the site that acetylcholine binds.

The term “mAChR M₄ receptor positive allosteric modulator” as used herein refers to any exogenously administered compound or agent that directly or indirectly augments the activity of the mAChR M₄ receptor in the presence or in the absence of acetylcholine, or another agonist, in an animal, in particular a mammal, for example a human. For example, a mAChR M₄ receptor positive allosteric modulator can increase the activity of the mAChR M₄ receptor in a cell in the presence of extracellular acetylcholine. The cell can be Chinese hamster ovary (CHO-K1) cells transfected with human mAChR M₄. The cell can be Chinese hamster ovary (CHO-K1) cells transfected with rat mAChR M₄ receptor. The cell can be Chinese hamster ovary (CHO-K1) cells transfected with a mammalian mAChR M₄. The term “mAChR M₄ receptor positive allosteric modulator” includes a compound that is a “mAChRM₄ receptor allosteric potentiator” or a “mAChR M₄ receptor allosteric agonist,” as well as a compound that has mixed activity comprising pharmacology of both an “mAChR M₄ receptor allosteric potentiator” and an “mAChR M₄ receptor allosteric agonist.” The term “mAChR M₄ receptor positive allosteric modulator also includes a compound that is a “mAChR M₄ receptor allosteric enhancer.”

The term “mAChR M₄ receptor allosteric potentiator” as used herein refers to any exogenously administered compound or agent that directly or indirectly augments the response produced by the endogenous ligand (such as acetylcholine) when the endogenous ligand binds to the orthosteric site of the mAChR M₄ receptor in an animal, in particular a mammal, for example a human. The mAChR M₄ receptor allosteric potentiator binds to a site other than the orthosteric site, that is, an allosteric site, and positively augments the response of the receptor to an agonist or the endogenous ligand. In some embodiments, an allosteric potentiator does not induce desensitization of the receptor, activity of a compound as an mAChR M₄ receptor allosteric potentiator provides advantages over the use of a pure mAChR M₄ receptor orthosteric agonist. Such advantages can include, for example, increased safety margin, higher tolerability, diminished potential for abuse, and reduced toxicity.

The term “mAChR M₄ receptor allosteric enhancer” as used herein refers to any exogenously administered compound or agent that directly or indirectly augments the response produced by the endogenous ligand (such as acetylcholine) in an animal, in particular a mammal, for example a human. In some embodiments, the allosteric enhancer increases the affinity of the natural ligand or agonist for the orthosteric site. In some embodiments, an allosteric enhancer increases the agonist efficacy. The mAChR M₄ receptor allosteric enhancer binds to a site other than the orthosteric site, that is, an allosteric site, and positively augments the response of the receptor to an agonist or the endogenous ligand. An allosteric enhancer has no effect on the receptor by itself and requires the presence of an agonist or the natural ligand to realize a receptor effect.

The term “mAChR M₄ receptor allosteric agonist” as used herein refers to any exogenously administered compound or agent that directly activates the activity of the mAChR M₄ receptor in the absence of the endogenous ligand (such as acetylcholine) in an animal, in particular a mammal, for example a human. The mAChR M₄ receptor allosteric agonist binds to a site that is distinct from the orthosteric acetylcholine site of the mAChRM₄ receptor. Because it does not require the presence of the endogenous ligand, activity of a compound as an mAChR M₄ receptor allosteric agonist provides advantages if cholinergic tone at a given synapse is low.

The term “mAChR M₄ receptor neutral allosteric ligand” as used herein refers to any exogenously administered compound or agent that binds to an allosteric site without affecting the binding or function of agonists or the natural ligand at the orthosteric site in an animal, in particular a mammal, for example a human. However, a neutral allostericligand can block the action of other allosteric modulators that act via the same site.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. COMPOUNDS

In one aspect, the invention provides compounds of formula (I), wherein R¹-R⁸ and n are as defined herein.

Unsubstituted or substituted rings (i.e., optionally substituted) such as aryl, heteroaryl, etc. are composed of both a ring system and the ring system's optional substituents. Accordingly, the ring system may be defined independently of its substituents, such that redefining only the ring system leaves any previous optional substituents present. For example, a 5- to 12-membered heteroaryl with optional substituents may be further defined by specifying the ring system of the 5- to 12-membered heteroaryl is a 5- to 6-membered heteroaryl (i.e., 5- to 6-membered heteroaryl ring system), in which case the optional substituents of the 5- to 12-membered heteroaryl are still present on the 5- to 6-membered heteroaryl, unless otherwise expressly indicated.

Where heterocyclic and heteroaromatic ring systems are defined to “contain” or as “containing” specified heteroatoms (e.g., 1-3 heteroatoms independently selected from the group consisting of O, N, and S), any ring atoms of the heterocyclic and heteroaromatic ring systems that are not one of the specified heteroatoms are carbon atoms.

In the following, numbered embodiments of the invention are disclosed. The first embodiment is denoted E1, subsequent embodiments are denoted E1.1, E1.2, E2, E2.1, E2.2, E3, E4, E5, E5.1, and so forth.

E1. A compound of formula (I), or a pharmaceutically acceptable salt thereof,

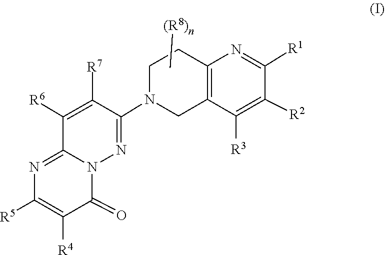

wherein:
R¹ and R³ are each independently hydrogen, halogen, cyano, C₁₋₄alkyl, C₁₋₄fluoroalkyl, —OC₁₋₄ alkyl, or —OC₁₋₄fluoroalkyl;
R² is hydrogen, halogen, cyano, NO₂, C₁₋₆alkyl, C₁₋₆haloalkyl, —OR^{b}, —NR^{b}R^{c}, —NR^{c}C(O)R^{b}, —NR^{c}SO₂R^{a}, —N═S(O)(R^{a})₂, —P(O)(R^{a})₂, G², or —C₁₋₃alkylene-G²;
R^{a}, at each occurrence, is independently C₁₋₆alkyl, C₁₋₆haloalkyl, G², or —C₁₋₃alkylene-G²;
wherein optionally, the two R^{a} of —N═S(O)(R^{a})₂ or —P(O)(R^{a})₂ join together as a straight alkylene chain to form a 5- to 7-membered heterocyclic ring;
R^{b} and R^{c} are independently hydrogen, C₁₋₆alkyl, C₁₋₆haloalkyl, G², or —C₁₋₃alkylene-G²;
G², at each occurrence, is independently a 6- to 12-membered aryl, a 5- to 12-membered heteroaryl, a 4- to 12-membered heterocyclyl, or a 3- to 12-membered carbocyclyl, wherein the heteroaryl and heterocyclyl each contain 1-4 heteroatoms independently selected from the group consisting of O, N, and S, and G² is optionally substituted with 1-5 substituents independently selected from the group consisting of halogen, cyano, C₁₋₆alkyl, C₁₋₆haloalkyl, oxo, —OR^{x}, —N(R^{x})₂, —C(O)R^{x}, —C(O)OR^{x}, —C(O)N(R^{x})₂, —C₁₋₆alkylene-OR^{x}, —C₁₋₆alkylene-N(R^{x})₂, G^{2a}, and —C₁₋₃alkylene-G^{2a};
R^{x}, at each occurrence, is independently hydrogen, C₁₋₄alkyl, C₁₋₄haloalkyl, C₃₋₆cycloalkyl, or —C₁₋₃alkylene-C₃₋₆cycloalkyl, wherein each cycloalkyl is optionally substituted with 1-4 substituents independently selected from the group consisting of halogen, C₁₋₄alkyl, and C₁₋₄ haloalkyl;
G^{2a} is a phenyl, a 5- to 6-membered heteroaryl containing 1-3 heteroatoms, a 4- to 8-membered heterocyclyl containing 1-2 heteroatoms, or a 3- to 8-membered carbocyclyl, wherein the heteroatoms are independently selected from the group consisting of O, N, and S, and G^{2a}, at each occurrence, is independently optionally substituted with 1-5 substituents independently selected from the group consisting of halogen, cyano, C₁₋₄alkyl, C₁₋₄haloalkyl, —C₁₋₆ alkylene-OH, oxo, OH, —OC₁₋₄alkyl, —OC₁₋₄haloalkyl, C₃₋₄cycloalkyl, and —C₁₋₃alkylene-C₃₋₄ cycloalkyl;
R⁴ is hydrogen, halogen, C₁₋₄alkyl, C₁₋₄fluoroalkyl, C₂₋₄alkenyl, —N(R^{4a})₂, or G⁴;
R^{4a}, at each occurrence, is independently hydrogen, C₁₋₄alkyl, C₁₋₄fluoroalkyl, C₃₋₄cycloalkyl, or —C₁₋₃alkylene-C₃₋₄cycloalkyl;
G⁴ is a phenyl, a 5- to 6-membered heteroaryl containing 1-3 heteroatoms, a 4- to 8-membered heterocyclyl containing 1-2 heteroatoms, or a 3- to 6-membered carbocyclyl, wherein the heteroatoms are independently selected from the group consisting of O, N, and S, and G⁴ is optionally substituted with 1-5 substituents independently selected from the group consisting of halogen, cyano, C₁₋₄alkyl, C₁₋₄fluoroalkyl, oxo, OH, —OC₁₋₄alkyl, C₃₋₄cycloalkyl, and —C₁₋₃ alkylene-C₃₋₄cycloalkyl;
R⁵ is hydrogen, C₁₋₄alkyl, C₁₋₄fluoroalkyl, —C₁₋₃alkylene-OR^{5a}, —C(O)NR^{5a}R^{5b}, C₃₋₆cycloalkyl, or —C₁₋₃alkylene-C₃₋₆cycloalkyl;
R^{5a} is hydrogen, C₁₋₄alkyl, C₁₋₄fluoroalkyl, C₃₋₄cycloalkyl, or —C₁₋₃alkylene-C₃₋₄cycloalkyl;
R^{5b} is hydrogen, C₁₋₆alkyl, C₁₋₆fluoroalkyl, —C₁₋₆alkylene-OH, —C₁₋₆alkylene-OC₁₋₄alkyl, G⁵, or —C₁₋₃alkylene-G⁵;
wherein alternatively, R^{5a} and R^{5b}, together with the nitrogen to which they attach form a 4- to 10-membered heterocyclic ring containing the nitrogen attached to R^{5a} and R^{5b} and optionally 1-2 additional heteroatoms that are independently O, N, or S, the heterocyclic ring being optionally substituted with 1-4 substituents independently selected from the group consisting of halogen, cyano, C₁₋₄alkyl, C₁₋₄fluoroalkyl, —C₁₋₆alkylene-OH, oxo, —OR⁵⁰, —N(R⁵⁰)₂, —NR⁵⁰C(O)R⁵⁰, —NR⁵⁰SO₂R⁵⁰, —C(O)OR⁵O, —C(O)N(R⁵⁰)₂, —SO₂R⁵⁰, G^{5a}, and —C₁₋₃ alkylene-G^{5a};
G⁵ is a phenyl, a 5- to 6-membered heteroaryl containing 1-3 heteroatoms, a 4- to 8-membered heterocyclyl containing 1-2 heteroatoms, or a C₃₋₆cycloalkyl, wherein the heteroatoms are independently selected from the group consisting of O, N, and S, and G⁵ is optionally substituted with 1-5 substituents independently selected from the group consisting of halogen, cyano, C₁₋₄alkyl, C₁₋₄fluoroalkyl, —C₁₋₆alkylene-OH, oxo, —OR⁵⁰, —N(R⁵⁰)₂, —NR⁵⁰C(O)R⁵⁰, —NR⁵⁰SO₂R⁵⁰, —C(O)OR⁵⁰, —C(O)N(R⁵⁰)₂, —SO₂R⁵⁰, G^{5a}, and —C₁₋₃alkylene-G^{5a}.
R⁵⁰, at each occurrence, is independently hydrogen, C₁₋₄alkyl, C₁₋₄fluoroalkyl, C₃₋₆cycloalkyl, or —C₁₋₃alkylene-C₃₋₆cycloalkyl, wherein each cycloalkyl is optionally substituted with 1-4 substituents independently selected from the group consisting of halogen, C₁₋₄alkyl, and C₁₋₄ fluoroalkyl;
G^{5a}, at each occurrence, is independently a phenyl, a 5- to 6-membered heteroaryl containing 1-3 heteroatoms, a 4- to 8-membered heterocyclyl containing 1-2 heteroatoms, or a 3- to 8-membered carbocyclyl, wherein the heteroatoms are independently selected from the group consisting of O, N, and S, and G^{5a} is optionally substituted with 1-5 substituents independently selected from the group consisting of halogen, cyano, C₁₋₄alkyl, C₁₋₄fluoroalkyl, oxo, OH, —OC₁₋₄alkyl, —OC₁₋₄fluoroalkyl, C₃₋₄cycloalkyl, and —C₁₋₃alkylene-C₃₋₄ cycloalkyl;
R⁶ is hydrogen, halogen, C₁₋₄alkyl, C₁₋₄fluoroalkyl, —OR^{6a}, —N(R^{6a})₂, —C₁₋₃alkylene-OR^{6a}, or C₃₋₆cycloalkyl;
R^{6a}, at each occurrence, is independently hydrogen, C₁₋₄alkyl, C₁₋₄fluoroalkyl, C₃₋₄cycloalkyl, or —C₁₋₃alkylene-C₃₋₄cycloalkyl;
wherein alternatively, two R^{6a}, together with the nitrogen to which they attach form a 4- to 8-membered heterocyclic ring containing the nitrogen attached to R^{6a} and optionally 1-2 additional heteroatoms that are independently O, N, or S, the heterocyclic ring being optionally substituted with 1-4 substituents independently selected from the group consisting of halogen, C₁₋₂alkyl, and C₁₋₂fluoroalkyl;
R⁷ is hydrogen, C₁₋₄alkyl, C₁₋₄fluoroalkyl, —OR^{7a}, —C₁₋₃alkylene-OR^{7a}, or C₃₋₆cycloalkyl;
R^{7a} is hydrogen, C₁₋₄alkyl, C₁₋₄fluoroalkyl, C₃₋₄cycloalkyl, or —C₁₋₃alkylene-C₃₋₄cycloalkyl;
R⁸, at each occurrence, is independently halogen, C₁₋₄alkyl, C₁₋₄fluoroalkyl, or C₃₋₄cycloalkyl; and n is 0, 1, 2, 3, or 4.

E1.1. The compound of E1, or a pharmaceutically acceptable salt thereof, wherein R² is halogen, cyano, NO₂, C₁₋₆alkyl, C₁₋₆haloalkyl, —OR^{b}, —NR^{b}R^{c}, —NR^{c}C(O)R^{b}, —NR^{c}SO₂R^{a}, —N═S(O)(R^{a})₂, —P(O)(R^{a})₂, G², or —C₁₋₃alkylene-G².

E1.2. The compound of E1, or a pharmaceutically acceptable salt thereof, R² is hydrogen.

E2. The compound of any of E1-E1.2, or a pharmaceutically acceptable salt thereof, wherein R¹ is hydrogen.

E2.1. The compound of any of E1-E1.2, or a pharmaceutically acceptable salt thereof, wherein R¹ is C₁₋₄alkyl.

E2.2. The compound of E2.1, or a pharmaceutically acceptable salt thereof, wherein R¹ is methyl.

E3. The compound of any of E1-E2.2, or a pharmaceutically acceptable salt thereof, wherein R³ is hydrogen.

E4. The compound of any of E1, E1.1, or E2-E3, or a pharmaceutically acceptable salt thereof, R² is C₁₋₆haloalkyl, —OR^{b}, —NR^{b}R^{c}, —NR^{b}C(O)R^{c}, —NR^{b}SO₂R^{a}, —N═S(O)(R^{a})₂, —P(O)(R^{a})₂, G², or —C₁₋₃alkylene-G².

E5. The compound of E4, or a pharmaceutically acceptable salt thereof, wherein R² is C₁₋₆haloalkyl.

E5.1. The compound of E5, or a pharmaceutically acceptable salt thereof, wherein R² is C₁₋₆fluoroalkyl.

E5.2. The compound of E5.1, or a pharmaceutically acceptable salt thereof, wherein R² is CF₃.

E6. The compound of E4, or a pharmaceutically acceptable salt thereof, wherein R² is —OR^{b}; and R^{b} is G² or —C₁₋₃alkylene-G².

E6.1. The compound of E6, or a pharmaceutically acceptable salt thereof, wherein R^{b} is G².

E6.2. The compound of E6, or a pharmaceutically acceptable salt thereof, wherein R^{b} is —C₁₋₃alkylene-G².

E6.3. The compound of any of E6-E6.2, or a pharmaceutically acceptable salt thereof, wherein G² is the optionally substituted 6- to 12-membered aryl.

E6.4. The compound of any of E6-E6.3, or a pharmaceutically acceptable salt thereof, wherein the ring system of the optionally substituted 6- to 12-membered aryl at G² is phenyl.

E6.5. The compound of E6.4, or a pharmaceutically acceptable salt thereof, wherein G² is phenyl optionally substitued with 1-3 halogen (e.g., optionally substituted with 1-3 fluoro such as 2-fluorophenyl).

E6.6. The compound of any of E6-E6.2, or a pharmaceutically acceptable salt thereof, wherein G² is the optionally substituted 4- to 12-membered heterocyclyl.

E6.7. The compound of any of E6-E6.2 or E6.6, or a pharmaceutically acceptable salt thereof, wherein the ring system of the optionally substituted 4- to 12-membered heterocyclyl at G² is 4- to 8-membered heterocyclyl ring system containing 1-2 heteroatoms independently selected from the group consisting of O, N, and S.

E6.8. The compound of E6.7, or a pharmaceutically acceptable salt thereof, wherein the ring system of the 4- to 8-membered heterocyclyl at G² is oxazolidin-3-yl.

E6.9. The compound of E6.8, or a pharmaceutically acceptable salt thereof, wherein G² is 2-oxooxazolidin-3-yl.

E7. The compound of E4, or a pharmaceutically acceptable salt thereof, wherein R² is —NR^{b}R^{c}; R^{b} is C₁₋₆alkyl, G², or —C₁₋₃alkylene-G²; and R^{c} is hydrogen or C₁₋₆alkyl.

E7.1. The compound of E7, or a pharmaceutically acceptable salt thereof, wherein R^{b} is C₁₋₆alkyl.

E7.1a. The compound of E7.1, or a pharmaceutically acceptable salt thereof, wherein R^{b} is CH₂CH(CH₃)₂.

E7.2. The compound of E7, or a pharmaceutically acceptable salt thereof, wherein R^{b} is G².

E7.3. The compound of E7, or a pharmaceutically acceptable salt thereof, wherein R^{b} is —C₁₋₃alkylene-G².

E7.4. The compound of E7.3, or a pharmaceutically acceptable salt thereof, wherein R^{b} is —CH₂-G².

E7.5. The compound of any of E7 or E7.2-E7.4, or a pharmaceutically acceptable salt thereof, wherein G² is the optionally substituted 6- to 12-membered aryl.

E7.6. The compound of any of E7 or E7.2-E7.5, or a pharmaceutically acceptable salt thereof, wherein the ring system of the optionally substituted 6- to 12-membered aryl at G² is phenyl.

E7.7. The compound of any of E7 or E7.2-E7.4, or a pharmaceutically acceptable salt thereof, wherein G² is the optionally substituted 5- to 12-membered heteroaryl.

E7.8. The compound of any of E7, E7.2-E7.4 or E7.7, or a pharmaceutically acceptable salt thereof, wherein the ring system of the optionally substituted 5- to 12-membered heteroaryl at G² is a 5- to 6-membered heteroaryl containing 1-3 heteroatoms independently selected from the group consisting of O, N, and S.

E7.9. The compound of E7.8, or a pharmaceutically acceptable salt thereof, wherein the ring system of the optionally substituted 5- to 6-membered heteroaryl at G² is pyrazolyl, imidazolyl, oxazolyl, thiazolyl, pyridinyl, pyrazinyl, pyridazinyl, or pyrimidinyl.

E7.10. The compound of any of E7 or E7.2-E7.9, or a pharmaceutically acceptable salt thereof, wherein G² is optionally substituted with a first substituent selected from the group consisting of halogen (e.g., fluoro, chloro), cyano, C₁₋₆alkyl (e.g., methyl, isopropyl), C₁₋₆ haloalkyl (e.g., C₁₋₄fluoroalkyl such as CF₃), —OR^{x} (e.g., —OCH₃, —OCHF₂, —OCF₃), —N(R^{x})₂, —C(O)R^{x}, —C(O)OR^{x}, —C(O)N(R^{x})₂, —C₁₋₆alkylene-OR^{x}, —C₁₋₆alkylene-N(R^{x})₂, G^{2a} (e.g., thiazolyl, cyclopropyl) and —C₁₋₃alkylene-G^{2a}; and further optionally substituted with 1-3 substituents independently selected from the group consisting of halogen, C₁₋₄alkyl, C₁₋₄ fluoroalkyl, and —OC₁₋₄alkyl.

E7.10a. The compound of E7.10, or a pharmaceutically acceptable salt thereof, wherein G² is optionally substituted with a first substituent selected from the group consisting of fluoro, chloro, cyano, methyl, isopropyl, CF₃, —OCH₃, —OCHF₂, —OCF₃, 2-methylthiazol-4-yl, and cyclopropyl; and further optionally substituted with 1-3 substituents independently selected from the group consisting of fluoro, chloro, methyl, CF₃, and —OCH₃.

E7.11. The compound of any of E7 or E7.2-E7.4, or a pharmaceutically acceptable salt thereof, wherein G² is the optionally substituted 4- to 12-membered heterocyclyl.

E7.12. The compound of any of E7, E7.2-E7.4, or E7.11, or a pharmaceutically acceptable salt thereof, wherein the ring system of the optionally substituted 4- to 12-membered heterocyclyl at G² is a 4- to 7-membered heterocyclyl containing 1-2 heteroatoms that are oxygen.

E7.13. The compound of any of E7 or E7.2-E7.4, or a pharmaceutically acceptable salt thereof, wherein G² is the optionally substituted 3- to 12-membered carbocyclyl.

E7.14. The compound of any of E7, E7.2-E7.4, or E7.13, or a pharmaceutically acceptable salt thereof, wherein the ring system of the optionally substituted C₃₋₁₂carbocyclyl at G² is a C₃₋₆cycloalkyl.

E7.15. The compound of any of E7.11-E7.14, wherein G² is optionally substituted with 1-4 C₁₋₄alkyl (e.g., methyl).

E7.16. The compound of E7.5, or a pharmaceutically acceptable salt thereof, wherein G² is a phenyl fused to a 5- to 7-membered heterocycle containing 1-2 oxygen atoms

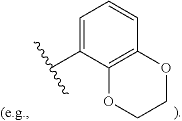 [SMILES: *c1cccc2c1OCCO2]

E7.17a. The compound of E7.2, or a pharmaceutically acceptable salt thereof,
wherein R² is

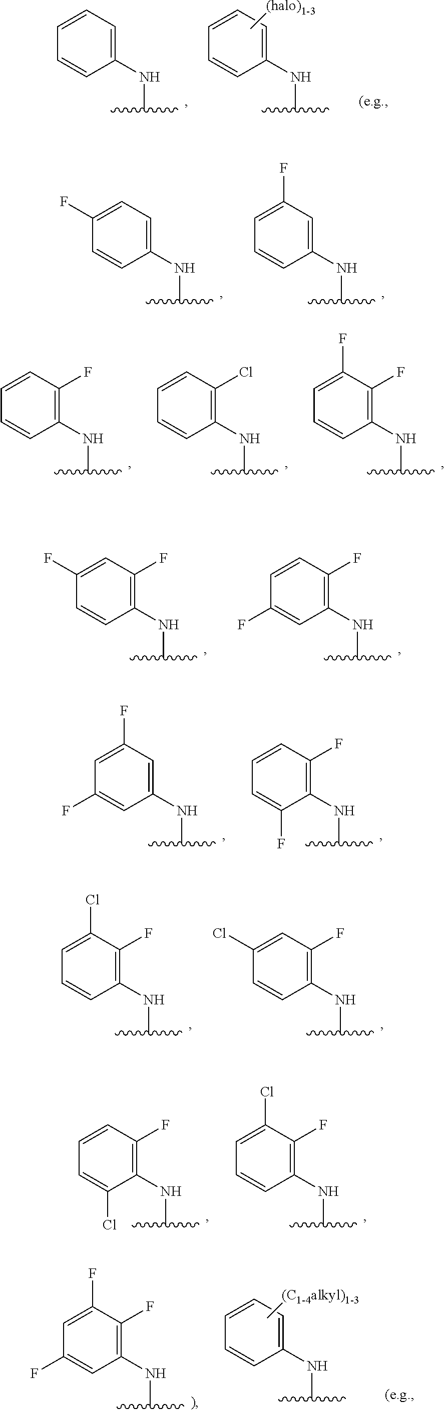

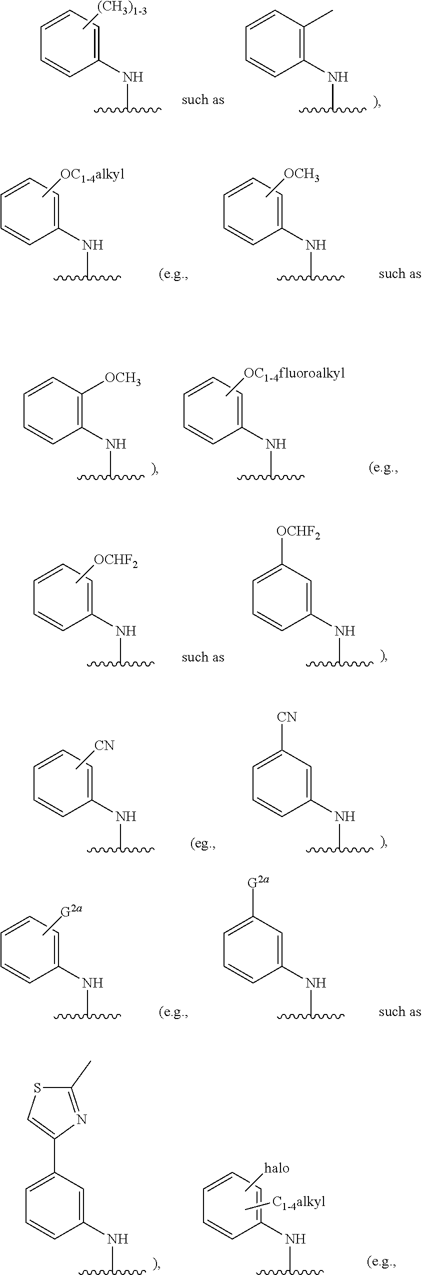

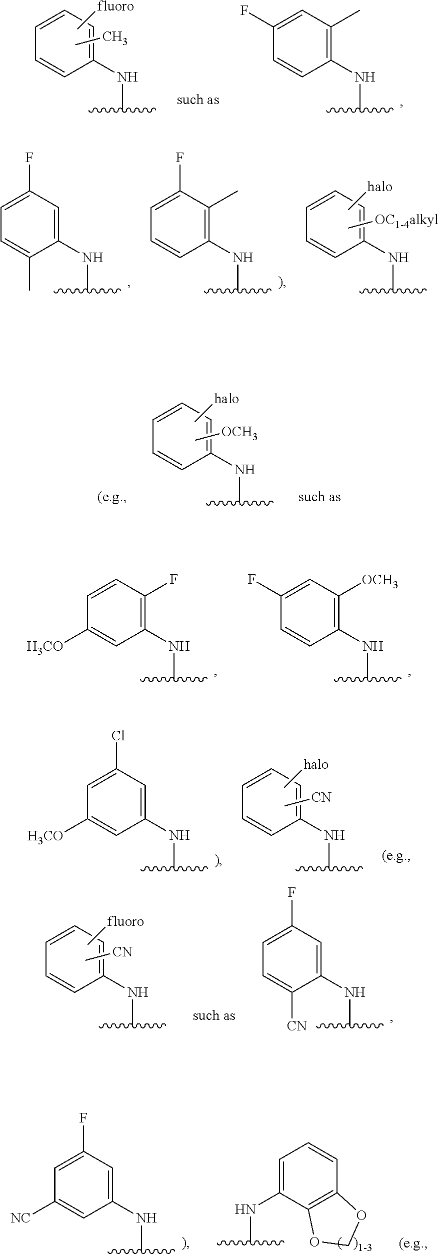

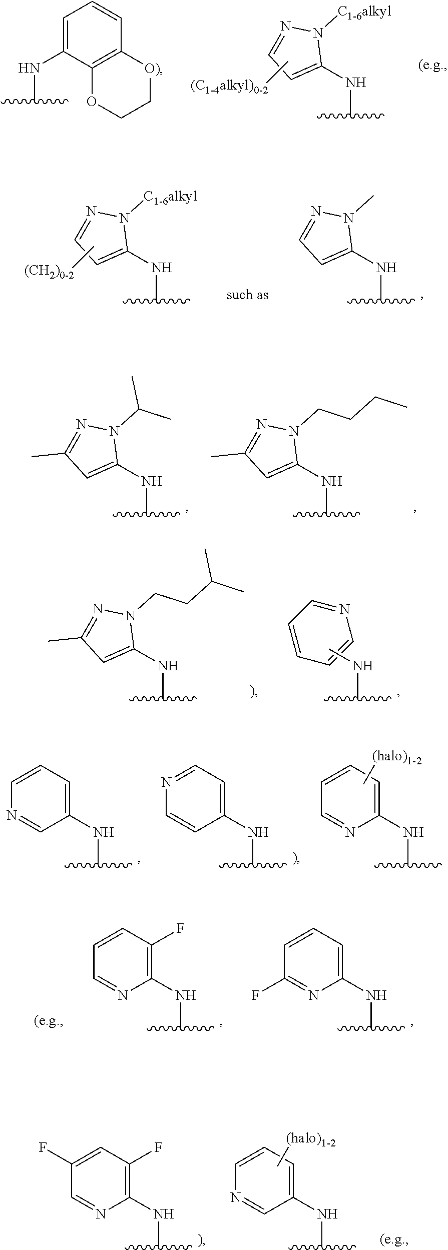

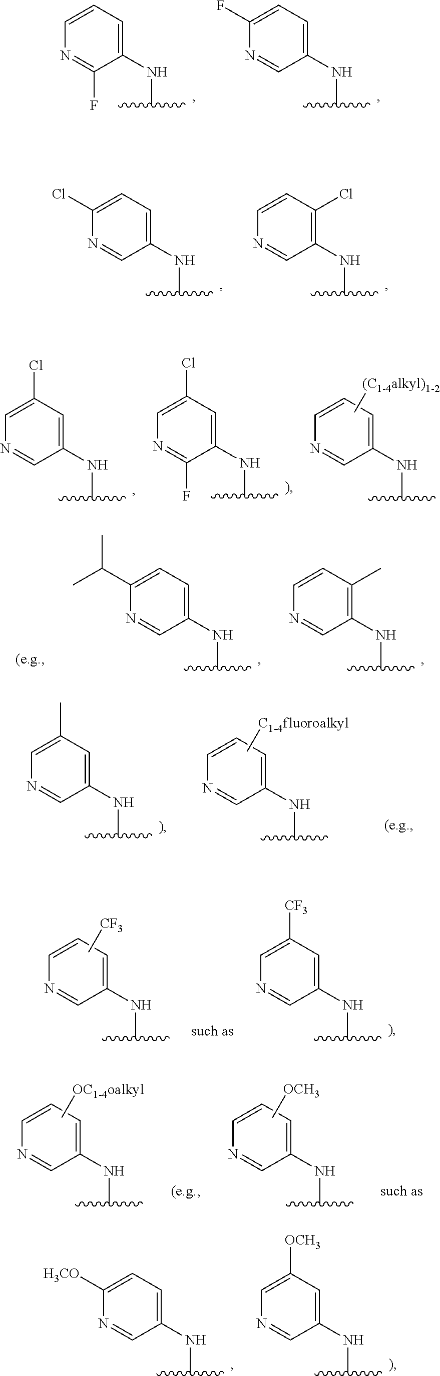

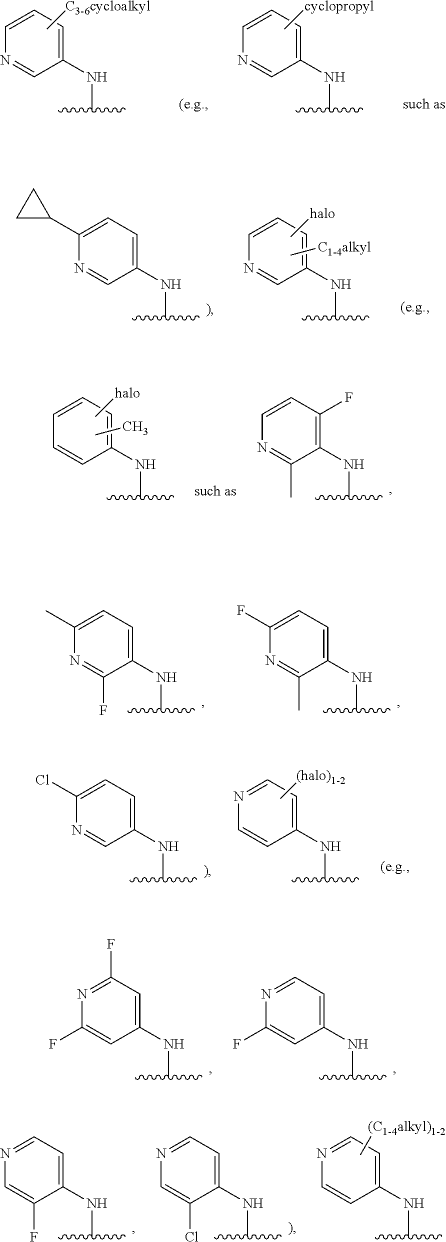

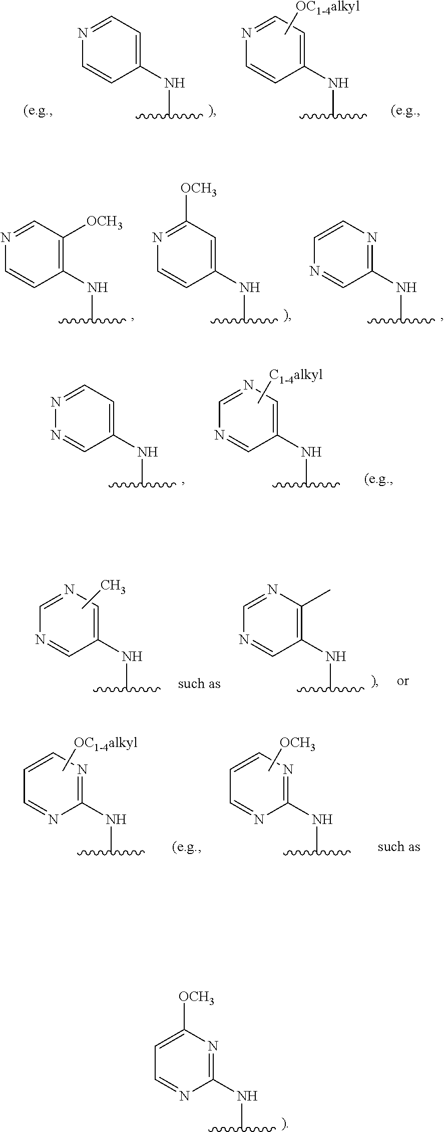

E7.17b. The compound of E7.2, or a pharmaceutically acceptable salt thereof, wherein R²

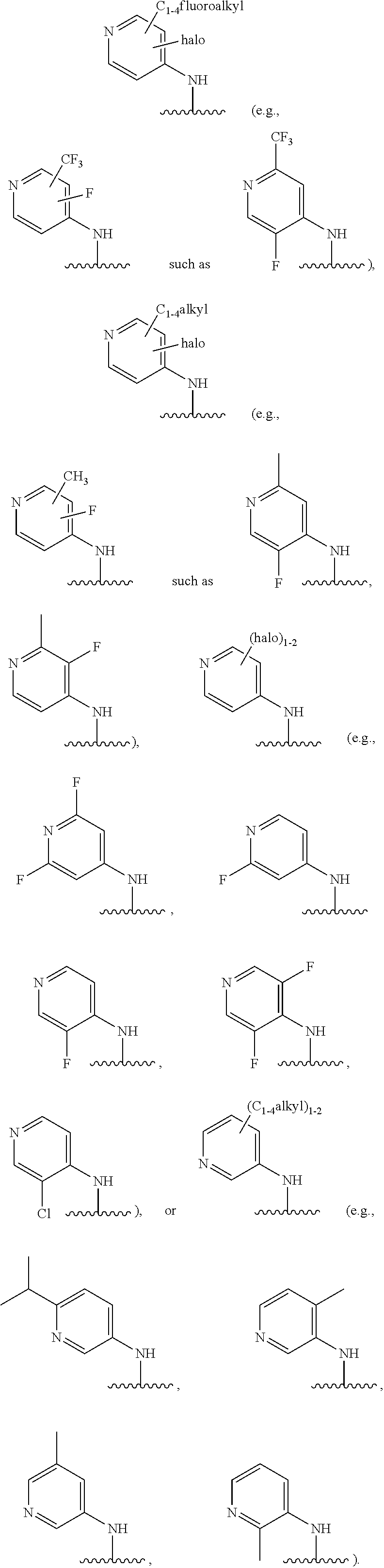

E7.17c. The compound of E7.17b, or a pharmaceutically acceptable salt thereof, wherein R² is

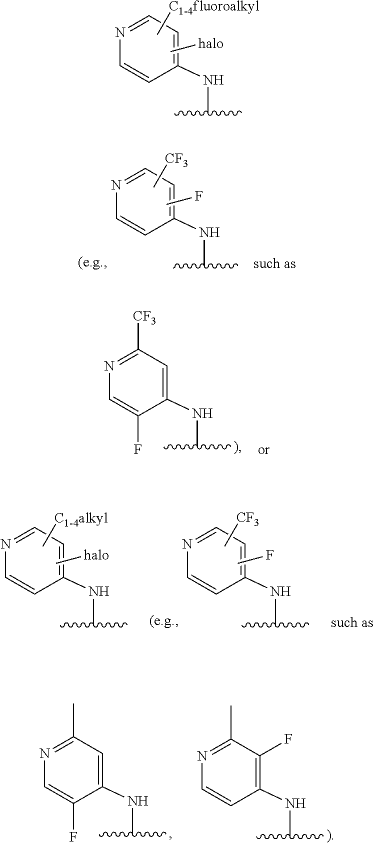

E7.18. The compound of E7.4, or a pharmaceutically acceptable salt thereof, wherein R² is

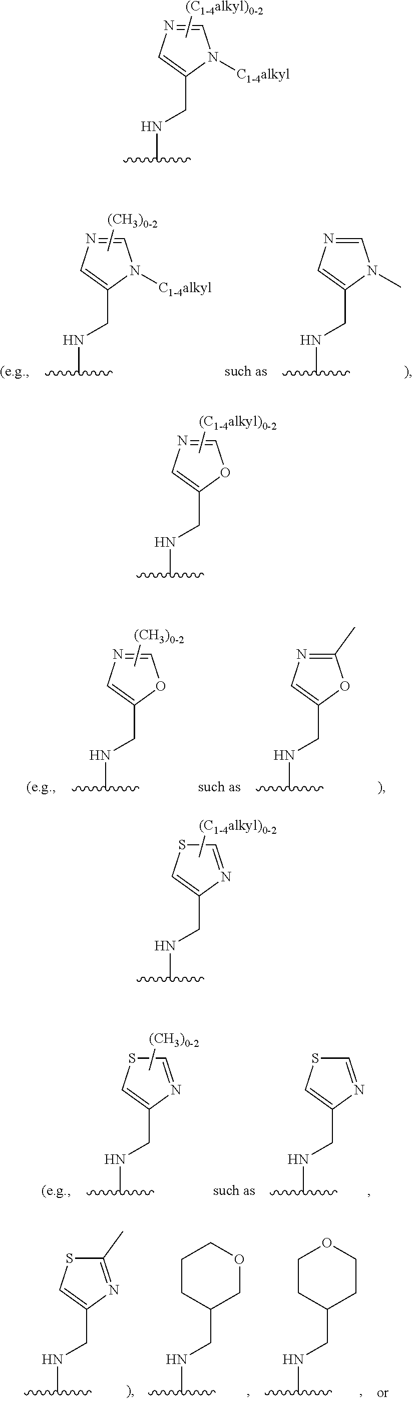

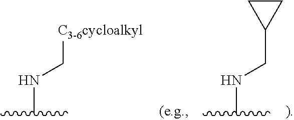

E7.19. The compound of E7.17a or E7.17b, or a pharmaceutically acceptable salt thereof, wherein R² is

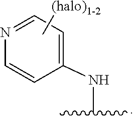

E7.20. The compound of E7.19, or a pharmaceutically acceptable salt thereof, wherein R² is

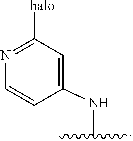

E7.21. The compound of E7.20, or a pharmaceutically acceptable salt thereof, wherein R² is

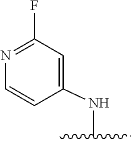 [SMILES: *Nc1ccnc(F)c1]

E7.22. The compound of E7.17a, or a pharmaceutically acceptable salt thereof, wherein R² is

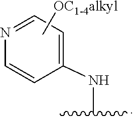

E7.23. The compound of E7.22, or a pharmaceutically acceptable salt thereof, wherein R² is

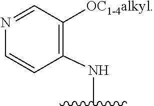

E7.24. The compound of E7.23, or a pharmaceutically acceptable salt thereof, wherein R² is

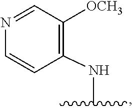 [SMILES: *Nc1ccncc1OC]

E7.25. The compound of any of E7-E7.16, or a pharmaceutically acceptable salt thereof, wherein R is hydrogen.

E7.26. The compound of any of E7-E7.16 or E7.25, or a pharmaceutically acceptable salt thereof, wherein R^{x} is hydrogen, C₁₋₄alkyl, C₁₋₄fluoroalkyl, C₃₋₄cycloalkyl, or —CH₂—C₃₋₄cycloalkyl.

E7.27. The compound of E7.26, or a pharmaceutically acceptable salt thereof, wherein R^{x} is C₁₋₄alkyl or C₁₋₂fluoroalkyl.

E8. The compound of E4, or a pharmaceutically acceptable salt thereof, wherein R² is —NR^{c}C(O)R^{b};
R^{b} is G²; and
R^{c} is hydrogen.

E8.1. The compound of E8, or a pharmaceutically acceptable salt thereof, wherein G² is the optionally substituted C₃₋₁₂carbocyclyl.

E8.2. The compound of E8 or E8.1, or a pharmaceutically acceptable salt thereof, wherein the ring system of the optionally substituted C₃₋₁₂carbocyclyl at G² is a C₃₋₆cycloalkyl.

E8.3. The compound of E8.2, or a pharmaceutically acceptable salt thereof, wherein G² is C₃₋₆cycloalkyl.

E8.4. The compound of E8.3, or a pharmaceutically acceptable salt thereof, wherein G² is cyclopropyl.

E8.5. The compound of E8, or a pharmaceutically acceptable salt thereof, wherein G² is the optionally substituted 4- to 12-membered heterocyclyl.

E8.6. The compound of E8 or E8.5, or a pharmaceutically acceptable salt thereof, wherein the ring system of the optionally substituted 4- to 12-membered heterocyclyl at G² is a 4- to 7-membered heterocyclyl containing 1-2 heteroatoms that are oxygen.

E8.7. The compound of E8.6, or a pharmaceutically acceptable salt thereof, wherein G² is oxetanyl or tetrahydropyranyl (e.g., oxetan-3-yl, tetrahydropyran-4-yl).

E9. The compound of E4, or a pharmaceutically acceptable salt thereof, wherein R² is —NR^{c}SO₂R^{a}.

R^{a} is C₁₋₆alkyl or G²; and R^{c} is hydrogen.

E9.1. The compound of E9, or a pharmaceutically acceptable salt thereof wherein R^{a} is C₁₋₆alkyl.

E9.2. The compound of E9.1, or a pharmaceutically acceptable salt thereof wherein R^{a} is methyl.

E9.3. The compound of E9, or a pharmaceutically acceptable salt thereof wherein R^{a} is G².

E9.4. The compound of E9.3, or a pharmaceutically acceptable salt thereof wherein G² is the optionally substituted C₃₋₁₂carbocyclyl.

E9.5. The compound of any of E9, E9.3 or E9.4, or a pharmaceutically acceptable salt thereof, wherein the ring system of the C₃₋₁₂carbocyclyl at G² is a C₃₋₆cycloalkyl.

E9.6. The compound of E9.5, or a pharmaceutically acceptable salt thereof, wherein G² is C₃₋₆cycloalkyl optionally substituted with 1-4 substituents independently selected from the group consisting of fluoro, chloro, and C₁₋₄alkyl (e.g., methyl).

E9.7. The compound of E9.6, or a pharmaceutically acceptable salt thereof, wherein G² is cyclopropyl, 3,3-difluorocyclobutyl, or 1-methylcyclobutyl.

E9.8. The compound of E9.3, or a pharmaceutically acceptable salt thereof wherein G² is the optionally substituted 5- to 12-membered heteroaryl.

E9.9. The compound of any of E9, E9.3, or E9.8, or a pharmaceutically acceptable salt thereof, wherein the ring system of the 5- to 12-membered heteroaryl at G² is a 5- to 6-membered heteroaryl containing 1-3 heteroatoms independently selected from the group consisting of O, N, and S.

E9.10. The compound of E9.9, or a pharmaceutically acceptable salt thereof, wherein the ring system of the 5- to 6-membered heteroaryl at G² is oxazolyl or thiazolyl (e.g., oxazol-2-yl, thiazol-4-yl).

E9.11. The compound of any of E9.8-E9.10, or a pharmaceutically acceptable salt thereof, wherein G² is optionally substituted with 1-3 C₁₋₄alkyl (e.g., methyl).

E9.12. The compound of E9.11, or a pharmaceutically acceptable salt thereof, wherein G² is oxazol-2-yl or 2-methylthiazol-4-yl.

E10. The compound of E4, or a pharmaceutically acceptable salt thereof, wherein R² is —N═S(O)(R^{a})₂; and
the two R^{a} of —N═S(O)(R^{a})₂ join together as a straight alkylene chain to form a 5- to 7-membered heterocyclic ring.

E10.1. The compound of E10, or a pharmaceutically acceptable salt thereof, wherein R² is

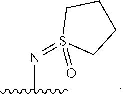 [SMILES: *N=S1(=O)CCCC1]

E11. The compound of E4, or a pharmaceutically acceptable salt thereof, wherein R² is —P(O)(R^{a})₂; and R^{a}, at each occurrence is independently C₁₋₆alkyl.

E11.1. The compound of E11, or a pharmaceutically acceptable salt thereof, wherein R^{a} is methyl.

E12. The compound of E4, or a pharmaceutically acceptable salt thereof, wherein R² is G².

E12.1. The compound of E12, or a pharmaceutically acceptable salt thereof, wherein G² is the optionally substituted 6- to 12-membered aryl.

E12.2. The compound of E12 or E12.1, or a pharmaceutically acceptable salt thereof, wherein the ring system of the optionally substituted 6- to 12-membered aryl at G² is phenyl.

E12.3a. The compound of E12.2, or a pharmaceutically acceptable salt thereof, wherein G² is

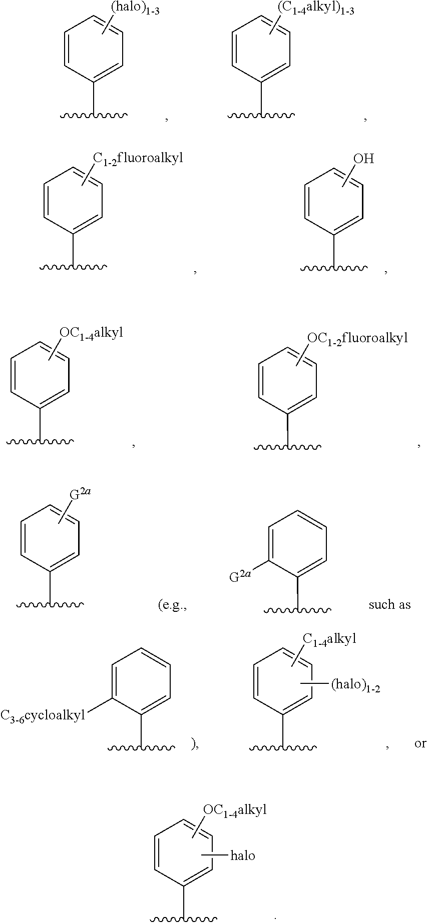

E12.3b. The compound of E12.2, or a pharmaceutically acceptable salt thereof, wherein G² is

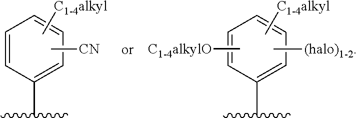

E12.4a. The compound of E12.3a, or a pharmaceutically acceptable salt thereof, wherein G² is

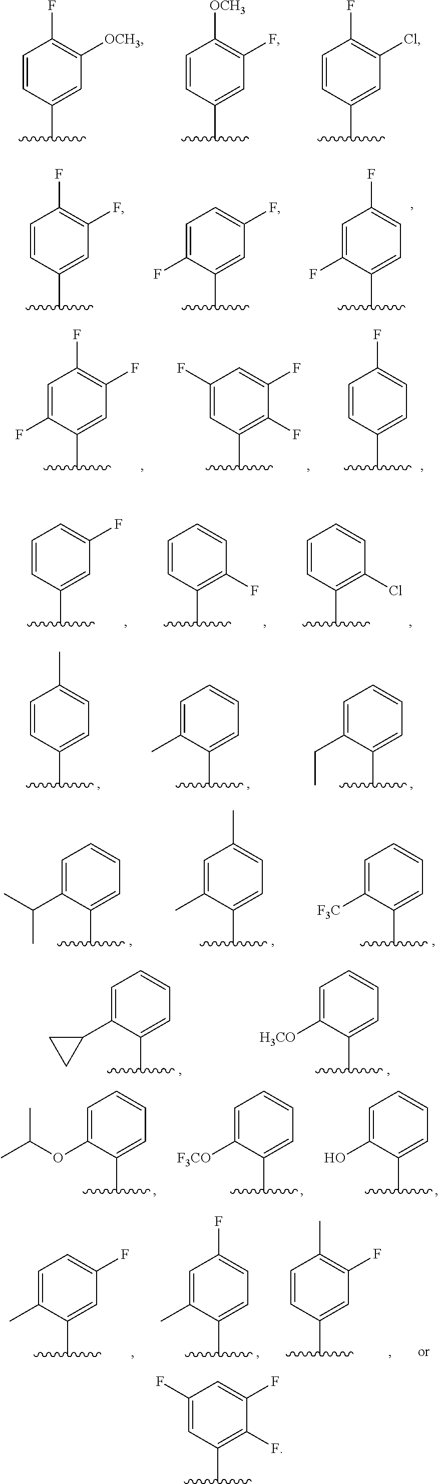

E12.4b. The compound of E12.3a, or a pharmaceutically acceptable salt thereof, wherein G² is

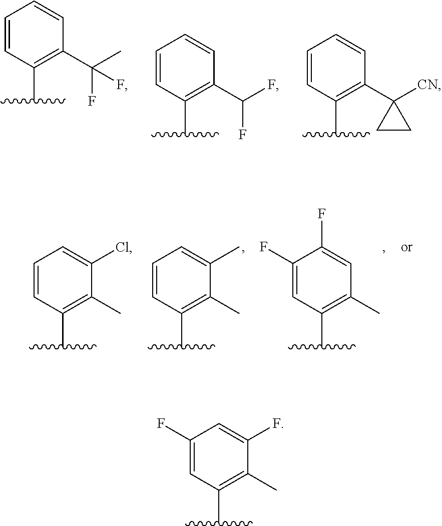

E12.4c. The compound of E12.3b, or a pharmaceutically acceptable salt thereof, wherein G² is

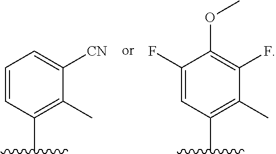

E12.5. The compound of E12, or a pharmaceutically acceptable salt thereof, wherein G² is the optionally substituted 5- to 12-membered heteroaryl.

E12.6. The compound of E12 or E12.5, or a pharmaceutically acceptable salt thereof, wherein the ring system of the optionally substituted 5- to 12-membered heteroaryl at G² is a 5- to 6-membered heteroaryl containing 1-3 heteroatoms independently selected from the group consisting of O, N, and S.

E12.7. The compound of E12.6, or a pharmaceutically acceptable salt thereof, wherein the ring system of the optionally substituted 5- to 6-membered heteraryl at G² is pyrrolyl, pyrazolyl, imidazolyl, 1,2,3-triazolyl, furanyl, thiophenyl, oxazolyl, isoxazolyl, thiazolyl, pyridinyl, pyrimidinyl, pyrazinyl, or pyridazinyl.

E12.8. The compound of E12.7, or a pharmaceutically acceptable salt thereof, wherein the ring system of the optionally substituted 5- to 6-membered heteraryl at G² is pyrrol-4-yl, furan-2-yl, thiophen-2-yl, thiophen-3-yl, pyrazol-1-yl, pyrazol-3-yl, pyrazol-4-yl, pyrazol-5-yl, imidazol-2-yl, imidazol-5-yl, oxazol-5-yl, isoxazol-4-yl, thiazol-4-yl, thiazol-5-yl, 1,2,3-triazol-4-yl, pyridin-2-yl, pyridin-3-yl, pyridin-4-yl, pyrimidin-5-yl, pyrazin-2-yl, pyridazin-3-yl, or pyridazin-4-yl.

E12.9. The compound of E12.7, or a pharmaceutically acceptable salt thereof, wherein G² is

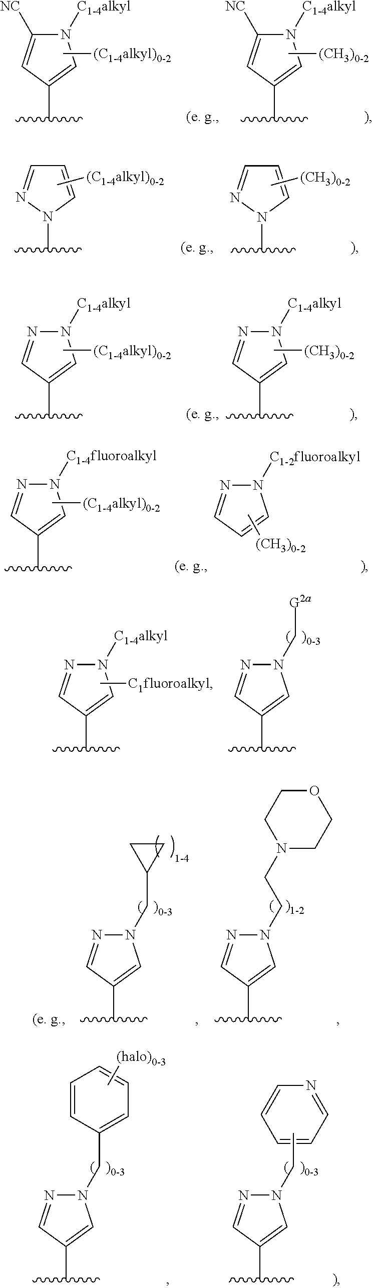

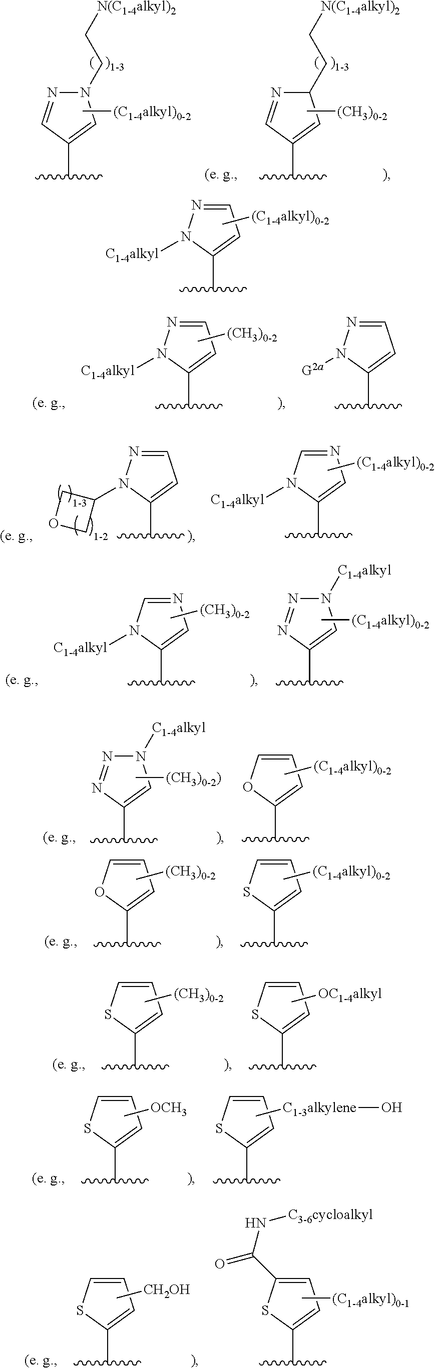

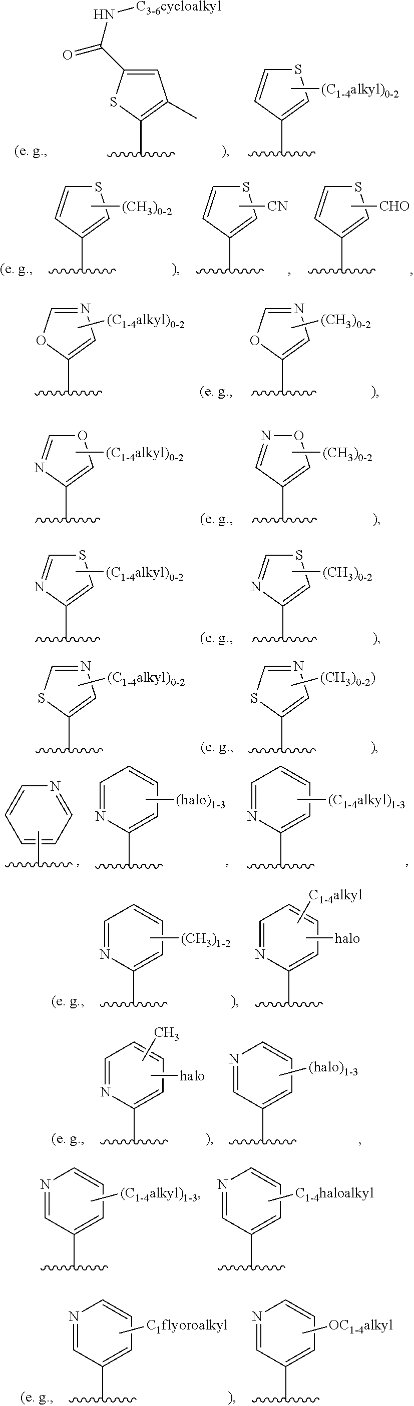

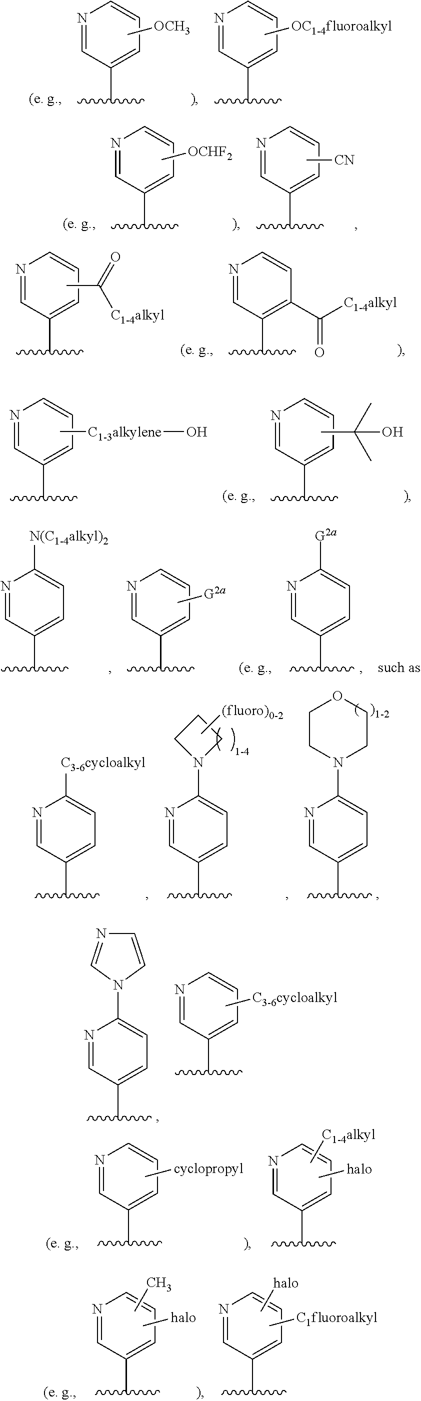

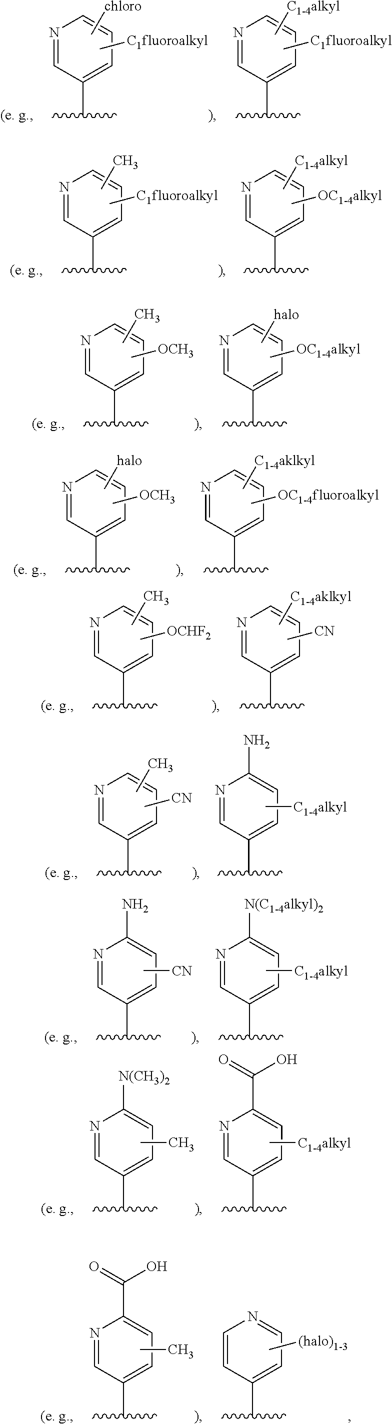

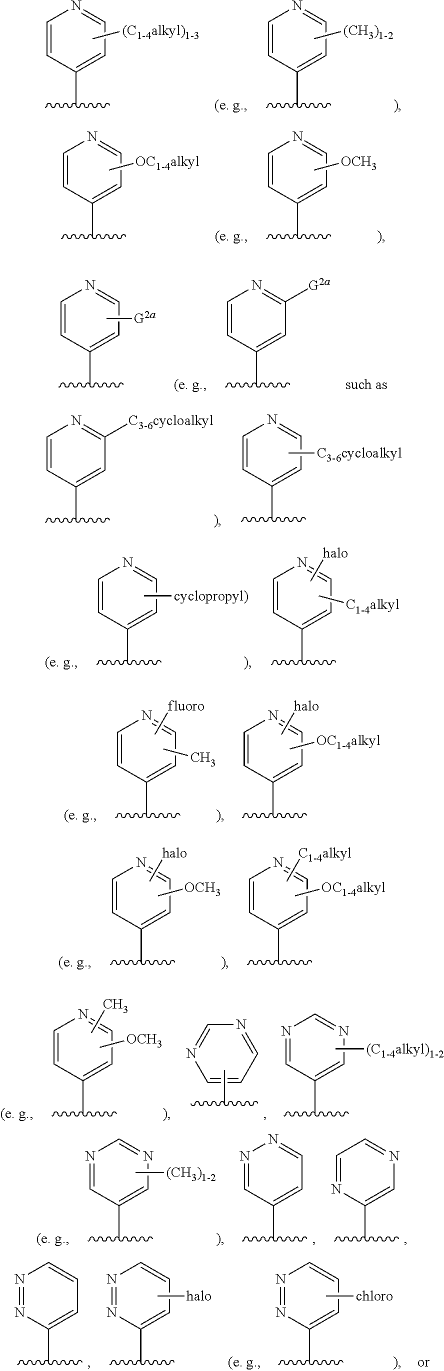

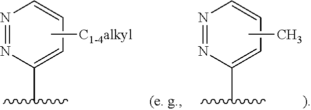

E12.10. The compound of E12.9, or a pharmaceutically acceptable salt thereof, wherein G² is

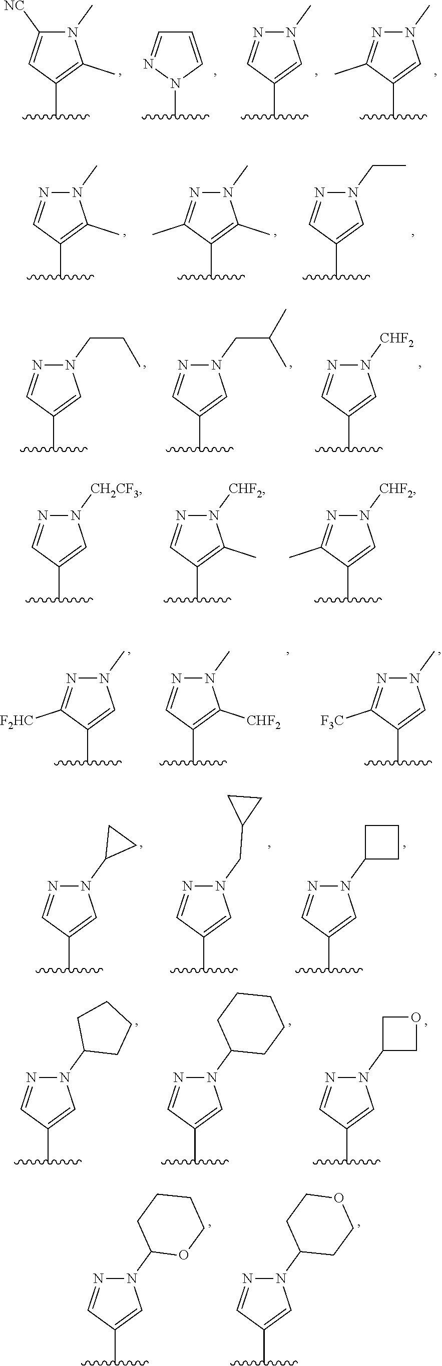

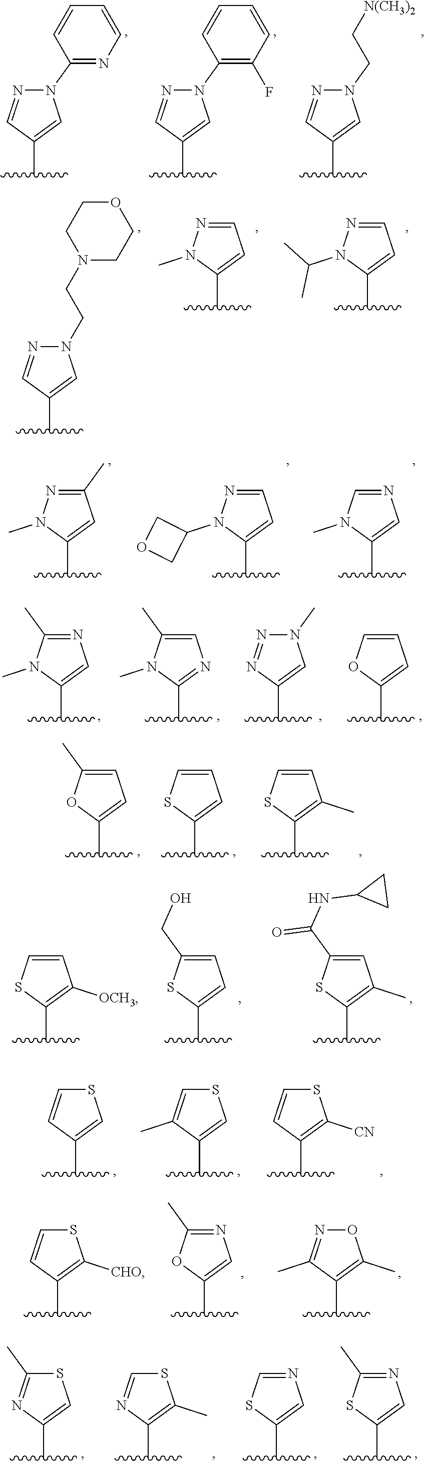

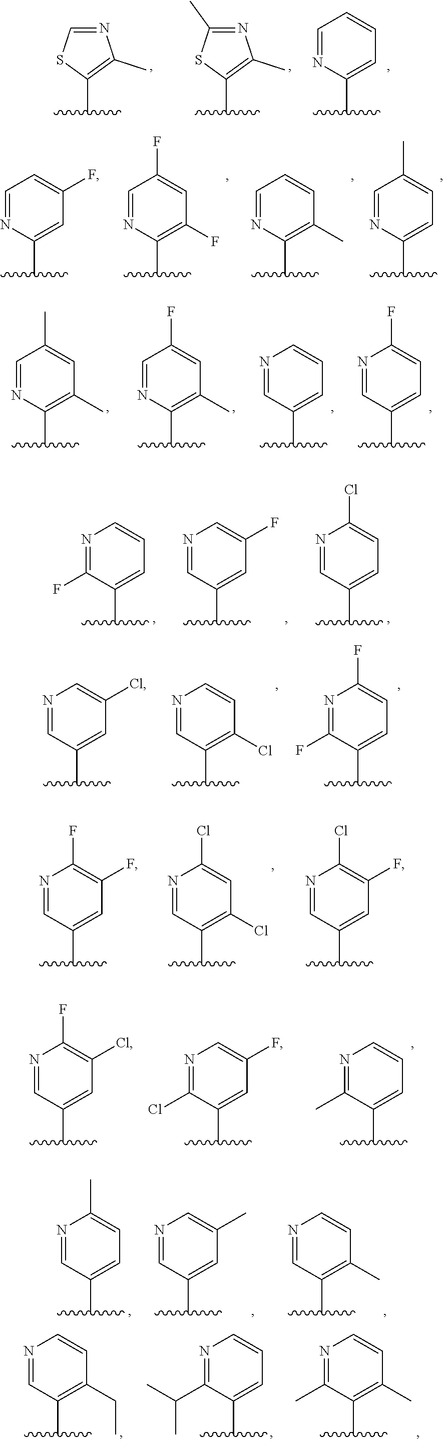

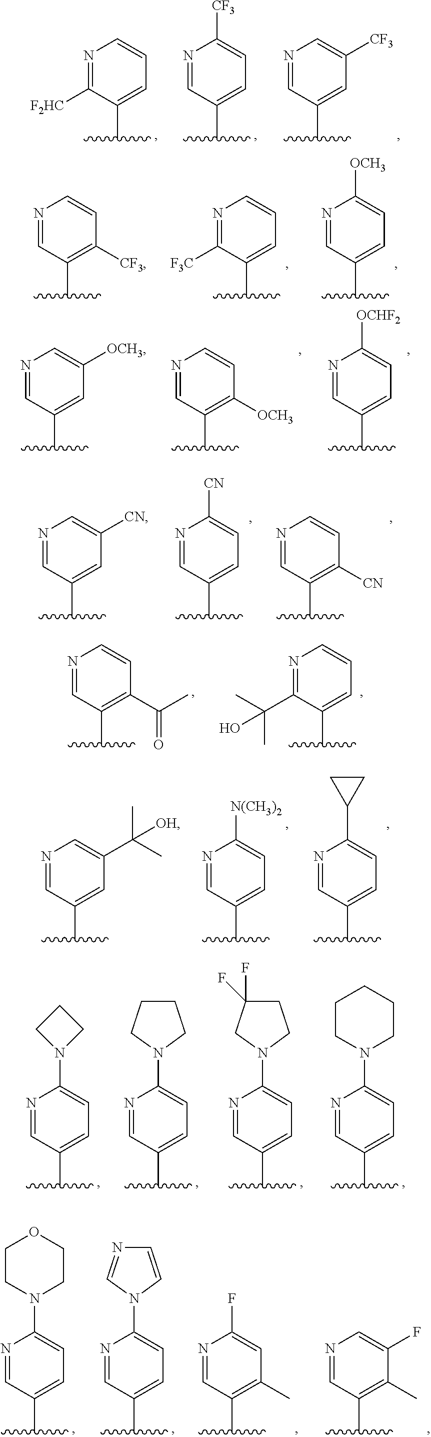

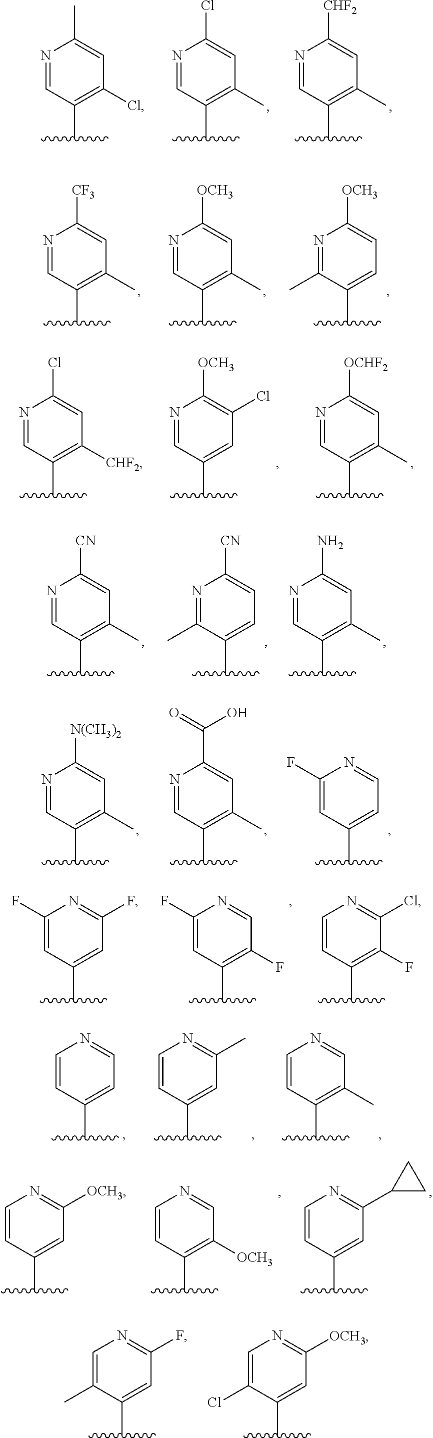

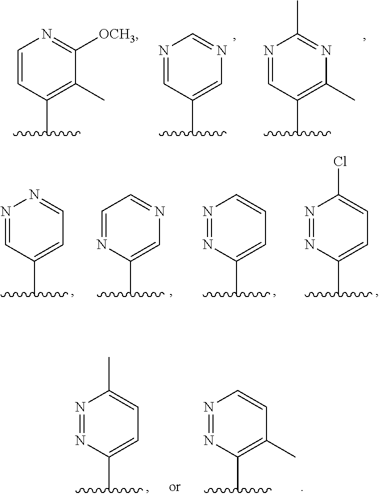

E12.11. The compound of E12 or E12.5, or a pharmaceutically acceptable salt thereof, wherein the ring system of the optionally substituted 5- to 12-membered heteroaryl at G² is a 9- to 10-membered heteroaryl containing 1-3 heteroatoms independently selected from the group consisting of O, N, and S.

E12.12a. The compound of E12.11, or a pharmaceutically acceptable salt thereof, wherein the ring system of the 9- to 10-membered heteroaryl at G² is quinolinyl, imidazo[1,2-a]pyridinyl, pyrazolo[1,5-a]pyridinyl, or 6,7-dihydro-4H-pyrazolo[5,1-c][1,4]oxazin-3-yl.

E12.12b. The compound of E12.11, or a pharmaceutically acceptable salt thereof, wherein the ring system of the 9- to 10-membered heteroaryl at G² is indolyl.

E12.13a. The compound of E12.12a, or a pharmaceutically acceptable salt thereof, wherein the ring system of the 9- to 10-membered heteroaryl at G² is quinolin-3-yl, quinolin-5-yl, imidazo[1,2-a]pyridin-6-yl, pyrazolo[1,5-a]pyridin-3-yl, or 6,7-dihydro-4H-pyrazolo[5,1-c][1,4]oxazin-3-yl.

E12.13b. The compound of E12.12b, or a pharmaceutically acceptable salt thereof, wherein the ring system of the 9- to 10-membered heteroaryl at G² is indol-4-yl.

E12.14a. The compound of E12.12a, or a pharmaceutically acceptable salt thereof, wherein G² is

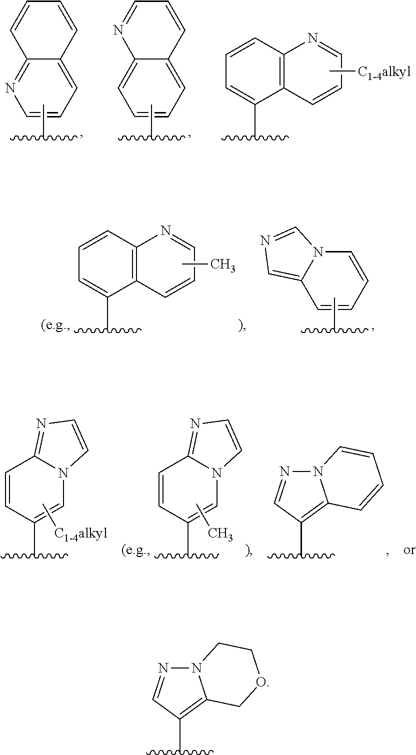

E12.14b. The compound of E12.12b or 12.13b, or a pharmaceutically acceptable salt thereof, wherein G² is

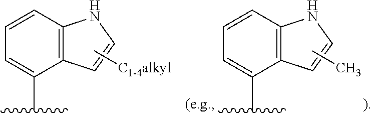

E12.15a. The compound of E12.14a, or a pharmaceutically acceptable salt thereof, wherein G² is

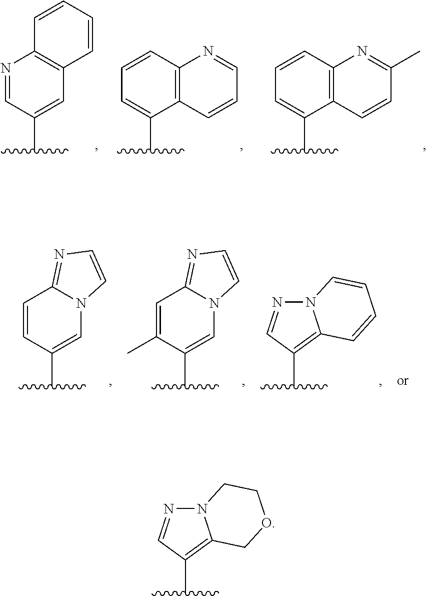

E12.15b. The compound of E12.14b, or a pharmaceutically acceptable salt thereof, wherein G² is

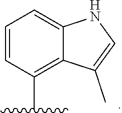 [SMILES: *c1cccc2[nH]cc(C)c12]

E12.16. The compound of E12, or a pharmaceutically acceptable salt thereof, wherein G² is the optionally substituted 4- to 12-membered heterocyclyl.

E12.17. The compound of E12.16, or a pharmaceutically acceptable salt thereof, wherein the optionally substituted 4- to 12-membered heterocyclyl attaches to the parent molecular moiety at a nitrogen ring atom in the 4- to 12-membered heterocyclyl.

E12.18a. The compound of any of E12, E12.16, or E12.17, or a pharmaceutically acceptable salt thereof, wherein the ring system of the 4- to 12-membered heterocyclcyl at G² is pyrrolidin-1-yl, isothiazolidin-2-yl, oxazolidin-3-yl, morpholino, thiomorpholino, 2-oxa-5-azabicyclo[2.2.1]heptan-5-yl, 8-oxa-3-azabicyclo[3.2.1]octan-3-yl, 2-oxa-7-azaspiro[3.5]nonan-7-yl, 6,7-dihydropyrazolo[1,5-a]pyrimidin-4(5H)-yl, 2,3-dihydro-1H-pyrido[2,3-b][1,4]oxazin-1-yl, 2,3-dihydro-4H-benzo[b][1,4]oxazin-4-yl, 2,3-dihydro-1H-pyrrolo[3,2-b]pyridin-1-yl, or indolin-1-yl.

E12.18b. The compound of any of E12, E12.16, or E12.17, or a pharmaceutically acceptable salt thereof, wherein the ring system of the 4- to 12-membered heterocyclcyl at G² is 2-oxa-6-azaspiro[3.3]heptan-6-yl or oxazolidin-3-yl.

E12.19a. The compound of E12.16 or E12.17, or a pharmaceutically acceptable salt thereof, wherein G² is

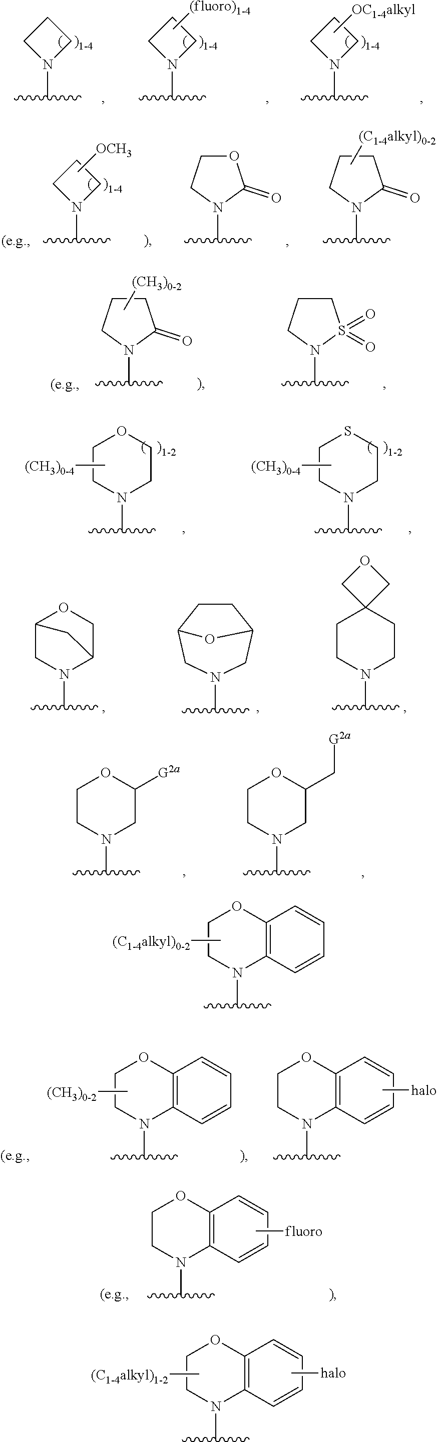

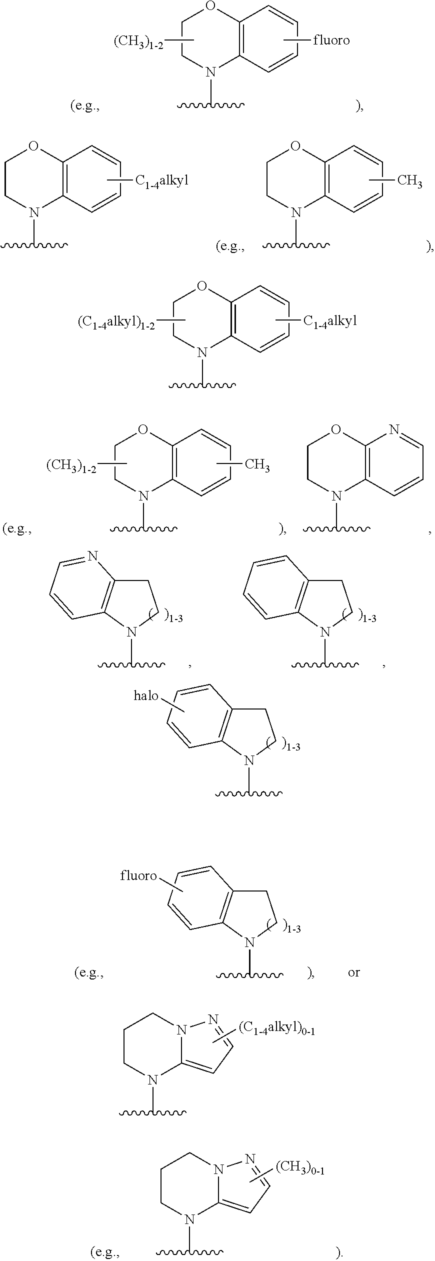

E12.19b. The compound of E12.16 or E12.17, or a pharmaceutically acceptable salt thereof, wherein G² is

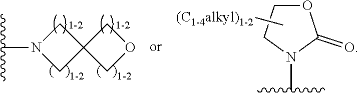

E12.20a. The compound of E12.19a, or a pharmaceutically acceptable salt thereof, wherein G² is

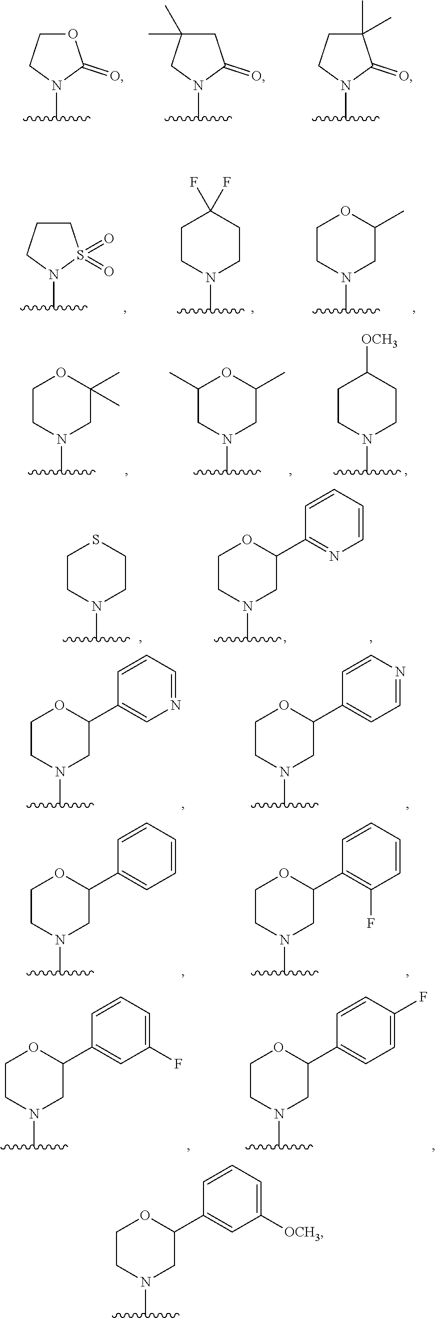

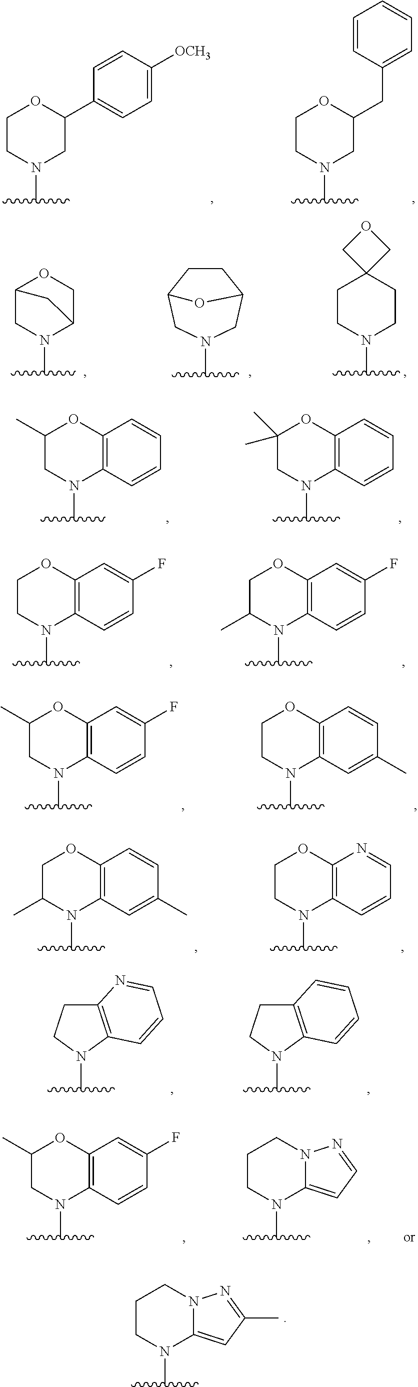

E12.20b. The compound of E12.19b, or a pharmaceutically acceptable salt thereof, wherein G² is

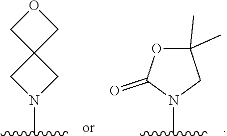

E12.21. The compound of E12.16, or a pharmaceutically acceptable salt thereof, wherein the optionally substituted 4- to 12-membered heterocyclyl attaches to the parent molecular moiety at a carbon ring atom in the 4- to 12-membered heterocyclcyl.

E12.22. The compound of E12, E12.16 or E12.21, or a pharmaceutically acceptable salt thereof, wherein the ring system of the 4- to 12-membered heterocyclyl at G² is a 5- to 7-membered heterocyclyl containing one heteratom selected from the group consisting of O, N, and S.

E12.23. The compound of E12.22, or a pharmaceutically acceptable salt thereof, wherein the ring system of the 5- to 7-membered heterocyclyl at G² is tetrahydropyranyl, 1,2,3,6-tetrahydropyridinyl, 1,2-dihydropyridinyl, or 1,6-dihydropyridinyl, each attached at a ring carbon atom.

E12.24. The compound of E12.23, or a pharmaceutically acceptable salt thereof, wherein the ring system of the 5- to 7-membered heterocyclyl at G² is tetrahydropyran-4-yl, 1,2,3,6-tetrahydropyridin-4-yl, 1,2-dihydropyridin-4-yl, or 1,6-dihydropyridin-3-yl.

E12.25. The compound of E12.22, or a pharmaceutically acceptable salt thereof, wherein G² is

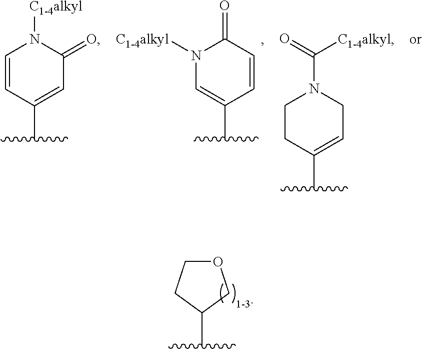

E12.26. The compound of E12.25, or a pharmaceutically acceptable salt thereof, wherein G² is

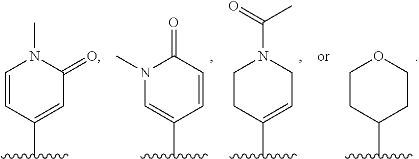

E12.27. The compound of E12, or a pharmaceutically acceptable salt thereof, wherein G² is the optionally substituted 3- to 12-membered carbocyclyl.

E12.28. The compound of E12 or E12.27, or a pharmaceutically acceptable salt thereof, wherein the ring system of the 3- to 12-membered carbocyclyl at G² is C₃₋₆cycloalkyl.

E12.29. The compound of E12.28, or a pharmaceutically acceptable salt thereof, wherein G² is C₃₋₆cycloalkyl.

E12.30. The compound of E12.29, or a pharmaceutically acceptable salt thereof, wherein G² is cyclopropyl or cyclohexyl.

E12.31. The compound of any ofE12.1-E12.2, E12.5-E12.8, or E12.11-E12.13b, or a pharmaceutically acceptable salt thereof, wherein G² is optionally substituted with a first substituent selected from the group consisting of halogen, cyano, C₁₋₆alkyl, C₁₋₆haloalkyl, —OR^{x}, —N(R^{x})₂, —C(O)R^{x}, —C(O)OR^{x}, —C(O)N(R^{x})₂, —C₁₋₆alkylene-OR^{x}, —C₁₋₆alkylene-N(R^{x})₂, G^{2a}, and —C₁₋₃alkylene-G^{2a}; and further optionally substituted with 1-3 substituents independently selected from the group consisting of halogen, C₁₋₄alkyl, C₁₋₄fluoroalkyl, and —OC₁₋₄alkyl.

E12.32. The compound of E12.31, or a pharmaceutically acceptable salt thereof, wherein the optional first G² substituent is selected from the group consisting of fluoro, chloro, cyano, methyl, ethyl, isopropyl, CHF₂, CF₃, CH₂CF₃, —OH, —OCH₃, —OCH(CH₃)₂, —OCHF₂, —OCF₃, NH₂, —N(CH₃)₂, —C(O)H, —C(O)CH₃, COOH, —C(O)NH-cyclopropyl, —CH₂OH, —C(CH₃)₂OH, —CH₂CH₂—N(CH₃)₂, G^{2a}, and —C₁₋₃alkylene-G^{2a}, and the 1-3 further optional substituents are independently selected from the group consisting of methyl, ethyl, fluoro, chloro, CHF₂, CF₃, and —OCH₃.

E12.33. The compound of any of E12.5-E12.8 or E12.31, or a pharmaceutically acceptable salt thereof, wherein G² is

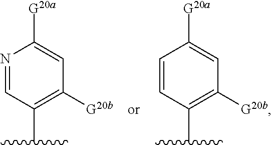

wherein:
G^{20a} is selected from the group consisting of hydrogen, halogen, cyano, C₁₋₄alkyl, C₁₋₂ fluoroalkyl, —OR^{x}, —N(R^{x})₂, and C₃₋₄cycloalkyl;
G^{20b} is selected from the group consisting of halogen, cyano, C₁₋₄alkyl, C₁₋₂fluoroalkyl, —OR^{x}, —N(R^{x})₂, and C₃₋₄cycloalkyl; and R^{x} is C₁₋₃alkyl or C₁₋₂fluoroalkyl.

E12.34. The compound of E12.33, or a pharmaceutically acceptable salt thereof, wherein G^{20a} is hydrogen, halogen, or —OR^{x}.

E12.35. The compound of E12.34, or a pharmaceutically acceptable salt thereof, wherein G^{20a} is hydrogen, fluoro, or —OCH₃.

E12.36. The compound of any of E12.33-E12.35, or a pharmaceutically acceptable salt thereof, wherein G^{20b} is C₁₋₄alkyl.

E12.37. The compound of E12.36, or a pharmaceutically acceptable salt thereof, wherein G^{20b} is methyl.

E12.38. The compound of any of E12.16-E12.18b or E12.21-E12.24, or a pharmaceutically acceptable salt thereof, wherein G² is optionally substituted with 1-5 substituents independently selected from the group consisting of halogen, cyano, C₁₋₆alkyl, C₁₋₆ haloalkyl, oxo, —OR^{x}, —N(R^{x})₂, —C(O)R^{x}, —C(O)OR^{x}, —C(O)N(R^{x})₂, —C₁₋₆alkylene-OR^{x}, —C₁₋₆ alkylene —N(R^{x})₂, G^{2a}, and —C₁₋₃alkylene-G^{2a}.

E12.39. The compound of E12.38, or a pharmaceutically acceptable salt thereof, wherein G² is optionally substituted with a first substituent selected from the group consisting of halogen, cyano, C₁₋₆alkyl, C₁₋₆haloalkyl, oxo, —OR^{x}, —N(R^{x})₂, —C(O)R^{x}, —C(O)OR^{x}, —C(O)N(R^{x})₂, —C₁₋₆alkylene-OR^{x}, —C₁₋₆alkylene-N(R^{x})₂, G^{2a}, and —C₁₋₃alkylene-G^{2a}; and further optionally substituted with 1-3 substituents independently selected from the group consisting of halogen, C₁₋₄alkyl, C₁₋₄fluoroalkyl, and —OC₁₋₄alkyl.

E12.40. The compound of E12.39, or a pharmaceutically acceptable salt thereof, wherein the optional first substituent is selected from the group consisting of halogen, C₁₋₆alkyl, oxo, —OR^{x}, —C(O)R^{x}, G^{2a}, and —CH₂-G^{2a}, and the 1-3 further optional substituents are independently selected from the group consisting of halogen and C₁₋₄alkyl.

E12.41. The compound of any of E12-E12.2, E12.5-E12.8, E12.11-E12.13b, E12.16-E12.18b, E12.21-E12.24, E12.27-E12.28, E12.31-E12.32, or E12.38-E12.40, or a pharmaceutically acceptable salt thereof, wherein R^{x} is hydrogen, C₁₋₄alkyl, C₁₋₄fluoroalkyl, C₃₋₄ cycloalkyl, or —CH₂—C₃₋₄cycloalkyl.

E12.42. The compound of E12.41, or a pharmaceutically acceptable salt thereof, wherein R^{x} is hydrogen, methyl, ethyl, isopropyl, CHF₂, CF₃, or cyclopropyl.

E12.43. The compound of any of E12-E12.3a, E12.5-E12.9, E12.11-E12.13b, E12.16-E12.19a, E12.21-E12.24, E12.27-E12.28, E12.31-E12.32, or E12.38-E12.42, or a pharmaceutically acceptable salt thereof, wherein G^{2a} is the optionally substituted phenyl.

E12.44. The compound of any of E12-E12.3a, E12.5-E12.9, E12.11-E12.13b, E12.16-E12.19a, E12.21-E12.24, E12.27-E12.28, E12.31-E12.32, or E12.38-E12.42, or a pharmaceutically acceptable salt thereof, wherein G^{2a} is the optionally substituted 5- to 6-membered heteroaryl.

E12.45. The compound of any of E12-E12.3a, E12.5-E12.9, E12.11-E12.13b, E12.16-E12.19a, E12.21-E12.24, E12.27-E12.28, E12.31-E12.32, or E12.38-E12.42, or a pharmaceutically acceptable salt thereof, wherein G^{2a} is the optionally substituted 4- to 8-membered heterocyclyl.

E12.46. The compound of any of E12-E12.3a, E12.5-E12.9, E12.11-E12.13b, E12.16-E12.19a, E12.21-E12.24, E12.27-E12.28, E12.31-E12.32, or E12.38-E12.42, or a pharmaceutically acceptable salt thereof, wherein G^{2a} is the optionally substituted 3- to 8-membered carbocyclyl.

E12.47. The compound of any of E12-E12.3a, E12.5-E12.9, E12.11-E12.13b, E12.16-E12.19a, E12.21-E12.24, E12.27-E12.28, E12.31-E12.32, or E12.38-E12.46, or a pharmaceutically acceptable salt thereof, wherein G^{2a} is optionally substituted with 1-2 substituents independently selected from the group consisting of halogen, cyano, C₁₋₄alkyl, C₁₋₄fluoroalkyl, and —OC₁₋₄alkyl.

E12.48. The compound of any of E12-E12.3a, E12.5-E12.9, E12.11-E12.13b, E12.16-E12.19a, E12.21-E12.24, E12.27-E12.28, E12.31-E12.32, or E12.38-E12.46, or a pharmaceutically acceptable salt thereof, wherein G^{2a} is unsubstituted.

E12.49. The compound of any of E12-E12.3a, E12.5-E12.9, E12.11-E12.13b, E12.16-E12.19a, E12.21-E12.24, E12.27-E12.28, E12.31-E12.32, or E12.38-E12.42, or a pharmaceutically acceptable salt thereof, wherein G^{2a} is imidazolyl, pyridinyl, or phenyl optionally substituted with halogen or OCH₃.

E12.50. The compound of any of E12-E12.3a, E12.5-E12.9, E12.11-E12.13b, E12.16-E12.19a, E12.21-E12.24, E12.27-E12.28, E12.31-E12.32, or E12.38-E12.42, or a pharmaceutically acceptable salt thereof, wherein G^{2a} is morpholino, piperidinyl, oxetanyl, or tetrahydropyranyl.

E12.51. The compound of any of E12-E12.3a, E12.5-E12.9, E12.11-E12.13b, E12.16-E12.19a, E12.21-E12.24, E12.27-E12.28, E12.31-E12.32, or E12.38-E12.42, or a pharmaceutically acceptable salt thereof, wherein G^{2a} is C₃₋₄cycloalkyl (e.g., cyclopropyl).

E12.52. The compound of any of E12-E12.3a, E12.5-E12.9, E12.11-E12.13b, E12.16-E12.19a, E12.21-E12.24, E12.27-E12.28, E12.31-E12.32, or E12.38-E12.42, or a pharmaceutically acceptable salt thereof, wherein G^{2a} is

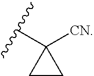 [SMILES: *C1(C#N)CC1]

E12.53. The compound of E12 or E12.1, or a pharmaceutically acceptable salt thereof, wherein the ring system of the optionally substituted 6- to 12-membered aryl at G² is a 9- to 10-membered aryl.

E12.54. The compound of E12.53, or a pharmaceutically acceptable salt thereof, wherein the ring system of the optionally substituted 9- to 10-membered aryl is a phenyl fused to a 5- to 6-membered carbocycle.

12.55. The compound of E12.54, or a pharmaceutically acceptable salt thereof, wherein G² is indan-4-yl

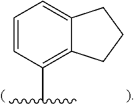 [SMILES: *c1cccc2c1CCC2]

E13. The compound of E4, or a pharmaceutically acceptable salt thereof, wherein R² is —C₁₋₃alkylene-G².

E13.1. The compound of E13, or a pharmaceutically acceptable salt thereof, wherein R² is —CH₂-G².

E13.2. The compound of E13 or E13.1, or a pharmaceutically acceptable salt thereof, wherein G² is pyridinyl.

E13.3. The compound of E13.2, or a pharmaceutically acceptable salt thereof, wherein G² is pyridin-3-yl.

E14. The compound of any of E1-E13.3, or a pharmaceutically acceptable salt thereof, wherein R⁴ is hydrogen, halogen, C₁₋₄alkyl, C₂₋₄alkenyl, or G⁴.

E14.1. The compound of any of E1-E14, or a pharmaceutically acceptable salt thereof, wherein G⁴ is phenyl, a 4- to 7-membered heterocyclyl containing 1-2 heteroatoms independently selected from the group consisting of O, N, and S, a C₃₋₆cycloalkyl, or a C₅₋₆cycloalkenyl.

E14.2. The compound of E14.1, or a pharmaceutically acceptable salt thereof, wherein R⁴ is hydrogen, bromo, chloro, fluoro, methyl, ethyl, vinyl, morpholino, cyclopentyl, cyclopentenyl, or phenyl.

E14.3. The compound of E14.2, or a pharmaceutically acceptable salt thereof, wherein R⁴ is hydrogen.

E15. The compound of any of E1-E14.3, or a pharmaceutically acceptable salt thereof, wherein R⁵ is hydrogen, C₁₋₄alkyl, C₁₋₄fluoroalkyl, —C₁₋₃alkylene-OR^{5a}, —C(O)NR^{5a}R^{5b}C₃₋₆cycloalkyl, or —C₁₋₃alkylene-C₃₋₆cycloalkyl.

E15.1. The compound of any of E1-E15, or a pharmaceutically acceptable salt thereof, wherein R^{5a} is hydrogen or C₁₋₄alkyl.

E15.1a. The compound of any of E1-E15.1, or a pharmaceutically acceptable salt thereof, wherein R^{5a} is hydrogen.

E15.2. The compound of any of E1-E15.1a, or a pharmaceutically acceptable salt thereof, wherein R^{5b} is G⁵ or —C₁₋₃alkylene-G⁵.

E15.3. The compound of any of E1-E15.2, or a pharmaceutically acceptable salt thereof, wherein G⁵ is the 5- to 6-membered heteroaryl, the 4- to 8-membered heterocyclyl, or the C₃₋₆cycloalkyl, wherein G⁵ is optionally substituted with 1-4 substituents independently selected from the group consisting of halogen, cyano, C₁₋₄alkyl, and C₁₋₄fluoroalkyl.

E15.4. The compound of any of E1-E15.3, or a pharmaceutically acceptable salt thereof, wherein G⁵ is thiazolyl, azetidinyl, or cyclopropyl, each optionally substituted with 1-2 substituents independently selected from the group consisting of halogen, cyano, C₁₋₄alkyl, and C₁₋₄fluoroalkyl.

E15.5. The compound of any of E1-E15.4, or a pharmaceutically acceptable salt thereof, wherein G⁵ is optionally substituted with cyano or C₁₋₂fluoroalkyl (e.g., CH₂CF₃).

E15.6. The compound of E15 or E15.1, or a pharmaceutically acceptable salt thereof, wherein R⁵ is hydrogen, C₁₋₄alkyl, or —C₁₋₃alkylene-OR^{5a}.

E15.7. The compound of E15.6, or a pharmaceutically acceptable salt thereof, wherein R⁵ is hydrogen, methyl, or —CH₂OCH₃.

E15.8. The compound of any of E1-E15, or a pharmaceutically acceptable salt thereof, wherein R^{5a} and R^{5b}, together with the nitrogen to which they attach form the optionally substituted 4- to 10-membered heterocyclic ring.

E15.9. The compound of E15.8, or a pharmaceutically acceptale salt thereof, wherein R^{5a} and R^{5b}, together with the nitrogen to which they attach form a 4- to 7-membered monocyclic heterocyclyl or a 7- to 10-membered spirocyclic heterocyclyl, wherein the heterocyclyls optionally contain 1 additional heteroatom that is O, N, or S and the monocyclic heterocyclyl is optionally substituted with a first substituent selected from the group consisting of C₁₋₄alkyl, halogen, —OC₁₋₄alkyl, and pyridinyl and optionally further substituted with 1-3 substituents that are independently halogen or C₁₋₄alkyl.

E15.10. The compound of E15.9, or a pharmaceutically acceptable salt thereof, wherein the first optional substituent is methyl, fluoro, —OCH₃, or pyridinyl and the 1-3 additional optional substituents are independently methyl or fluoro.

E15.11. The compound of any of E15.8-E15.10, or a pharmaceutically acceptable salt thereof, wherein R^{5a} and R^{5b}, together with the nitrogen to which they attach form

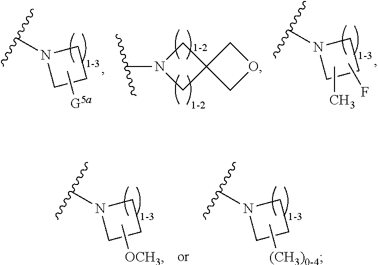

and G⁵a is pyridinyl.

E15.12. The compound of E15.11, or a pharmaceutically acceptable salt thereof, wherein R^{5a} and R^{5b}, together with the nitrogen to which they attach form

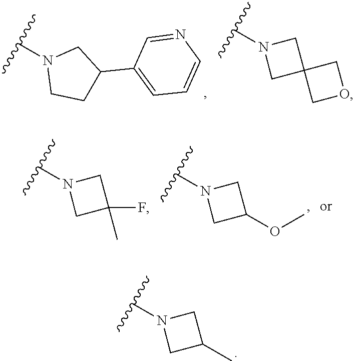

E15.13. The compound of any of E1-E15, or a pharmaceutically acceptable salt thereof, wherein R⁵ is hydrogen, methyl, ethyl, isopropyl, CF₃, CHF₂, —CH₂OCH₃, —C(CH₃)₂OH, cyclopropyl, —CH₂cyclopentyl

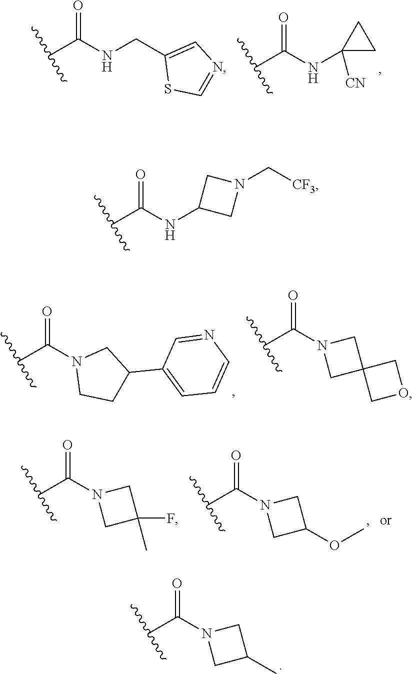

E16. The compound of any of E1-E15.13, or a pharmaceutically acceptable salt thereof, wherein R⁶ is hydrogen, C₁₋₄alkyl, —C₁₋₃alkylene-OR^{6a}, or C₃₋₆cycloalkyl.

E16.1. The compound of E16, or a pharmaceutically acceptable salt thereof, wherein R⁶ is hydrogen, methyl, CH₂OCH₃, or cyclopropyl.

E16.2. The compound of E16.1, or a pharmaceutically acceptable salt thereof, wherein R⁶ is hydrogen or C₁₋₄alkyl.

E16.3. The compound of E16.2, or a pharmaceutically acceptable salt thereof, wherein R⁶ is hydrogen or methyl.

E17. The compound of any of E1-E16.3, or a pharmaceutically acceptable salt thereof, wherein R⁷ is hydrogen, C₁₋₄alkyl, —C₁₋₃alkylene-OR^{7a}, or C₃₋₆cycloalkyl.

E17.1. The compound of E17, or a pharmaceutically acceptable salt thereof, wherein R⁷ is hydrogen, methyl, CH₂OCH₃, or cyclopropyl.

E17.2. The compound of E17, or a pharmaceutically acceptable salt thereof, wherein R⁷ is C₁₋₄alkyl.

E17.3. The compound of any of E17-E17.2, or a pharmaceutically acceptable salt thereof, wherein R⁷ is methyl.

E18. The compound of any of E1-E17.3, or a pharmaceutically acceptable salt thereof, wherein n is 0 or 1.

E18.1. The compound of E18, or a pharmaceutically acceptable salt thereof, wherein n is 1.

E18.2. The compound of E18.1, or a pharmaceutically acceptable salt thereof, wherein the compound has formula (I-A):

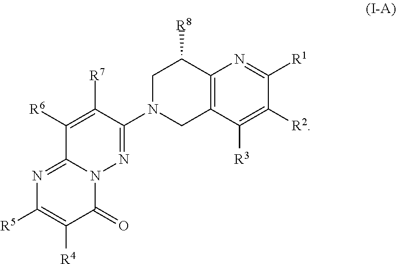

E18.3. The compound of E18.1, or a pharmaceutically acceptable salt thereof, wherein the compound has formula (I-B):

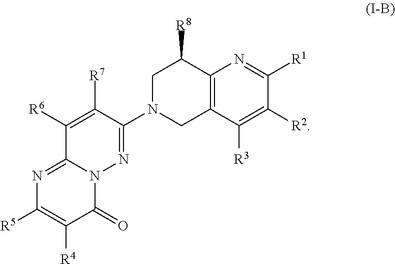

E18.4. The compound of E18.1, or a pharmaceutically acceptable salt thereof, wherein the compound has formula (I-C):

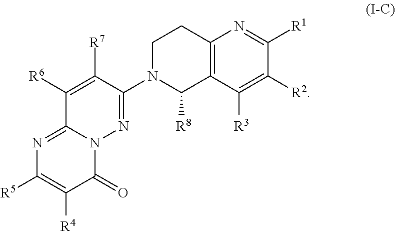

E18.5. The compound of E18.1, or a pharmaceutically acceptable salt thereof, wherein the compound has formula (I-D):

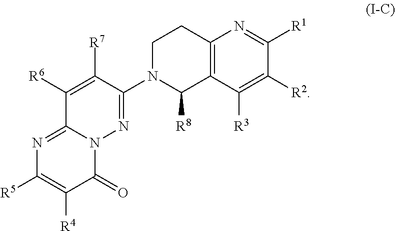

E18.6. The compound of E18, or a pharmaceutically acceptable salt thereof, wherein n is 0 (i.e.,

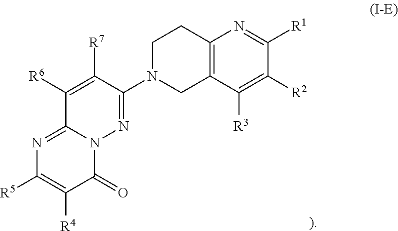

E18.7. The compound of E18.6, or a pharmaceutically acceptable salt thereof, wherein the compound has formula (I-F):

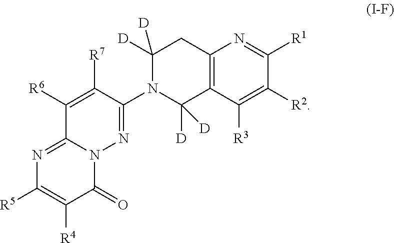

E19. The compound of any of E1-E18.5, or a pharmaceutically acceptable salt thereof, wherein R⁸ is C₁₋₄alkyl.

E19.1. The compound of E19, or a pharmaceutically acceptable salt thereof, wherein R⁸ is methyl.

E20. The compound of E1 selected from the group consisting of

8-methyl-7-(3-(trifluoromethyl)-7,8-dihydro-1,6-naphthyridin-6(5H)-yl)-4H-pyrimido[1,2-
b]pyridazin-4-one
7-(3-(1,3-dimethyl-1H-pyrazol-4-yl)-7,8-dihydro-1,6-naphthyridin-6(5H)-yl)-8-methyl-4H-
pyrimido[1,2-b]pyridazin-4-one
8-methyl-7-(3-(6-methylpyridin-3-yl)-7,8-dihydro-1,6-naphthyridin-6(5H)-yl)-4H-
pyrimido[1,2-b]pyridazin-4-one
3-chloro-8-methyl-7-(3-(trifluoromethyl)-7,8-dihydro-1,6-naphthyridin-6(5H)-yl)-4H-
pyrimido[1,2-b]pyridazin-4-one
8-methyl-7-(3-(2-methylthiazol-5-yl)-7,8-dihydro-1,6-naphthyridin-6(5H)-yl)-4H-
pyrimido[1,2-b]pyridazin-4-one
8-methyl-7-(3-(1-methyl-1H-pyrazol-4-yl)-7,8-dihydro-1,6-naphthyridin-6(5H)-yl)-4H-
pyrimido[1,2-b]pyridazin-4-one
8-methyl-7-(3-(1-methyl-1H-pyrazol-5-yl)-7,8-dihydro-1,6-naphthyridin-6(5H)-yl)-4H-
pyrimido[1,2-b]pyridazin-4-one
7-(3-(1-cyclopropyl-1H-pyrazol-4-yl)-7,8-dihydro-1,6-naphthyridin-6(5H)-yl)-8-methyl-
4H-pyrimido[1,2-b]pyridazin-4-one
3-chloro-7-(3-cyclopropyl-7,8-dihydro-1,6-naphthyridin-6(5H)-yl)-8-methyl-4H-
pyrimido[1,2-b]pyridazin-4-one
3-chloro-8-methyl-7-(3-(1-methyl-1H-pyrazol-5-yl)-7,8-dihydro-1,6-naphthyridin-6(5H)-
yl)-4H-pyrimido[1,2-b]pyridazin-4-one
3-chloro-7-(3-(1,3-dimethyl-1H-pyrazol-4-yl)-7,8-dihydro-1,6-naphthyridin-6(5/)-yl)-8-
methyl-4H-pyrimido[1,2-b]pyridazin-4-one
3-chloro-7-(3-(1-cyclopropyl-1H-pyrazol-4-yl)-7,8-dihydro-1,6-naphthyridin-6(5H)-yl)-8-
methyl-4H-pyrimido[1,2-b]pyridazin-4-one
3-chloro-8-methyl-7-(3-(2-methylthiazol-5-yl)-7,8-dihydro-1,6-naphthyridin-6(5H)-yl)-4H-
pyrimido[1,2-b]pyridazin-4-one
3-chloro-8-methyl-7-(3-(6-methylpyridin-3-yl)-7,8-dihydro-1,6-naphthyridin-6(5H)-yl)-4H-
pyrimido[1,2-b]pyridazin-4-one
3-fluoro-2,8-dimethyl-7-(3-(trifluoromethyl)-7,8-dihydro-1,6-naphthyridin-6(5H)-yl)-4H-
pyrimido[1,2-b]pyridazin-4-one
2,8-dimethyl-7-(3-(trifluoromethyl)-7,8-dihydro-1,6-naphthyridin-6(5H)-yl)-4H-
pyrimido[1,2-b]pyridazin-4-one
8-methyl-2-(trifluoromethyl)-7-(3-(trifluoromethyl)-7,8-dihydro-1,6-naphthyridin-6(5H)-
yl)-4H-pyrimido[1,2-b]pyridazin-4-one
3-fluoro-2,8,9-trimethyl-7-(3-(trifluoromethyl)-7,8-dihydro-1,6-naphthyridin-6(5H)-yl)-4H-
pyrimido[1,2-b]pyridazin-4-one
8-methyl-3-morpholino-7-(3-(trifluoromethyl)-7,8-dihydro-1,6-naphthyridin-6(5H)-yl)-4H-
pyrimido[1,2-b]pyridazin-4-one
3-(cyclopent-1-en-1-yl)-8-methyl-7-(3-(trifluoromethyl)-7,8-dihydro-1,6-naphthyridin-
6(5H)-yl)-4H-pyrimido[1,2-b]pyridazin-4-one
8-methyl-3-phenyl-7-(3-(trifluoromethyl)-7,8-dihydro-1,6-naphthyridin-6(5H)-yl)-4H-
pyrimido[1,2-b]pyridazin-4-one
8-methyl-7-(3-(trifluoromethyl)-7,8-dihydro-1,6-naphthyridin-6(5H)-yl)-3-vinyl-4H-
pyrimido[1,2-b]pyridazin-4-one
3-cyclopentyl-8-methyl-7-(3-(trifluoromethyl)-7,8-dihydro-1,6-naphthyridin-6(5H)-yl)-4H-
pyrimido[1,2-b]pyridazin-4-one
3-ethyl-8-methyl-7-(3-(trifluoromethyl)-7,8-dihydro-1,6-naphthyridin-6(5H)-yl)-4H-
pyrimido[1,2-b]pyridazin-4-one
2,3,8-trimethyl-7-(3-(trifluoromethyl)-7,8-dihydro-1,6-naphthyridin-6(5H)-yl)-4H-
pyrimido[1,2-b]pyridazin-4-one
2-(difluoromethyl)-8-methyl-7-(3-(trifluoromethyl)-7,8-dihydro-1,6-naphthyridin-6(5H)-
yl)-4H-pyrimido[1,2-b]pyridazin-4-one
2-cyclopropyl-8-methyl-7-(3-(trifluoromethyl)-7,8-dihydro-1,6-naphthyridin-6(5H)-yl)-4H-
pyrimido[1,2-b]pyridazin-4-one
3-chloro-2,8-dimethyl-7-(3-(trifluoromethyl)-7,8-dihydro-1,6-naphthyridin-6(5H)-yl)-4H-
pyrimido[1,2-b]pyridazin-4-one
2,8-dimethyl-7-(3-(1-methyl-1H-pyrazol-5-yl)-7,8-dihydro-1,6-naphthyridin-6(5H)-yl)-4H-
pyrimido[1,2-b]pyridazin-4-one
2,8-dimethyl-7-(3-(6-methylpyridin-3-yl)-7,8-dihydro-1,6-naphthyridin-6(5H)-yl)-4H-
pyrimido[1,2-b]pyridazin-4-one
2,8-dimethyl-7-(3-(2-methylthiazol-5-yl)-7,8-dihydro-1,6-naphthyridin-6(5H)-yl)-4H-
pyrimido[1,2-b]pyridazin-4-one
7-(3-(1-cyclopropyl-1H-pyrazol-4-yl)-7,8-dihydro-1,6-naphthyridin-6(5H)-yl)-2,8-
dimethyl-4H-pyrimido[1,2-b]pyridazin-4-one
2,8,9-trimethyl-7-(3-(trifluoromethyl)-7,8-dihydro-1,6-naphthyridin-6(5H)-yl)-4H-
pyrimido[1,2-b]pyridazin-4-one
2-(difluoromethyl)-7-(3-(1,3-dimethyl-1H-pyrazol-4-yl)-7,8-dihydro-1,6-naphthyridin-
6(5H)-yl)-8-methyl-4H-pyrimido[1,2-b]pyridazin-4-one
2-(difluoromethyl)-8-methyl-7-(3-(1-methyl-1H-pyrazol-4-yl)-7,8-dihydro-1,6-
naphthyridin-6(5H)-yl)-4H-pyrimido[1,2-b]pyridazin-4-one
2-(difluoromethyl)-8-methyl-7-(3-(1-methyl-1H-pyrazol-5-yl)-7,8-dihydro-1,6-
naphthyridin-6(5H)-yl)-4H-pyrimido[1,2-b]pyridazin-4-one
8-methyl-7-(3-(1-methyl-2-oxo-1,2-dihydropyridin-4-yl)-7,8-dihydro-1,6-naphthyridin-
6(5H)-yl)-4H-pyrimido[1,2-b]pyridazin-4-one
7-(3-(2-methoxypyridin-4-yl)-7,8-dihydro-1,6-naphthyridin-6(5H)-yl)-8-methyl-4H-
pyrimido[1,2-b]pyridazin-4-one
8,9-dimethyl-2-(trifluoromethyl)-7-(3-(trifluoromethyl)-7,8-dihydro-1,6-naphthyridin-
6(5H)-yl)-4H-pyrimido[1,2-b]pyridazin-4-one
2-(difluoromethyl)-8,9-dimethyl-7-(3-(trifluoromethyl)-7,8-dihydro-1,6-naphthyridin-
6(5H)-yl)-4H-pyrimido[1,2-b]pyridazin-4-one
8-methyl-7-(3-(quinolin-5-yl)-7,8-dihydro-1,6-naphthyridin-6(5H)-yl)-4H-pyrimido[1,2-
b]pyridazin-4-one
8-methyl-4-oxo-N-(thiazol-5-ylmethyl)-7-(3-(trifluoromethyl)-7,8-dihydro-1,6-
naphthyridin-6(5H)-yl)-4H-pyrimido[1,2-b]pyridazine-2-carboxamide
8-methyl-2-(3-(pyridin-3-yl)pyrrolidine-1-carbonyl)-7-(3-(trifluoromethyl)-7,8-dihydro-1,6-
naphthyridin-6(5H)-yl)-4H-pyrimido[1,2-b]pyridazin-4-one
8-methyl-2-(2-oxa-6-azaspiro[3.3]heptane-6-carbonyl)-7-(3-(trifluoromethyl)-7,8-dihydro-
1,6-naphthyridin-6(5H)-yl)-4H-pyrimido[1,2-b]pyridazin-4-one
2-(3-fluoro-3-methylazetidine-1-carbonyl)-8-methyl-7-(3-(trifluoromethyl)-7,8-dihydro-
1,6-naphthyridin-6(5H)-yl)-4H-pyrimido[1,2-b]pyridazin-4-one
N-(1-cyanocyclopropyl)-8-methyl-4-oxo-7-(3-(trifluoromethyl)-7,8-dihydro-1,6-
naphthyridin-6(5H)-yl)-4H-pyrimido[1,2-b]pyridazine-2-carboxamide
2-(3-methoxyazetidine-1-carbonyl)-8-methyl-7-(3-(trifluoromethyl)-7,8-dihydro-1,6-
naphthyridin-6(5H)-yl)-4H-pyrimido[1,2-b]pyridazin-4-one
8-methyl-7-(3-(2-methylquinolin-5-yl)-7,8-dihydro-1,6-naphthyridin-6(5H)-yl)-4H-
pyrimido[1,2-b]pyridazin-4-one
8-methyl-7-(3-(1-methyl-6-oxo-1,6-dihydropyridin-3-yl)-7,8-dihydro-1,6-naphthyridin-
6(5H)-yl)-4H-pyrimido[1,2-b]pyridazin-4-one
7-(3-(3-methoxypyridin-4-yl)-7,8-dihydro-1,6-naphthyridin-6(5H)-yl)-8-methyl-4H-
pyrimido[1,2-b]pyridazin-4-one
7-(3-(4-fluoro-3-methoxyphenyl)-7,8-dihydro-1,6-naphthyridin-6(5H)-yl)-8-methyl-4H-
pyrimido[1,2-b]pyridazin-4-one
7-(3-(5-methoxypyridin-3-yl)-7,8-dihydro-1,6-naphthyridin-6(5H)-yl)-8-methyl-4H-
pyrimido[1,2-b]pyridazin-4-one
7-(3-(imidazo[1,2-a]pyridin-6-yl)-7,8-dihydro-1,6-naphthyridin-6(5H)-yl)-8-methyl-4H-
pyrimido[1,2-b]pyridazin-4-one
7-(3-(2-cyclopropylpyridin-4-yl)-7,8-dihydro-1,6-naphthyridin-6(5H)-yl)-8-methyl-4H-
pyrimido[1,2-b]pyridazin-4-one
8-methyl-4-oxo-N-(1-(2,2,2-trifluoroethyl)azetidin-3-yl)-7-(3-(trifluoromethyl)-7,8-
dihydro-1,6-naphthyridin-6(5H)-yl)-4H-pyrimido[1,2-b]pyridazine-2-carboxamide
8-methyl-2-(3-methylazetidine-1-carbonyl)-7-(3-(trifluoromethyl)-7,8-dihydro-1,6-
naphthyridin-6(5H)-yl)-4H-pyrimido[1,2-b]pyridazin-4-one
7-(3-(5-chloro-2-methoxypyridin-4-yl)-7,8-dihydro-1,6-naphthyridin-6(5H)-yl)-8-methyl-
4H-pyrimido[1,2-b]pyridazin-4-one
7-(3-(2-fluoropyridin-3-yl)-7,8-dihydro-1,6-naphthyridin-6(5H)-yl)-8-methyl-4H-
pyrimido[1,2-b]pyridazin-4-one
7-(3-(3-chloro-4-fluorophenyl)-7,8-dihydro-1,6-naphthyridin-6(5H)-yl)-8-methyl-4H-
pyrimido[1,2-b]pyridazin-4-one
7-(3-(6-methoxypyridin-3-yl)-7,8-dihydro-1,6-naphthyridin-6(5H)-yl)-8-methyl-4H-
pyrimido[1,2-b]pyridazin-4-one
8,9-dimethyl-7-(3-(6-methylpyridin-3-yl)-7,8-dihydro-1,6-naphthyridin-6(5H)-yl)-4H-
pyrimido[1,2-b]pyridazin-4-one
8,9-dimethyl-7-(3-(1-methyl-1H-pyrazol-4-yl)-7,8-dihydro-1,6-naphthyridin-6(5H)-yl)-4H-
pyrimido[1,2-b]pyridazin-4-one
8,9-dimethyl-7-(3-(1-methyl-1H-pyrazol-5-yl)-7,8-dihydro-1,6-naphthyridin-6(5H)-yl)-4H-
pyrimido[1,2-b]pyridazin-4-one
8,9-dimethyl-7-(3-(trifluoromethyl)-7,8-dihydro-1,6-naphthyridin-6(5H)-yl)-4H-
pyrimido[1,2-b]pyridazin-4-one
8-methyl-7-(3-(5-methylpyridin-3-yl)-7,8-dihydro-1,6-naphthyridin-6(5H)-yl)-4H-
pyrimido[1,2-b]pyridazin-4-one
8-methyl-7-(3-(6-(trifluoromethyl)pyridin-3-yl)-7,8-dihydro-1,6-naphthyridin-6(5H)-yl)-
4H-pyrimido[1,2-b]pyridazin-4-one
7-(3-(6-cyclopropylpyridin-3-yl)-7,8-dihydro-1,6-naphthyridin-6(5H)-yl)-8-methyl-4H-
pyrimido[1,2-b]pyridazin-4-one
8-methyl-7-(3-(pyridin-3-yl)-7,8-dihydro-1,6-naphthyridin-6(5H)-yl)-4H-pyrimido[1,2-
b]pyridazin-4-one
7-(3-(2-fluoropyridin-3-yl)-7,8-dihydro-1,6-naphthyridin-6(5H)-yl)-2,8-dimethyl-4H-
pyrimido[1,2-b]pyridazin-4-one
7-(3-(5-methoxypyridin-3-yl)-7,8-dihydro-1,6-naphthyridin-6(5H)-yl)-2,8-dimethyl-4H-
pyrimido[1,2-b]pyridazin-4-one
7-(3-(6-methoxypyridin-3-yl)-7,8-dihydro-1,6-naphthyridin-6(5H)-yl)-2,8-dimethyl-4H-
pyrimido[1,2-b]pyridazin-4-one
7-(3-(6-cyclopropylpyridin-3-yl)-7,8-dihydro-1,6-naphthyridin-6(5H)-yl)-2,8-dimethyl-4H-
pyrimido[1,2-b]pyridazin-4-one
2,8-dimethyl-7-(3-(6-(trifluoromethyl)pyridin-3-yl)-7,8-dihydro-1,6-naphthyridin-6(5H)-
yl)-4H-pyrimido[1,2-b]pyridazin-4-one
7-(3-(5-methoxypyridin-3-yl)-7,8-dihydro-1,6-naphthyridin-6(5H)-yl)-8-methyl-2-
(trifluoromethyl)-4H-pyrimido[1,2-b]pyridazin-4-one
7-(3-(6-methoxypyridin-3-yl)-7,8-dihydro-1,6-naphthyridin-6(5H)-yl)-8-methyl-2-
(trifluoromethyl)-4H-pyrimido[1,2-b]pyridazin-4-one
N-(1-cyanocyclopropyl)-8-methyl-7-(3-(6-methylpyridin-3-yl)-7,8-dihydro-1,6-
naphthyridin-6(5H)-yl)-4-oxo-4H-pyrimido[1,2-b]pyridazine-2-carboxamide
8-methyl-7-(3-(6-methylpyridin-3-yl)-7,8-dihydro-1,6-naphthyridin-6(5H)-yl)-4-oxo-N-
(thiazol-5-ylmethyl)-4H-pyrimido[1,2-b]pyridazine-2-carboxamide
3-bromo-8-methyl-7-(3-(6-methylpyridin-3-yl)-7,8-dihydro-1,6-naphthyridin-6(5H)-yl)-4H-
pyrimido[1,2-b]pyridazin-4-one
2-cyclopropyl-8-methyl-7-(3-(6-methylpyridin-3-yl)-7,8-dihydro-1,6-naphthyridin-6(5H)-
yl)-4H-pyrimido[1,2-b]pyridazin-4-one
8-methyl-7-(3-(6-methylpyridin-3-yl)-7,8-dihydro-1,6-naphthyridin-6(5H)-yl)-2-
(trifluoromethyl)-4H-pyrimido[1,2-b]pyridazin-4-one
2-(difluoromethyl)-8-methyl-7-(3-(6-methylpyridin-3-yl)-7,8-dihydro-1,6-naphthyridin-
6(5H)-yl)-4H-pyrimido[1,2-b]pyridazin-4-one
2,8,9-trimethyl-7-(3-(6-methylpyridin-3-yl)-7,8-dihydro-1,6-naphthyridin-6(5H)-yl)-4H-
pyrimido[1,2-b]pyridazin-4-one
2,3,8-trimethyl-7-(3-(6-methylpyridin-3-yl)-7,8-dihydro-1,6-naphthyridin-6(5H)-yl)-4H-
pyrimido[1,2-b]pyridazin-4-one
8,9-dimethyl-7-(3-(6-methylpyridin-3-yl)-7,8-dihydro-1,6-naphthyridin-6(5H)-yl)-2-
(trifluoromethyl)-4H-pyrimido[1,2-b]pyridazin-4-one
2-(difluoromethyl)-8,9-dimethyl-7-(3-(6-methylpyridin-3-yl)-7,8-dihydro-1,6-naphthyridin-
6(5H)-yl)-4H-pyrimido[1,2-b]pyridazin-4-one
3,8-dimethyl-7-(3-(6-methylpyridin-3-yl)-7,8-dihydro-1,6-naphthyridin-6(5H)-yl)-4H-
pyrimido[1,2-b]pyridazin-4-one
7-(3-(6-fluoropyridin-3-yl)-7,8-dihydro-1,6-naphthyridin-6(5H)-yl)-8,9-dimethyl-4H-
pyrimido[1,2-b]pyridazin-4-one
3-bromo-8,9-dimethyl-7-(3-(6-methylpyridin-3-yl)-7,8-dihydro-1,6-naphthyridin-6(5H)-yl)-
4H-pyrimido[1,2-b]pyridazin-4-one
7-(3-(6-(dimethylamino)pyridin-3-yl)-7,8-dihydro-1,6-naphthyridin-6(5H)-yl)-8,9-
dimethyl-4H-pyrimido[1,2-b]pyridazin-4-one
8,9-dimethyl-7-(3-(6-(trifluoromethyl)pyridin-3-yl)-7,8-dihydro-1,6-naphthyridin-6(5H)-
yl)-4H-pyrimido[1,2-b]pyridazin-4-one
7-(3-(6-cyclopropylpyridin-3-yl)-7,8-dihydro-1,6-naphthyridin-6(5H)-yl)-8,9-dimethyl-4H-
pyrimido[1,2-b]pyridazin-4-one
2-(difluoromethyl)-7-(3-(6-(dimethylamino)pyridin-3-yl)-7,8-dihydro-1,6-naphthyridin-
6(5H)-yl)-8,9-dimethyl-4H-pyrimido[1,2-b]pyridazin-4-one
2-(difluoromethyl)-8,9-dimethyl-7-(3-(6-(trifluoromethyl)pyridin-3-yl)-7,8-dihydro-1,6-
naphthyridin-6(5H)-yl)-4H-pyrimido[1,2-b]pyridazin-4-one
7-(3-(6-cyclopropylpyridin-3-yl)-7,8-dihydro-1,6-naphthyridin-6(5H)-yl)-2-
(difluoromethyl)-8,9-dimethyl-4H-pyrimido[1,2-b]pyridazin-4-one
8,9-dimethyl-7-(3-(4-methylpyridin-3-yl)-7,8-dihydro-1,6-naphthyridin-6(5H)-yl)-4/-
pyrimido[1,2-b]pyridazin-4-one
7-(3-(4-ethylpyridin-3-yl)-7,8-dihydro-1,6-naphthyridin-6(5H)-yl)-8,9-dimethyl-4HI-
pyrimido[1,2-b]pyridazin-4-one
8,9-dimethyl-7-(3-(6-(piperidin-1-yl)pyridin-3-yl)-7,8-dihydro-1,6-naphthyridin-6(5H)-yl)-
4H-pyrimido[1,2-b]pyridazin-4-one
7-(3-(6-(1H-imidazol-1-yl)pyridin-3-yl)-7,8-dihydro-1,6-naphthyridin-6(5H)-yl)-8,9-
dimethyl-4H-pyrimido[1,2-b]pyridazin-4-one
8,9-dimethyl-7-(3-(6-morpholinopyridin-3-yl)-7,8-dihydro-1,6-naphthyridin-6(5H)-yl)-4H-
pyrimido[1,2-b]pyridazin-4-one
3,8,9-trimethyl-7-(3-(6-methylpyridin-3-yl)-7,8-dihydro-1,6-naphthyridin-6(5H)-yl)-4H-
pyrimido[1,2-b]pyridazin-4-one
8,9-dimethyl-7-(3-(2-methylpyridin-3-yl)-7,8-dihydro-1,6-naphthyridin-6(5H)-yl)-4H-
pyrimido[1,2-b]pyridazin-4-one
8,9-dimethyl-7-(3-(5-(trifluoromethyl)pyridin-3-yl)-7,8-dihydro-1,6-naphthyridin-6(5H)-
yl)-4H-pyrimido[1,2-b]pyridazin-4-one
7-(3-(5-fluoropyridin-3-yl)-7,8-dihydro-1,6-naphthyridin-6(5H)-yl)-8,9-dimethyl-4H-
pyrimido[1,2-b]pyridazin-4-one
8,9-dimethyl-7-(3-(pyrimidin-5-yl)-7,8-dihydro-1,6-naphthyridin-6(5H)-yl)-4H-
pyrimido[1,2-b]pyridazin-4-one
2-ethyl-8,9-dimethyl-7-(3-(trifluoromethyl)-7,8-dihydro-1,6-naphthyridin-6(5H)-yl)-4H-
pyrimido[1,2-b]pyridazin-4-one
2-ethyl-8,9-dimethyl-7-(3-(6-methylpyridin-3-yl)-7,8-dihydro-1,6-naphthyridin-6(5H)-yl)-
4H-pyrimido[1,2-b]pyridazin-4-one
2-isopropyl-8,9-dimethyl-7-(3-(trifluoromethyl)-7,8-dihydro-1,6-naphthyridin-6(5H)-yl)-
4H-pyrimido[1,2-b]pyridazin-4-one
2-isopropyl-8,9-dimethyl-7-(3-(6-methylpyridin-3-yl)-7,8-dihydro-1,6-naphthyridin-6(5H)-
yl)-4H-pyrimido[1,2-b]pyridazin-4-one
2-(methoxymethyl)-8,9-dimethyl-7-(3-(trifluoromethyl)-7,8-dihydro-1,6-naphthyridin-
6(5H)-yl)-4H-pyrimido[1,2-b]pyridazin-4-one
2-(methoxymethyl)-8,9-dimethyl-7-(3-(6-methylpyridin-3-yl)-7,8-dihydro-1,6-
naphthyridin-6(5H)-yl)-4H-pyrimido[1,2-b]pyridazin-4-one
8-cyclopropyl-9-methyl-7-(3-(trifluoromethyl)-7,8-dihydro-1,6-naphthyridin-6(5H)-yl)-4H-
pyrimido[1,2-b]pyridazin-4-one
8-cyclopropyl-9-methyl-7-(3-(6-methylpyridin-3-yl)-7,8-dihydro-1,6-naphthyridin-6(5H)-
yl)-4H-pyrimido[1,2-b]pyridazin-4-one
9-cyclopropyl-8-methyl-7-(3-(trifluoromethyl)-7,8-dihydro-1,6-naphthyridin-6(5H)-yl)-4H-
pyrimido[1,2-b]pyridazin-4-one
9-cyclopropyl-8-methyl-7-(3-(6-methylpyridin-3-yl)-7,8-dihydro-1,6-naphthyridin-6(5H)-
yl)-4H-pyrimido[1,2-b]pyridazin-4-one
2-(difluoromethyl)-8,9-dimethyl-7-(3-(2-methylpyridin-3-yl)-7,8-dihydro-1,6-naphthyridin-
6(5H)-yl)-4H-pyrimido[1,2-b]pyridazin-4-one
2-(difluoromethyl)-8,9-dimethyl-7-(3-(pyrazin-2-yl)-7,8-dihydro-1,6-naphthyridin-6(5H)-
yl)-4H-pyrimido[1,2-b]pyridazin-4-one
2,8,9-trimethyl-7-(3-(4-methylpyridin-3-yl)-7,8-dihydro-1,6-naphthyridin-6(5H)-yl)-4H-
pyrimido[1,2-b]pyridazin-4-one
2,8,9-trimethyl-7-(3-(pyrimidin-5-yl)-7,8-dihydro-1,6-naphthyridin-6(5H)-yl)-4H-
pyrimido[1,2-b]pyridazin-4-one
2-(difluoromethyl)-8,9-dimethyl-7-(3-(5-methylpyridin-3-yl)-7,8-dihydro-1,6-naphthyridin-
6(5H)-yl)-4H-pyrimido[1,2-b]pyridazin-4-one
2,8,9-trimethyl-7-(3-(quinolin-3-yl)-7,8-dihydro-1,6-naphthyridin-6(5H)-yl)-4H-
pyrimido[1,2-b]pyridazin-4-one
7-(3-(6-(dimethylamino)pyridin-3-yl)-7,8-dihydro-1,6-naphthyridin-6(5H)-yl)-2,8,9-
trimethyl-4H-pyrimido[1,2-b]pyridazin-4-one
2,8,9-trimethyl-7-(3-(2-methylpyridin-3-yl)-7,8-dihydro-1,6-naphthyridin-6(5H)-yl)-4H-
pyrimido[1,2-b]pyridazin-4-one
7-(3-(5-chloropyridin-3-yl)-7,8-dihydro-1,6-naphthyridin-6(5H)-yl)-2,8,9-trimethyl-4H-
pyrimido[1,2-b]pyridazin-4-one
2,8,9-trimethyl-7-(3-(5-(trifluoromethyl)pyridin-3-yl)-7,8-dihydro-1,6-naphthyridin-6(5H)-
yl)-4H-pyrimido[1,2-b]pyridazin-4-one
2,8,9-trimethyl-7-(3-(pyrazin-2-yl)-7,8-dihydro-1,6-naphthyridin-6(5H)-yl)-4H-
pyrimido[1,2-b]pyridazin-4-one
7-(3-(4-chloropyridin-3-yl)-7,8-dihydro-1,6-naphthyridin-6(5H)-yl)-2,8,9-trimethyl-4H-
pyrimido[1,2-b]pyridazin-4-one
2,8,9-trimethyl-7-(3-(5-methylpyridin-3-yl)-7,8-dihydro-1,6-naphthyridin-6(5H)-yl)-4H-
pyrimido[1,2-b]pyridazin-4-one
7-(3-(6-cyclopropylpyridin-3-yl)-7,8-dihydro-1,6-naphthyridin-6(5H)-yl)-2,8,9-trimethyl-
4H-pyrimido[1,2-b]pyridazin-4-one
2,8,9-trimethyl-7-(3-(pyridin-3-yl)-7,8-dihydro-1,6-naphthyridin-6(5H)-yl)-4H-
pyrimido[1,2-b]pyridazin-4-one
2,8,9-trimethyl-7-(3-(6-(trifluoromethyl)pyridin-3-yl)-7,8-dihydro-1,6-naphthyridin-6(5H)-
yl)-4H-pyrimido[1,2-b]pyridazin-4-one
2,8,9-trimethyl-7-(3-(7-methylimidazo[1,2-a]pyridin-6-yl)-7,8-dihydro-1,6-naphthyridin-
6(5H)-yl)-4H-pyrimido[1,2-b]pyridazin-4-one
2,8,9-trimethyl-7-(3-(pyridazin-4-yl)-7,8-dihydro-1,6-naphthyridin-6(5H)-yl)-4H-
pyrimido[1,2-b]pyridazin-4-one
7-(3-(imidazo[1,2-a]pyridin-6-yl)-7,8-dihydro-1,6-naphthyridin-6(5H)-yl)-2,8,9-trimethyl-
4H-pyrimido[1,2-b]pyridazin-4-one
7-(3-(5-fluoropyridin-3-yl)-7,8-dihydro-1,6-naphthyridin-6(5H)-yl)-2,8,9-trimethyl-4H-
pyrimido[1,2-b]pyridazin-4-one
2,8,9-trimethyl-7-(3-(p-tolyl)-7,8-dihydro-1,6-naphthyridin-6(5H)-yl)-4H-pyrimido[1,2-
b]pyridazin-4-one
7-(3-(2,4-dimethylphenyl)-7,8-dihydro-1,6-naphthyridin-6(5H)-yl)-2,8,9-trimethyl-4H-
pyrimido[1,2-b]pyridazin-4-one
2,8,9-trimethyl-7-(3-(o-tolyl)-7,8-dihydro-1,6-naphthyridin-6(5H)-yl)-4H-pyrimido[1,2-
b]pyridazin-4-one
7-(3-(5-fluoro-2-methylphenyl)-7,8-dihydro-1,6-naphthyridin-6(5H)-yl)-2,8,9-trimethyl-
4H-pyrimido[1,2-b]pyridazin-4-one
7-(3-(6-methoxy-4-methylpyridin-3-yl)-7,8-dihydro-1,6-naphthyridin-6(5H)-yl)-8,9-
dimethyl-4H-pyrimido[1,2-b]pyridazin-4-one
7-(3-(4-methoxypyridin-3-yl)-7,8-dihydro-1,6-naphthyridin-6(5H)-yl)-8,9-dimethyl-4H-
pyrimido[1,2-b]pyridazin-4-one
8,9-dimethyl-7-(3-(pyridazin-4-yl)-7,8-dihydro-1,6-naphthyridin-6(5H)-yl)-4H-
pyrimido[1,2-b]pyridazin-4-one
8,9-dimethyl-7-(3-(4-(trifluoromethyl)pyridin-3-yl)-7,8-dihydro-1,6-naphthyridin-6(5H)-
yl)-4H-pyrimido[1,2-b]pyridazin-4-one
7-(3-(3-fluoro-4-methylphenyl)-7,8-dihydro-1,6-naphthyridin-6(5H)-yl)-8,9-dimethyl-4H-
pyrimido[1,2-b]pyridazin-4-one
7-(3-(5-chloropyridin-3-yl)-7,8-dihydro-1,6-naphthyridin-6(5H)-yl)-8,9-dimethyl-4H-
pyrimido[1,2-b]pyridazin-4-one
7-(3-(4-chloropyridin-3-yl)-7,8-dihydro-1,6-naphthyridin-6(5H)-yl)-8,9-dimethyl-4H-
pyrimido[1,2-b]pyridazin-4-one
7-(3-(6-chloro-5-fluoropyridin-3-yl)-7,8-dihydro-1,6-naphthyridin-6(5)-yl)-8,9-dimethyl-
4H-pyrimido[1,2-b]pyridazin-4-one
8,9-dimethyl-7-(3-(5-methylpyridin-3-yl)-7,8-dihydro-1,6-naphthyridin-6(5H)-yl)-4H-
pyrimido[1,2-b]pyridazin-4-one
8,9-dimethyl-7-(3-(pyridin-3-yl)-7,8-dihydro-1,6-naphthyridin-6(5H)-yl)-4H-pyrimido[1,2-
b]pyridazin-4-one
7-(3-(6-fluoro-4-methylpyridin-3-yl)-7,8-dihydro-1,6-naphthyridin-6(5H)-yl)-8,9-dimethyl-
4H-pyrimido[1,2-b]pyridazin-4-one
8,9-dimethyl-7-(3-(1-(tetrahydro-2H-pyran-2-yl)-1H-pyrazol-4-yl)-7,8-dihydro-1,6-
naphthyridin-6(5H)-yl)-4H-pyrimido[1,2-b]pyridazin-4-one
8,9-dimethyl-7-(3-(1-(oxetan-3-yl)-1H-pyrazol-5-yl)-7,8-dihydro-1,6-naphthyridin-6(5H)-
yl)-4H-pyrimido[1,2-b]pyridazin-4-one
7-(3-(1-cyclopropyl-1H-pyrazol-4-yl)-7,8-dihydro-1,6-naphthyridin-6(5H)-yl)-8,9-
dimethyl-4H-pyrimido[1,2-b]pyridazin-4-one
8,9-dimethyl-7-(3-(1-(pyridin-2-yl)-1H-pyrazol-4-yl)-7,8-dihydro-1,6-naphthyridin-6(5H)-
yl)-4H-pyrimido[1,2-b]pyridazin-4-one
2-(difluoromethyl)-8,9-dimethyl-7-(3-(5-(trifluoromethyl)pyridin-3-yl)-7,8-dihydro-1,6-
naphthyridin-6(5H)-yl)-4H-pyrimido[1,2-b]pyridazin-4-one
2-(difluoromethyl)-8,9-dimethyl-7-(3-(pyridin-3-yl)-7,8-dihydro-1,6-naphthyridin-6(5H)-
yl)-4H-pyrimido[1,2-b]pyridazin-4-one
2-(difluoromethyl)-7-(3-(5-fluoropyridin-3-yl)-7,8-dihydro-1,6-naphthyridin-6(5H)-yl)-8,9-
dimethyl-4H-pyrimido[1,2-b]pyridazin-4-one
5-(6-(2-(difluoromethyl)-8,9-dimethyl-4-oxo-4H-pyrimido[1,2-b]pyridazin-7-yl)-5,6,7,8-
tetrahydro-1,6-naphthyridin-3-yl)nicotinonitrile
2-(difluoromethyl)-8,9-dimethyl-7-(3-(pyridazin-4-yl)-7,8-dihydro-1,6-naphthyridin-6(5H)-
yl)-4H-pyrimido[1,2-b]pyridazin-4-one
2-(difluoromethyl)-8,9-dimethyl-7-(3-(pyrimidin-5-yl)-7,8-dihydro-1,6-naphthyridin-6(5H)-
yl)-4H-pyrimido[1,2-b]pyridazin-4-one
7-(3-(5-chloropyridin-3-yl)-7,8-dihydro-1,6-naphthyridin-6(5H)-yl)-2-(difluoromethyl)-8,9-
dimethyl-4H-pyrimido[1,2-b]pyridazin-4-one
2-(difluoromethyl)-8,9-dimethyl-7-(3-(4-methylpyridin-3-yl)-7,8-dihydro-1,6-naphthyridin-
6(5H)-yl)-4H-pyrimido[1,2-b]pyridazin-4-one
2-(difluoromethyl)-8,9-dimethyl-7-(3-(7-methylimidazo[1,2-a]pyridin-6-yl)-7,8-dihydro-
1,6-naphthyridin-6(5H)-yl)-4H-pyrimido[1,2-b]pyridazin-4-one
2-(difluoromethyl)-8,9-dimethyl-7-(3-(1-(pyridin-2-yl)-1H-pyrazol-4-yl)-7,8-dihydro-1,6-
naphthyridin-6(5H)-yl)-4H-pyrimido[1,2-b]pyridazin-4-one
2-(difluoromethyl)-7-(3-(2,4-dimethylphenyl)-7,8-dihydro-1,6-naphthyridin-6(5H)-yl)-8,9-
dimethyl-4H-pyrimido[1,2-b]pyridazin-4-one
2-(difluoromethyl)-7-(3-(1-(2-fluorophenyl)-1H-pyrazol-4-yl)-7,8-dihydro-1,6-
naphthyridin-6(5H)-yl)-8,9-dimethyl-4H-pyrimido[1,2-b]pyridazin-4-one
2-(difluoromethyl)-7-(3-(1-ethyl-1/-pyrazol-4-yl)-7,8-dihydro-1,6-naphthyridin-6(5H)-yl)-
8,9-dimethyl-4H-pyrimido[1,2-b]pyridazin-4-one
2-(difluoromethyl)-7-(3-(5-methoxypyridin-3-yl)-7,8-dihydro-1,6-naphthyridin-6(5)-yl)-
8,9-dimethyl-4H-pyrimido[1,2-b]pyridazin-4-one
7-(3-(5-chloro-6-methoxypyridin-3-yl)-7,8-dihydro-1,6-naphthyridin-6(5H)-yl)-2-
(difluoromethyl)-8,9-dimethyl-4H-pyrimido[1,2-b]pyridazin-4-one
2-(difluoromethyl)-8,9-dimethyl-7-(3-(p-tolyl)-7,8-dihydro-1,6-naphthyridin-6(5H)-yl)-4H-
pyrimido[1,2-b]pyridazin-4-one
2-(difluoromethyl)-7-(3-(6-methoxypyridin-3-yl)-7,8-dihydro-1,6-naphthyridin-6(5H)-yl)-
8,9-dimethyl-4H-pyrimido[1,2-b]pyridazin-4-one
2-(difluoromethyl)-7-(3-(5-fluoro-2-methylphenyl)-7,8-dihydro-1,6-naphthyridin-6(5H)-
yl)-8,9-dimethyl-4H-pyrimido[1,2-b]pyridazin-4-one
2-(difluoromethyl)-7-(3-(1-(difluoromethyl)-1H-pyrazol-4-yl)-7,8-dihydro-1,6-
naphthyridin-6(5H)-yl)-8,9-dimethyl-4H-pyrimido[1,2-b]pyridazin-4-one
2-(difluoromethyl)-8,9-dimethyl-7-(3-(1-(2-morpholinoethyl)-1H-pyrazol-4-yl)-7,8-
dihydro-1,6-naphthyridin-6(5H)-yl)-4H-pyrimido[1,2-b]pyridazin-4-one
7-(3-(2,6-dichloropyridin-3-yl)-7,8-dihydro-1,6-naphthyridin-6(5H)-yl)-2-(difluoromethyl)-
8,9-dimethyl-4H-pyrimido[1,2-b]pyridazin-4-one
2,8,9-trimethyl-7-(3-(1-(pyridin-2-yl)-1/-pyrazol-4-yl)-7,8-dihydro-1,6-naphthyridin-
6(5H)-yl)-4H-pyrimido[1,2-b]pyridazin-4-one
2,8,9-trimethyl-7-(3-(1-(oxetan-3-yl)-1H-pyrazol-5-yl)-7,8-dihydro-1,6-naphthyridin-
6(5H)-yl)-4H-pyrimido[1,2-b]pyridazin-4-one
7-(3-(1-(2-(dimethylamino)ethyl)-1H-pyrazol-4-yl)-7,8-dihydro-1,6-naphthyridin-6(5H)-
yl)-2,8,9-trimethyl-4H-pyrimido[1,2-b]pyridazin-4-one
2,8,9-trimethyl-7-(3-(1-(2-morpholinoethyl)-1H-pyrazol-4-yl)-7,8-dihydro-1,6-
naphthyridin-6(5H)-yl)-4H-pyrimido[1,2-b]pyridazin-4-one
2,8,9-trimethyl-7-(3-(1-(oxetan-3-yl)-1H-pyrazol-4-yl)-7,8-dihydro-1,6-naphthyridin-
6(5H)-yl)-4H-pyrimido[1,2-b]pyridazin-4-one
7-(3-(1-(2-fluorophenyl)-1H-pyrazol-4-yl)-7,8-dihydro-1,6-naphthyridin-6(5H)-yl)-2,8,9-
trimethyl-4H-pyrimido[1,2-b]pyridazin-4-one
2,8,9-trimethyl-7-(3-(pyrazolo[1,5-a]pyridin-3-yl)-7,8-dihydro-1,6-naphthyridin-6(5H)-yl)-
4H-pyrimido[1,2-b]pyridazin-4-one
2,8,9-trimethyl-7-(3-(1-(tetrahydro-2H-pyran-4-yl)-1H-pyrazol-4-yl)-7,8-dihydro-1,6-
naphthyridin-6(5H)-yl)-4H-pyrimido[1,2-b]pyridazin-4-one
7-(3-(1-cyclopropyl-1H-pyrazol-4-yl)-7,8-dihydro-1,6-naphthyridin-6(5H)-yl)-2,8,9-
trimethyl-4H-pyrimido[1,2-b]pyridazin-4-one
2,8,9-trimethyl-7-(3-(1-(2,2,2-trifluoroethyl)-1H-pyrazol-4-yl)-7,8-dihydro-1,6-
naphthyridin-6(5H)-yl)-4H-pyrimido[1,2-b]pyridazin-4-one
7-(3-(1,5-dimethyl-1H-pyrazol-4-yl)-7,8-dihydro-1,6-naphthyridin-6(5H)-yl)-2,8,9-
trimethyl-4H-pyrimido[1,2-b]pyridazin-4-one
7-(3-(6-methoxypyridin-3-yl)-7,8-dihydro-1,6-naphthyridin-6(5/)-yl)-2,8,9-trimethyl-4H-
pyrimido[1,2-b]pyridazin-4-one
7-(3-(4-chloro-6-methylpyridin-3-yl)-7,8-dihydro-1,6-naphthyridin-6(5H)-yl)-2-
(difluoromethyl)-8,9-dimethyl-4H-pyrimido[1,2-b]pyridazin-4-one
2-(difluoromethyl)-7-(3-(6-methoxy-4-methylpyridin-3-yl)-7,8-dihydro-1,6-naphthyridin-
6(5H)-yl)-8,9-dimethyl-4H-pyrimido[1,2-b]pyridazin-4-one
2-(difluoromethyl)-8,9-dimethyl-7-(3-(4-(trifluoromethyl)pyridin-3-yl)-7,8-dihydro-1,6-
naphthyridin-6(5H)-yl)-4H-pyrimido[1,2-b]pyridazin-4-one
2-(difluoromethyl)-7-(3-(4-methoxypyridin-3-yl)-7,8-dihydro-1,6-naphthyridin-6(5H)-yl)-
8,9-dimethyl-4H-pyrimido[1,2-b]pyridazin-4-one
2-(difluoromethyl)-7-(3-(3-fluoro-4-methylphenyl)-7,8-dihydro-1,6-naphthyridin-6(5H)-
yl)-8,9-dimethyl-4H-pyrimido[1,2-b]pyridazin-4-one
8,9-dimethyl-7-(3-(1-(tetrahydro-2H-pyran-4-yl)-1/-pyrazol-4-yl)-7,8-dihydro-1,6-
naphthyridin-6(5H)-yl)-4H-pyrimido[1,2-b]pyridazin-4-one
7-(3-(1-(2-fluorophenyl)-1H-pyrazol-4-yl)-7,8-dihydro-1,6-naphthyridin-6(5H)-yl)-8,9-
dimethyl-4H-pyrimido[1,2-b]pyridazin-4-one
7-(3-(5-fluoro-2-methylphenyl)-7,8-dihydro-1,6-naphthyridin-6(5H)-yl)-8,9-dimethyl-4H-
pyrimido[1,2-b]pyridazin-4-one
2-(2-hydroxypropan-2-yl)-8-methyl-7-(3-(trifluoromethyl)-7,8-dihydro-1,6-naphthyridin-
6(5H)-yl)-4H-pyrimido[1,2-b]pyridazin-4-one
7-(3-(3-fluoro-4-methylphenyl)-7,8-dihydro-1,6-naphthyridin-6(5H)-yl)-2,8,9-trimethyl-
4H-pyrimido[1,2-b]pyridazin-4-one
7-(3-(6-chloro-4-methylpyridin-3-yl)-7,8-dihydro-1,6-naphthyridin-6(5H)-yl)-2,8,9-
trimethyl-4H-pyrimido[1,2-b]pyridazin-4-one
7-(3-(6-methoxy-4-methylpyridin-3-yl)-7,8-dihydro-1,6-naphthyridin-6(5H)-yl)-2,8,9-
trimethyl-4H-pyrimido[1,2-b]pyridazin-4-one
7-(3-(5-methoxypyridin-3-yl)-7,8-dihydro-1,6-naphthyridin-6(5H)-yl)-2,8,9-trimethyl-4H-
pyrimido[1,2-b]pyridazin-4-one
2,8,9-trimethyl-7-(3-(2,4,5-trifluorophenyl)-7,8-dihydro-1,6-naphthyridin-6(5H)-yl)-4H-
pyrimido[1,2-b]pyridazin-4-one
7-(3-(3-fluoro-4-methoxyphenyl)-7,8-dihydro-1,6-naphthyridin-6(5H)-yl)-2,8,9-trimethyl-
4H-pyrimido[1,2-b]pyridazin-4-one
7-(3-(6-fluoro-4-methylpyridin-3-yl)-7,8-dihydro-1,6-naphthyridin-6(5H)-yl)-2,8,9-
trimethyl-4H-pyrimido[1,2-b]pyridazin-4-one
7-(3-(3-fluorophenyl)-7,8-dihydro-1,6-naphthyridin-6(5H)-yl)-2,8,9-trimethyl-4H-
pyrimido[1,2-b]pyridazin-4-one
7-(3-(6-fluoropyridin-3-yl)-7,8-dihydro-1,6-naphthyridin-6(5H)-yl)-2,8,9-trimethyl-4H-
pyrimido[1,2-b]pyridazin-4-one
7-(3-(3,4-difluorophenyl)-7,8-dihydro-1,6-naphthyridin-6(5H)-yl)-2,8,9-trimethyl-4H-
pyrimido[1,2-b]pyridazin-4-one
7-(3-(3-fluoro-4-methylphenyl)-7,8-dihydro-1,6-naphthyridin-6(5H)-yl)-2,8-dimethyl-4H-
pyrimido[1,2-b]pyridazin-4-one
7-(3-(6-chloro-4-methylpyridin-3-yl)-7,8-dihydro-1,6-naphthyridin-6(5H)-yl)-2,8-dimethyl-
4H-pyrimido[1,2-b]pyridazin-4-one
2,8-dimethyl-7-(3-(2,4,5-trifluorophenyl)-7,8-dihydro-1,6-naphthyridin-6(5H)-yl)-4H-
pyrimido[1,2-b]pyridazin-4-one
7-(3-(6-fluoro-4-methylpyridin-3-yl)-7,8-dihydro-1,6-naphthyridin-6(5H)-yl)-2,8-dimethyl-
4H-pyrimido[1,2-b]pyridazin-4-one
7-(3-(5-fluoro-2-methylphenyl)-7,8-dihydro-1,6-naphthyridin-6(5/)-yl)-2,8-dimethyl-4H-
pyrimido[1,2-b]pyridazin-4-one
7-(3-(3-fluorophenyl)-7,8-dihydro-1,6-naphthyridin-6(5H)-yl)-2,8-dimethyl-4H-
pyrimido[1,2-b]pyridazin-4-one
7-(3-(6-fluoropyridin-3-yl)-7,8-dihydro-1,6-naphthyridin-6(5/)-yl)-2,8-dimethyl-4H-
pyrimido[1,2-b]pyridazin-4-one
7-(3-(3,4-difluorophenyl)-7,8-dihydro-1,6-naphthyridin-6(5H)-yl)-2,8-dimethyl-4H-
pyrimido[1,2-b]pyridazin-4-one
2,8-dimethyl-7-(3-(4-methylpyridin-3-yl)-7,8-dihydro-1,6-naphthyridin-6(5H)-yl)-4H-
pyrimido[1,2-b]pyridazin-4-one
7-(3-(1-(difluoromethyl)-1H-pyrazol-4-yl)-7,8-dihydro-1,6-naphthyridin-6(5H)-yl)-2,8-
dimethyl-4H-pyrimido[1,2-b]pyridazin-4-one
7-(3-(3-fluoro-4-methoxyphenyl)-7,8-dihydro-1,6-naphthyridin-6(5H)-yl)-2,8-dimethyl-4H-
pyrimido[1,2-b]pyridazin-4-one
2,8-dimethyl-7-(3-(7-methylimidazo[1,2-a]pyridin-6-yl)-7,8-dihydro-1,6-naphthyridin-
6(5H)-yl)-4H-pyrimido[1,2-b]pyridazin-4-one
2,8-dimethyl-7-(3-(1,3,5-trimethyl-1H-pyrazol-4-yl)-7,8-dihydro-1,6-naphthyridin-6(5H)-
yl)-4H-pyrimido[1,2-b]pyridazin-4-one
7-(3-(1-ethyl-1H-pyrazol-4-yl)-7,8-dihydro-1,6-naphthyridin-6(5)-yl)-2,8-dimethyl-4/-
pyrimido[1,2-b]pyridazin-4-one
7-(3-(imidazo[1,2-a]pyridin-6-yl)-7,8-dihydro-1,6-naphthyridin-6(5H)-yl)-2,8-dimethyl-
4H-pyrimido[1,2-b]pyridazin-4-one
7-(3-(6-(dimethylamino)pyridin-3-yl)-7,8-dihydro-1,6-naphthyridin-6(5H)-yl)-2,8-
dimethyl-4H-pyrimido[1,2-b]pyridazin-4-one
8,9-dimethyl-7-(3-(o-tolyl)-7,8-dihydro-1,6-naphthyridin-6(5H)-yl)-4H-pyrimido[1,2-
b]pyridazin-4-one
5-(6-(8,9-dimethyl-4-oxo-4H-pyrimido[1,2-b]pyridazin-7-yl)-5,6,7,8-tetrahydro-1,6-
naphthyridin-3-yl)nicotinonitrile
8,9-dimethyl-7-(3-(quinolin-3-yl)-7,8-dihydro-1,6-naphthyridin-6(5H)-yl)-4H-
pyrimido[1,2-b]pyridazin-4-one
7-(3-(imidazo[1,2-a]pyridin-6-yl)-7,8-dihydro-1,6-naphthyridin-6(5H)-yl)-8,9-dimethyl-
4H-pyrimido[1,2-b]pyridazin-4-one
2-(cyclopentylmethyl)-8,9-dimethyl-7-(3-(6-methylpyridin-3-yl)-7,8-dihydro-1,6-
naphthyridin-6(5H)-yl)-4H-pyrimido[1,2-b]pyridazin-4-one
7-(3-(5-fluoro-2-methylphenyl)-7,8-dihydro-1,6-naphthyridin-6(5H)-yl)-2-
(methoxymethyl)-8,9-dimethyl-4H-pyrimido[1,2-b]pyridazin-4-one
2-(methoxymethyl)-8,9-dimethyl-7-(3-(4-methylpyridin-3-yl)-7,8-dihydro-1,6-
naphthyridin-6(5H)-yl)-4H-pyrimido[1,2-b]pyridazin-4-one
7-(3-(6-fluoro-4-methylpyridin-3-yl)-7,8-dihydro-1,6-naphthyridin-6(5H)-yl)-2-
(methoxymethyl)-8,9-dimethyl-4H-pyrimido[1,2-b]pyridazin-4-one
7-(3-(2-isopropylphenyl)-7,8-dihydro-1,6-naphthyridin-6(5H)-yl)-2,8-dimethyl-4H-
pyrimido[1,2-b]pyridazin-4-one
7-(3-(2-cyclopropylphenyl)-7,8-dihydro-1,6-naphthyridin-6(5H)-yl)-2,8-dimethyl-4H-
pyrimido[1,2-b]pyridazin-4-one
7-(3-(2-ethylphenyl)-7,8-dihydro-1,6-naphthyridin-6(5H)-yl)-2,8-dimethyl-4H-
pyrimido[1,2-b]pyridazin-4-one
7-(3-(2-methoxyphenyl)-7,8-dihydro-1,6-naphthyridin-6(5)-yl)-2,8-dimethyl-4H-
pyrimido[1,2-b]pyridazin-4-one
7-(3-(2-fluorophenyl)-7,8-dihydro-1,6-naphthyridin-6(5H)-yl)-2,8-dimethyl-4H-
pyrimido[1,2-b]pyridazin-4-one
7-(3-(2-hydroxyphenyl)-7,8-dihydro-1,6-naphthyridin-6(5H)-yl)-2,8-dimethyl-4H-
pyrimido[1,2-b]pyridazin-4-one
7-(3-(2,5-difluorophenyl)-7,8-dihydro-1,6-naphthyridin-6(5H)-yl)-2,8-dimethyl-4H-
pyrimido[1,2-b]pyridazin-4-one
2,8-dimethyl-7-(3-(o-tolyl)-7,8-dihydro-1,6-naphthyridin-6(5H)-yl)-4H-pyrimido[1,2-
b]pyridazin-4-one
7-(3-(2-fluoro-5-methylpyridin-4-yl)-7,8-dihydro-1,6-naphthyridin-6(5H)-yl)-2,8-dimethyl-
4H-pyrimido[1,2-b]pyridazin-4-one
2,8-dimethyl-7-(3-(2-methylpyridin-4-yl)-7,8-dihydro-1,6-naphthyridin-6(5H)-yl)-4H-
pyrimido[1,2-b]pyridazin-4-one
2,8-dimethyl-7-(3-(pyridin-4-yl)-7,8-dihydro-1,6-naphthyridin-6(5H)-yl)-4H-pyrimido[1,2-
b]pyridazin-4-one
2,8-dimethyl-7-(3-(3-methylpyridin-4-yl)-7,8-dihydro-1,6-naphthyridin-6(5H)-yl)-4H-
pyrimido[1,2-b]pyridazin-4-one
7-(3-(2,6-difluoropyridin-4-yl)-7,8-dihydro-1,6-naphthyridin-6(5H)-yl)-2,8-dimethyl-4H-
pyrimido[1,2-b]pyridazin-4-one
2,8,9-trimethyl-7-(3-(2-methylpyridin-4-yl)-7,8-dihydro-1,6-naphthyridin-6(5H)-yl)-4H-
pyrimido[1,2-b]pyridazin-4-one
2,8,9-trimethyl-7-(3-(pyridin-4-yl)-7,8-dihydro-1,6-naphthyridin-6(5H)-yl)-4H-
pyrimido[1,2-b]pyridazin-4-one
2,8,9-trimethyl-7-(3-(3-methylpyridin-4-yl)-7,8-dihydro-1,6-naphthyridin-6(5H)-yl)-4H-
pyrimido[1,2-b]pyridazin-4-one
7-(3-(2,6-difluoropyridin-4-yl)-7,8-dihydro-1,6-naphthyridin-6(5H)-yl)-2,8,9-trimethyl-4H-
pyrimido[1,2-b]pyridazin-4-one
7-(3-(2-isopropylphenyl)-7,8-dihydro-1,6-naphthyridin-6(5H)-yl)-2,8,9-trimethyl-4H-
pyrimido[1,2-b]pyridazin-4-one
7-(3-(2-cyclopropylphenyl)-7,8-dihydro-1,6-naphthyridin-6(5H)-yl)-2,8,9-trimethyl-4H-
pyrimido[1,2-b]pyridazin-4-one
7-(3-(2-ethylphenyl)-7,8-dihydro-1,6-naphthyridin-6(5H)-yl)-2,8,9-trimethyl-4H-
pyrimido[1,2-b]pyridazin-4-one
7-(3-(2-methoxyphenyl)-7,8-dihydro-1,6-naphthyridin-6(5H)-yl)-2,8,9-trimethyl-4H-
pyrimido[1,2-b]pyridazin-4-one
7-(3-(2-fluorophenyl)-7,8-dihydro-1,6-naphthyridin-6(5/)-yl)-2,8,9-trimethyl-4H-
pyrimido[1,2-b]pyridazin-4-one
7-(3-(2,3-difluoro-5-methylphenyl)-7,8-dihydro-1,6-naphthyridin-6(5H)-yl)-2,8-dimethyl-
4H-pyrimido[1,2-b]pyridazin-4-one
2,8-dimethyl-7-(3-(2,3,5-trifluorophenyl)-7,8-dihydro-1,6-naphthyridin-6(5H)-yl)-4H-
pyrimido[1,2-b]pyridazin-4-one
2,8-dimethyl-7-(3-(2-(trifluoromethoxy)phenyl)-7,8-dihydro-1,6-naphthyridin-6(5H)-yl)-
4H-pyrimido[1,2-b]pyridazin-4-one
2,8-dimethyl-7-(3-(2-(trifluoromethyl)phenyl)-7,8-dihydro-1,6-naphthyridin-6(5H)-yl)-4H-
pyrimido[1,2-b]pyridazin-4-one
7-(3-(2-chlorophenyl)-7,8-dihydro-1,6-naphthyridin-6(5H)-yl)-2,8-dimethyl-4H-
pyrimido[1,2-b]pyridazin-4-one
7-(3-(2,4-difluorophenyl)-7,8-dihydro-1,6-naphthyridin-6(5H)-yl)-2,8-dimethyl-4H-
pyrimido[1,2-b]pyridazin-4-one
7-(3-(2,5-difluoropyridin-4-yl)-7,8-dihydro-1,6-naphthyridin-6(5H)-yl)-2,8-dimethyl-4H-
pyrimido[1,2-b]pyridazin-4-one
7-(3-(3-methoxypyridin-4-yl)-7,8-dihydro-1,6-naphthyridin-6(5H)-yl)-2,8-dimethyl-4H-
pyrimido[1,2-b]pyridazin-4-one
2,8-dimethyl-7-(3-(pyrazin-2-yl)-7,8-dihydro-1,6-naphthyridin-6(5H)-yl)-4H-pyrimido[1,2-
b]pyridazin-4-one
2-(methoxymethyl)-8,9-dimethyl-7-(3-(pyrazin-2-yl)-7,8-dihydro-1,6-naphthyridin-6(5H)-
yl)-4H-pyrimido[1,2-b]pyridazin-4-one
7-(3-(6-methoxy-4-methylpyridin-3-yl)-7,8-dihydro-1,6-naphthyridin-6(5H)-yl)-2-
(methoxymethyl)-8,9-dimethyl-4H-pyrimido[1,2-b]pyridazin-4-one
7-(3-(1-cyclopropyl-1H-pyrazol-4-yl)-7,8-dihydro-1,6-naphthyridin-6(5H)-yl)-2-
(methoxymethyl)-8,9-dimethyl-4H-pyrimido[1,2-b]pyridazin-4-one
7-(3-(4-fluoro-2-methylphenyl)-7,8-dihydro-1,6-naphthyridin-6(5H)-yl)-8,9-dimethyl-4H-
pyrimido[1,2-b]pyridazin-4-one
8,9-dimethyl-7-(3-(2-(trifluoromethoxy)phenyl)-7,8-dihydro-1,6-naphthyridin-6(5H)-yl)-
4H-pyrimido[1,2-b]pyridazin-4-one
8,9-dimethyl-7-(3-(2-(trifluoromethyl)phenyl)-7,8-dihydro-1,6-naphthyridin-6(5H)-yl)-4H-
pyrimido[1,2-b]pyridazin-4-one
7-(3-(2-chlorophenyl)-7,8-dihydro-1,6-naphthyridin-6(5H)-yl)-8,9-dimethyl-4H-
pyrimido[1,2-b]pyridazin-4-one
7-(3-(2,5-difluoropyridin-4-yl)-7,8-dihydro-1,6-naphthyridin-6(5H)-yl)-8,9-dimethyl-4H-
pyrimido[1,2-b]pyridazin-4-one
7-(3-(3-methoxypyridin-4-yl)-7,8-dihydro-1,6-naphthyridin-6(5)-yl)-8,9-dimethyl-4H-
pyrimido[1,2-b]pyridazin-4-one
8-(methoxymethyl)-9-methyl-7-(3-(trifluoromethyl)-7,8-dihydro-1,6-naphthyridin-6(5H)-
yl)-4H-pyrimido[1,2-b]pyridazin-4-one
8-(methoxymethyl)-9-methyl-7-(3-(6-methylpyridin-3-yl)-7,8-dihydro-1,6-naphthyridin-
6(5H)-yl)-4H-pyrimido[1,2-b]pyridazin-4-one
7-(3-(2,3-difluoro-5-methylphenyl)-7,8-dihydro-1,6-naphthyridin-6(5H)-yl)-2,8,9-
trimethyl-4H-pyrimido[1,2-b]pyridazin-4-one
2,8,9-trimethyl-7-(3-(2,3,5-trifluorophenyl)-7,8-dihydro-1,6-naphthyridin-6(5H)-yl)-4H-
pyrimido[1,2-b]pyridazin-4-one
7-(3-(4-fluoro-2-methylphenyl)-7,8-dihydro-1,6-naphthyridin-6(5H)-yl)-2,8,9-trimethyl-
4H-pyrimido[1,2-b]pyridazin-4-one
2,8,9-trimethyl-7-(3-(2-(trifluoromethoxy)phenyl)-7,8-dihydro-1,6-naphthyridin-6(5H)-yl)-
4H-pyrimido[1,2-b]pyridazin-4-one
9-(methoxymethyl)-8-methyl-7-(3-(trifluoromethyl)-7,8-dihydro-1,6-naphthyridin-6(5H)-
yl)-4H-pyrimido[1,2-b]pyridazin-4-one
2,8,9-trimethyl-7-(3-(2-(trifluoromethyl)phenyl)-7,8-dihydro-1,6-naphthyridin-6(5H)-yl)-
4H-pyrimido[1,2-b]pyridazin-4-one
7-(3-(2-chlorophenyl)-7,8-dihydro-1,6-naphthyridin-6(5H)-yl)-2,8,9-trimethyl-4H-
pyrimido[1,2-b]pyridazin-4-one
7-(3-(2,4-difluorophenyl)-7,8-dihydro-1,6-naphthyridin-6(5H)-yl)-2,8,9-trimethyl-4H-
pyrimido[1,2-b]pyridazin-4-one
7-(3-(2-isopropoxyphenyl)-7,8-dihydro-1,6-naphthyridin-6(5H)-yl)-2,8,9-trimethyl-4H-
pyrimido[1,2-b]pyridazin-4-one
7-(3-(2,5-difluoropyridin-4-yl)-7,8-dihydro-1,6-naphthyridin-6(5H)-yl)-2,8,9-trimethyl-4H-
pyrimido[1,2-b]pyridazin-4-one
7-(3-(3-methoxypyridin-4-yl)-7,8-dihydro-1,6-naphthyridin-6(5/)-yl)-2,8,9-trimethyl-4H-
pyrimido[1,2-b]pyridazin-4-one
2-(difluoromethyl)-7-(3-(3,4-difluorophenyl)-7,8-dihydro-1,6-naphthyridin-6(5H)-yl)-8,9-
dimethyl-4H-pyrimido[1,2-b]pyridazin-4-one
7-(3-(2,3-difluoro-5-methylphenyl)-7,8-dihydro-1,6-naphthyridin-6(5H)-yl)-2-
(difluoromethyl)-8,9-dimethyl-4H-pyrimido[1,2-b]pyridazin-4-one
7-(3-(6-methoxy-4-methylpyridin-3-yl)-7,8-dihydro-1,6-naphthyridin-6(5H)-yl)-2,8-
dimethyl-4H-pyrimido[1,2-b]pyridazin-4-one
2,8-dimethyl-7-(3-(pyrimidin-5-yl)-7,8-dihydro-1,6-naphthyridin-6(5H)-yl)-4H-
pyrimido[1,2-b]pyridazin-4-one
2,8-dimethyl-7-(3-(1-(oxetan-3-yl)-1H-pyrazol-4-yl)-7,8-dihydro-1,6-naphthyridin-6(5H)-
yl)-4H-pyrimido[1,2-b]pyridazin-4-one
2,8-dimethyl-7-(3-(pyrazolo[1,5-a]pyridin-3-yl)-7,8-dihydro-1,6-naphthyridin-6(5H)-yl)-
4H-pyrimido[1,2-b]pyridazin-4-one
2,8-dimethyl-7-(3-(5-methylpyridin-3-yl)-7,8-dihydro-1,6-naphthyridin-6(5H)-yl)-4H-
pyrimido[1,2-b]pyridazin-4-one
2,8-dimethyl-7-(3-(pyridin-3-yl)-7,8-dihydro-1,6-naphthyridin-6(5H)-yl)-4H-pyrimido[1,2-
b]pyridazin-4-one
2,8-dimethyl-7-(3-(1-(2,2,2-trifluoroethyl)-1H-pyrazol-4-yl)-7,8-dihydro-1,6-naphthyridin-
6(5H)-yl)-4H-pyrimido[1,2-b]pyridazin-4-one
7-(3-(2,4-dimethylphenyl)-7,8-dihydro-1,6-naphthyridin-6(5H)-yl)-2,8-dimethyl-4/-
pyrimido[1,2-b]pyridazin-4-one
2,8-dimethyl-7-(3-(p-tolyl)-7,8-dihydro-1,6-naphthyridin-6(5H)-yl)-4H-pyrimido[1,2-
b]pyridazin-4-one
7-(3-(1,5-dimethyl-1H-pyrazol-4-yl)-7,8-dihydro-1,6-naphthyridin-6(5H)-yl)-2,8-dimethyl-
4H-pyrimido[1,2-b]pyridazin-4-one
7-(3-(4-methoxypyridin-3-yl)-7,8-dihydro-1,6-naphthyridin-6(5H)-yl)-2,8-dimethyl-4H-
pyrimido[1,2-b]pyridazin-4-one
8,9-dimethyl-7-(3-(7-methylimidazo[1,2-a]pyridin-6-yl)-7,8-dihydro-1,6-naphthyridin-
6(5H)-yl)-4H-pyrimido[1,2-b]pyridazin-4-one
2,8-dimethyl-7-(3-(thiophen-2-yl)-7,8-dihydro-1,6-naphthyridin-6(5H)-yl)-4H-
pyrimido[1,2-b]pyridazin-4-one
7-(3-(3-methoxythiophen-2-yl)-7,8-dihydro-1,6-naphthyridin-6(5H)-yl)-2,8-dimethyl-4H-
pyrimido[1,2-b]pyridazin-4-one
7-(3-(5-(hydroxymethyl)thiophen-2-yl)-7,8-dihydro-1,6-naphthyridin-6(5H)-yl)-2,8-
dimethyl-4H-pyrimido[1,2-b]pyridazin-4-one
2,8-dimethyl-7-(3-(thiophen-3-yl)-7,8-dihydro-1,6-naphthyridin-6(5H)-yl)-4H-
pyrimido[1,2-b]pyridazin-4-one
7-(3-(furan-2-yl)-7,8-dihydro-1,6-naphthyridin-6(5H)-yl)-2,8-dimethyl-4H-pyrimido[1,2-
b]pyridazin-4-one
2,8-dimethyl-7-(3-(5-methylfuran-2-yl)-7,8-dihydro-1,6-naphthyridin-6(5H)-yl)-4H-
pyrimido[1,2-b]pyridazin-4-one
7-(3-(3-methoxythiophen-2-yl)-7,8-dihydro-1,6-naphthyridin-6(5H)-yl)-2,8,9-trimethyl-4H-
pyrimido[1,2-b]pyridazin-4-one
2,8,9-trimethyl-7-(3-(thiophen-3-yl)-7,8-dihydro-1,6-naphthyridin-6(5H)-yl)-4H-
pyrimido[1,2-b]pyridazin-4-one
2,8,9-trimethyl-7-(3-(5-methylfuran-2-yl)-7,8-dihydro-1,6-naphthyridin-6(5H)-yl)-4H-
pyrimido[1,2-b]pyridazin-4-one
2-(difluoromethyl)-7-(3-(3-fluorophenyl)-7,8-dihydro-1,6-naphthyridin-6(5/)-yl)-8,9-
dimethyl-4H-pyrimido[1,2-b]pyridazin-4-one
2-(difluoromethyl)-8,9-dimethyl-7-(3-(3-methylpyridin-4-yl)-7,8-dihydro-1,6-naphthyridin-
6(5H)-yl)-4H-pyrimido[1,2-b]pyridazin-4-one
2-(difluoromethyl)-7-(3-(1-(2-(dimethylamino)ethyl)-1H-pyrazol-4-yl)-7,8-dihydro-1,6-
naphthyridin-6(5H)-yl)-8,9-dimethyl-4HI-pyrimido[1,2-b]pyridazin-4-one
2-(difluoromethyl)-8,9-dimethyl-7-(3-(1-(oxetan-3-yl)-1H-pyrazol-4-yl)-7,8-dihydro-1,6-
naphthyridin-6(5H)-yl)-4H-pyrimido[1,2-b]pyridazin-4-one
2-(difluoromethyl)-8,9-dimethyl-7-(3-(1,3,5-trimethyl-1H-pyrazol-4-yl)-7,8-dihydro-1,6-
naphthyridin-6(5H)-yl)-4H-pyrimido[1,2-b]pyridazin-4-one
2-(difluoromethyl)-7-(3-(3-methoxythiophen-2-yl)-7,8-dihydro-1,6-naphthyridin-6(5H)-yl)-
8,9-dimethyl-4H-pyrimido[1,2-b]pyridazin-4-one
2-(difluoromethyl)-8,9-dimethyl-7-(3-(thiophen-2-yl)-7,8-dihydro-1,6-naphthyridin-6(5H)-
yl)-4H-pyrimido[1,2-b]pyridazin-4-one
2-(methoxymethyl)-8-methyl-7-(3-(trifluoromethyl)-7,8-dihydro-1,6-naphthyridin-6(5H)-
yl)-4H-pyrimido[1,2-b]pyridazin-4-one
2-(methoxymethyl)-8-methyl-7-(3-(6-methylpyridin-3-yl)-7,8-dihydro-1,6-naphthyridin-
6(5H)-yl)-4H-pyrimido[1,2-b]pyridazin-4-one
7-(3-(6-(difluoromethoxy)pyridin-3-yl)-7,8-dihydro-1,6-naphthyridin-6(5H)-yl)-8-methyl-
4H-pyrimido[1,2-b]pyridazin-4-one
5-(6-(8-methyl-4-oxo-4H-pyrimido[1,2-b]pyridazin-7-yl)-5,6,7,8-tetrahydro-1,6-
naphthyridin-3-yl)picolinonitrile
7-(3-(6-(difluoromethoxy)-4-methylpyridin-3-yl)-7,8-dihydro-1,6-naphthyridin-6(5H)-yl)-
8-methyl-4H-pyrimido[1,2-b]pyridazin-4-one
7-(3-(2,6-difluoropyridin-3-yl)-7,8-dihydro-1,6-naphthyridin-6(5H)-yl)-8-methyl-4H-
pyrimido[1,2-b]pyridazin-4-one
3-(6-(8-methyl-4-oxo-4H-pyrimido[1,2-b]pyridazin-7-yl)-5,6,7,8-tetrahydro-1,6-
naphthyridin-3-yl)isonicotinonitrile
7-(3-(2,5-difluoropyridin-4-yl)-7,8-dihydro-1,6-naphthyridin-6(5H)-yl)-2-(methoxymethyl)-
8-methyl-4H-pyrimido[1,2-b]pyridazin-4-one
8-methyl-7-(3-(1-methyl-1H-1,2,3-triazol-4-yl)-7,8-dihydro-1,6-naphthyridin-6(5H)-yl)-
4H-pyrimido[1,2-b]pyridazin-4-one
7-(3-(6-fluoropyridin-3-yl)-7,8-dihydro-1,6-naphthyridin-6(5H)-yl)-2-(methoxymethyl)-8-
methyl-4H-pyrimido[1,2-b]pyridazin-4-one
7-(3-(1,2-dimethyl-1H-imidazol-5-yl)-7,8-dihydro-1,6-naphthyridin-6(5H)-yl)-8-methyl-
4H-pyrimido[1,2-b]pyridazin-4-one
2-(methoxymethyl)-8-methyl-7-(3-(pyrimidin-5-yl)-7,8-dihydro-1,6-naphthyridin-6(5H)-
yl)-4H-pyrimido[1,2-b]pyridazin-4-one
7-(3-(6,7-dihydro-4H-pyrazolo[5,1-c][1,4]oxazin-3-yl)-7,8-dihydro-1,6-naphthyridin-
6(5H)-yl)-8-methyl-4H-pyrimido[1,2-b]pyridazin-4-one
7-(3-(5-chloro-6-fluoropyridin-3-yl)-7,8-dihydro-1,6-naphthyridin-6(5)-yl)-8-methyl-4H-
pyrimido[1,2-b]pyridazin-4-one
7-(3-(6-fluoro-4-methylpyridin-3-yl)-7,8-dihydro-1,6-naphthyridin-6(5H)-yl)-2-
(methoxymethyl)-8-methyl-4H-pyrimido[1,2-b]pyridazin-4-one
7-(3-(2-fluoro-5-methylpyridin-4-yl)-7,8-dihydro-1,6-naphthyridin-6(5H)-yl)-2-
(methoxymethyl)-8-methyl-4H-pyrimido[1,2-b]pyridazin-4-one
7-(3-(6-chloro-4-(difluoromethyl)pyridin-3-yl)-7,8-dihydro-1,6-naphthyridin-6(5H)-yl)-8-
methyl-4H-pyrimido[1,2-b]pyridazin-4-one
7-(3-(3,4-difluorophenyl)-7,8-dihydro-1,6-naphthyridin-6(5H)-yl)-2-(methoxymethyl)-8-
methyl-4H-pyrimido[1,2-b]pyridazin-4-one
7-(3-(5,6-difluoropyridin-3-yl)-7,8-dihydro-1,6-naphthyridin-6(5H)-yl)-8-methyl-4H-
pyrimido[1,2-b]pyridazin-4-one
7-(3-(5-fluoro-2-methylphenyl)-7,8-dihydro-1,6-naphthyridin-6(5H)-yl)-2-
(methoxymethyl)-8-methyl-4H-pyrimido[1,2-b]pyridazin-4-one
7-(3-(6-chloro-4-methylpyridin-3-yl)-7,8-dihydro-1,6-naphthyridin-6(5H)-yl)-8-methyl-4H-
pyrimido[1,2-b]pyridazin-4-one
7-(3-(6-chloropyridazin-3-yl)-7,8-dihydro-1,6-naphthyridin-6(5H)-yl)-8-methyl-4H-
pyrimido[1,2-b]pyridazin-4-one
2-(methoxymethyl)-8-methyl-7-(3-(2,4,5-trifluorophenyl)-7,8-dihydro-1,6-naphthyridin-
6(5H)-yl)-4H-pyrimido[1,2-b]pyridazin-4-one
7-(3-(2,5-difluorophenyl)-7,8-dihydro-1,6-naphthyridin-6(5H)-yl)-2-(methoxymethyl)-8-
methyl-4H-pyrimido[1,2-b]pyridazin-4-one
2-(methoxymethyl)-8-methyl-7-(3-(o-tolyl)-7,8-dihydro-1,6-naphthyridin-6(5H)-yl)-4H-
pyrimido[1,2-b]pyridazin-4-one
7-(3-(1,5-dimethyl-1H-pyrazol-4-yl)-7,8-dihydro-1,6-naphthyridin-6(5H)-yl)-8,9-dimethyl-
4H-pyrimido[1,2-b]pyridazin-4-one
7-(3-(1-(difluoromethyl)-1H-pyrazol-4-yl)-7,8-dihydro-1,6-naphthyridin-6(5H)-yl)-8,9-
dimethyl-4H-pyrimido[1,2-b]pyridazin-4-one
7-(3-(2-isopropylphenyl)-7,8-dihydro-1,6-naphthyridin-6(5H)-yl)-8,9-dimethyl-4H-
pyrimido[1,2-b]pyridazin-4-one
7-(3-(2,3-difluoro-5-methylphenyl)-7,8-dihydro-1,6-naphthyridin-6(5H)-yl)-8,9-dimethyl-
4H-pyrimido[1,2-b]pyridazin-4-one
7-(3-(2-ethylphenyl)-7,8-dihydro-1,6-naphthyridin-6(5H)-yl)-8,9-dimethyl-4H-
pyrimido[1,2-b]pyridazin-4-one
7-(3-(2-fluorophenyl)-7,8-dihydro-1,6-naphthyridin-6(5H)-yl)-8,9-dimethyl-4H-
pyrimido[1,2-b]pyridazin-4-one
7-(3-(2-methoxyphenyl)-7,8-dihydro-1,6-naphthyridin-6(5H)-yl)-8,9-dimethyl-4H-
pyrimido[1,2-b]pyridazin-4-one
7-(3-(2,5-difluorophenyl)-7,8-dihydro-1,6-naphthyridin-6(5H)-yl)-8,9-dimethyl-4H-
pyrimido[1,2-b]pyridazin-4-one
8,9-dimethyl-7-(3-(2,3,5-trifluorophenyl)-7,8-dihydro-1,6-naphthyridin-6(5H)-yl)-4H-
pyrimido[1,2-b]pyridazin-4-one
7-(3-(2,4-difluorophenyl)-7,8-dihydro-1,6-naphthyridin-6(5H)-yl)-8,9-dimethyl-4H-
pyrimido[1,2-b]pyridazin-4-one
8-methyl-7-(3-(3-methylpyridin-2-yl)-7,8-dihydro-1,6-naphthyridin-6(5H)-yl)-4H-
pyrimido[1,2-b]pyridazin-4-one
8-methyl-7-(3-(pyridin-2-yl)-7,8-dihydro-1,6-naphthyridin-6(5H)-yl)-4H-pyrimido[1,2-
b]pyridazin-4-one
7-(3-(5-fluoro-3-methylpyridin-2-yl)-7,8-dihydro-1,6-naphthyridin-6(5H)-yl)-8-methyl-4H-
pyrimido[1,2-b]pyridazin-4-one
8-methyl-7-(3-(5-methylpyridin-2-yl)-7,8-dihydro-1,6-naphthyridin-6(5H)-yl)-4H-
pyrimido[1,2-b]pyridazin-4-one
8-methyl-7-(3-(pyridazin-3-yl)-7,8-dihydro-1,6-naphthyridin-6(5H)-yl)-4H-pyrimido[1,2-
b]pyridazin-4-one
8-methyl-7-(3-(6-methylpyridazin-3-yl)-7,8-dihydro-1,6-naphthyridin-6(5H)-yl)-4H-
pyrimido[1,2-b]pyridazin-4-one
7-(3-(4-fluoropyridin-2-yl)-7,8-dihydro-1,6-naphthyridin-6(5H)-yl)-8-methyl-4H-
pyrimido[1,2-b]pyridazin-4-one
4-methyl-5-(6-(8-methyl-4-oxo-4H-pyrimido[1,2-b]pyridazin-7-yl)-5,6,7,8-tetrahydro-1,6-
naphthyridin-3-yl)picolinonitrile
8-methyl-7-(3-(4-methylpyridazin-3-yl)-7,8-dihydro-1,6-naphthyridin-6(5H)-yl)-4H-
pyrimido[1,2-b]pyridazin-4-one
7-(3-(6-chloro-4-methylpyridin-3-yl)-7,8-dihydro-1,6-naphthyridin-6(5H)-yl)-2-
(methoxymethyl)-8-methyl-4H-pyrimido[1,2-b]pyridazin-4-one
7-(3-(3-fluorophenyl)-7,8-dihydro-1,6-naphthyridin-6(5H)-yl)-2-(methoxymethyl)-8-
methyl-4H-pyrimido[1,2-b]pyridazin-4-one
2-(methoxymethyl)-8-methyl-7-(3-(4-methylpyridin-3-yl)-7,8-dihydro-1,6-naphthyridin-
6(5H)-yl)-4H-pyrimido[1,2-b]pyridazin-4-one
7-(3-(2-fluorophenyl)-7,8-dihydro-1,6-naphthyridin-6(5H)-yl)-2-(methoxymethyl)-8-
methyl-4H-pyrimido[1,2-b]pyridazin-4-one
2-(methoxymethyl)-8-methyl-7-(3-(2-methylpyridin-4-yl)-7,8-dihydro-1,6-naphthyridin-
6(5H)-yl)-4H-pyrimido[1,2-b]pyridazin-4-one
2-(methoxymethyl)-8-methyl-7-(3-(pyridin-4-yl)-7,8-dihydro-1,6-naphthyridin-6(5H)-yl)-
4H-pyrimido[1,2-b]pyridazin-4-one
2-(methoxymethyl)-8-methyl-7-(3-(2-(trifluoromethoxy)phenyl)-7,8-dihydro-1,6-
naphthyridin-6(5H)-yl)-4H-pyrimido[1,2-b]pyridazin-4-one
7-(3-(1-cyclopropyl-1H-pyrazol-4-yl)-7,8-dihydro-1,6-naphthyridin-6(5H)-yl)-2-
(methoxymethyl)-8-methyl-4H-pyrimido[1,2-b]pyridazin-4-one
2-(methoxymethyl)-8-methyl-7-(3-(pyridin-3-yl)-7,8-dihydro-1,6-naphthyridin-6(5H)-yl)-
4H-pyrimido[1,2-b]pyridazin-4-one
7-(3-(1,5-dimethyl-1H-pyrazol-4-yl)-7,8-dihydro-1,6-naphthyridin-6(5H)-yl)-2-
(methoxymethyl)-8-methyl-4H-pyrimido[1,2-b]pyridazin-4-one
2-(methoxymethyl)-8-methyl-7-(3-(thiophen-2-yl)-7,8-dihydro-1,6-naphthyridin-6(5H)-yl)-
4H-pyrimido[1,2-b]pyridazin-4-one
2-(methoxymethyl)-7-(3-(3-methoxythiophen-2-yl)-7,8-dihydro-1,6-naphthyridin-6(5H)-
yl)-8-methyl-4H-pyrimido[1,2-b]pyridazin-4-one
7-(3-(5-(hydroxymethyl)thiophen-2-yl)-7,8-dihydro-1,6-naphthyridin-6(5H)-yl)-2-
(methoxymethyl)-8-methyl-4H-pyrimido[1,2-b]pyridazin-4-one
2-(methoxymethyl)-8-methyl-7-(3-(thiophen-3-yl)-7,8-dihydro-1,6-naphthyridin-6(5H)-yl)-
4H-pyrimido[1,2-b]pyridazin-4-one
7-(3-(furan-2-yl)-7,8-dihydro-1,6-naphthyridin-6(5H)-yl)-2-(methoxymethyl)-8-methyl-4H-
pyrimido[1,2-b]pyridazin-4-one
2-(methoxymethyl)-8-methyl-7-(3-(5-methylfuran-2-yl)-7,8-dihydro-1,6-naphthyridin-
6(5H)-yl)-4H-pyrimido[1,2-b]pyridazin-4-one
7-(3-(6-(difluoromethoxy)pyridin-3-yl)-7,8-dihydro-1,6-naphthyridin-6(5H)-yl)-2-
(methoxymethyl)-8,9-dimethyl-4H-pyrimido[1,2-b]pyridazin-4-one
7-(3-(6-(difluoromethoxy)pyridin-3-yl)-7,8-dihydro-1,6-naphthyridin-6(5H)-yl)-2-
(methoxymethyl)-8-methyl-4H-pyrimido[1,2-b]pyridazin-4-one
7-(3-(6-(difluoromethyl)-4-methylpyridin-3-yl)-7,8-dihydro-1,6-naphthyridin-6(5H)-yl)-8-
methyl-4H-pyrimido[1,2-b]pyridazin-4-one
(S)-2-(methoxymethyl)-8,9-dimethyl-7-(8-methyl-3-(trifluoromethyl)-7,8-dihydro-1,6-
naphthyridin-6(5H)-yl)-4H-pyrimido[1,2-b]pyridazin-4-one
(R)-2-(methoxymethyl)-8,9-dimethyl-7-(8-methyl-3-(trifluoromethyl)-7,8-dihydro-1,6-
naphthyridin-6(5H)-yl)-4H-pyrimido[1,2-b]pyridazin-4-one
8-methyl-7-(3-(pyridin-3-ylmethyl)-7,8-dihydro-1,6-naphthyridin-6(5H)-yl)-4H-
pyrimido[1,2-b]pyridazin-4-one
7-(3-(6-chloro-4-methylpyridin-3-yl)-7,8-dihydro-1,6-naphthyridin-6(5H)-yl)-2-
(methoxymethyl)-8,9-dimethyl-4H-pyrimido[1,2-b]pyridazin-4-one
7-(3-(6,7-dihydro-4H-pyrazolo[5,1-c][1,4]oxazin-3-yl)-7,8-dihydro-1,6-naphthyridin-
6(5H)-yl)-2-(methoxymethyl)-8,9-dimethyl-4H-pyrimido[1,2-b]pyridazin-4-one
7-(3-(3,5-dimethylpyridin-2-yl)-7,8-dihydro-1,6-naphthyridin-6(5H)-yl)-2-
(methoxymethyl)-8-methyl-4H-pyrimido[1,2-b]pyridazin-4-one
7-(3-(3,5-difluoropyridin-2-yl)-7,8-dihydro-1,6-naphthyridin-6(5H)-yl)-2-(methoxymethyl)-
8-methyl-4H-pyrimido[1,2-b]pyridazin-4-one
7-(3-(4-fluoropyridin-2-yl)-7,8-dihydro-1,6-naphthyridin-6(5H)-yl)-2-(methoxymethyl)-8-
methyl-4H-pyrimido[1,2-b]pyridazin-4-one
2-(methoxymethyl)-7-(3-(4-methoxypyridin-3-yl)-7,8-dihydro-1,6-naphthyridin-6(5H)-yl)-
8-methyl-4H-pyrimido[1,2-b]pyridazin-4-one
(R)-8,9-dimethyl-7-(5-methyl-3-(trifluoromethyl)-7,8-dihydro-1,6-naphthyridin-6(5H)-yl)-
4H-pyrimido[1,2-b]pyridazin-4-one
(S)-8,9-dimethyl-7-(5-methyl-3-(trifluoromethyl)-7,8-dihydro-1,6-naphthyridin-6(5H)-yl)-
4H-pyrimido[1,2-b]pyridazin-4-one
7-(3-(6-methoxy-4-methylpyridin-3-yl)-7,8-dihydro-1,6-naphthyridin-6(5H)-yl)-8-methyl-
4H-pyrimido[1,2-b]pyridazin-4-one
7-(3-(1-acetyl-1,2,3,6-tetrahydropyridin-4-yl)-7,8-dihydro-1,6-naphthyridin-6(5H)-yl)-8-
methyl-4H-pyrimido[1,2-b]pyridazin-4-one
8-methyl-7-(3-(2-methylthiazol-4-yl)-7,8-dihydro-1,6-naphthyridin-6(5H)-yl)-4H-
pyrimido[1,2-b]pyridazin-4-one
7-(3-(1,3-dimethyl-1H-pyrazol-5-yl)-7,8-dihydro-1,6-naphthyridin-6(5H)-yl)-8-methyl-4H-
pyrimido[1,2-b]pyridazin-4-one
7-(3-(1,5-dimethyl-1H-imidazol-2-yl)-7,8-dihydro-1,6-naphthyridin-6(5H)-yl)-8-methyl-
4H-pyrimido[1,2-b]pyridazin-4-one
8-methyl-7-(3-(1-methyl-1H-imidazol-5-yl)-7,8-dihydro-1,6-naphthyridin-6(5H)-yl)-4H-
pyrimido[1,2-b]pyridazin-4-one
8-methyl-7-(3-(5-methylthiazol-4-yl)-7,8-dihydro-1,6-naphthyridin-6(5H)-yl)-4H-
pyrimido[1,2-b]pyridazin-4-one
8-methyl-7-(3-(4-methylthiophen-3-yl)-7,8-dihydro-1,6-naphthyridin-6(5H)-yl)-4H-
pyrimido[1,2-b]pyridazin-4-one
8-methyl-7-(3-(4-methylthiazol-5-yl)-7,8-dihydro-1,6-naphthyridin-6(5H)-yl)-4H-
pyrimido[1,2-b]pyridazin-4-one
7-(3-(1-isopropyl-1H-pyrazol-5-yl)-7,8-dihydro-1,6-naphthyridin-6(5H)-yl)-8-methyl-4H-
pyrimido[1,2-b]pyridazin-4-one
8-methyl-7-(3-(2-methyloxazol-5-yl)-7,8-dihydro-1,6-naphthyridin-6(5H)-yl)-4H-
pyrimido[1,2-b]pyridazin-4-one
7-(3-(6-fluoro-4-methylpyridin-3-yl)-7,8-dihydro-1,6-naphthyridin-6(5H)-yl)-8-methyl-4H-
pyrimido[1,2-b]pyridazin-4-one
7-(3-(5-fluoro-4-methylpyridin-3-yl)-7,8-dihydro-1,6-naphthyridin-6(5H)-yl)-8-methyl-4H-
pyrimido[1,2-b]pyridazin-4-one
8-methyl-7-(3-(4-methyl-6-(trifluoromethyl)pyridin-3-yl)-7,8-dihydro-1,6-naphthyridin-
6(5H)-yl)-4H-pyrimido[1,2-b]pyridazin-4-one
7-(3-(2,4-dimethylpyridin-3-yl)-7,8-dihydro-1,6-naphthyridin-6(5H)-yl)-8-methyl-4H-
pyrimido[1,2-b]pyridazin-4-one
7-(3-(4-acetylpyridin-3-yl)-7,8-dihydro-1,6-naphthyridin-6(5H)-yl)-8-methyl-4H-
pyrimido[1,2-b]pyridazin-4-one
8-methyl-7-(3-(4-methylpyridin-3-yl)-7,8-dihydro-1,6-naphthyridin-6(5H)-yl)-4H-
pyrimido[1,2-b]pyridazin-4-one
7-(3-(6-amino-4-methylpyridin-3-yl)-7,8-dihydro-1,6-naphthyridin-6(5H)-yl)-8-methyl-4H-
pyrimido[1,2-b]pyridazin-4-one
7-(3-(4,6-dimethylpyridin-3-yl)-7,8-dihydro-1,6-naphthyridin-6(5H)-yl)-8-methyl-4H-
pyrimido[1,2-b]pyridazin-4-one
4-methyl-5-(6-(8-methyl-4-oxo-4H-pyrimido[1,2-b]pyridazin-7-yl)-5,6,7,8-tetrahydro-1,6-
naphthyridin-3-yl)picolinic acid
7-(3-(6-(dimethylamino)-4-methylpyridin-3-yl)-7,8-dihydro-1,6-naphthyridin-6(5H)-yl)-8-
methyl-4H-pyrimido[1,2-b]pyridazin-4-one
7-(3-(1,5-dimethyl-1H-pyrazol-4-yl)-7,8-dihydro-1,6-naphthyridin-6(5H)-yl)-8-methyl-4H-
pyrimido[1,2-b]pyridazin-4-one
8-methyl-7-(3-(3-methylthiophen-2-yl)-7,8-dihydro-1,6-naphthyridin-6(5H)-yl)-4/-
pyrimido[1,2-b]pyridazin-4-one
3-(6-(8-methyl-4-oxo-4H-pyrimido[1,2-b]pyridazin-7-yl)-5,6,7,8-tetrahydro-1,6-
naphthyridin-3-yl)thiophene-2-carbonitrile
8-methyl-7-(3-(1-methyl-3-(trifluoromethyl)-1/-pyrazol-4-yl)-7,8-dihydro-1,6-
naphthyridin-6(5H)-yl)-4H-pyrimido[1,2-b]pyridazin-4-one
7-(3-(3-(difluoromethyl)-1-methyl-1H-pyrazol-4-yl)-7,8-dihydro-1,6-naphthyridin-6(5H)-
yl)-8-methyl-4H-pyrimido[1,2-b]pyridazin-4-one
7-(3-(5-(difluoromethyl)-1-methyl-1H-pyrazol-4-yl)-7,8-dihydro-1,6-naphthyridin-6(5H)-
yl)-8-methyl-4H-pyrimido[1,2-b]pyridazin-4-one
7-(3-(1-(difluoromethyl)-5-methyl-1H-pyrazol-4-yl)-7,8-dihydro-1,6-naphthyridin-6(5H)-
yl)-8-methyl-4H-pyrimido[1,2-b]pyridazin-4-one
3-(6-(8-methyl-4-oxo-4H-pyrimido[1,2-b]pyridazin-7-yl)-5,6,7,8-tetrahydro-1,6-
naphthyridin-3-yl)thiophene-2-carbaldehyde
7-(3-(3,5-dimethylisoxazol-4-yl)-7,8-dihydro-1,6-naphthyridin-6(5)-yl)-8-methyl-4H-
pyrimido[1,2-b]pyridazin-4-one
7-(3-(1-(difluoromethyl)-3-methyl-1H-pyrazol-4-yl)-7,8-dihydro-1,6-naphthyridin-6(5H)-
yl)-8-methyl-4H-pyrimido[1,2-b]pyridazin-4-one
7-(3-(2-(2-hydroxypropan-2-yl)pyridin-3-yl)-7,8-dihydro-1,6-naphthyridin-6(5H)-yl)-8-
methyl-4H-pyrimido[1,2-b]pyridazin-4-one
7-(3-(5-(2-hydroxypropan-2-yl)pyridin-3-yl)-7,8-dihydro-1,6-naphthyridin-6(5H)-yl)-8-
methyl-4H-pyrimido[1,2-b]pyridazin-4-one
7-(3-(2,4-dimethylpyrimidin-5-yl)-7,8-dihydro-1,6-naphthyridin-6(5H)-yl)-8-methyl-4H-
pyrimido[1,2-b]pyridazin-4-one
6-methyl-5-(6-(8-methyl-4-oxo-4H-pyrimido[1,2-b]pyridazin-7-yl)-5,6,7,8-tetrahydro-1,6-
naphthyridin-3-yl)picolinonitrile
7-(3-(2-isopropylpyridin-3-yl)-7,8-dihydro-1,6-naphthyridin-6(5H)-yl)-8-methyl-4H-
pyrimido[1,2-b]pyridazin-4-one
1,5-dimethyl-4-(6-(8-methyl-4-oxo-4H-pyrimido[1,2-b]pyridazin-7-yl)-5,6,7,8-tetrahydro-
1,6-naphthyridin-3-yl)-1H-pyrrole-2-carbonitrile
8-methyl-7-(3-(1,3,5-trimethyl-1H-pyrazol-4-yl)-7,8-dihydro-1,6-naphthyridin-6(5H)-yl)-
4H-pyrimido[1,2-b]pyridazin-4-one
7-(3-(2-(difluoromethyl)pyridin-3-yl)-7,8-dihydro-1,6-naphthyridin-6(5H)-yl)-8-methyl-
4H-pyrimido[1,2-b]pyridazin-4-one
7-(3-(6-methoxy-2-methylpyridin-3-yl)-7,8-dihydro-1,6-naphthyridin-6(5H)-yl)-8-methyl-
4H-pyrimido[1,2-b]pyridazin-4-one
7-(3-(2-methoxy-3-methylpyridin-4-yl)-7,8-dihydro-1,6-naphthyridin-6(5H)-yl)-8-methyl-
4H-pyrimido[1,2-b]pyridazin-4-one
N-cyclopropyl-4-methyl-5-(6-(8-methyl-4-oxo-4H-pyrimido[1,2-b]pyridazin-7-yl)-5,6,7,8-
tetrahydro-1,6-naphthyridin-3-yl)thiophene-2-carboxamide
8,9-dimethyl-7-(3-(tetrahydro-2H-pyran-4-yl)-7,8-dihydro-1,6-naphthyridin-6(5H)-yl)-4H-
pyrimido[1,2-b]pyridazin-4-one
7-(3-cyclohexyl-7,8-dihydro-1,6-naphthyridin-6(5H)-yl)-8,9-dimethyl-4H-pyrimido[1,2-
b]pyridazin-4-one
7-(3-(6,7-dihydropyrazolo[1,5-a]pyrimidin-4(5H)-yl)-7,8-dihydro-1,6-naphthyridin-6(5H)-
yl)-8-methyl-4H-pyrimido[1,2-b]pyridazin-4-one
(R)-8-methyl-7-(3-(2-methylmorpholino)-7,8-dihydro-1,6-naphthyridin-6(5H)-yl)-4H-
pyrimido[1,2-b]pyridazin-4-one
7-(3-(6,7-dihydropyrazolo[1,5-a]pyrimidin-4(5H)-yl)-7,8-dihydro-1,6-naphthyridin-6(5H)-
yl)-9-methyl-4H-pyrimido[1,2-b]pyridazin-4-one
3-chloro-7-(3-(6,7-dihydropyrazolo[1,5-a]pyrimidin-4(5H)-yl)-7,8-dihydro-1,6-
naphthyridin-6(5H)-yl)-8-methyl-4H-pyrimido[1,2-b]pyridazin-4-one
8-methyl-7-(3-(2-methyl-6,7-dihydropyrazolo[1,5-a]pyrimidin-4(5H)-yl)-7,8-dihydro-1,6-
naphthyridin-6(5H)-yl)-4H-pyrimido[1,2-b]pyridazin-4-one
7-(3-(1H-pyrazol-1-yl)-7,8-dihydro-1,6-naphthyridin-6(5H)-yl)-8-methyl-4H-pyrimido[1,2-
b]pyridazin-4-one
7-(3-(2,3-dihydro-1H-pyrido[2,3-b][1,4]oxazin-1-yl)-7,8-dihydro-1,6-naphthyridin-6(5H)-
yl)-8-methyl-4H-pyrimido[1,2-b]pyridazin-4-one
8-methyl-7-(3-((1-methyl-1H-pyrazol-5-yl)amino)-7,8-dihydro-1,6-naphthyridin-6(5H)-yl)-
4H-pyrimido[1,2-b]pyridazin-4-one
7-(3-(6,7-dihydropyrazolo[1,5-a]pyrimidin-4(5H)-yl)-7,8-dihydro-1,6-naphthyridin-6(5H)-
yl)-3-fluoro-2,8-dimethyl-4H-pyrimido[1,2-b]pyridazin-4-one
7-(3-(6,7-dihydropyrazolo[1,5-a]pyrimidin-4(5H)-yl)-7,8-dihydro-1,6-naphthyridin-6(5H)-
yl)-2,8-dimethyl-4H-pyrimido[1,2-b]pyridazin-4-one
7-(3-(6,7-dihydropyrazolo[1,5-a]pyrimidin-4(5H)-yl)-7,8-dihydro-1,6-naphthyridin-6(5H)-
yl)-8-methyl-2-(trifluoromethyl)-4H-pyrimido[1,2-b]pyridazin-4-one
7-(3-(6,7-dihydropyrazolo[1,5-a]pyrimidin-4(5H)-yl)-7,8-dihydro-1,6-naphthyridin-6(5H)-
yl)-3-fluoro-2,8,9-trimethyl-4H-pyrimido[1,2-b]pyridazin-4-one
7-(3-(6,7-dihydropyrazolo[1,5-a]pyrimidin-4(5H)-yl)-7,8-dihydro-1,6-naphthyridin-6(5H)-
yl)-2,3,8-trimethyl-4H-pyrimido[1,2-b]pyridazin-4-one
2-(difluoromethyl)-7-(3-(6,7-dihydropyrazolo[1,5-a]pyrimidin-4(5H)-yl)-7,8-dihydro-1,6-
naphthyridin-6(5H)-yl)-8-methyl-4H-pyrimido[1,2-b]pyridazin-4-one
2-cyclopropyl-7-(3-(6,7-dihydropyrazolo[1,5-a]pyrimidin-4(5H)-yl)-7,8-dihydro-1,6-
naphthyridin-6(5H)-yl)-8-methyl-4H-pyrimido[1,2-b]pyridazin-4-one
3-chloro-7-(3-(6,7-dihydropyrazolo[1,5-a]pyrimidin-4(5H)-yl)-7,8-dihydro-1,6-
naphthyridin-6(5H)-yl)-2,8-dimethyl-4H-pyrimido[1,2-b]pyridazin-4-one
7-(3-(2,3-dihydro-1H-pyrido[2,3-b][1,4]oxazin-1-yl)-7,8-dihydro-1,6-naphthyridin-6(5H)-
yl)-2,8-dimethyl-4H-pyrimido[1,2-b]pyridazin-4-one
(R)-2,8-dimethyl-7-(3-(2-methylmorpholino)-7,8-dihydro-1,6-naphthyridin-6(5H)-yl)-4H-
pyrimido[1,2-b]pyridazin-4-one
7-(3-((2-fluoropyridin-3-yl)amino)-7,8-dihydro-1,6-naphthyridin-6(5H)-yl)-2,8-dimethyl-
4H-pyrimido[1,2-b]pyridazin-4-one
2,8-dimethyl-7-(3-((1-methyl-1H-pyrazol-5-yl)amino)-7,8-dihydro-1,6-naphthyridin-6(5H)-
yl)-4H-pyrimido[1,2-b]pyridazin-4-one
2,8-dimethyl-7-(3-(2-methyl-6,7-dihydropyrazolo[1,5-a]pyrimidin-4(5H)-yl)-7,8-dihydro-
1,6-naphthyridin-6(5H)-yl)-4H-pyrimido[1,2-b]pyridazin-4-one
7-(3-(1H-pyrazol-1-yl)-7,8-dihydro-1,6-naphthyridin-6(5H)-yl)-2,8-dimethyl-4H-
pyrimido[1,2-b]pyridazin-4-one
7-(3-(6,7-dihydropyrazolo[1,5-a]pyrimidin-4(5H)-yl)-7,8-dihydro-1,6-naphthyridin-6(5H)-
yl)-2,8,9-trimethyl-4H-pyrimido[1,2-b]pyridazin-4-one
2-(difluoromethyl)-7-(3-(2,2-dimethylmorpholino)-7,8-dihydro-1,6-naphthyridin-6(5H)-yl)-
8-methyl-4H-pyrimido[1,2-b]pyridazin-4-one
(R)-2-(difluoromethyl)-8-methyl-7-(3-(2-methylmorpholino)-7,8-dihydro-1,6-naphthyridin-
6(5H)-yl)-4H-pyrimido[1,2-b]pyridazin-4-one
7-(3-(1H-pyrazol-1-yl)-7,8-dihydro-1,6-naphthyridin-6(5H)-yl)-2-(difluoromethyl)-8-
methyl-4H-pyrimido[1,2-b]pyridazin-4-one
2-(difluoromethyl)-8-methyl-7-(3-((1-methyl-1/-pyrazol-5-yl)amino)-7,8-dihydro-1,6-
naphthyridin-6(5H)-yl)-4H-pyrimido[1,2-b]pyridazin-4-one
2-(difluoromethyl)-8-methyl-7-(3-(2-methyl-6,7-dihydropyrazolo[1,5-a]pyrimidin-4(5H)-
yl)-7,8-dihydro-1,6-naphthyridin-6(5H)-yl)-4H-pyrimido[1,2-b]pyridazin-4-one
2-(difluoromethyl)-7-(3-(2,3-dihydro-1H-pyrido[2,3-b][1,4]oxazin-1-yl)-7,8-dihydro-1,6-
naphthyridin-6(5H)-yl)-8-methyl-4H-pyrimido[1,2-b]pyridazin-4-one
7-(3-(6,7-dihydropyrazolo[1,5-a]pyrimidin-4(5H)-yl)-7,8-dihydro-1,6-naphthyridin-6(5H)-
yl)-8,9-dimethyl-2-(trifluoromethyl)-4H-pyrimido[1,2-b]pyridazin-4-one
2-(difluoromethyl)-7-(3-(6,7-dihydropyrazolo[1,5-a]pyrimidin-4(5H)-yl)-7,8-dihydro-1,6-
naphthyridin-6(5H)-yl)-8,9-dimethyl-4H-pyrimido[1,2-b]pyridazin-4-one
(R)-8,9-dimethyl-7-(3-(2-methylmorpholino)-7,8-dihydro-1,6-naphthyridin-6(5H)-yl)-4H-
pyrimido[1,2-b]pyridazin-4-one
7-(3-(2,3-dihydro-1H-pyrido[2,3-b][1,4]oxazin-1-yl)-7,8-dihydro-1,6-naphthyridin-6(5H)-
yl)-8,9-dimethyl-4H-pyrimido[1,2-b]pyridazin-4-one
7-(3-(1H-pyrazol-1-yl)-7,8-dihydro-1,6-naphthyridin-6(5H)-yl)-8,9-dimethyl-4H-
pyrimido[1,2-b]pyridazin-4-one
8,9-dimethyl-7-(3-(2-methyl-6,7-dihydropyrazolo[1,5-a]pyrimidin-4(5H)-yl)-7,8-dihydro-
1,6-naphthyridin-6(5H)-yl)-4H-pyrimido[1,2-b]pyridazin-4-one
7-(3-(6,7-dihydropyrazolo[1,5-a]pyrimidin-4(5H)-yl)-7,8-dihydro-1,6-naphthyridin-6(5H)-
yl)-8,9-dimethyl-4H-pyrimido[1,2-b]pyridazin-4-one
7-(3-(2-oxa-5-azabicyclo[2.2.1]heptan-5-yl)-7,8-dihydro-1,6-naphthyridin-6(5H)-yl)-2,8,9-
trimethyl-4H-pyrimido[1,2-b]pyridazin-4-one
7-(3-((2R,6S)-2,6-dimethylmorpholino)-7,8-dihydro-1,6-naphthyridin-6(5H)-yl)-2,8,9-
trimethyl-4H-pyrimido[1,2-b]pyridazin-4-one
7-(3-(2,2-dimethylmorpholino)-7,8-dihydro-1,6-naphthyridin-6(5H)-yl)-2,8,9-trimethyl-4H-
pyrimido[1,2-b]pyridazin-4-one
2-(difluoromethyl)-7-(3-(2,2-dimethylmorpholino)-7,8-dihydro-1,6-naphthyridin-6(5H)-yl)-
8,9-dimethyl-4H-pyrimido[1,2-b]pyridazin-4-one
2-(difluoromethyl)-7-(3-((2R,6S)-2,6-dimethylmorpholino)-7,8-dihydro-1,6-naphthyridin-
6(5H)-yl)-8,9-dimethyl-4H-pyrimido[1,2-b]pyridazin-4-one
7-(3-(2-oxa-5-azabicyclo[2.2.1]heptan-5-yl)-7,8-dihydro-1,6-naphthyridin-6(5/)-yl)-2,8-
dimethyl-4H-pyrimido[1,2-b]pyridazin-4-one
7-(3-(8-oxa-3-azabicyclo[3.2.1]octan-3-yl)-7,8-dihydro-1,6-naphthyridin-6(5H)-yl)-2,8,9-
trimethyl-4H-pyrimido[1,2-b]pyridazin-4-one
2,8,9-trimethyl-7-(3-(((tetrahydro-2H-pyran-3-yl)methyl)amino)-7,8-dihydro-1,6-
naphthyridin-6(5H)-yl)-4H-pyrimido[1,2-b]pyridazin-4-one
7-(3-(4-methoxypiperidin-1-yl)-7,8-dihydro-1,6-naphthyridin-6(5H)-yl)-2,8,9-trimethyl-
4H-pyrimido[1,2-b]pyridazin-4-one
2,8,9-trimethyl-7-(3-thiomorpholino-7,8-dihydro-1,6-naphthyridin-6(5H)-yl)-4H-
pyrimido[1,2-b]pyridazin-4-one
7-(3-(2-oxa-7-azaspiro[3.5]nonan-7-yl)-7,8-dihydro-1,6-naphthyridin-6(5H)-yl)-2,8,9-
trimethyl-4H-pyrimido[1,2-b]pyridazin-4-one
2-(difluoromethyl)-7-(3-(4,4-difluoropiperidin-1-yl)-7,8-dihydro-1,6-naphthyridin-6(5H)-
yl)-8,9-dimethyl-4H-pyrimido[1,2-b]pyridazin-4-one
2,8,9-trimethyl-7-(3-(2-(pyridin-3-yl)morpholino)-7,8-dihydro-1,6-naphthyridin-6(5H)-yl)-
4H-pyrimido[1,2-b]pyridazin-4-one
2,8,9-trimethyl-7-(3-(((2-methylthiazol-4-yl)methyl)amino)-7,8-dihydro-1,6-naphthyridin-
6(5H)-yl)-4H-pyrimido[1,2-b]pyridazin-4-one
7-(3-(2,2-dimethyl-2,3-dihydro-4H-benzo[b][1,4]oxazin-4-yl)-7,8-dihydro-1,6-
naphthyridin-6(5H)-yl)-8-methyl-4H-pyrimido[1,2-b]pyridazin-4-one
7-(3-(7-fluoro-3-methyl-2,3-dihydro-4H-benzo[b][1,4]oxazin-4-yl)-7,8-dihydro-1,6-
naphthyridin-6(5H)-yl)-8-methyl-4H-pyrimido[1,2-b]pyridazin-4-one
8-methyl-7-(3-(6-methyl-2,3-dihydro-4H-benzo[b][1,4]oxazin-4-yl)-7,8-dihydro-1,6-
naphthyridin-6(5H)-yl)-4H-pyrimido[1,2-b]pyridazin-4-one
7-(3-(3,6-dimethyl-2,3-dihydro-4H-benzo[b][1,4]oxazin-4-yl)-7,8-dihydro-1,6-
naphthyridin-6(5H)-yl)-8-methyl-4H-pyrimido[1,2-b]pyridazin-4-one
7-(3-(7-fluoro-2-methyl-2,3-dihydro-4H-benzo[b][1,4]oxazin-4-yl)-7,8-dihydro-1,6-
naphthyridin-6(5H)-yl)-8-methyl-4H-pyrimido[1,2-b]pyridazin-4-one
7-(3-(7-fluoro-2,3-dihydro-4H-benzo[b][1,4]oxazin-4-yl)-7,8-dihydro-1,6-naphthyridin-
6(5H)-yl)-8-methyl-4H-pyrimido[1,2-b]pyridazin-4-one
8-methyl-7-(3-(2-methyl-2,3-dihydro-4H-benzo[b][1,4]oxazin-4-yl)-7,8-dihydro-1,6-
naphthyridin-6(5H)-yl)-4H-pyrimido[1,2-b]pyridazin-4-one
7-(3-(2,3-dihydro-1H-pyrrolo[3,2-b]pyridin-1-yl)-7,8-dihydro-1,6-naphthyridin-6(5H)-yl)-
8-methyl-4H-pyrimido[1,2-b]pyridazin-4-one
7-(3-(2-(2-fluorophenyl)morpholino)-7,8-dihydro-1,6-naphthyridin-6(5H)-yl)-8-methyl-4H-
pyrimido[1,2-b]pyridazin-4-one
8-methyl-7-(3-(phenylamino)-7,8-dihydro-1,6-naphthyridin-6(5H)-yl)-4H-pyrimido[1,2-
b]pyridazin-4-one
7-(3-(2-(3-fluorophenyl)morpholino)-7,8-dihydro-1,6-naphthyridin-6(5H)-yl)-8-methyl-4H-
pyrimido[1,2-b]pyridazin-4-one
7-(3-((2-methoxyphenyl)amino)-7,8-dihydro-1,6-naphthyridin-6(5H)-yl)-8-methyl-4H-
pyrimido[1,2-b]pyridazin-4-one
7-(3-((4-fluorophenyl)amino)-7,8-dihydro-1,6-naphthyridin-6(5H)-yl)-8-methyl-4H-
pyrimido[1,2-b]pyridazin-4-one
7-(3-(2-(4-fluorophenyl)morpholino)-7,8-dihydro-1,6-naphthyridin-6(5H)-yl)-8-methyl-4H-
pyrimido[1,2-b]pyridazin-4-one
7-(3-((4-fluoro-2-methylphenyl)amino)-7,8-dihydro-1,6-naphthyridin-6(5)-yl)-8-methyl-
4H-pyrimido[1,2-b]pyridazin-4-one
8-methyl-7-(3-(2-phenylmorpholino)-7,8-dihydro-1,6-naphthyridin-6(5H)-yl)-4H-
pyrimido[1,2-b]pyridazin-4-one
7-(3-((2-chlorophenyl)amino)-7,8-dihydro-1,6-naphthyridin-6(5H)-yl)-8-methyl-4H-
pyrimido[1,2-b]pyridazin-4-one
7-(3-(2,2-dimethylmorpholino)-7,8-dihydro-1,6-naphthyridin-6(5H)-yl)-8-methyl-4H-
pyrimido[1,2-b]pyridazin-4-one
7-(3-((5-fluoro-2-methylphenyl)amino)-7,8-dihydro-1,6-naphthyridin-6(5H)-yl)-8-methyl-
4H-pyrimido[1,2-b]pyridazin-4-one
7-(3-((2-fluoro-5-methoxyphenyl)amino)-7,8-dihydro-1,6-naphthyridin-6(5H)-yl)-8-
methyl-4H-pyrimido[1,2-b]pyridazin-4-one
7-(3-((3-(difluoromethoxy)phenyl)amino)-7,8-dihydro-1,6-naphthyridin-6(5H)-yl)-8-
methyl-4H-pyrimido[1,2-b]pyridazin-4-one
7-(3-((2,3-dihydrobenzo[b][1,4]dioxin-5-yl)amino)-7,8-dihydro-1,6-naphthyridin-6(5H)-
yl)-8-methyl-4H-pyrimido[1,2-b]pyridazin-4-one
(S)-8-methyl-7-(3-(2-phenylmorpholino)-7,8-dihydro-1,6-naphthyridin-6(5H)-yl)-4H-
pyrimido[1,2-b]pyridazin-4-one
7-(3-((4-fluoro-2-methoxyphenyl)amino)-7,8-dihydro-1,6-naphthyridin-6(5H)-yl)-8-
methyl-4H-pyrimido[1,2-b]pyridazin-4-one
8-methyl-7-(3-(o-tolylamino)-7,8-dihydro-1,6-naphthyridin-6(5H)-yl)-4H-pyrimido[1,2-
b]pyridazin-4-one
8-methyl-7-(3-(2-(pyridin-2-yl)morpholino)-7,8-dihydro-1,6-naphthyridin-6(5H)-yl)-4H-
pyrimido[1,2-b]pyridazin-4-one
8-methyl-7-(3-(2-(pyridin-3-yl)morpholino)-7,8-dihydro-1,6-naphthyridin-6(5H)-yl)-4H-
pyrimido[1,2-b]pyridazin-4-one
7-(3-((2-fluoropyridin-3-yl)amino)-7,8-dihydro-1,6-naphthyridin-6(5H)-yl)-8-methyl-4H-
pyrimido[1,2-b]pyridazin-4-one
7-(3-((3-fluorophenyl)amino)-7,8-dihydro-1,6-naphthyridin-6(5H)-yl)-8-methyl-4H-
pyrimido[1,2-b]pyridazin-4-one
3-((6-(8-methyl-4-oxo-4H-pyrimido[1,2-b]pyridazin-7-yl)-5,6,7,8-tetrahydro-1,6-
naphthyridin-3-yl)amino)benzonitrile
4-fluoro-2-((6-(8-methyl-4-oxo-4H-pyrimido[1,2-b]pyridazin-7-yl)-5,6,7,8-tetrahydro-1,6-
naphthyridin-3-yl)amino)benzonitrile
7-(3-((3-chloro-5-methoxyphenyl)amino)-7,8-dihydro-1,6-naphthyridin-6(5H)-yl)-8-
methyl-4H-pyrimido[1,2-b]pyridazin-4-one
8-methyl-7-(3-((3-(2-methylthiazol-4-yl)phenyl)amino)-7,8-dihydro-1,6-naphthyridin-
6(5H)-yl)-4H-pyrimido[1,2-b]pyridazin-4-one
7-(3-(2-(3-methoxyphenyl)morpholino)-7,8-dihydro-1,6-naphthyridin-6(5H)-yl)-8-methyl-
4H-pyrimido[1,2-b]pyridazin-4-one
7-(3-(indolin-1-yl)-7,8-dihydro-1,6-naphthyridin-6(5H)-yl)-8-methyl-4/-pyrimido[1,2-
b]pyridazin-4-one
8-methyl-7-(3-(((tetrahydro-2H-pyran-3-yl)methyl)amino)-7,8-dihydro-1,6-naphthyridin-
6(5H)-yl)-4H-pyrimido[1,2-b]pyridazin-4-one
7-(3-((6-fluoropyridin-3-yl)amino)-7,8-dihydro-1,6-naphthyridin-6(5H)-yl)-8-methyl-4H-
pyrimido[1,2-b]pyridazin-4-one
8-methyl-7-(3-(pyridin-3-ylamino)-7,8-dihydro-1,6-naphthyridin-6(5H)-yl)-4H-
pyrimido[1,2-b]pyridazin-4-one
7-(3-((cyclopropylmethyl)amino)-7,8-dihydro-1,6-naphthyridin-6(5H)-yl)-8-methyl-4/-
pyrimido[1,2-b]pyridazin-4-one
7-(3-(isobutylamino)-7,8-dihydro-1,6-naphthyridin-6(5H)-yl)-8-methyl-4H-pyrimido[1,2-
b]pyridazin-4-one
8-methyl-7-(3-(((tetrahydro-2H-pyran-4-yl)methyl)amino)-7,8-dihydro-1,6-naphthyridin-
6(5H)-yl)-4H-pyrimido[1,2-b]pyridazin-4-one
7-(3-(diethylamino)-7,8-dihydro-1,6-naphthyridin-6(5H)-yl)-8-methyl-4H-pyrimido[1,2-
b]pyridazin-4-one
8-methyl-7-(3-(2-(pyridin-4-yl)morpholino)-7,8-dihydro-1,6-naphthyridin-6(5H)-yl)-4H-
pyrimido[1,2-b]pyridazin-4-one
7-(3-((3-fluoro-2-methylphenyl)amino)-7,8-dihydro-1,6-naphthyridin-6(5/)-yl)-8-methyl-
4H-pyrimido[1,2-b]pyridazin-4-one
7-(3-(2-(4-methoxyphenyl)morpholino)-7,8-dihydro-1,6-naphthyridin-6(5H)-yl)-8-methyl-
4H-pyrimido[1,2-b]pyridazin-4-one
(R)-8-methyl-7-(3-(2-phenylmorpholino)-7,8-dihydro-1,6-naphthyridin-6(5H)-yl)-4H-
pyrimido[1,2-b]pyridazin-4-one
8-methyl-7-(3-(pyridin-4-ylamino)-7,8-dihydro-1,6-naphthyridin-6(5H)-yl)-4H-
pyrimido[1,2-b]pyridazin-4-one
7-(3-((2-fluorophenyl)amino)-7,8-dihydro-1,6-naphthyridin-6(5H)-yl)-8-methyl-4H-
pyrimido[1,2-b]pyridazin-4-one
7-(3-(2-benzylmorpholino)-7,8-dihydro-1,6-naphthyridin-6(5H)-yl)-8-methyl-4H-
pyrimido[1,2-b]pyridazin-4-one
7-(3-((2,6-difluoropyridin-4-yl)amino)-7,8-dihydro-1,6-naphthyridin-6(5H)-yl)-8-methyl-
4H-pyrimido[1,2-b]pyridazin-4-one
7-(3-((6-isopropylpyridin-3-yl)amino)-7,8-dihydro-1,6-naphthyridin-6(5H)-yl)-8-methyl-
4H-pyrimido[1,2-b]pyridazin-4-one
8-methyl-7-(3-((3-methylpyridin-4-yl)amino)-7,8-dihydro-1,6-naphthyridin-6(5H)-yl)-4H-
pyrimido[1,2-b]pyridazin-4-one
7-(3-(6-fluoro-3,4-dihydroquinolin-1(2H)-yl)-7,8-dihydro-1,6-naphthyridin-6(5H)-yl)-8-
methyl-4H-pyrimido[1,2-b]pyridazin-4-one
3-fluoro-5-((6-(8-methyl-4-oxo-4H-pyrimido[1,2-b]pyridazin-7-yl)-5,6,7,8-tetrahydro-1,6-
naphthyridin-3-yl)amino)benzonitrile
7-(3-((3,5-difluorophenyl)amino)-7,8-dihydro-1,6-naphthyridin-6(5H)-yl)-8-methyl-4H-
pyrimido[1,2-b]pyridazin-4-one
7-(3-((2,3-difluorophenyl)amino)-7,8-dihydro-1,6-naphthyridin-6(5H)-yl)-8-methyl-4H-
pyrimido[1,2-b]pyridazin-4-one
7-(3-((6-fluoropyridin-2-yl)amino)-7,8-dihydro-1,6-naphthyridin-6(5/)-yl)-8-methyl-4H-
pyrimido[1,2-b]pyridazin-4-one
N-(6-(8-methyl-4-oxo-4H-pyrimido[1,2-b]pyridazin-7-yl)-5,6,7,8-tetrahydro-1,6-
naphthyridin-3-yl)cyclopropanecarboxamide
N-(6-(8-methyl-4-oxo-4HI-pyrimido[1,2-b]pyridazin-7-yl)-5,6,7,8-tetrahydro-1,6-
naphthyridin-3-yl)oxetane-3-carboxamide
N-(6-(8-methyl-4-oxo-4H-pyrimido[1,2-b]pyridazin-7-yl)-5,6,7,8-tetrahydro-1,6-
naphthyridin-3-yl)tetrahydro-2H-pyran-4-carboxamide
7-(3-((3-fluoropyridin-4-yl)amino)-7,8-dihydro-1,6-naphthyridin-6(5H)-yl)-8-methyl-4H-
pyrimido[1,2-b]pyridazin-4-one
7-(3-((3-methoxypyridin-4-yl)amino)-7,8-dihydro-1,6-naphthyridin-6(5H)-yl)-8-methyl-
4H-pyrimido[1,2-b]pyridazin-4-one
7-(3-((3-chloropyridin-4-yl)amino)-7,8-dihydro-1,6-naphthyridin-6(5H)-yl)-8-methyl-4H-
pyrimido[1,2-b]pyridazin-4-one
7-(3-((4-fluoro-2-methylpyridin-3-yl)amino)-7,8-dihydro-1,6-naphthyridin-6(5H)-yl)-8-
methyl-4H-pyrimido[1,2-b]pyridazin-4-one
7-(3-((2-fluoro-6-methylpyridin-3-yl)amino)-7,8-dihydro-1,6-naphthyridin-6(5H)-yl)-8-
methyl-4H-pyrimido[1,2-b]pyridazin-4-one
7-(3-((5-chloro-2-fluoropyridin-3-yl)amino)-7,8-dihydro-1,6-naphthyridin-6(5H)-yl)-8-
methyl-4H-pyrimido[1,2-b]pyridazin-4-one
N-(6-(8-methyl-4-oxo-4H-pyrimido[1,2-b]pyridazin-7-yl)-5,6,7,8-tetrahydro-1,6-
naphthyridin-3-yl)methanesulfonamide
N-(6-(8-methyl-4-oxo-4H-pyrimido[1,2-b]pyridazin-7-yl)-5,6,7,8-tetrahydro-1,6-
naphthyridin-3-yl)cyclopropanesulfonamide
2-methyl-N-(6-(8-methyl-4-oxo-4H-pyrimido[1,2-b]pyridazin-7-yl)-5,6,7,8-tetrahydro-1,6-
naphthyridin-3-yl)thiazole-4-sulfonamide
3,3-difluoro-N-(6-(8-methyl-4-oxo-4H-pyrimido[1,2-b]pyridazin-7-yl)-5,6,7,8-tetrahydro-
1,6-naphthyridin-3-yl)cyclobutane-1-carboxamide
1-methyl-N-(6-(8-methyl-4-oxo-4H/-pyrimido[1,2-b]pyridazin-7-yl)-5,6,7,8-tetrahydro-1,6-
naphthyridin-3-yl)cyclobutane-1-carboxamide
N-(6-(8-methyl-4-oxo-4H-pyrimido[1,2-b]pyridazin-7-yl)-5,6,7,8-tetrahydro-1,6-
naphthyridin-3-yl)oxazole-2-carboxamide
7-(3-((2-fluoropyridin-4-yl)amino)-7,8-dihydro-1,6-naphthyridin-6(5H)-yl)-8-methyl-4H-
pyrimido[1,2-b]pyridazin-4-one
8-methyl-7-(3-((4-methylpyridin-3-yl)amino)-7,8-dihydro-1,6-naphthyridin-6(5H)-yl)-4H-
pyrimido[1,2-b]pyridazin-4-one
7-(3-((6-cyclopropylpyridin-3-yl)amino)-7,8-dihydro-1,6-naphthyridin-6(5H)-yl)-8-methyl-
4H-pyrimido[1,2-b]pyridazin-4-one
7-(3-((6-chloropyridin-3-yl)amino)-7,8-dihydro-1,6-naphthyridin-6(5H)-yl)-8-methyl-4H-
pyrimido[1,2-b]pyridazin-4-one
8-methyl-7-(3-(pyrazin-2-ylamino)-7,8-dihydro-1,6-naphthyridin-6(5H)-yl)-4/-
pyrimido[1,2-b]pyridazin-4-one
7-(3-((5-fluoropyridin-3-yl)amino)-7,8-dihydro-1,6-naphthyridin-6(5H)-yl)-8-methyl-4H-
pyrimido[1,2-b]pyridazin-4-one
8-methyl-7-(3-(((2-methyloxazol-5-yl)methyl)amino)-7,8-dihydro-1,6-naphthyridin-6(5H)-
yl)-4H-pyrimido[1,2-b]pyridazin-4-one
8-methyl-7-(3-((thiazol-4-ylmethyl)amino)-7,8-dihydro-1,6-naphthyridin-6(5H)-yl)-4H-
pyrimido[1,2-b]pyridazin-4-one
8-methyl-7-(3-(((1-methyl-1H-imidazol-5-yl)methyl)amino)-7,8-dihydro-1,6-naphthyridin-
6(5H)-yl)-4H-pyrimido[1,2-b]pyridazin-4-one
7-(3-((6-chloro-5-methylpyridin-3-yl)amino)-7,8-dihydro-1,6-naphthyridin-6(5H)-yl)-8-
methyl-4H-pyrimido[1,2-b]pyridazin-4-one
8-methyl-7-(3-((5-methylpyridin-3-yl)amino)-7,8-dihydro-1,6-naphthyridin-6(5H)-yl)-4H-
pyrimido[1,2-b]pyridazin-4-one
7-(3-((6-methoxypyridin-3-yl)amino)-7,8-dihydro-1,6-naphthyridin-6(5H)-yl)-8-methyl-
4H-pyrimido[1,2-b]pyridazin-4-one
7-(3-((4-chloropyridin-3-yl)amino)-7,8-dihydro-1,6-naphthyridin-6(5H)-yl)-8-methyl-4H-
pyrimido[1,2-b]pyridazin-4-one
8-methyl-7-(3-(pyridazin-4-ylamino)-7,8-dihydro-1,6-naphthyridin-6(5H)-yl)-4H-
pyrimido[1,2-b]pyridazin-4-one
7-(3-((5-methoxypyridin-3-yl)amino)-7,8-dihydro-1,6-naphthyridin-6(5H)-yl)-8-methyl-
4H-pyrimido[1,2-b]pyridazin-4-one
8-methyl-7-(3-((5-(trifluoromethyl)pyridin-3-yl)amino)-7,8-dihydro-1,6-naphthyridin-
6(5H)-yl)-4H-pyrimido[1,2-b]pyridazin-4-one
8-methyl-7-(3-((4-methylpyrimidin-5-yl)amino)-7,8-dihydro-1,6-naphthyridin-6(5H)-yl)-
4H-pyrimido[1,2-b]pyridazin-4-one
7-(3-((4-methoxypyrimidin-2-yl)amino)-7,8-dihydro-1,6-naphthyridin-6(5H)-yl)-8-methyl-
4H-pyrimido[1,2-b]pyridazin-4-one
7-(3-((2-methoxypyridin-4-yl)amino)-7,8-dihydro-1,6-naphthyridin-6(5H)-yl)-8-methyl-
4H-pyrimido[1,2-b]pyridazin-4-one
7-(3-((5-chloropyridin-3-yl)amino)-7,8-dihydro-1,6-naphthyridin-6(5H)-yl)-8-methyl-4H-
pyrimido[1,2-b]pyridazin-4-one
7-(3-((6-fluoro-2-methylpyridin-3-yl)amino)-7,8-dihydro-1,6-naphthyridin-6(5H)-yl)-8-
methyl-4H-pyrimido[1,2-b]pyridazin-4-one
3-(6-(8,9-dimethyl-4-oxo-4H-pyrimido[1,2-b]pyridazin-7-yl)-5,6,7,8-tetrahydro-1,6-
naphthyridin-3-yl)oxazolidin-2-one
7-(3-(4,4-dimethyl-2-oxopyrrolidin-1-yl)-7,8-dihydro-1,6-naphthyridin-6(5H)-yl)-8,9-
dimethyl-4H-pyrimido[1,2-b]pyridazin-4-one
7-(3-(1,1-dioxidoisothiazolidin-2-yl)-7,8-dihydro-1,6-naphthyridin-6(5H)-yl)-8,9-dimethyl-
4H-pyrimido[1,2-b]pyridazin-4-one
8,9-dimethyl-7-(3-((1-oxidotetrahydro-126-thiophen-1-ylidene)amino)-7,8-dihydro-1,6-
naphthyridin-6(5H)-yl)-4H-pyrimido[1,2-b]pyridazin-4-one
7-(3-(3,3-dimethyl-2-oxopyrrolidin-1-yl)-7,8-dihydro-1,6-naphthyridin-6(5H)-yl)-8,9-
dimethyl-4H-pyrimido[1,2-b]pyridazin-4-one
8,9-dimethyl-7-(3-(2-oxo-2,3-dihydro-1H-pyrrolo[3,2-b]pyridin-1-yl)-7,8-dihydro-1,6-
naphthyridin-6(5H)-yl)-4H-pyrimido[1,2-b]pyridazin-4-one
7-(3-(2-fluorophenoxy)-7,8-dihydro-1,6-naphthyridin-6(5H)-yl)-8-methyl-4H-
pyrimido[1,2-b]pyridazin-4-one
7-(3-(2-fluorophenoxy)-7,8-dihydro-1,6-naphthyridin-6(5H)-yl)-2,8-dimethyl-4H-
pyrimido[1,2-b]pyridazin-4-one
7-(3-(2-fluorophenoxy)-7,8-dihydro-1,6-naphthyridin-6(5H)-yl)-8,9-dimethyl-4H-
pyrimido[1,2-b]pyridazin-4-one
3-(2-((6-(8,9-dimethyl-4-oxo-4H-pyrimido[1,2-b]pyridazin-7-yl)-5,6,7,8-tetrahydro-1,6-
naphthyridin-3-yl)oxy)ethyl)oxazolidin-2-one
7-(3-(dimethylphosphoryl)-7,8-dihydro-1,6-naphthyridin-6(5H)-yl)-8,9-dimethyl-4H-
pyrimido[1,2-b]pyridazin-4-one
8-methyl-7-(3-(o-tolyl)-7,8-dihydro-1,6-naphthyridin-6(5/)-yl)-4HI-pyrimido[1,2-
b]pyridazin-4-one
7-(3-(5-fluoro-2-methylphenyl)-7,8-dihydro-1,6-naphthyridin-6(5H)-yl)-8-methyl-4H-
pyrimido[1,2-b]pyridazin-4-one
7-(3-(2-(1,1-difluoroethyl)phenyl)-7,8-dihydro-1,6-naphthyridin-6(5H)-yl)-8-methyl-4H-
pyrimido[1,2-b]pyridazin-4-one
7-(3-(2-(difluoromethyl)phenyl)-7,8-dihydro-1,6-naphthyridin-6(5H)-yl)-8-methyl-4H-
pyrimido[1,2-b]pyridazin-4-one
8-methyl-7-(3-(3-methyl-1H-indol-4-yl)-7,8-dihydro-1,6-naphthyridin-6(5H)-yl)-4H-
pyrimido[1,2-b]pyridazin-4-one
7-(3-(2,3-dihydro-1H-inden-4-yl)-7,8-dihydro-1,6-naphthyridin-6(5H)-yl)-8-methyl-4H-
pyrimido[1,2-b]pyridazin-4-one
2-methyl-3-(6-(8-methyl-4-oxo-4H-pyrimido[1,2-b]pyridazin-7-yl)-5,6,7,8-tetrahydro-1,6-
naphthyridin-3-yl)benzonitrile
1-(2-(6-(8-methyl-4-oxo-4H-pyrimido[1,2-b]pyridazin-7-yl)-5,6,7,8-tetrahydro-1,6-
naphthyridin-3-yl)phenyl)cyclopropane-1-carbonitrile
7-(3-(2-isopropylphenyl)-7,8-dihydro-1,6-naphthyridin-6(5H)-yl)-8-methyl-4H-
pyrimido[1,2-b]pyridazin-4-one
7-(3-(2-cyclopropylphenyl)-7,8-dihydro-1,6-naphthyridin-6(5H)-yl)-8-methyl-4/-
pyrimido[1,2-b]pyridazin-4-one
7-(3-(3-chloro-2-methylphenyl)-7,8-dihydro-1,6-naphthyridin-6(5H)-yl)-8-methyl-4H-
pyrimido[1,2-b]pyridazin-4-one
7-(3-(2,3-dimethylphenyl)-7,8-dihydro-1,6-naphthyridin-6(5H)-yl)-8-methyl-4H-
pyrimido[1,2-b]pyridazin-4-one
7-(3-(4-fluoro-2-methylphenyl)-7,8-dihydro-1,6-naphthyridin-6(5H)-yl)-8-methyl-4H-
pyrimido[1,2-b]pyridazin-4-one
7-(3-(3,5-difluoro-4-methoxy-2-methylphenyl)-7,8-dihydro-1,6-naphthyridin-6(5H)-yl)-8-
methyl-4H-pyrimido[1,2-b]pyridazin-4-one
7-(3-(4,5-difluoro-2-methylphenyl)-7,8-dihydro-1,6-naphthyridin-6(5H)-yl)-8-methyl-4H-
pyrimido[1,2-b]pyridazin-4-one
7-(3-(4,5-difluoro-2-methylphenyl)-7,8-dihydro-1,6-naphthyridin-6(5H)-yl)-8,9-dimethyl-
4H-pyrimido[1,2-b]pyridazin-4-one
7-(3-(3,5-difluoro-2-methylphenyl)-7,8-dihydro-1,6-naphthyridin-6(5H)-yl)-8-methyl-4H-
pyrimido[1,2-b]pyridazin-4-one
8-methyl-7-(2-methyl-7,8-dihydro-1,6-naphthyridin-6(5H)-yl)-4H-pyrimido[1,2-
b]pyridazin-4-one
8-methyl-7-(3-(1-methyl-1H-pyrazol-5-yl)-7,8-dihydro-1,6-naphthyridin-6(5H)-yl-5,5,7,7-
d4)-4H-pyrimido[1,2-b]pyridazin-4-one
7-(3-(3,5-dimethylisoxazol-4-yl)-7,8-dihydro-1,6-naphthyridin-6(5H)-yl-5,5,7,7-d4)-8-
methyl-4H-pyrimido[1,2-b]pyridazin-4-one
3-(6-(8,9-dimethyl-4-oxo-4H-pyrimido[1,2-b]pyridazin-7-yl)-5,6,7,8-tetrahydro-1,6-
naphthyridin-3-yl)-5,5-dimethyloxazolidin-2-one
7-(3-(2-oxa-6-azaspiro[3.3]heptan-6-yl)-7,8-dihydro-1,6-naphthyridin-6(5H)-yl)-8-methyl-
4H-pyrimido[1,2-b]pyridazin-4-one
7-(3-((5-fluoro-2-(trifluoromethyl)pyridin-4-yl)amino)-7,8-dihydro-1,6-naphthyridin-
6(5H)-yl)-8-methyl-4H-pyrimido[1,2-b]pyridazin-4-one
7-(3-((3-fluoro-2-methylpyridin-4-yl)amino)-7,8-dihydro-1,6-naphthyridin-6(5H)-yl)-8-
methyl-4H-pyrimido[1,2-b]pyridazin-4-one
7-(3-((3,5-difluoropyridin-4-yl)amino)-7,8-dihydro-1,6-naphthyridin-6(5H)-yl)-8-methyl-
4H-pyrimido[1,2-b]pyridazin-4-one
7-(3-((5-fluoro-2-methylpyridin-4-yl)amino)-7,8-dihydro-1,6-naphthyridin-6(5H)-yl)-8-
methyl-4H-pyrimido[1,2-b]pyridazin-4-one
3-fluoro-7-(3-((3-fluoropyridin-4-yl)amino)-7,8-dihydro-1,6-naphthyridin-6(5H)-yl)-2,8-
dimethyl-4H-pyrimido[1,2-b]pyridazin-4-one
3-chloro-7-(3-((3-fluoropyridin-4-yl)amino)-7,8-dihydro-1,6-naphthyridin-6(5H)-yl)-8-
methyl-4H-pyrimido[1,2-b]pyridazin-4-one
7-(3-((3-fluoropyridin-4-yl)amino)-7,8-dihydro-1,6-naphthyridin-6(5H)-yl)-2,3,8-trimethyl-
4H-pyrimido[1,2-b]pyridazin-4-one
2-(difluoromethyl)-7-(3-((3-fluoropyridin-4-yl)amino)-7,8-dihydro-1,6-naphthyridin-6(5H)-
yl)-8-methyl-4H-pyrimido[1,2-b]pyridazin-4-one
7-(3-((3-fluoropyridin-4-yl)amino)-7,8-dihydro-1,6-naphthyridin-6(5H)-yl)-8-methyl-2-
(trifluoromethyl)-4H-pyrimido[1,2-b]pyridazin-4-one
3-chloro-7-(3-((3-fluoropyridin-4-yl)amino)-7,8-dihydro-1,6-naphthyridin-6(5H)-yl)-2,8-
dimethyl-4H-pyrimido[1,2-b]pyridazin-4-one
7-(3-((5-fluoro-2-methylpyridin-4-yl)amino)-7,8-dihydro-1,6-naphthyridin-6(5H)-yl-
5,5,7,7-d4)-8-methyl-4H-pyrimido[1,2-b]pyridazin-4-one
8-methyl-7-(3-((2-methylpyridin-3-yl)amino)-7,8-dihydro-1,6-naphthyridin-6(5H)-yl-
5,5,7,7-d4)-4H-pyrimido[1,2-b]pyridazin-4-one
7-(3-((3-fluoropyridin-4-yl)amino)-7,8-dihydro-1,6-naphthyridin-6(5H)-yl-5,5,7,7-d4)-8-
methyl-4H-pyrimido[1,2-b]pyridazin-4-one
8-methyl-7-(3-((3-methylpyridin-4-yl)amino)-7,8-dihydro-1,6-naphthyridin-6(5H)-yl-
5,5,7,7-d4)-4H-pyrimido[1,2-b]pyridazin-4-one
7-(3-((2-fluorophenyl)amino)-7,8-dihydro-1,6-naphthyridin-6(5H)-yl-5,5,7,7-d4)-8-methyl-
4H-pyrimido[1,2-b]pyridazin-4-one

or a pharmaceutically acceptable salt thereof.

E21. A pharmaceutical composition comprising the compound of any of E1-E20, or a pharmaceutically acceptable salt thereof, and a pharmaceutically acceptable carrier.

E22. A method for treating a neurological and/or psychiatric disorder associated with muscarinic acetylcholine receptor dysfunction in a mammal, comprising administering to the mammal a therapeutically effective amount of the compound of any of E1-E20, or pharmaceutically acceptable salt thereof, or the pharmaceutical composition of claim 21.

E23. The method of E22, wherein the disorder is associated with a mAChR M₄ dysfunction.

E24. The method of E22 or E23, wherein the disorder is a neurological and/or psychiatric disorder associated with mAChR M₄ dysfunction.

E25. The method of any of E22-E24, wherein the disorder is selected from the group consisting of Alzheimer's disease, schizophrenia, a sleep disorder, a pain disorder, and a cognitive disorder.

E26. The method of E25, wherein the disorder is Alzheimer's disease.

E27. The method of any of E22-E24, wherein the disorder is selected from the group consisting of psychosis, schizophrenia, conduct disorder, disruptive behavior disorder, bipolar disorder, psychotic episodes of anxiety, anxiety associated with psychosis, psychotic mood disorders such as severe major depressive disorder; mood disorders associated with psychotic disorders, acute mania, depression associated with bipolar disorder, mood disorders associated with schizophrenia, behavioral manifestations of mental retardation, autistic disorder, movement disorders, Tourette's syndrome, akinetic-rigid syndrome, movement disorders associated with Parkinson's disease, tardive dyskinesia, drug induced and neurodegeneration based dyskinesias, attention deficit hyperactivity disorder, cognitive disorders, dementias, and memory disorders.

E28. A kit comprising the compound of any of E1-E20, or a pharmaceutically acceptable salt thereof, and one or more of: (a) at least one agent known to increase mAChR M₄ activity; (b) at least one agent known to decrease mAChR M₄ activity; (c) at least one agent known to treat a disorder associated with cholinergic activity; (d) instructions for treating a disorder associated with cholinergic activity; (e) instructions for treating a disorder associated with mAChR M₄ receptor activity; and (f) instructions for administering the compound in connection with cognitive or behavioral therapy.

E29. The compound of any of E1-E20, or a pharmaceutically acceptable salt thereof, or the pharmaceutical composition of E21, for use in the treatment of a neurological and/or psychiatric disorder associated with muscarinic acetylcholine receptor dysfunction in a mammal.

E30. The use of the compound of any of E1-E20, or a pharmaceutically acceptable salt thereof, or the pharmaceutical composition of E21 for the preparation of a medicament for the treatment of a neurological and/or psychiatric disorder associated with muscarinic acetylcholine receptor dysfunction in a mammal.

The compound may exist as a stereoisomer wherein asymmetric or chiral centers are present. The stereoisomer is “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The disclosure contemplates various stereoisomers and mixtures thereof and these are specifically included within the scope of this invention. Stereoisomers include enantiomers and diastereomers, and mixtures of enantiomers or diastereomers. Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry,” 5th edition (1989), Longman Scientific & Technical, Essex CM20 2JE, England, or (2) direct separation of the mixture of optical enantiomers on chiral chromatographic columns, or (3) fractional recrystallization methods.

It should be understood that the compound may possess tautomeric forms, as well as geometric isomers, and that these also constitute embodiments of the disclosure.

In the compounds of formula (I), and any subformulas, any “hydrogen” or “H,” whether explicitly recited or implicit in the structure, encompasses hydrogen isotopes ¹H (protium) and ²H (deuterium).

The present disclosure also includes an isotopically-labeled compound, which is identical to those recited in formula (I), but for the fact that one or more atoms are replaced by an atom having an atomic mass or mass number different from the atomic mass or mass number usually found in nature. Examples of isotopes suitable for inclusion in the compounds of the invention are hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ¹⁷O, ³¹P, ³²P, ³⁵S, ¹⁸F, and ³⁶Cl, respectively. Substitution with heavier isotopes such as deuterium, i.e. ²H, can afford certain therapeutic advantages resulting from greater metabolic stability, for example increased in vivo half-life or reduced dosage requirements and, hence, may be preferred in some circumstances. The compound may incorporate positron-emitting isotopes for medical imaging and positron-emitting tomography (PET) studies for determining the distribution of receptors. Suitable positron-emitting isotopes that can be incorporated in compounds of formula (I) are ¹¹C, ¹³N, ¹⁵O, and ¹⁸F.

Isotopically-enriched forms of compounds of formula (I), or any subformulas, may generally be prepared by conventional techniques known to those skilled in the art or by processes analogous to those described in the accompanying Examples using an appropriate isotopically-enriched reagent in place of a non-isotopically-enriched reagent. The extent of isotopic enrichment can be characterized as a percent incorporation of a particular isotope at an isotopically-labeled atom (e.g., % deuterium incorporation at a deuterium label).

In some embodiments, R¹ is deuterium. In some embodiments, R² is deuterium. In some embodiments, R⁵ is deuterium.

a. Pharmaceutically Acceptable Salts

The disclosed compounds may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and driedunder reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

b. General Synthesis

Compounds of formula (I) may be prepared by synthetic processes or by metabolic processes. Preparation of the compounds by metabolic processes includes those occurring in the human or animal body (in vivo) or processes occurring in vitro. Abbreviations used in the schemes that follow include the following: DCE is 1,2-dichloroethane; DCM is dichloromethane; (4,4′-dtbbpy)NiCl₂ is 4,4′-bis(1,1-dimethylethyl)-2,2′-bipyridine]nickel (II) dichloride; DMF is N,N-dimethylformamide; DMP or Dess-Martin periodinane is 1,1,1-tris(acetyloxy)-1,1-dihydro-1,2-benziodoxol-3-(1H)-one; Dowtherm™ A is a eutectic mixture of 26.5% diphenyl+73.5% diphenyl oxide; DtBAD is di-tert-butyl azodicarboxylate; Et is ethyl; HPLC is high-performance liquid chromatography; Hunig's base is N,N-diisopropylethylamine; Ir[dF(CF₃)ppy]₂(dtbpy))PF₆ is [4,4′-bis(1,1-dimethylethyl)-2,2′-bipyridine-N1,N1′]bis[3,5-difluoro-2-[5-(trifluoromethyl)-2-pyridinyl-N]phenyl-C]Iridium(III) hexafluorophosphate; LAH is lithium aluminum hydride; mCPBA is meta-chloroperoxy benzoic acid; Me is methyl; MeOH is methanol; MW is microwave (referring to a microwave reactor); Pd₂(dba)₃ is tris(dibenzylideneacetone)dipalladium(0); Pd(dppf)Cl₂ is [1,1′-bis(diphenylphosphino)ferrocene]dichloropalladium(II); Pd(OAc)₂ is palladium(II) acetate; PPA is polyphosphoric acid; Rxn is reaction; TBAC is tetrabutylammonium chloride; t-BuXPhos is 2-di-tert-butylphosphino-2′,4′,6′-triisopropylbiphenyl; and THF is tetrahydrofuran.

Compounds of formula (I) may be synthesized as shown in Schemes 1-12.

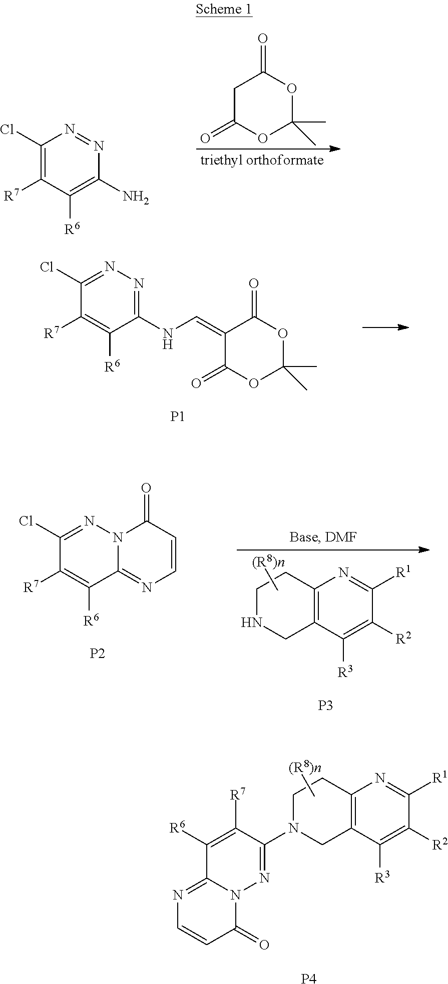

As shown in Scheme 1, reaction of analogues of 6-chloropyridazin-3-amine with triethyl orthoformate and isopropylidene malonate may provide compounds of type P1, which may be cyclized to provide compounds of type P2. Reaction of compounds P2 with 5,6,7,8-tetrahydronaphthyridines of formula P3 in a solvent such as dimethylformamide (DMF) in the presence of a base (e.g., Hunig's base) may provide compounds P4 (e.g., R² is H or halo).

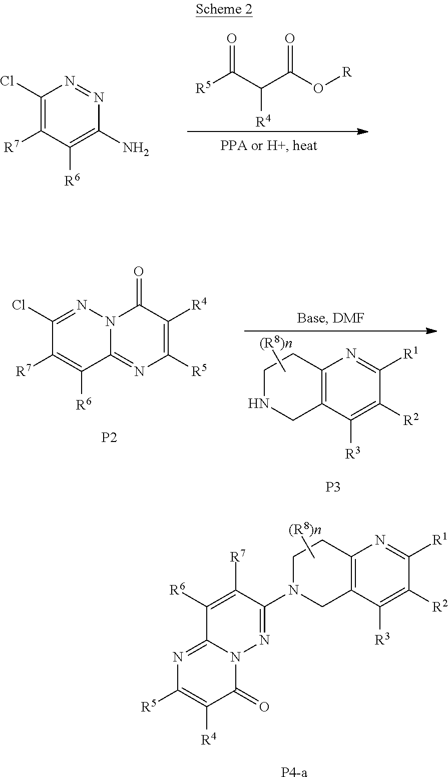

As shown in Scheme 2, reaction of analogues of 6-chloropyridazin-3-amine with beta-ketoacids may be cyclized using an acid (e.g., PPA) to provide compounds of type P2. Reaction of compounds P2 with 5,6,7,8-tetrahydronaphthyridines of formula P3 in a solvent such as dimethylformamide (DMF) in the presence of a base (e.g., Hunig's base) may provide compounds P4 (e.g., R² is H or halo).

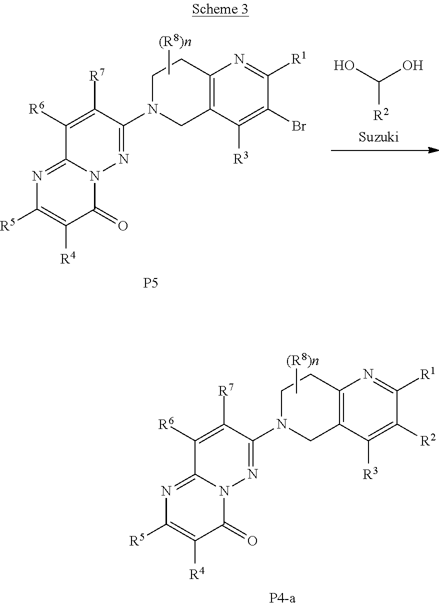

As shown in Scheme 3, intermediates of type P5 may be coupled with a boronic acid or ester under Suzuki coupling conditions, generally known in the art, to provide compounds of formula P4-a, wherein R² is alkyl or G², and G² is an optionally substituted aryl or heteroaryl ring system as defined herein. Coupling reactions may be conducted with a palladium catalyst, such as Pd(dppf)Cl₂, and a base (e.g., K₂CO₃, Cs₂CO₃) in a solvent mixture of organic solvent, such as DMF or dioxane, and water with heating to about 70-90° C. The reaction may be facilitated with microwave irradiation.

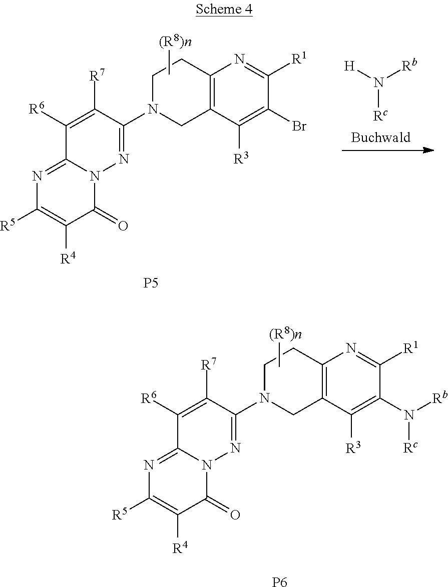

As shown in Scheme 4, intermediates of type P5 may be coupled with an amine under Buchwald coupling conditions, generally known in the art, to provide products of type P6, wherein R^{b} and R^{c} are as defined herein. For example, the reaction may be conducted with a palladium catalyst, such as Pd₂(dba)₃, in the presence of a base (e.g., Cs₂CO₃) and a ligand, such as Xantphos (4,5-bis(diphenylphosphino)-9,9-dimethylxanthene), in a solvent, such as dioxane with heating up to around 100° C. The synthetic route depicted in Scheme 4 may likewise be applied to the synthesis of compounds of formula (I) wherein R² is G² and G² is a heterocycle attached at a nitrogen atom (e.g., morpholine).

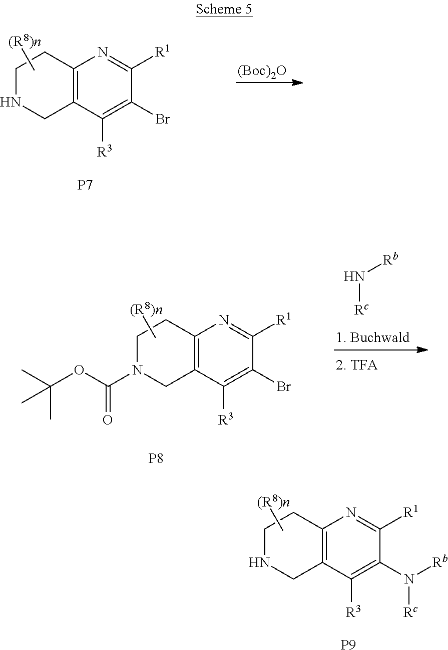

As shown in Scheme 5, intermediates of type P7 may be protected with di-t-butyl carbonate under Boc-protection conditions, generally known in the art, to provide Boc-protected intermediates of type P8. Intermediates of type P8 may be coupled with an amine under Buchwald coupling conditions, generally known in the art, followed by deprotection to provide products of type P9.

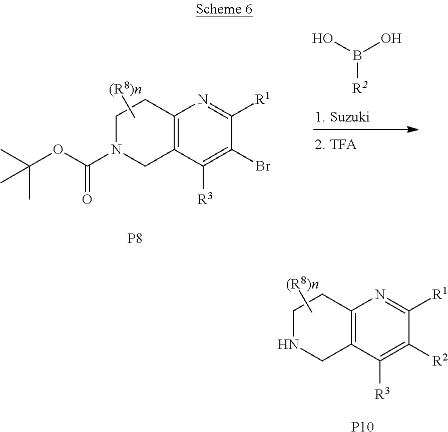

Scheme 6 illustrates a general route to prepare intermediates of the formula P10. Intermediates of the type P8 may be coupled with a boronic acid or ester under Suzuki coupling conditions, generally known in the art, followed by deprotection to provide compounds of formula P10, wherein R² is alkyl or G², and G² is an optionally substituted aryl or heteroaryl ring system as defined herein. Coupling reactions may be conducted with a palladium catalyst, such as Pd(dppf)Cl₂, and a base (e.g., K₂CO₃, Cs₂CO₃) in a solvent mixture of organic solvent, such as DMF or dioxane, and water with heating to about 70-90° C. The reaction may be facilitated with microwave irradiation.

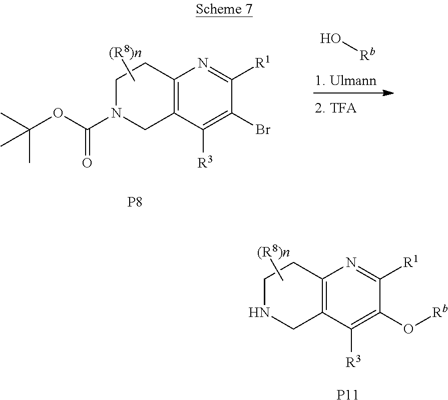

As shown in Scheme 7, compounds of formula P8 may be reacted with alcohols under Ullmann conditions, generally known in the art, followed by deprotection with an acid (e.g., TFA) to provide intermediates of type P11. Suitable Ullmann conditions for coupling with a phenol include the use of a base (e.g., Cs₂CO₃), 2,2,6,6-tetramethylheptane-3,5-dione, and a copper salt (e.g., copper (I) iodide) with heating in a solvent, such as NMP, up to around 100-120° C.

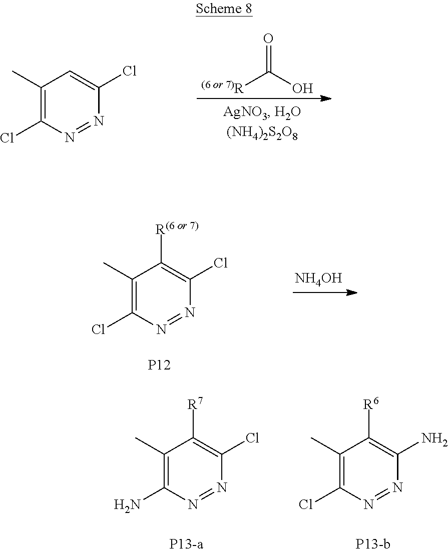

As shown in Scheme 8, analogues of 6-chloropyridazin-3-amine P13-a and P13-b may be prepared by a radical based addition of an appropriate carboxylic acid to provide intermediate P12, which may be transformed to the desired intermediates P13-a and P13-b under suitable conditions (e.g., using NH₄OH).

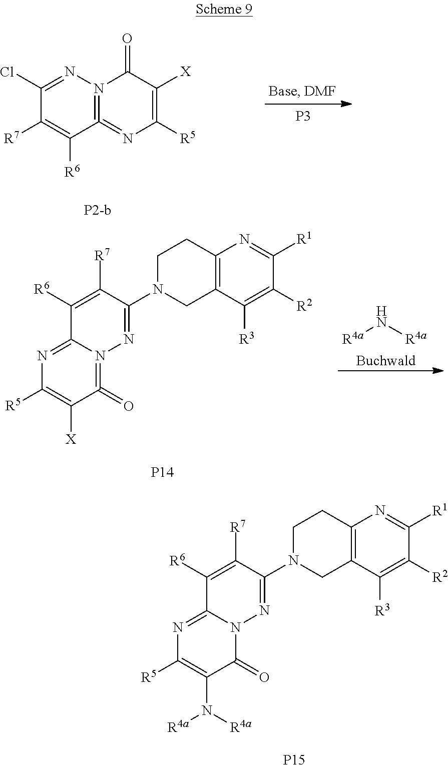

As shown in Scheme 9, reaction of compounds P2-b (e.g., where X is a halogen) with 5,6,7,8-tetrahydronaphthyridines of formula P3 in a solvent such as Dimethylformamide (DMF) in the presence of a base (e.g., Hunig's base) may provide compounds P14. Intermediate of type P14 may be coupled with an amine under Buchwald coupling conditions, generally known in the art, to provide products of type P15, wherein R^{4a} is as defined herein. For example, the reaction may be conducted with a palladium catalyst, such as Pd₂(dba)₃, in the presence of a base (e.g., Cs₂CO₃) and a ligand, such as Xantphos (4,5-bis(diphenylphosphino)-9,9-dimethylxanthene), in a solvent such as dioxane with heating up to around 100° C. The synthetic route depicted in Scheme 9 may likewise be applied to the synthesis of compounds of formula (I) wherein R⁴ is G⁴, and G⁴ is a heterocycle attached at a nitrogen atom (e.g., morpholine).

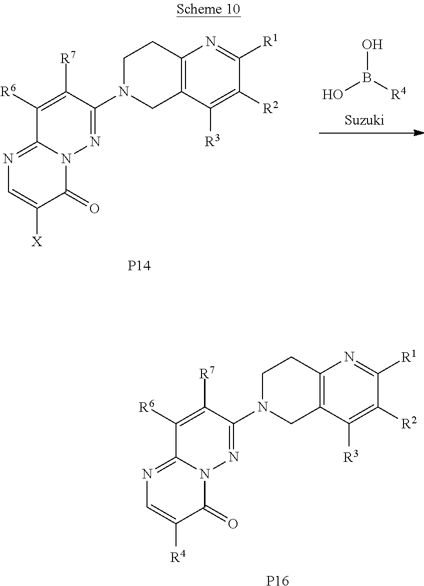

Scheme 10 illustrates a general route to prepare intermediates of the formula P16. Intermediates of the type P14 may be coupled with a boronic acid or ester under Suzuki coupling conditions, generally known in the art, wherein R⁴ is alkyl or G⁴, and G⁴ is an optionally substituted aryl or heteroaryl ring system as defined herein. Coupling reactions may be conducted with a palladium catalyst such as Pd(dppf)Cl₂ and a base (e.g., K₂CO₃, Cs₂CO₃) in a solvent mixture of organic solvent and water such as DMF or dioxane and water with heating to about 70-90° C. The reaction may be facilitated with microwave irradiation.

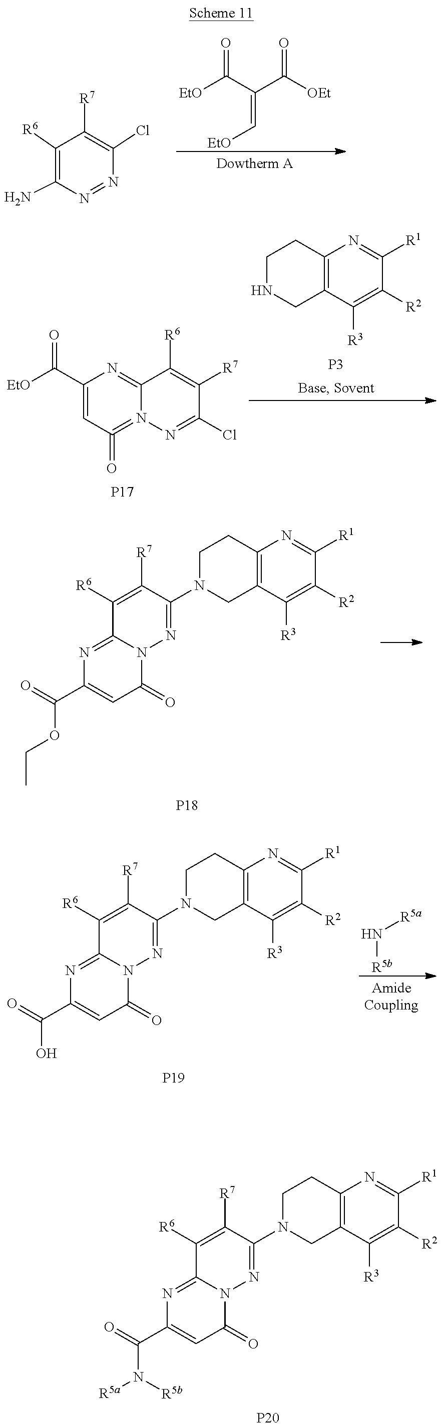

Scheme 11 shows a general route to compounds of formula P20. The reaction of analogues of 6-chloropyridazin-3-amine with diethyl ethoxymethylenemalonatein a solvent (e.g., Dowtherm™ A) with heating (e.g., 180-220° C.) may provide intermediates of type P17. The reaction of compounds P17 with 5,6,7,8-tetrahy dronaphthyridines of formula P3 in a solvent, such as tert-butanol (t-BuOH), in the presence of a base (e.g., Hunig's base) may provide compounds P18. After intermediate P18 is subjected to standard hydrolysis conditions, such as NaOH, the resulting carboxylic acid P19 may undergo amide formation to provide compounds of the formula P20.

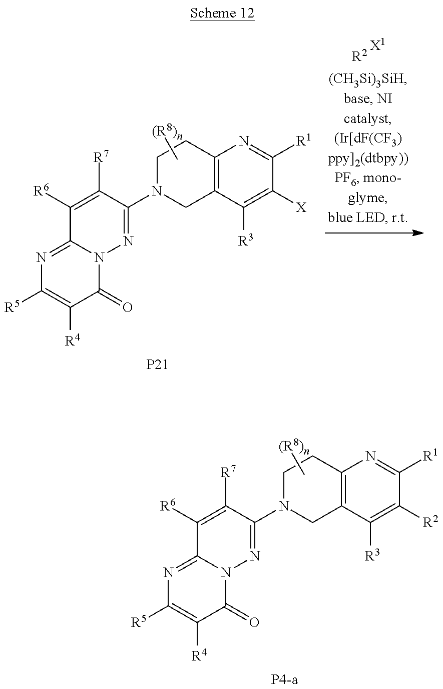

Scheme 12 illustrates an alternative route to compounds of the formula P4-a. Intermediate P21 (e.g., where X=halogen) in the presence of alkyl halides or saturated heterocyclic halides (e.g., R²—X¹), base (e.g., LiOH), tris(trimethylsilyl)silane, an Ir catalyst (e.g., (Ir[dF(CF₃)ppy]₂(dtbpy))PF₆), and a Ni catalyst (e.g. (4,4′-dtbbpy)NiCl₂) under a blue LED light may undergo a transformation to form P4-a.

Suitable boronic acids/esters, amines, and alcohols for coupling reactions described herein may be readily obtained from commerical sources or prepared by standard methods well know to those skilled in the art.

The compounds and intermediates may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry,” 5th edition (1989), by Furniss, Hannaford, Smith, and Tatchell, pub. Longman Scientific & Technical, Essex CM20 2JE, England.

A disclosed compound may have at least one basic nitrogen whereby the compound can be treated with an acid to form a desired salt. For example, a compound may be reacted with an acid at or above room temperature to provide the desired salt, which is deposited, and collected by filtration after cooling. Examples of acids suitable for the reaction include, but are not limited to tartaric acid, lactic acid, succinic acid, as well as mandelic, atrolactic, methanesulfonic, ethanesulfonic, toluenesulfonic, naphthalenesulfonic, benzenesulfonic, carbonic, fumaric, maleic, gluconic, acetic, propionic, salicylic, hydrochloric, hydrobromic, phosphoric, sulfuric, citric, hydroxybutyric, camphorsulfonic, malic, phenylacetic, aspartic, or glutamic acid, and the like.

Reaction conditions and reaction times for each individual step can vary depending on the particular reactants employed and substituents present in the reactants used. Specific procedures are provided in the Examples section. Reactions can be worked up in the conventional manner, e.g. by eliminating the solvent from the residue and further purified according to methodologies generally known in the art such as, but not limited to, crystallization, distillation, extraction, trituration and chromatography. Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which can be found in PGM Wuts and TW Greene, in Greene's book titled Protective Groups in Organic Synthesis (4^{th} ed.), John Wiley & Sons, NY (2006), which is incorporated herein by reference in its entirety. Synthesis of the compounds of the invention can be accomplished by methods analogous to those described in the synthetic schemes described hereinabove and in specific examples.

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound is required, it can be obtained by carrying out one of the above procedures using a pure geometric isomer as a starting material, or by resolution of a mixture of the geometric isomers of the compound or intermediates using a standard procedure such as chromatographic separation.

It can be appreciated that the synthetic schemes and specific examples as described are illustrative and are not to be read as limiting the scope of the invention as it is defined in the appended claims. All alternatives, modifications, and equivalents of the synthetic methods and specific examples are included within the scope of the claims.

c. Muscarinic Acetylcholine Receptor M₄ Activity

In some embodiments, the disclosed compounds potentiate the agonist response (e.g., acetylcholine) of mAChR M₄. In some embodiments, the disclosed compounds increase mAChR M₄ response to non-maximal concentrations of agonist in the presence of compound compared to the response to agonistin the absence of compound. The potentiation of mAChRM₄ activity can be demonstrated by methodology known in the art. For example, activation of mAChR M₄ activity can be determined by measurement of calcium flux in response to agonist, e.g. acetylcholine, in cells loaded with a Ca²⁺-sensitive fluorescent dye (e.g., Fluo-4) and co-expression of a chimeric or promiscuous G protein. In some embodiments, the calcium flux was measured as an increase in fluorescent static ratio. In some embodiments, positive allosteric modulator activity was analyzed as a concentration-dependent increase in the EC₂₀ acetylcholine response (i.e. the response of mAChR M₄ at a concentration of acetylcholine that yields 20% of the maximal response).

In some embodiments, the disclosed compounds activate mAChR M₄ response as an increase in calcium fluorescence in mAChR M₄-transfected CHO-K1 cells in the presence of the compound, compared to the response of equivalent CHO-K1 cells in the absence of the compound. In some embodiments, a disclosed compound activates the mAChR M₄ response with an EC₅₀ of less than about 10 μM, less than about 5 μM, less than about 1 μM, less than about 500 nM, of less than about 100 nM, or less than about 50 nM. In some embodiments, the mAChR M₄-transfected CHO-K1 cells are transfected with human mAChR M₄ In some embodiments, the mAChRM₄-transfected CHO-K1 cells are transfected with ratmAChRM₄.

The disclosed compounds may exhibit positive allosteric modulation ofmAChRM₄ response to acetylcholine as an increase in response to non-maximal concentrations of acetylcholine in CHO-K1 cells transfected with a mAChR M₄ in the presence of the compound, compared to the response to acetylcholine in the absence of the compound. In some embodiments, the disclosed compounds exhibit positive allosteric modulation of the mAChR M₄ response to acetylcholine with an EC₅₀ of less than about 10 μM, less than about 5 μM, less than about 1 μM, less than about 500 nM, or less than about 100 nM. In some embodiments, the EC₅₀ for positive allosteric modulation is determined in CHO-K1 cells are transfected with a mAChR M₄. In some embodiments, the mAChR M₄ transfected human mAChR M₄. In some embodiments, the mAChR M₄ transfected rat mAChR M₄.

The disclosed compounds may activate mAChR M₄ response in mAChR M₄-transfected CHO-K1 cells with an EC₅₀ less than the EC₅₀ for one or more of mAChR M₁, M₂, M₃ orM₅-transfected CHO-K1 cells. That is, a disclosed compound can have selectivity for the mAChR M₄ receptor vis-à-vis one or more of the mAChR M₁, M₂, M₃ or M₅ receptors. For example, in some embodiments, a disclosed compound can activate mAChR M₄ response with an EC₅₀ of about 5-fold less, about 10-fold less, about 20-fold less, about 30-fold less, about 50-fold less, about 100-fold less, about 200-fold less, about 300-fold less, about 400-fold less, or greater than about 500-fold less than that for mAChR M₁. In some embodiments, a disclosed compound can activate mAChR M₄ response with an EC₅₀ of about 5-fold less, about 10-fold less, about 20-fold less, about 30-fold less, about 50-fold less, about 100-fold less, about 200-fold less, about 300-fold less, about 400-fold less, or greater than about 500-fold less than that for mAChR M₂. In some embodiments, a disclosed compound can activate mAChR M₄ response with an EC₅₀ of about 5-fold less, about 10-foldless, about 20-fold less, about 30-fold less, about 50-fold less, about 100-fold less, about 200-fold less, about 300-fold less, about 400-fold less, or greater than about 500-fold less than that for mAChR M₃. In some embodiments, a disclosed compound can activate mAChR M₄ response with an EC₅₀ of about 5-fold less, about 10-fold less, about 20-fold less, about 30-fold less, about 50-fold less, about 100-fold less, about 200-fold less, about 300-fold less, about 400-fold less, or greater than about 500-fold less than that for mAChR M₅. In some embodiments, a disclosed compound can activate mAChR M₄ response with an EC₅₀ of 5-fold less, about 10-fold less, about 20-fold less, about 30-fold less than that for the M₂-M₅ receptors, of about 50-fold less, about 100-fold less, about 200-fold less, about 300-fold less, about 400-fold less, or greater than about 500-fold less than that for the mAChR M₁, M₂, M₃, or M₅ receptors.

The disclosed compounds may activate mAChR M₄ response in M₄-transfected CHO-K1 cells with an EC₅₀ of less than about 10 μM and exhibits a selectivity for the M₄ receptor vis-à-vis one or more of the mAChR M₁, M₂, M₃, or M₅ receptors. For example, in some embodiments, the compound can have an EC₅₀ of less than about 10 μM, of less than about 5 μM, of less than about 1 μM, of less than about 500 nM, of less than about 100 nM, or of less than about 50 nM; and the compound can also activate mAChR M₄ response with an EC₅₀ of about 5-fold less, 10-fold less, 20-fold less, 30-fold less, 50-fold less, 100-fold less, 200-fold less, 300-fold less, 400-fold less, or greater than about 500-fold less than that for mAChR M₁. In some embodiments, the compound can have an EC₅₀ of less than about 10 μM, of less than about 5 μM, of less than about 1 μM, of less than about 500 nM, of less than about 100 nM, or of less than about 50 nM; and the compound can also activate mAChR M₄ response with an EC₅₀ of about 5-fold less, about 10-fold less, about 20-fold less, about 30-fold less, about 50-fold less, about 100-fold less, about 200-fold less, about 300-fold less, about 400-fold less, or greater than about 500-fold less than that for mAChRM₂. In some embodiments, the compound can have an EC₅₀ of less than about 10 μM, of less than about 5 μM, of less than about 1 μM, of less than about 500 nM, of less than about 100 nM, or of less than about 50 nM; and the compound can also activate mAChR M₄ response with an EC₅₀ of about 5-fold less, about 10-fold less, about 20-fold less, about 30-fold less, about 50-fold less, about 100-fold less, about 200-fold less, about 300-fold less, about 400-fold less, or greater than about 500-fold less than that for mAChR M₃. In some embodiments, the compound can have an EC₅₀ of less than about 10 μM, of less than about 5 μM, of less than about 1 μM, of less than about 500 nM, of less than about 100 nM, or of less than about 50 nM; and the compound can also activate mAChR M₄ response with an EC₅₀ of about 5-fold less, about 10-fold less, about 20-fold less, about 30-fold less, about 50-fold less, about 100-fold less, about 200-fold less, about 300-fold less, about 400-fold less, or greater than about 500-fold less than that for mAChR M₅. In some embodiments, the compound can have an EC₅₀ of less than about 10 μM, of less than about 5 μM, of less than about 1 μM, of less than about 500 nM, of less than about 100 nM, or of less than about 50 nM; and the compound can also activate mAChRM₄ response with EC₅₀ of 5-fold less, about 10-fold less, about 20-fold less, about 30-fold less than that for the M₂-M₅ receptors, of about 50-fold less, about 100-fold less, about 200-fold less, about 300-fold less, about 400-fold less, M₂, M₃, or M₅ receptors, or greater than about 500-fold less than that for the mAChR M₁, M₂, M₃, or M₅ receptors.

In vivo efficacy for disclosed compounds can be measured in a number of preclinical rat behavioral models where known, clinically useful antipsychotics display similar positive responses. For example, disclosed compounds may reverse amphetamine-induced hyperlocomotion in male Sprague-Dawley rats at doses ranging from 1 to 100 mg/kg p.o.

### 3. PHARMACEUTICAL COMPOSITIONS AND FORMULATIONS

The disclosed compounds may be incorporated into pharmaceutical compositions suitable for administration to a subject (such as a patient, which may be a human or non-human). The disclosed compounds may also be provided as formulations, such as spray-dried dispersion formulations.

The pharmaceutical compositions and formulations may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the invention (e.g., a compound of formula (I)) are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

For example, a therapeutically effective amount of a compound of formula (I), may be about 1 mg/kg to about 1000 mg/kg, about 5 mg/kg to about 950 mg/kg, about 10 mg/kg to about 900 mg/kg, about 15 mg/kg to about 850 mg/kg, about 20 mg/kg to about 800 mg/kg, about 25 mg/kg to about 750 mg/kg, about 30 mg/kg to about 700 mg/kg, about 35 mg/kg to about 650 mg/kg, about 40 mg/kg to about 600 mg/kg, about 45 mg/kg to about 550 mg/kg, about 50 mg/kg to about 500 mg/kg, about 55 mg/kg to about 450 mg/kg, about 60 mg/kg to about 400 mg/kg, about 65 mg/kg to about 350 mg/kg, about 70 mg/kg to about 300 mg/kg, about 75 mg/kg to about 250 mg/kg, about 80 mg/kg to about 200 mg/kg, about 85 mg/kg to about 150 mg/kg, and about 90 mg/kg to about 100 mg/kg.

The pharmaceutical compositions and formulations may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, the compounds and their pharmaceutically acceptable salts may be formulated for administration by, for example, solid dosing, eye drop, in a topical oil-based formulation, injection, inhalation (either through the mouth or the nose), implants, or oral, buccal, parenteral, or rectal administration. Techniques and formulations may generally be found in “Remington's Pharmaceutical Sciences,” (Meade Publishing Co., Easton, Pa.). Therapeutic compositions must typically be sterile and stable under the conditions of manufacture and storage.

The route by which the disclosed compounds are administered and the form of the composition will dictate the type of carrier to be used. The composition may be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, oriontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmellose, crospovidone, sodium carboxymethyl starch, sodium starch glycolate, clays, and ion exchange resins. The amount of disintegrant(s) in a systemic or topical composition is typically about 0.1 to about 10%.

Suitable colorants include a colorant such as an FD&C dye. When used, the amount of colorant in a systemic or topical composition is typically about 0.005 to about 0.1%.

Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s) in a systemic or topical composition is typically about 0.001 to about 1%.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5%.

Suitable preservatives include benzalkonium chloride, methyl paraben and sodium benzoate. The amount of preservative(s) in a systemic or topical composition is typically about 0.01 to about 5%.

Suitable glidants include silicon dioxide. The amount of glidant(s) in a systemic or topical composition is typically about 1 to about 5%.

Suitable solvents include water, isotonic saline, ethyl oleate, glycerine, hydroxylated castor oils, alcohols such as ethanol, and phosphate buffer solutions. The amount of solvent(s) in a systemic or topical composition is typically from about 0 to about 100%.

Suitable suspending agents include AVICEL RC-591 (from FMC Corporation of Philadelphia, PA) and sodium alginate. The amount of suspending agent(s) in a systemic or topical composition is typically about 1 to about 8%.

Suitable surfactants include lecithin, Polysorbate 80, and sodium lauryl sulfate, and the TWEENS from Atlas Powder Company of Wilmington, Delaware. Suitable surfactants include those disclosed in the C.T.F.A. Cosmetic Ingredient Handbook, 1992, pp. 587-592; Remington's Pharmaceutical Sciences, 15th Ed. 1975, pp. 335-337; andMcCutcheon's Volume 1, Emulsifiers & Detergents, 1994, North American Edition, pp. 236-239. The amount of surfactant(s) in the systemic or topical composition is typically about 0.1% to about 5%.

Although the amounts of components in the systemic compositions may vary depending on the type of systemic composition prepared, in general, systemic compositions include 0.01% to 50% of an active compound (e.g., a compound of formula (I)) and 50% to 99.99% of one or more carriers. Compositions for parenteral administration typically include 0.1% to 10% of actives and 90% to 99.9% of a carrier including a diluent and a solvent.

Compositions for oral administration can have various dosage forms. For example, solid forms include tablets, capsules, granules, and bulk powders. These oral dosage forms include a safe and effective amount, usually at least about 5%, and more particularly from about 25% to about 50% of actives. The oral dosage compositions include about 50% to about 95% of carriers, and more particularly, from about 50% to about 75%.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose and cellulose. Specific binders include starch, gelatin, and sucrose. Specific disintegrants include alginic acid and croscarmellose. Specific lubricants include magnesium stearate, stearic acid, and talc. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active compound (e.g., a compound of formula (I)), and a carrier including one or more diluents disclosed above in a capsule comprising gelatin. Granules typically comprise a disclosed compound, and preferably glidants such as silicon dioxide to improve flow characteristics. Implants can be of the biodegradable or the non-biodegradable type.

The selection of ingredients in the carrier for oral compositions depends on secondary considerations like taste, cost, and shelf stability, which are not critical for the purposes of this invention.

Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGIT® coatings (available from Evonik Industries of Essen, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of the subject compounds include sublingual, buccal and nasal dosage forms. Such compositions typically include one or more of soluble filler substances such as diluents including sucrose, sorbitol and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

The disclosed compounds can be topically administered. Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions include: a disclosed compound (e.g., a compound of formula (I)), and a carrier. The carrier of the topical composition preferably aids penetration of the compounds into the skin. The carrier may further include one or more optional components.

The amount of the carrier employed in conjunction with a disclosed compound is sufficient to provide a practical quantity of composition for administration per unit dose of the compound. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and poly dimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%.

The amount of thickener(s) in a topical composition is typically about 0% to about 95%.

Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%.

The amount of fragrance in a topical composition is typically about 0% to about 0.5%, particularly, about 0.001% to about 0.1%.

Suitable pH adjusting additives include HCl or NaOH in amounts sufficient to adjust the pH of a topical pharmaceutical composition.

The pharmaceutical composition or formulation may exhibit positive allosteric modulation of mAChR M₄ with an EC₅₀ of less than about 10 μM, less than about 5 μM, less than about 1 μM, less than about 500 nM, or less than about 100 nM. The pharmaceutical composition or formulation may exhibit positive allosteric modulation of mAChR M4 with an EC₅₀ of between about 10 μM and about 1 nM, about 1 μM and about 1 nM, about 100 nM and about 1 nM, or between about 10 nMand about 1 nM.

a. Spray-Dried Dispersion Formulations

The disclosed compounds may be formulated as a spray-dried dispersion (SDD). An SDD is a single-phase, amorphous molecular dispersion of a drug in a polymer matrix. It is a solid solution with the compound molecularly “dissolved” in a solid matrix. SDDs are obtained by dissolving drug and a polymer in an organic solvent and then spray-drying the solution. The use of spray drying for pharmaceutical applications can result in amorphous dispersions with increased solubility of Biopharmaceutics Classification System (BCS) class II (high permeability, low solubility) and class IV (low permeability, low solubility) drugs. Formulation and process conditions are selected so that the solvent quickly evaporates from the droplets, thus allowing insufficient time for phase separation or crystallization. SDDs have demonstrated long-term stability and manufacturability. For example, shelf lives of more than 2 years have been demonstrated with SDDs. Advantages of SDDs include, but are not limited to, enhanced oral bioavailability of poorly water-soluble compounds, delivery using traditional solid dosage forms (e.g., tablets and capsules), a reproducible, controllable and scalable manufacturing process and broad applicability to structurally diverse insoluble compounds with a wide range of physical properties.

Thus, in one embodiment, the disclosure may provide a spray-dried dispersion formulation comprising a compound of formula (I).

### 4. METHODS OF USE

The disclosed compounds, pharmaceutical compositions and formulations may be used in methods for treatment of disorders, such as neurological and/or psychiatric disorders, associated with muscarinic acetylcholine receptor dysfunction. The disclosed compounds and pharmaceutical compositions may also be used in methods for the potentiation of muscarinic acetylcholine receptor activity in a mammal, and in methods for enhancing cognition in a mammal. The methods further include cotherapeutic methods for improving treatment outcomes in the context of cognitive or behavioral therapy. In the methods ofuse described herein, additional therapeutic agent(s) may be administered simultaneously or sequentially with the disclosed compounds and compositions.

a. Treating Disorders

The disclosed compounds, pharmaceutical compositions and formulations may be used for treating disorders, or used in methods for treatment of disorders, such as neurological and/or psychiatric disorders, associated with muscarinic acetylcholine receptor dysfunction. The methods of treatment may comprise administering to a subject in need of such treatment a therapeutically effective amount of the compound of formula (I), or a pharmaceutical composition comprising a therapeutically effective amount of a compound of formula (I).

In some embodiments, the disclosure provides a method for enhancing cognition in a mammal comprising the step of administering to the mammal a therapeutically effective amount of the compound of formula (I), or a pharmaceutical composition comprising a therapeutically effective amount of a compound of formula (I).

The compounds and compositions disclosed herein may be useful for treating, preventing, ameliorating, controlling or reducing the risk of a variety of disorders associated with selective mAChR M₄ receptor activation. For example, a treatment can include selective mAChR M₄ receptor activation to an extent effective to affect cholinergic activity. A disorder can be associated with cholinergic activity, for example cholinergic hypofunction. Thus, provided is a method of treating or preventing a disorder in a subject comprising the step of administering to the subject at least one disclosed compound or at least one disclosed pharmaceutical composition, in an amount effective to treat the disorder in the subject.

Also provided is a method for the treatment of one or more disorders associated with mAChR M₄ receptor activity in a subject comprising the step of administering to the subject a therapeutically effective amount of the compound of formula (I), or a pharmaceutically acceptable salt thereof, or a pharmaceutical composition comprising a therapeutically effective amount of a compound of formula (I), or a pharmaceutically acceptable salt thereof.

In some embodiments, the disclosure provides a compound of formula (I), or a pharmaceutically acceptable salt thereof, for use in a method for the treatment of a disorder associated with the mAChR M₄ receptor. In some embodiments, the disclosure provides a pharmaceutical composition comprising a compound of formula (I), or a pharmaceutically acceptable salt thereof, for use in a method for the treatment of a disorder associated with the mAChR M₄ receptor.

In some embodiments, the disclosure provides a compound of formula (I), or a pharmaceutically acceptable salt thereof, for use in the manufacture of a medicament for the treatment of a disorder associated with the mAChR M₄ receptor.

In some embodiments, the disclosure provides a method for the treatment of a disorder associated with muscarinic acetylcholine receptor dysfunction in a mammal, comprising the step of administering to the mammal an effective amount of at least one disclosed compound or a pharmaceutically acceptable salt thereof, or a pharmaceutical composition comprising at least one disclosed compound or pharmaceutically acceptable salt thereof.

In some embodiments, the disclosure provides a compound of formula (I), or a pharmaceutically acceptable salt thereof, for use in a method for the treatment of a disorder associated with muscarinic acetylcholine receptor dysfunction in a mammal.

In some embodiments, the disclosure provides a pharmaceutical composition comprising a compound of formula (I), or a pharmaceutically acceptable salt thereof, for use in a method for the treatment of a disorder associated with muscarinic acetylcholine receptor dysfunction in a mammal.

In some embodiments, the disclosure provides a compound of formula (I), or a pharmaceutically acceptable salt thereof, for use in the manufacture of a medicament for the treatment of a disorder associated with muscarinic acetylcholine receptor dysfunction in a mammal.

In some embodiments, the disclosed compounds and compositions have utility in treating a variety of neurological, psychiatric and cognitive disorders associated with the mAChR M₄ receptor, including one or more of the following conditions or diseases: schizophrenia, psychotic disorder NOS, brief psychotic disorder, schizophreniform disorder, schizoaffective disorder, delusional disorder, shared psychotic disorder, catastrophic schizophrenia, postpartum psychosis, psychotic depression, psychotic break, tardive psychosis, myxedematous psychosis, occupational psychosis, menstrual psychosis, secondary psychotic disorder, bipolar I disorder with psychotic features, and substance-induced psychotic disorder. In some embodiments, the psychotic disorder is a psychosis associated with an illness selected from major depressive disorder, affective disorder, bipolar disorder, electrolyte disorder, Alzheimer's disease, neurological disorder, hypoglycemia, AIDS, lupus, and post-traumatic stress disorder.

In some embodiments, the disclosure provides a compound of formula (I), or a pharmaceutically acceptable salt thereof, for use in a method for the treatment of a neurological, psychiatric, or cognitive disorder associated with the mAChR M₄ receptor, in particular, the disorders described herein. In some embodiments, the disclosure provides a pharmaceutical composition comprising a compound of formula (I), or a pharmaceutically acceptable salt thereof, for use in a method for the treatment of a neurological, psychiatric, or cognitive disorder associated with the mAChR M₄ receptor, in particular, the disorders described herein. In some embodiments, the disclosure provides a compound of formula (I), or a pharmaceutically acceptable salt thereof, for use in the manufacture of a medicament for the treatment of a neurological, psychiatric, or cognitive disorder associated with the mAChR M₄ receptor, in particular, the disorders described herein.

In some embodiments, the disorder is a neurological disorder selected from brain tumor, dementia with Lewy bodies, multiple sclerosis, sarcoidosis, Lyme disease, syphilis, Alzheimer's disease, Parkinson's disease, and anti-NMDA receptor encephalitis.

In some embodiments, the disorder is a psychotic disorder selected from schizophrenia, brief psychotic disorder, schizophreniform disorder, schizoaffective disorder, delusional disorder, and shared psychotic disorder. In some embodiments, the schizophrenia is selected from catastrophic schizophrenia, catatonic schizophrenia, paranoid schizophrenia, residual schizophrenia, disorganized schizophrenia, and undifferentiated schizophrenia. In some embodiments, the disorder is selected from schizoid personality disorder, schizotypal personality disorder, and paranoid personality disorder. In some embodiments, the psychotic disorder is due to a general medical condition and is substance-induced or drug-induced (phencyclidine, ketamine and other dissociative anesthetics, amphetamine and other psychostimulants, and cocaine).

In some embodiments, the present disclosure provides a method for treating a cognitive disorder, comprising administering to a patient in need thereof an effective amount of a compound or a composition of the present disclosure. In some embodiments, cognitive disorders include dementia (associated with Alzheimer's disease, ischemia, multi-infarct dementia, trauma, vascular problems or stroke, HIV disease, Parkinson's disease, Huntington's disease, Pick's disease, Creutzfeldt-Jacob disease, perinatal hypoxia, other general medical conditions or substance abuse), delirium, amnestic disorder, substance-induced persisting delirium, dementia due to HIV disease, dementia due to Huntington's disease, dementia due to Parkinson's disease, Parkinsonian-ALS demential complex, dementia of the Alzheimer's type, age-related cognitive decline, and mild cognitive impairment.

The text revision of the fourth edition of the Diagnostic and Statistical Manual of Mental Disorders (DSM-IV-TR) (2000, American Psychiatric Association, Washington DC) provides a diagnostic tool that includes cognitive disorders including dementia, delirium, amnestic disorders and age-related cognitive decline. The fifth edition of the Diagnostic and Statistical Manual of Mental Disorders (DSM-5) (2013, American Psychiatric Association, Washington DC) provides a diagnostic tool for neurocognitive disorders (NCDs) that include delirium, followed by the syndromes of major NCD, mild NCD, and their etiological subtypes. The major or mild NCD subtypes include NCD due to Alzheimer's disease, vascular NCD, NCD with Lewy bodies, NCD due to Parkinson's disease, frontotemporal NCD, NCD due to traumatic brain injury, NCD due to HIV infection, substance/medication-induced NCD, NCD due to Huntington's disease, NCD due to prion disease, NCD due to another medical condition, NCD due to multiple etiologies, and unspecified NCD. The NCD category in DSM-5 encompasses the group of disorders in which the primary clinical deficit is in cognitive function, and that are acquired rather than developmental. As used herein, the term “cognitive disorders” includes treatment of those cognitive disorders and neurocognitive disorders as described in DSM-IV-TR or DSM-5. The skilled artisan will recognize that there are alternative nomenclatures, nosologies and classification systems for mental disorders, and that these systems evolve with medical and scientific progress. Thus the term “cognitive disorders” is intended to include like disorders that are described in other diagnostic sources.

In some embodiments, the present disclosure provides a method for treating schizophrenia or psychosis, comprising administering to a patient in need thereof an effective amount of a compound or composition of the present disclosure. Particular schizophrenia or psychosis pathologies are paranoid, disorganized, catatonic or undifferentiated schizophrenia and substance-induced psychotic disorder. DSM-IV-TR provides a diagnostic tool that includes paranoid, disorganized, catatonic, undifferentiated or residual schizophrenia, and substance-induced psychotic disorder. DSM-5 eliminated the subtypes of schizophrenia, and instead includes a dimensional approach to rating severity for the core symptoms of schizophrenia, to capture the heterogeneity in symptom type and severity expressed across individuals with psychotic disorders. As used herein, the term “schizophrenia or psychosis” includes treatment of those mental disorders as described in DSM-IV-TR or DSM-5. The skilled artisan will recognize that there are alternative nomenclatures, nosologies and classification systems for mental disorders, and that these systems evolve with medical and scientific progress. Thus the term “schizophrenia or psychosis” is intended to include like disorders that are described in other diagnostic sources.

In some embodiments, the present disclosure provides a method for treating pain, comprising administering to a patient in need thereof an effective amount of a compound or composition of the present disclosure. Particular pain embodiments are bone and joint pain (osteoarthritis), repetitive motion pain, dental pain, cancer pain, myofascial pain (muscular injury, fibromyalgia), perioperative pain (general surgery, gynecological), chronic pain and neuropathic pain.

The compounds and compositions may be further useful in a method for the prevention, treatment, control, amelioration, or reduction of risk of the diseases, disorders and conditions noted herein. The compounds and compositions may be further useful in a method for the prevention, treatment, control, amelioration, or reduction of risk of the aforementioned diseases, disorders and conditions, in combination with other agents.

In the treatment of conditions which require activation of mAChR M₄, an appropriate dosage level may be about 0.01 to 500 mg per kg patient body weight per day, which can be administered in single or multiple doses. The dosage level may be about 0.1 to about 250 mg/kg per day, or about 0.5 to about 100 mg/kg per day. A suitable dosage level can be about 0.01 to 250 mg/kg per day, about 0.05 to 100 mg/kg per day, or about 0.1 to 50 mg/kg per day. Within this range the dosage can be 0.05 to 0.5, 0.5 to 5 or 5 to 50 mg/kg per day. For oral administration, the compositions may be provided in the form of tablets containing 1.0 to 1000 milligrams of the active ingredient, particularly 1.0, 5.0, 10, 15, 20, 25, 50, 75, 100, 150, 200, 250, 300, 400, 500, 600, 750, 800, 900, or 1000 milligrams of the active ingredient for the symptomatic adjustment of the dosage to the patientto be treated. The compounds can be administered on a regimen of 1 to 4 times per day, preferably once or twice per day. This dosage regimen can be adjusted to provide the optimal therapeutic response. It will be understood, however, that the specific dose level and frequency of dosage for any particular patient can be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Thus, in some embodiments, the disclosure relates to a method for activating mAChR M₄ receptor activity in at least one cell, comprising the step of contacting the at least one cell with at least one disclosed compound or at least one product of a disclosed method in an amount effective to activate mAChR M₄ in the at least one cell. In some embodiments, the cell is mammalian, for example, human. In some embodiments, the cell has been isolated from a subject prior to the contacting step. In some embodiments, contacting is via administration to a subject.

In some embodiments, the invention relates to a method for activating mAChR M₄ activity in a subject, comprising the step of administering to the subject at least one disclosed compound or at least one product of a disclosed method in a dosage and amount effective to activating mAChR M₄ activity in the subject. In some embodiments, the subject is mammalian, for example, human. In some embodiments, the mammal has been diagnosed with a need for mAChR M₄ agonism prior to the administering step. In some embodiments, the mammal has been diagnosed with a need for mAChR M₄ activation prior to the administering step. In some embodiments, the method further comprises the step of identifying a subject in need of mAChR M₄ agonism.

In some embodiments, the invention relates to a method for the treatment of a disorder associated with selective mAChR M₄ activation, for example, a disorder associated with cholinergic activity, in a mammal comprising the step of administering to the mammal at least one disclosed compound or at least one product of a disclosed method in a dosage and amount effective to treat the disorder in the mammal. In some embodiments, the mammal is a human. In some embodiments, the mammal has been diagnosed with a need for treatment for the disorder prior to the administering step. In some embodiments, the method further comprises the step of identifying a subject in need of treatment for the disorder.

In some embodiments, the disorder can be selected from psychosis, schizophrenia, conduct disorder, disruptive behavior disorder, bipolar disorder, psychotic episodes of anxiety, anxiety associated with psychosis, psychotic mood disorders such as severe major depressive disorder; mood disorders associated with psychotic disorders, acute mania, depression associated with bipolar disorder, mood disorders associated with schizophrenia, behavioral manifestations of mental retardation, autistic disorder, movement disorders, Tourette's syndrome, akinetic-rigid syndrome, movement disorders associated with Parkinson's disease, tardive dyskinesia, drug induced and neurodegeneration based dyskinesias, attention deficit hyperactivity disorder, cognitive disorders, dementias, and memory disorders.

In some embodiments, the disorder is Alzheimer's disease.

b. Potentiation of Muscarinic Acetylcholine Receptor Activity

In some embodiments, the disclosure relates to a method for potentiation of muscarinic acetylcholine receptor activity in a mammal comprising the step of administering to the mammal an effective amount of at least one disclosed compound or a pharmaceutically acceptable salt thereof, or a pharmaceutical composition comprising at least one disclosed compound or pharmaceutically acceptable salt thereof.

In some embodiments, the disclosure provides a compound of formula (I), or a pharmaceutically acceptable salt thereof, for use in a method for the potentiation of muscarinic acetylcholine receptor activity in a mammal. In some embodiments, the disclosure provides a pharmaceutical composition comprising a compound of formula (I), or a pharmaceutically acceptable salt thereof, for use in a method for the potentiation of muscarinic acetylcholine receptor activity in a mammal.

In some embodiments, the disclosure provides a compound of formula (I), or a pharmaceutically acceptable salt thereof, for use in the manufacture of a medicament for the potentiation of muscarinic acetylcholine receptor activity in a mammal.

In some embodiments, potentiation of muscarinic acetylcholine receptor activity increases muscarinic acetylcholine receptor activity. In some embodiments, potentiation of muscarinic acetylcholine receptor activity is partial agonism of the muscarinic acetylcholine receptor. In some embodiments, potentiation of muscarinic acetylcholine receptor activity is positive allosteric modulation of the muscarinic acetylcholine receptor.

In some embodiments, the compound administered exhibits potentiation of mAChR M₄ with an EC₅₀ of less than about 10 μM, less than about 5 μM, less than about 1 μM, less than about 500 nM, or less than about 100 nM. In some embodiments, the compound administered exhibits potentiation of mAChR M₄ with an EC₅₀ of between about 10 μM and about 1 nM, about 1 μM and about 1 nM, about 100 nM and about 1 nM, or about 10 nM and about 1 nM.

In some embodiments, the mammal is a human. In some embodiments, the mammal has been diagnosed with a need for potentiation of muscarinic acetylcholine receptor activity prior to the administering step. In some embodiments, the method further comprises the step of identifying a mammal in need of potentiating muscarinic acetylcholine receptor activity. In some embodiments, the potentiation of muscarinic acetylcholine receptor activity treats a disorder associated with muscarinic acetylcholine receptor activity in the mammal. In some embodiments, the muscarinic acetylcholine receptor is mAChR M₄.

In some embodiments, potentiation of muscarinic acetylcholine receptor activity in a mammal is associated with the treatment of a neurological and/or psychiatric disorder associated with a muscarinic receptor dysfunction, such as a neurological or psychiatric disorder disclosed herein. In some embodiments, the muscarinic receptor is mAChR M₄.

In some embodiments, the disclosure provides to a method for potentiation of muscarinic acetylcholine receptor activity in a cell, comprising the step of contacting the cell with an effective amount of at least one disclosed compound or a pharmaceutically acceptable salt thereof. In some embodiments, the cell is mammalian (e.g., human). In some embodiments, the cell has been isolated from a mammal prior to the contacting step. In some embodiments, contacting is via administration to a mammal.

c. Enhancing Cognition

In some embodiments, the invention relates to a method for enhancing cognition in a mammal comprising the step of administering to the mammal an effective amount of least one disclosed compound; or a pharmaceutically acceptable salt, hydrate, solvate, or polymorph thereof.

In some embodiments, the disclosure provides a compound of formula (I), or a pharmaceutically acceptable salt thereof, for use in a method for the enhancement of cognition in a mammal. In some embodiments, the disclosure provides a pharmaceutical composition comprising a compound of formula (I), or a pharmaceutically acceptable salt thereof, for use in a method for the enhancement of cognition in a mammal.

In some embodiments, the disclosure provides a compound of formula (I), or a pharmaceutically acceptable salt thereof, for use in the manufacture of a medicament for the enhancment of cognition in a mammal.

In some embodiments, the mammal is a human. In some embodiments, the mammal has been diagnosed with a need for cognition enhancement prior to the administering step. In some embodiments, the method further comprises the step of identifying a mammal in need of cognition enhancement. In some embodiments, the need for cognition enhancement is associated with a muscarinic receptor dysfunction. In some embodiments, the muscarinic receptor is mAChR M₄.

In some embodiments, the cognition enhancement is a statistically significant increase in Novel Object Recognition. In some embodiments, the cognition enhancement is a statistically significant increase in performance of the Wisconsin Card Sorting Test.

d. Cotherapeutic Methods

The present invention is further directed to administration of a selective mAChR M₄ activator for improving treatment outcomes in the context of cognitive or behavioral therapy. That is, in some embodiments, the invention relates to a cotherapeutic method comprising a step of administering to a mammal an effective amount and dosage of at least one disclosed compound, or a pharmaceutically acceptable salt thereof.

In some embodiments, the disclosure provides a compound of formula (I), or a pharmaceutically acceptable salt thereof, for use in a cotherapeutic method with cognitive or behaviorial therapy in a mammal. In some embodiments, the disclosure provides a pharmaceutical composition comprising a compound of formula (I), or a pharmaceutically acceptable salt thereof, for use in a cotherapeutic method with cognitive or behaviorial therapy in a mammal.

In some embodiments, the disclosure provides a compound of formula (I), or a pharmaceutically acceptable salt thereof, for use in the manufacture of a medicament for a cotherapeutic method with cognitive or behaviorial therapy in a mammal.

In some embodiments, administration improves treatment outcomes in the context of cognitive or behavioral therapy. Administration in connection with cognitive or behavioral therapy can be continuous or intermittent. Administration need not be simultaneous with therapy and can be before, during, and/or after therapy. For example, cognitive or behavioral therapy can be provided within 1, 2, 3, 4, 5, 6, 7 days before or after administration of the compound. As a further example, cognitive or behavioral therapy can be provided within 1, 2, 3, or 4 weeks before or after administration of the compound. As a still further example, cognitive or behavioral therapy can be provided before or after administration within a period of time of 1, 2, 3, 4, 5, 6, 7, 8, 9, or 10 half-lives of the administered compound.

It is understood that the disclosed cotherapeutic methods can be used in connection with the disclosed compounds, compositions, kits, and uses.

e. Combination Therapies

In the methods of use described herein, additional therapeutic agent(s) may be administered simultaneously or sequentially with the disclosed compounds and compositions. Sequential administration includes administration before or after the disclosed compounds and compositions. In some embodiments, the additional therapeutic agent or agents may be administered in the same composition as the disclosed compounds. In other embodiments, there may be an interval of time between administration of the additional therapeutic agent and the disclosed compounds. In some embodiments, administration of an additional therapeutic agent with a disclosed compound may allow lower doses of the other therapeutic agents and/or administration at less frequent intervals. When used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula (I). The above combinations include combinations of a compound of the present invention not only with one other active compound, but also with two or more other active compounds.

The disclosed compounds can be used as single agents or in combination with one or more other drugs in the treatment, prevention, control, amelioration or reduction of risk of the aforementioned diseases, disorders and conditions for which the compound or the other drugs have utility, where the combination of drugs together are safer or more effective than either drug alone. The other drug(s) can be administered by a route and in an amount commonly used therefor, contemporaneously or sequentially with a disclosed compound. When a disclosed compound is used contemporaneously with one or more other drugs, a pharmaceutical composition in unit dosage form containing such drugs and the disclosed compound may be used. However, the combination therapy can also be administered on overlapping schedules. It is also envisioned that the combination of one or more active ingredients and a disclosed compound can be more efficacious than either as a single agent. Thus, when used in combination with one or more other active ingredients, the disclosed compounds and the other active ingredients can be used in lower doses than when each is used singly.

The pharmaceutical compositions and methods of the present invention can further comprise other therapeutically active compounds as noted herein which are usually applied in the treatment of the above mentioned pathological conditions.

The above combinations include combinations of a disclosed compound not only with one other active compound, but also with two or more other active compounds. Likewise, disclosed compounds can be used in combination with other drugs that are used in the prevention, treatment, control, amelioration, or reduction of risk of the diseases or conditions for which disclosed compounds are useful. Such other drugs can be administered, by a route and in an amount commonly used therefor, contemporaneously or sequentially with a compound of the present invention. When a compound of the present invention is used contemporaneously with one or more other drugs, a pharmaceutical composition containing such other drugs in addition to a disclosed compound is preferred. Accordingly, the pharmaceutical compositions include those that also contain one or more other active ingredients, in addition to a compound of the present invention.

The weight ratio of a disclosed compound to the second active ingredient can be varied and will depend upon the effective dose of each ingredient. Generally, an effective dose of each will be used. Thus, for example, when a compound of the present invention is combined with another agent, the weight ratio of a disclosed compound to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations a disclosed compound and other active agents can be administered separately or in conjunction. In addition, the administration of one element can be prior to, concurrent to, or subsequent to the administration of other agent(s).

Accordingly, the disclosed compounds can be used alone or in combination with other agents which are known to be beneficial in the subject indications or other drugs that affect receptors or enzymes that either increase the efficacy, safety, convenience, or reduce unwanted side effects or toxicity of the disclosed compounds. The subject compound and the other agent can be coadministered, either in concomitant therapy or in a fixed combination.

In some embodiments, the compound can be employed in combination with anti-Alzheimer's agents, beta-secretase inhibitors, cholinergic agents, gamma-secretase inhibitors, HMG-CoA reductase inhibitors, M₁ allosteric agonists, M₁ positive allosteric modulators, NSAIDs including ibuprofen, vitamin E, and anti-amyloid antibodies. In another embodiment, the subject compound can be employed in combination with sedatives, hypnotics, anxiolytics, antipsychotics (typical and atypical), antianxiety agents, cyclopyrrolones, imidazopyridines, pyrazolopyrimidines, minor tranquilizers, melatonin agonists and antagonists, melatonergic agents, benzodiazepines, barbiturates, 5HT-2 antagonists, and the like, such as: adinazolam, allobarbital, alonimid, alprazolam, amisulpride, amitriptyline, amobarbital, amoxapine, aripiprazole, bentazepam, benzoctamine, brotizolam, bupropion, busprione, butabarbital, butalbital, capuride, carbocloral, chloral betaine, chloral hydrate, clomipramine, clonazepam, cloperidone, clorazepate, chlordiazepoxide, clorethate, chlorpromazine, clozapine, cyprazepam, desipramine, dexclamol, diazepam, dichloralphenazone, divalproex, diphenhydramine, doxepin, estazolam, ethchlorvynol, etomidate, fenobam, flunitrazepam, flupentixol, fluphenazine, flurazepam, fluvoxamine, fluoxetine, fosazepam, glutethimide, halazepam, haloperidol, hydroxyzine, imipramine, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, olanzapine, oxazepam, paraldehyde, paroxetine, pentobarbital, perlapine, perphenazine, phenelzine, phenobarbital, prazepam, promethazine, propofol, protriptyline, quazepam, quetiapine, reclazepam, risperidone, roletamide, secobarbital, sertraline, suproclone, temazepam, thioridazine, thiothixene, tracazolate, tranylcypromaine, trazodone, triazolam, trepipam, tricetamide, triclofos, trifluoperazine, trimetozine, trimipramine, uldazepam, venlafaxine, zaleplon, ziprasidone, zolazepam, zolpidem, and salts thereof, and combinations thereof, and the like, or the subject compound can be administered in conjunction with the use of physical methods such as with light therapy or electrical stimulation.

In some embodiments, the compound can be employed in combination with levodopa (with or without a selective extracerebral decarboxylase inhibitor such as carbidopa or benserazide), anticholinergics such as biperiden (optionally as its hydrochloride or lactate salt) and trihexyphenidyl (benzhexol) hydrochloride, COMT inhibitors such as entacapone, MOA-B inhibitors, antioxidants, A2a adenosine receptor antagonists, cholinergic agonists, NMDA receptor antagonists, serotonin receptor antagonists and dopamine receptor agonists such as alentemol, bromocriptine, fenoldopam, lisuride, naxagolide, pergolide and pramipexole. It will be appreciated that the dopamine agonist can be in the form of a pharmaceutically acceptable salt, for example, alentemol hydrobromide, bromocriptine mesylate, fenoldopam mesylate, naxagolide hydrochloride and pergolide mesylate. Lisuride and pramipexol are commonly used in a non-salt form.

In some embodiments, the compound can be employed in combination with a compound from the phenothiazine, thioxanthene, heterocyclic dibenzazepine, butyrophenone, diphenylbutylpiperidine and indolone classes of neuroleptic agent. Suitable examples of phenothiazines include chlorpromazine, mesoridazine, thioridazine, acetophenazine, fluphenazine, perphenazine and trifluoperazine. Suitable examples of thioxanthenes include chlorprothixene and thiothixene. An example of a dibenzazepine is clozapine. An example of a butyrophenone is haloperidol. An example of a diphenylbutylpiperidine is pimozide. An example of an indolone is molindolone. Other neuroleptic agents include loxapine, sulpiride and risperidone. It will be appreciated that the neuroleptic agents when used in combination with the subject compound can be in the form of a pharmaceutically acceptable salt, for example, chlorpromazine hydrochloride, mesoridazinebesylate, thioridazine hydrochloride, acetophenazine maleate, fluphenazine hydrochloride, flurphenazine enathate, fluphenazine decanoate, trifluoperazine hydrochloride, thiothixene hydrochloride, haloperidol decanoate, loxapine succinate and molindone hydrochloride. Perphenazine, chlorprothixene, clozapine, haloperidol, pimozide and risperidone are commonly used in a non-salt form. Thus, the subject compound can be employed in combination with acetophenazine, alentemol, aripiprazole, amisulpride, benzhexol, bromocriptine, biperiden, chlorpromazine, chlorprothixene, clozapine, diazepam, fenoldopam, fluphenazine, haloperidol, levodopa, levodopa with benserazide, levodopa with carbidopa, lisuride, loxapine, mesoridazine, molindolone, naxagolide, olanzapine, pergolide, perphenazine, pimozide, pramipexole, quetiapine, risperidone, sulpiride, tetrabenazine, trihexyphenidyl, thioridazine, thiothixene, trifluoperazine or ziprasidone.

In some embodiments, the compound can be employed in combination with an anti-depressant or anti-anxiety agent, including norepinephrine reuptake inhibitors (including tertiary amine tricyclics and secondary amine tricyclics), selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, neurokinin-1 receptor antagonists, atypical anti-depressants, benzodiazepines, 5-HT1A agonists or antagonists, especially 5-HT1A partial agonists, and corticotropin releasing factor (CRF) antagonists. Specific agents include: amitriptyline, clomipramine, doxepin, imipramine and trimipramine; amoxapine, desipramine, maprotiline, nortriptyline and protriptyline; fluoxetine, fluvoxamine, paroxetine and sertraline; isocarboxazid, phenelzine, tranylcypromine and selegiline; moclobemide: venlafaxine; duloxetine; aprepitant; bupropion, lithium, nefazodone, trazodone and viloxazine; alprazolam, chlordiazepoxide, clonazepam, chlorazepate, diazepam, halazepam, lorazepam, oxazepam and prazepam; buspirone, flesinoxan, gepirone and ipsapirone, and pharmaceutically acceptable salts thereof.

In some embodiments, the compounds can be coadministered with orthosteric muscarinic agonists, muscarinic potentiators, or cholinesterase inhibitors. In some embodiments, the compounds can be coadministered with GlyT1 inhibitors and the like such as, but not limited to: risperidone, clozapine, haloperidol, fluoxetine, prazepam, xanomeline, lithium, phenobarbitol, and salts thereof and combinations thereof.

f. Modes of Administration

Methods of treatment may include any number of modes of administering a disclosed composition. Modes of administration may include tablets, pills, dragees, hard and soft gel capsules, granules, pellets, aqueous, lipid, oily or other solutions, emulsions such as oil-in-water emulsions, liposomes, aqueous or oily suspensions, syrups, elixirs, solid emulsions, solid dispersions or dispersible powders. For the preparation of pharmaceutical compositions for oral administration, the agent may be admixed with commonly known and used adjuvants and excipients such as for example, gum arabic, talcum, starch, sugars (such as, e.g., mannitose, methyl cellulose, lactose), gelatin, surface-active agents, magnesium stearate, aqueous or non-aqueous solvents, paraffin derivatives, cross-linking agents, dispersants, emulsifiers, lubricants, conserving agents, flavoring agents (e.g., ethereal oils), solubility enhancers (e.g., benzyl benzoate or benzyl alcohol) or bioavailability enhancers (e.g. Gelucire™). In the pharmaceutical composition, the agent may also be dispersed in a microparticle, e.g. a nanoparticulate composition.

For parenteral administration, the agent can be dissolved or suspended in a physiologically acceptable diluent, such as, e.g., water, buffer, oils with or without solubilizers, surface-active agents, dispersants or emulsifiers. As oils for example and without limitation, olive oil, peanut oil, cottonseed oil, soybean oil, castor oil and sesame oil may be used. More generally spoken, for parenteral administration, the agent can be in the form of an aqueous, lipid, oily or other kind of solution or suspension or even administered in the form of liposomes or nano-suspensions.

The term “parenterally,” as used herein, refers to modes of administration which include intravenous, intramuscular, intraperitoneal, intrasternal, subcutaneous and intraarticular injection and infusion.

### 5. KITS

In one aspect, the disclosure provides kits comprising at least one disclosed compound or a pharmaceutically acceptable salt thereof, and one or more of:
(a) at least one agent known to increase mAChR M₄ activity;
(b) at least one agent known to decrease mAChR M₄ activity;
(c) at least one agent known to treat a disorder associated with cholinergic activity;
(d) instructions for treating a disorder associated with cholinergic activity;
(e) instructions for treating a disorder associated with M₄ receptor activity; or
(f) instructions for administering the compound in connection with cognitive or behavioral therapy.

In some embodiments, the at least one disclosed compound and the at least one agent are co-formulated. In some embodiments, the at least one disclosed compound and the at least one agent are co-packaged. The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

The disclosed kits can be employed in connection with disclosed methods of use.

The kits may further comprise information, instructions, or both that use of the kit may provide treatment for medical conditions in mammals (particularly humans). The information and instructions may be in the form of words, pictures, or both, and the like. In addition or in the alternative, the kit may include the compound, a composition, or both; and information, instructions, or both; regarding methods of application of compound, or of composition, for example with the benefit of treating or preventing medical conditions in mammals (e.g., humans).

The compounds and processes of the invention will be better understood by reference to the following examples, which are intended as an illustration of and not a limitation upon the scope of the invention.

### 6. EXAMPLES

All NMR spectra were recorded on a 400 MHz AMX Bruker NMR spectrometer. ¹H chemical shifts are reported in 6 values in ppm downfield with the deuterated solvent as the internal standard. Data are reported as follows: chemical shift, multiplicity (s=singlet, bs=broad singlet, d=doublet, t=triplet, q=quartet, dd=doublet of doublets, m=multiplet, ABq=AB quartet), coupling constant, integration. Reversed-phase LCMS analysis was performed using an Agilent 1200 system comprised of a binary pump with degasser, high-performance autosampler, thermostatted column compartment, C18 column, diode-array detector (DAD) and an Agilent 6150 MSD with the following parameters. The gradient conditions were 5% to 95% acetonitrile with the aqueous phase 0.1% TFA in water over 1.4 minutes, hold at 95% acetonitrile for 0.1 min, 0.5 mL/min, 55° C. (“90 sec method”). Samples were separated on a Waters Acquity UPLC BEH C18 column (1.7 μm, 1.0×50 mm) at 0.5 mL/min, with column and solvent temperatures maintained at 55° C. The DAD was set to scan from 190 to 300 nm, and the signals used were 220 nm and 254 nm (both with a band width of 4 nm). The MS detector was configured with an electrospray ionization source, and the low-resolution mass spectra were acquired by scanning from 140 to 700 AMU with a step size of 0.2 AMU at 0.13 cycles/second, and peak width of 0.008 minutes. The drying gas flow was set to 13 liters per minute at 300° C. and the nebulizer pressure was set to 30 psi. The capillary needle voltage was set at 3000 V, and the fragmentor voltage was set at 100V. Data acquisition was performed with Agilent Chemstation and Analytical Studio Reviewer software.

Abbreviations used in the examples and reaction schemes that follow include the following:
aq is aqueous;
BINAP is 2,2′-bis(diphenylphosphino)-1,1′-binaphthyl;
Boc is tert-butoxycarbonyl;
BrettPhos is 2-(dicy clohexylphosphino)3,6-dimethoxy-2′,4′,6′-triisopropyl-1,1′-biphenyl;
BrettPhos Pd G3 is [(2-di-cy clohexylphosphino-3,6-dimethoxy-2′,4′,6′-triisopropyl-1,1′-biphenyl)-2-(2′-amino-1,1′-biphenyl)]palladium(II) methanesulfonate;
t-BuOH is tert-butanol;
DCE is 1,2-dichloroethane;
DCM is dichloromethane;
DEA is diethylamine;
DMF is N,N-dimethylformamide;
DMP or Dess-Martin periodinane is 1,1,1-tris(acetyloxy)-1,1-dihydro-1,2-benziodoxol-3-(1H)-one;
DIAD is diisopropyl azodicarboxylate;
DIPEA or DIEA is diisopropylethylamine;
DMAP is 4-dimethylaminopyridine;
DMSO is dimethyl sulfoxide;
Dowtherm™ A is a eutectic mixture of 26.5% diphenyl+73.5% diphenyl oxide;
DtBAD is di-tert-butyl-azodicarboxylate;
eq or eq. is equivalent(s);
EtOAC is ethyl acetate;
(4,4′-dtbbpy)NiCl₂ is 4,4′-bis(1,1-dimethylethyl)-2,2′-bipyridine]nickel (II) dichloride;
EtOH is ethanol;
h or hr is hour(s);
Hex is hexane(s);
IPA is isopropyl alcohol;
KOAc is potassium acetate;
LAH is lithium aluminum hydride;
mCPBA is meta-chloroperoxy benzoic acid;
MeCN or ACN is acetonitrile;
MeOH is methanol;
min is minute(s);
NaOAc is sodium acetate;
NaOMe is sodium methoxide;
NBS is N-bromosuccinimide;
NCS is N-chlorosuccinimide;
NMP is N-methyl-2-pyrrolidone;
[Pd(allyl)(tBuBrettPhos)]OTf is trifluoromethanesulfonate allyl[(2-Di-tert-butylphosphino-3,6-dimethoxy-2′,4′,6′-triisopropyl-1,1′-biphenyl)-2-(2′-amino-1,1′-biphenyl)]palladium(II)
Pd(dppf)Cl₂ is [1,1′-bis(diphenylphosphino)ferrocene]dichloropalladium(II);
Pd(OAc)₂ is palladium(II)acetate;
PPA is polyphosphoric acid;
PPh₃ is triphenylphosphine;
PPTS is pyridinium p-toluenesulfonate;
rt is room temperature;
sat. is saturated;
sec is second(s);
SCX cartridge or HF SCX cartridge is a strong cation exchanger cartridge (i.e. Agilentpart #14256027);
SFC is supercritical fluid chromatography;
TBAC or TBACl is tetrabutylammonium chloride;
t-BuXPhos is 2-di-tert-butylphosphino-2′,4′,6′-triisopropylbiphenyl;
TEA or Et₃N is triethyamine;
TFA is trifluoroacetic acid;
THF is tetrahydrofuran;
TosCl is para-toluenesulfonyl chloride; and
tosyl is para-toluenesulfonyl.
   a. Preparation of Intermediates

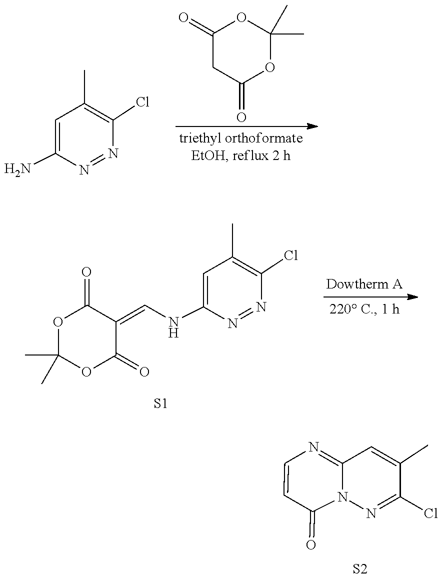

5-(((6-Chloro-5-methylpyridazin-3-yl)amino)methylene)-2,2-dimethyl-1,3-dioxane-4,6-dione (Intermediate S1): To a solution of 6-chloro-5-methylpyridazin-3-amine (1 g) in ethanol (12 mL) were added triethyl orthoformate (1.16 mL) and isopropylidene malonate (1.04 g) at ambient temperature. The mixture was then heated to 60° C. for 18 hours. The mixture was filtered and the cake was washed with ethanol (5 mL×3) to afford the intermediate 5-[[(6-chloro-5-methyl-pyridazin-3-yl)amino]methylene]-2,2-dimethyl-1,3-dioxane-4,6-dione (1.59 g). ES-MS [M+1]⁺: 298.2

7-Chloro-8-methyl-4H-pyrimido[1,2-b]pyridazin-4-one (Intermediate S2): A solution of 5-[[(6-chloro-5-methyl-pyridazin-3-yl)amino]methylene]-2,2-dimethyl-1,3-dioxane-4,6-dione (1.59 g) in Dowtherm™ A (8 mL) was stirred at 220° C. for 1 hour. After cooling, the mixture was added to water and acidified using 1M HCl. The aqueous layer was extracted with hexanes (3×125 mL). The aqueous layer was neutralized with aq. NaHCO₃ and the mixture was extracted with chloroform/IPA (4:1) (3×). The combined organic layers were dried over magnesium sulfate and concentrated to give the title compound (908 mg). ¹H NMR (400 MHz, DMSO) δ 8.23 (d, J=6.5 Hz, 1H), 8.03 (q, J=1.3 Hz, 1H), 6.54 (d, J=6.4 Hz, 1H), 2.44 (d, J=1.3 Hz, 3H). ES-MS [M+1]⁺: 196.

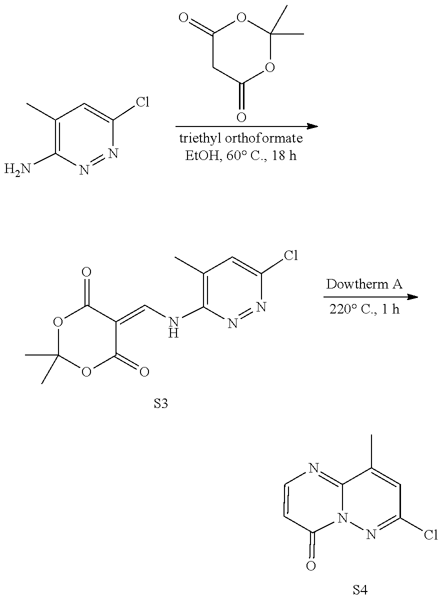

5-(((6-Chloro-4-methylpyridazin-3-yl)amino)methylene)-2,2-dimethyl-1,3-dioxane-4,6-dione (Intermediate S3): To a solution of 6-chloro-4-methylpyridazin-3-amine (100 mg) in ethanol (1.2 mL) were added triethyl orthoformate (174 L) and isopropylidene malonate (151 mg) at ambient temperature. The mixture was then heated to 60° C. for 18 hours. The mixture was filtered and the cake was washed with ethanol (5 mL×3) to afford the title compound which was carried forward without further purification. ES-MS [M+1]⁺: 298.1.

7-Chloro-9-methyl-4H-pyrimido[1,2-b]pyridazin-4-one (Intermediate S4): A solution of 5-(((6-chloro-4-methylpyridazin-3-yl)amino)methylene)-2,2-dimethyl-1,3-dioxane-4,6-dione in Dowtherm™ A (1 mL) was stirred at 220° C. for 1 hour. After cooling, the mixture was added to water and acidified using 1M HCl. The aqueous layer was extracted with hexanes (3×125 mL) to remove Dowtherm™ A. The aqueous layer was neutralized with aqueous NaHCO₃ and the mixture was extracted with chloroform/IPA (4:1) (3×). The combined organic layers were dried over magnesium sulfate, filtered, and concentrated to give 22 mg of the title compound. ¹H NMR (400 MHz, CD₃OD) δ 8.34 (d, J=6.4 Hz, 1H), 7.68 (q, J=1.3 Hz, 1H), 6.69 (d, J=6.4 Hz, 1H), 2.60 (d, J=1.3 Hz, 3H); ES-MS [M+1]⁺: 196.2.

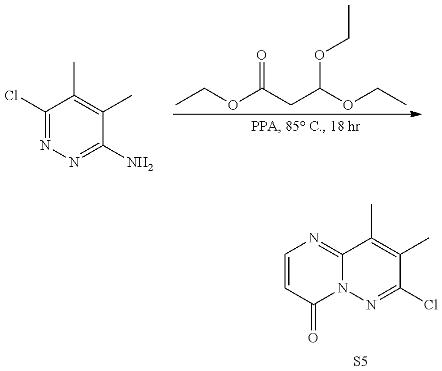

7-Chloro-8,9-dimethyl-pyrimido[1,2-b]pyridazin-4-one (Intermediate S5): A mixture of 6-chloro-3-amino-4,5-dimethylpyridazine (550 mg), ethyl 3,3-diethoxypropionate (1.02 mL), and polyphosphoric acid (5 mL) was added to a sealed tube. The mixture was stirred at 85° C. for 5 hours, then cooled to room temperature and additional ethyl 3,3-diethoxypropionate (1.02 mL) was added to the reaction mixture. The vial was sealed and the mixture heated to 85° C. for 18 hours. The reaction mixture was then slowly added to a stirred solution of 150 mL of saturated aqueous NaHCO₃ and 100 mL of DCM. After complete addition, the mixture was allowed to stir for 20 minutes, the organic layer was separated, and the aqueous layer was further extracted with chloroform/IPA (4:1) (3×). The organic layers were pooled, dried over MgSO₄, filtered, and concentrated. The crude product was purified by normal phase chromatography (0-70% EtOAc/hexanes) to give the title compound (366 mg). ¹H NMR (400 MHz, CDCl₃) δ 8.22-8.20 (d, J=6.5 Hz, 1H), 6.64-6.62 (d, J=6.5 Hz, 1H), 2.62 (d, J=0.6 Hz, 3H), 2.51 (d, J=0.6 Hz, 3H). ES-MS [M+1]⁺: 210.

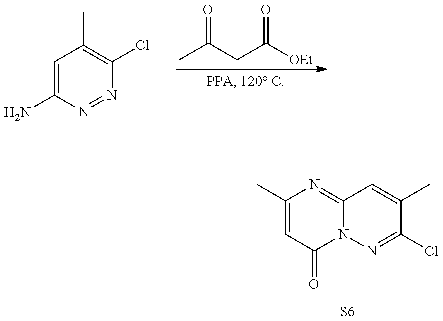

7-Chloro-2,8-dimethyl-4H-pyrimido[1,2-b]pyridazin-4-one (Intermediate S6): To a solution of 6-chloro-3-amino-5-methylpyridazine (1 g) in polyphosphoric acid (5 mL) was added ethyl acetoacetate (1.76 mL). The mixture was heated to 120° C. for 18 hours. While still hot, the mixture was slowly added to a stirred solution of 150 mL saturated NaHCO₃. The aqueous layer was extracted with chloroform:IPA (4:1) thrice. The organic layers were dried over magnesium sulfate, filtered, and concentrated under vacuum. The crude material was purified via flash column chromatography on silica gel (0-70% EtOAc/DCM) to afford 1.34 g of title compound. ¹H NMR (400 MHz, CDCl₃) δ 7.59 (q, J=1.3 Hz, 1H), 6.50 (s, 1H), 2.50 (d, J=1.3 Hz, 3H), 2.43 (d, J=0.7 Hz, 3H). ES-MS [M+1]⁺: 210.

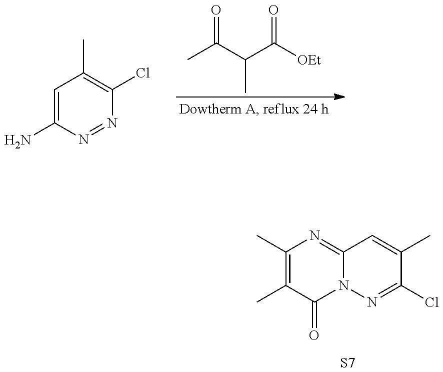

7-Chloro-2,3,8-trimethyl-4H-pyrimido[1,2-b]pyridazin-4-one (Intermediate S7). To a solution of 6-chloro-3-amino-5-methylpyridazine (500 mg) in Dowtherm™ A (2 mL) was added ethyl 2-methyl-3-oxo-butanoate (591 L) and the reaction was heated to 150° C. for 18 h. Additional ethyl 2-methyl-3-oxo-butanoate (591 L) was added and the reaction was irradiated in a microwave reactor at 180° C. for 15 minutes. The solution was directly purified via normal phase column chromatography on silica gel (100% Hex then 0-80% EtOAc/DCM) to afford 442 mg of title compound. ¹H NMR (400 MHz, CDCl₃) δ 7.53 (q, J=1.3 Hz, 1H), 2.47 (d, J=1.3 Hz, 3H), 2.45 (s, 3H), 2.26 (s, 3H). ES-MS [M+1]⁺: 224.

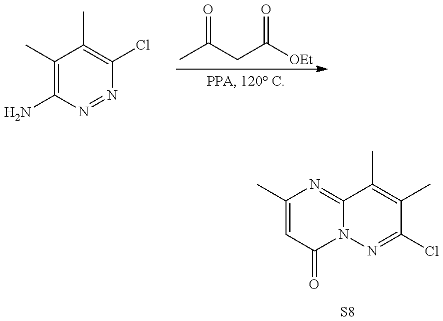

7-Chloro-2,8,9-trimethyl-4H-pyrimido[1,2-b]pyridazin-4-one (Intermediate S8): To a solution of 6-chloro-3-amino-4,5-dimethylpyridazine (500 mg) in polyphosphoric acid (4.5 mL) was added ethyl acetoacetate (803 L). The mixture was heated to 120° C. for 1 hour. While hot, the mixture was slowly added to 125 mL of saturated NaHCO₃ and 50 mL chloroform/IPA (4:1). Upon complete addition, the mixture was allowed to stir for 15 minutes and the organic layer was isolated. The aqueous layer was further extracted with chloroform:IPA (4:1) (×3). The organic layers were dried over magnesium sulfate, filtered, and concentrated under vacuum. The crude product was taken up in 5 mL MeOH, sonicated, and the precipitate was collected by vacuum filtration and washed with hexanes. The solid was dried under nitrogen to yield the title compound as a solid (472 mg). ¹H NMR (400 MHz, CDCl₃) δ 6.49 (s, 1H), 2.60 (d, J=0.8 Hz, 3H), 2.48 (q, J=0.7 Hz, 3H), 2.44 (q, J=0.5 Hz, 3H). ES-MS [M+1]⁺: 224.

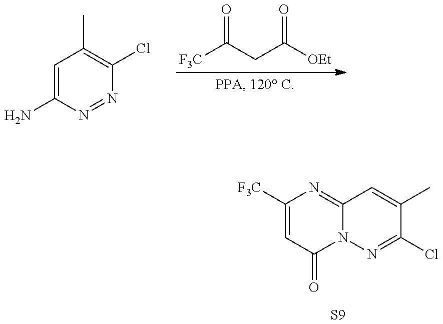

7-Chloro-8-methyl-2-(trifluoromethyl)-4H-pyrimido[1,2-b]pyridazin-4-one (Intermediate S9): To a solution of 6-chloro-3-amino-5-methylpyridazine (500 mg) in polyphosphoric acid (5 mL) was added ethyl 4,4,4-trifluoroacetoacetate (1.02 mL). The mixture was heated to 120° C. for 18 hours. While hot, the mixture was slowly added to a beaker containing 125 mL of saturated NaHCO₃ and 50 mL chloroform/IPA (4:1) while maintaining a pH-7. Upon complete addition, the mixture was allowed to stir for 15 minutes and the organic layer was isolated. The aqueous layer was further extracted with chloroform:IPA (4:1) thrice. The organic layers were dried over magnesium sulfate, filtered, and concentrated under vacuum. The crude product was dissolved in 15 mL of DMSO (any solids that precipitated were collected to give pure desired product) and the filtrate was purified by RP-HPLC (5-45% MeCN/0.05% aqueousNH₄OH). The fractions containing product were concentrated and combined with the previous solid to afford 393 mg of the title compound. ¹H NMR (400 MHz, CDCl₃) δ 7.79 (q, J=1.3 Hz, 1H), 6.98 (s, 1H), 2.56 (d, J=1.3 Hz, 3H). ES-MS [M+1]⁺: 264.

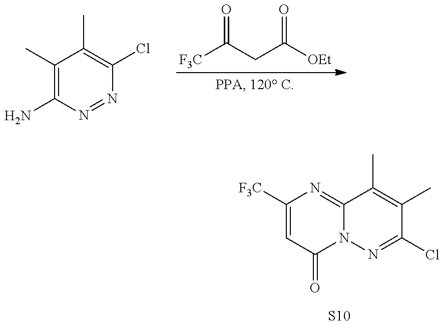

7-Chloro-8,9-dimethyl-2-(trifluoromethyl)-4H-pyrimido[1,2-b]pyridazin-4-one (Intermediate S10): To a solution of 6-chloro-3-amino-4,5-dimethylpyridazine (550 mg) in polyphosphoric acid (5 mL) was added ethyl 4,4,4-trifluoroacetoacetate (1.02 mL). The mixture was heated to 120° C. for 18 hours. Additional ethyl 4,4,4-trifluoroacetoacetate (1.02 mL) was added and the mixture was stirred for an additional 7 hours at 120° C. While hot, the mixture was slowly added to a beaker containing 125 mL of saturated NaHCO₃ and 50 mL chloroform/IPA (4:1) while maintaining a pH ˜7. Upon complete addition, the mixture was allowed to stir for 15 minutes and the organic layer was isolated. The aqueous layer was further extracted with chloroform:IPA (4:1) thrice. The organic layers were dried over magnesium sulfate, filtered, and concentrated under vacuum. The crude product was dissolved in DMSO (16 mL) and purified by reverse-phase HPLC (5-50% ACN/0.05% aqueous NH₄OH). The fractions containing product were concentrated to afford the title compound (264 mg). ¹H NMR (400 MHz, CDCl₃) δ 6.96 (s, 1H), 2.57 (q, J=0.9 Hz, 3H), 2.48 (q, J=0.9 Hz, 3H). ES-MS [M+1]⁺: 278.

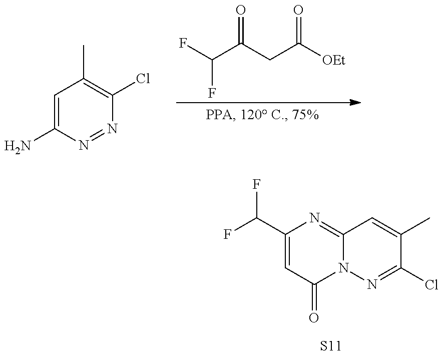

7-Chloro-2-(difluoromethyl)-8-methyl-4H-pyrimido[1,2-b]pyridazin-4-one (Intermediate S11): To a solution of 6-chloro-3-amino-5-methylpyridazine (500 mg) in polyphosphoric acid (5 mL) was added ethyl 4,4-difluoroacetoacetate (912 μL). The mixture was heated to 120° C. for 2 hours. While hot, the mixture was slowly added to a beaker containing 125 mL of saturated NaHCO₃ and 50 mL chloroform/IPA (4:1) while maintaining a pH˜7. Upon complete addition, the mixture was allowed to stir for 15 minutes and the organic layer was isolated. The aqueous layer was further extracted with chloroform:IPA (4:1) thrice. The organic layers were dried over magnesium sulfate, filtered, and concentrated under vacuum. The crude product was dissolved in DMSO (15 mL) and purified by RP-HPLC (0-45% MeCN/0.05% aqueous NH₄OH). The fractions containing product were concentrated to afford 640 mg of the title compound. ¹H NMR (400 MHz, CDCl₃) δ 7.73 (q, J=1.3 Hz, 1H), 6.90 (s, 1H), 6.46 (t, J=54.8 Hz, 1H), 2.55 (d, J=1.3 Hz, 3H). ES-MS [M+1]⁺: 246.

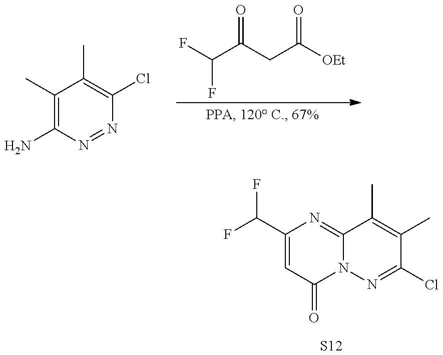

7-Chloro-2-(difluoromethyl)-8,9-dimethyl-4H-pyrimido[1,2-b]pyridazin-4-one (Intermediate S12): To a solution of 6-chloro-3-amino-4,5-dimethylpyridazine (1.65 g) in polyphosphoric acid (10 mL) was added ethyl 4,4-difluoroacetoacetate (2.74 mL). The mixture was heated to 120° C. for 6 hours. While hot, the reaction mixture was then slowly added into a stirred saturated NaHCO₃ solution (200 mL). Once the pH-7, the aqueous layer was extracted with chloroform:IPA (4:1). The organic layers were dried over magnesium sulfate, filtered, and concentrated under vacuum. The crude product was purified by normal phase chromatography (0-40% EtOAc/DCM) to afford the title compound (1.05 g). ¹H NMR (400 MHz, CDCl₃) δ 6.90 (s, 1H), 6.48 (t, J=54.9 Hz, 1H), 2.64 (s, 3H), 2.53 (s, 3H). ES-MS [M+1]⁺: 260.

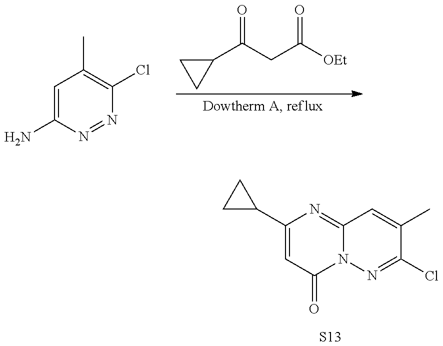

7-Chloro-2-cyclopropyl-8-methyl-4H-pyrimido[1,2-b]pyridazin-4-one (Intermediate S13). In a sealed vial was added a solution of 6-chloro-3-amino-5-methylpyridazine (500 mg) and ethyl 3-cyclopropyl-3-oxopropanoate (617 μL) in Dowtherm™ A (5 mL). The vial was sealed and the reaction was heated to 150° C. for 18 h. Additional ethyl 3-cyclopropyl-3-oxopropanoate (617 μL) was added and the reaction was heated to 220° C. for 3 h. The mixture was purified via normal phase column chromatography (100% hexanes; then 0-40% EtOAc/DCM) to afford 44 mg of title compound. ¹H NMR (400 MHz, CDCl₃) δ 7.52 (q, J=1.3 Hz, 1H), 6.51 (s, 1H), 2.47 (d, J=1.4 Hz, 3H), 1.97-1.86 (m, 1H), 1.18-1.10 (m, 2H), 1.08-0.97 (m, 2H). ES-MS [M+1]⁺: 236.

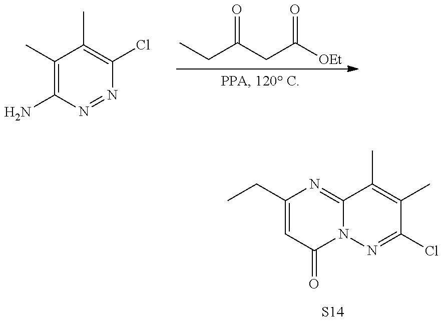

7-Chloro-2-ethyl-8,9-dimethyl-4H-pyrimido[1,2-b]pyridazin-4-one (Intermediate S14): To a solution of 6-chloro-3-amino-4,5-dimethylpyridazine (550 mg) in polyphosphoric acid (4 mL) was added ethyl 3-oxopentanoate (913 μL). The mixture was heated to 85° C. for 1 hour, then 120° C. for 1 hour. While hot, the mixture was slowly transferred to a stirred solution of saturated aqueous NaHCO₃ (˜150 mL) and chloroform/IPA (4:1) (50 mL). Upon complete addition, the mixture was stirred for 10 minutes then the organic layer was isolated. The aqueous layers were further extracted with chloroform/IPA (4:1) (3×) and the organic layers were pooled, dried over MgSO₄, filtered, and concentrated under vacuum. Upon cooling, a solid precipitate was observed. Hexanes were added, the suspension was sonicated, the solid was collected by vacuum filtration, and washed with hexanes to provide the title compound (469 mg) >95% clean by LCMS. ¹H NMR (400 MHz, CDCl₃) δ 6.50 (s, 1H), 2.70 (q, J=7.6 Hz, 2H), 2.61 (s, 3H), 2.48 (s, 3H), 1.30 (t, J=7.6 Hz, 3H). ES-MS [M+1]⁺: 238.

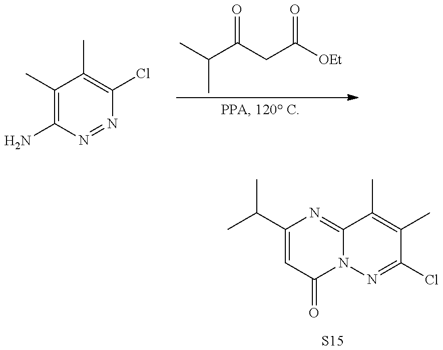

7-Chloro-2-isopropyl-8,9-dimethyl-4H-pyrimido[1,2-b]pyridazin-4-one (Intermediate S15): To a solution of 6-chloro-3-amino-4,5-dimethylpyridazine (550 mg) in polyphosphoric acid (5 mL) was added ethyl 4-methyl-3-oxo-pentanoate (1.13 mL). The mixture was heated to 85° C. for 1 hour, then 120° C. for 1 hour. While still hot, the mixture was slowly added to a stirred solution of saturated aqueous NaHCO₃ (150 mL) and chloroform/IPA (4:1) (50 mL). Upon complete addition, the mixture was stirred for 10 minutes and the organic layer was isolated. The aqueous layer was further extracted with chloroform/IPA (4:1) (3×50 mL) and the organic layers were pooled, dried over MgSO₄, filtered, and concentrated. The crude product was purified by normal phase chromatography (0-65% EtOAc/hexanes). The column was then flushed with hexanes followed by 0-2% MeOH/DCM, to give the title compound (204 mg). ¹H NMR (400 MHz, CDCl₃) δ 6.47 (s, 1H), 3.63 (hept, J=6.8 Hz, 1H), 2.56 (d, J=1.0 Hz, 3H), 2.44 (d, J=1.0 Hz, 3H), 1.32 (s, 3H), 1.30 (s, 3H). ES-MS [M+1]⁺: 252.

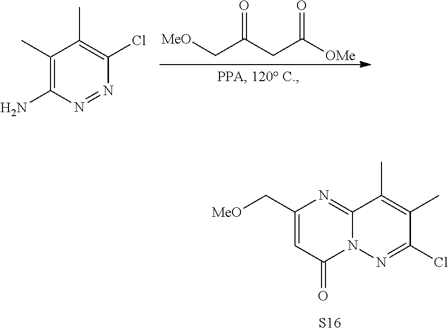

7-Chloro-2-(methoxymethyl)-8,9-dimethyl-4H-pyrimido[1,2-b]pyridazin-4-one (Intermediate S16): To a solution of 6-chloro-3-amino-4,5-dimethylpyridazine (1 g) in polyphosphoric acid (9 mL) was added methyl 4-methoxy-3-oxo-butanoate (1.23 mL). The mixture was heated to 85° C. for 1 hour, then 120° C. for 30 minutes. The mixture was then quenched by slow addition to a solution of saturated sodium bicarbonate (˜150 mL) and 4:1 chloroform/IPA (˜50 mL). The organic layer was isolated and the aqueous layer further extracted (×3). The organic layers were pooled, dried over magnesium sulfate, filtered, and concentrated. The crude product was dissolved in DMSO (9 mL) and solid was filtered off. The filtrate was purified using the reverse phase chromatography (5-55% ACN/0.05% aqueous NH₄OH). The fractions containing product were concentrated to give the title compound (219 mg) as a solid. The solid was washed with hexanes then dissolved in DCM and purified by normal phase chromatography (0-2% MeOH with 10% NH₄OH/DCM). The fractions containing product were concentrated to give the title compound (682 mg). ¹H NMR (400 MHz, CDCl₃) δ 6.77 (t, J=1.0 Hz, 1H), 4.45 (d, J=1.0 Hz, 2H), 3.51 (s, 3H), 2.59 (d, J=0.9 Hz, 3H), 2.49 (d, J=1.0 Hz, 3H). ES-MS [M+1]⁺: 254.

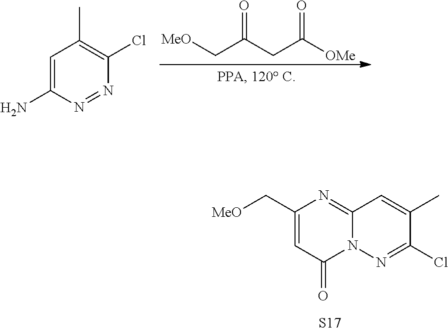

7-Chloro-2-(methoxymethyl)-8-methyl-4H-pyrimido[1,2-b]pyridazin-4-one (Intermediate S17): To a solution of 6-chloro-3-amino-5-methylpyridazine (500 mg) in polyphosphoric acid (5 mL) was added methyl 4-methoxy-3-oxo-butanoate (673 μL, 5.22 mmol). The mixture was heated to 85° C. for 1 hour, then 120° C. for 30 minutes. The mixture was then quenched by slow addition to a solution of saturated sodium bicarbonate (˜150 mL) and 4:1 chloroform/IPA (˜50 mL). The organic layer was isolated and the aqueous layer further extracted (3×). The organic layers were pooled, dried over magnesium sulfate, filtered, and concentrated. The crude product was dissolved in DMSO (12 mL) and purified by reverse-phase HPLC (0-45% ACN/0.05% aqueous NH₄OH). The fractions containing product were concentrated to give the title compound (690 mg). ¹H NMR (400 MHz, CDCl₃) δ 7.61 (q, J=1.3 Hz, 1H), 6.76 (d, J=1.1 Hz, 1H), 4.42 (s, 2H), 3.50 (s, 3H), 2.50 (d, J=1.2 Hz, 3H). ES-MS [M+1]⁺: 240.

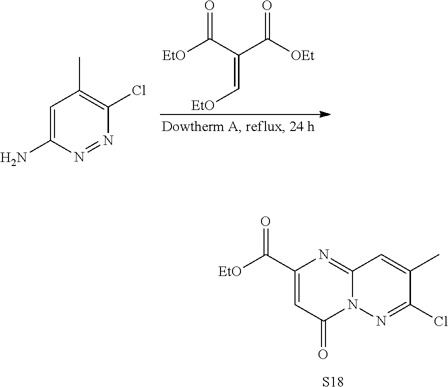

Ethyl 7-chloro-8-methyl-4-oxo-4H-pyrimido[1,2-b]pyridazine-2-carboxylate (Intermediate S18): A mixture of diethyl ethoxymethylenemalonate (641 μL) and 6-chloro-3-amino-5-methylpyridazine (500 mg) in Dowtherm™ A (6 mL) was heated to 200° C. for 18 hours. The reaction mixture was cooled to room temperature and purified via normal phase chromatography (0-55% EtOAc/DCM). The fractions containing desired product were concentrated and determined to be impure by LCMS. The material was dissolved in DMSO (9 mL) and purified by reverse-phase HPLC (5-50% ACN/0.05% aqueous NH₄OH). The fractions containing product were concentrated to give the title compound (250 mg). ¹H NMR (400 MHz, CDCl₃) δ 8.93 (s, 1H), 7.75 (s, 1H), 4.42 (q, J=7.1 Hz, 2H), 2.57 (d, J=0.9 Hz, 3H), 1.41 (t, J=7.1 Hz, 3H). ES-MS [M+1]⁺: 268.2.

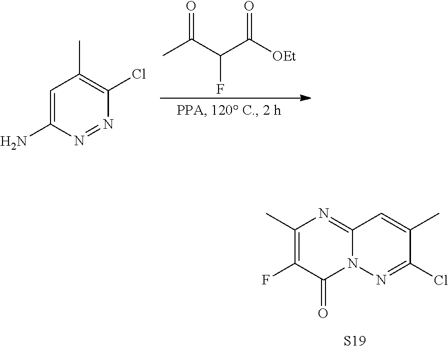

7-Chloro-3-fluoro-2,8-dimethyl-4H-pyrimido[1,2-b]pyridazin-4-one (Intermediate S19). To a solution of 6-chloro-3-amino-5-methylpyridazine (550 mg) in polyphosphoric acid (5.5 mL) was added ethyl 2-fluoroacetoacetate (961 μL). The mixture was heated to 120° C. for 2 hours. The mixture was then quenched by slow addition to a solution of saturated sodium bicarbonate (˜150 mL) and 4:1 chloroform/IPA (˜50 mL). Upon complete addition, the mixture was allowed to stir for 15 minutes and the organic layer was isolated. The aqueous layer was further extracted with chloroform:IPA (4:1) thrice. The crude product was dissolved in DMSO (12 mL) and purified by RP—HPLC (0-45% MeCN/0.05% aqueous NH₄OH). The fractions containing product were concentrated to give 627 mg of the title compound. ¹H NMR (400 MHz, CDCl₃) δ 7.60 (q, J=1.3 Hz, 1H), 2.52-2.47 (m, 6H). ES-MS [M+H]⁺=228.

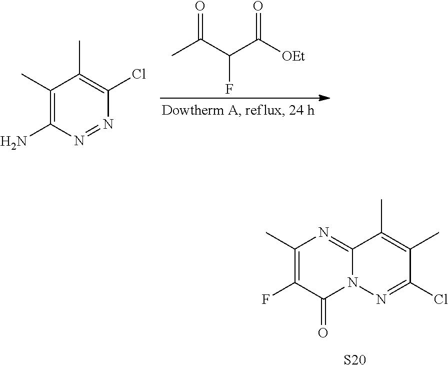

7-Chloro-3-fluoro-2,8,9-trimethyl-4H-pyrimido[1,2-b]pyridazin-4-one (Intermediate S20). To a solution of 6-chloro-3-amino-4,5-dimethylpyridazine (550 mg) in Dowtherm™ A (4 mL) was added ethyl 2-fluoro-3-oxo-butanoate (525 μL) and the reaction was heated to 150° C. for 18 h. Additional ethyl 2-fluoro-3-oxo-butanoate (525 μL) was added and the reaction stirred for an additional 18 h at 150° C. The reaction mixture was purified directly via flash column chromatography on silica gel (0-20% Hex/EtOAc; then 0-30% EtOAc/DCM) to afford 243 mg of the title compound. ¹H NMR (400 MHz, CDCl₃) δ 2.62 (d, J=0.9 Hz, 3H), 2.51 (d, J=3.6 Hz, 3H), 2.48 (t, J=0.9 Hz, 3H). ES-MS [M+1]⁺: 268.

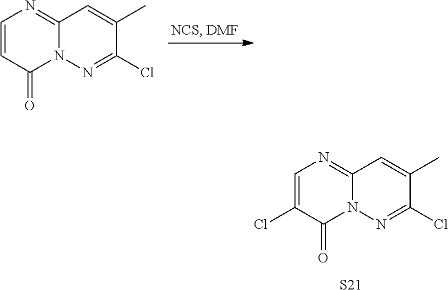

3,7-Dichloro-8-methyl-4H-pyrimido[1,2-b]pyridazin-4-one (Intermediate S21). To a solution of 7-chloro-8-methyl-4H-pyrimido[1,2-b]pyridazin-4-one (50 mg) in DMF (0.71 mL) was added N-chlorosuccinimide (36 mg). The mixture was stirred at room temperature for 72 h. Additional N-chlorosuccinimide (8 mg) was added and the mixture stirred for 48 h. The mixture was poured into water and extracted with DCM (3×). The combined organics were passed through a phase separator and concentrated to afford 42 mg of the title compound which was carried forward without further purification. ¹H NMR (400 MHz, DMSO-d₆) δ 7.76 (s, 1H), 7.31 (q, J=1.3 Hz, 1H), 1.63 (d, J=1.3 Hz, 3H); ES-MS [M+1]⁺: 230/232.

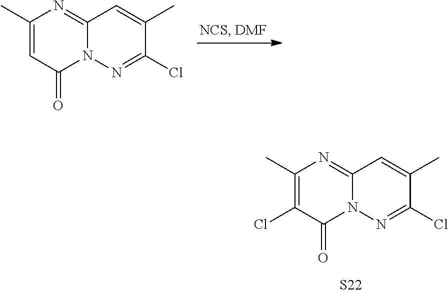

3,7-Dichloro-2,8-dimethyl-4H-pyrimido[1,2-b]pyridazin-4-one (Intermediate S22). To a solution of 7-chloro-2,8-dimethyl-4H-pyrimido[1,2-b]pyridazin-4-one (50 mg) in DMF (0.66 mL) was added N-chlorosuccinimide (33 mg). The mixture was stirred at room temperature for 18 h. Additional N-chlorosuccinimide (8 mg) was added and the mixture stirred for 48 h. The mixture was poured into water and extracted with DCM (3×). The combined organics were passed through a phase separator and concentrated. Purification via flash column chromatography (0-30% EtOAc/DCM) afforded 80 mg of the title compound. ¹H NMR (400 MHz, CDCl₃) δ 7.65 (q, J=1.3 Hz, 1H), 2.60 (s, 3H), 2.51 (d, J=1.3 Hz, 3H). ES-MS [M+1]⁺: 244.

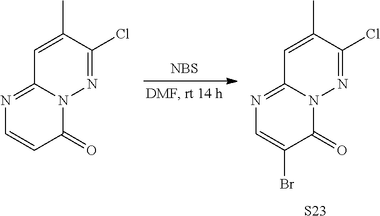

3-Bromo-7-chloro-8-methyl-4H-pyrimido[1,2-b]pyridazin-4-one (Intermediate S23) To a solution of 7-chloro-8-methyl-4H-pyrimido[1,2-b]pyridazin-4-one (150 mg, 0.77 mmol) in DMF (2.15 mL) was added N-bromosuccinimide (143 mg, 0.81 mmol). The mixture was stirred at room temperature for 14 h, then purified by reverse-phase chromatography (5-50% MeCN/0.05% NH₄OH). The fractions containing product were concentrated to give the title compound (175 mg). ES-MS [M+H]⁺=276.0.

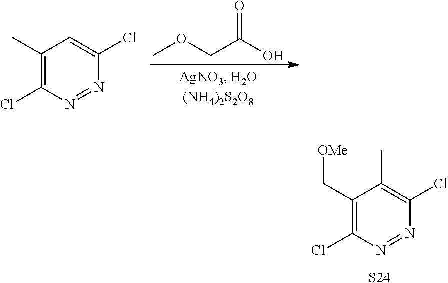

3,6-Dichloro-4-(methoxymethyl)-5-methylpyridazine (Intermediate S24): A stirred solution of 3,6-dichloro-4-methylpyridazine (2 g), methoxyacetic acid (2.82 mL) and silver nitrate (1.04 g) in water (100 mL) was heated to 72° C. where ammonium persulfate (4.2 g) was added slowly in several portions. The mixture was heated for 30 minutes at 72° C., then for 1 h at 90° C. Additional ammonium persulfate was added portionwise (2.4 g) and the reaction was allowed to stir for another 1 h at 90° C. The mixture was cooled to room temperature and slowly poured into a solution of saturated aqueous bicarbonate (˜150 mL) and chloroform/IPA (4:1) and stirred for 20 minutes, then extracted (3×). The organic layers were pooled, dried over magnesium sulfate, filtered, and concentrated. The crude product was dissolved in DMSO (15 mL) and purified by reverse-phase HPLC (15-60% ACN/0.1% aqueous TFA). The fractions containing product were basified with sat. NaHCO₃, extracted with 3:1 chloroform/IPA, dried over magnesium sulfate, filtered, and concentrated to give the title compound (1.6 g). ¹H NMR (400 MHz, CDCl₃) δ 4.64 (s, 2H), 3.44 (s, 3H), 2.52 (s, 3H). ES-MS [M+1]⁺: 208.

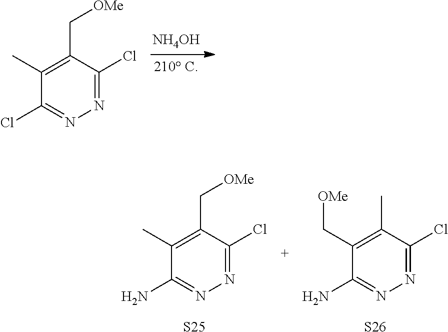

6-Chloro-5-(methoxymethyl)-4-methylpyridazin-3-amine (Intermediate S25): 3,6-Dichloro-4-(methoxymethyl)-5-methyl-pyridazine (1.59 g) was mixed with ammonium hydroxide (78 mL) solution and heated to 210° C. for 24 hours in a stainless steel pressure vessel. The reaction was cooled to room temperature and the reaction mixture was transferred to a round bottom flask with DCM/MeOH and concentrated. The crude product was dissolved in DMSO (12 mL) and purified by reverse-phase HPLC (0-35% ACN/0.05% aqueous NH₄OH) to afford the title compound (364 mg). ¹HNMR (400 MHz, CDCl₃) δ 4.72 (s, 2H), 4.59 (s, 2H), 3.42 (s, 3H), 2.23 (s, 3H). ES-MS [M+1]⁺: 188.

6-Chloro-4-(methoxymethyl)-5-methylpyridazin-3-amine (Intermediate S26): The intermediate was synthesized and isolated as described in the previous step (554 mg). ¹H NMR (400 MHz, CDCl₃) δ 5.27 (s, 2H), 4.52 (s, 2H), 3.38 (s, 3H), 2.36 (s, 3H). ES-MS [M+1]⁺: 188.

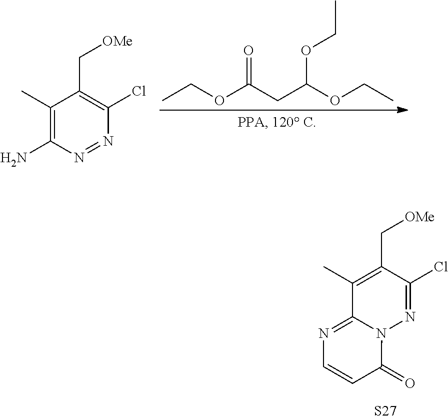

7-Chloro-8-(methoxymethyl)-9-methyl-4H-pyrimido[1,2-b]pyridazin-4-one (Intermediate S27): To a mixture of ethyl 3,3-diethoxypropionate (755 μL) and 6-chloro-5-(methoxymethyl)-4-methyl-pyridazin-3-amine (364 mg) was added polyphosphoric acid (3 mL). The mixture was stirred at 120° C. for 5 hours. More ethyl 3,3-diethoxypropionate (755 μL) was added and the mixture stirred at 120° C. for an additional 1 hour. The reaction mixture was then slowly added to a stirred solution of 100 mL of saturated aqueous NaHCO₃ and 50 mL of DCM. After complete addition, the mixture was allowed to stir for 20 minutes, and the organic layer was separated. The aqueous layer was further extracted with chloroform/IPA (4:1) (×3). The organic layers were combined, dried over MgSO₄, filtered, and concentrated. The crude product was dissolved in DMSO (12 mL) and purified by reverse-phase HPLC (0-55% ACN/0.05% aqueous NH₄OH). The fractions containing product were concentrated to give the title compound (108 mg). ¹H NMR (400 MHz, CDCl₃) δ 8.23 (d, J=6.4 Hz, 1H), 6.68 (d, J=6.4 Hz, 1H), 4.65 (s, 2H), 3.49 (s, 3H), 2.71 (s, 3H). ES-MS [M+1]⁺: 240.

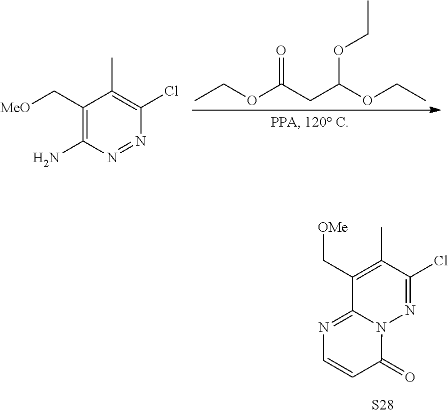

7-Chloro-9-(methoxymethyl)-8-methyl-4H-pyrimido[1,2-b]pyridazin-4-one (Intermediate S28): To a mixture of ethyl 3,3-diethoxypropionate (1.15 mL) and 6-chloro-4-(methoxymethyl)-5-methylpyridazin-3-amine (554 mg) was added polyphosphoric acid (5 mL). The mixture was stirred at 120° C. for 1 hour. More ethyl 3,3-diethoxypropionate (1.15 mL) was added and the mixture stirred at 120° C. for an additional 1 hour. The reaction mixture was then slowly added to a stirred solution of 150 mL of saturated aqueous NaHCO₃ and 100 mL of DCM. After complete addition, the mixture was allowed to stir for 20 minutes, and the organic layer was separated. The aqueous layer was further extracted with chloroform/IPA (4:1) (3×). The organic layers were combined, dried over MgSO₄, filtered, and concentrated. The crude product was dissolved in DMSO (12 mL) and purified by reverse-phase HPLC (0-40% ACN/0.05% aqueous NH₄OH). The fractions containing product were concentrated to give the title compound. ¹H NMR (400 MHz, CDCl₃) δ 8.22 (d, J=6.4 Hz, 1H), 6.64 (d, J=6.4 Hz, 1H), 4.95 (s, 2H), 3.48 (s, 3H), 2.58 (s, 3H). ES-MS [M+1]⁺: 240.

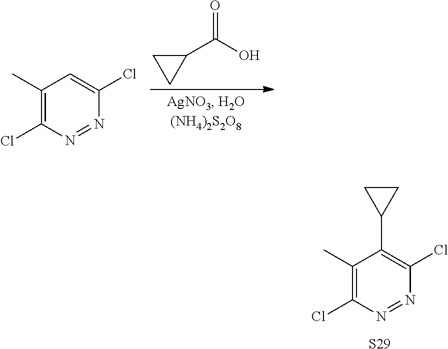

3,6-Dichloro-4-cyclopropyl-5-methylpyridazine (Intermediate S29): A stirred solution of 3,6-dichloro-4-methylpyridazine (2.5 g), cyclopropanecarboxylic acid (3.66 mL) and silver nitrate (1.3 g) in water (125 mL) was heated to 72° C. where ammonium persulfate (5.25 g) was added slowly in several portions. The mixture was heated for 20 minutes at 72° C. Sulfuric acid (1.23 mL) was added, and the mixture was heated to 90° C. for 1 hour. The mixture was cooled to ambient temperature and slowly poured into a solution of saturated aqueous bicarbonate (˜150 mL) and chloroform/IPA (4:1) (˜75 mL) and stirred for 20 minutes then extracted with chloroform/IPA (4:1) (3×). The organic layerswere combined, dried over magnesium sulfate, filtered, and concentrated. The crude product was purified by normal phase chromatography (0-25% EtOAc/hexanes) to afford the title compound (1.95 g). ¹H NMR (400 MHz, CDCl₃) δ 2.54 (d, J=0.9 Hz, 3H), 1.88-1.75 (m, 1H), 1.34-1.19 (m, 2H), 0.81-0.67 (m, 2H). ES-MS [M+1]⁺: 203.

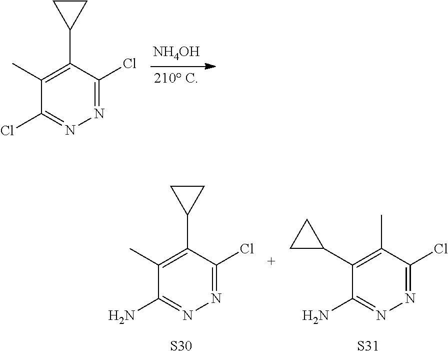

6-Chloro-5-cyclopropyl-4-methylpyridazin-3-amine (Intermediate S30) and 6-chloro-4-cyclopropyl-5-methylpyridazin-3-amine (Intermediate S31): 3,6-Dichloro-4-cyclopropyl-5-methyl-pyridazine (1.0 g) was mixed with ammonium hydroxide (15 mL; 28% NH₃ in water) solution and 1,4-dioxane (2 mL). The solution was heated at 210° C. for 24 hours (140 psi). The reaction was cooled to room temperature and the reaction mixture was transferred to a round bottom flask with DCM/MeOH and concentrated. The crude product was purified by normal phase chromatography (0-90% EtOAc/DCM; then 0-10% MeOH/DCM/NH₄OH) to give a mixture of the title compounds. ¹H NMR (400 MHz, CDCl₃) δ 2.46 (d, J=1.2 Hz, 3H), 1.90-1.79 (m, 1H), 1.42-1.29 (m, 2H), 0.86-0.73 (m, 2H). ¹H NMR (400 MHz, CDCl₃) δ 2.60 (d, J=1.3 Hz, 3H), 1.80-1.55 (m, 1H), 1.44-1.32 (m, 2H), 0.81-0.72 (m, 2H). ES-MS

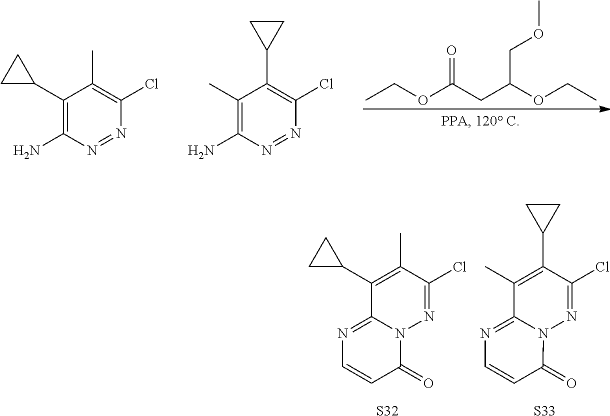

7-Chloro-9-cyclopropyl-8-methyl-4H-pyrimido[1,2-b]pyridazin-4-one (Intermediate S32): To a mixture of isomers 6-chloro-5-cyclopropyl-4-methyl-pyridazin-3-amine and 6-chloro-4-cyclopropyl-5-methyl-pyridazin-3-amine (928 mg), was added ethyl 3,3-diethoxypropionate (1.97 mL) and polyphosphoric acid (7 mL). The mixture was stirred at 120° C. for 1 hour. More ethyl 3,3-diethoxypropionate (1.97 mL) was added, and the mixture was stirred an additional 1 hour at 120° C. The reaction mixture was then slowly added to a stirred solution of 150 mL of saturated aqueous NaHCO₃ and 100 mL of DCM. After complete addition, the mixture was allowed to stir for 20 minutes, and the organic layer was separated. The aqueous layer was further extracted with chloroform/IPA (4:1) (3×). The organic layers were combined, dried over MgSO₄, filtered, and concentrated. The crude product was dissolved in DMSO (15 mL) and purified by reverse-phase HPLC (0-45% ACN/0.1% aqueous TFA). The fractions containing product were neutralized with sat. NaHCO₃ and extracted with 3:1 chloroform/IPA. The organic layers were concentrated to give the title compound (73 mg). ES-MS [M+1]⁺: 236.

7-Chloro-8-cyclopropyl-9-methyl-4H-pyrimido[1,2-b]pyridazin-4-one (Intermediate S33): Intermediate S33 was synthesized and isolated as described in the previous step to give the title compound (473 mg). ES-MS [M+1]⁺: 236.

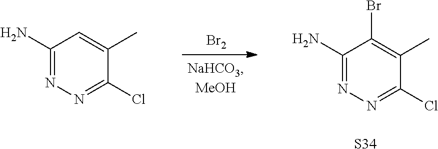

4-Bromo-6-chloro-5-methylpyridazin-3-amine (Intermediate S34): A mixture of 6-chloro-5-methylpyridazin-3-amine (4 g) and NaHCO₃ (4.68 g) were suspended in MeOH (40 mL) and treated with Br₂ (6.68 g). The mixture was stirred at 20° C. for 4 hours. The mixture was filtered and the cake was washed with a mixture of DCM/MeOH (10:1) (100 mL). The filtrate was concentrated. The residue was diluted with saturated Na₂SO₃ aqueous solution (50 mL), then extracted by DCM (100 mL×5). The combined organic layers were dried over Na₂SO₄, and concentrated to give 4-bromo-6-chloro-5-methylpyridazin-3-amine (6.07 g). ¹H NMR (400 MHz, CDCl₃) δ 5.86 (br s, 2H), 2.52 (s, 3H).

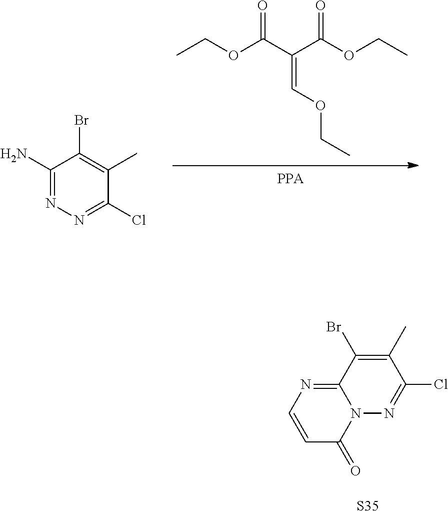

9-Bromo-7-chloro-8-methyl-4H-pyrimido[1,2-b]pyridazin-4-one (Intermediate S35): A mixture of 4-bromo-6-chloro-5-methylpyridazin-3-amine (6 g) and diethyl 2-(ethoxymethylene)malonate (11.66 g) in PPA (100 mL) was stirred at 120° C. for 2 hours. The mixture was added to sat. aq. Na₂CO₃, while maintaining a pH of about 8, and then extracted with EtOAc (50 mL×4). The combined organic layers were washed with brine (20 mL×2), dried over Na₂SO₄, filtered, and concentrated. The residue was purified by Combi Flash (0 to 70%, ethyl acetate in petroleum ether) to give 9-bromo-7-chloro-8-methyl-4H-pyrimido[1,2-b]pyridazin-4-one (4.1 g). ¹H NMR (400 MHz, CDCl₃) δ 8.28 (d, J=6.8 Hz, 1H), 6.68 (d, J=6.4 Hz, 1H), 2.73 (s, 3H).

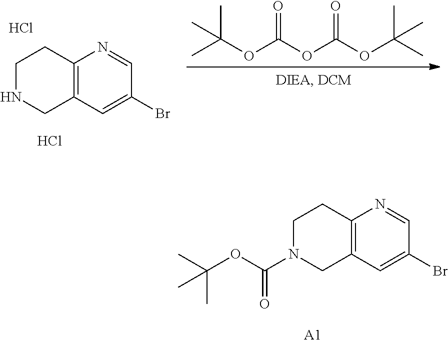 [SMILES: Cl ; Brc1cnc2c(c1)CNCC2 ; CC(C)(C)OC(=O)OC(=O)OC(C)(C)C ; Cl ; CC(C)(C)OC(=O)N1CCc2ncc(Br)cc2C1 ; "A1"]

tert-Butyl 3-bromo-7,8-dihydro-1,6-naphthyridine-6(5H)-carboxylate (Intermediate A1). To a suspension of 3-bromo-5,6,7,8-tetrahydro-1,6-naphthyridine dihydrochloride (10 g) in DCM (150 mL) at ambient temperature was added di-tert-butyl dicarbonate (12 mL) followed by slow addition of DIEA (24.4 mL). After stirring for 18 hat room temperature, the mixture was concentrated under reduced pressure. Purification using normal phase chromatography (0-50% EtOAc/hexanes) provided 10.7 g of the title compound. ¹H NMR (400 MHz, CDCl₃) δ 8.49 (d, J=2.2 Hz, 1H), 7.60 (s, 1H), 4.59 (s, 2H), 3.74 (t, J=6.0 Hz, 2H), 2.98 (t, J=6.0 Hz, 2H), 1.49 (s, 9H); ES-MS [M+1]⁺: 313.3/315.3.

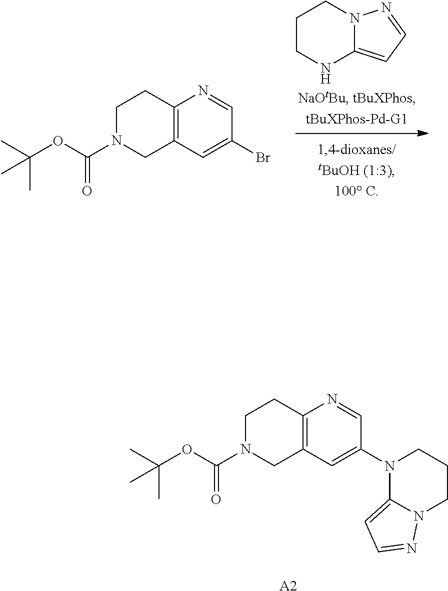 [SMILES: c1cc2n(n1)CCCN2 ; CC(C)(C)OC(=O)N1CCc2ncc(Br)cc2C1 ; CC(C)(C)OC(=O)N1CCc2ncc(N3CCCn4nccc43)cc2C1 ; "A2"]

tert-Butyl 3-(6,7-dihydropyrazolo[1,5-a]pyrimidin-4(5H)-yl)-7,8-dihydro-1,6-naphthyridine-6(5H)-carboxylate (Intermediate A2). Divided equally amoungst three microwave vials was added a solution of tert-butyl 3-bromo-7,8-dihydro-1,6-naphthyridine-6(5H)-carboxylate (2.82 g), 4,5,6,7-tetrahydropyrazolo[1,5-a]pyrimidine (1.66 g), NaO^{t}Bu (1.73 g), tBuXPhos (382 mg), and tBuXPhos-Pd-G1 (619 mg) in 1,4-dioxanes (15 mL) and t-BuOH (45 mL). The solutions were purged with nitrogen and stirred at 100° C. for 4 h. After cooling to ambient temperature, the mixtures were filtered over a pad of Celite® which was washed thouroughly with EtOAc/DCM. The filtrate was concentrated in vacuo, and the resulting residue was dissolved in DMSO/DMF (1:1) (36 mL). After passing through a syringe filter, purification via RP-HPLC (10-50% MeCN/0.1% aqueous TFA) afforded the title compound (3.71 g). ¹H NMR (400 MHz, DMSO-d₆) δ 8.49 (d, J=2.6 Hz, 1H), 7.86 (d, J=2.6 Hz, 1H), 7.30 (d, J=2.1 Hz, 1H), 5.80 (d, J=2.1 Hz, 1H), 4.61 (s, 2H), 4.13 (t, J=6.2 Hz, 2H), 3.91-3.70 (m, 2H), 3.67 (t, J=5.9 Hz, 2H), 2.91 (t, J=5.9 Hz, 2H), 2.21 (p, J=6.1 Hz, 2H), 1.43 (s, 9H); ES-MS [M+1-TFA]⁺: 356.5.

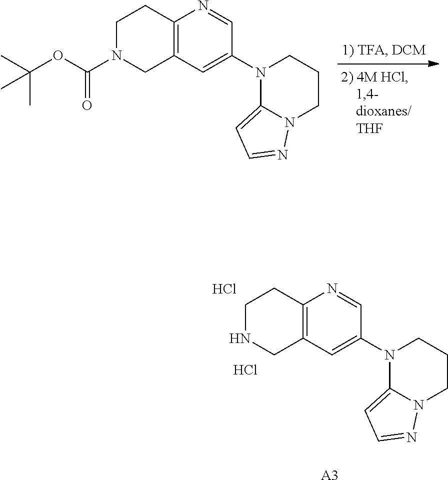 [SMILES: CC(C)(C)OC(=O)N1CCc2ncc(N3CCCn4nccc43)cc2C1 ; Cl ; Cl ; c1cc2n(n1)CCCN2c1cnc2c(c1)CNCC2 ; "A3"]

3-(6,7-Dihydropyrazolo[1,5-a]pyrimidin-4(5H)-yl)-5,6,7,8-tetrahydro-1,6-naphthyridine dihydrochloride (Intermediate A3). To a solution of tert-butyl 3-(6,7-dihydropyrazolo[1,5-a]pyrimidin-4(5H)-yl)-7,8-dihydro-1,6-naphthyridine-6(5H)-carboxylate (3.72 g) in DCM (50 mL) was added TFA (10 mL) and the solution was allowed to stir at ambient temperature for 2 h. The reaction mixture was concentrated in vacuo, and the crude material was suspended in THF (10 mL). A 4M HCl solution in 1,4-dioxanes (20 mL) was added and the mixture was allowed to stir for 30 minutes and then concentrated in vacuo. The residue was then azeotroped with MeOH (3×) to remove excess solvent. The solid was dried in a vacuum oven to provide 1.85 g of the title compound, which was taken forward without further purification. ES-MS [M+1−2·HCl]⁺: 256.2.

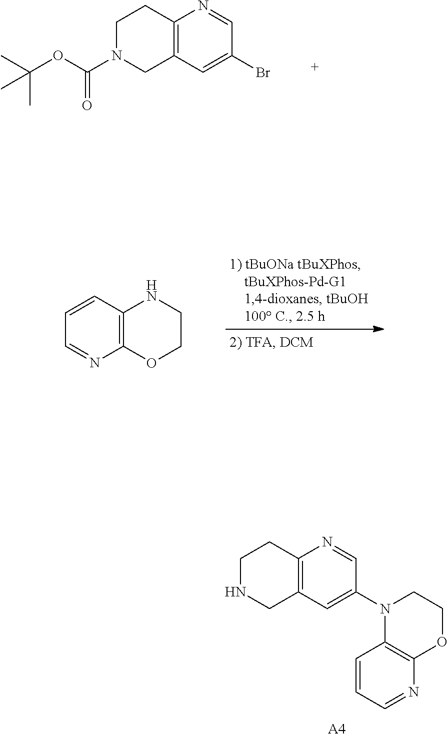

1-(5,6,7,8-Tetrahydro-1,6-naphthyridin-3-yl)-2,3-dihydro-1H-pyrido[2,3-b][1,4]oxazine (Intermediate A4). tert-Butyl 3-bromo-7,8-dihydro-5H-1,6-naphthyridine-6-carboxylate (2.82 g, 9.0 mmol), 8-azabenzomorpholine (1112193-37-9) (1.47 g, 10.8 mmol), sodium tert-butoxide (1.73 g, 18 mmol), t-BuXPhos Palladacycle Gen 1 (928 mg, 1.35 mmol), t-BuXPhos (573 mg, 1.35 mmol), 1,4-dioxane (9.0 mL) and t-BuOH (27 mL) were combined in a vial and degassed (3×). The reaction was heated at 100° C. for 2.5 h. The reaction was filtered over Celite® rinsed with DCM, and concentrated. The crude oil was purified by reverse-phase chromatography (5-45% MeCN/Water/0.1% aqueous TFA). The desired fractions were concentrated, and the residue was dissolved in DCM (40 mL) and TFA (2.07 mL). After 2 h, the solvents were removed, diluted with MeOH, and loaded onto a SCX-cartridge (HF bond). The cartridge was washed with MeOH and eluted with 7N NH₃ in MeOH to produce the title compound (1.2 g). ES-MS [M+H]⁺=269.5.

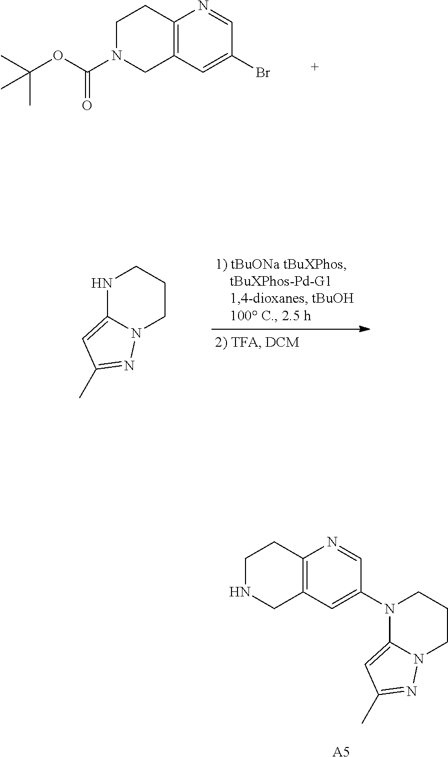

3-(2-Methyl-6,7-dihydropyrazolo[1,5-a]pyrimidin-4(5H)-yl)-5,6,7,8-tetrahydro-1,6-naphthyridine (Intermediate A5). Intermediate A5 was prepared in a similar manner as Intermediate A4. ES-MS [M+H]⁺=270.4.

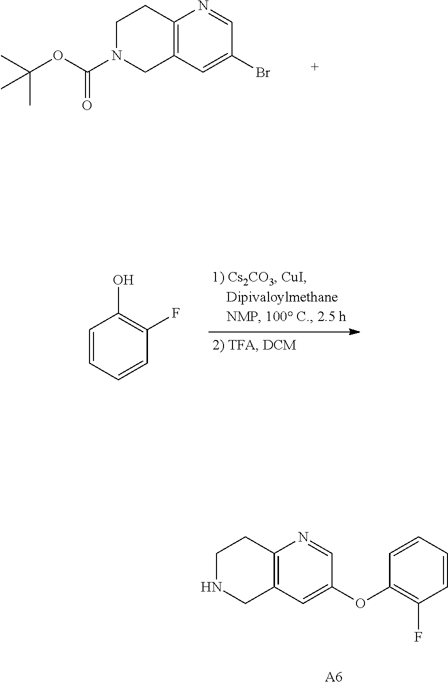 [SMILES: CC(C)(C)OC(=O)N1CCc2ncc(Br)cc2C1 ; Oc1ccccc1F ; Fc1ccccc1Oc1cnc2c(c1)CNCC2 ; "A6"]

3-(2-Fluoro phenoxy)-5,6,7,8-tetrahydro-1,6-naphthyridine (Intermediate A6). To a microwave vial were added tert-butyl 3-bromo-7,8-dihydro-5H-1,6-naphthyridine-6-carboxylate (3.0 g, 9.58 mmol), 2-fluorophenol (367-12-4) (2.15 g, 19.2 mmol), cesium carbonate (534-17-8) (6.28 g, 19.2 mmol), Dipivaloylmethane (200 μL, 0.96 mmol), and copper(I) iodide (91.2 mg, 0.48 mmol). The solids were degassed followed by addition of NMP (48 mL). The resulting solution was stirred at 140° C. for 18 h. The reaction was filtered over Celite®, washed with DCM and concentrated. The resulting solution was passed through a syringe filter and purified by reverse-phase HPLC (5-45% MeCN/0.1% aqueous TFA). The desired fractions were concentrated, and the residue was dissolved in DCM (48 mL) and TFA (7.34 mL, 95.8 mmol). After 1 h, the reaction was concentrated, and purified by SCX cartridge (10G), washed with MeOH, and eluted with 7N NH₃/MeOH to produce the title compound (950 mg). ES-MS [M+H]⁺=245.4.

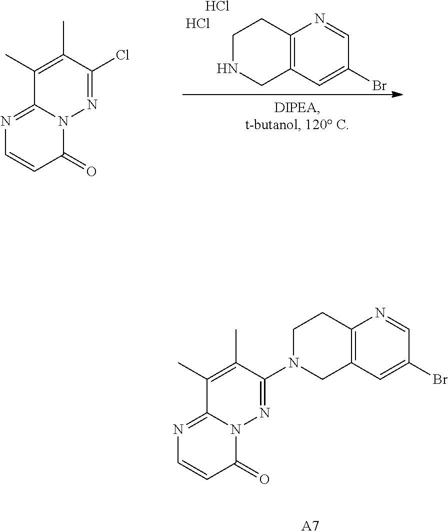 [SMILES: Cl ; Cc1c(Cl)nn2c(=O)ccnc2c1C ; Cl ; Brc1cnc2c(c1)CNCC2 ; Cc1c(N2CCc3ncc(Br)cc3C2)nn2c(=O)ccnc2c1C ; "A7"]

7-(3-Bromo-7,8-dihydro-1,6-naphthyridin-6(5H)-yl)-8,9-dimethyl-4H-pyrimido[1,2-b]pyridazin-4-one (Intermediate A7). 7-Chloro-8,9-dimethyl-4H-pyrimido[1,2-b]pyridazin-4-one (415 mg, 1.98 mmol), N,N-diisopropylethylamine (1.72 mL, 9.89 mmol), 7-bromo-2,5-diazatetralin dihydrochloride (679 mg, 2.37 mmol), and tert-butanol (9.90 mL) were combined and heated at 120° C. for 18 h. The solvents were removed, and the crude material was crystallized from MeOH at −78° C. to provide the first batch of the title compound (450 mg). The remaining filtrate was purified using reverse phase HPLC to yield an additional batch of material which was combined to afford the title compound (515 mg). ES-MS [M+1]⁺: 386.2/388.2.

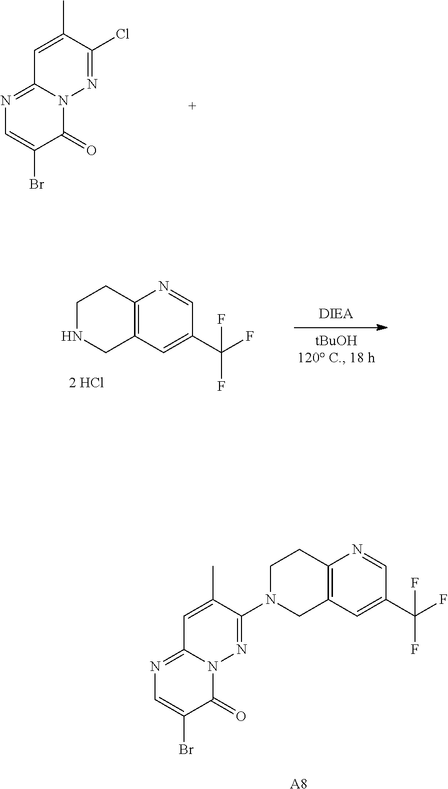 [SMILES: Cc1cc2ncc(Br)c(=O)n2nc1Cl ; FC(F)(F)c1cnc2c(c1)CNCC2 ; Cc1cc2ncc(Br)c(=O)n2nc1N1CCc2ncc(C(F)(F)F)cc2C1 ; "A8"]

3-Bromo-8-methyl-7-(3-(trifluoromethyl)-7,8-dihydro-1,6-naphthyridin-6(5H)-yl)-4H-pyrimido[1,2-b]pyridazin-4-one (Intermediate A8). To a solution of 3-(trifluoromethyl)-5,6,7,8-tetrahydro-1,6-naphthyridine dihydrochloride (CAS #870483-68-4) (150 mg, 0.55 mmol) in tert-butanol (3.6 mL) and NMP (3 mL) was added DIEA (0.19 mL, 1.09 mmol) and 3-bromo-7-chloro-8-methyl-pyrimido[1,2-b]pyridazin-4-one (100 mg, 0.36 mmol). The mixture was heated to 120° C. for 18 h. Solvents were removed, and the crude product was dissolved in DMSO (2 mL) and purified by reverse-phase HPLC (MeCN/0.1% aqueous TFA) to afford the title compound (100 mg). ES-MS [M+H]⁺=442.0.

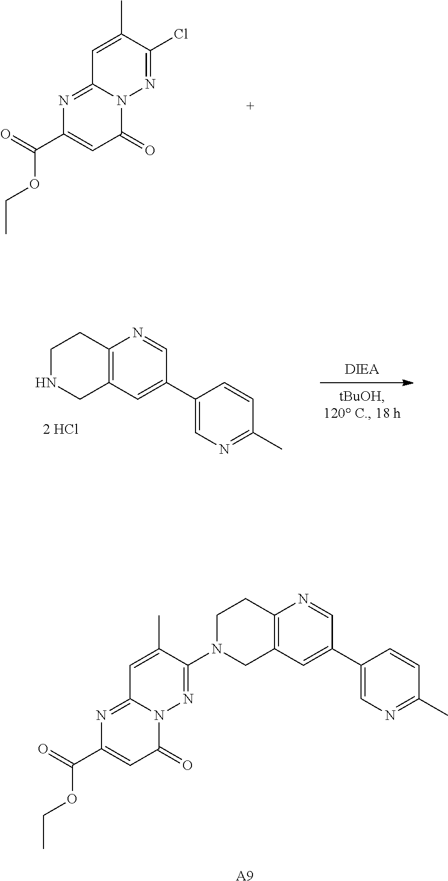 [SMILES: CCOC(=O)c1cc(=O)n2nc(Cl)c(C)cc2n1 ; Cc1ccc(-c2cnc3c(c2)CNCC3)cn1 ; CCOC(=O)c1cc(=O)n2nc(N3CCc4ncc(-c5ccc(C)nc5)cc4C3)c(C)cc2n1 ; "A9"]

Ethyl 8-methyl-7-(3-(6-methylpyridin-3-yl)-7,8-dihydro-1,6-naphthyridin-6(5H)-yl)-4-oxo-4H-pyrimido[1,2-b]pyridazine-2-carboxylate (Intermediate A9). To a solution of 3-(6-methyl-3-pyridyl)-5,6,7,8-tetrahydro-1,6-naphthyridine dihydrochloride (167.0 mg, 0.56 mmol) in tert-butanol (1.9 mL) was added DIEA (325 μL, 1.87 mmol) and ethyl 7-chloro-8-methyl-4-oxo-pyrimido[1,2-b]pyridazine-2-carboxylate (100 mg, 0.37 mmol). The mixture was heated to 120° C. for 18 h. Solvents were removed, and the crude product was dissolved in DMSO (5 mL) and purified by reverse-phase HPLC (MeCN/0.1% aqueous TFA) to afford the title compound (65 mg). ES-MS [M+H]⁺=457.2.

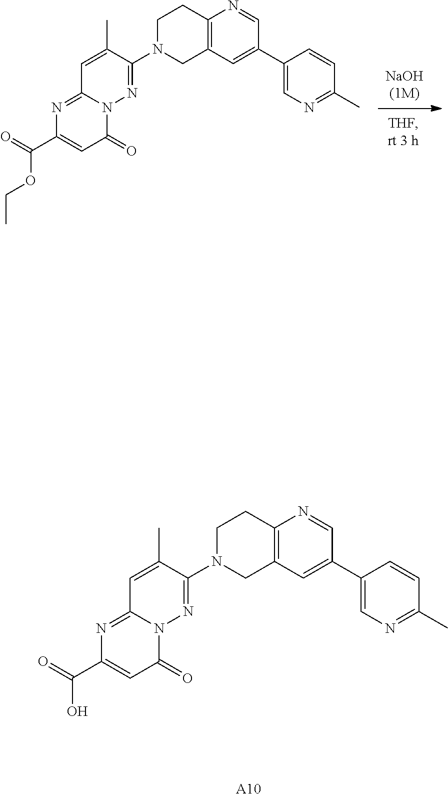 [SMILES: CCOC(=O)c1cc(=O)n2nc(N3CCc4ncc(-c5ccc(C)nc5)cc4C3)c(C)cc2n1 ; Cc1ccc(-c2cnc3c(c2)CN(c2nn4c(=O)cc(C(=O)O)nc4cc2C)CC3)cn1 ; "A10"]

8-Methyl-7-(3-(6-methylpyridin-3-yl)-7,8-dihydro-1,6-naphthyridin-6(5H)-yl)-4-oxo-4H-pyrimido[1,2-b]pyridazine-2-carboxylic acid (Intermediate A10). To a solution of 1M sodium hydroxide (0.75 mL, 0.75 mmol) in THF (0.75 mL) was added ethyl 8-methyl-7-[3-(6-methyl-3-pyridyl)-7,8-dihydro-5H-1,6-naphthyridin-6-yl]-4-oxo-pyrimido[1,2-b]pyridazine-2-carboxylate (69 mg, 0.15 mmol). After 3 h at rt, the reaction mixture was cooled to 0° C. and diluted with water (1 mL). To the mixture was added 1M HCl to pH 5-6 and the mixture was dried overnight to afford the title compound. ES-MS [M+H]⁺=429.4.

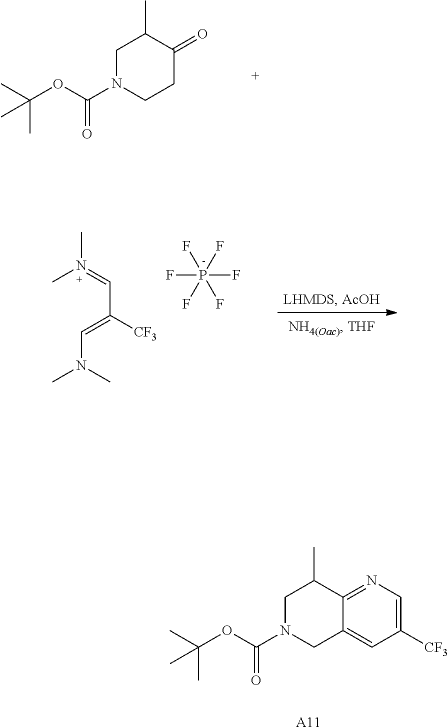

tert-Butyl 8-methyl-3-(trifluoromethyl)-7,8-dihydro-5H-1,6-naphthyridine-6-carboxylate (Intermediate A11). To a −15° C. solution of N-Boc-3-methyl-4-piperidone (400 mg) in THF (8 mL) was added dropwise a solution of 1M lithium bis(trimethylsilyl)amide (1.88 mL). The reaction warmed to room temperature for 1.5 h and was then added to a suspension of [(Z)-3-(dimethylamino)-2-(trifluoromethyl)prop-2-enylidene]-dimethyl-ammonium hexafluorophosphate (638 mg) in THF (5 mL) at −15° C. The resulting mixture was stirred at −15° C. for 2 h, then acetic acid (0.161 mL) was added, and the mixture was warmed to room temperature. After 1 h, ammonium acetate (413 mg) was added, and the mixture was heated at 65° C. for 2 h. The mixture was cooled, diluted with water, and extracted with diethyl ether. The organic layer was washed with brine, dried (Na₂SO₄), filtered, and concentrated. The crude residue was purified by normal-phase chromatography on silica gel (0-50% EtOAc/hexanes) to afford the title compound (60 mg). ES-MS [M+1]⁺: 317.2.

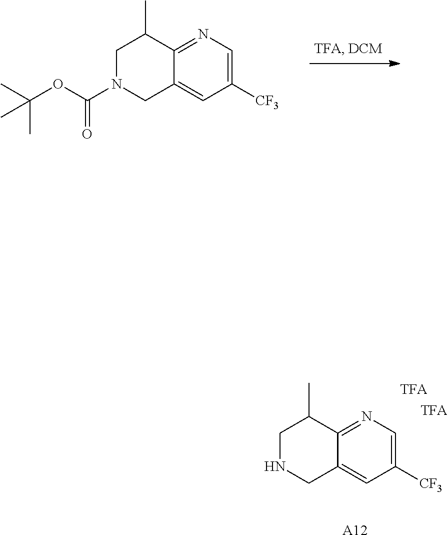 [SMILES: CC1CN(C(=O)OC(C)(C)C)Cc2cc(C(F)(F)F)cnc21 ; CC1CNCc2cc(C(F)(F)F)cnc21 ; "A12"]

8-Methyl-3-(trifluoromethyl)-5,6,7,8-tetrahydro-1,6-naphthyridine; 2,2,2-trifluoroacetic acid (Intermediate A12). To a vial were added tert-butyl 8-methyl-3-(trifluoromethyl)-7,8-dihydro-5H-1,6-naphthyridine-6-carboxylate (60 mg), DCM (0.9 mL), and trifluoroacetic acid (0.15 mL). The reaction stirred 1 hr and was concentrated to afford the title compound (75 mg). ES-MS [M+1]⁺: 217.2.

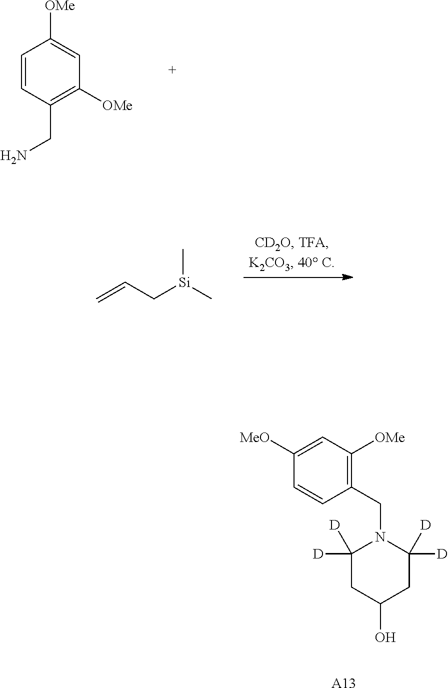

1-(2,4-Dimethoxybenzyl)piperidin-2,2,6,6-d₄-4-ol (Intermediate A13). Deuterated formaldehyde (7.58 mL, 55.0 mmol) was added to 2,4-dimethoxybenzylamine (3.64 mL, 23.9 mmol). Then, trifluoroacetic acid (1.83 mL, 23.9 mmol) was added. The resulting mixture was sonicated for 10 min and then stirred for 1 h at room temperature. To the resulting solution was added allyltrimethylsilane (4.18 mL, 26.3 mmol) and the reaction was heated at 40° C. for 18 h. The reaction was diluted with water (8 mL) and DCM (8 mL), and solid potassium carbonate (1.67 g, 12.0 mmol) was added. The mixture was stirred for 10 min and was then extracted with 3:1 CHCl₃/IPA (5×). The combined organic layers were dried (MgSO₄), filtered, and concentrated. The crude oil was purified by silica gel chromatography (0-20% MeOH/DCM) to afford the title compound. ¹H NMR (400 MHz, MeOD) δ 7.22 (d, J=8.6 Hz, 1H), 6.58 (d, J=2.3, 1H), 6.53 (dd, J=8.3, 2.4 Hz, 1H), 3.84 (s, 3H), 3.83-3.80 (m, 5H), 3.35 (s, 1H), 1.89 (dd, J=13.7, 3.7 Hz, 2H), 1.65 (dd, J=13.6, 8.1 Hz, 2H); ES-MS [M+1]⁺: 256.2.

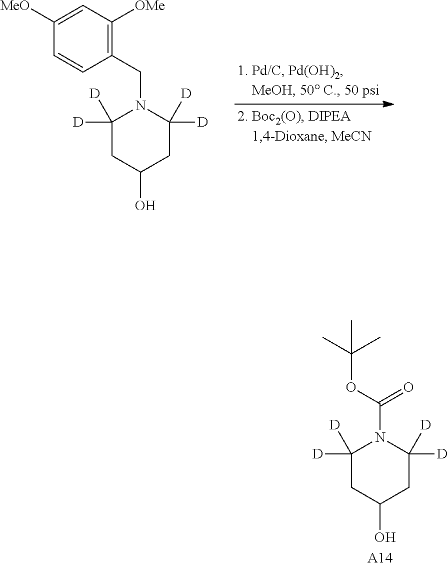

tert-Butyl 4-hydroxypiperidine-1-carboxylate-2,2,6,6-d₄ (Intermediate A14). To a degassed solution of 1-(2,4-dimethoxybenzyl)piperidin-2,2,6,6-d₄₋₄-ol (3.5 g, 13.7 mmol) in methanol (300 mL) was added palladium hydroxide (0.29 g, 2.1 mmol) and 10% palladium on activated carbon (0.22 g, 2.1 mmol). The reaction was charged with H₂ and stirred at 50° C. under 50 psi for 48 h. The reaction mixture was filtered over Celite®, washed with methanol, and concentrated under reduced pressure. The solid was combined with 1,4-dioxane (45 mL), acetonitrile (45 mL), and N,N-diisopropylethylamine (2.9 mL, 16.4 mmol). To the solution was added di-tert-butyl dicarbonate (4.7 mL, 20.5 mmol) and the reaction stirred at room temperature. After 3 h, the reaction was concentrated, and the crude oil was purified by normal-phase chromatography (0-20% MeOH/DCM) to afford the title compound (2.18 g). ¹H NMR (400 MHz, MeOD) δ 3.79-3.71 (m, 1H), 1.79 (dd, J=13.1, 3.8 Hz, 2H), 1.45 (s, 9H), 1.39-1.34 (m, 2H).

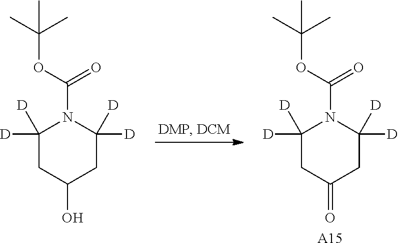

tert-Butyl 4-oxopiperidine-1-carboxylate-2,2,6,6-d₄ (Intermediate A15). To a solution of tert-butyl 4-hydroxypiperidine-1-carboxylate-2,2,6,6-d₄ (2.18 g, 10.6 mmol) in DCM (30 mL) was added Dess-Martin periodinane (6.75 g, 15.9 mmol). The reaction stirred atroom temperature for 18 h. The reaction was concentrated over Celite® and purified by normal-phase chromatography (0-50% EtOAc/Hexanes) to afford the title compound (1.69 g). ¹H NMR (400 MHz, CDCl₃) δ 2.42 (s, 4H), 1.49 (s, 9H).

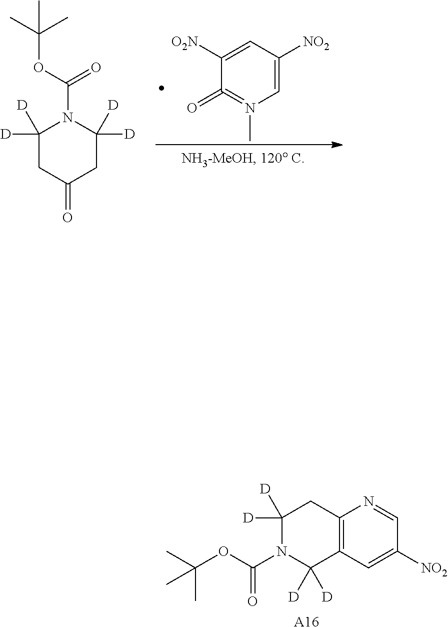

tert-Butyl 3-methyl-7,8-dihydro-1,6-naphthyridine-6(5H)-carboxylate-5,5,7,7-d₄ (Intermediate A16). In four separate microwave vials were combined equal portions of 1-methyl-3,5-dinitro-2-pyridone (1.0 g, 5.1 mmol), tert-butyl 4-oxopiperidine-1-carboxylate-2,2,6,6-d₄ (1.0 g, 5.1 mmol), and 2M ammonia-methanol solution (20.2 mL). The mixture was heated at 120° C. for 20 min in the microwave reactor. The reaction was concentrated over Celite® and purified by normal-phase chromatography (0-30% EtOAc/Hexanes) to afford the title compound (1.23 g). ¹H NMR (400 MHz, CDCl3) δ 9.25 (d, J=2.5 Hz, 1H), 8.23 (d, J=2.5, 1H), 3.11 (s, 2H), 1.50 (s, 9H); ES-MS [M+1]⁺: 284.1.

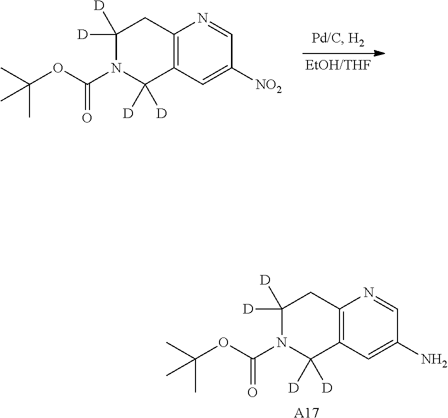

tert-Butyl 3-amino-7,8-dihydro-1,6-naphthyridine-6(5H)-carboxylate-5,5,7,7-d₄ (Intermediate A17). To a solution of tert-butyl 3-methyl-7,8-dihydro-1,6-naphthyridine-6(5H)-carboxylate-5,5,7,7-d₄ (1.23 g, 4.3 mmol) in ethanol (10 mL) and THF (10 mL) was added 10% palladium on activated carbon (527 mg, 4.9 mmol). The mixture was degassed and placed under H₂ balloon at 1 atm for 3 h. The reaction was filtered over Celite®, washed with EtOH, and the filtrate was concentrated to afford the title compound (1.03 g). ¹H NMR (400 MHz, CDCl₃) δ 7.94 (d, J=2.6 Hz, 1H), 6.73 (d, J=2.6, 1H), 3.60 (s, 2H), 2.87 (s, 2H), 1.48 (s, 9H); ES-MS [M+1]⁺: 254.1.

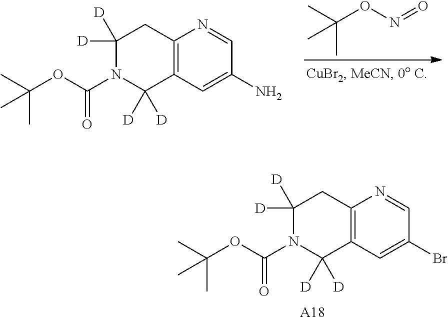

tert-Butyl 3-bromo-7,8-dihydro-1,6-naphthyridine-6(5H)-carboxylate-5,5,7,7-d₄ (Intermediate A18). Cupric bromide (767 mg, 3.4 mmol) was added to a solution of tert-butyl 3-amino-7,8-dihydro-1,6-naphthyridine-6(5H)-carboxylate-5,5,7,7-d₄ (580 mg, 2.3 mmol) in MeCN (8 mL). The reaction was cooled to 0° C. followed by dropwise addition of tert-butyl nitrite (0.33 mL, 2.8 mmol). The reaction stirred at 0° C. for 1 h and then room temperature for 5 h. The mixture was diluted with water and CHCl₃/IPA. The layers were separated and the aqueous layer was re-extracted with CHCl₃/IPA (2×). The combined organic phases were washed with brine (2×), dried (MgSO₄), filtered, and concentrated. The crude oil was purified by normal-phase chromatography (0-40% EtOAc/Hexanes) to afford the title compound (520 mg). ¹H NMR (400 MHz, CDCl₃) δ 8.47 (d, J=2.2 Hz, 1H), 7.56 (d, J=2.2, 1H), 2.93 (s, 2H), 1.48 (s, 9H); ES-MS [M+1]⁺: 317.1/319.1.

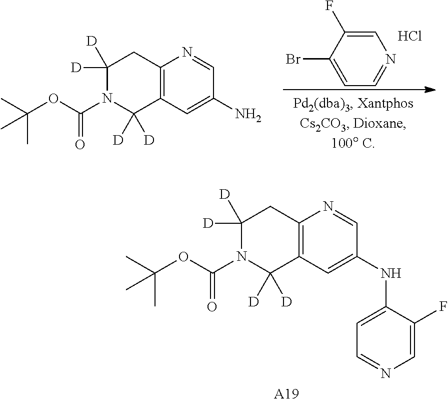 [SMILES: [2H]C1([2H])Cc2ncc(N)cc2C([2H])([2H])N1C(=O)OC(C)(C)C ; Fc1cnccc1Br ; Cl ; [2H]C1([2H])Cc2ncc(Nc3ccncc3F)cc2C([2H])([2H])N1C(=O)OC(C)(C)C ; "A19"]

tert-Butyl 3-((3-fluoropyridin-4-yl)amino)-7,8-dihydro-1,6-naphthyridine-6(5H)-carboxylate-5,5,7,7-d₄ (Intermediate A19). In a vial were combined 4-bromo-3-fluoropyridine hydrochloride (415 mg, 1.9 mmol), tert-butyl 3-amino-7,8-dihydro-1,6-naphthyridine-6(5H)-carboxylate-5,5,7,7-d₄ (330 mg, 1.3 mmol), tris(dibenzylideneacetone)dipalladium(0) (119 mg, 0.1 mmol), xantphos (113 mg, 0.2 mmol), and cesium carbonate (1.7 g, 5.2 mmol) in 1,4-Dioxane (6.5 mL). The reaction was degassed and heated ay 100° C. for 2 h. The mixture was cooled, filtered through a pad of Celite®, and washed with 3:1 CHCl₃/IPA. The solvents were removed, and the crude product was purified by normal-phase chromatography (0-5% MeOH/DCM) to afford the title compound (314 mg). ¹H NMR (400 MHz, CDCl₃) δ 8.38 (d, J=2.5 Hz, 1H), 8.32 (d, J=2.9 Hz, 1H), 8.13 (d, J=5.5 Hz, 1H), 7.33 (d, J=2.5 Hz, 1H), 6.94 (dd, J=7.0, 5.8, 1H), 6.24 (s, 1H), 3.00 (s, 2H), 1.50 (s, 9H); ES-MS [M+1]⁺: 349.3.

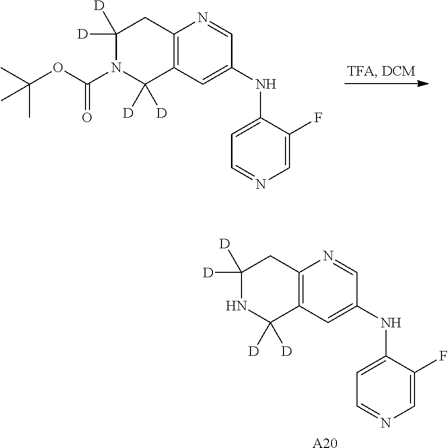 [SMILES: [2H]C1([2H])Cc2ncc(Nc3ccncc3F)cc2C([2H])([2H])N1C(=O)OC(C)(C)C ; [2H]C1([2H])Cc2ncc(Nc3ccncc3F)cc2C([2H])([2H])N1 ; "A20"]

N-(3-Fluoropyridin-4-yl)-5,6,7,8-tetrahydro-1,6-naphthyridin-5,5,7,7-d₄-3-amine (Intermediate A20). In a vial were combined tert-butyl 3-((3-fluoropyridin-4-yl)amino)-7,8-dihydro-1,6-naphthyridine-6(5H)-carboxylate-5,5,7,7-d₄ (314 mg, 0.9 mmol), trifluoroacetic acid (1.07 mL, 14.0 mmol), and DCM (4 mL). The reaction stirred at room temperature for 2 h. The reaction was concentrated and purified by SCX cartridge, eluting with 2N NH₃/MeOH solution. Solvents were removed to afford the title compound (178 mg). ¹H NMR (400 MHz, CDCl₃) δ 8.30 (d, J=2.5 Hz, 1H), 8.23 (s, 1H), 8.17 (d, J=5.7 Hz, 1H), 7.22 (d, J=2.6 Hz, 1H), 6.79 (d, J=5.6, 1H), 5.68 (s, 1H), 2.94 (s, 2H); ES-MS [M+1]⁺: 249.3.

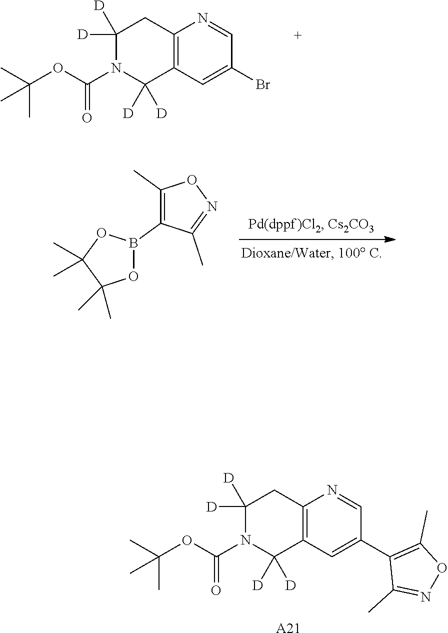

tert-Butyl 3-(3,5-dimethylisoxazol-4-yl)-7,8-dihydro-1,6-naphthyridine-6(5H)-carboxylate-5,5,7,7-d₄ (Intermediate A21). tert-Butyl 3-bromo-7,8-dihydro-1,6-naphthyridine-6(5H)-carboxylate-5,5,7,7-d₄ (200 mg, 0.63 mmol), 3,5-dimethylisoxazole-4-boronic acid pinacol ester (169 mg, 0.76 mmol), cesium carbonate (413 mg, 1.26 mmol), and Pd(dppf)Cl₂ (69 mg, 0.09 mmol) were combinedin 1,4-dioxane (2.5 mL) and water (0.5 mL) and degassed (3×). The reaction heated at 100° C. for 2.5 h. The mixture was filtered through a pad of Celite®, washed with EtOAc/DCM, and the filtrate was concentrated under reduced pressure. The oil was purified by normal-phase chromatography (0-4% MeOH/DCM) to afford the title compound (202 mg). ¹HNMR (400 MHz, CDCl₃) δ 8.34 (d, J=2.0 Hz, 1H), 7.31 (d, J=2.1 Hz, 1H), 3.04 (s, 2H), 2.41 (s, 3H), 2.27 (s, 3H), 1.51 (s, 9H); ES-MS [M+1]⁺: 334.1.

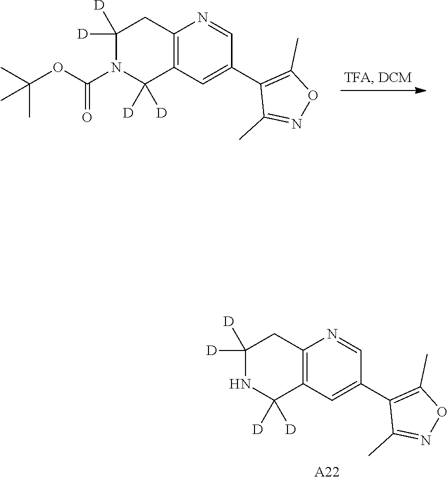 [SMILES: [2H]C1([2H])Cc2ncc(-c3c(C)noc3C)cc2C([2H])([2H])N1C(=O)OC(C)(C)C ; [2H]C1([2H])Cc2ncc(-c3c(C)noc3C)cc2C([2H])([2H])N1 ; "A22"]

3,5-Dimethyl-4-(5,6,7,8-tetrahydro-1,6-naphthyridin-3-yl-5,5,7,7-d)isoxazole (Intermediate A22). tert-Butyl 3-(3,5-dimethylisoxazol-4-yl)-7,8-dihydro-1,6-naphthyridine-6(5H)-carboxylate-5,5,7,7-d₄ (200.mg, 0.6 mmol) was combined with DCM (3 mL) and trifluoroacetic acid (0.69 mL, 9.0 mmol). After 2 h, the reaction was complete and was concentrated in vacuo. The crude oil was purified by SCX Cartridge, washing with MeOH and eluting compound with 7N NH₃/MeOH solution. Solvents were removed to afford the title compound (114 mg). ¹H NMR (400 MHz, CDCl₃) δ 8.31 (d, J=2.2 Hz, 1H), 7.20 (d, J=2.2 Hz, 1H), 2.98 (s, 2H), 2.39 (s, 3H), 2.25 (s, 3H); ES-MS [M+1]⁺: 234.3.

b. Exemplified Compounds of the Invention

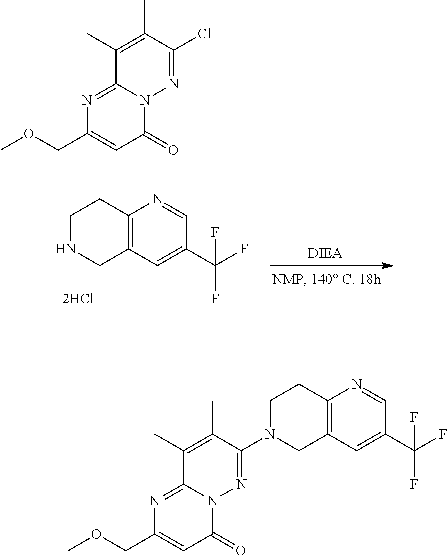 [SMILES: COCc1cc(=O)n2nc(Cl)c(C)c(C)c2n1 ; FC(F)(F)c1cnc2c(c1)CNCC2 ; COCc1cc(=O)n2nc(N3CCc4ncc(C(F)(F)F)cc4C3)c(C)c(C)c2n1]

2-(Methoxymethyl)-8,9-dimethyl-7-(3-(trifluoromethyl)-7,8-dihydro-1,6-naphthyridin-6(5H)-yl)-4H-pyrimido[1,2-b]pyridazin-4-one (Compound 1-109). To a solution of 7-chloro-2-(methoxymethyl)-8,9-dimethyl-pyrimido[1,2-b]pyridazin-4-one (100.0 mg, 0.39 mmol) in NMP (3.0 mL) were added DIEA (343 μL, 1.97 mmol) and 3-(trifluoromethyl)-5,6,7,8-tetrahydro-1,6-naphthyridine dihydrochloride (CAS #870483-68-4) (217 mg, 0.79 mmol). The mixture was heated to 140° C. for 18 h. Solvents were removed, and the crude product was dissolved in DMSO (5 mL) and purified by normal-phase chromatography (0-100% DCM/MeOH with 10% NH₄OH) to afford the title compound (130 mg). ¹H NMR (400 MHz, CDCl₃) δ 8.73 (d, J=1.8 Hz, 1H), 7.78 (d, J=1.9 Hz, 1H), 6.74 (d, J=1.0 Hz, 1H), 4.70 (s, 2H), 4.45 (d, J=1.0 Hz, 2H), 3.55 (t, J=5.9 Hz, 2H), 3.51 (s, 3H), 3.33 (t, J=5.9 Hz, 2H), 2.55 (d, J=1.0 Hz, 3H), 2.42 (d, J=1.1 Hz, 3H); ES-MS [M+H]⁺=420.4.

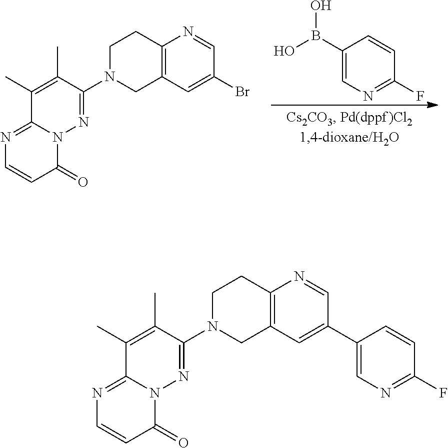 [SMILES: Cc1c(N2CCc3ncc(Br)cc3C2)nn2c(=O)ccnc2c1C ; OB(O)c1ccc(F)nc1 ; Cc1c(N2CCc3ncc(-c4ccc(F)nc4)cc3C2)nn2c(=O)ccnc2c1C]

7-(3-(6-Fluoropyridin-3-yl)-7,8-dihydro-1,6-naphthyridin-6(5H)-yl)-8,9-dimethyl-4H-pyrimido[1,2-b]pyridazin-4-one (Compound 1-87). 7-(3-Bromo-7,8-dihydro-1,6-naphthyridin-6(5H)-yl)-8,9-dimethyl-4H-pyrimido[1,2-b]pyridazin-4-one(200 mg, 0.52 mmol), (6-fluoropyridin-3-yl)boronic acid (219 mg, 1.55 mmol), cesium carbonate (337 mg, 1.03 mmol) and [1,1′-bis(diphenylphosphino)ferrocene)dichloropalladium(II)](19 mg, 0.02 mmol) were combined. Degassed 1,4-dioxane/water (5:1, v/v, 2.58 mL) was added. The reaction mixture was stirred at 100° C. After 16 h, the solvent was removed. The crude material was purified using reverse phase HPLC to provide the title compound (76 mg). ¹H NMR (400 MHz, DMSO) δ 9.08 (d, J=2.1 Hz, 1H), 8.76 (d, J=2.6 Hz, 1H), 8.66 (s, 1H), 8.50 (td, J=8.1, 2.7 Hz, 1H), 8.20 (d, J=6.3 Hz, 1H), 7.43 (dd, J=8.6, 2.8 Hz, 1H), 6.47 (d, J=6.3 Hz, 1H), 4.71 (s, 2H), 3.66-3.63 (m, 2H), 3.40-3.37 (m, 2H), 2.50 (s, 3H), 2.43 (s, 3H); ES-MS [M+1]⁺: 403.2.

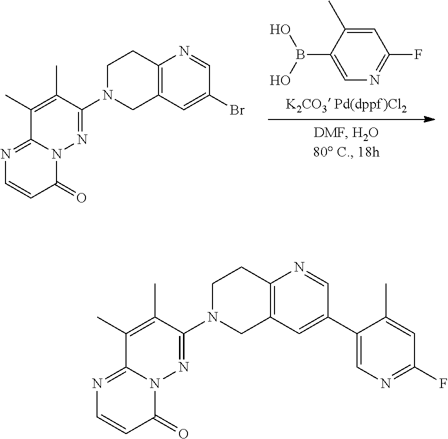 [SMILES: Cc1c(N2CCc3ncc(Br)cc3C2)nn2c(=O)ccnc2c1C ; Cc1cc(F)ncc1B(O)O ; Cc1cc(F)ncc1-c1cnc2c(c1)CN(c1nn3c(=O)ccnc3c(C)c1C)CC2]

7-(3-(6-Fluoro-4-methylpyridin-3-yl)-7,8-dihydro-1,6-naphthyridin-6(5H)-yl)-8,9-dimethyl-4H-pyrimido[1,2-b]pyridazin-4-one (Compound 1-149). Prepared in a similar manner as compound 1-91. ¹H NMR (400 MHz, CDCl₃) δ 8.43 (d, J=2.1 Hz, 1H), 8.19 (d, J=6.3 Hz, 1H), 8.06 (s, 1H), 7.55 (d, J=2.1 Hz, 1H), 6.90 (d, J=2.0 Hz, 1H), 6.58 (d, J=6.3 Hz, 1H), 4.74 (s, 2H), 3.59 (t, J=5.9 Hz, 2H), 3.35 (t, J=5.9 Hz, 2H), 2.58 (d, J=1.1 Hz, 3H), 2.47 (d, J=1.1 Hz, 3H), 2.35 (d, J=0.7 Hz, 3H); ES-MS [M+H]⁺=417.4.

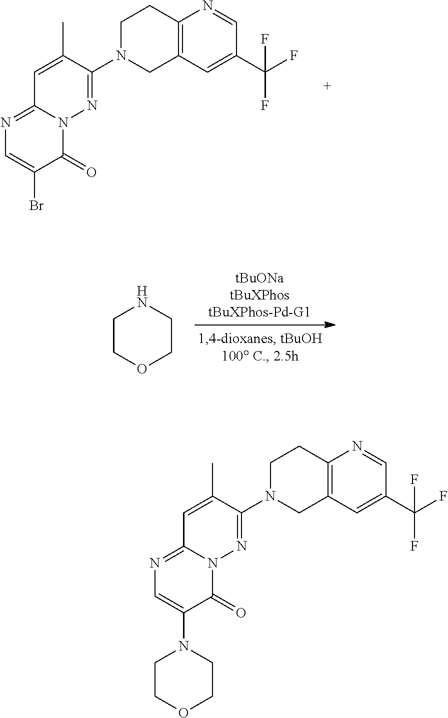 [SMILES: Cc1cc2ncc(Br)c(=O)n2nc1N1CCc2ncc(C(F)(F)F)cc2C1 ; C1COCCN1 ; Cc1cc2ncc(N3CCOCC3)c(=O)n2nc1N1CCc2ncc(C(F)(F)F)cc2C1]

8-Methyl-3-morpholino-7-(3-(trifluoromethyl)-7,8-dihydro-1,6-naphthyridin-6(5H)-yl)-4H-pyrimido[1,2-b]pyridazin-4-one (Compound 1-19). 3-Bromo-8-methyl-7-[3-(trifluoromethyl)-7,8-dihydro-5H-1,6-naphthyridin-6-yl]pyrimido[1,2-b]pyridazin-4-one (15 mg, 0.31 mmol), morpholine (3.0 mg, 0.031 mmol), sodium tert-butoxide (6 mg, 0.63 mmol), t-BuXPhos Palladacycle Gen 1 (3.0 mg, 0.005 mmol), t-BuXPhos (2.0 mg, 0.005 mmol), 1,4-dioxane (0.2 mL) and t-BuOH (0.3 mL) were combined in a vial and degassed (3×). The reaction was heated at 100° C. for 2.5 h. The reaction was filtered over Celite®, rinsed with DCM, and concentrated. The crude residue was purified by reverse-phase chromatography (5-45% MeCN/0.1% aqueous TFA). The desired fractions were neutralized with saturated aqueous bicarbonate solution and the fractions were then diluted with water and extracted with CHCl₃/IPA (3:1). The collected organic layers were dried (MgSO₄), filtered, and concentrated to afford the title compound (4 mg). ES-MS [M+H]⁺=447.4.

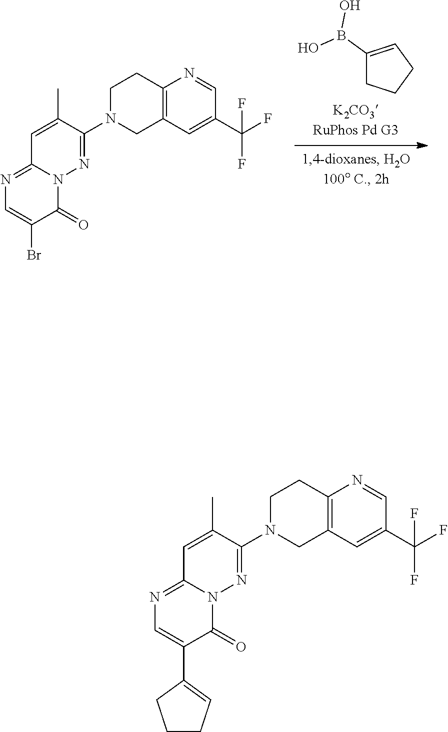 [SMILES: Cc1cc2ncc(Br)c(=O)n2nc1N1CCc2ncc(C(F)(F)F)cc2C1 ; OB(O)C1=CCCC1 ; Cc1cc2ncc(C3=CCCC3)c(=O)n2nc1N1CCc2ncc(C(F)(F)F)cc2C1]

3-(Cyclopent-1-en-1-yl)-8-methyl-7-(3-(trifluoromethyl)-7,8-dihydro-1,6-naphthyridin-6(5H)-yl)-4H-pyrimido[1,2-b]pyridazin-4-one (Compound 1-20). 3-Bromo-8-methyl-7-[3-(trifluoromethyl)-7,8-dihy dro-5H-1,6-naphthyridin-6-yl]pyrimido[1,2-b]pyridazin-4-one (20 mg, 0.045 mmol), cyclopenten-1-ylboronic acid (CAS #850036-28-1) (5.0 mg, 0.045 mmol), potassium carbonate (29 mg, 0.20 mmol), RuPhos Palladacycle Gen 3 (5.7 mg, 0.007 mmol), 1,4-dioxane (0.4 mL) and water (0.4 mL) were combined in a vial and degassed (3×). The reaction was heated at 100° C. for 2 h. The reaction was filtered over Celite®, rinsed with DCM, and concentrated. The crude oil was purified by reverse-phase HPLC (5-45% MeCN/0.1% aqueous TFA). The desired fractions were neutralized with saturated aqueous bicarbonate solution and the fractions were then diluted with water, extracted with CHCl₃/IPA (3:1). The collected organic layers were dried (MgSO₄), filtered and concentrated to afford the title compound (4.2 mg). ES-MS [M+H]⁺=428.3.

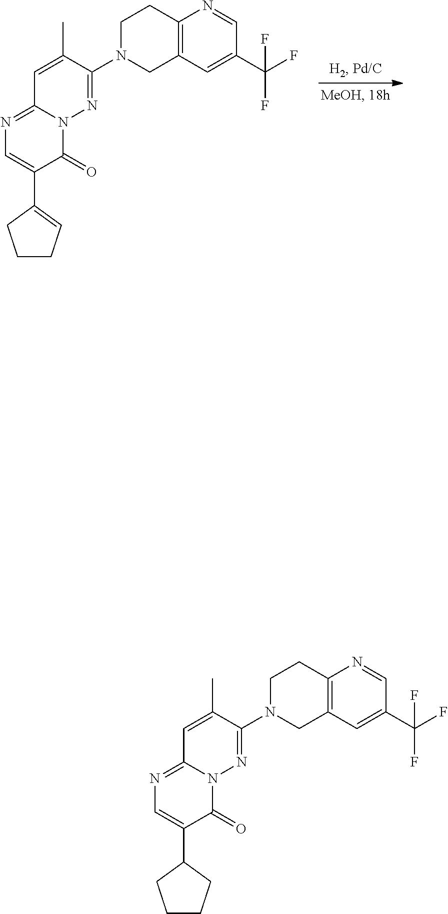 [SMILES: Cc1cc2ncc(C3=CCCC3)c(=O)n2nc1N1CCc2ncc(C(F)(F)F)cc2C1 ; Cc1cc2ncc(C3CCCC3)c(=O)n2nc1N1CCc2ncc(C(F)(F)F)cc2C1]

3-Cyclopentyl-8-methyl-7-(3-(trifluoromethyl)-7,8-dihydro-1,6-naphthyridin-6(5H)-yl)-4H-pyrimido[1,2-b]pyridazin-4-one (Compound 1-23). 3-(Cyclopenten-1-yl)-8-methyl-7-[3-(trifluoromethyl)-7,8-dihy dro-5H-1,6-naphthyridin-6-yl]pyrimido[1,2-b]pyridazin-4-one (10 mg, 0.02 mmol) was suspended in methanol (3.0 mL), palladium on activated carbon (25 mg) was added, the flask was evacuated and purged with H₂ (1 atm). After 18 h at rt, the reaction was filtered, diluted with DMSO, and purified by reverse-phase chromatography (MeCN/0.1% aqueous TFA) to afford the title compound (6.6 mg). ¹H NMR (400 MHz, CDCl₃) δ 8.73 (dd, J=2.3, 1.1 Hz, 1H), 8.08 (d, J=0.8 Hz, 1H), 7.75 (dd, J=2.0, 1.0 Hz, 1H), 7.53 (q, J=1.2 Hz, 1H), 4.75 (s, 2H), 3.63 (t, J=5.9 Hz, 2H), 3.32 (t, J=6.0 Hz, 2H), 3.28-3.15 (m, 1H), 2.49 (d, J=1.2 Hz, 3H), 2.13-2.02 (m, 2H), 1.90-1.80 (m, 2H), 1.79-1.67 (m, 4H). ES-MS [M+H]⁺=430.4.

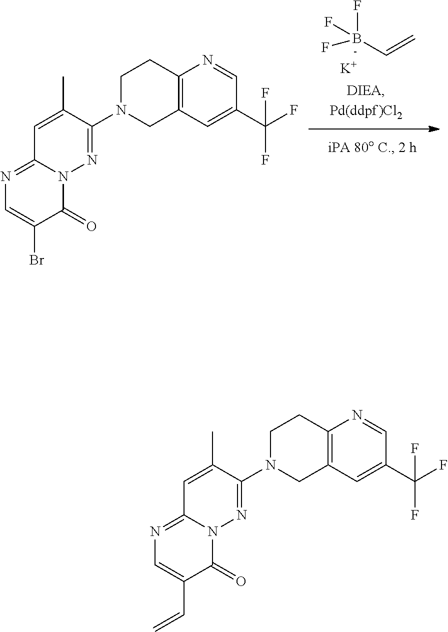

8-Methyl-7-(3-(trifluoromethyl)-7,8-dihydro-1,6-naphthyridin-6(5H)-yl)-3-vinyl-4H-pyrimido[1,2-b]pyridazin-4-one (Compound 1-22). 3-Bromo-8-methyl-7-[3-(trifluoromethyl)-7,8-dihydro-5H-1,6-naphthyridin-6-yl]pyrimido[1,2-b]pyridazin-4-one (20 mg, 0.045 mmol), potassium vinyltrifluoroborate (9 mg, 0.068 mmol), DIEA (0.36 μL, 0.20 mmol), Pd(dppf)Cl₂ (5 mg, 0.007 mmol), and iPA (0.5 mL) were combined in a vial and degassed (3×). The reaction was heated at 80° C. for 2 h. The reaction was filtered over Celite®, rinsed with DCM, and concentrated. The crude residue was purified by reverse-phase chromatography (5-45% MeCN/0.1% aqueous TFA). The desired fractions were neutralized with saturated aqueous bicarbonate solution, and the fractions were then diluted with water, extracted with CHCl₃/IPA (3:1). The collected organic layers were dried (MgSO₄), filtered and concentrated to afford the title compound (4.7 mg). ES-MS [M+H]⁺=388.4.

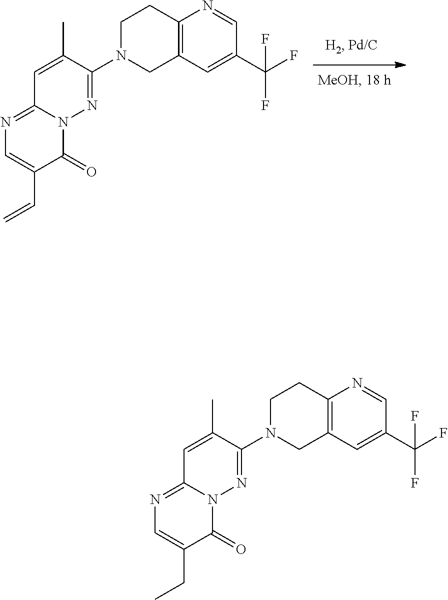 [SMILES: C=Cc1cnc2cc(C)c(N3CCc4ncc(C(F)(F)F)cc4C3)nn2c1=O ; CCc1cnc2cc(C)c(N3CCc4ncc(C(F)(F)F)cc4C3)nn2c1=O]

3-Ethyl-8-methyl-7-(3-(trifluoromethyl)-7,8-dihydro-1,6-naphthyridin-6(5H)-yl)-4H-pyrimido[1,2-b]pyridazin-4-one (Compound 1-24). 8-Methyl-7-[3-(trifluoromethyl)-7,8-dihydro-5H-1,6-naphthyridin-6-yl]-3-vinyl-pyrimido[1,2-b]pyridazin-4-one (9 mg, 0.02 mmol) was suspended in methanol (3 mL), palladium on activated carbon (25 mg) was added, the flask was evacuated and purged with H₂ (1 atm). After 18 h at rt, the reaction was filtered, diluted with DMSO and purified by reverse-phase chromatography (MeCN/0.1% aqueous TFA) to afford the title compound (5.2 mg). ES-MS [M+H]⁺=390.4.

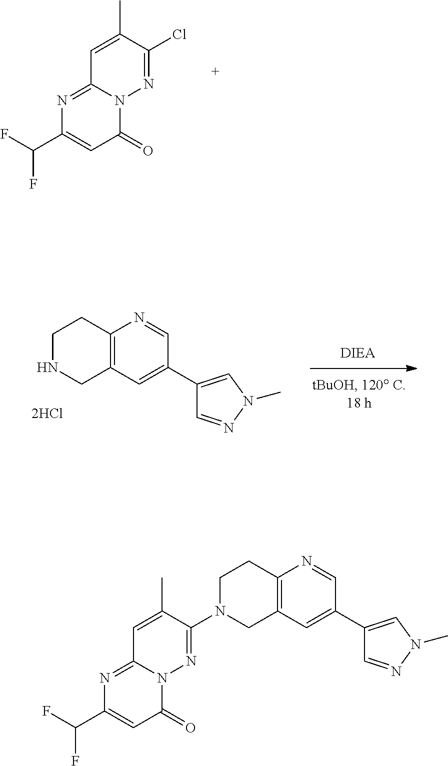 [SMILES: Cc1cc2nc(C(F)F)cc(=O)n2nc1Cl ; Cn1cc(-c2cnc3c(c2)CNCC3)cn1 ; Cc1cc2nc(C(F)F)cc(=O)n2nc1N1CCc2ncc(-c3cnn(C)c3)cc2C1]

2-(Difluoromethyl)-8-methyl-7-(3-(1-methyl-1H-pyrazol-4-yl)-7,8-dihydro-1,6-naphthyridin-6(5H)-yl)-4H-pyrimido[1,2-b]pyridazin-4-one (Compound 1-35). To a solution of 3-(1-methyl-1H-pyrazol-4-yl)-5,6,7,8-tetrahydro-1,6-naphthyridine dihydrochloride (18 mg, 0.061 mmol) in tert-butanol (1 mL) was added DIEA (35 μL, 0.20 mmol) and 7-chloro-2-(difluoromethyl)-8-methyl-pyrimido[1,2-b]pyridazin-4-one (10 mg, 0.041 mmol). The mixture was heated to 120° C. for 18 h. Solvents were removed, and the crude product was dissolved in DMSO (2 mL) and purified by reverse-phase chromatography (MeCN/0.05% aqueous NH₄OH) to afford the title compound (11.2 mg). ¹H NMR (400 MHz, CDCl₃) δ 8.61 (d, J=2.2 Hz, 1H), 7.78 (d, J=0.9 Hz, 1H), 7.66 (d, J=0.8 Hz, 1H), 7.64 (t, J=1.3 Hz, 1H), 7.62 (d, J=2.2 Hz, 1H), 6.85 (s, 1H), 6.46 (t, J=55.1 Hz, 1H), 4.74 (s, 2H), 3.98 (s, 3H), 3.66 (t, J=5.9 Hz, 2H), 3.26 (t, J=5.9 Hz, 2H), 2.56 (d, J=1.2 Hz, 3H); ES-MS [M+H]⁺=424.2.

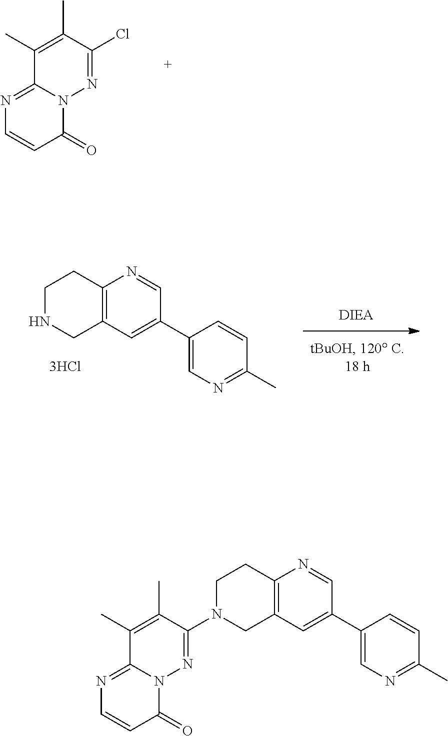 [SMILES: Cc1c(Cl)nn2c(=O)ccnc2c1C ; Cc1ccc(-c2cnc3c(c2)CNCC3)cn1 ; Cc1ccc(-c2cnc3c(c2)CN(c2nn4c(=O)ccnc4c(C)c2C)CC3)cn1]

8,9-Dimethyl-7-(3-(6-methylpyridin-3-yl)-7,8-dihydro-1,6-naphthyridin-6(5H)-yl)-4H-pyrimido[1,2-b]pyridazin-4-one (Compound 1-61). To a solution of 3-(6-methyl-3-pyridyl)-5,6,7,8-tetrahydro-1,6-naphthyridine trihydrochloride (184 mg, 0.55 mmol) in tert-butanol (5 mL) was added DIEA (320 μL, 1.84 mmol) and 7-chloro-8,9-dimethyl-pyrimido[1,2-b]pyridazin-4-one (77 mg, 0.37 mmol). The mixture was heated to 120° C. for 18 h. Solvents were removed, and the crude product was dissolved in DMSO (5 mL) and purified by reverse-phase chromatography (MeCN/0.05% aqueous NH₄OH) to afford the title compound (44 mg). ¹H NMR (400 MHz, DMSO) δ 8.83 (dd, J=2.5, 0.8 Hz, 1H), 8.77 (d, J=2.3 Hz, 1H), 8.19 (d, J=6.2 Hz, 1H), 8.09-8.02 (m, 2H), 7.41-7.34 (m, 1H), 6.46 (d, J=6.2 Hz, 1H), 4.60 (s, 2H), 3.58 (t, J=5.9 Hz, 2H), 3.20 (t, J=5.8 Hz, 2H), 2.52 (s, 3H), 2.49 (s, 3H), 2.43 (d, J=1.1 Hz, 3H); ES-MS [M+H]⁺=399.2.

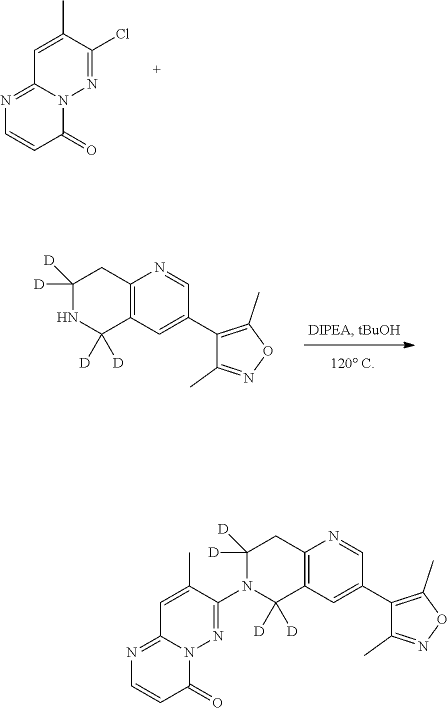 [SMILES: Cc1cc2nccc(=O)n2nc1Cl ; [2H]C1([2H])Cc2ncc(-c3c(C)noc3C)cc2C([2H])([2H])N1 ; [2H]C1([2H])Cc2ncc(-c3c(C)noc3C)cc2C([2H])([2H])N1c1nn2c(=O)ccnc2cc1C]

7-(3-(3,5-Dimethylisoxazol-4-yl)-7,8-dihydro-1,6-naphthyridin-6(5H)-yl-5,5,7,7-d₄)-8-methyl-4H-pyrimido[1,2-b]pyridazin-4-one (Compound 1-450). In a vial were combined 7-chloro-8-methylpyrimido[1,2-b]pyridazin-4-one (12 mg, 0.06 mmol), N,N-diisopropylethylamine (53 μL, 0.31 mmol), and Intermediate A22 (14 mg, 0.06 mmol) in tert-butanol (0.8 mL). The reaction was heated to 120° C. After 18 h, the solvents were removed, and the crude oil was purified by reverse-phase chromatography to afford the title compound (7 mg). ¹H NMR (400 MHz, CDCl₃) δ 8.40 (d, J=2.2 Hz, 1H), 8.15 (d, J=6.4 Hz, 1H), 7.59 (d, J=1.2 Hz, 1H), 7.47 (d, J=2.2 Hz, 1H), 6.58 (d, J=6.4 Hz, 1H), 3.29 (s, 2H), 2.55 (d, J=1.1 Hz, 3H), 2.44 (s, 3H), 2.30 (s, 3H); ES-MS [M+1]⁺: 393.5

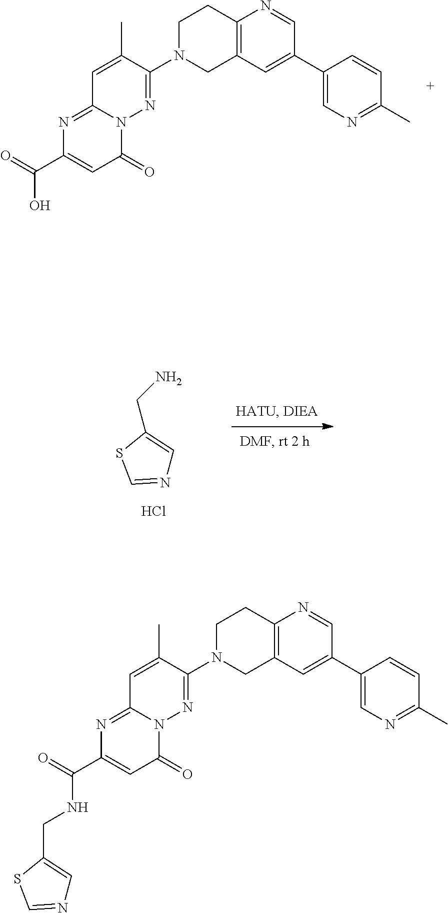 [SMILES: Cc1ccc(-c2cnc3c(c2)CN(c2nn4c(=O)cc(C(=O)O)nc4cc2C)CC3)cn1 ; NCc1cncs1 ; Cl ; Cc1ccc(-c2cnc3c(c2)CN(c2nn4c(=O)cc(C(=O)NCc5cncs5)nc4cc2C)CC3)cn1]

8-Methyl-7-(3-(6-methylpyridin-3-yl)-7,8-dihydro-1,6-naphthyridin-6(5H)-yl)-4-oxo-N-(thiazol-5-ylmethyl)-4H-pyrimido[1,2-b]pyridazine-2-carboxamide (Compound 1-77). 8-Methyl-7-(3-(6-methylpyridin-3-yl)-7,8-dihy dro-1,6-naphthyridin-6(5H)-yl)-4-oxo-4H-pyrimido[1,2-b]pyridazine-2-carboxylic acid (6.3 mg, 0.01 mmol) and HATU (14.9 mg, 0.04 mmol) were dissolved in DMF (0.5 mL). After 15 min at rt, DIEA (10 μL, 0.06 mmol) and 5-(aminomethyl)thiazole hydrochloride (131052-46-5) (2 mg, 0.13 mmol) were added to the mixture. After 2 h at rt, the crude mixture was filtered and purified by reverse-phase chromatography (0-45% MeCN/0.05% aqueous NH₄OH) to afford the title compound (1.9 mg). ES-MS [M+H]⁺=525.1.

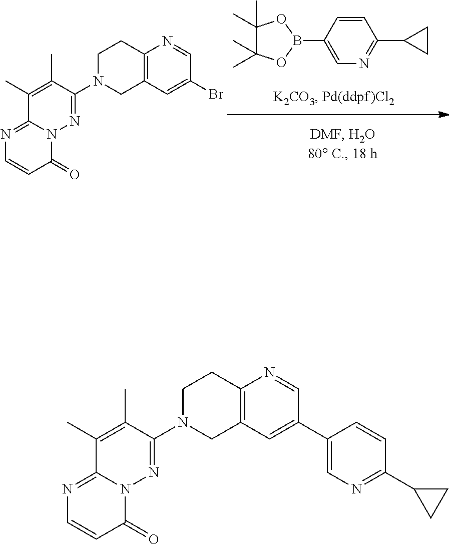

7-(3-(6-Cyclopropylpyridin-3-yl)-7,8-dihydro-1,6-naphthyridin-6(5H)-yl)-8,9-dimethyl-4H-pyrimido[1,2-b]pyridazin-4-one (Compound 1-91). 7-(3-Bromo-7,8-dihydro-5H-1,6-naphthyridin-6-yl)-8,9-dimethyl-pyrimido[1,2-b]pyridazin-4-one (15 mg, 0.039 mmol), 2-cyclopropyl-5-pyridine boronic acid pinacol ester (CAS #893567-09-4) (19 mg, 0.078 mmol), potassium carbonate (22 mg, 0.16 mmol), Pd(dppf)Cl₂ (6 mg, 0.008 mmol), DMF (0.8 mL) and water (0.2 mL) were combined in a vial and degassed (3×). The reaction was heated at 80° C. for 18 h. The reaction was filtered over Celite®, rinsed with DCM, and concentrated. The crude resiude was purified by reverse-phase chromatography (5-45% MeCN/0.10% aqueous TFA). The desired fractions were neutralized with saturated aqueous bicarbonate solution and the fractions were then diluted with water, extracted with CHCl₃/IPA (3.1). The collected organic layers were dried (MgSO₄), filtered and concentrated to afford the title compound (8.2 mg). ¹H NMR (400 MHz, CDCl₃) δ 8.67 (d, J=2.3 Hz, 2H), 8.18 (d, J=6.3 Hz, 1H), 7.78-7.71 (m, 2H), 7.26 (dd, J=8.1, 0.8 Hz, 1H), 6.59 (d, J=6.3 Hz, 1H), 4.73 (s, 2H), 3.57 (t, J=5.9 Hz, 2H), 3.32 (t, J=5.9 Hz, 2H), 2.58 (d, J=1.0 Hz, 3H), 2.46 (s, 2H), 2.18-2.07 (m, 2H), 1.14-1.01 (m, 4H); ES-MS [M+H]⁺=425.4.

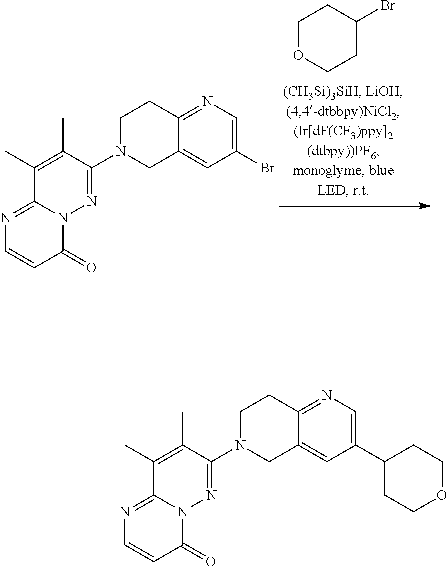 [SMILES: BrC1CCOCC1 ; Cc1c(N2CCc3ncc(Br)cc3C2)nn2c(=O)ccnc2c1C ; Cc1c(N2CCc3ncc(C4CCOCC4)cc3C2)nn2c(=O)ccnc2c1C]

8,9-Dimethyl-7-(3-(tetrahydro-2H-pyran-4-yl)-7,8-dihydro-1,6-naphthyridin-6(5H)-yl)-4H-pyrimido[1,2-b]pyridazin-4-one (Compound 1-429). 7-(3-Bromo-7,8-dihydro-1,6-naphthyridin-6(5H)-yl)-8,9-dimethyl-4H-pyrimido[1,2-b]pyridazin-4-one (20 mg, 0.52 mmol), lithium hydroxide (3.7 mg, 0.16 mmol) and (Ir[dF(CF₃)ppy]₂(dtbpy))PF₆ (2.9 mg, 0.003 mmol; CAS #870987-63-6) were combined in a vial, followed by the addition 4-bromotetrahydro-2H-pyran (0.017 mL, 0.16 mmol) and tris(trimethylsilyl)silane (0.024 mL, 0.078 mmol). Monoglyme (1 mL) was added, and the resulting reaction mixture was briefly stirred under vacuum, followed by degassing via bubbling N₂ gas into the reaction mixture. (4,4′-dtbbpy)NiCl₂ (1.0 mg, 0.003 mmol) was then added, followed by additional stirring under vacuum and N₂ purging. The resulting reaction mixture was sonicated and then stirred under an inert atmosphere with blue LED irradiation (EvoluChem™ PhotoRedOx Box equipped with Kessil® LED lamp (HepatoChem)) overnight at ambient temperature, after which time the reaction mixture was diluted with sat. NaHCO₃ solution and DCM. The aqueous layer was extracted with DCM, and the combined organic extracts were filtered through a phase separator, and concentrated. The crude residue was purified by RP-HPLC (10-50% MeCN/0.05% aqueous NH₄OH). The fractions containing product were concentrated to afford 2.5 mg of the title compound. ¹H NMR (400 MHz, CDCl₃) δ 8.36 (d, J=2.2 Hz, 1H), 8.18 (d, J=6.3 Hz, 1H), 7.41 (d, J=2.2 Hz, 1H), 6.58 (d, J=6.3 Hz, 1H), 4.65 (s, 2H), 4.13-4.08 (m, 2H), 3.58-3.48 (m, 4H), 3.24 (t, J=5.9 Hz, 2H), 2.84-2.76 (m, 1H), 2.57 (d, J=1.0 Hz, 3H), 2.44 (d, J=1.0 Hz, 3H), 1.87-1.76 (m, 4H); ES-MS [M+1]⁺: 392.1.

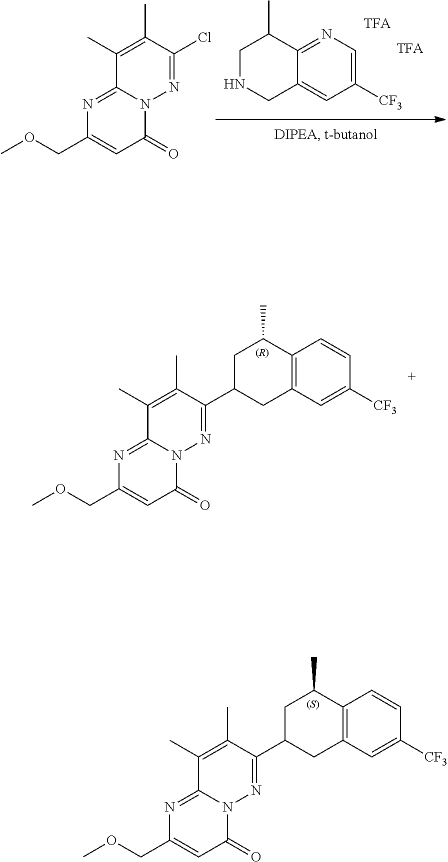

(S)-2-(Methoxymethyl)-8,9-dimethyl-7-(8-methyl-3-(trifluoromethyl)-7,8-dihydro-1,6-naphthyridin-6(5H)-yl)-4H-pyrimido[1,2-b]pyridazin-4-one and (R)-2-(methoxymethyl)-8,9-dimethyl-7-(8-methyl-3-(trifluoromethyl)-7,8-dihydro-1,6-naphthyridin-6(5H)-yl)-4H-pyrimido[1,2-b]pyridazin-4-one. 7-Chloro-2-(methoxymethyl)-8,9-dimethyl-pyrimido[1,2-b]pyridazin-4-one (40 mg), 8-methyl-3-(trifluoromethyl)-5,6,7,8-tetrahydro-1,6-naphthyridine;2,2,2-trifluoroacetic acid (75 mg), and NN-diisopropylethylamine (137 μL) were combined in a vial followed by the addition of tert-butanol (0.5 mL). The reaction was heated at 120° C. for 18 h. The reaction was purified by reverse-phase HPLC to afford the racemic mixture. The sample was purified by SFC to afford the enantiomerically pure title compounds.

### Analytical Separation Example:

Chiral SFC separation was performed on a Thar (Waters) Investigator. Column: Chiral Technologies CHIRALPAK IA, 4.6×250 mm, 5 μm. Gradient conditions: 25% ethanol in CO₂ over 10 min. Flow rate: 3.5 mL/min. Column temperature: 40° C. System backpressure: 100 bar. Compound 1-376: Compound 1-377(1:1)

### Preparative Separation Example:

Chiral SFC separation was performed on a PIC Solution SFC-PICLab PREP 100. Column: Chiral Technologies CHIRALPAK IA, 20×250 mm, 5 μm. Conditions: 25% ethanol in CO₂. Flow rate: 80 mL/min. Column temperature: 40° C. System backpressure: 100 bar.

Compound 1-376 (first eluted peak):

Rt=8.47 mi (preparative method); ES-MS [M+H]⁺=434.2.; 99.9% ee. ¹H NMR (400 MHz, CDCl₃) δ 8.77 (s, 1H), 7.77 (s, 1H), 6.72 (s, 1H), 4.67-4.65 (m, 2H), 4.45 (s, 2H), 3.68 (dd, J=13.1, 4.9 Hz, 1H), 3.50 (s, 3H), 3.48-3.44 (m, 1H), 3.22 (dd, J=13.0, 7.7 Hz, 1H), 2.54 (s, 3H), 2.43 (s, 3H), 1.51 (d, J=7.1 Hz, 3H); ES-MS [M+1]M⁺: 434.2

Compound 1-377 (second eluted peak):

Rt=6.02 min (preparative method; ES-MS [M+H]⁺=434.2; 98.8% ee.

The compounds shown in Table 1 may be prepared using the methods shown in the preceding Schemes and Examples with the appropriate starting materials.

TABLE 1

| Cpd. | | | ES-MS |
|---|---|---|---|
| No. | Name | Structure | [M + 1]⁺ |
| 1-1 | 8-methyl-7-(3-(trifluoromethyl)- 7,8-dihydro-1,6-naphthyridin- 6(5H)-yl)-4H-pyrimido[1,2- b]pyridazin-4-one | 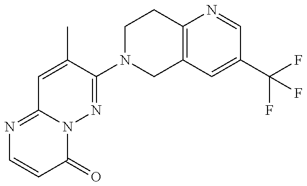 [SMILES: Cc1cc2nccc(=O)n2nc1N1CCc2ncc(C(F)(F)F)cc2C1] | 362.2 |
| 1-2 | 7-(3-(1,3-dimethyl-1H-pyrazol-4- yl)-7,8-dihydro-1,6-naphthyridin- 6(5H)-yl)-8-methyl-4H- pyrimido[1,2-b]pyridazin-4-one | 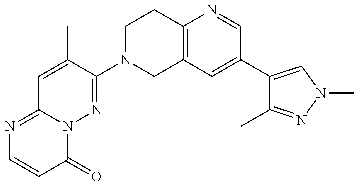 [SMILES: Cc1cc2nccc(=O)n2nc1N1CCc2ncc(-c3cn(C)nc3C)cc2C1] | 388.2 |
| 1-3 | 8-methyl-7-(3-(6-methylpyridin-3- yl)-7,8-dihydro-1,6-naphthyridin- 6(5H)-yl)-4H-pyrimido[1,2- b]pyridazin-4-one | 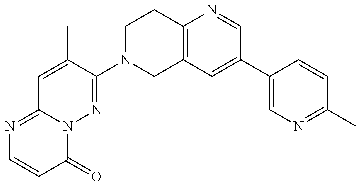 [SMILES: Cc1ccc(-c2cnc3c(c2)CN(c2nn4c(=O)ccnc4cc2C)CC3)cn1] | 385.2 |
| 1-4 | 3-chloro-8-methyl-7-(3- (trifluoromethyl)-7,8-dihydro-1,6- napthyridin-6(5H)-yl)-4H- pyrimido[1,2-b]pyridazin-4-one | 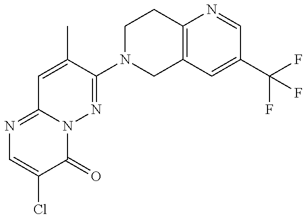 [SMILES: Cc1cc2ncc(Cl)c(=O)n2nc1N1CCc2ncc(C(F)(F)F)cc2C1] | 396 |
| 1-5 | 8-methyl-7-(3-(2-methylthiazol-5- yl)-7,8-dihydro-1,6-naphthyridin- 6(5H)-yl)-4H-pyrimido[1,2- b]pyridazin-4-one | 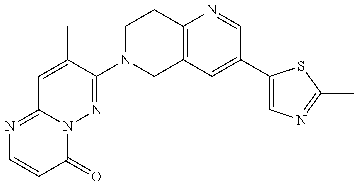 [SMILES: Cc1ncc(-c2cnc3c(c2)CN(c2nn4c(=O)ccnc4cc2C)CC3)s1] | 391.4 |
| 1-6 | 8-methyl-7-(3-(1-methyl-1H- pyrazol-4-yl)-7,8-dihydro-1,6- naphthyridin-6(5H)-yl)-4H- pyrimido[1,2-b]pyridazin-4-one | 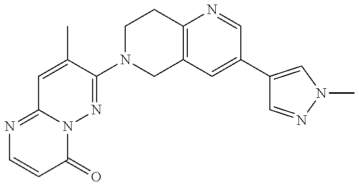 [SMILES: Cc1cc2nccc(=O)n2nc1N1CCc2ncc(-c3cnn(C)c3)cc2C1] | 374.4 |
| 1-7 | 8-methyl-7-(3-(1-methyl-1H- pyrazol-5-yl)-7,8-dihydro-1,6- naphthyridin-6(5H)-yl)-4H- pyrimido[1,2-b]pyridazin-4-one | 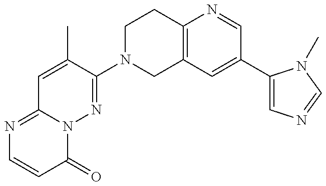 [SMILES: Cc1cc2nccc(=O)n2nc1N1CCc2ncc(-c3cncn3C)cc2C1] | 374.4 |
| 1-8 | 7-(3-(1-cyclopropyl-1H-pyrazol-4- yl)-7,8-dihydro-1,6-naphthyridin- 6(5H)-yl)-8-methyl-4H- pyrimido[1,2-b]pyridazin-4-one | 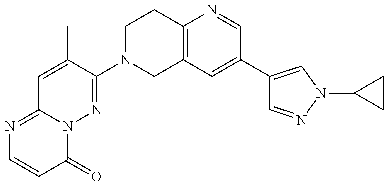 [SMILES: Cc1cc2nccc(=O)n2nc1N1CCc2ncc(-c3cnn(C4CC4)c3)cc2C1] | 400.4 |
| 1-9 | 3-chloro-7-(3-cyclopropyl-7,8- dihydro-1,6-naphthyridin-6(5H)- yl)-8-methyl-4H-pyrimido[1,2- b]pyridazin-4-one | 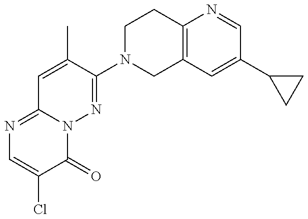 [SMILES: Cc1cc2ncc(Cl)c(=O)n2nc1N1CCc2ncc(C3CC3)cc2C1] | 368.2 |
| 1-10 | 3-chloro-8-methyl-7-(3-(1-methyl- 1H-pyrazol-5-yl)-7,8-dihydro-1,6- naphthyridin-6(5H)-yl)-4H- pyrimido[1,2-b]pyridazin-4-one | 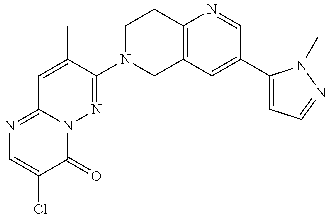 [SMILES: Cc1cc2ncc(Cl)c(=O)n2nc1N1CCc2ncc(-c3ccnn3C)cc2C1] | 408 |
| 1-11 | 3-chloro-7-(3-(1,3-dimethyl-1H- pyrazol-4-yl)-7,8-dihydro-1,6- naphthyridin-6(5H)-yl)-8-methyl- 4H-pyrimido[1,2-b]pyridazin-4-one | 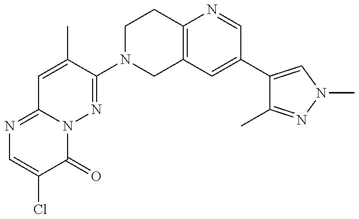 [SMILES: Cc1cc2ncc(Cl)c(=O)n2nc1N1CCc2ncc(-c3cn(C)nc3C)cc2C1] | 422.2 |
| 1-12 | 3-chloro-7-(3-(1-cyclopropyl-1H- pyrazol-4-yl)-7,8-dihydro-1,6- naphthyridin-6(5H)-yl)-8-methyl- 4H-pyrimido[1,2-b]pyridazin-4-one | 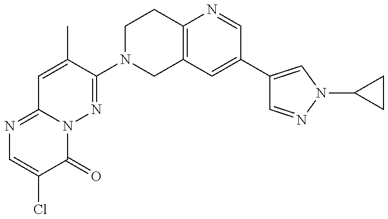 [SMILES: Cc1cc2ncc(Cl)c(=O)n2nc1N1CCc2ncc(-c3cnn(C4CC4)c3)cc2C1] | 434 |
| 1-13 | 3-chloro-8-methyl-7-(3-(2- methylthiazol-5-yl)-7,8-dihydro- 1,6-naphthyridin-6(5H)-yl)-4H- pyrimido[1,2-b]pyridazin-4-one | 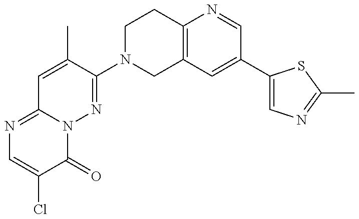 [SMILES: Cc1ncc(-c2cnc3c(c2)CN(c2nn4c(=O)c(Cl)cnc4cc2C)CC3)s1] | 425 |
| 1-14 | 3-chloro-8-methyl-7-(3-(6- methylpyridin-3-yl)-7,8-dihydro- 1,6-naphthyridin-6(5H)-yl)-4H- pyrimido[1,2-b]pyridazin-4-one | 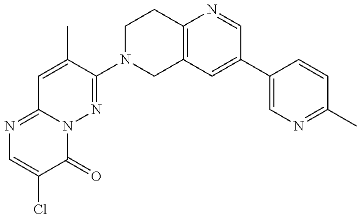 [SMILES: Cc1ccc(-c2cnc3c(c2)CN(c2nn4c(=O)c(Cl)cnc4cc2C)CC3)cn1] | 419.3 |
| 1-15 | 3-fluoro-2,8-dimethyl-7-(3- (trifluoromethyl)-7,8-dihydro-1,6- naphthyridin-6(5H)-yl)-4H- pyrimido[1,2-b]pyridazin-4-one | 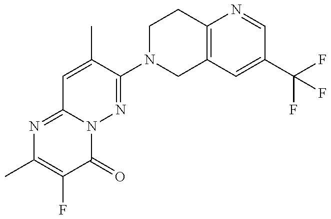 [SMILES: Cc1cc2nc(C)c(F)c(=O)n2nc1N1CCc2ncc(C(F)(F)F)cc2C1] | 394.2 |
| 1-16 | 2,8-dimethyl-7-(3- (trifluoromethyl)-7,8-dihydro-1,6- naphthyridin-6(5H)-yl)-4H- pyrimido[1,2-b]pyridazin-4-one | 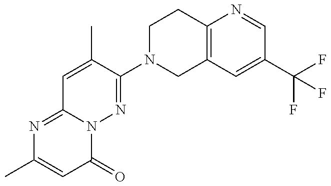 [SMILES: Cc1cc(=O)n2nc(N3CCc4ncc(C(F)(F)F)cc4C3)c(C)cc2n1] | 376.2 |
| 1-17 | 8-methyl-2-(trifluoromethyl)-7-(3- (trifluoromethyl)-7,8-dihydro-1,6- naphthyridin-6(5H)-yl)-4H- pyrimido[1,2-b]pyridazin-4-one | 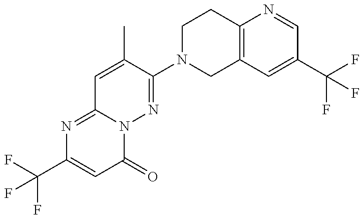 [SMILES: Cc1cc2nc(C(F)(F)F)cc(=O)n2nc1N1CCc2ncc(C(F)(F)F)cc2C1] | 430 |
| 1-18 | 3-fluoro-2,8,9-trimethyl-7-(3- (trifluoromethyl)-7,8-dihydro-1,6- naphthyridin-6(5H)-yl)-4H- pyrimido[1,2-b]pyridazin-4-one | 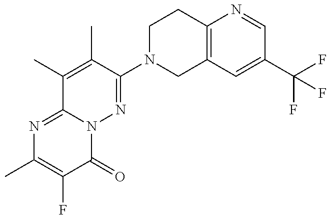 [SMILES: Cc1nc2c(C)c(C)c(N3CCc4ncc(C(F)(F)F)cc4C3)nn2c(=O)c1F] | 408.2 |
| 1-19 | 8-methyl-3-morpholino-7-(3- (trifluoromethyl)-7,8-dihydro-1,6- naphthyridin-6(5H)-yl)-4H- pyrimido[1,2-b]pyridazin-4-one | 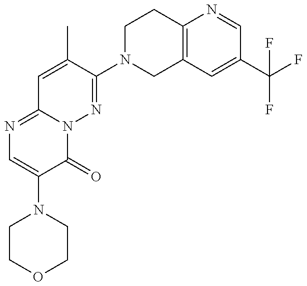 [SMILES: Cc1cc2ncc(N3CCOCC3)c(=O)n2nc1N1CCc2ncc(C(F)(F)F)cc2C1] | 447.4 |
| 1-20 | 3-(cyclopent-1-en-1-yl)-8-methyl-7- (3-(trifluoromethyl)-7,8-dihydro- 1,6-naphthyridin-6(5H)-yl)-4H- pyrimido[1,2-b]pyridazin-4-one | 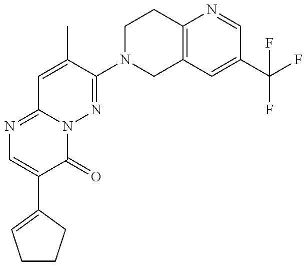 [SMILES: Cc1cc2ncc(C3=CCCC3)c(=O)n2nc1N1CCc2ncc(C(F)(F)F)cc2C1] | 428.3 |
| 1-21 | 8-methyl-3-phenyl-7-(3- (trifluoromethyl)-7,8-dihydro-1,6- naphthyridin-6(5H)-yl)-4H- pyrimido[1,2-b]pyridazin-4-one | 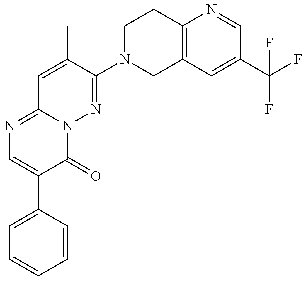 [SMILES: Cc1cc2ncc(-c3ccccc3)c(=O)n2nc1N1CCc2ncc(C(F)(F)F)cc2C1] | 438.4 |
| 1-22 | 8-methyl-7-(3-(trifluoromethyl)- 7,8-dihydro-1,6-naphthyridin- 6(5H)-yl)-3-vinyl-4H-pyrimido[1,2- b]pyridazin-4-one | 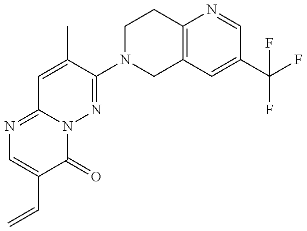 [SMILES: C=Cc1cnc2cc(C)c(N3CCc4ncc(C(F)(F)F)cc4C3)nn2c1=O] | 388.4 |
| 1-23 | 3-cyclopentyl-8-methyl-7-(3- (trifluoromethyl)-7,8-dihydro-1,6- naphthyridin-6(5H)-yl)-4H- pyrimido[1,2-b]pyridazin-4-one | 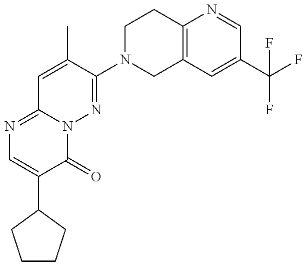 [SMILES: Cc1cc2ncc(C3CCCC3)c(=O)n2nc1N1CCc2ncc(C(F)(F)F)cc2C1] | 430.4 |
| 1-24 | 3-ethyl-8-methyl-7-(3- (trifluoromethyl)-7,8-dihydro-1,6- naphthyridin-6(5H)-yl)-4H- pyrimido[1,2-b]pyridazin-4-one | 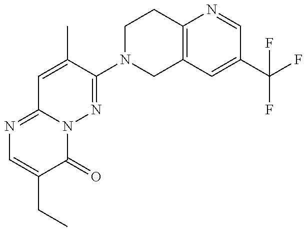 [SMILES: CCc1cnc2cc(C)c(N3CCc4ncc(C(F)(F)F)cc4C3)nn2c1=O] | 390.4 |
| 1-25 | 2,3,8-trimethyl-7-(3- (trifluoromethyl)-7,8-dihydro-1,6- naphthyridin-6(5H)-yl)-4H- pyrimido[1,2-b]pyridazin-4-one | 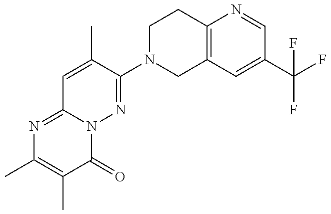 [SMILES: Cc1cc2nc(C)c(C)c(=O)n2nc1N1CCc2ncc(C(F)(F)F)cc2C1] | 390.2 |
| 1-26 | 2-(difluoromethyl)-8-methyl-7-(3- (trifluoromethyl)-7,8-dihydro-1,6- naphthyridin-6(5H)-yl)-4H- pyrimido[1,2-b]pyridazin-4-one | 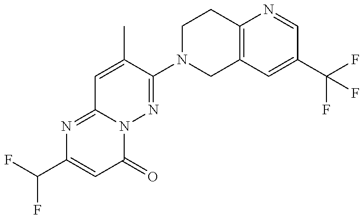 [SMILES: Cc1cc2nc(C(F)F)cc(=O)n2nc1N1CCc2ncc(C(F)(F)F)cc2C1] | 412 |
| 1-27 | 2-cyclopropyl-8-methyl-7-(3- (trifluoromethyl)-7,8-dihydro-1,6- naphthyridin-6(5H)-yl)-4H- pyrimido[1,2-b]pyridazin-4-one | 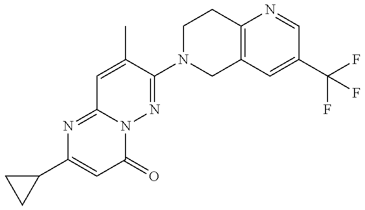 [SMILES: Cc1cc2nc(C3CC3)cc(=O)n2nc1N1CCc2ncc(C(F)(F)F)cc2C1] | 402.2 |
| 1-28 | 3-chloro-2,8-dimethyl-7-(3- (trifluoromethyl)-7,8-dihydro-1,6- naphthyridin-6(5H)-yl)-4H- pyrimido[1,2-b]pyridazin-4-one | 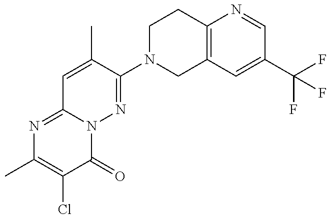 [SMILES: Cc1cc2nc(C)c(Cl)c(=O)n2nc1N1CCc2ncc(C(F)(F)F)cc2C1] | 410.2 |
| 1-29 | 2,8-dimethyl-7-(3-(1-methyl-1H- pyrazol-5-yl)-7,8-dihydro-1,6- naphthyridin-6(5H)-yl)-4H- pyrimido[1,2-b]pyridazin-4-one | 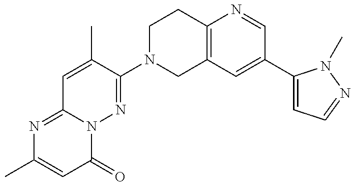 [SMILES: Cc1cc(=O)n2nc(N3CCc4ncc(-c5ccnn5C)cc4C3)c(C)cc2n1] | 388.3 |
| 1-30 | 2,8-dimethyl-7-(3-(6- methylpyridin-3-yl)-7,8-dihydro- 1,6-naphthyridin-6(5H)-yl)-4H- pyrimido[1,2-b]pyridazin-4-one | 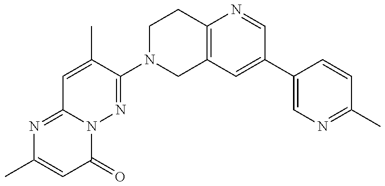 | 399.2 |
| 1-31 | 2,8-dimethyl-7-(3-(2-methylthiazol- 5-yl)-7,8-dihydro-1,6- naphthyridin-6(5H)-yl)-4H- pyrimido[1,2-b]pyridazin-4-one | 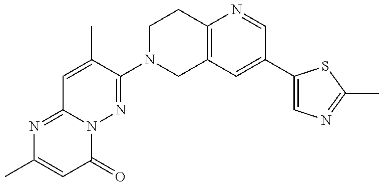 | 405.2 |
| 1-32 | 7-(3-(1-cyclopropyl-1H-pyrazol-4- yl)-7,8-dihydro-1,6-naphthyridin- 6(5H)-yl)-2,8-dimethyl-4H- pyrimido[1,2-b]pyridazin-4-one | 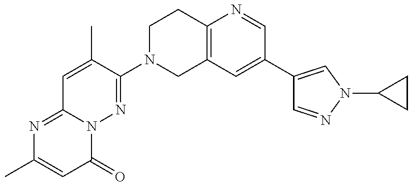 [SMILES: Cc1cc(=O)n2nc(N3CCc4ncc(-c5cnn(C6CC6)c5)cc4C3)c(C)cc2n1] | 414.2 |
| 1-33 | 2,8,9-trimethyl-7-(3- (trifluoromethyl)-7,8-dihydro-1,6- naphthyridin-6(5H)-yl)-4H- pyrimido[1,2-b]pyridazin-4-one | 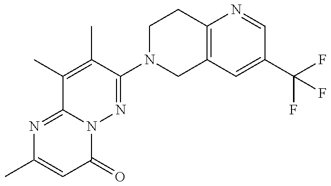 [SMILES: Cc1cc(=O)n2nc(N3CCc4ncc(C(F)(F)F)cc4C3)c(C)c(C)c2n1] | 390.2 |
| 1-34 | 2-(difluoromethyl)-7-(3-(1,3- dimethyl-1H-pyrazol-4-yl)-7,8- dihydro-1,6-naphthyridin-6(5H)- yl)-8-methyl-4H-pyrimido[1,2- b]pyridazin-4-one | 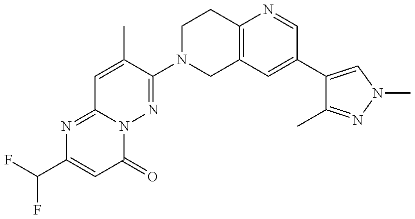 [SMILES: Cc1cc2nc(C(F)F)cc(=O)n2nc1N1CCc2ncc(-c3cn(C)nc3C)cc2C1] | 438.2 |
| 1-35 | 2-(difluoromethyl)-8-methyl-7-(3- (1-methyl-1H-pyrazol-4-yl)-7,8- dihydro-1,6-naphthyridin-6(5H)- yl)-4H-pyrimido[1,2-b]pyridazin-4- one | 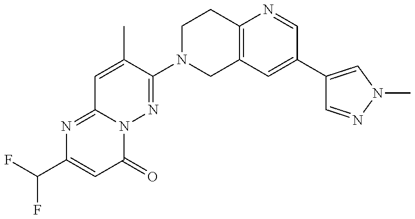 [SMILES: Cc1cc2nc(C(F)F)cc(=O)n2nc1N1CCc2ncc(-c3cnn(C)c3)cc2C1] | 424.2 |
| 1-36 | 2-(difluoromethyl)-8-methyl-7-(3- (1-methyl-1H-pyrazol-5-yl)-7,8- dihydro-1,6-naphthyridin-6(5H)- yl)-4H-pyrimido[1,2-b]pyridazin-4- one | 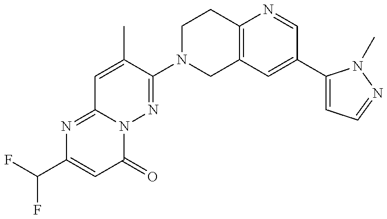 [SMILES: Cc1cc2nc(C(F)F)cc(=O)n2nc1N1CCc2ncc(-c3ccnn3C)cc2C1] | 424 |
| 1-37 | 8-methyl-7-(3-(1-methyl-2-oxo-1,2- dihydropyridin-4-yl)-7,8-dihydro- 1,6-naphthyridin-6(5H)-yl)-4H- pyrimido[1,2-b]pyridazin-4-one | 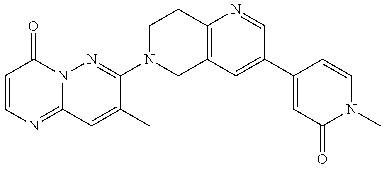 [SMILES: Cc1cc2nccc(=O)n2nc1N1CCc2ncc(-c3ccn(C)c(=O)c3)cc2C1] | 401.2 |
| 1-38 | 7-(3-(2-methoxypyridin-4-yl)-7,8- dihydro-1,6-naphthyridin-6(5H)- yl)-8-methyl-4H-pyrimido[1,2- b]pyridazin-4-one | 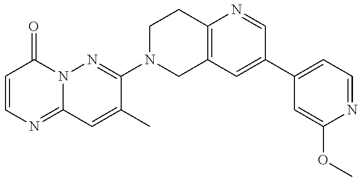 [SMILES: COc1cc(-c2cnc3c(c2)CN(c2nn4c(=O)ccnc4cc2C)CC3)ccn1] | 401.2 |
| 1-39 | 8,9-dimethyl-2-(trifluoromethyl)-7- (3-(trifluoromethyl)-7,8-dihydro- 1,6-naphthyridin-6(5H)-yl)-4H- pyrimido[1,2-b]pyridazin-4-one | 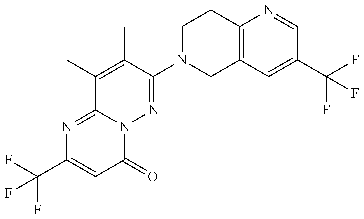 [SMILES: Cc1c(N2CCc3ncc(C(F)(F)F)cc3C2)nn2c(=O)cc(C(F)(F)F)nc2c1C] | 444 |
| 1-40 | 2-(difluoromethyl)-8,9-dimethyl-7- (3-(trifluoromethyl)-7,8-dihydro- 1,6-naphthyridin-6(5H)-yl)-4H- pyrimido[1,2-b]pyridazin-4-one | 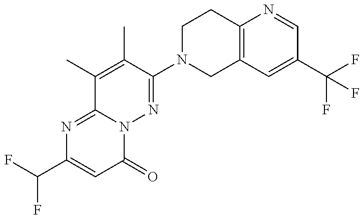 [SMILES: Cc1c(N2CCc3ncc(C(F)(F)F)cc3C2)nn2c(=O)cc(C(F)F)nc2c1C] | 426.2 |
| 1-41 | 8-methyl-7-(3-(quinolin-5-yl)-7,8- dihydro-1,6-naphthyridin-6(5H)- yl)-4H-pyrimido[1,2-b]pyridazin-4- one | 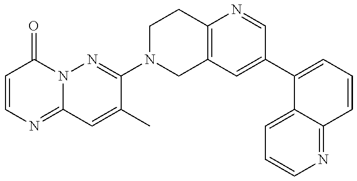 [SMILES: Cc1cc2nccc(=O)n2nc1N1CCc2ncc(-c3cccc4ncccc34)cc2C1] | 421.2 |
| 1-42 | 8-methyl-4-oxo-N-(thiazol-5- ylmethyl)-7-(3-(trifluoromethyl)- 7,8-dihydro-1,6-naphthyridin- 6(5H)-yl)-4H-pyrimido[1,2- b]pyridazine-2-carboxamide | 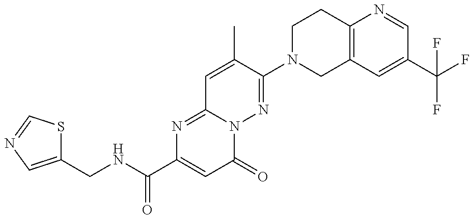 [SMILES: Cc1cc2nc(C(=O)NCc3cncs3)cc(=O)n2nc1N1CCc2ncc(C(F)(F)F)cc2C1] | 502.3 |
| 1-43 | 8-methyl-2-(3-(pyridin-3- yl)pyrrolidine-1-carbonyl)-7-(3- (trifluoromethyl)-7,8-dihydro-1,6- naphthyridin-6(5H)-yl)-4H- pyrimido[1,2-b]pyridazin-4-one | 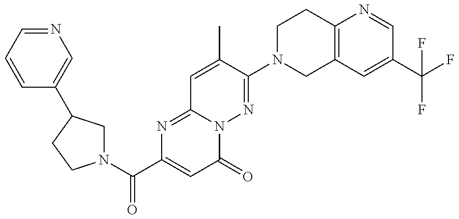 [SMILES: Cc1cc2nc(C(=O)N3CCC(c4cccnc4)C3)cc(=O)n2nc1N1CCc2ncc(C(F)(F)F)cc2C1] | 536.3 |
| 1-44 | 8-methyl-2-(2-oxa-6- azaspiro[3.3]heptane-6-carbonyl)-7- (3-(trifluoromethyl)-7,8-dihydro- 1,6-naphthyridin-6(5H)-yl)-4H- pyrimido[1,2-b]pyridazin-4-one | 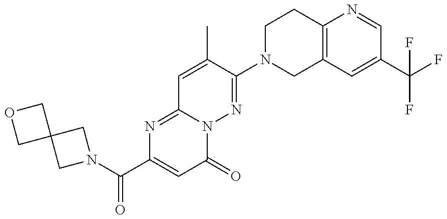 [SMILES: Cc1cc2nc(C(=O)N3CC4(COC4)C3)cc(=O)n2nc1N1CCc2ncc(C(F)(F)F)cc2C1] | 487.2 |
| 1-45 | 2-(3-fluoro-3-methylazetidine-1- carbonyl)-8-methyl-7-(3- (trifluoromethyl)-7,8-dihydro-1,6- naphthyridin-6(5H)-yl)-4H- pyrimido[1,2-b]pyridazin-4-one | 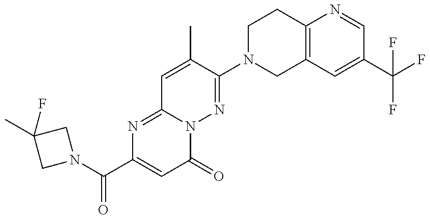 [SMILES: Cc1cc2nc(C(=O)N3CC(C)(F)C3)cc(=O)n2nc1N1CCc2ncc(C(F)(F)F)cc2C1] | 477.2 |
| 1-46 | N-(1-cyanocyclopropyl)-8-methyl- 4-oxo-7-(3-(trifluoromethyl)-7,8- dihydro-1,6-naphthyridin-6(5H)- yl)-4H-pyrimido[1,2-b]pyridazine- 2-carboxamide | 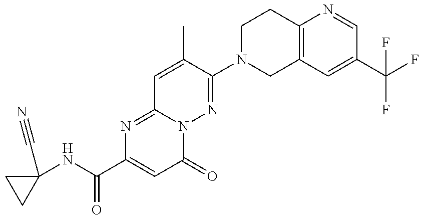 [SMILES: Cc1cc2nc(C(=O)NC3(C#N)CC3)cc(=O)n2nc1N1CCc2ncc(C(F)(F)F)cc2C1] | 470.2 |
| 1-47 | 2-(3-methoxyazetidine-1-carbonyl)- 8-methyl-7-(3-(trifluoromethyl)- 7,8-dihydro-1,6-naphthyridin- 6(5H)-yl)-4H-pyrimido[1,2- b]pyridazin-4-one | 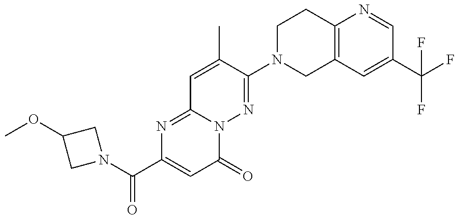 [SMILES: COC1CN(C(=O)c2cc(=O)n3nc(N4CCc5ncc(C(F)(F)F)cc5C4)c(C)cc3n2)C1] | 475.3 |
| 1-48 | 8-methyl-7-(3-(2-methylquinolin-5- yl)-7,8-dihydro-1,6-naphthyridin- 6(5H)-yl)-4H-pyrimido[1,2- b]pyridazin-4-one | 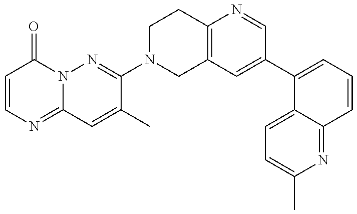 [SMILES: Cc1ccc2c(-c3cnc4c(c3)CN(c3nn5c(=O)ccnc5cc3C)CC4)cccc2n1] | 436 |
| 1-49 | 8-methyl-7-(3-(1-methyl-6-oxo-1,6- dihydropyridin-3-yl)-7,8-dihydro- 1,6-naphthyridin-6(5H)-yl)-4H- pyrimido[1,2-b]pyridazin-4-one | 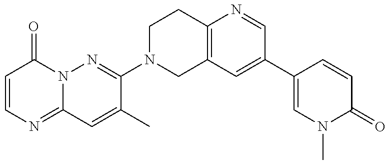 [SMILES: Cc1cc2nccc(=O)n2nc1N1CCc2ncc(-c3ccc(=O)n(C)c3)cc2C1] | 401.2 |
| 1-50 | 7-(3-(3-methoxypyridin-4-yl)-7,8- dihydro-1,6-naphthyridin-6(5H)- yl)-8-methyl-4H-pyrimido[1,2- b]pyridazin-4-one | 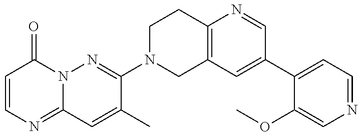 [SMILES: COc1cnccc1-c1cnc2c(c1)CN(c1nn3c(=O)ccnc3cc1C)CC2] | 401.2 |
| 1-51 | 7-(3-(4-fluoro-3-methoxyphenyl)- 7,8-dihydro-1,6-naphthyridin- 6(5H)-yl)-8-methyl-4H- pyrimido[1,2-b]pyridazin-4-one | 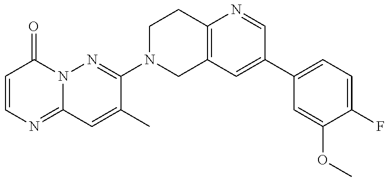 [SMILES: COc1cc(-c2cnc3c(c2)CN(c2nn4c(=O)ccnc4cc2C)CC3)ccc1F] | 418.2 |
| 1-52 | 7-(3-(5-methoxypyridin-3-yl)-7,8- dihydro-1,6-naphthyridin-6(5H)- yl)-8-methyl-4H-pyrimido[1,2- b]pyridazin-4-one | 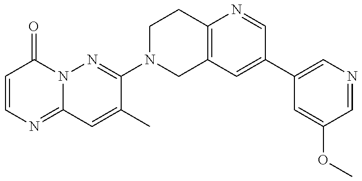 [SMILES: COc1cncc(-c2cnc3c(c2)CN(c2nn4c(=O)ccnc4cc2C)CC3)c1] | 401.2 |
| 1-53 | 7-(3-(imidazo[1,2-a]pyridin-6-yl)- 7,8-dihydro-1,6-naphthyridin- 6(5H)-yl)-8-methyl-4H- pyrimido[1,2-b]pyridazin-4-one | 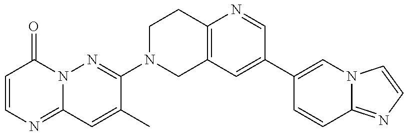 [SMILES: Cc1cc2nccc(=O)n2nc1N1CCc2ncc(-c3ccc4nccn4c3)cc2C1] | 411.2 |
| 1-54 | 7-(3-(2-cyclopropylpyridin-4-yl)- 7,8-dihydro-1,6-naphthyridin- 6(5H)-yl)-8-methyl-4H- pyrimido[1,2-b]pyridazin-4-one | 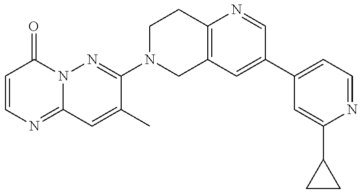 [SMILES: Cc1cc2nccc(=O)n2nc1N1CCc2ncc(-c3ccnc(C4CC4)c3)cc2C1] | 411.3 |
| 1-55 | 8-methyl-4-oxo-N-(1-(2,2,2- trifluoroethyl)azetidin-3-yl)-7-(3- (trifluoromethyl)-7,8-dihydro-1,6- naphthyridin-6(5H)-yl)-4H- pyrimido[1,2-b]pyridazine-2- carboxamide | 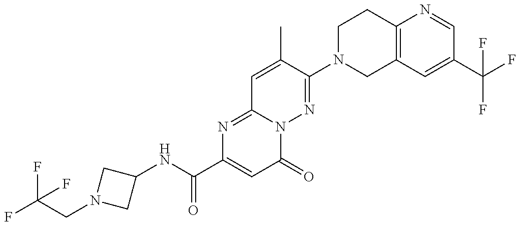 [SMILES: Cc1cc2nc(C(=O)NC3CN(CC(F)(F)F)C3)cc(=O)n2nc1N1CCc2ncc(C(F)(F)F)cc2C1] | 542.2 |
| 1-56 | 8-methyl-2-(3-methylazetidine-1- carbonyl)-7-(3-(trifluoromethyl)- 7,8-dihydro-1,6-naphthyridin- 6(5H)-yl)-4H-pyrimido[1,2- b]pyridazin-4-one | 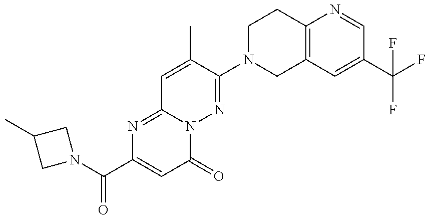 [SMILES: Cc1cc2nc(C(=O)N3CC(C)C3)cc(=O)n2nc1N1CCc2ncc(C(F)(F)F)cc2C1] | 459.4 |
| 1-57 | 7-(3-(5-chloro-2-methoxypyridin-4- yl)-7,8-dihydro-1,6-naphthyridin- 6(5H)-yl)-8-methyl-4H- pyrimido[1,2-b]pyridazin-4-one | 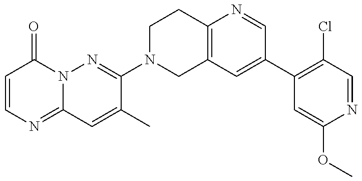 [SMILES: COc1cc(-c2cnc3c(c2)CN(c2nn4c(=O)ccnc4cc2C)CC3)c(Cl)cn1] | 435.4 |
| 1-58 | 7-(3-(2-fluoropyridin-3-yl)-7,8- dihydro-1,6-naphthyridin-6(5H)- yl)-8-methyl-4H-pyrimido[1,2- b]pyridazin-4-one | 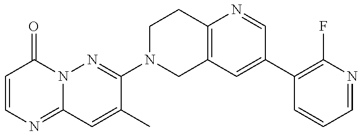 [SMILES: Cc1cc2nccc(=O)n2nc1N1CCc2ncc(-c3cccnc3F)cc2C1] | 389.2 |
| 1-59 | 7-(3-(3-chloro-4-fluorophenyl)-7,8- dihydro-1,6-naphthyridin-6(5H)- yl)-8-methyl-4H-pyrimido[1,2- b]pyridazin-4-one | 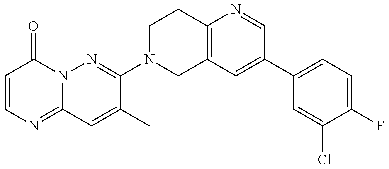 [SMILES: Cc1cc2nccc(=O)n2nc1N1CCc2ncc(-c3ccc(F)c(Cl)c3)cc2C1] | 422.2 |
| 1-60 | 7-(3-(6-methoxypyridin-3-yl)-7,8- dihydro-1,6-naphthyridin-6(5H)- yl)-8-methyl-4H-pyrimido[1,2- b]pyridazin-4-one | 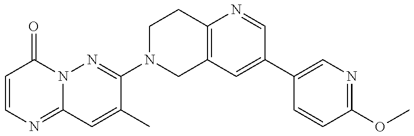 [SMILES: COc1ccc(-c2cnc3c(c2)CN(c2nn4c(=O)ccnc4cc2C)CC3)cn1] | 401.4 |
| 1-61 | 8,9-dimethyl-7-(3-(6- methylpyridin-3-yl)-7,8-dihydro- 1,6-naphthyridin-6(5H)-yl)-4H- pyrimido[1,2-b]pyridazin-4-one | 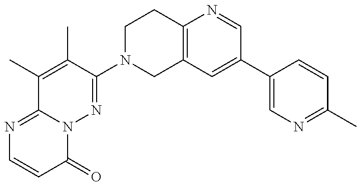 [SMILES: Cc1ccc(-c2cnc3c(c2)CN(c2nn4c(=O)ccnc4c(C)c2C)CC3)cn1] | 399.2 |
| 1-62 | 8,9-dimethyl-7-(3-(1-methyl-1H- pyrazol-4-yl)-7,8-dihydro-1,6- naphthyridin-6(5H)-yl)-4H- pyrimido[1,2-b]pyridazin-4-one | 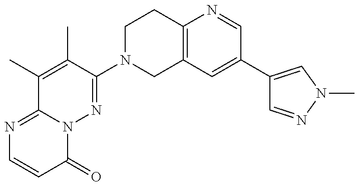 [SMILES: Cc1c(N2CCc3ncc(-c4cnn(C)c4)cc3C2)nn2c(=O)ccnc2c1C] | 388.2 |
| 1-63 | 8,9-dimethyl-7-(3-(1-methyl-1H- pyrazol-5-yl)-7,8-dihydro-1,6- naphthyridin-6(5H)-yl)-4H- pyrimido[1,2-b]pyridazin-4-one | 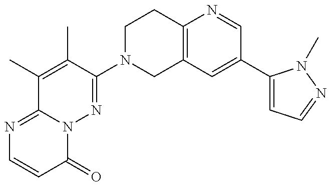 [SMILES: Cc1c(N2CCc3ncc(-c4ccnn4C)cc3C2)nn2c(=O)ccnc2c1C] | 388.4 |
| 1-64 | 8,9-dimethyl-7-(3- (trifluoromethyl)-7,8-dihydro-1,6- naphthyridin-6(5H)-yl)-4H- pyrimido[1,2-b]pyridazin-4-one | 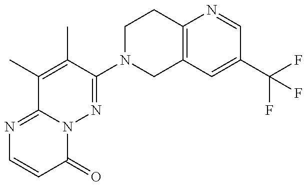 [SMILES: Cc1c(N2CCc3ncc(C(F)(F)F)cc3C2)nn2c(=O)ccnc2c1C] | 376.2 |
| 1-65 | 8-methyl-7-(3-(5-methylpyridin-3- yl)-7,8-dihydro-1,6-naphthyridin- 6(5H)-yl)-4H-pyrimido[1,2- b]pyridazin-4-one | 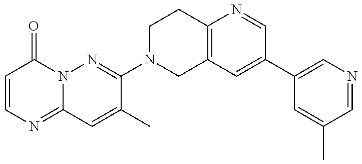 [SMILES: Cc1cncc(-c2cnc3c(c2)CN(c2nn4c(=O)ccnc4cc2C)CC3)c1] | 385.4 |
| 1-66 | 8-methyl-7-(3-(6- (trifluoromethyl)pyridin-3-yl)-7,8- dihydro-1,6-naphthyridin-6(5H)- yl)-4H-pyrimido[1,2-b]pyridazin-4- one | 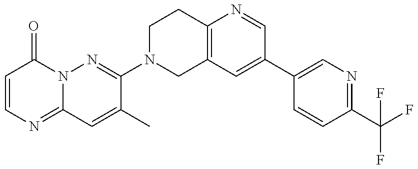 [SMILES: Cc1cc2nccc(=O)n2nc1N1CCc2ncc(-c3ccc(C(F)(F)F)nc3)cc2C1] | 385.4 |
| 1-67 | 7-(3-(6-cyclopropylpyridin-3-yl)- 7,8-dihydro-1,6-naphthyridin- 6(5H)-yl)-8-methyl-4H- pyrimido[1,2-b]pyridazin-4-one | 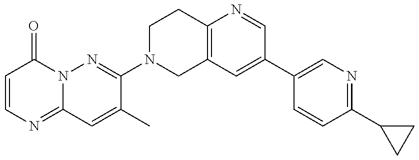 [SMILES: Cc1cc2nccc(=O)n2nc1N1CCc2ncc(-c3ccc(C4CC4)nc3)cc2C1] | 411.4 |
| 1-68 | 8-methyl-7-(3-(pyridin-3-yl)-7,8- dihydro-1,6-naphthyridin-6(5H)- yl)-4H-pyrimido[1,2-b]pyridazin-4- one | 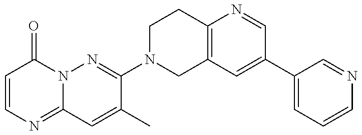 [SMILES: Cc1cc2nccc(=O)n2nc1N1CCc2ncc(-c3cccnc3)cc2C1] | 371.4 |
| 1-69 | 7-(3-(2-fluoropyridin-3-yl)-7,8- dihydro-1,6-naphthyridin-6(5H)- yl)-2,8-dimethyl-4H-pyrimido[1,2- b]pyridazin-4-one | 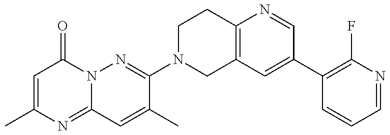 [SMILES: Cc1cc(=O)n2nc(N3CCc4ncc(-c5cccnc5F)cc4C3)c(C)cc2n1] | 403.2 |
| 1-70 | 7-(3-(5-methoxypyridin-3-yl)-7,8- dihydro-1,6-naphthyridin-6(5H)- yl)-2,8-dimethyl-4H-pyrimido[1,2- b]pyridazin-4-one | 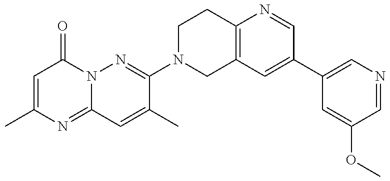 | 416.2 |
| 1-71 | 7-(3-(6-methoxypyridin-3-yl)-7,8- dihydro-1,6-naphthyridin-6(5H)- yl)-2,8-dimethyl-4H-pyrimido[1,2- b]pyridazin-4-one | 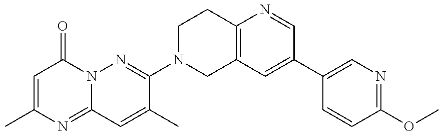 | 415.2 |
| 1-72 | 7-(3-(6-cyclopropylpyridin-3-yl)- 7,8-dihydro-1,6-naphthyridin- 6(5H)-yl)-2,8-dimethyl-4H- pyrimido[1,2-b]pyridazin-4-one | 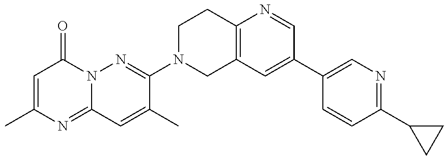 [SMILES: Cc1cc(=O)n2nc(N3CCc4ncc(-c5ccc(C6CC6)nc5)cc4C3)c(C)cc2n1] | 425.2 |
| 1-73 | 2,8-dimethyl-7-(3-(6- (trifluoromethyl)pyridin-3-yl)-7,8- dihydro-1,6-naphthyridin-6(5H)- yl)-4H-pyrimido[1,2-b]pyridazin-4- one | 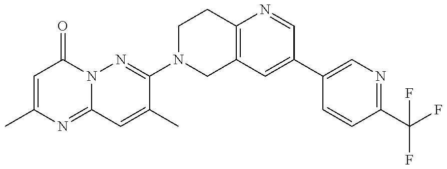 [SMILES: Cc1cc(=O)n2nc(N3CCc4ncc(-c5ccc(C(F)(F)F)nc5)cc4C3)c(C)cc2n1] | 453.2 |
| 1-74 | 7-(3-(5-methoxypyridin-3-yl)-7,8- dihydro-1,6-naphthyridin-6(5H)- yl)-8-methyl-2-(trifluoromethyl)- 4H-pyrimido[1,2-b]pyridazin-4-one | 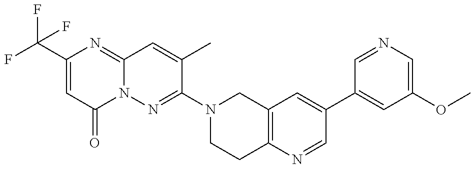 [SMILES: COc1cncc(-c2cnc3c(c2)CN(c2nn4c(=O)cc(C(F)(F)F)nc4cc2C)CC3)c1] | 469.2 |
| 1-75 | 7-(3-(6-methoxypyridin-3-yl)-7,8- dihydro-1,6-naphthyridin-6(5H)- yl)-8-methyl-2-(trifluoromethyl)- 4H-pyrimido[1,2-b]pyridazin-4-one | 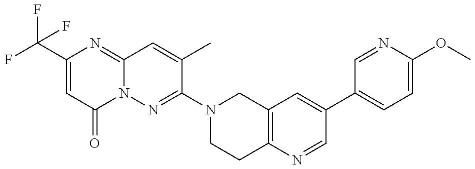 [SMILES: COc1ccc(-c2cnc3c(c2)CN(c2nn4c(=O)cc(C(F)(F)F)nc4cc2C)CC3)cn1] | 469.2 |
| 1-76 | N-(1-cyanocyclopropyl)-8-methyl- 7-(3-(6-methylpyridin-3-yl)-7,8- dihydro-1,6-naphthyridin-6(5H)- yl)-4-oxo-4H-pyrimido[1,2- b]pyridazine-2-carboxamide | 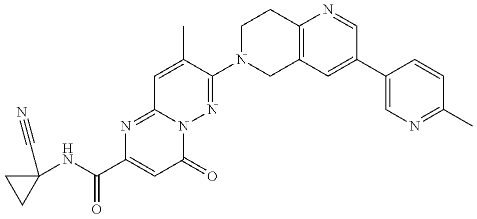 [SMILES: Cc1ccc(-c2cnc3c(c2)CN(c2nn4c(=O)cc(C(=O)NC5(C#N)CC5)nc4cc2C)CC3)cn1] | 493.2 |
| 1-77 | 8-methyl-7-(3-(6-methylpyridin-3- yl)-7,8-dihydro-1,6-naphthyridin- 6(5H)-yl)-4-oxo-N-(thiazol-5- ylmethyl)-4H-pyrimido[1,2- b]pyridazine-2-carboxamide | 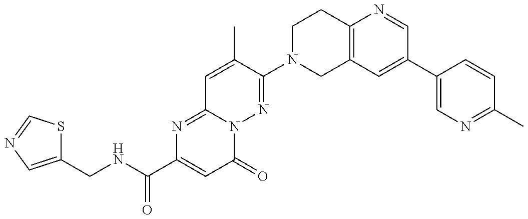 [SMILES: Cc1ccc(-c2cnc3c(c2)CN(c2nn4c(=O)cc(C(=O)NCc5cncs5)nc4cc2C)CC3)cn1] | 525.1 |
| 1-78 | 3-bromo-8-methyl-7-(3-(6- methylpyridin-3-yl)-7,8-dihydro- 1,6-naphthyridin-6(5H)-yl)-4H- pyrimido[1,2-b]pyridazin-4-one | 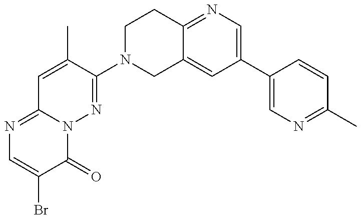 [SMILES: Cc1ccc(-c2cnc3c(c2)CN(c2nn4c(=O)c(Br)cnc4cc2C)CC3)cn1] | 463.2 |
| 1-79 | 2-cyclopropyl-8-methyl-7-(3-(6- methylpyridin-3-yl)-7,8-dihydro- 1,6-naphthyridin-6(5H)-yl)-4H- pyrimido[1,2-b]pyridazin-4-one | 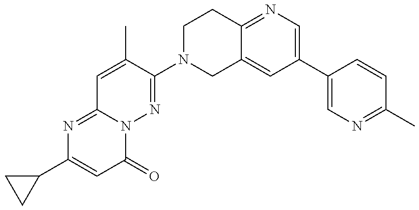 [SMILES: Cc1ccc(-c2cnc3c(c2)CN(c2nn4c(=O)cc(C5CC5)nc4cc2C)CC3)cn1] | 425.4 |
| 1-80 | 8-methyl-7-(3-(6-methylpyridin-3- yl)-7,8-dihydro-1,6-naphthyridin- 6(5H)-yl)-2-(trifluoromethyl)-4H- pyrimido[1,2-b]pyridazin-4-one | 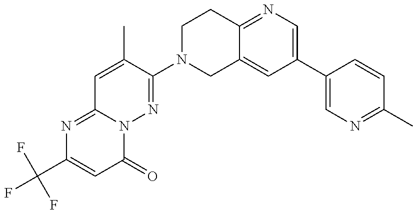 [SMILES: Cc1ccc(-c2cnc3c(c2)CN(c2nn4c(=O)cc(C(F)(F)F)nc4cc2C)CC3)cn1] | 453.3 |
| 1-81 | 2-(difluoromethyl)-8-methyl-7-(3- (6-methylpyridin-3-yl)-7,8-dihydro- 1,6-naphthyridin-6(5H)-yl)-4H- pyrimido[1,2-b]pyridazin-4-one | 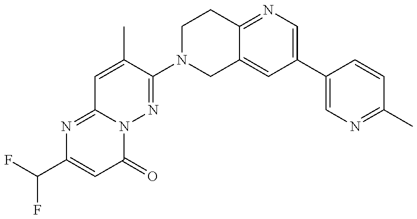 [SMILES: Cc1ccc(-c2cnc3c(c2)CN(c2nn4c(=O)cc(C(F)F)nc4cc2C)CC3)cn1] | 435.4 |
| 1-82 | 2,8,9-trimethyl-7-(3-(6- methylpyridin-3-yl)-7,8-dihydro- 1,6-naphthyridin-6(5H)-yl)-4H- pyrimido[1,2-b]pyridazin-4-one | 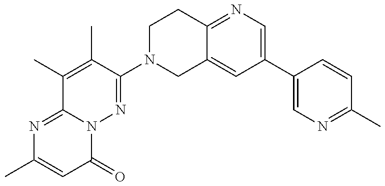 | 413.3 |
| 1-83 | 2,3,8-trimethyl-7-(3-(6- methylpyridin-3-yl)-7,8-dihydro- 1,6-naphthyridin-6(5H)-yl)-4H- pyrimido[1,2-b]pyridazin-4-one | 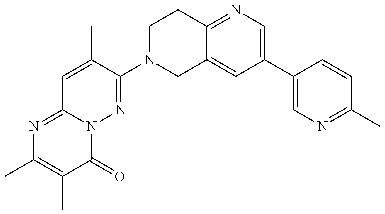 | 413.4 |
| 1-84 | 8,9-dimethyl-7-(3-(6- methylpyridin-3-yl)-7,8-dihydro- 1,6-naphthyridin-6(5H)-yl)-2- (trifluoromethyl)-4H-pyrimido[1,2- b]pyridazin-4-one | 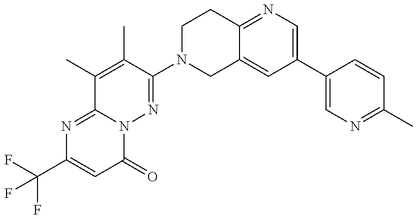 [SMILES: Cc1ccc(-c2cnc3c(c2)CN(c2nn4c(=O)cc(C(F)(F)F)nc4c(C)c2C)CC3)cn1] | 467.2 |
| 1-85 | 2-(difluoromethyl)-8,9-dimethyl-7- (3-(6-methylpyridin-3-yl)-7,8- dihydro-1,6-naphthyridin-6(5H)- yl)-4H-pyrimido[1,2-b]pyridazin-4- one | 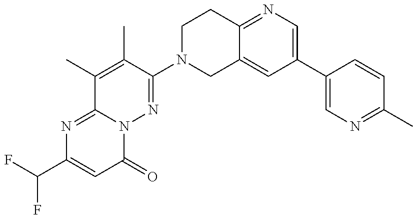 [SMILES: Cc1ccc(-c2cnc3c(c2)CN(c2nn4c(=O)cc(C(F)F)nc4c(C)c2C)CC3)cn1] | 449.2 |
| 1-86 | 3,8-dimethyl-7-(3-(6- methylpyridin-3-yl)-7,8-dihydro- 1,6-naphthyridin-6(5H)-yl)-4H- pyrimido[1,2-b]pyridazin-4-one | 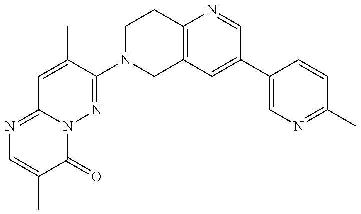 [SMILES: Cc1ccc(-c2cnc3c(c2)CN(c2nn4c(=O)c(C)cnc4cc2C)CC3)cn1] | 399.4 |
| 1-87 | 7-(3-(6-fluoropyridin-3-yl)-7,8- dihydro-1,6-naphthyridin-6(5H)- yl)-8,9-dimethyl-4H-pyrimido[1,2- b]pyridazin-4-one | 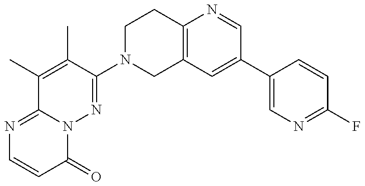 [SMILES: Cc1c(N2CCc3ncc(-c4ccc(F)nc4)cc3C2)nn2c(=O)ccnc2c1C] | 403.2 |
| 1-88 | 3-bromo-8,9-dimethyl-7-(3-(6- methylpyridin-3-yl)-7,8-dihydro- 1,6-naphthyridin-6(5H)-yl)-4H- pyrimido[1,2-b]pyridazin-4-one | 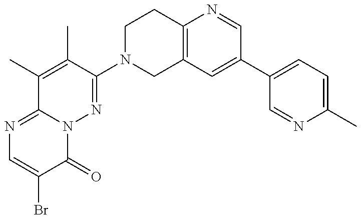 [SMILES: Cc1ccc(-c2cnc3c(c2)CN(c2nn4c(=O)c(Br)cnc4c(C)c2C)CC3)cn1] | 477 |
| 1-89 | 7-(3-(6-(dimethylamino)pyridin-3- yl)-7,8-dihydro-1,6-naphthyridin- 6(5H)-yl)-8,9-dimethyl-4H- pyrimido[1,2-b]pyridazin-4-one | 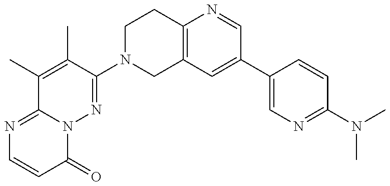 [SMILES: Cc1c(N2CCc3ncc(-c4ccc(N(C)C)nc4)cc3C2)nn2c(=O)ccnc2c1C] | 428.5 |
| 1-90 | 8,9-dimethyl-7-(3-(6- (trifluoromethyl)pyridin-3-yl)-7,8- dihydro-1,6-naphthyridin-6(5H)- yl)-4H-pyrimido[1,2-b]pyridazin-4- one | 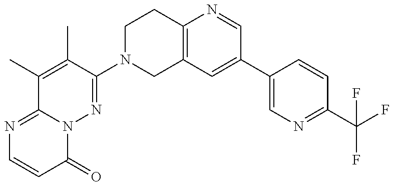 [SMILES: Cc1c(N2CCc3ncc(-c4ccc(C(F)(F)F)nc4)cc3C2)nn2c(=O)ccnc2c1C] | 453.3 |
| 1-91 | 7-(3-(6-cyclopropylpyridin-3-yl)- 7,8-dihydro-1,6-naphthyridin- 6(5H)-yl)-8,9-dimethyl-4H- pyrimido[1,2-b]pyridazin-4-one | 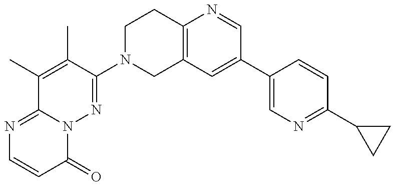 [SMILES: Cc1c(N2CCc3ncc(-c4ccc(C5CC5)nc4)cc3C2)nn2c(=O)ccnc2c1C] | 425.4 |
| 1-92 | 2-(difluoromethyl)-7-(3-(6- (dimethylamino)pyridin-3-yl)-7,8- dihydro-1,6-naphthyridin-6(5H)- yl)-8,9-dimethyl-4H-pyrimido[1,2- b]pyridazin-4-one | 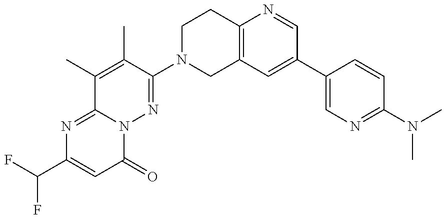 [SMILES: Cc1c(N2CCc3ncc(-c4ccc(N(C)C)nc4)cc3C2)nn2c(=O)cc(C(F)F)nc2c1C] | 478.2 |
| 1-93 | 2-(difluoromethyl)-8,9-dimethyl-7- (3-(6-(trifluoromethyl)pyridin-3- yl)-7,8-dihydro-1,6-naphthyridin- 6(5H)-yl)-4H-pyrimido[1,2- b]pyridazin-4-one | 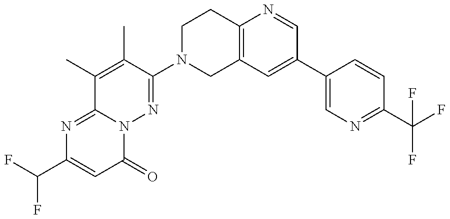 [SMILES: Cc1c(N2CCc3ncc(-c4ccc(C(F)(F)F)nc4)cc3C2)nn2c(=O)cc(C(F)F)nc2c1C] | 503 |
| 1-94 | 7-(3-(6-cyclopropylpyridin-3-yl)- 7,8-dihydro-1,6-naphthyridin- 6(5H)-yl)-2-(difluoromethyl)-8,9- dimethyl-4H-pyrimido[1,2- b]pyridazin-4-one | 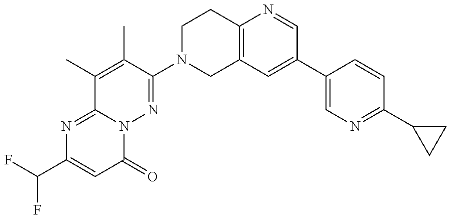 [SMILES: Cc1c(N2CCc3ncc(-c4ccc(C5CC5)nc4)cc3C2)nn2c(=O)cc(C(F)F)nc2c1C] | 475.2 |
| 1-95 | 8,9-dimethyl-7-(3-(4- methylpyridin-3-yl)-7,8-dihydro- 1,6-naphthyridin-6(5H)-yl)-4H- pyrimido[1,2-b]pyridazin-4-one | 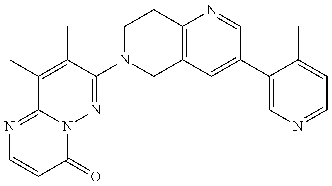 [SMILES: Cc1ccncc1-c1cnc2c(c1)CN(c1nn3c(=O)ccnc3c(C)c1C)CC2] | 399.4 |
| 1-96 | 7-(3-(4-ethylpyridin-3-yl)-7,8- dihydro-1,6-naphthyridin-6(5H)- yl)-8,9-dimethyl-4H-pyrimido[1,2- b]pyridazin-4-one | 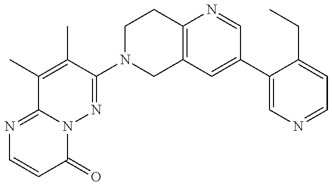 [SMILES: CCc1ccncc1-c1cnc2c(c1)CN(c1nn3c(=O)ccnc3c(C)c1C)CC2] | 413.4 |
| 1-97 | 8,9-dimethyl-7-(3-(6-(piperidin-1- yl)pyridin-3-yl)-7,8-dihydro-1,6- naphthyridin-6(5H)-yl)-4H- pyrimido[1,2-b]pyridazin-4-one | 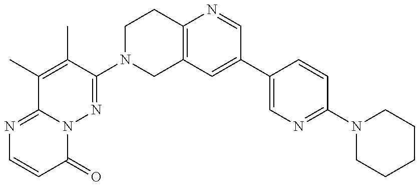 [SMILES: Cc1c(N2CCc3ncc(-c4ccc(N5CCCCC5)nc4)cc3C2)nn2c(=O)ccnc2c1C] | 468.4 |
| 1-98 | 7-(3-(6-(1H-imidazol-1-yl)pyridin- 3-yl)-7,8-dihydro-1,6-naphthyridin- 6(5H)-yl)-8,9-dimethyl-4H- pyrimido[1,2-b]pyridazin-4-one | 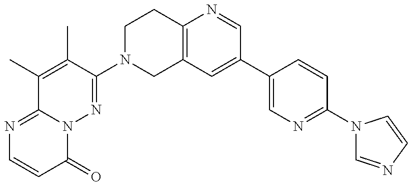 [SMILES: Cc1c(N2CCc3ncc(-c4ccc(-n5ccnc5)nc4)cc3C2)nn2c(=O)ccnc2c1C] | 451.4 |
| 1-99 | 8,9-dimethyl-7-(3-(6- morpholinopyridin-3-yl)-7,8- dihydro-1,6-naphthyridin-6(5H)- yl)-4H-pyrimido[1,2-b]pyridazin-4- one | 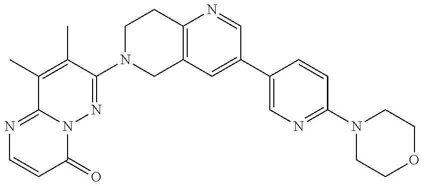 [SMILES: Cc1c(N2CCc3ncc(-c4ccc(N5CCOCC5)nc4)cc3C2)nn2c(=O)ccnc2c1C] | 470.4 |
| 1-100 | 3,8,9-trimethyl-7-(3-(6- methylpyridin-3-yl)-7,8-dihydro- 1,6-naphthyridin-6(5H)-yl)-4H- pyrimido[1,2-b]pyridazin-4-one | 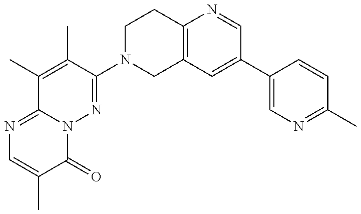 [SMILES: Cc1ccc(-c2cnc3c(c2)CN(c2nn4c(=O)c(C)cnc4c(C)c2C)CC3)cn1] | 413.4 |
| 1-101 | 8,9-dimethyl-7-(3-(2- methylpyridin-3-yl)-7,8-dihydro- 1,6-naphthyridin-6(5H)-yl)-4H- pyrimido[1,2-b]pyridazin-4-one | 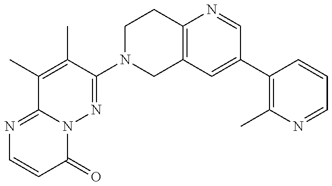 [SMILES: Cc1ncccc1-c1cnc2c(c1)CN(c1nn3c(=O)ccnc3c(C)c1C)CC2] | 399.2 |
| 1-102 | 8,9-dimethyl-7-(3-(5- (trifluoromethyl)pyridin-3-yl)-7,8- dihydro-1,6-naphthyridin-6(5H)- yl)-4H-pyrimido[1,2-b]pyridazin-4- one | 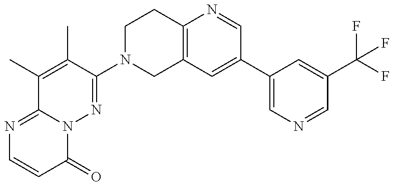 [SMILES: Cc1c(N2CCc3ncc(-c4cncc(C(F)(F)F)c4)cc3C2)nn2c(=O)ccnc2c1C] | 453.2 |
| 1-103 | 7-(3-(5-fluoropyridin-3-yl)-7,8- dihydro-1,6-naphthyridin-6(5H)- yl)-8,9-dimethyl-4H-pyrimido[1,2- b]pyridazin-4-one | 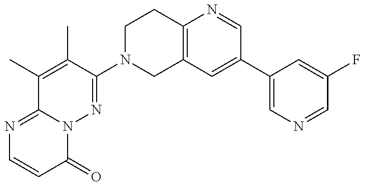 [SMILES: Cc1c(N2CCc3ncc(-c4cncc(F)c4)cc3C2)nn2c(=O)ccnc2c1C] | 403.2 |
| 1-104 | 8,9-dimethyl-7-(3-(pyrimidin-5-yl)- 7,8-dihydro-1,6-naphthyridin- 6(5H)-yl)-4H-pyrimido[1,2- b]pyridazin-4-one | 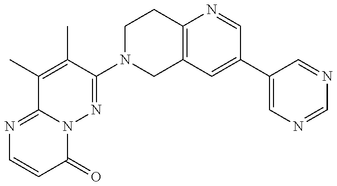 [SMILES: Cc1c(N2CCc3ncc(-c4cncnc4)cc3C2)nn2c(=O)ccnc2c1C] | 386.2 |
| 1-105 | 2-ethyl-8,9-dimethyl-7-(3- (trifluoromethyl)-7,8-dihydro-1,6- naphthyridin-6(5H)-yl)-4H- pyrimido[1,2-b]pyridazin-4-one | 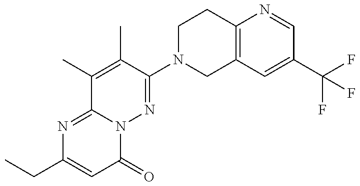 [SMILES: CCc1cc(=O)n2nc(N3CCc4ncc(C(F)(F)F)cc4C3)c(C)c(C)c2n1] | 404.2 |
| 1-106 | 2-ethyl-8,9-dimethyl-7-(3-(6- methylpyridin-3-yl)-7,8-dihydro- 1,6-naphthyridin-6(5H)-yl)-4H- pyrimido[1,2-b]pyridazin-4-one | 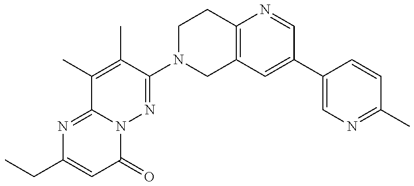 [SMILES: CCc1cc(=O)n2nc(N3CCc4ncc(-c5ccc(C)nc5)cc4C3)c(C)c(C)c2n1] | 427.2 |
| 1-107 | 2-isopropyl-8,9-dimethyl-7-(3- (trifluoromethyl)-7,8-dihydro-1,6- naphthyridin-6(5H)-yl)-4H- pyrimido[1,2-b]pyridazin-4-one | 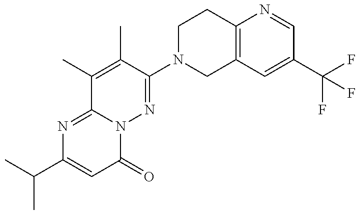 [SMILES: Cc1c(N2CCc3ncc(C(F)(F)F)cc3C2)nn2c(=O)cc(C(C)C)nc2c1C] | 418.2 |
| 1-108 | 2-isopropyl-8,9-dimethyl-7-(3-(6- methylpyridin-3-yl)-7,8-dihydro- 1,6-naphthyridin-6(5H)-yl)-4H- pyrimido[1,2-b]pyridazin-4-one | 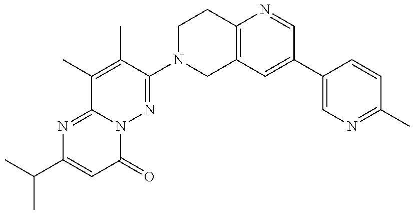 [SMILES: Cc1ccc(-c2cnc3c(c2)CN(c2nn4c(=O)cc(C(C)C)nc4c(C)c2C)CC3)cn1] | 441.2 |
| 1-109 | 2-(methoxymethyl)-8,9-dimethyl-7- (3-(trifluoromethyl)-7,8-dihydro- 1,6-naphthyridin-6(5H)-yl)-4H- pyrimido[1,2-b]pyridazin-4-one | 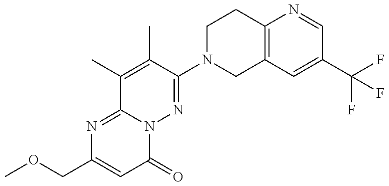 [SMILES: COCc1cc(=O)n2nc(N3CCc4ncc(C(F)(F)F)cc4C3)c(C)c(C)c2n1] | 420.2 |
| 1-110 | 2-(methoxymethyl)-8,9-dimethyl-7- (3-(6-methylpyridin-3-yl)-7,8- dihydro-1,6-naphthyridin-6(5H)- yl)-4H-pyrimido[1,2-b]pyridazin-4- one | 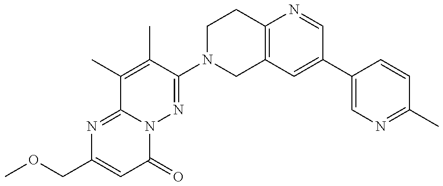 | 443.2 |
| 1-111 | 8-cyclopropyl-9-methyl-7-(3- (trifluoromethyl)-7,8-dihydro-1,6- naphthyridin-6(5H)-yl)-4H- pyrimido[1,2-b]pyridazin-4-one | 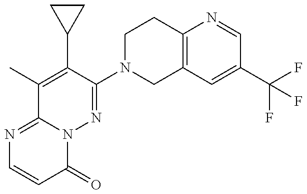 [SMILES: Cc1c(C2CC2)c(N2CCc3ncc(C(F)(F)F)cc3C2)nn2c(=O)ccnc12] | 402.2 |
| 1-112 | 8-cyclopropyl-9-methyl-7-(3-(6- methylpyridin-3-yl)-7,8-dihydro- 1,6-naphthyridin-6(5H)-yl)-4H- pyrimido[1,2-b]pyridazin-4-one | 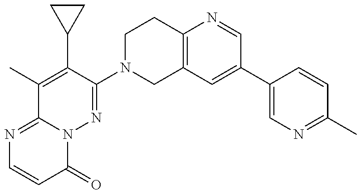 [SMILES: Cc1ccc(-c2cnc3c(c2)CN(c2nn4c(=O)ccnc4c(C)c2C2CC2)CC3)cn1] | 425.2 |
| 1-113 | 9-cyclopropyl-8-methyl-7-(3- (trifluoromethyl)-7,8-dihydro-1,6- naphthyridin-6(5H)-yl)-4H- pyrimido[1,2-b]pyridazin-4-one | 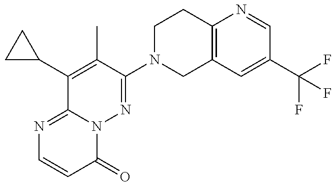 [SMILES: Cc1c(N2CCc3ncc(C(F)(F)F)cc3C2)nn2c(=O)ccnc2c1C1CC1] | 402.2 |
| 1-114 | 9-cyclopropyl-8-methyl-7-(3-(6- methylpyridin-3-yl)-7,8-dihydro- 1,6-naphthyridin-6(5H)-yl)-4H- pyrimido[1,2-b]pyridazin-4-one | 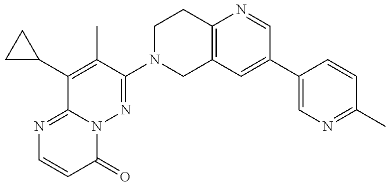 [SMILES: Cc1ccc(-c2cnc3c(c2)CN(c2nn4c(=O)ccnc4c(C4CC4)c2C)CC3)cn1] | 425.2 |
| 1-115 | 2-(difluoromethyl)-8,9-dimethyl-7- (3-(2-methylpyridin-3-yl)-7,8- dihydro-1,6-naphthyridin-6(5H)- yl)-4H-pyrimido[1,2-b]pyridazin-4- one | 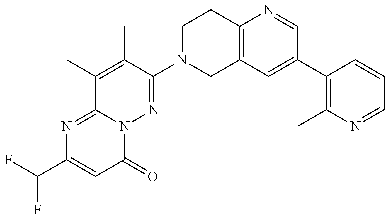 [SMILES: Cc1ncccc1-c1cnc2c(c1)CN(c1nn3c(=O)cc(C(F)F)nc3c(C)c1C)CC2] | 449.3 |
| 1-116 | 2-(difluoromethyl)-8,9-dimethyl-7- (3-(pyrazin-2-yl)-7,8-dihydro-1,6- naphthyridin-6(5H)-yl)-4H- pyrimido[1,2-b]pyridazin-4-one | 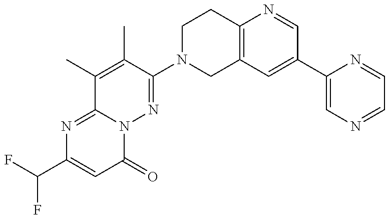 [SMILES: Cc1c(N2CCc3ncc(-c4cnccn4)cc3C2)nn2c(=O)cc(C(F)F)nc2c1C] | 436.2 |
| 1-117 | 2,8,9-trimethyl-7-(3-(4- methylpyridin-3-yl)-7,8-dihydro- 1,6-naphthyridin-6(5H)-yl)-4H- pyrimido[1,2-b]pyridazin-4-one | 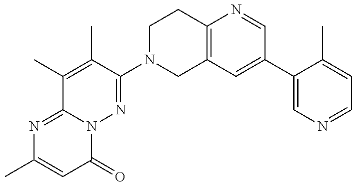 [SMILES: Cc1cc(=O)n2nc(N3CCc4ncc(-c5cnccc5C)cc4C3)c(C)c(C)c2n1] | 413.2 |
| 1-118 | 2,8,9-trimethyl-7-(3-(pyrimidin-5- yl)-7,8-dihydro-1,6-naphthyridin- 6(5H)-yl)-4H-pyrimido[1,2- b]pyridazin-4-one | 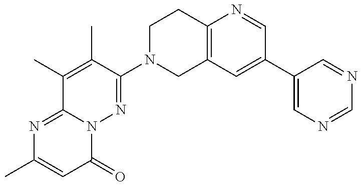 [SMILES: Cc1cc(=O)n2nc(N3CCc4ncc(-c5cncnc5)cc4C3)c(C)c(C)c2n1] | 400.2 |
| 1-119 | 2-(difluoromethyl)-8,9-dimethyl-7- (3-(5-methylpyridin-3-yl)-7,8- dihydro-1,6-naphthyridin-6(5H)- yl)-4H-pyrimido[1,2-b]pyridazin-4- one | 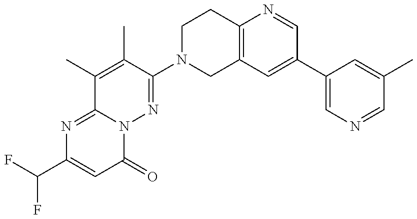 [SMILES: Cc1cncc(-c2cnc3c(c2)CN(c2nn4c(=O)cc(C(F)F)nc4c(C)c2C)CC3)c1] | 449.3 |
| 1-120 | 2,8,9-trimethyl-7-(3-(quinolin-3- yl)-7,8-dihydro-1,6-naphthyridin- 6(5H)-yl)-4H-pyrimido[1,2- b]pyridazin-4-one | 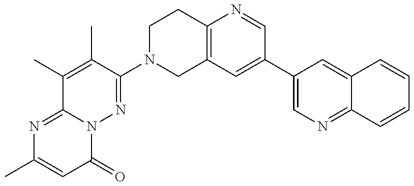 [SMILES: Cc1cc(=O)n2nc(N3CCc4ncc(-c5cnc6ccccc6c5)cc4C3)c(C)c(C)c2n1] | 449.2 |
| 1-121 | 7-(3-(6-(dimethylamino)pyridin-3- yl)-7,8-dihydro-1,6-naphthyridin- 6(5H)-yl)-2,8,9-trimethyl-4H- pyrimido[1,2-b]pyridazin-4-one | 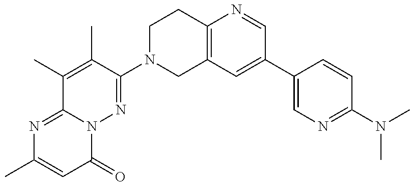 | 442.2 |
| 1-122 | 2,8,9-trimethyl-7-(3-(2- methylpyridin-3-yl)-7,8-dihydro- 1,6-naphthyridin-6(5H)-yl)-4H- pyrimido[1,2-b]pyridazin-4-one | 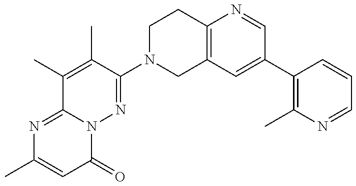 [SMILES: Cc1cc(=O)n2nc(N3CCc4ncc(-c5cccnc5C)cc4C3)c(C)c(C)c2n1] | 413.2 |
| 1-123 | 7-(3-(5-chloropyridin-3-yl)-7,8- dihydro-1,6-naphthyridin-6(5H)- yl)-2,8,9-trimethyl-4H- pyrimido[1,2-b]pyridazin-4-one | 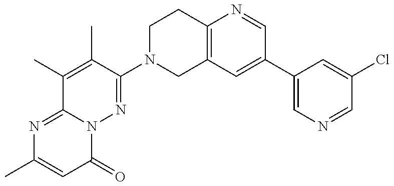 [SMILES: Cc1cc(=O)n2nc(N3CCc4ncc(-c5cncc(Cl)c5)cc4C3)c(C)c(C)c2n1] | 433 |
| 1-124 | 2,8,9-trimethyl-7-(3-(5- (trifluoromethyl)pyridin-3-yl)-7,8- dihydro-1,6-naphthyridin-6(5H)- yl)-4H-pyrimido[1,2-b]pyridazin-4- one | 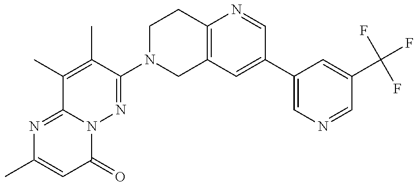 [SMILES: Cc1cc(=O)n2nc(N3CCc4ncc(-c5cncc(C(F)(F)F)c5)cc4C3)c(C)c(C)c2n1] | 467.2 |
| 1-125 | 2,8,9-trimethyl-7-(3-(pyrazin-2-yl)- 7,8-dihydro-1,6-naphthyridin- 6(5H)-yl)-4H-pyrimido[1,2- b]pyridazin-4-one | 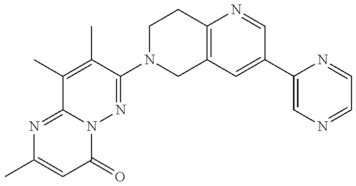 [SMILES: Cc1cc(=O)n2nc(N3CCc4ncc(-c5cnccn5)cc4C3)c(C)c(C)c2n1] | 400 |
| 1-126 | 7-(3-(4-chloropyridin-3-yl)-7,8- dihydro-1,6-naphthyridin-6(5H)- yl)-2,8,9-trimethyl-4H- pyrimido[1,2-b]pyridazin-4-one | 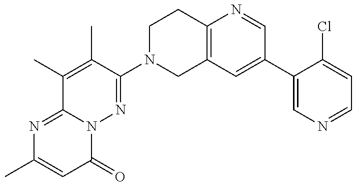 [SMILES: Cc1cc(=O)n2nc(N3CCc4ncc(-c5cnccc5Cl)cc4C3)c(C)c(C)c2n1] | 433.2 |
| 1-127 | 2,8,9-trimethyl-7-(3-(5- methylpyridin-3-yl)-7,8-dihydro- 1,6-naphthyridin-6(5H)-yl)-4H- pyrimido[1,2-b]pyridazin-4-one | 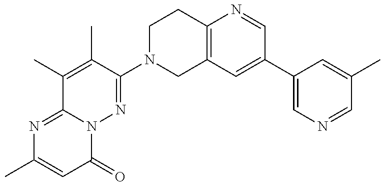 | 413.2 |
| 1-128 | 7-(3-(6-cyclopropylpyridin-3-yl)- 7,8-dihydro-1,6-naphthyridin- 6(5H)-yl)-2,8,9-trimethyl-4H- pyrimido[1,2-b]pyridazin-4-one | 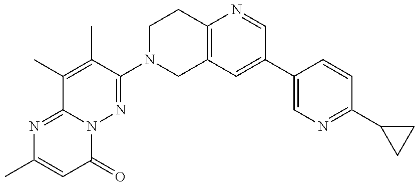 [SMILES: Cc1cc(=O)n2nc(N3CCc4ncc(-c5ccc(C6CC6)nc5)cc4C3)c(C)c(C)c2n1] | 439.2 |
| 1-129 | 2,8,9-trimethyl-7-(3-(pyridin-3-yl)- 7,8-dihydro-1,6-naphthyridin- 6(5H)-yl)-4H-pyrimido[1,2- b]pyridazin-4-one | 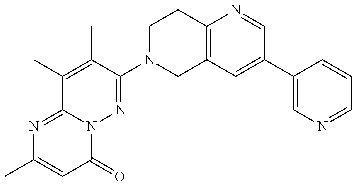 [SMILES: Cc1cc(=O)n2nc(N3CCc4ncc(-c5cccnc5)cc4C3)c(C)c(C)c2n1] | 399.2 |
| 1-130 | 2,8,9-trimethyl-7-(3-(6- (trifluoromethyl)pyridin-3-yl)-7,8- dihydro-1,6-naphthyridin-6(5H)- yl)-4H-pyrimido[1,2-b]pyridazin-4- one | 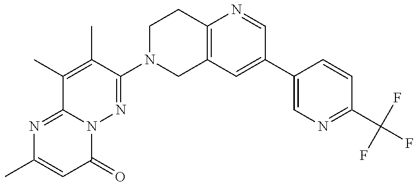 [SMILES: Cc1cc(=O)n2nc(N3CCc4ncc(-c5ccc(C(F)(F)F)nc5)cc4C3)c(C)c(C)c2n1] | 467.2 |
| 1-131 | 2,8,9-trimethyl-7-(3-(7- methylimidazo[1,2-a]pyridin-6-yl)- 7,8-dihydro-1,6-naphthyridin- 6(5H)-yl)-4H-pyrimido[1,2- b]pyridazin-4-one | 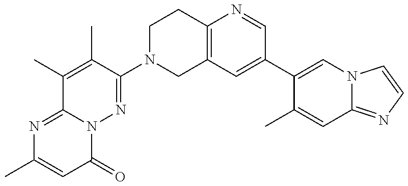 [SMILES: Cc1cc(=O)n2nc(N3CCc4ncc(-c5cn6ccnc6cc5C)cc4C3)c(C)c(C)c2n1] | 452.2 |
| 1-132 | 2,8,9-trimethyl-7-(3-(pyridazin-4- yl)-7,8-dihydro-1,6-naphthyridin- 6(5H)-yl)-4H-pyrimido[1,2- b]pyridazin-4-one | 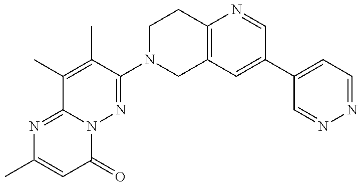 [SMILES: Cc1cc(=O)n2nc(N3CCc4ncc(-c5ccnnc5)cc4C3)c(C)c(C)c2n1] | 400.2 |
| 1-133 | 7-(3-(imidazo[1,2-a]pyridin-6-yl)- 7,8-dihydro-1,6-naphthyridin- 6(5H)-yl)-2,8,9-trimethyl-4H- pyrimido[1,2-b]pyridazin-4-one | 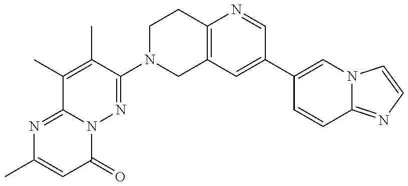 [SMILES: Cc1cc(=O)n2nc(N3CCc4ncc(-c5ccc6nccn6c5)cc4C3)c(C)c(C)c2n1] | 438.2 |
| 1-134 | 7-(3-(5-fluoropyridin-3-yl)-7,8- dihydro-1,6-naphthyridin-6(5H)- yl)-2,8,9-trimethyl-4H- pyrimido[1,2-b]pyridazin-4-one | 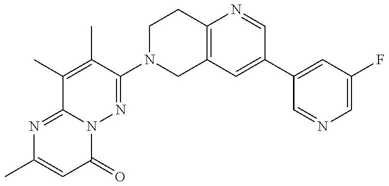 [SMILES: Cc1cc(=O)n2nc(N3CCc4ncc(-c5cncc(F)c5)cc4C3)c(C)c(C)c2n1] | 417.2 |
| 1-135 | 2,8,9-trimethyl-7-(3-(p-tolyl)-7,8- dihydro-1,6-naphthyridin-6(5H)- yl)-4H-pyrimido[1,2-b]pyridazin-4- one | 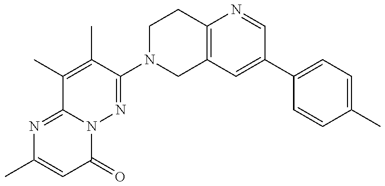 | 412.2 |
| 1-136 | 7-(3-(2,4-dimethylphenyl)-7,8- dihydro-1,6-naphthyridin-6(5H)- yl)-2,8,9-trimethyl-4H- pyrimido[1,2-b]pyridazin-4-one | 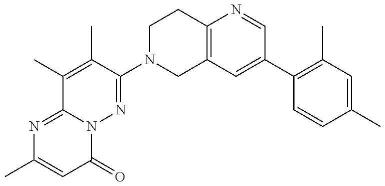 | 426.2 |
| 1-137 | 2,8,9-trimethyl-7-(3-(o-tolyl)-7,8- dihydro-1,6-naphthyridin-6(5H)- yl)-4H-pyrimido[1,2-b]pyridazin-4- one | 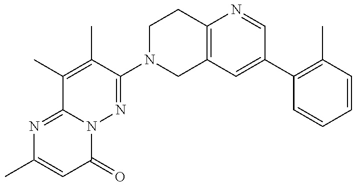 [SMILES: Cc1cc(=O)n2nc(N3CCc4ncc(-c5ccccc5C)cc4C3)c(C)c(C)c2n1] | 412.2 |
| 1-138 | 7-(3-(5-fluoro-2-methylphenyl)-7,8- dihydro-1,6-naphthyridin-6(5H)- yl)-2,8,9-trimethyl-4H- pyrimido[1,2-b]pyridazin-4-one | 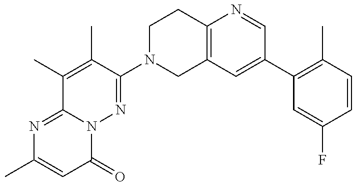 [SMILES: Cc1cc(=O)n2nc(N3CCc4ncc(-c5cc(F)ccc5C)cc4C3)c(C)c(C)c2n1] | 430.2 |
| 1-139 | 7-(3-(6-methoxy-4-methylpyridin- 3-yl)-7,8-dihydro-1,6-naphthyridin- 6(5H)-yl)-8,9-dimethyl-4H- pyrimido[1,2-b]pyridazin-4-one | 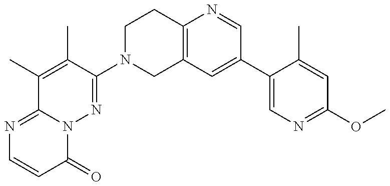 [SMILES: COc1cc(C)c(-c2cnc3c(c2)CN(c2nn4c(=O)ccnc4c(C)c2C)CC3)cn1] | 429.4 |
| 1-140 | 7-(3-(4-methoxypyridin-3-yl)-7,8- dihydro-1,6-naphthyridin-6(5H)- yl)-8,9-dimethyl-4H-pyrimido[1,2- b]pyridazin-4-one | 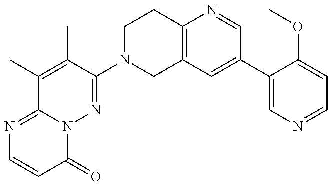 [SMILES: COc1ccncc1-c1cnc2c(c1)CN(c1nn3c(=O)ccnc3c(C)c1C)CC2] | 415.4 |
| 1-141 | 8,9-dimethyl-7-(3-(pyridazin-4-yl)- 7,8-dihydro-1,6-naphthyridin- 6(5H)-yl)-4H-pyrimido[1,2- b]pyridazin-4-one | 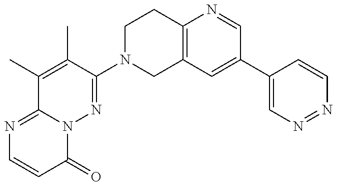 [SMILES: Cc1c(N2CCc3ncc(-c4ccnnc4)cc3C2)nn2c(=O)ccnc2c1C] | 386.4 |
| 1-142 | 8,9-dimethyl-7-(3-(4- (trifluoromethyl)pyridin-3-yl)-7,8- dihydro-naphthyridin-6(5H)- yl)-4H-pyrimido[1,2-b]pyridazin-4- one | 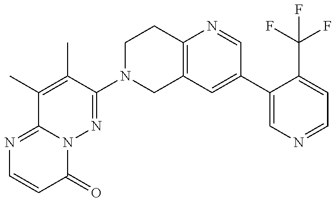 [SMILES: Cc1c(N2CCc3ncc(-c4cnccc4C(F)(F)F)cc3C2)nn2c(=O)ccnc2c1C] | 453.3 |
| 1-143 | 7-(3-(3-fluoro-4-methylphenyl)-7,8- dihydro-1,6-naphthyridin-6(5H)- yl)-8,9-dimethyl-4H-pyrimido[1,2- b]pyridazin-4-one | 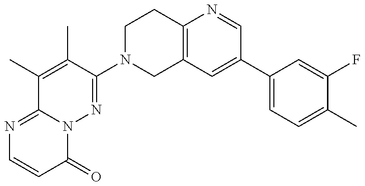 [SMILES: Cc1ccc(-c2cnc3c(c2)CN(c2nn4c(=O)ccnc4c(C)c2C)CC3)cc1F] | 416.5 |
| 1-144 | 7-(3-(5-chloropyridin-3-yl)-7,8- dihydro-1,6-naphthyridin-6(5H)- yl)-8,9-dimethyl-4H-pyrimido[1,2- b]pyridazin-4-one | 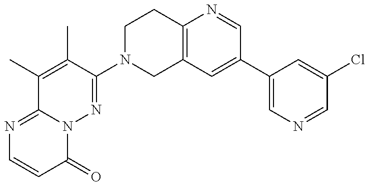 [SMILES: Cc1c(N2CCc3ncc(-c4cncc(Cl)c4)cc3C2)nn2c(=O)ccnc2c1C] | 419.3 |
| 1-145 | 7-(3-(4-chloropyridin-3-yl)-7,8- dihydro-1,6-naphthyridin-6(5H)- yl)-8,9-dimethyl-4H-pyrimido[1,2- b]pyridazin-4-one | 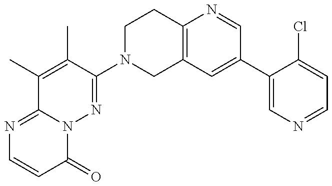 [SMILES: Cc1c(N2CCc3ncc(-c4cnccc4Cl)cc3C2)nn2c(=O)ccnc2c1C] | 419.2 |
| 1-146 | 7-(3-(6-chloro-5-fluoropyridin-3- yl)-7,8-dihydro-1,6-naphthyridin- 6(5H)-yl)-8,9-dimethyl-4H- pyrimido[1,2-b]pyridazin-4-one | 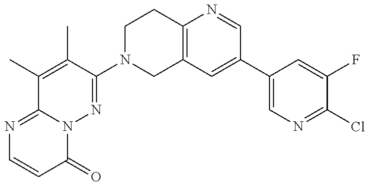 [SMILES: Cc1c(N2CCc3ncc(-c4cnc(Cl)c(F)c4)cc3C2)nn2c(=O)ccnc2c1C] | 437.2 |
| 1-147 | 8,9-dimethyl-7-(3-(5- methylpyridin-3-yl)-7,8-dihydro- 1,6-naphthyridin-6(5H)-yl)-4H- pyrimido[1,2-b]pyridazin-4-one | 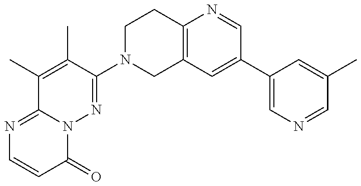 [SMILES: Cc1cncc(-c2cnc3c(c2)CN(c2nn4c(=O)ccnc4c(C)c2C)CC3)c1] | 399.4 |
| 1-148 | 8,9-dimethyl-7-(3-(pyridin-3-yl)- 7,8-dihydro-1,6-naphthyridin- 6(5H)-yl)-4H-pyrimido[1,2- b]pyridazin-4-one | 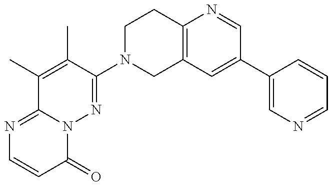 [SMILES: Cc1c(N2CCc3ncc(-c4cccnc4)cc3C2)nn2c(=O)ccnc2c1C] | 385.3 |
| 1-149 | 7-(3-(6-fluoro-4-methylpyridin-3- yl)-7,8-dihydro-1,6-naphthyridin- 6(5H)-yl)-8,9-dimethyl-4H- pyrimido[1,2-b]pyridazin-4-one | 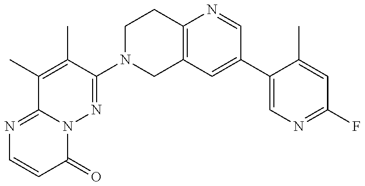 [SMILES: Cc1cc(F)ncc1-c1cnc2c(c1)CN(c1nn3c(=O)ccnc3c(C)c1C)CC2] | 417.4 |
| 1-150 | 8,9-dimethyl-7-(3-(1-(tetrahydro- 2H-pyran-2-yl)-1H-pyrazol-4-yl)- 7,8-dihydro-1,6-naphthyridin- 6(5H)-yl)-4H-pyrimido[1,2- b]pyridazin-4-one | 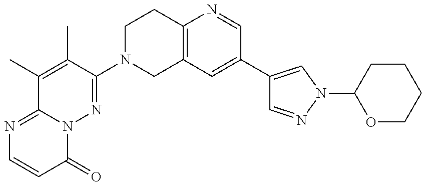 [SMILES: Cc1c(N2CCc3ncc(-c4cnn(C5CCCCO5)c4)cc3C2)nn2c(=O)ccnc2c1C] | 458.4 |
| 1-151 | 8,9-dimethyl-7-(3-(1-(oxetan-3-yl)- 1H-pyrazol-5-yl)-7,8-dihydro-1,6- naphthyridin-6(5H)-yl)-4H- pyrimido[1,2-b]pyridazin-4-one | 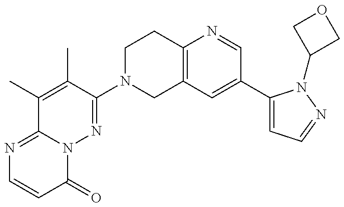 [SMILES: Cc1c(N2CCc3ncc(-c4ccnn4C4COC4)cc3C2)nn2c(=O)ccnc2c1C] | 430.4 |
| 1-152 | 7-(3-(1-cyclopropyl-1H-pyrazol-4- yl)-7,8-dihydro-1,6-naphthyridin- 6(5H)-yl)-8,9-dimethyl-4H- pyrimido[1,2-b]pyridazin-4-one | 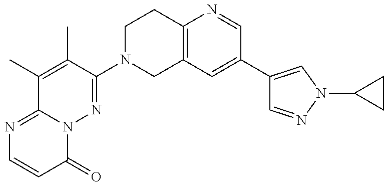 [SMILES: Cc1c(N2CCc3ncc(-c4cnn(C5CC5)c4)cc3C2)nn2c(=O)ccnc2c1C] | 414.4 |
| 1-153 | 8,9-dimethyl-7-(3-(1-(pyridin-2-yl)- 1H-pyrazol-4-yl)-7,8-dihydro-1,6- naphthyridin-6(5H)-yl)-4H- pyrimido[1,2-b]pyridazin-4-one | 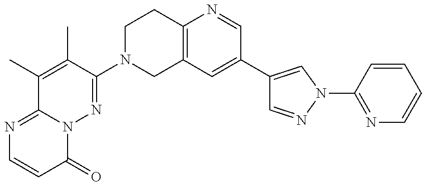 [SMILES: Cc1c(N2CCc3ncc(-c4cnn(-c5ccccn5)c4)cc3C2)nn2c(=O)ccnc2c1C] | 451.4 |
| 1-154 | 2-(difluoromethyl)-8,9-dimethyl-7- (3-(5-(trifluoromethyl)pyridin-3- yl)-7,8-dihydro-1,6-naphthyridin- 6(5H)-yl)-4H-pyrimido[1,2- b]pyridazin-4-one | 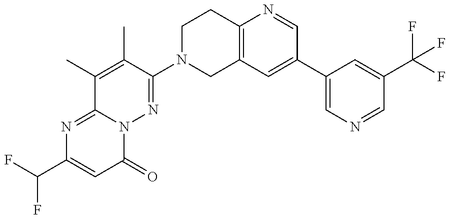 [SMILES: Cc1c(N2CCc3ncc(-c4cncc(C(F)(F)F)c4)cc3C2)nn2c(=O)cc(C(F)F)nc2c1C] | 478.2 |
| 1-155 | 2-(difluoromethyl)-8,9-dimethyl-7- (3-(pyridin-3-yl)-7,8-dihydro-1,6- naphthyridin-6(5H)-yl)-4H- pyrimido[1,2-b]pyridazin-4-one | 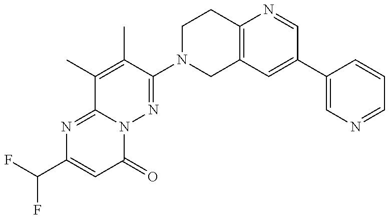 [SMILES: Cc1c(N2CCc3ncc(-c4cccnc4)cc3C2)nn2c(=O)cc(C(F)F)nc2c1C] | 503 |
| 1-156 | 2-(difluoromethyl)-7-(3-(5- fluoropyridin-3-yl)-7,8-dihydro-1,6- naphthyridin-6(5H)-yl)-8,9- dimethyl-4H-pyrimido[1,2- b]pyridazin-4-one | 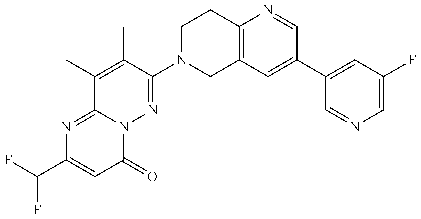 [SMILES: Cc1c(N2CCc3ncc(-c4cncc(F)c4)cc3C2)nn2c(=O)cc(C(F)F)nc2c1C] | 475.2 |
| 1-157 | 5-(6-(2-(difluoromethyl)-8,9- dimethyl-4-oxo-4H-pyrimido[1,2- b]pyridazin-7-yl)-5,6,7,8- tetrahydro-1,6-naphthyridin-3- yl)nicotinonitrile | 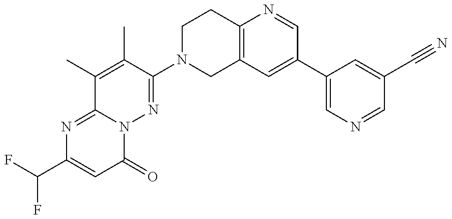 [SMILES: Cc1c(N2CCc3ncc(-c4cncc(C#N)c4)cc3C2)nn2c(=O)cc(C(F)F)nc2c1C] | 460 |
| 1-158 | 2-(difluoromethyl)-8,9-dimethyl-7- (3-(pyridazin-4-yl)-7,8-dihydro-1,6- naphthyridin-6(5H)-yl)-4H- pyrimido[1,2-b]pyridazin-4-one | 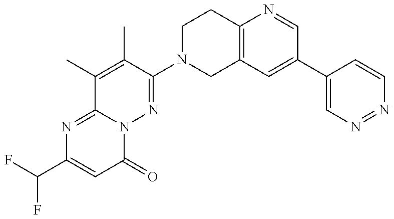 [SMILES: Cc1c(N2CCc3ncc(-c4ccnnc4)cc3C2)nn2c(=O)cc(C(F)F)nc2c1C] | 436.2 |
| 1-159 | 2-(difluoromethyl)-8,9-dimethyl-7- (3-(pyrimidin-5-yl)-7,8-dihydro- 1,6-naphthyridin-6(5H)-yl)-4H- pyrimido[1,2-b]pyridazin-4-one | 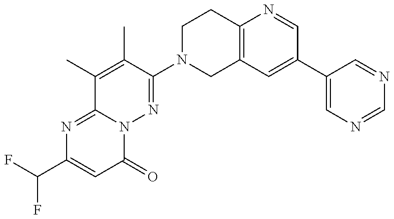 [SMILES: Cc1c(N2CCc3ncc(-c4cncnc4)cc3C2)nn2c(=O)cc(C(F)F)nc2c1C] | 436 |
| 1-160 | 7-(3-(5-chloropyridin-3-yl)-7,8- dihydro-1,6-naphthyridin-6(5H)- yl)-2-(difluoromethyl)-8,9- dimethyl-4H-pyrimido[1,2- b]pyridazin-4-one | 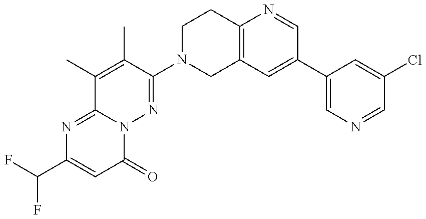 [SMILES: Cc1c(N2CCc3ncc(-c4cncc(Cl)c4)cc3C2)nn2c(=O)cc(C(F)F)nc2c1C] | 469 |
| 1-161 | 2-(difluoromethyl)-8,9-dimethyl-7- (3-(4-methylpyridin-3-yl)-7,8- dihydro-1,6-naphthyridin-6(5H)- yl)-4H-pyrimido[1,2-b]pyridazin-4- one | 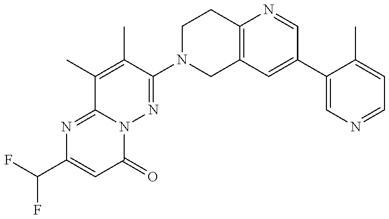 [SMILES: Cc1ccncc1-c1cnc2c(c1)CN(c1nn3c(=O)cc(C(F)F)nc3c(C)c1C)CC2] | 449.2 |
| 1-162 | 2-(difluoromethyl)-8,9-dimethyl-7- (3-(7-methylimidazo[1,2-a]pyridin- 6-yl)-7,8-dihydro-1,6-naphthyridin- 6(5H)-yl)-4H-pyrimido[1,2- b]pyridazin-4-one | 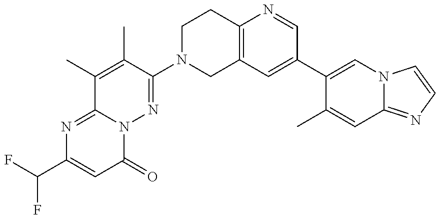 [SMILES: Cc1cc2nccn2cc1-c1cnc2c(c1)CN(c1nn3c(=O)cc(C(F)F)nc3c(C)c1C)CC2] | 488.2 |
| 1-163 | 2-(difluoromethyl)-8,9-dimethyl-7- (3-(1-(pyridin-2-yl)-1H-pyrazol-4- yl)-7,8-dihydro-1,6-naphthyridin- 6(5H)-yl)-4H-pyrimido[1,2- b]pyridazin-4-one | 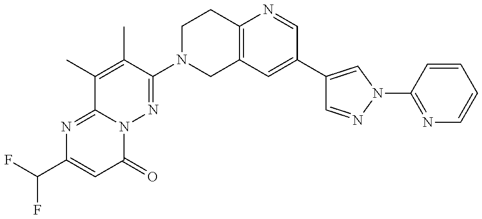 [SMILES: Cc1c(N2CCc3ncc(-c4cnn(-c5ccccn5)c4)cc3C2)nn2c(=O)cc(C(F)F)nc2c1C] | 501.2 |
| 1-164 | 2-(difluoromethyl)-7-(3-(2,4- dimethylphenyl)-7,8-dihydro-1,6- naphthyridin-6(5H)-yl)-8,9- dimethyl-4H-pyrimido[1,2- b]pyridazin-4-one | 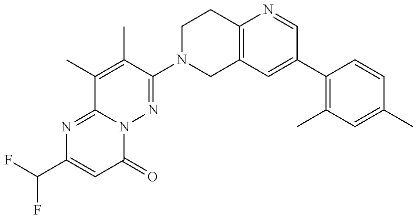 [SMILES: Cc1ccc(-c2cnc3c(c2)CN(c2nn4c(=O)cc(C(F)F)nc4c(C)c2C)CC3)c(C)c1] | 462.2 |
| 1-165 | 2-(difluoromethyl)-7-(3-(1-(2- fluorophenyl)-1H-pyrazol-4-yl)- 7,8-dihydro-1,6-naphthyridin- 6(5H)-yl)-8,9-dimethyl-4H- pyrimido[1,2-b]pyridazin-4-one | 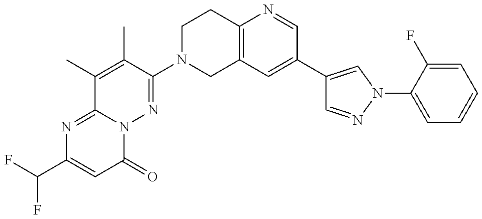 [SMILES: Cc1c(N2CCc3ncc(-c4cnn(-c5ccccc5F)c4)cc3C2)nn2c(=O)cc(C(F)F)nc2c1C] | 518.2 |
| 1-166 | 2-(difluoromethyl)-7-(3-(1-ethyl- 1H-pyrazol-4-yl)-7,8-dihydro-1,6- naphthyridin-6(5H)-yl)-8,9- dimethyl-4H-pyrimido[1,2- b]pyridazin-4-one | 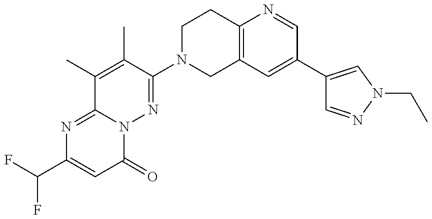 [SMILES: CCn1cc(-c2cnc3c(c2)CN(c2nn4c(=O)cc(C(F)F)nc4c(C)c2C)CC3)cn1] | 452.2 |
| 1-167 | 2-(difluoromethyl)-7-(3-(5- methoxypyridin-3-yl)-7,8-dihydro- 1,6-naphthyridin-6(5H)-yl)-8,9- dimethyl-4H-pyrimido[1,2- b]pyridazin-4-one | 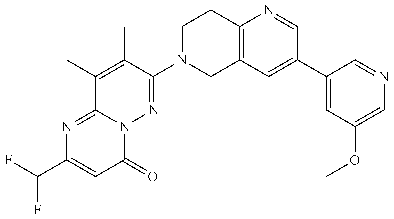 [SMILES: COc1cncc(-c2cnc3c(c2)CN(c2nn4c(=O)cc(C(F)F)nc4c(C)c2C)CC3)c1] | 465.2 |
| 1-168 | 7-(3-(5-chloro-6-methoxypyridin-3- yl)-7,8-dihydro-1,6-naphthyridin- 6(5H)-yl)-2-(difluoromethyl)-8,9- dimethyl-4H-pyrimido[1,2- b]pyridazin-4-one | 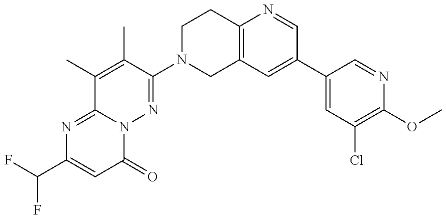 [SMILES: COc1ncc(-c2cnc3c(c2)CN(c2nn4c(=O)cc(C(F)F)nc4c(C)c2C)CC3)cc1Cl] | 499 |
| 1-169 | 2-(difluoromethyl)-8,9-dimethyl-7- (3-(p-tolyl)-7,8-dihydro-1,6- naphthyridin-6(5H)-yl)-4H- pyrimido[1,2-b]pyridazin-4-one | 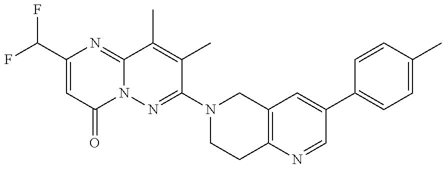 [SMILES: Cc1ccc(-c2cnc3c(c2)CN(c2nn4c(=O)cc(C(F)F)nc4c(C)c2C)CC3)cc1] | 448.2 |
| 1-170 | 2-(difluoromethyl)-7-(3-(6- methoxypyridin-3-yl)-7,8-dihydro- 1,6-naphthyridin-6(5H)-yl)-8,9- dimethyl-4H-pyrimido[1,2- b]pyridazin-4-one | 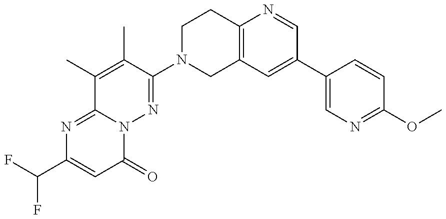 [SMILES: COc1ccc(-c2cnc3c(c2)CN(c2nn4c(=O)cc(C(F)F)nc4c(C)c2C)CC3)cn1] | 465.2 |
| 1-171 | 2-(difluoromethyl)-7-(3-(5-fluoro- 2-methylphenyl)-7,8-dihydro-1,6- naphthyridin-6(5H)-yl)-8,9- dimethyl-4H-pyrimido[1,2- b]pyridazin-4-one | 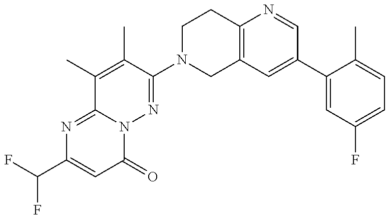 [SMILES: Cc1ccc(F)cc1-c1cnc2c(c1)CN(c1nn3c(=O)cc(C(F)F)nc3c(C)c1C)CC2] | 466.2 |
| 1-172 | 2-(difluoromethyl)-7-(3-(1- (difluoromethyl)-1H-pyrazol-4-yl)- 7,8-dihydro-1,6-naphthyridin- 6(5H)-yl)-8,9-dimethyl-4H- pyrimido[1,2-b]pyridazin-4-one | 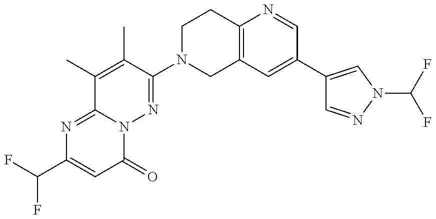 [SMILES: Cc1c(N2CCc3ncc(-c4cnn(C(F)F)c4)cc3C2)nn2c(=O)cc(C(F)F)nc2c1C] | 474.2 |
| 1-173 | 2-(difluoromethyl)-8,9-dimethyl-7- (3-(1-(2-morpholinoethyl)-1H- pyrazol-4-yl)-7,8-dihydro-1,6- naphthyridin-6(5H)-yl)-4H- pyrimido[1,2-b]pyridazin-4-one | 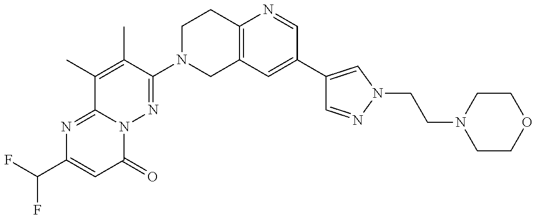 [SMILES: Cc1c(N2CCc3ncc(-c4cnn(CCN5CCOCC5)c4)cc3C2)nn2c(=O)cc(C(F)F)nc2c1C] | 537.2 |
| 1-174 | 7-(3-(2,6-dichloropyridin-3-yl)-7,8- dihydro-1,6-naphthyridin-6(5H)- yl)-2-(difluoromethyl)-8,9- dimethyl-4H-pyrimido[1,2- b]pyridazin-4-one | 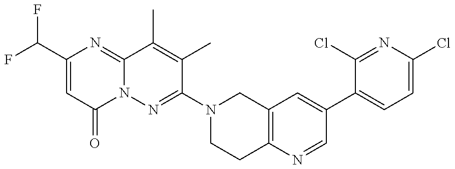 [SMILES: Cc1c(N2CCc3ncc(-c4ccc(Cl)nc4Cl)cc3C2)nn2c(=O)cc(C(F)F)nc2c1C] | 503 |
| 1-175 | 2,8,9-trimethyl-7-(3-(1-(pyridin-2- yl)-1H-pyrazol-4-yl)-7,8-dihydro- 1,6-naphthyridin-6(5H)-yl)-4H- pyrimido[1,2-b]pyridazin-4-one | 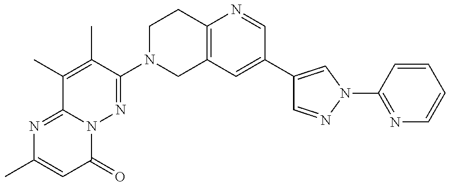 [SMILES: Cc1cc(=O)n2nc(N3CCc4ncc(-c5cnn(-c6ccccn6)c5)cc4C3)c(C)c(C)c2n1] | 465.4 |
| 1-176 | 2,8,9-trimethyl-7-(3-(1-(oxetan-3- yl)-1H-pyrazol-5-yl)-7,8-dihydro- 1,6-naphthyridin-6(5H)-yl)-4H- pyrimido[1,2-b]pyridazin-4-one | 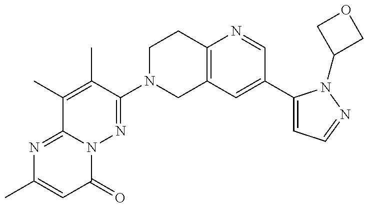 [SMILES: Cc1cc(=O)n2nc(N3CCc4ncc(-c5ccnn5C5COC5)cc4C3)c(C)c(C)c2n1] | 444.4 |
| 1-177 | 7-(3-(1-(2-(dimethylamino)ethyl)- 1H-pyrazol-4-yl)-7,8-dihydro-1,6- naphthyridin-6(5H)-yl)-2,8,9- trimethyl-4H-pyrimido[1,2- b]pyridazin-4-one | 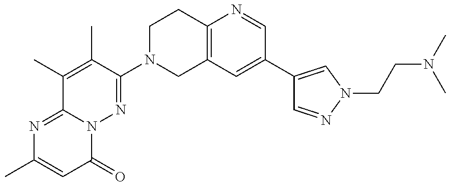 | 459.4 |
| 1-178 | 2,8,9-trimethyl-7-(3-(1-(2- morpholinoethyl)-1H-pyrazol-4-yl)- 7,8-dihydro-1,6-naphthyridin- 6(5H)-yl)-4H-pyrimido[1,2- b]pyridazin-4-one | 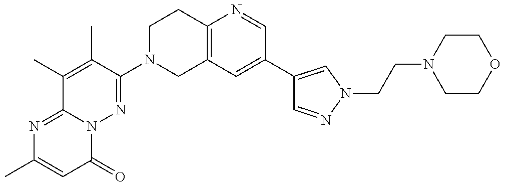 [SMILES: Cc1cc(=O)n2nc(N3CCc4ncc(-c5cnn(CCN6CCOCC6)c5)cc4C3)c(C)c(C)c2n1] | 501.4 |
| 1-179 | 2,8,9-trimethyl-7-(3-(1-(oxetan-3- yl)-1H-pyrazol-4-yl)-7,8-dihydro- 1,6-naphthyridin-6(5H)-yl)-4H- pyrimido[1,2-b]pyridazin-4-one | 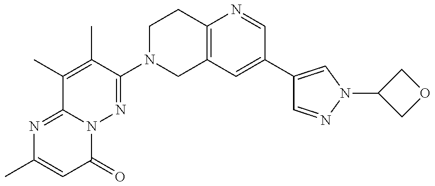 [SMILES: Cc1cc(=O)n2nc(N3CCc4ncc(-c5cnn(C6COC6)c5)cc4C3)c(C)c(C)c2n1] | 444.4 |
| 1-180 | 7-(3-(1-(2-fluorophenyl)-1H- pyrazol-4-yl)-7,8-dihydro-1,6- naphthyridin-6(5H)-yl)-2,8,9- trimethyl-4H-pyrimido[1,2- b]pyridazin-4-one | 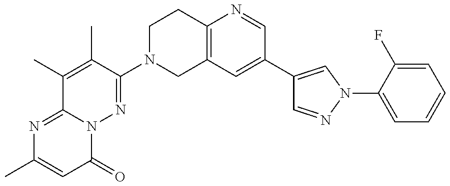 [SMILES: Cc1cc(=O)n2nc(N3CCc4ncc(-c5cnn(-c6ccccc6F)c5)cc4C3)c(C)c(C)c2n1] | 482.4 |
| 1-181 | 2,8,9-trimethyl-7-(3-(pyrazolo[1,5- a]pyridin-3-yl)-7,8-dihydro-1,6- naphthyridin-6(5H)-yl)-4H- pyrimido[1,2-b]pyridazin-4-one | 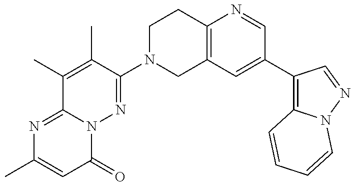 [SMILES: Cc1cc(=O)n2nc(N3CCc4ncc(-c5cnn6ccccc56)cc4C3)c(C)c(C)c2n1] | 438.4 |
| 1-182 | 2,8,9-trimethyl-7-(3-(1-(tetrahydro- 2H-pyran-4-yl)-1H-pyrazol-4-yl)- 7,8-dihydro-1,6-naphthyridin- 6(5H)-yl)-4H-pyrimido[1,2- b]pyridazin-4-one | 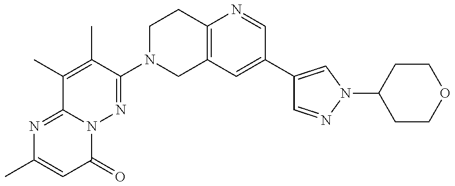 [SMILES: Cc1cc(=O)n2nc(N3CCc4ncc(-c5cnn(C6CCOCC6)c5)cc4C3)c(C)c(C)c2n1] | 472.4 |
| 1-183 | 7-(3-(1-cyclopropyl-1H-pyrazol-4- yl)-7,8-dihydro-1,6-naphthyridin- 6(5H)-yl)-2,8,9-trimethyl-4H- pyrimido[1,2-b]pyridazin-4-one | 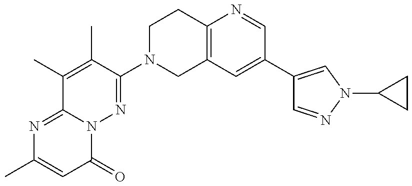 [SMILES: Cc1cc(=O)n2nc(N3CCc4ncc(-c5cnn(C6CC6)c5)cc4C3)c(C)c(C)c2n1] | 428.4 |
| 1-184 | 2,8,9-trimethyl-7-(3-(1-(2,2,2- trifluoroethyl)-1H-pyrazol-4-yl)- 7,8-dihydro-1,6-naphthyridin- 6(5H)-yl)-4H-pyrimido[1,2- b]pyridazin-4-one | 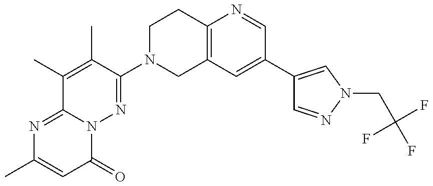 [SMILES: Cc1cc(=O)n2nc(N3CCc4ncc(-c5cnn(CC(F)(F)F)c5)cc4C3)c(C)c(C)c2n1] | 470.4 |
| 1-185 | 7-(3-(1,5-dimethyl-1H-pyrazol-4- yl)-7,8-dihydro-1,6-naphthyridin- 6(5H)-yl)-2,8,9-trimethyl-4H- pyrimido[1,2-b]pyridazin-4-one | 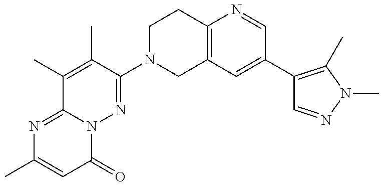 | 416.4 |
| 1-186 | 7-(3-(6-methoxypyridin-3-yl)-7,8- dihydro-1,6-naphthyridin-6(5H)- yl)-2,8,9-trimethyl-4H- pyrimido[1,2-b]pyridazin-4-one | 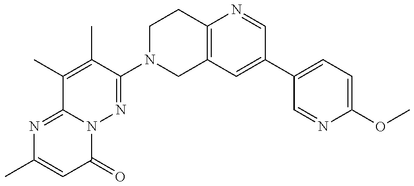 | 429.4 |
| 1-187 | 7-(3-(4-chloro-6-methylpyridin-3- yl)-7,8-dihydro-1,6-naphthyridin- 6(5H)-yl)-2-(difluoromethyl)-8,9- dimethyl-4H-pyrimido[1,2- b]pyridazin-4-one | 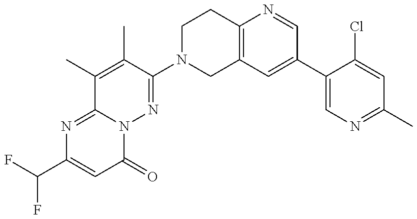 [SMILES: Cc1cc(Cl)c(-c2cnc3c(c2)CN(c2nn4c(=O)cc(C(F)F)nc4c(C)c2C)CC3)cn1] | 483.2 |
| 1-188 | 2-(difluoromethyl)-7-(3-(6- methoxy-4-methylpyridin-3-yl)-7,8- dihydro-1,6-naphthyridin- 6(5H)-yl)-8,9-dimethyl-4H- pyrimido[1,2-b]pyridazin-4-one | 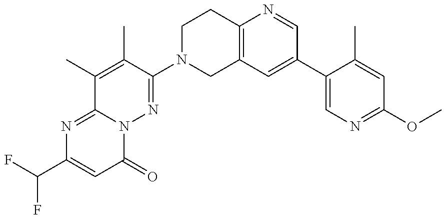 [SMILES: COc1cc(C)c(-c2cnc3c(c2)CN(c2nn4c(=O)cc(C(F)F)nc4c(C)c2C)CC3)cn1] | 479.4 |
| 1-189 | 2-(difluoromethyl)-8,9-dimethyl-7- (3-(4-(trifluoromethyl)pyridin-3- yl)-7,8-dihydro-1,6-naphthyridin- 6(5H)-yl)-4H-pyrimido[1,2- b]pyridazin-4-one | 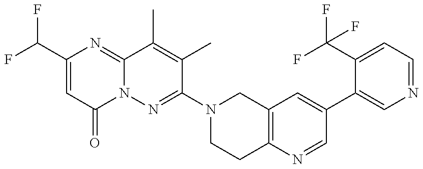 [SMILES: Cc1c(N2CCc3ncc(-c4cnccc4C(F)(F)F)cc3C2)nn2c(=O)cc(C(F)F)nc2c1C] | 503.3 |
| 1-190 | 2-(difluoromethyl)-7-(3-(4- methoxypyridin-3-yl)-7,8-dihydro- 1,6-naphthyridin- 6(5H)-yl)-8,9-dimethyl-4H- pyrimido[1,2-b]pyridazin-4-one | 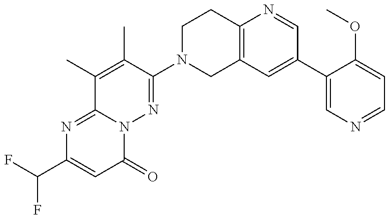 [SMILES: COc1ccncc1-c1cnc2c(c1)CN(c1nn3c(=O)cc(C(F)F)nc3c(C)c1C)CC2] | 465.3 |
| 1-191 | 2-(difluoromethyl)-7-(3-(3-fluoro- 4-methylphenyl)-7,8-dihydro- 1,6-naphthyridin- 6(5H)-yl)-8,9-dimethyl-4H- pyrimido[1,2-b]pyridazin-4-one | 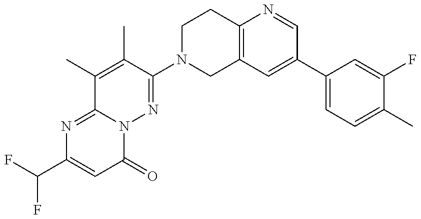 [SMILES: Cc1ccc(-c2cnc3c(c2)CN(c2nn4c(=O)cc(C(F)F)nc4c(C)c2C)CC3)cc1F] | 466.3 |
| 1-192 | 8,9-dimethyl-7-(3-(1-(tetrahydro- 2H-pyran-4-yl)-1H-pyrazol-4-yl)- 7,8-dihydro-1,6-naphthyridin- 6(5H)-yl)-4H-pyrimido[1,2- b]pyridazin-4-one | 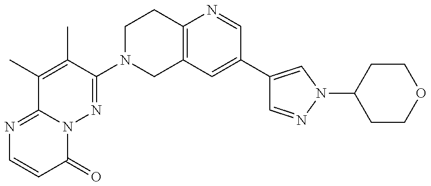 [SMILES: Cc1c(N2CCc3ncc(-c4cnn(C5CCOCC5)c4)cc3C2)nn2c(=O)ccnc2c1C] | 458.4 |
| 1-193 | 7-(3-(1-(2-fluorophenyl)-1H- pyrazol-4-yl)-7,8-dihydro- 1,6-naphthyridin- 6(5H)-yl)-8,9-dimethyl-4H- pyrimido[1,2-b]pyridazin-4-one | 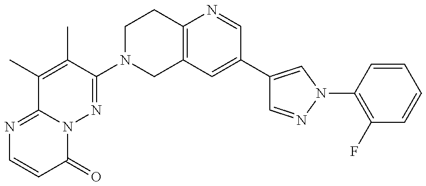 [SMILES: Cc1c(N2CCc3ncc(-c4cnn(-c5ccccc5F)c4)cc3C2)nn2c(=O)ccnc2c1C] | 468.4 |
| 1-194 | 7-(3-(5-fluoro-2-methylphenyl)-7,8- dihydro-1,6-naphthyridin- 6(5H)-yl)-8,9-dimethyl-4H- pyrimido[1,2-b]pyridazin-4-one | 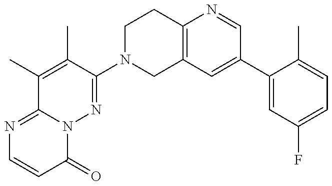 [SMILES: Cc1ccc(F)cc1-c1cnc2c(c1)CN(c1nn3c(=O)ccnc3c(C)c1C)CC2] | 416.4 |
| 1-195 | 2-(2-hydroxypropan-2-yl)-8- methyl-7-(3-(trifluoromethyl)-7,8- dihydro-1,6-naphthyridin- 6(5H)-yl)-4H-pyrimido[1,2-b] pyridazin-4-one | 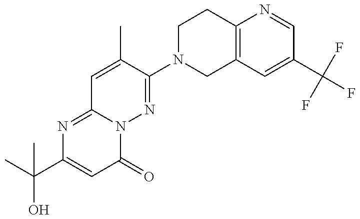 [SMILES: Cc1cc2nc(C(C)(C)O)cc(=O)n2nc1N1CCc2ncc(C(F)(F)F)cc2C1] | 420 |
| 1-196 | 7-(3-(3-fluoro-4-methylphenyl)-7,8- dihydro-1,6-naphthyridin- 6(5H)-yl)-2,8,9-trimethyl-4H- pyrimido[1,2-b]pyridazin-4-one | 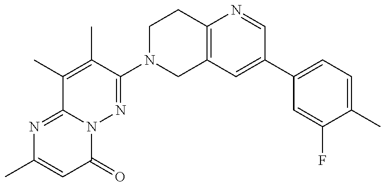 | 430.2 |
| 1-197 | 7-(3-(6-chloro-4-methylpyridin-3- yl)-7,8-dihydro-1,6-naphthyridin- 6(5H)-yl)-2,8,9-trimethyl-4H- pyrimido[1,2-b]pyridazin-4-one | 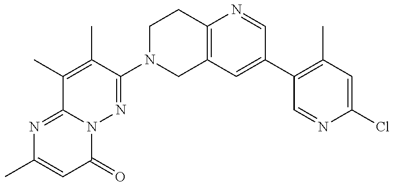 [SMILES: Cc1cc(=O)n2nc(N3CCc4ncc(-c5cnc(Cl)cc5C)cc4C3)c(C)c(C)c2n1] | 447.2 |
| 1-198 | 7-(3-(6-methoxy-4-methylpyridin- 3-yl)-7,8-dihydro-1,6-naphthyridin- 6(5H)-yl)-2,8,9-trimethyl-4H- pyrimido[1,2-b]pyridazin-4-one | 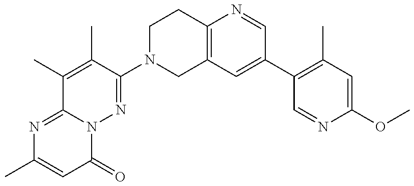 | 443.2 |
| 1-199 | 7-(3-(5-methoxypyridin-3-yl)-7,8- dihydro-1,6-naphthyridin- 6(5H)-yl)-2,8,9-trimethyl-4H- pyrimido[1,2-b]pyridazin-4-one | 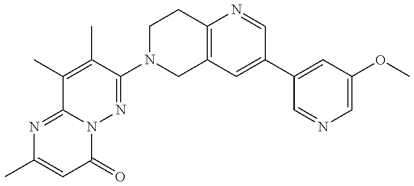 | 429.2 |
| 1-200 | 2,8,9-trimethyl-7-(3-(2,4,5- trifluorophenyl)-7,8-dihydro- 1,6-naphthyridin- 6(5H)-yl)-4H-pyrimido[1,2-b] pyridazin-4-one | 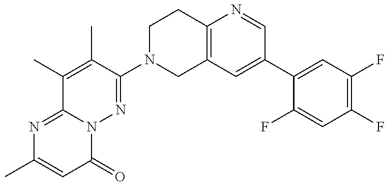 [SMILES: Cc1cc(=O)n2nc(N3CCc4ncc(-c5cc(F)c(F)cc5F)cc4C3)c(C)c(C)c2n1] | 452.2 |
| 1-201 | 7-(3-(3-fluoro-4-methoxyphenyl)- 7,8-dihydro-1,6-naphthyridin- 6(5H)-yl)-2,8,9-trimethyl-4H- pyrimido[1,2-b]pyridazin-4-one | 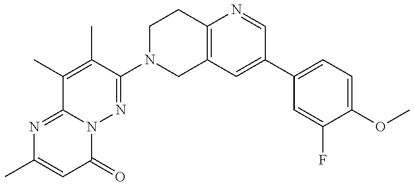 | 446.2 |
| 1-202 | 7-(3-(6-fluoro-4-methylpyridin-3- yl)-7,8-dihydro-1,6-naphthyridin- 6(5H)-yl)-2,8,9-trimethyl-4H- pyrimido[1,2-b]pyridazin-4-one | 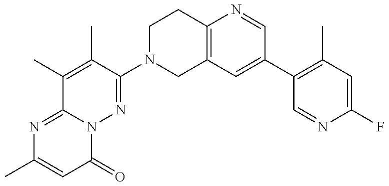 [SMILES: Cc1cc(=O)n2nc(N3CCc4ncc(-c5cnc(F)cc5C)cc4C3)c(C)c(C)c2n1] | 431.2 |
| 1-203 | 7-(3-(3-fluorophenyl)-7,8-dihydro- 1,6-naphthyridin- 6(5H)-yl)-2,8,9-trimethyl-4H- pyrimido[1,2-b]pyridazin-4-one | 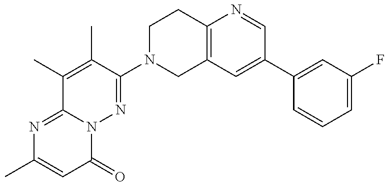 [SMILES: Cc1cc(=O)n2nc(N3CCc4ncc(-c5cccc(F)c5)cc4C3)c(C)c(C)c2n1] | 416.2 |
| 1-204 | 7-(3-(6-fluoropyridin-3-yl)-7,8- dihydro-1,6-naphthyridin- 6(5H)-yl)-2,8,9-trimethyl-4H- pyrimido[1,2-b]pyridazin-4-one | 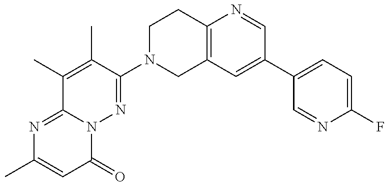 [SMILES: Cc1cc(=O)n2nc(N3CCc4ncc(-c5ccc(F)nc5)cc4C3)c(C)c(C)c2n1] | 417.2 |
| 1-205 | 7-(3-(3,4-difluorophenyl)-7,8- dihydro-1,6-naphthyridin- 6(5H)-yl)-2,8,9-trimethyl-4H- pyrimido[1,2-b]pyridazin-4-one | 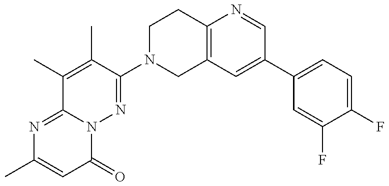 [SMILES: Cc1cc(=O)n2nc(N3CCc4ncc(-c5ccc(F)c(F)c5)cc4C3)c(C)c(C)c2n1] | 434.2 |
| 1-206 | 7-(3-(3-fluoro-4-methylphenyl)-7,8- dihydro-1,6-naphthyridin- 6(5H)-yl)-2,8-dimethyl-4H- pyrimido[1,2-b]pyridazin-4-one | 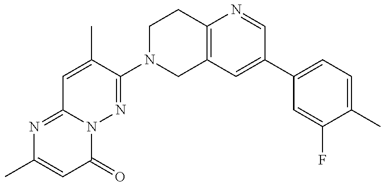 | 416.3 |
| 1-207 | 7-(3-(6-chloro-4-methylpyridin-3- yl)-7,8-dihydro-1,6-naphthyridin- 6(5H)-yl)-2,8-dimethyl-4H- pyrimido[1,2-b]pyridazin-4-one | 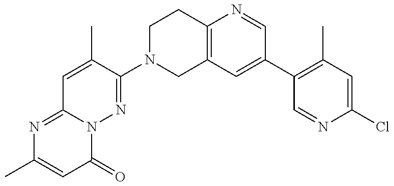 [SMILES: Cc1cc(=O)n2nc(N3CCc4ncc(-c5cnc(Cl)cc5C)cc4C3)c(C)cc2n1] | 433.2 |
| 1-208 | 2,8-dimethyl-7-(3-(2,4,5- trifluorophenyl)-7,8-dihydro-1,6- naphthyridin-6(5H)-yl)-4H- pyrimido[1,2-b]pyridazin-4-one | 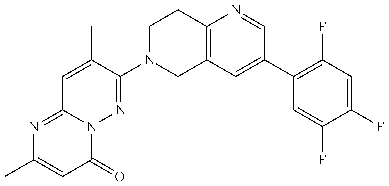 [SMILES: Cc1cc(=O)n2nc(N3CCc4ncc(-c5cc(F)c(F)cc5F)cc4C3)c(C)cc2n1] | 438.3 |
| 1-209 | 7-(3-(6-fluoro-4-methylpyridin-3- yl)-7,8-dihydro-1,6-naphthyridin- 6(5H)-yl)-2,8-dimethyl-4H- pyrimido[1,2-b]pyridazin-4-one | 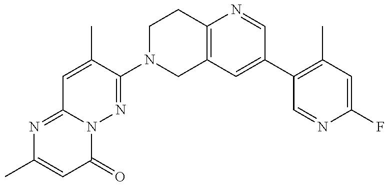 [SMILES: Cc1cc(=O)n2nc(N3CCc4ncc(-c5cnc(F)cc5C)cc4C3)c(C)cc2n1] | 417.4 |
| 1-210 | 7-(3-(5-fluoro-2-methylphenyl)-7,8- dihydro-1,6-naphthyridin- 6(5H)-yl)-2,8-dimethyl-4H- pyrimido[1,2-b]pyridazin-4-one | 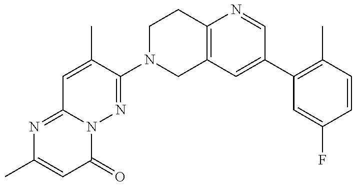 [SMILES: Cc1cc(=O)n2nc(N3CCc4ncc(-c5cc(F)ccc5C)cc4C3)c(C)cc2n1] | 416.4 |
| 1-211 | 7-(3-(3-fluorophenyl)-7,8-dihydro- 1,6-naphthyridin- 6(5H)-yl)-2,8-dimethyl-4H- pyrimido[1,2-b]pyridazin-4-one | 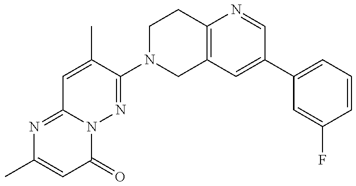 [SMILES: Cc1cc(=O)n2nc(N3CCc4ncc(-c5cccc(F)c5)cc4C3)c(C)cc2n1] | 423.9 |
| 1-212 | 7-(3-(6-fluoropyridin-3-yl)-7,8- dihydro-1,6-naphthyridin- 6(5H)-yl)-2,8-dimethyl-4H- pyrimido[1,2-b]pyridazin-4-one | 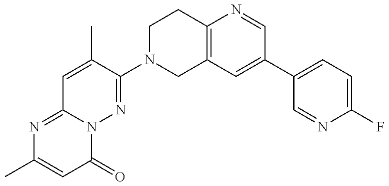 [SMILES: Cc1cc(=O)n2nc(N3CCc4ncc(-c5ccc(F)nc5)cc4C3)c(C)cc2n1] | 403.3 |
| 1-213 | 7-(3-(3,4-difluorophenyl)-7,8- dihydro-1,6-naphthyridin- 6(5H)-yl)-2,8-dimethyl-4H- pyrimido[1,2-b]pyridazin-4-one | 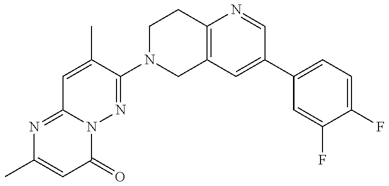 [SMILES: Cc1cc(=O)n2nc(N3CCc4ncc(-c5ccc(F)c(F)c5)cc4C3)c(C)cc2n1] | 420.3 |
| 1-214 | 2,8-dimethyl-7-(3-(4- methylpyridin-3-yl)-7,8-dihydro- 1,6-naphthyridin- 6(5H)-yl)-4H-pyrimido[1,2-b] pyridazin-4-one | 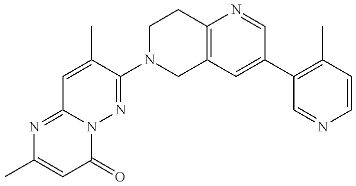 [SMILES: Cc1cc(=O)n2nc(N3CCc4ncc(-c5cnccc5C)cc4C3)c(C)cc2n1] | 399.5 |
| 1-215 | 7-(3-(1-(difluoromethyl)-1H- pyrazol-4-yl)-7,8-dihydro- 1,6-naphthyridin- 6(5H)-yl)-2,8-dimethyl-4H- pyrimido[1,2-b]pyridazin-4-one | 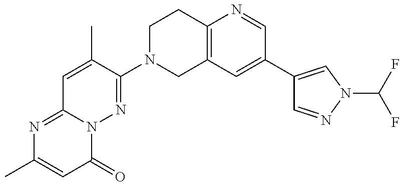 [SMILES: Cc1cc(=O)n2nc(N3CCc4ncc(-c5cnn(C(F)F)c5)cc4C3)c(C)cc2n1] | 424.4 |
| 1-216 | 7-(3-(3-fluoro-4-methoxyphenyl)- 7,8-dihydro-1,6-naphthyridin- 6(5H)-yl)-2,8-dimethyl-4H- pyrimido[1,2-b]pyridazin-4-one | 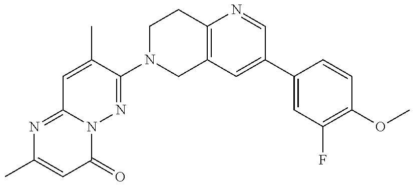 | 432.3 |
| 1-217 | 2,8-dimethyl-7-(3-(7- methylimidazo[1,2-a]pyridin-6-yl)- 7,8-dihydro-1,6-naphthyridin- 6(5H)-yl)-4H-pyrimido[1,2- b]pyridazin-4-one | 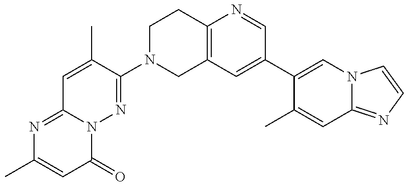 [SMILES: Cc1cc(=O)n2nc(N3CCc4ncc(-c5cn6ccnc6cc5C)cc4C3)c(C)cc2n1] | 438.3 |
| 1-218 | 2,8-dimethyl-7-(3-(1,3,5-trimethyl- 1H-pyrazol-4-yl)-7,8-dihydro- 1,6-naphthyridin- 6(5H)-yl)-4H-pyrimido[1,2-b] pyridazin-4-one | 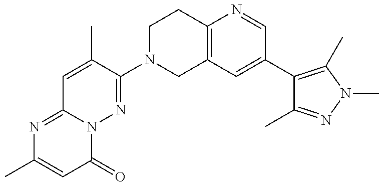 | 416.4 |
| 1-219 | 7-(3-(1-ethyl-1H-pyrazol-4-yl)-7,8- dihydro-1,6-naphthyridin- 6(5H)-yl)-2,8-dimethyl-4H- pyrimido[1,2-b]pyridazin-4-one | 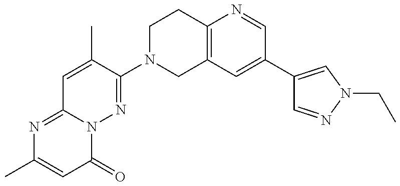 [SMILES: CCn1cc(-c2cnc3c(c2)CN(c2nn4c(=O)cc(C)nc4cc2C)CC3)cn1] | 402.4 |
| 1-220 | 7-(3-(imidazo[1,2-a]pyridin-6-yl)- 7,8-dihydro-1,6-naphthyridin- 6(5H)-yl)-2,8-dimethyl-4H- pyrimido[1,2-b]pyridazin-4-one | 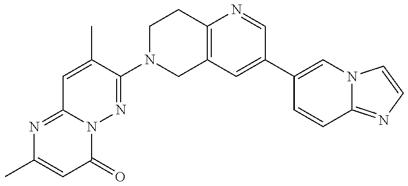 [SMILES: Cc1cc(=O)n2nc(N3CCc4ncc(-c5ccc6nccn6c5)cc4C3)c(C)cc2n1] | 424.4 |
| 1-221 | 7-(3-(6-(dimethylamino)pyridin-3- yl)-7,8-dihydro-1,6-naphthyridin- 6(5H)-yl)-2,8-dimethyl-4H- pyrimido[1,2-b]pyridazin-4-one | 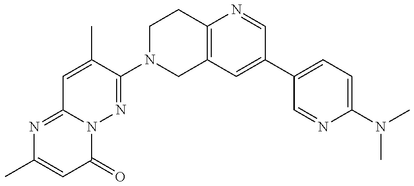 | 428.3 |
| 1-222 | 8,9-dimethyl-7-(3-(o-tolyl)-7,8- dihydro-1,6-naphthyridin- 6(5H)-yl)-4H-pyrimido[1,2-b] pyridazin-4-one | 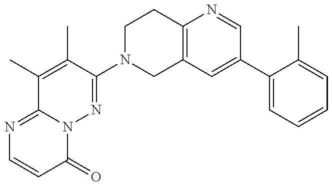 [SMILES: Cc1ccccc1-c1cnc2c(c1)CN(c1nn3c(=O)ccnc3c(C)c1C)CC2] | 398.4 |
| 1-223 | 5-(6-(8,9-dimethyl-4-oxo-4H- pyrimido[1,2-b]pyridazin-7-yl)- 5,6,7,8-tetrahydro-1,6-naphthyridin- 3-yl)nicotinonitrile | 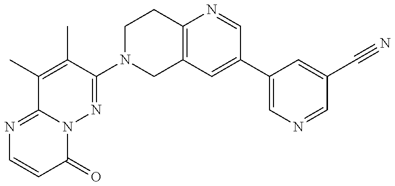 [SMILES: Cc1c(N2CCc3ncc(-c4cncc(C#N)c4)cc3C2)nn2c(=O)ccnc2c1C] | 410.4 |
| 1-224 | 8,9-dimethyl-7-(3-(quinolin-3-yl)- 7,8-dihydro-1,6-naphthyridin- 6(5H)-yl)-4H-pyrimido[1,2- b]pyridazin-4-one | 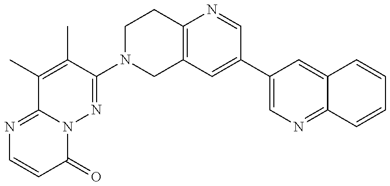 [SMILES: Cc1c(N2CCc3ncc(-c4cnc5ccccc5c4)cc3C2)nn2c(=O)ccnc2c1C] | 435.4 |
| 1-225 | 7-(3-(imidazo[1,2-a]pyridin-6-yl)- 7,8-dihydro-1,6-naphthyridin- 6(5H)-yl)-8,9-dimethyl-4H- pyrimido[1,2-b]pyridazin-4-one | 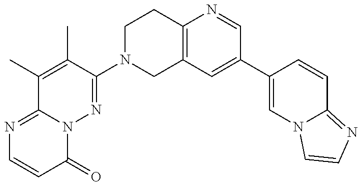 [SMILES: Cc1c(N2CCc3ncc(-c4ccc5nccn5c4)cc3C2)nn2c(=O)ccnc2c1C] | 424.3 |
| 1-226 | 2-(cyclopentylmethyl)-8,9- dimethyl-7-(3-(6-methylpyridin-3- yl)-7,8-dihydro-1,6-naphthyridin- 6(5H)-yl)-4H-pyrimido[1,2- b]pyridazin-4-one | 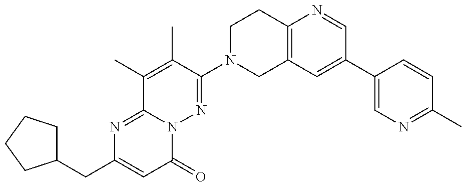 [SMILES: Cc1ccc(-c2cnc3c(c2)CN(c2nn4c(=O)cc(CC5CCCC5)nc4c(C)c2C)CC3)cn1] | 481.4 |
| 1-227 | 7-(3-(5-fluoro-2-methylphenyl)-7,8- dihydro-1,6-naphthyridin- 6(5H)-yl)-2-(methoxymethyl)-8,9- dimethyl-4H-pyrimido[1,2- b]pyridazin-4-one | 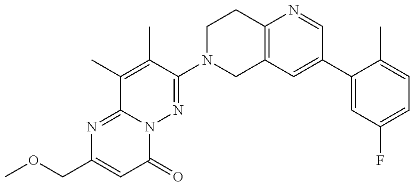 [SMILES: COCc1cc(=O)n2nc(N3CCc4ncc(-c5cc(F)ccc5C)cc4C3)c(C)c(C)c2n1] | 460.5 |
| 1-228 | 2-(methoxymethyl)-8,9-dimethyl-7- (3-(4-methylpyridin-3-yl)-7,8- dihydro-1,6-naphthyridin- 6(5H)-yl)-4H-pyrimido[1,2-b] pyridazin-4-one | 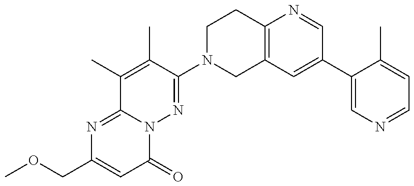 [SMILES: COCc1cc(=O)n2nc(N3CCc4ncc(-c5cnccc5C)cc4C3)c(C)c(C)c2n1] | 443.4 |
| 1-229 | 7-(3-(6-fluoro-4-methylpyridin-3- yl)-7,8-dihydro-1,6-naphthyridin- 6(5H)-yl)-2-(methoxymethyl)-8,9- dimethyl-4H-pyrimido[1,2- b]pyridazin-4-one | 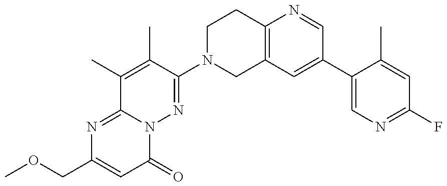 [SMILES: COCc1cc(=O)n2nc(N3CCc4ncc(-c5cnc(F)cc5C)cc4C3)c(C)c(C)c2n1] | 461.4 |
| 1-230 | 7-(3-(2-isopropylphenyl)-7,8- dihydro-1,6-naphthyridin- 6(5H)-yl)-2,8-dimethyl-4H- pyrimido[1,2-b]pyridazin-4-one | 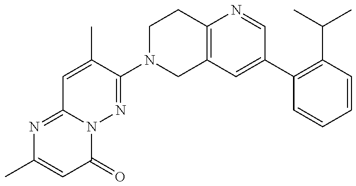 [SMILES: Cc1cc(=O)n2nc(N3CCc4ncc(-c5ccccc5C(C)C)cc4C3)c(C)cc2n1] | 426.4 |
| 1-231 | 7-(3-(2-cyclopropylphenyl)-7,8- dihydro-1,6-naphthyridin- 6(5H)-yl)-2,8-dimethyl-4H- pyrimido[1,2-b]pyridazin-4-one | 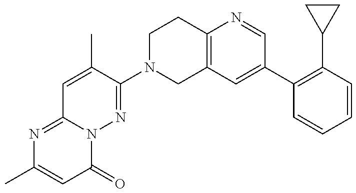 [SMILES: Cc1cc(=O)n2nc(N3CCc4ncc(-c5ccccc5C5CC5)cc4C3)c(C)cc2n1] | 424.4 |
| 1-232 | 7-(3-(2-ethylphenyl)-7,8-dihydro- 1,6-naphthyridin- 6(5H)-yl)-2,8-dimethyl-4H- pyrimido[1,2-b]pyridazin-4-one | 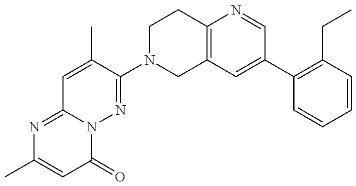 [SMILES: CCc1ccccc1-c1cnc2c(c1)CN(c1nn3c(=O)cc(C)nc3cc1C)CC2] | 412.5 |
| 1-233 | 7-(3-(2-methoxyphenyl)-7,8- dihydro-1,6-naphthyridin- 6(5H)-yl)-2,8-dimethyl-4H- pyrimido[1,2-b]pyridazin-4-one | 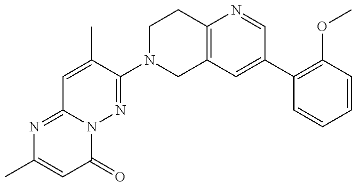 | 414.4 |
| 1-234 | 7-(3-(2-fluorophenyl)-7,8-dihydro- 1,6-naphthyridin- 6(5H)-yl)-2,8-dimethyl-4H- pyrimido[1,2-b]pyridazin-4-one | 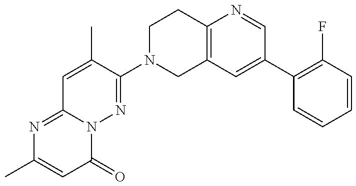 [SMILES: Cc1cc(=O)n2nc(N3CCc4ncc(-c5ccccc5F)cc4C3)c(C)cc2n1] | 402.4 |
| 1-235 | 7-(3-(2-hydroxyphenyl)-7,8- dihydro-1,6-naphthyridin- 6(5H)-yl)-2,8-dimethyl-4H- pyrimido[1,2-b]pyridazin-4-one | 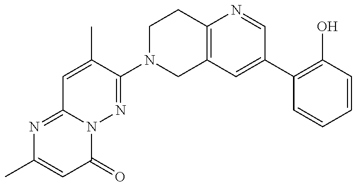 [SMILES: Cc1cc(=O)n2nc(N3CCc4ncc(-c5ccccc5O)cc4C3)c(C)cc2n1] | 400.3 |
| 1-236 | 7-(3-(2,5-difluorophenyl)-7,8- dihydro-1,6-naphthyridin- 6(5H)-yl)-2,8-dimethyl-4H- pyrimido[1,2-b]pyridazin-4-one | 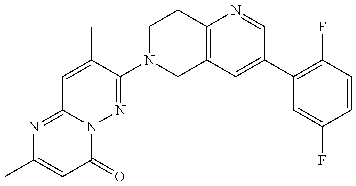 [SMILES: Cc1cc(=O)n2nc(N3CCc4ncc(-c5cc(F)ccc5F)cc4C3)c(C)cc2n1] | 420.3 |
| 1-237 | 2,8-dimethyl-7-(3-(o-tolyl)-7,8- dihydro-1,6-naphthyridin- 6(5H)-yl)-4H-pyrimido[1,2-b] pyridazin-4-one | 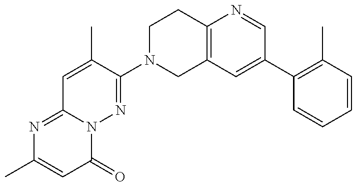 [SMILES: Cc1cc(=O)n2nc(N3CCc4ncc(-c5ccccc5C)cc4C3)c(C)cc2n1] | 398.5 |
| 1-238 | 7-(3-(2-fluoro-5-methylpyridin-4- yl)-7,8-dihydro-1,6-naphthyridin- 6(5H)-yl)-2,8-dimethyl-4H- pyrimido[1,2-b]pyridazin-4-one | 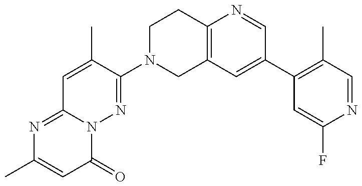 [SMILES: Cc1cc(=O)n2nc(N3CCc4ncc(-c5cc(F)ncc5C)cc4C3)c(C)cc2n1] | 417.4 |
| 1-239 | 2,8-dimethyl-7-(3-(2- methylpyridin-4-yl)-7,8-dihydro- 1,6-naphthyridin- 6(5H)-yl)-4H-pyrimido[1,2-b] pyridazin-4-one | 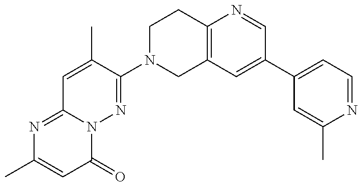 [SMILES: Cc1cc(-c2cnc3c(c2)CN(c2nn4c(=O)cc(C)nc4cc2C)CC3)ccn1] | 399.3 |
| 1-240 | 2,8-dimethyl-7-(3-(pyridin-4-yl)- 7,8-dihydro-1,6-naphthyridin- 6(5H)-yl)-4H-pyrimido[1,2- b]pyridazin-4-one | 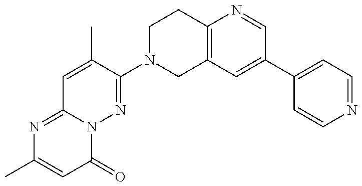 [SMILES: Cc1cc(=O)n2nc(N3CCc4ncc(-c5ccncc5)cc4C3)c(C)cc2n1] | 385.3 |
| 1-241 | 2,8-dimethyl-7-(3-(3- methylpyridin-4-yl)-7,8-dihydro- 1,6-naphthyridin- 6(5H)-yl)-4H-pyrimido[1,2-b] pyridazin-4-one | 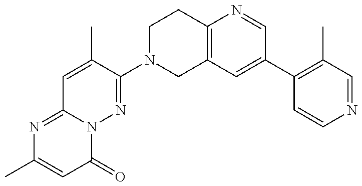 [SMILES: Cc1cc(=O)n2nc(N3CCc4ncc(-c5ccncc5C)cc4C3)c(C)cc2n1] | 399.4 |
| 1-242 | 7-(3-(2,6-difluoropyridin-4-yl)-7,8- dihydro-1,6-naphthyridin- 6(5H)-yl)-2,8-dimethyl-4H- pyrimido[1,2-b]pyridazin-4-one | 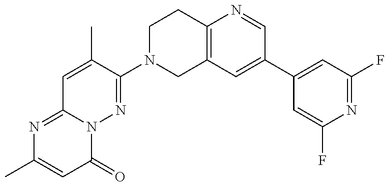 [SMILES: Cc1cc(=O)n2nc(N3CCc4ncc(-c5cc(F)nc(F)c5)cc4C3)c(C)cc2n1] | 421.4 |
| 1-243 | 2,8,9-trimethyl-7-(3-(2- methylpyridin-4-yl)-7,8-dihydro- 1,6-naphthyridin- 6(5H)-yl)-4H-pyrimido[1,2-b] pyridazin-4-one | 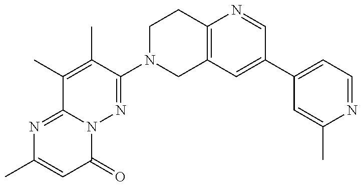 [SMILES: Cc1cc(-c2cnc3c(c2)CN(c2nn4c(=O)cc(C)nc4c(C)c2C)CC3)ccn1] | 413.4 |
| 1-244 | 2,8,9-trimethyl-7-(3-(pyridin-4-yl)- 7,8-dihydro-1,6-naphthyridin- 6(5H)-yl)-4H-pyrimido[1,2- b]pyridazin-4-one | 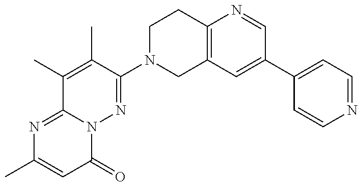 [SMILES: Cc1cc(=O)n2nc(N3CCc4ncc(-c5ccncc5)cc4C3)c(C)c(C)c2n1] | 399.3 |
| 1-245 | 2,8,9-trimethyl-7-(3-(3- methylpyridin-4-yl)-7,8-dihydro- 1,6-naphthyridin- 6(5H)-yl)-4H-pyrimido[1,2-b] pyridazin-4-one | 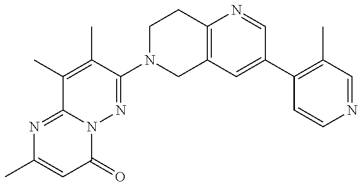 [SMILES: Cc1cc(=O)n2nc(N3CCc4ncc(-c5ccncc5C)cc4C3)c(C)c(C)c2n1] | 413.4 |
| 1-246 | 7-(3-(2,6-difluoropyridin-4-yl)-7,8- dihydro-1,6-naphthyridin- 6(5H)-yl)-2,8,9-trimethyl-4H- pyrimido[1,2-b]pyridazin-4-one | 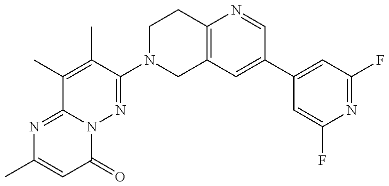 [SMILES: Cc1cc(=O)n2nc(N3CCc4ncc(-c5cc(F)nc(F)c5)cc4C3)c(C)c(C)c2n1] | 435.4 |
| 1-247 | 7-(3-(2-isopropylphenyl)-7,8- dihydro-1,6-naphthyridin- 6(5H)-yl)-2,8,9-trimethyl-4H- pyrimido[1,2-b]pyridazin-4-one | 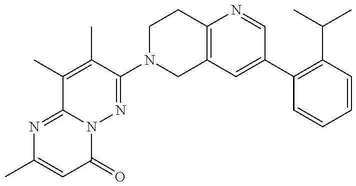 [SMILES: Cc1cc(=O)n2nc(N3CCc4ncc(-c5ccccc5C(C)C)cc4C3)c(C)c(C)c2n1] | 440.4 |
| 1-248 | 7-(3-(2-cyclopropylphenyl)-7,8- dihydro-1,6-naphthyridin- 6(5H)-yl)-2,8,9-trimethyl-4H- pyrimido[1,2-b]pyridazin-4-one | 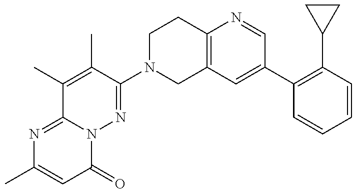 [SMILES: Cc1cc(=O)n2nc(N3CCc4ncc(-c5ccccc5C5CC5)cc4C3)c(C)c(C)c2n1] | 438.4 |
| 1-249 | 7-(3-(2-ethylphenyl)-7,8-dihydro- 1,6-naphthyridin- 6(5H)-yl)-2,8,9-trimethyl-4H- pyrimido[1,2-b]pyridazin-4-one | 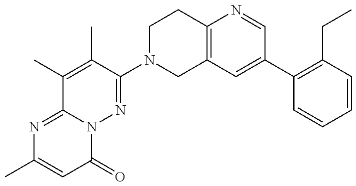 [SMILES: CCc1ccccc1-c1cnc2c(c1)CN(c1nn3c(=O)cc(C)nc3c(C)c1C)CC2] | 426.4 |
| 1-250 | 7-(3-(2-methoxyphenyl)-7,8- dihydro-1,6-naphthyridin- 6(5H)-yl)-2,8,9-trimethyl-4H- pyrimido[1,2-b]pyridazin-4-one | 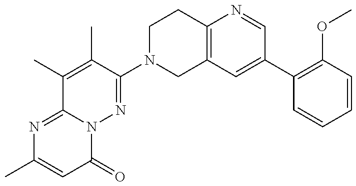 | 428.4 |
| 1-251 | 7-(3-(2-fluorophenyl)-7,8-dihydro- 1,6-naphthyridin- 6(5H)-yl)-2,8,9-trimethyl-4H- pyrimido[1,2-b]pyridazin-4-one | 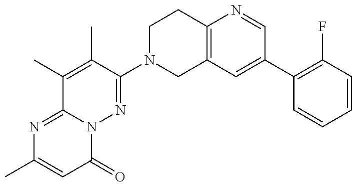 [SMILES: Cc1cc(=O)n2nc(N3CCc4ncc(-c5ccccc5F)cc4C3)c(C)c(C)c2n1] | 416.4 |
| 1-252 | 7-(3-(2,3-difluoro-5-methylphenyl)- 7,8-dihydro-1,6-naphthyridin- 6(5H)-yl)-2,8-dimethyl-4H- pyrimido[1,2-b]pyridazin-4-one | 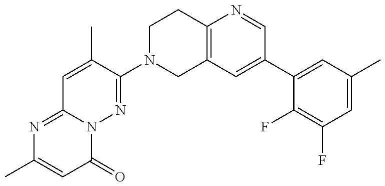 | 434.2 |
| 1-253 | 2,8-dimethyl-7-(3-(2,3,5- trifluorophenyl)-7,8-dihydro- 1,6-naphthyridin- 6(5H)-yl)-4H-pyrimido[1,2-b] pyridazin-4-one | 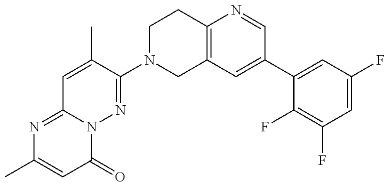 [SMILES: Cc1cc(=O)n2nc(N3CCc4ncc(-c5cc(F)cc(F)c5F)cc4C3)c(C)cc2n1] | 438.3 |
| 1-254 | 2,8-dimethyl-7-(3-(2- (trifluoromethoxy)phenyl)-7,8- dihydro-1,6-naphthyridin-6(5H)- yl)-4H-pyrimido[1,2-b]pyridazin- 4-one | 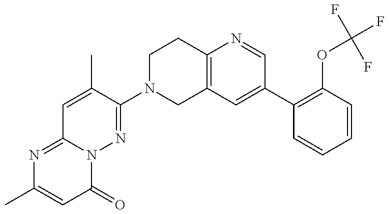 [SMILES: Cc1cc(=O)n2nc(N3CCc4ncc(-c5ccccc5OC(F)(F)F)cc4C3)c(C)cc2n1] | 468.2 |
| 1-255 | 2,8-dimethyl-7-(3-(2- (trifluoromethyl)phenyl)-7,8- dihydro-1,6-naphthyridin- 6(5H)-yl)-4H-pyrimido[1,2-b] pyridazin-4-one | 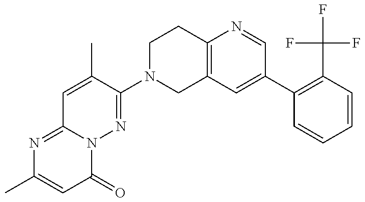 [SMILES: Cc1cc(=O)n2nc(N3CCc4ncc(-c5ccccc5C(F)(F)F)cc4C3)c(C)cc2n1] | 452.3 |
| 1-256 | 7-(3-(2-chlorophenyl)-7,8-dihydro- 1,6-naphthyridin- 6(5H)-yl)-2,8-dimethyl-4H- pyrimido[1,2-b]pyridazin-4-one | 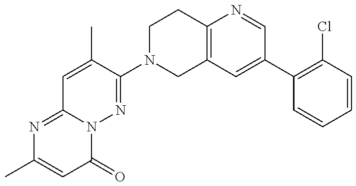 [SMILES: Cc1cc(=O)n2nc(N3CCc4ncc(-c5ccccc5Cl)cc4C3)c(C)cc2n1] | 418.3 |
| 1-257 | 7-(3-(2,4-difluorophenyl)-7,8- dihydro-1,6-naphthyridin-6(5H)- yl)-2,8-dimethyl-4H-pyrimido[1,2- b]pyridazin-4-one | 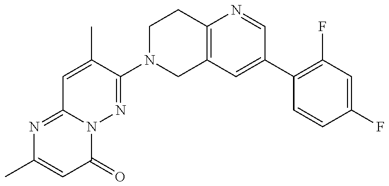 [SMILES: Cc1cc(=O)n2nc(N3CCc4ncc(-c5ccc(F)cc5F)cc4C3)c(C)cc2n1] | 420.4 |
| 1-258 | 7-(3-(2,5-difluoropyridin-4-yl)-7,8- dihydro-1,6-naphthyridin- 6(5H)-yl)-2,8-dimethyl-4H- pyrimido[1,2-b]pyridazin-4-one | 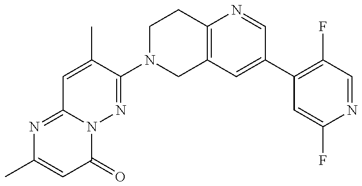 [SMILES: Cc1cc(=O)n2nc(N3CCc4ncc(-c5cc(F)ncc5F)cc4C3)c(C)cc2n1] | 421.3 |
| 1-259 | 7-(3-(3-methoxypyridin-4-yl)-7,8- 1,6-naphthyridin- 6(5H)-yl)-2,8-dimethyl-4H- pyrimido[1,2-b]pyridazin-4-one | 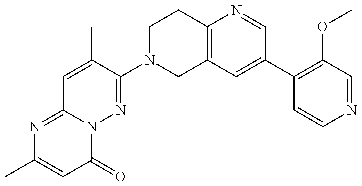 | 415.4 |
| 1-260 | 2,8-dimethyl-7-(3-(pyrazin-2-yl)- 7,8-dihydro-1,6-naphthyridin- 6(5H)-yl)-4H-pyrimido[1,2- b]pyridazin-4-one | 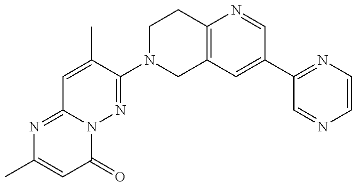 [SMILES: Cc1cc(=O)n2nc(N3CCc4ncc(-c5cnccn5)cc4C3)c(C)cc2n1] | 386.2 |
| 1-261 | 2-(methoxymethyl)-8,9-dimethyl-7- (3-(pyrazin-2-yl)-7,8-dihydro-1,6- naphthyridin-6(5H)-yl)-4H- pyrimido[1,2-b]pyridazin-4-one | 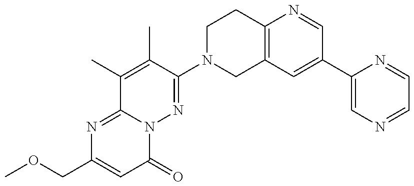 [SMILES: COCc1cc(=O)n2nc(N3CCc4ncc(-c5cnccn5)cc4C3)c(C)c(C)c2n1] | 430.3 |
| 1-262 | 7-(3-(6-methoxy-4-methylpyridin- 3-yl)-7,8-dihydro-1,6-naphthyridin- 6(5H)-yl)-2-(methoxymethyl)-8,9- dimethyl-4H-pyrimido[1,2- b]pyridazin-4-one | 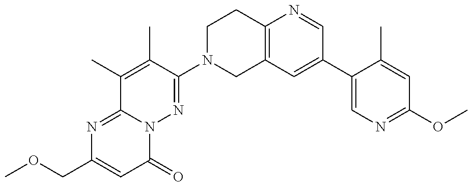 | 473.4 |
| 1-263 | 7-(3-(1-cyclopropyl-1H-pyrazol-4- yl)-7,8-dihydro-1,6-naphthyridin- 6(5H)-yl)-2-(methoxymethyl)-8,9- dimethyl-4H-pyrimido[1,2- b]pyridazin-4-one | 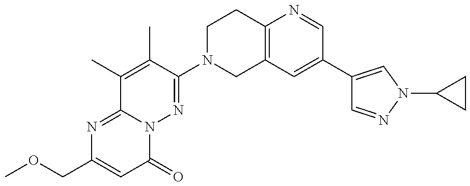 [SMILES: COCc1cc(=O)n2nc(N3CCc4ncc(-c5cnn(C6CC6)c5)cc4C3)c(C)c(C)c2n1] | 458.5 |
| 1-264 | 7-(3-(4-fluoro-2-methylphenyl)- 7,8-dihydro-1,6-naphthyridin- 6(5H)-yl)-8,9- dimethyl-4H-pyrimido[1,2- b]pyridazin-4-one | 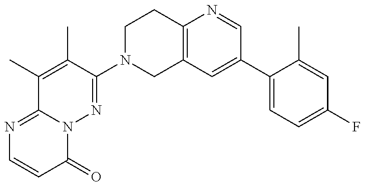 [SMILES: Cc1cc(F)ccc1-c1cnc2c(c1)CN(c1nn3c(=O)ccnc3c(C)c1C)CC2] | 416.3 |
| 1-265 | 8,9-dimethyl-7-(3-(2- (trifluoromethoxy)phenyl)-7,8- dihydro-1,6-naphthyridin- 6(5H)-yl)-4H-pyrimido[1,2-b] pyridazin-4-one | 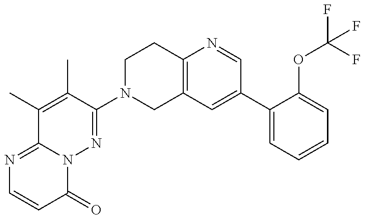 [SMILES: Cc1c(N2CCc3ncc(-c4ccccc4OC(F)(F)F)cc3C2)nn2c(=O)ccnc2c1C] | 468.4 |
| 1-266 | 8,9-dimethyl-7-(3-(2- (trifluoromethyl)phenyl)-7,8- dihydro-1,6-naphthyridin- 6(5H)-yl)-4H-pyrimido[1,2-b] pyridazin-4-one | 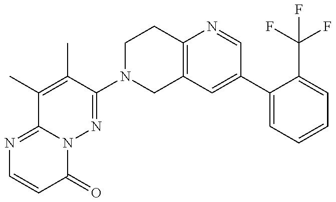 [SMILES: Cc1c(N2CCc3ncc(-c4ccccc4C(F)(F)F)cc3C2)nn2c(=O)ccnc2c1C] | 452.4 |
| 1-267 | 7-(3-(2-chlorophenyl)-7,8-dihydro- 1,6-naphthyridin- 6(5H)-yl)-8,9-dimethyl-4H- pyrimido[1,2-b]pyridazin-4-one | 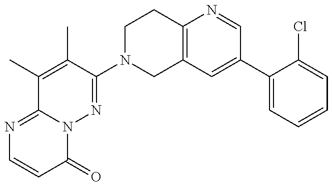 [SMILES: Cc1c(N2CCc3ncc(-c4ccccc4Cl)cc3C2)nn2c(=O)ccnc2c1C] | 418 |
| 1-268 | 7-(3-(2,5-difluoropyridin-4-yl)-7,8- dihydro-1,6-naphthyridin-6(5H)- yl)-8,9-dimethyl-4H-pyrimido[1,2- b]pyridazin-4-one | 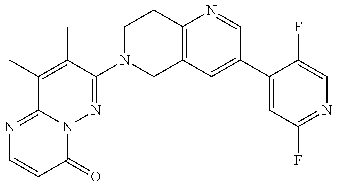 [SMILES: Cc1c(N2CCc3ncc(-c4cc(F)ncc4F)cc3C2)nn2c(=O)ccnc2c1C] | 421.2 |
| 1-269 | 7-(3-(3-methoxypyridin-4-yl)-7,8- dihydro-1,6-naphthyridin-6(5H)- yl)-8,9-dimethyl-4H-pyrimido[1,2- b]pyridazin-4-one | 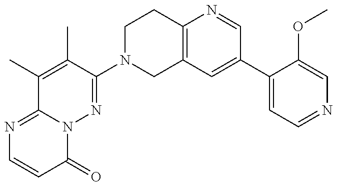 [SMILES: COc1cnccc1-c1cnc2c(c1)CN(c1nn3c(=O)ccnc3c(C)c1C)CC2] | 415.2 |
| 1-270 | 8-(methoxymethyl)-9-methyl-7-(3- (trifluoromethyl)-7,8-dihydro-1,6- naphthyridin-6(5H)-yl)-4H- pyrimido[1,2-b]pyridazin-4-one | 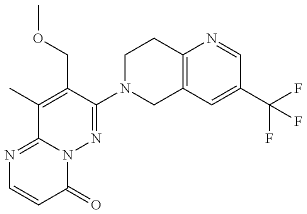 [SMILES: COCc1c(N2CCc3ncc(C(F)(F)F)cc3C2)nn2c(=O)ccnc2c1C] | 406.3 |
| 1-271 | 8-(methoxymethyl)-9-methyl-7-(3- (6-methylpyridin-3-yl)-7,8-dihydro- 1,6-naphthyridin- 6(5H)-yl)-4H-pyrimido[1,2-b] pyridazin-4-one | 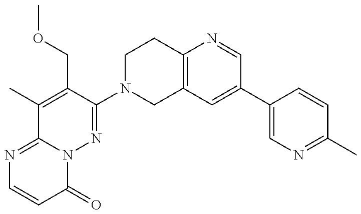 [SMILES: COCc1c(N2CCc3ncc(-c4ccc(C)nc4)cc3C2)nn2c(=O)ccnc2c1C] | 429.4 |
| 1-272 | 7-(3-(2,3-difluoro-5-methylphenyl)- 7,8-dihydro-1,6-naphthyridin- 6(5H)-yl)-2,8,9-trimethyl-4H- pyrimido[1,2-b]pyridazin-4-one | 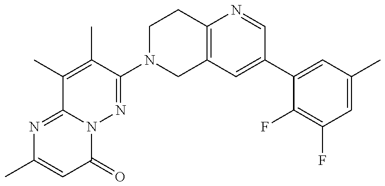 | 448.4 |
| 1-273 | 2,8,9-trimethyl-7-(3-(2,3,5- trifluorophenyl)-7,8-dihydro-1,6- naphthyridin-6(5H)-yl)-4H- pyrimido[1,2-b]pyridazin-4-one | 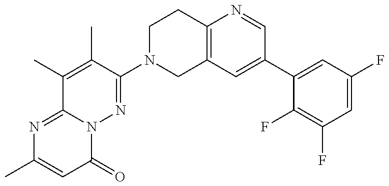 [SMILES: Cc1cc(=O)n2nc(N3CCc4ncc(-c5cc(F)cc(F)c5F)cc4C3)c(C)c(C)c2n1] | 452.4 |
| 1-274 | 7-(3-(4-fluoro-2-methylphenyl)-7,8- dihydro-1,6-naphthyridin- 6(5H)-yl)-2,8,9-trimethyl-4H- pyrimido[1,2-b]pyridazin-4-one | 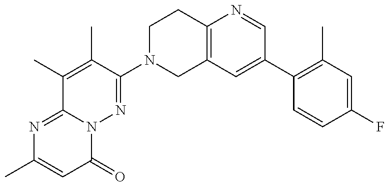 [SMILES: Cc1cc(=O)n2nc(N3CCc4ncc(-c5ccc(F)cc5C)cc4C3)c(C)c(C)c2n1] | 430.3 |
| 1-275 | 2,8,9-trimethyl-7-(3-(2- (trifluoromethoxy)phenyl)-7,8- dihydro-1,6-naphthyridin-6(5H)- yl)-4H-pyrimido[1,2-b]pyridazin-4- one | 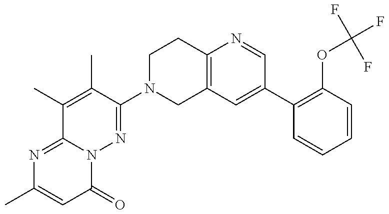 [SMILES: Cc1cc(=O)n2nc(N3CCc4ncc(-c5ccccc5OC(F)(F)F)cc4C3)c(C)c(C)c2n1] | 482.2 |
| 1-276 | 9-(methoxymethyl)-8-methyl-7-(3- (trifluoromethyl)-7,8-dihydro-1,6- naphthyridin-6(5H)-yl)-4H- pyrimido[1,2-b]pyridazin-4-one | 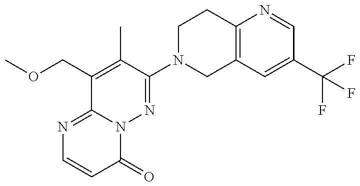 [SMILES: COCc1c(C)c(N2CCc3ncc(C(F)(F)F)cc3C2)nn2c(=O)ccnc12] | 406.3 |
| 1-277 | 2,8,9-trimethyl-7-(3-(2- (trifluoromethyl)phenyl)-7,8- dihydro-1,6-naphthyridin- 6(5H)-yl)-4H-pyrimido[1,2-b] pyridazin-4-one | 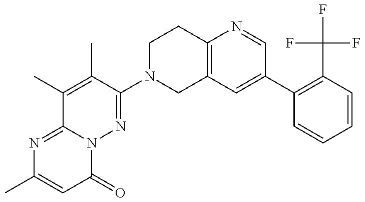 [SMILES: Cc1cc(=O)n2nc(N3CCc4ncc(-c5ccccc5C(F)(F)F)cc4C3)c(C)c(C)c2n1] | 466.2 |
| 1-278 | 7-(3-(2-chlorophenyl)-7,8-dihydro- 1,6-naphthyridin- 6(5H)-yl)-2,8,9-trimethyl-4H- pyrimido[1,2-b]pyridazin-4-one | 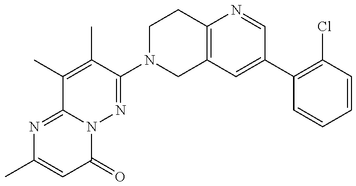 [SMILES: Cc1cc(=O)n2nc(N3CCc4ncc(-c5ccccc5Cl)cc4C3)c(C)c(C)c2n1] | 432.2 |
| 1-279 | 7-(3-(2,4-difluorophenyl)-7,8- dihydro-1,6-naphthyridin- 6(5H)-yl)-2,8,9-trimethyl-4H- pyrimido[1,2-b]pyridazin-4-one | 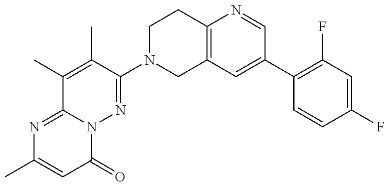 [SMILES: Cc1cc(=O)n2nc(N3CCc4ncc(-c5ccc(F)cc5F)cc4C3)c(C)c(C)c2n1] | 434.3 |
| 1-280 | 7-(3-(2-isopropoxyphenyl)-7,8- dihydro-1,6-naphthyridin- 6(5H)-yl)-2,8,9-trimethyl-4H- pyrimido[1,2-b]pyridazin-4-one | 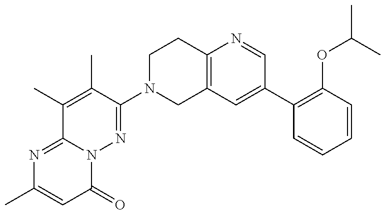 [SMILES: Cc1cc(=O)n2nc(N3CCc4ncc(-c5ccccc5OC(C)C)cc4C3)c(C)c(C)c2n1] | 456.4 |
| 1-281 | 7-(3-(2,5-difluoropyridin-4-yl)-7,8- dihydro-1,6-naphthyridin- 6(5H)-yl)-2,8,9-trimethyl-4H- pyrimido[1,2-b]pyridazin-4-one | 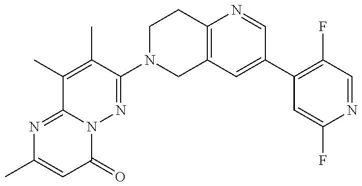 [SMILES: Cc1cc(=O)n2nc(N3CCc4ncc(-c5cc(F)ncc5F)cc4C3)c(C)c(C)c2n1] | 435.3 |
| 1-282 | 7-(3-(3-methoxypyridin-4-yl)-7,8- dihydro-1,6-naphthyridin- 6(5H)-yl)-2,8,9-trimethyl-4H- pyrimido[1,2-b]pyridazin-4-one | 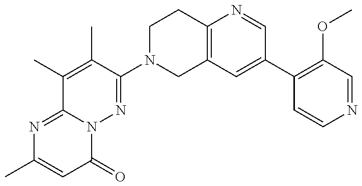 | 429.4 |
| 1-283 | 2-(difluoromethyl)-7-(3-(3,4- difluorophenyl)-7,8-dihydro-1,6- naphthyridin-6(5H)-yl)-8,9- dimethyl-4H-pyrimido[1,2- b]pyridazin-4-one | 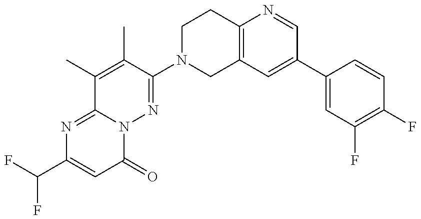 [SMILES: Cc1c(N2CCc3ncc(-c4ccc(F)c(F)c4)cc3C2)nn2c(=O)cc(C(F)F)nc2c1C] | 470.3 |
| 1-284 | 7-(3-(2,3-difluoro-5-methylphenyl)- 7,8-dihydro-1,6-naphthyridin- 6(5H)-yl)-2-(difluoromethyl)-8,9- dimethyl-4H-pyrimido[1,2-b] pyridazin-4-one | 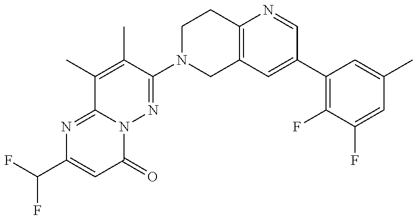 [SMILES: Cc1cc(F)c(F)c(-c2cnc3c(c2)CN(c2nn4c(=O)cc(C(F)F)nc4c(C)c2C)CC3)c1] | 484.2 |
| 1-285 | 7-(3-(6-methoxy-4-methylpyridin- 3-yl)-7,8-dihydro-1,6-naphthyridin- 6(5H)-yl)-2,8-dimethyl-4H- pyrimido[1,2-b]pyridazin-4-one | 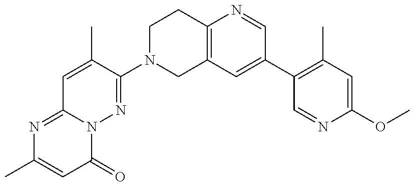 | 429.2 |
| 1-286 | 2,8-dimethyl-7-(3-(pyrimidin-5-yl)- 7,8-dihydro-1,6-naphthyridin- 6(5H)-yl)-4H-pyrimido[1,2- b]pyridazin-4-one | 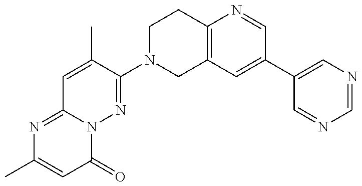 [SMILES: Cc1cc(=O)n2nc(N3CCc4ncc(-c5cncnc5)cc4C3)c(C)cc2n1] | 386.2 |
| 1-287 | 2,8-dimethyl-7-(3-(1-(oxetan-3-yl)- 1H-pyrazol-4-yl)-7,8-dihydro-1,6- naphthyridin-6(5H)-yl)-4H- pyrimido[1,2-b]pyridazin-4-one | 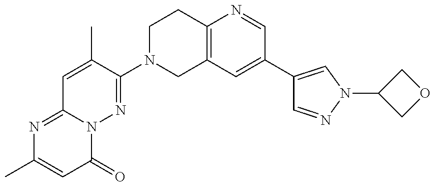 [SMILES: Cc1cc(=O)n2nc(N3CCc4ncc(-c5cnn(C6COC6)c5)cc4C3)c(C)cc2n1] | 430.2 |
| 1-288 | 2,8-dimethyl-7-(3-(pyrazolo[1,5- a]pyridin-3-yl)-7,8-dihydro- 1,6-naphthyridin- 6(5H)-yl)-4H-pyrimido[1,2-b] pyridazin-4-one | 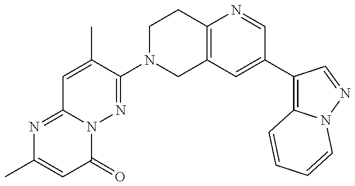 [SMILES: Cc1cc(=O)n2nc(N3CCc4ncc(-c5cnn6ccccc56)cc4C3)c(C)cc2n1] | 424.2 |
| 1-289 | 2,8-dimethyl-7-(3-(5- methylpyridin-3-yl)-7,8-dihydro- 1,6-naphthyridin- 6(5H)-yl)-4H-pyrimido[1,2-b] pyridazin-4-one | 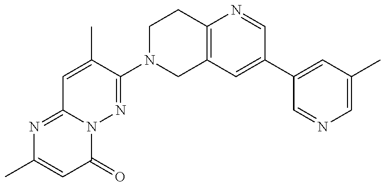 | 399.2 |
| 1-290 | 2,8-dimethyl-7-(3-(pyridin-3-yl)- 7,8-dihydro-1,6-naphthyridin- 6(5H)-yl)-4H-pyrimido[1,2- b]pyridazin-4-one | 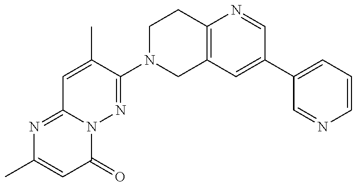 [SMILES: Cc1cc(=O)n2nc(N3CCc4ncc(-c5cccnc5)cc4C3)c(C)cc2n1] | 385.2 |
| 1-291 | 2,8-dimethyl-7-(3-(1-(2,2,2- trifluoroethyl)-1H-pyrazol-4-yl)- 7,8-dihydro-1,6-naphthyridin- 6(5H)-yl)-4H-pyrimido[1,2- b]pyridazin-4-one | 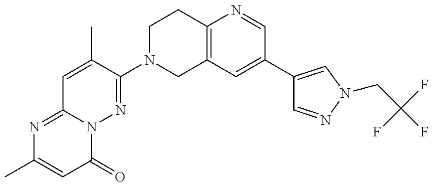 [SMILES: Cc1cc(=O)n2nc(N3CCc4ncc(-c5cnn(CC(F)(F)F)c5)cc4C3)c(C)cc2n1] | 456.2 |
| 1-292 | 7-(3-(2,4-dimethylphenyl)-7,8- dihydro-1,6-naphthyridin-6(5H)- yl)-2,8-dimethyl-4H-pyrimido[1,2- b]pyridazin-4-one | 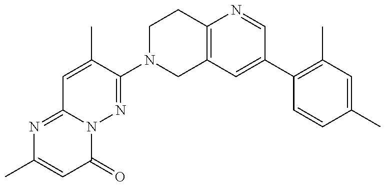 | 412.2 |
| 1-293 | 2,8-dimethyl-7-(3-(p-tolyl)-7,8- dihydro-1,6-naphthyridin-6(5H)- yl)-4H-pyrimido[1,2-b]pyridazin- 4-one | 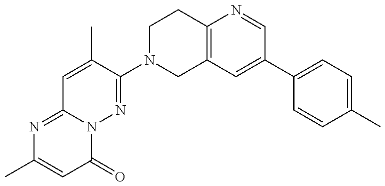 | 398.2 |
| 1-294 | 7-(3-(1,5-dimethyl-1H-pyrazol-4- yl)-7,8-dihydro-1,6-naphthyridin- 6(5H)-yl)-2,8-dimethyl-4H- pyrimido[1,2-b]pyridazin-4-one | 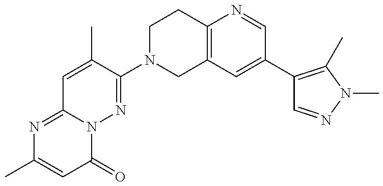 | 402.2 |
| 1-295 | 7-(3-(4-methoxypyridin-3-yl)-7,8- dihydro-1,6-naphthyridin-6(5H)- yl)-2,8-dimethyl-4H-pyrimido[1,2- b]pyridazin-4-one | 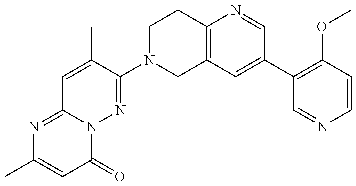 | 386 |
| 1-296 | 8,9-dimethyl-7-(3-(7- methylimidazo[1,2-a]pyridin-6-yl)- 7,8-dihydro-1,6-naphthyridin- 6(5H)-yl)-4H-pyrimido[1,2- b]pyridazin-4-one | 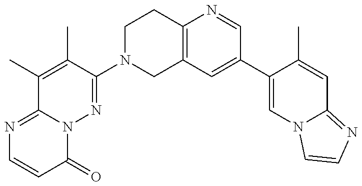 [SMILES: Cc1cc2nccn2cc1-c1cnc2c(c1)CN(c1nn3c(=O)ccnc3c(C)c1C)CC2] | 438.4 |
| 1-297 | 2,8-dimethyl-7-(3-(thiophen-2-yl)- 7,8-dihydro-1,6-naphthyridin- 6(5H)-yl)-4H-pyrimido[1,2- b]pyridazin-4-one | 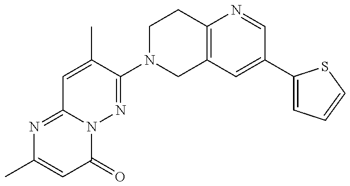 [SMILES: Cc1cc(=O)n2nc(N3CCc4ncc(-c5cccs5)cc4C3)c(C)cc2n1] | 390.2 |
| 1-298 | 7-(3-(3-methoxythiophen-2-yl)-7,8- dihydro-1,6-naphthyridin- 6(5H)-yl)-2,8-dimethyl-4H- pyrimido[1,2-b]pyridazin-4-one | 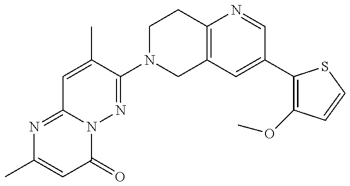 [SMILES: COc1ccsc1-c1cnc2c(c1)CN(c1nn3c(=O)cc(C)nc3cc1C)CC2] | 420.2 |
| 1-299 | 7-(3-(5-(hydroxymethyl)thiophen- 2-yl)-7,8-dihydro-1,6-naphthyridin- 6(5H)-yl)-2,8-dimethyl-4H- pyrimido[1,2-b]pyridazin-4-one | 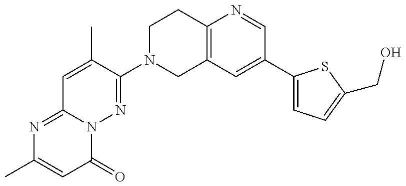 [SMILES: Cc1cc(=O)n2nc(N3CCc4ncc(-c5ccc(CO)s5)cc4C3)c(C)cc2n1] | 420 |
| 1-300 | 2,8-dimethyl-7-(3-(thiophen-3-yl)- 7,8-dihydro-1,6-naphthyridin- 6(5H)-yl)-4H-pyrimido[1,2- b]pyridazin-4-one | 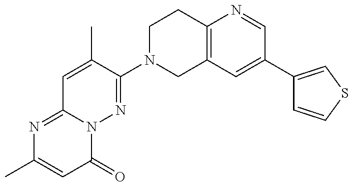 [SMILES: Cc1cc(=O)n2nc(N3CCc4ncc(-c5ccsc5)cc4C3)c(C)cc2n1] | 390.2 |
| 1-301 | 7-(3-(furan-2-yl)-7,8-dihydro-1,6- naphthyridin-6(5H)-yl)-2,8- dimethyl-4H-pyrimido[1,2- b]pyridazin-4-one | 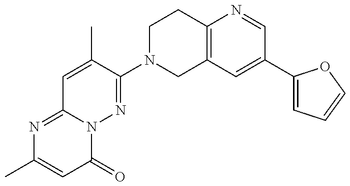 [SMILES: Cc1cc(=O)n2nc(N3CCc4ncc(-c5ccco5)cc4C3)c(C)cc2n1] | 374.2 |
| 1-302 | 2,8-dimethyl-7-(3-(5-methylfuran- 2-yl)-7,8-dihydro-1,6-naphthyridin- 6(5H)-yl)-4H-pyrimido[1,2-b] pyridazin-4-one | 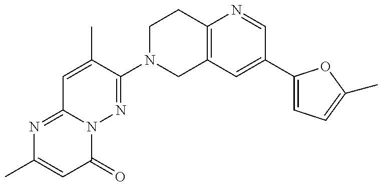 | 388.2 |
| 1-303 | 7-(3-(3-methoxythiophen-2-yl)-7,8- dihydro-1,6-naphthyridin- 6(5H)-yl)-2,8,9-trimethyl-4H- pyrimido[1,2-b]pyridazin-4-one | 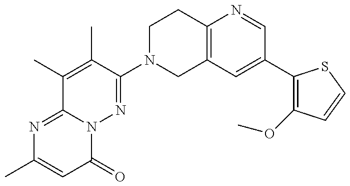 [SMILES: COc1ccsc1-c1cnc2c(c1)CN(c1nn3c(=O)cc(C)nc3c(C)c1C)CC2] | 434.3 |
| 1-304 | 2,8,9-trimethyl-7-(3-(thiophen-3- yl)-7,8-dihydro-1,6-naphthyridin- 6(5H)-yl)-4H-pyrimido[1,2- b]pyridazin-4-one | 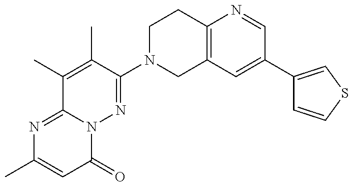 [SMILES: Cc1cc(=O)n2nc(N3CCc4ncc(-c5ccsc5)cc4C3)c(C)c(C)c2n1] | 404.4 |
| 1-305 | 2,8,9-trimethyl-7-(3-(5- methylfuran-2-yl)-7,8-dihydro- 1,6-naphthyridin- 6(5H)-yl)-4H-pyrimido[1,2-b] pyridazin-4-one | 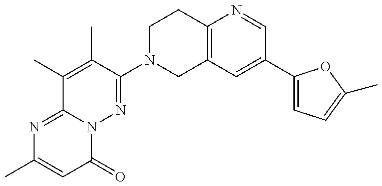 | 402.5 |
| 1-306 | 2-(difluoromethyl)-7-(3-(3- fluorophenyl)-7,8-dihydro-1,6- naphthyridin-6(5H)-yl)-8,9- dimethyl-4H-pyrimido[1,2- b]pyridazin-4-one | 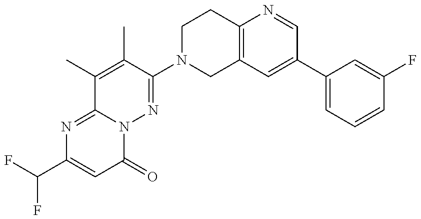 [SMILES: Cc1c(N2CCc3ncc(-c4cccc(F)c4)cc3C2)nn2c(=O)cc(C(F)F)nc2c1C] | 452.4 |
| 1-307 | 2-(difluoromethyl)-8,9-dimethyl-7- (3-(3-methylpyridin-4-yl)-7,8- dihydro-1,6-naphthyridin-6(5H)- yl)-4H-pyrimido[1,2-b]pyridazin- 4-one | 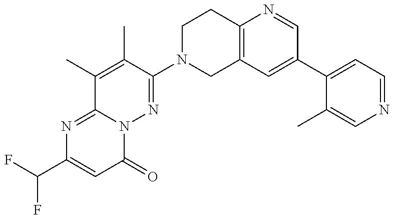 [SMILES: Cc1cnccc1-c1cnc2c(c1)CN(c1nn3c(=O)cc(C(F)F)nc3c(C)c1C)CC2] | 449.4 |
| 1-308 | 2-(difluoromethyl)-7-(3-(1-(2- (dimethylamino)ethyl)-1H-pyrazol- 4-yl)-7,8-dihydro-1,6-naphthyridin- 6(5H)-yl)-8,9-dimethyl-4H- pyrimido[1,2-b]pyridazin-4-one | 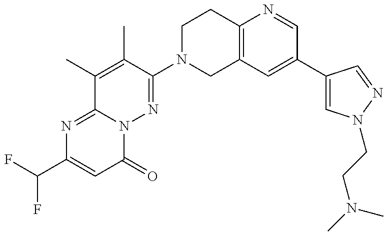 [SMILES: Cc1c(N2CCc3ncc(-c4cnn(CCN(C)C)c4)cc3C2)nn2c(=O)cc(C(F)F)nc2c1C] | 495.4 |
| 1-309 | 2-(difluoromethyl)-8,9-dimethyl-7- (3-(1-(oxetan-3-yl)-1H-pyrazol-4- yl)-7,8-dihydro-1,6-naphthyridin- 6(5H)-yl)-4H-pyrimido[1,2- b]pyridazin-4-one | 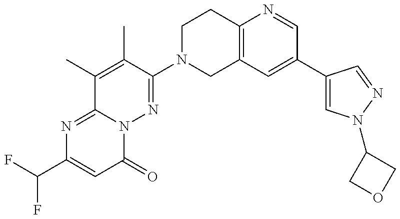 [SMILES: Cc1c(N2CCc3ncc(-c4cnn(C5COC5)c4)cc3C2)nn2c(=O)cc(C(F)F)nc2c1C] | 480.4 |
| 1-310 | 2-(difluoromethyl)-8,9-dimethyl-7- (3-(1,3,5-trimethyl-1H-pyrazol-4- yl)-7,8-dihydro-1,6-naphthyridin- 6(5H)-yl)-4H-pyrimido[1,2- b]pyridazin-4-one | 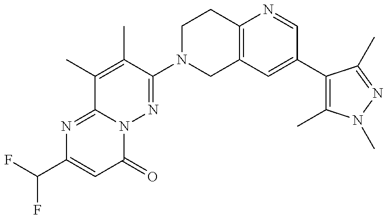 [SMILES: Cc1nn(C)c(C)c1-c1cnc2c(c1)CN(c1nn3c(=O)cc(C(F)F)nc3c(C)c1C)CC2] | 466.4 |
| 1-311 | 2-(difluoromethyl)-7-(3-(3- methoxythiophen-2-yl)-7,8- dihydro-1,6-naphthyridin- 6(5H)-yl)-8,9-dimethyl-4H- pyrimido[1,2-b]pyridazin-4-one | 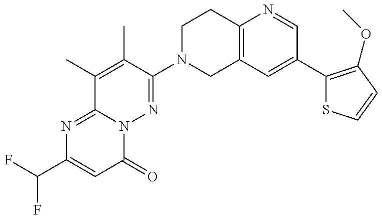 [SMILES: COc1ccsc1-c1cnc2c(c1)CN(c1nn3c(=O)cc(C(F)F)nc3c(C)c1C)CC2] | 470.3 |
| 1-312 | 2-(difluoromethyl)-8,9-dimethyl-7- (3-(thiophen-2-yl)-7,8-dihydro-1,6- naphthyridin-6(5H)-yl)-4H- pyrimido[1,2-b]pyridazin-4-one | 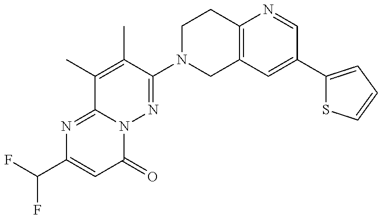 [SMILES: Cc1c(N2CCc3ncc(-c4cccs4)cc3C2)nn2c(=O)cc(C(F)F)nc2c1C] | 440.3 |
| 1-313 | 2-(methoxymethyl)-8-methyl-7-(3- (trifluoromethyl)-7,8-dihydro-1,6- naphthyridin-6(5H)-yl)-4H- pyrimido[1,2-b]pyridazin-4-one | 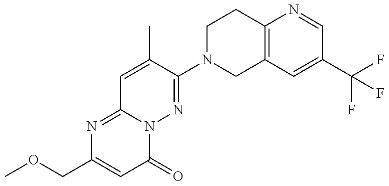 [SMILES: COCc1cc(=O)n2nc(N3CCc4ncc(C(F)(F)F)cc4C3)c(C)cc2n1] | 406.2 |
| 1-314 | 2-(methoxymethyl)-8-methyl-7-(3- (6-methylpyridin-3-yl)-7,8-dihydro- 1,6-naphthyridin-6(5H)-yl)-4H- pyrimido[1,2-b]pyridazin-4-one | 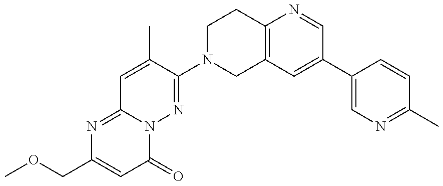 | 429.2 |
| 1-315 | 7-(3-(6-(difluoromethoxy)pyridin- 3-yl)-7,8-dihydro-1,6-naphthyridin- 6(5H)-yl)-8-methyl-4H- pyrimido[1,2-b]pyridazin-4-one | 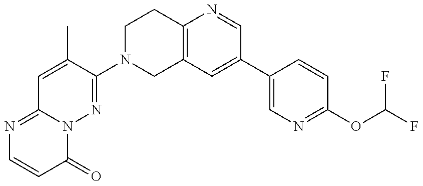 [SMILES: Cc1cc2nccc(=O)n2nc1N1CCc2ncc(-c3ccc(OC(F)F)nc3)cc2C1] | 437.2 |
| 1-316 | 5-(6-(8-methyl-4-oxo-4H- pyrimido[1,2-b]pyridazin-7-yl)- 5,6,7,8-tetrahydro-1,6-naphthyridin- 3-yl)picolinonitrile | 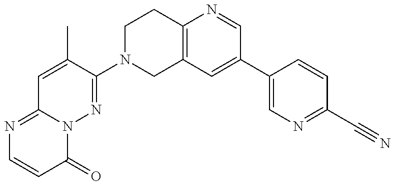 [SMILES: Cc1cc2nccc(=O)n2nc1N1CCc2ncc(-c3ccc(C#N)nc3)cc2C1] | 396.3 |
| 1-317 | 7-(3-(6-(difluoromethoxy)-4- methylpyridin-3-yl)-7,8-dihydro- 1,6-naphthyridin-6(5H)-yl)-8- methyl-4H-pyrimido[1,2- b]pyridazin-4-one | 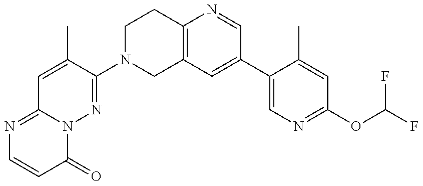 [SMILES: Cc1cc(OC(F)F)ncc1-c1cnc2c(c1)CN(c1nn3c(=O)ccnc3cc1C)CC2] | 451.4 |
| 1-318 | 7-(3-(2,6-difluoropyridin-3-yl)-7,8- dihydro-1,6-naphthyridin- 6(5H)-yl)-8-methyl-4H-pyrimido [1,2-b]pyridazin-4-one | 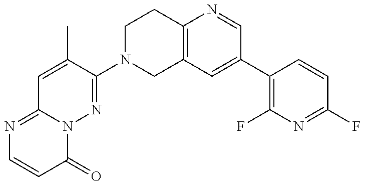 [SMILES: Cc1cc2nccc(=O)n2nc1N1CCc2ncc(-c3ccc(F)nc3F)cc2C1] | 407.4 |
| 1-319 | 3-(6-(8-methyl-4-oxo-4H- pyrimido[1,2-b]pyridazin-7-yl)- 5,6,7,8-tetrahydro-1,6-naphthyridin- 3-yl)isonicotinonitrile | 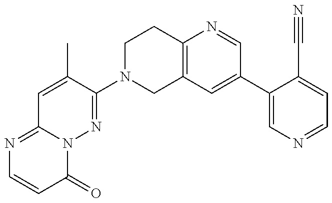 [SMILES: Cc1cc2nccc(=O)n2nc1N1CCc2ncc(-c3cnccc3C#N)cc2C1] | 396.4 |
| 1-320 | 7-(3-(2,5-difluoropyridin-4-yl)-7,8- dihydro-1,6-naphthyridin- 6(5H)-yl)-2-(methoxymethyl)-8- methyl-4H-pyrimido[1,2-b] pyridazin-4-one | 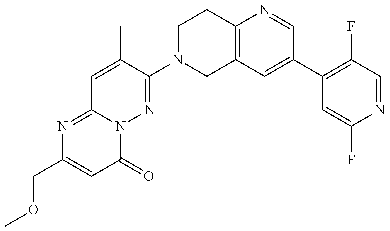 [SMILES: COCc1cc(=O)n2nc(N3CCc4ncc(-c5cc(F)ncc5F)cc4C3)c(C)cc2n1] | 451 |
| 1-321 | 8-methyl-7-(3-(1-methyl-1H-1,2,3- triazol-4-yl)-7,8-dihydro-1,6- naphthyridin-6(5H)-yl)-4H- pyrimido[1,2-b]pyridazin-4-one | 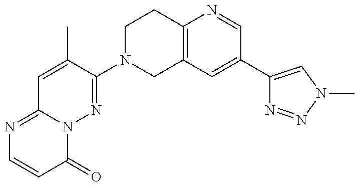 [SMILES: Cc1cc2nccc(=O)n2nc1N1CCc2ncc(-c3cn(C)nn3)cc2C1] | 375.4 |
| 1-322 | 7-(3-(6-fluoropyridin-3-yl)-7,8- dihydro-1,6-naphthyridin- 6(5H)-yl)-2-(methoxymethyl)-8- methyl-4H-pyrimido[1,2-b] pyridazin-4-one | 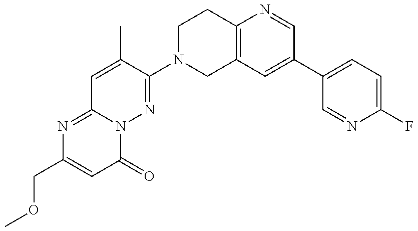 [SMILES: COCc1cc(=O)n2nc(N3CCc4ncc(-c5ccc(F)nc5)cc4C3)c(C)cc2n1] | 433.2 |
| 1-323 | 7-(3-(1,2-dimethyl-1H-imidazol-5- yl)-7,8-dihydro-1,6-naphthyridin- 6(5H)-yl)-8-methyl-4H- pyrimido[1,2-b]pyridazin-4-one | 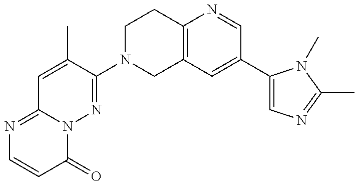 [SMILES: Cc1cc2nccc(=O)n2nc1N1CCc2ncc(-c3cnc(C)n3C)cc2C1] | 388.4 |
| 1-324 | 2-(methoxymethyl)-8-methyl-7-(3- (pyrimidin-5-yl)-7,8-dihydro- 1,6-naphthyridin- 6(5H)-yl)-4H-pyrimido[1,2-b] pyridazin-4-one | 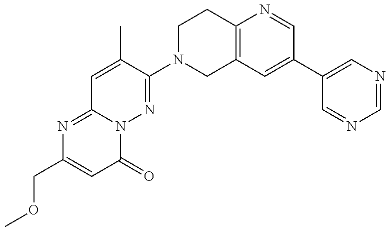 [SMILES: COCc1cc(=O)n2nc(N3CCc4ncc(-c5cncnc5)cc4C3)c(C)cc2n1] | 416.2 |
| 1-325 | 7-(3-(6,7-dihydro-4H-pyrazolo[5,1- c][1,4]oxazin-3-yl)-7,8-dihydro- 1,6-naphthyridin-6(5H)-yl)-8- methyl-4H-pyrimido[1,2- b]pyridazin-4-one | 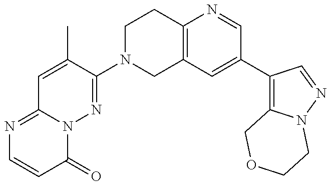 [SMILES: Cc1cc2nccc(=O)n2nc1N1CCc2ncc(-c3cnn4c3COCC4)cc2C1] | 416.3 |
| 1-326 | 7-(3-(5-chloro-6-fluoropyridin-3- yl)-7,8-dihydro-1,6-naphthyridin- 6(5H)-yl)-8-methyl-4H- pyrimido[1,2-b]pyridazin-4-one | 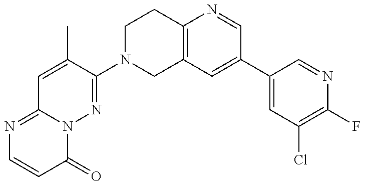 [SMILES: Cc1cc2nccc(=O)n2nc1N1CCc2ncc(-c3cnc(F)c(Cl)c3)cc2C1] | 423.3 |
| 1-327 | 7-(3-(6-fluoro-4-methylpyridin-3- yl)-7,8-dihydro-1,6-naphthyridin- 6(5H)-yl)-2-(methoxymethyl)-8- methyl-4H-pyrimido[1,2- b]pyridazin-4-one | 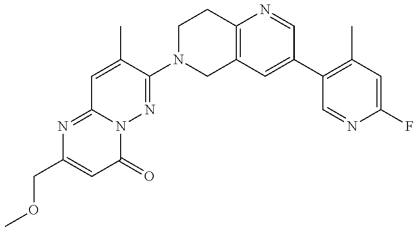 [SMILES: COCc1cc(=O)n2nc(N3CCc4ncc(-c5cnc(F)cc5C)cc4C3)c(C)cc2n1] | 447.2 |
| 1-328 | 7-(3-(2-fluoro-5-methylpyridin-4- yl)-7,8-dihydro-1,6-naphthyridin- 6(5H)-yl)-2-(methoxymethyl)-8- methyl-4H-pyrimido[1,2-b] pyridazin-4-one | 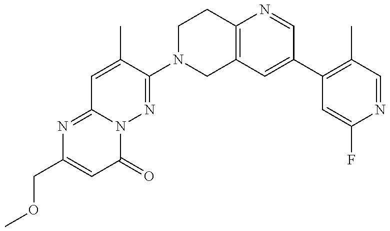 [SMILES: COCc1cc(=O)n2nc(N3CCc4ncc(-c5cc(F)ncc5C)cc4C3)c(C)cc2n1] | 447.2 |
| 1-329 | 7-(3-(6-chloro-4- (difluoromethyl)pyridin-3-yl)-7,8- dihydro-1,6-naphthyridin- 6(5H)-yl)-8-methyl-4H- pyrimido[1,2-b]pyridazin-4-one | 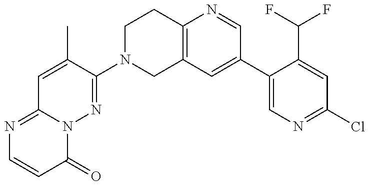 [SMILES: Cc1cc2nccc(=O)n2nc1N1CCc2ncc(-c3cnc(Cl)cc3C(F)F)cc2C1] | 455 |
| 1-330 | 7-(3-(3,4-difluorophenyl)-7,8- dihydro-1,6-naphthyridin- 6(5H)-yl)-2-(methoxymethyl)-8- methyl-4H-pyrimido[1,2-b] pyridazin-4-one | 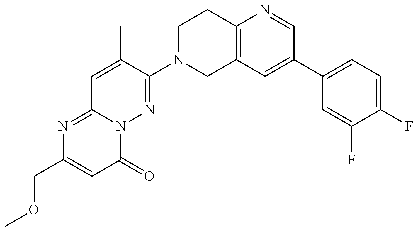 [SMILES: COCc1cc(=O)n2nc(N3CCc4ncc(-c5ccc(F)c(F)c5)cc4C3)c(C)cc2n1] | 450.2 |
| 1-331 | 7-(3-(5,6-difluoropyridin-3-yl)-7,8- dihydro-1,6-naphthyridin- 6(5H)-yl)-8-methyl-4H-pyrimido [1,2-b]pyridazin-4-one | 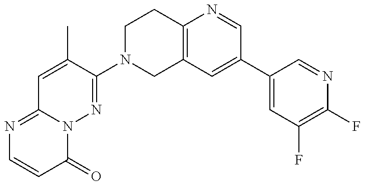 [SMILES: Cc1cc2nccc(=O)n2nc1N1CCc2ncc(-c3cnc(F)c(F)c3)cc2C1] | 407.2 |
| 1-332 | 7-(3-(5-fluoro-2-methylphenyl)-7,8- dihydro-1,6-naphthyridin- 6(5H)-yl)-2-(methoxymethyl)-8- methyl-4H-pyrimido[1,2-b] pyridazin-4-one | 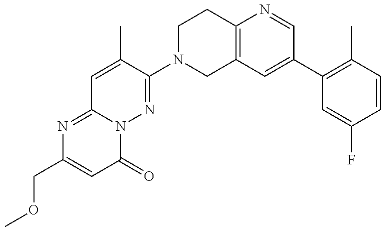 [SMILES: COCc1cc(=O)n2nc(N3CCc4ncc(-c5cc(F)ccc5C)cc4C3)c(C)cc2n1] | 446.2 |
| 1-333 | 7-(3-(6-chloro-4-methylpyridin-3- yl)-7,8-dihydro-1,6-naphthyridin- 6(5H)-yl)-8-methyl-4H- pyrimido[1,2-b]pyridazin-4-one | 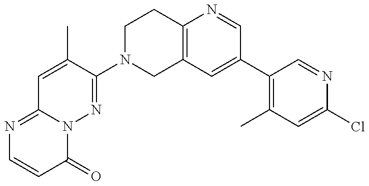 [SMILES: Cc1cc(Cl)ncc1-c1cnc2c(c1)CN(c1nn3c(=O)ccnc3cc1C)CC2] | 419.2 |
| 1-334 | 7-(3-(6-chloropyridazin-3-yl)-7,8- dihydro-1,6-naphthyridin- 6(5H)-yl)-8-methyl-4H-pyrimido [1,2-b]pyridazin-4-one | 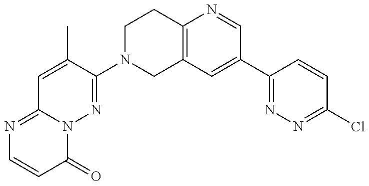 [SMILES: Cc1cc2nccc(=O)n2nc1N1CCc2ncc(-c3ccc(Cl)nn3)cc2C1] | 406.2 |
| 1-335 | 2-(methoxymethyl)-8-methyl-7-(3- (2,4,5-trifluorophenyl)-7,8-dihydro- 1,6-naphthyridin-6(5H)-yl)-4H- pyrimido[1,2-b]pyridazin-4-one | 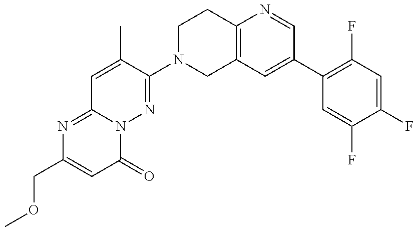 [SMILES: COCc1cc(=O)n2nc(N3CCc4ncc(-c5cc(F)c(F)cc5F)cc4C3)c(C)cc2n1] | 468.2 |
| 1-336 | 7-(3-(2,5-difluorophenyl)-7,8- dihydro-1,6-naphthyridin- 6(5H)-yl)-2-(methoxymethyl)-8- methyl-4H-pyrimido[1,2-b] pyridazin-4-one | 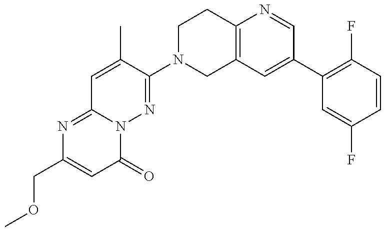 [SMILES: COCc1cc(=O)n2nc(N3CCc4ncc(-c5cc(F)ccc5F)cc4C3)c(C)cc2n1] | 450.2 |
| 1-337 | 2-(methoxymethyl)-8-methyl-7-(3- (o-tolyl)-7,8-dihydro-1,6- naphthyridin-6(5H)-yl)-4H- pyrimido[1,2-b]pyridazin-4-one | 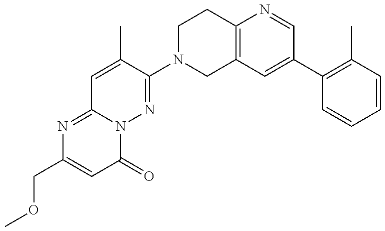 [SMILES: COCc1cc(=O)n2nc(N3CCc4ncc(-c5ccccc5C)cc4C3)c(C)cc2n1] | 428.2 |
| 1-338 | 7-(3-(1,5-dimethyl-1H-pyrazol-4- yl)-7,8-dihydro-1,6-naphthyridin- 6(5H)-yl)-8,9-dimethyl-4H- pyrimido[1,2-b]pyridazin-4-one | 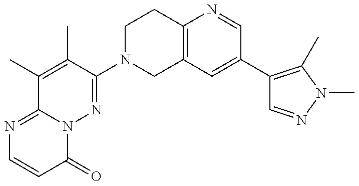 [SMILES: Cc1c(N2CCc3ncc(-c4cnn(C)c4C)cc3C2)nn2c(=O)ccnc2c1C] | 402.4 |
| 1-339 | 7-(3-(1-(difluoromethyl)-1H- pyrazol-4-yl)-7,8-dihydro- 1,6-naphthyridin- 6(5H)-yl)-8,9-dimethyl-4H- pyrimido[1,2-b]pyridazin-4-one | 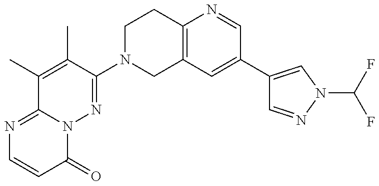 [SMILES: Cc1c(N2CCc3ncc(-c4cnn(C(F)F)c4)cc3C2)nn2c(=O)ccnc2c1C] | 424.4 |
| 1-340 | 7-(3-(2-isopropylphenyl)-7,8- dihydro-1,6-naphthyridin- 6(5H)-yl)-8,9-dimethyl-4H- pyrimido[1,2-b]pyridazin-4-one | 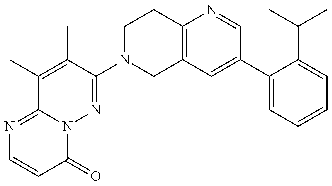 [SMILES: Cc1c(N2CCc3ncc(-c4ccccc4C(C)C)cc3C2)nn2c(=O)ccnc2c1C] | 426.3 |
| 1-341 | 7-(3-(2,3-difluoro-5-methylphenyl)- 7,8-dihydro-1,6-naphthyridin- 6(5H)-yl)-8,9-dimethyl-4H- pyrimido[1,2-b]pyridazin-4-one | 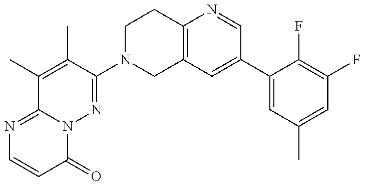 [SMILES: Cc1cc(F)c(F)c(-c2cnc3c(c2)CN(c2nn4c(=O)ccnc4c(C)c2C)CC3)c1] | 434.3 |
| 1-342 | 7-(3-(2-ethylphenyl)-7,8-dihydro- 1,6-naphthyridin- 6(5H)-yl)-8,9-dimethyl-4H- pyrimido[1,2-b]pyridazin-4-one | 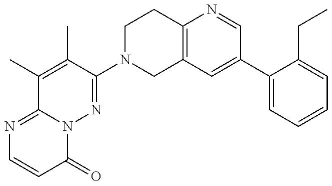 [SMILES: CCc1ccccc1-c1cnc2c(c1)CN(c1nn3c(=O)ccnc3c(C)c1C)CC2] | 412.4 |
| 1-343 | 7-(3-(2-fluorophenyl)-7,8-dihydro- 1,6-naphthyridin-6(5H)-yl)-8,9- dimethyl-4H-pyrimido[1,2- b]pyridazin-4-one | 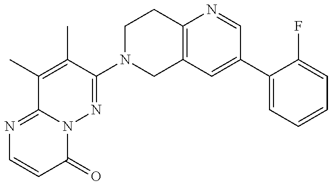 [SMILES: Cc1c(N2CCc3ncc(-c4ccccc4F)cc3C2)nn2c(=O)ccnc2c1C] | 402.2 |
| 1-344 | 7-(3-(2-methoxyphenyl)-7,8- dihydro-1,6-naphthyridin- 6(5H)-yl)-8,9-dimethyl-4H- pyrimido[1,2-b]pyridazin-4-one | 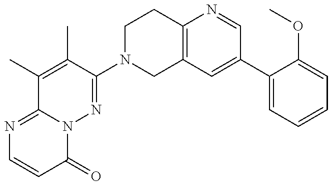 [SMILES: COc1ccccc1-c1cnc2c(c1)CN(c1nn3c(=O)ccnc3c(C)c1C)CC2] | 414.2 |
| 1-345 | 7-(3-(2,5-difluorophenyl)-7,8- dihydro-1,6-naphthyridin- 6(5H)-yl)-8,9-dimethyl-4H- pyrimido[1,2-b]pyridazin-4-one | 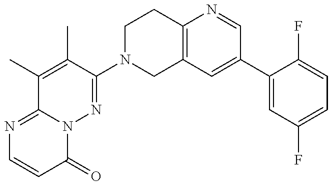 [SMILES: Cc1c(N2CCc3ncc(-c4cc(F)ccc4F)cc3C2)nn2c(=O)ccnc2c1C] | 420.2 |
| 1-346 | 8,9-dimethyl-7-(3-(2,3,5- trifluorophenyl)-7,8-dihydro-1,6- naphthyridin-6(5H)-yl)-4H- pyrimido[1,2-b]pyridazin-4-one | 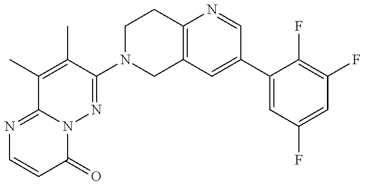 [SMILES: Cc1c(N2CCc3ncc(-c4cc(F)cc(F)c4F)cc3C2)nn2c(=O)ccnc2c1C] | 438.2 |
| 1-347 | 7-(3-(2,4-difluorophenyl)-7,8- dihydro-1,6-naphthyridin- 6(5H)-yl)-8,9-dimethyl-4H- pyrimido[1,2-b]pyridazin-4-one | 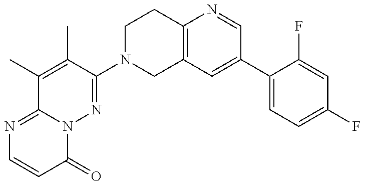 [SMILES: Cc1c(N2CCc3ncc(-c4ccc(F)cc4F)cc3C2)nn2c(=O)ccnc2c1C] | 420.2 |
| 1-348 | 8-methyl-7-(3-(3-methylpyridin-2- yl)-7,8-dihydro-1,6-naphthyridin- 6(5H)-yl)-4H-pyrimido[1,2- b]pyridazin-4-one | 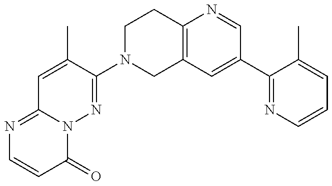 [SMILES: Cc1cccnc1-c1cnc2c(c1)CN(c1nn3c(=O)ccnc3cc1C)CC2] | 385.2 |
| 1-349 | 8-methyl-7-(3-(pyridin-2-yl)-7,8- dihydro-1,6-naphthyridin- 6(5H)-yl)-4H-pyrimido[1,2-b] pyridazin-4-one | 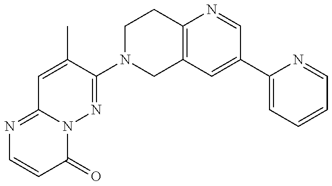 [SMILES: Cc1cc2nccc(=O)n2nc1N1CCc2ncc(-c3ccccn3)cc2C1] | 371.2 |
| 1-350 | 7-(3-(5-fluoro-3-methylpyridin-2- yl)-7,8-dihydro-1,6-naphthyridin- 6(5H)-yl)-8-methyl-4H- pyrimido[1,2-b]pyridazin-4-one | 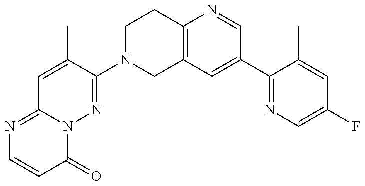 [SMILES: Cc1cc(F)cnc1-c1cnc2c(c1)CN(c1nn3c(=O)ccnc3cc1C)CC2] | 403.2 |
| 1-351 | 8-methyl-7-(3-(5-methylpyridin-2- yl)-7,8-dihydro-1,6-naphthyridin- 6(5H)-yl)-4H-pyrimido[1,2- b]pyridazin-4-one | 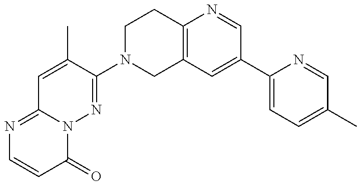 [SMILES: Cc1ccc(-c2cnc3c(c2)CN(c2nn4c(=O)ccnc4cc2C)CC3)nc1] | 385.2 |
| 1-352 | 8-methyl-7-(3-(pyridazin-3-yl)-7,8- dihydro-1,6-naphthyridin-6(5H)- yl)-4H-pyrimido[1,2-b]pyridazin-4- one | 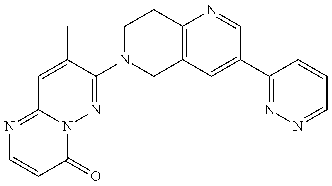 [SMILES: Cc1cc2nccc(=O)n2nc1N1CCc2ncc(-c3cccnn3)cc2C1] | 372.2 |
| 1-353 | 8-methyl-7-(3-(6-methylpyridazin- 3-yl)-7,8-dihydro-1,6-naphthyridin- 6(5H)-yl)-4H-pyrimido[1,2-b] pyridazin-4-one | 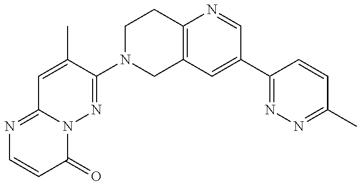 [SMILES: Cc1ccc(-c2cnc3c(c2)CN(c2nn4c(=O)ccnc4cc2C)CC3)nn1] | 386.2 |
| 1-354 | 7-(3-(4-fluoropyridin-2-yl)-7,8- dihydro-1,6-naphthyridin- 6(5H)-yl)-8-methyl-4H-pyrimido [1,2-b]pyridazin-4-one | 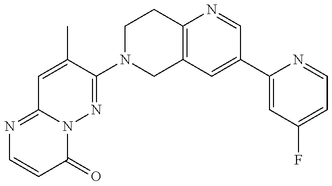 [SMILES: Cc1cc2nccc(=O)n2nc1N1CCc2ncc(-c3cc(F)ccn3)cc2C1] | 389.3 |
| 1-355 | 4-methyl-5-(6-(8-methyl-4-oxo-4H- pyrimido[1,2-b]pyridazin-7-yl)- 5,6,7,8-tetrahydro-1,6-naphthyridin- 3-yl)picolinonitrile | 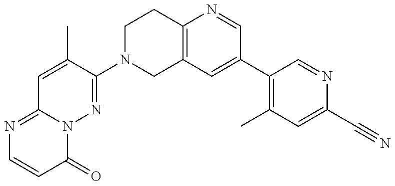 [SMILES: Cc1cc(C#N)ncc1-c1cnc2c(c1)CN(c1nn3c(=O)ccnc3cc1C)CC2] | 410.2 |
| 1-356 | 8-methyl-7-(3-(4-methylpyridazin- 3-yl)-7,8-dihydro-1,6-naphthyridin- 6(5H)-yl)-4H-pyrimido[1,2- b]pyridazin-4-one | 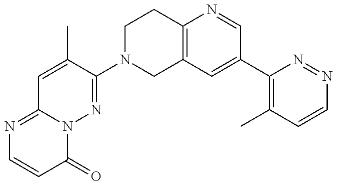 [SMILES: Cc1ccnnc1-c1cnc2c(c1)CN(c1nn3c(=O)ccnc3cc1C)CC2] | 386.3 |
| 1-357 | 7-(3-(6-chloro-4-methylpyridin-3- yl)-7,8-dihydro-1,6-naphthyridin- 6(5H)-yl)-2-(methoxymethyl)-8- methyl-4H-pyrimido[1,2- b]pyridazin-4-one | 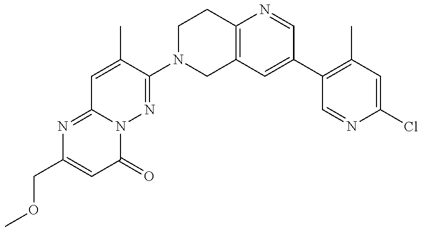 [SMILES: COCc1cc(=O)n2nc(N3CCc4ncc(-c5cnc(Cl)cc5C)cc4C3)c(C)cc2n1] | 463.3 |
| 1-358 | 7-(3-(3-fluorophenyl)-7,8-dihydro- 1,6-naphthyridin- 6(5H)-yl)-2-(methoxymethyl)-8- methyl-4H-pyrimido[1,2-b] pyridazin-4-one | 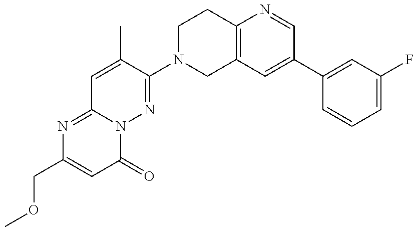 [SMILES: COCc1cc(=O)n2nc(N3CCc4ncc(-c5cccc(F)c5)cc4C3)c(C)cc2n1] | 432.4 |
| 1-359 | 2-(methoxymethyl)-8-methyl-7-(3- (4-methylpyridin-3-yl)-7,8-dihydro- 1,6-naphthyridin- 6(5H)-yl)-4H-pyrimido[1,2-b] pyridazin-4-one | 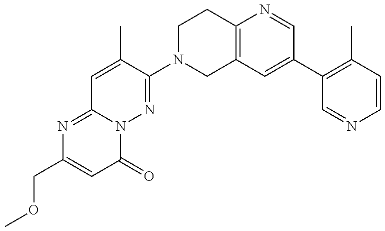 [SMILES: COCc1cc(=O)n2nc(N3CCc4ncc(-c5cnccc5C)cc4C3)c(C)cc2n1] | 429.4 |
| 1-360 | 7-(3-(2-fluorophenyl)-7,8-dihydro- 1,6-naphthyridin- 6(5H)-yl)-2-(methoxymethyl)-8- methyl-4H-pyrimido[1,2-b] pyridazin-4-one | 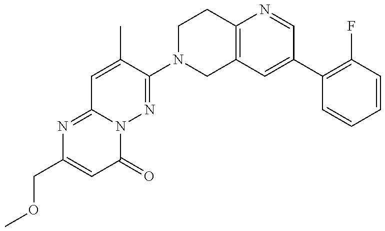 [SMILES: COCc1cc(=O)n2nc(N3CCc4ncc(-c5ccccc5F)cc4C3)c(C)cc2n1] | 432.4 |
| 1-361 | 2-(methoxymethyl)-8-methyl-7-(3- (2-methylpyridin-4-yl)-7,8-dihydro- 1,6-naphthyridin- 6(5H)-yl)-4H-pyrimido[1,2-b] pyridazin-4-one | 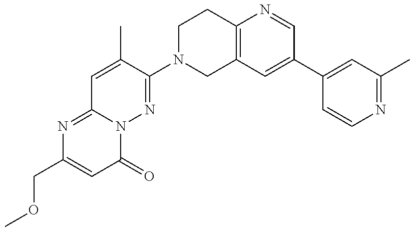 | 429.4 |
| 1-362 | 2-(methoxymethyl)-8-methyl-7-(3- (pyridin-4-yl)-7,8-dihydro- 1,6-naphthyridin- 6(5H)-yl)-4H-pyrimido[1,2-b] pyridazin-4-one | 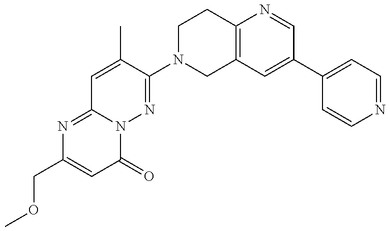 [SMILES: COCc1cc(=O)n2nc(N3CCc4ncc(-c5ccncc5)cc4C3)c(C)cc2n1] | 415.4 |
| 1-363 | 2-(methoxymethyl)-8-methyl-7-(3- (2-(trifluoromethoxy)phenyl)-7,8- dihydro-1,6-naphthyridin- 6(5H)-yl)-4H-pyrimido[1,2-b] pyridazin-4-one | 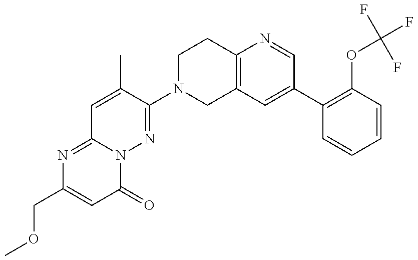 [SMILES: COCc1cc(=O)n2nc(N3CCc4ncc(-c5ccccc5OC(F)(F)F)cc4C3)c(C)cc2n1] | 498.4 |
| 1-364 | 7-(3-(1-cyclopropyl-1H-pyrazol-4- yl)-7,8-dihydro-1,6-naphthyridin- 6(5H)-yl)-2-(methoxymethyl)-8- methyl-4H-pyrimido[1,2- b]pyridazin-4-one | 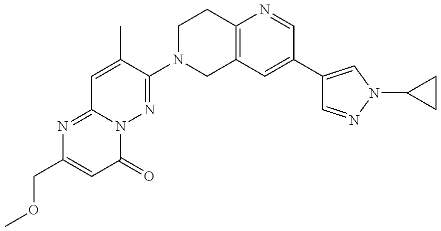 [SMILES: COCc1cc(=O)n2nc(N3CCc4ncc(-c5cnn(C6CC6)c5)cc4C3)c(C)cc2n1] | 444.4 |
| 1-365 | 2-(methoxymethyl)-8-methyl-7-(3- (pyridin-3-yl)-7,8-dihydro- 1,6-naphthyridin- 6(5H)-yl)-4H-pyrimido[1,2-b] pyridazin-4-one | 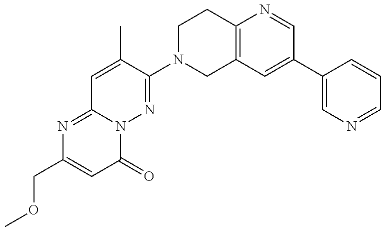 [SMILES: COCc1cc(=O)n2nc(N3CCc4ncc(-c5cccnc5)cc4C3)c(C)cc2n1] | 415.4 |
| 1-366 | 7-(3-(1,5-dimethyl-1H-pyrazol-4- yl)-7,8-dihydro-1,6-naphthyridin- 6(5H)-yl)-2-(methoxymethyl)-8- methyl-4H-pyrimido[1,2- b]pyridazin-4-one | 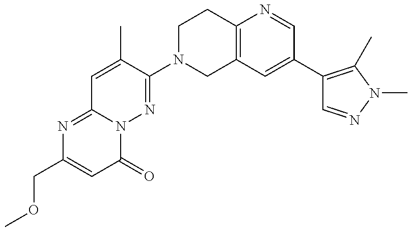 | 432.4 |
| 1-367 | 2-(methoxymethyl)-8-methyl-7-(3- (thiophen-2-yl)-7,8-dihydro- 1,6-naphthyridin- 6(5H)-yl)-4H-pyrimido[1,2-b] pyridazin-4-one | 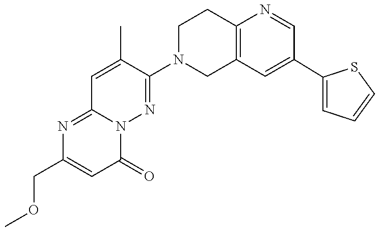 [SMILES: COCc1cc(=O)n2nc(N3CCc4ncc(-c5cccs5)cc4C3)c(C)cc2n1] | 420.3 |
| 1-368 | 2-(methoxymethyl)-7-(3-(3- methoxythiophen-2-yl)-7,8- dihydro-1,6-naphthyridin- 6(5H)-yl)-8-methyl-4H- pyrimido[1,2-b]pyridazin-4-one | 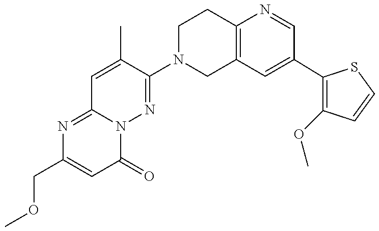 [SMILES: COCc1cc(=O)n2nc(N3CCc4ncc(-c5sccc5OC)cc4C3)c(C)cc2n1] | 450.3 |
| 1-369 | 7-(3-(5-(hydroxymethyl)thiophen- 2-yl)-7,8-dihydro-1,6-naphthyridin- 6(5H)-yl)-2-(methoxymethyl)-8- methyl-4H-pyrimido[1,2-b] pyridazin-4-one | 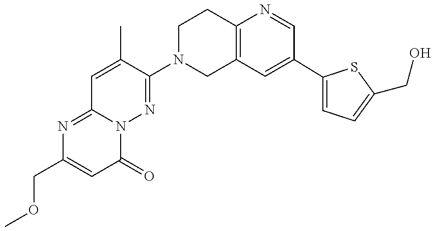 [SMILES: COCc1cc(=O)n2nc(N3CCc4ncc(-c5ccc(CO)s5)cc4C3)c(C)cc2n1] | 450.3 |
| 1-370 | 2-(methoxymethyl)-8-methyl-7-(3- (thiophen-3-yl)-7,8-dihydro-1,6- naphthyridin-6(5H)-yl)-4H- pyrimido[1,2-b]pyridazin-4-one | 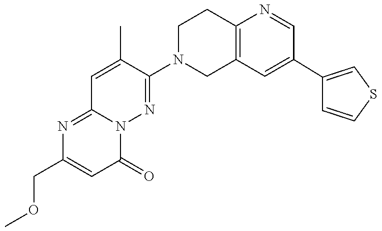 [SMILES: COCc1cc(=O)n2nc(N3CCc4ncc(-c5ccsc5)cc4C3)c(C)cc2n1] | 420.4 |
| 1-371 | 7-(3-(furan-2-yl)-7,8-dihydro- 1,6-naphthyridin-6(5H)-yl)-2- (methoxymethyl)-8-methyl-4H- pyrimido[1,2-b]pyridazin-4-one | 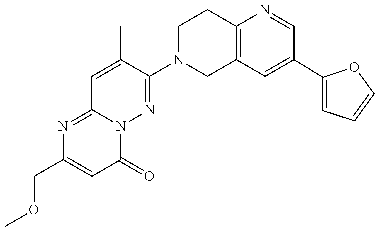 [SMILES: COCc1cc(=O)n2nc(N3CCc4ncc(-c5ccco5)cc4C3)c(C)cc2n1] | 404.4 |
| 1-372 | 2-(methoxymethyl)-8-methyl-7-(3- (5-methylfuran-2-yl)-7,8-dihydro- 1,6-naphthyridin- 6(5H)-yl)-4H-pyrimido[1,2-b] pyridazin-4-one | 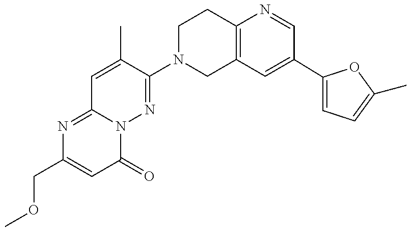 | 418.4 |
| 1-373 | 7-(3-(6-(difluoromethoxy)pyridin- 3-yl)-7,8-dihydro-1,6-naphthyridin- 6(5H)-yl)-2-(methoxymethyl)-8,9- dimethyl-4H-pyrimido[1,2- b]pyridazin-4-one | 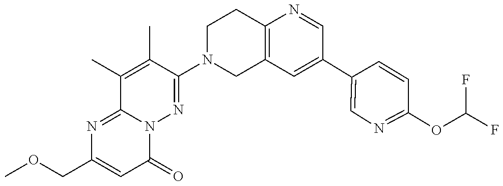 [SMILES: COCc1cc(=O)n2nc(N3CCc4ncc(-c5ccc(OC(F)F)nc5)cc4C3)c(C)c(C)c2n1] | 495.2 |
| 1-374 | 7-(3-(6-(difluoromethoxy)pyridin- 3-yl)-7,8-dihydro-1,6-naphthyridin- 6(5H)-yl)-2-(methoxymethyl)-8- methyl-4H-pyrimido[1,2- b]pyridazin-4-one | 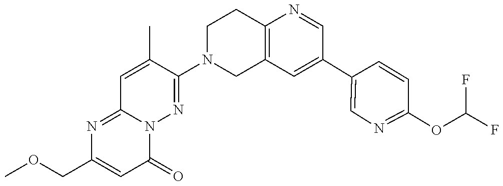 [SMILES: COCc1cc(=O)n2nc(N3CCc4ncc(-c5ccc(OC(F)F)nc5)cc4C3)c(C)cc2n1] | 481.2 |
| 1-375 | 7-(3-(6-(difluoromethyl)-4- methylpyridin-3-yl)-7,8-dihydro- 1,6-naphthyridin- 6(5H)-yl)-8-methyl-4H-pyrimido [1,2-b]pyridazin-4-one | 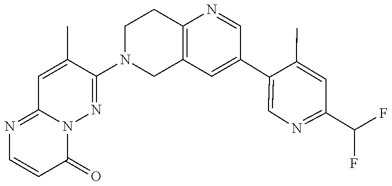 [SMILES: Cc1cc(C(F)F)ncc1-c1cnc2c(c1)CN(c1nn3c(=O)ccnc3cc1C)CC2] | 435.3 |
| 1-376^{a} | 2-(methoxymethyl)-8,9-dimethyl-7- (8-methyl-3-(trifluoromethyl)-7,8- dihydro-1,6-naphthyridin- 6(5H)-yl)-4H-pyrimido[1,2-b] pyridazin-4-one | 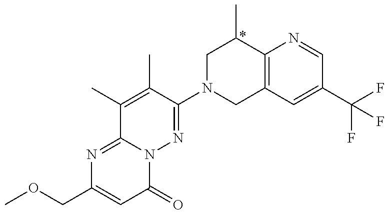 [SMILES: COCc1cc(=O)n2nc(N3Cc4cc(C(F)(F)F)cnc4C(C)C3)c(C)c(C)c2n1] | 434.2 |
| 1-377^{b} | 2-(methoxymethyl)-8,9-dimethyl-7- (8-methyl-3-(trifluoromethyl)-7,8- dihydro-1,6-naphthyridin- 6(5H)-yl)-4H-pyrimido[1,2-b] pyridazin-4-one | 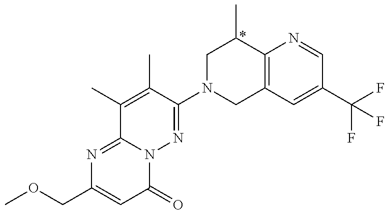 [SMILES: COCc1cc(=O)n2nc(N3Cc4cc(C(F)(F)F)cnc4C(C)C3)c(C)c(C)c2n1] | 434.4 |
| 1-378 | 8-methyl-7-(3-(pyridin-3-ylmethyl)- 7,8-dihydro-1,6-naphthyridin- 6(5H)-yl)-4H-pyrimido[1,2- b]pyridazin-4-one | 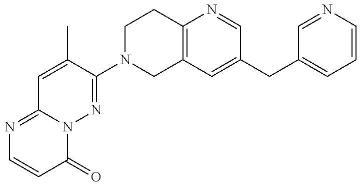 [SMILES: Cc1cc2nccc(=O)n2nc1N1CCc2ncc(Cc3cccnc3)cc2C1] | 385.2 |
| 1-379 | 7-(3-(6-chloro-4-methylpyridin-3- yl)-7,8-dihydro-1,6-naphthyridin- 6(5H)-yl)-2-(methoxymethyl)-8,9- dimethyl-4H-pyrimido[1,2- b]pyridazin-4-one | 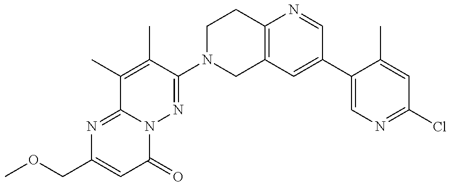 [SMILES: COCc1cc(=O)n2nc(N3CCc4ncc(-c5cnc(Cl)cc5C)cc4C3)c(C)c(C)c2n1] | 477.3 |
| 1-380 | 7-(3-(6,7-dihydro-4H-pyrazolo[5,1- c][1,4]oxazin-3-yl)-7,8-dihydro- 1,6-naphthyridin- 6(5H)-yl)-2-(methoxymethyl)- 8,9-dimethyl-4H-pyrimido[1,2-b] pyridazin-4-one | 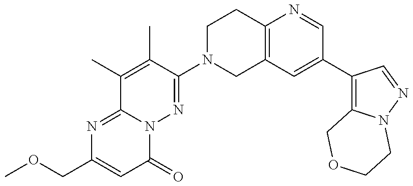 [SMILES: COCc1cc(=O)n2nc(N3CCc4ncc(-c5cnn6c5COCC6)cc4C3)c(C)c(C)c2n1] | 474.3 |
| 1-381 | 7-(3-(3,5-dimethylpyridin-2-yl)-7,8- dihydro-1,6-naphthyridin- 6(5H)-yl)-2-(methoxymethyl)-8- methyl-4H-pyrimido[1,2-b] pyridazin-4-one | 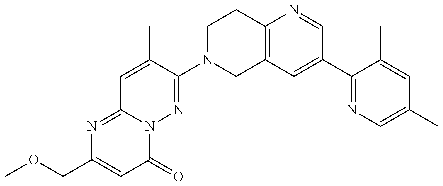 | 443.4 |
| 1-382 | 7-(3-(3,5-difluoropyridin-2-yl)-7,8- dihydro-1,6-naphthyridin- 6(5H)-yl)-2-(methoxymethyl)-8- methyl-4H-pyrimido[1,2-b] pyridazin-4-one | 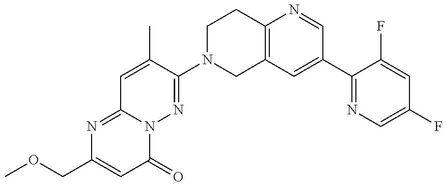 [SMILES: COCc1cc(=O)n2nc(N3CCc4ncc(-c5ncc(F)cc5F)cc4C3)c(C)cc2n1] | 451.2 |
| 1-383 | 7-(3-(4-fluoropyridin-2-yl)-7,8- dihydro-1,6-naphthyridin- 6(5H)-yl)-2-(methoxymethyl)-8- methyl-4H-pyrimido[1,2-b] pyridazin-4-one | 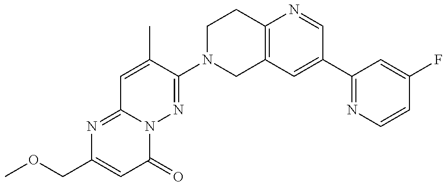 [SMILES: COCc1cc(=O)n2nc(N3CCc4ncc(-c5cc(F)ccn5)cc4C3)c(C)cc2n1] | 433.2 |
| 1-384 | 2-(methoxymethyl)-7-(3-(4- methoxypyridin-3-yl)-7,8-dihydro- 1,6-naphthyridin- 6(5H)-yl)-8-methyl-4H-pyrimido [1,2-b]pyridazin-4-one | 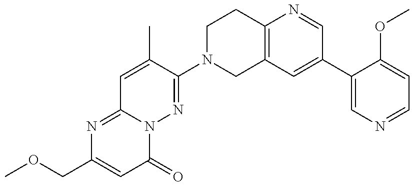 | 445.3 |
| 1-385^{b} | 8,9-dimethyl-7-(5-methyl-3- (trifluoromethyl)-7,8-dihydro- 1,6-naphthyridin- 6(5H)-yl)-4H-pyrimido[1,2-b] pyridazin-4-one | 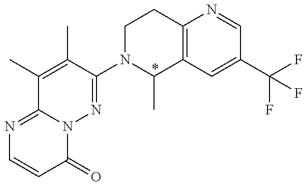 [SMILES: Cc1c(N2CCc3ncc(C(F)(F)F)cc3C2C)nn2c(=O)ccnc2c1C] | 390.2 |
| 1-386^{a} | 8,9-dimethyl-7-(5-methyl-3- (trifluoromethyl)-7,8-dihydro- 1,6-naphthyridin- 6(5H)-yl)-4H-pyrimido[1,2-b] pyridazin-4-one | 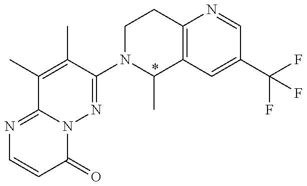 [SMILES: Cc1c(N2CCc3ncc(C(F)(F)F)cc3C2C)nn2c(=O)ccnc2c1C] | 390.2 |
| 1-387 | 7-(3-(6-methoxy-4-methylpyridin- 3-yl)-7,8-dihydro-1,6- naphthyridin-6(5H)-yl)-8- methyl-4H-pyrimido[1,2-b] pyridazin-4-one | 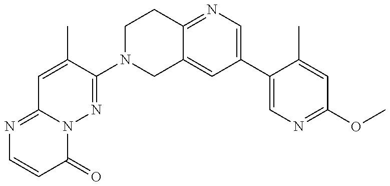 [SMILES: COc1cc(C)c(-c2cnc3c(c2)CN(c2nn4c(=O)ccnc4cc2C)CC3)cn1] | 415 |
| 1-388 | 7-(3-(1-acetyl-1,2,3,6- tetrahydropyridin-4-yl)-7,8- dihydro-1,6-naphthyridin- 6(5H)-yl)-8-methyl-4H- pyrimido[1,2-b]pyridazin-4-one | 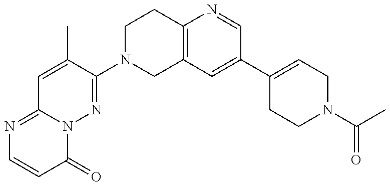 [SMILES: CC(=O)N1CC=C(c2cnc3c(c2)CN(c2nn4c(=O)ccnc4cc2C)CC3)CC1] | 417 |
| 1-389 | 8-methyl-7-(3-(2-methylthiazol-4- yl)-7,8-dihydro-1,6-naphthyridin- 6(5H)-yl)-4H-pyrimido[1,2-b] pyridazin-4-one | 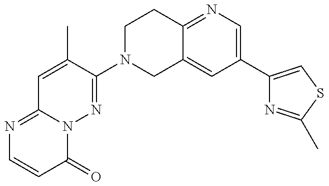 [SMILES: Cc1nc(-c2cnc3c(c2)CN(c2nn4c(=O)ccnc4cc2C)CC3)cs1] | 391 |
| 1-390 | 7-(3-(1,3-dimethyl-1H-pyrazol-5- yl)-7,8-dihydro-1,6-naphthyridin- 6(5H)-yl)-8-methyl-4H- pyrimido[1,2-b]pyridazin-4-one | 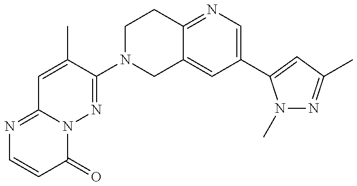 [SMILES: Cc1cc(-c2cnc3c(c2)CN(c2nn4c(=O)ccnc4cc2C)CC3)n(C)n1] | 388 |
| 1-391 | 7-(3-(1,5-dimethyl-1H-imidazol-2- yl)-7,8-dihydro-1,6-naphthyridin- 6(5H)-yl)-8-methyl-4H- pyrimido[1,2-b]pyridazin-4-one | 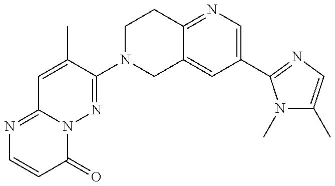 [SMILES: Cc1cc2nccc(=O)n2nc1N1CCc2ncc(-c3ncc(C)n3C)cc2C1] | 388 |
| 1-392 | 8-methyl-7-(3-(1-methyl-1H- imidazol-5-yl)-7,8-dihydro- 1,6-naphthyridin- 6(5H)-yl)-4H-pyrimido[1,2-b] pyridazin-4-one | 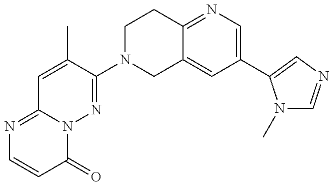 [SMILES: Cc1cc2nccc(=O)n2nc1N1CCc2ncc(-c3cncn3C)cc2C1] | 374 |
| 1-393 | 8-methyl-7-(3-(5-methylthiazol-4- yl)-7,8-dihydro-1,6-naphthyridin- 6(5H)-yl)-4H-pyrimido[1,2- b]pyridazin-4-one | 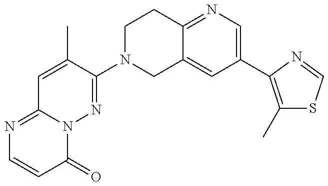 [SMILES: Cc1cc2nccc(=O)n2nc1N1CCc2ncc(-c3ncsc3C)cc2C1] | 391 |
| 1-394 | 8-methyl-7-(3-(4-methylthiophen-3- yl)-7,8-dihydro-1,6-naphthyridin- 6(5H)-yl)-4H-pyrimido[1,2- b]pyridazin-4-one | 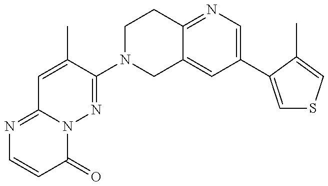 [SMILES: Cc1cscc1-c1cnc2c(c1)CN(c1nn3c(=O)ccnc3cc1C)CC2] | 390 |
| 1-395 | 8-methyl-7-(3-(4-methylthiazol-5- yl)-7,8-dihydro-1,6-naphthyridin- 6(5H)-yl)-4H-pyrimido[1,2- b]pyridazin-4-one | 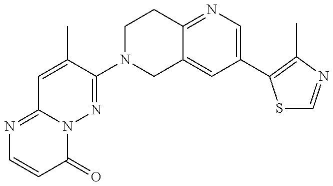 [SMILES: Cc1cc2nccc(=O)n2nc1N1CCc2ncc(-c3scnc3C)cc2C1] | 391 |
| 1-396 | 7-(3-(1-isopropyl-1H-pyrazol-5-yl)- 7,8-dihydro-1,6-naphthyridin- 6(5H)-yl)-8-methyl-4H- pyrimido[1,2-b]pyridazin-4-one | 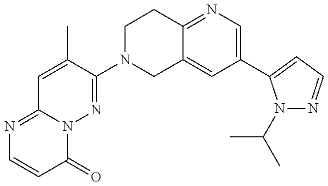 [SMILES: Cc1cc2nccc(=O)n2nc1N1CCc2ncc(-c3ccnn3C(C)C)cc2C1] | 402 |
| 1-397 | 8-methyl-7-(3-(2-methyloxazol-5- yl)-7,8-dihydro-1,6-naphthyridin- 6(5H)-yl)-4H-pyrimido[1,2- b]pyridazin-4-one | 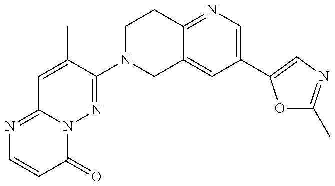 [SMILES: Cc1ncc(-c2cnc3c(c2)CN(c2nn4c(=O)ccnc4cc2C)CC3)o1] | 375 |
| 1-398 | 7-(3-(6-fluoro-4-methylpyridin-3- yl)-7,8-dihydro-1,6-naphthyridin- 6(5H)-yl)-8-methyl-4H- pyrimido[1,2-b]pyridazin-4-one | 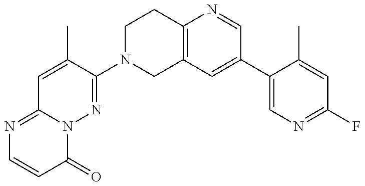 [SMILES: Cc1cc(F)ncc1-c1cnc2c(c1)CN(c1nn3c(=O)ccnc3cc1C)CC2] | 403 |
| 1-399 | 7-(3-(5-fluoro-4-methylpyridin-3- yl)-7,8-dihydro-1,6-naphthyridin- 6(5H)-yl)-8-methyl-4H- pyrimido[1,2-b]pyridazin-4-one | 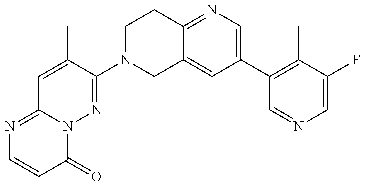 [SMILES: Cc1cc2nccc(=O)n2nc1N1CCc2ncc(-c3cncc(F)c3C)cc2C1] | 403 |
| 1-400 | 8-methyl-7-(3-(4-methyl-6- (trifluoromethyl)pyridin-3-yl)-7,8- dihydro-1,6-naphthyridin- 6(5H)-yl)-4H-pyrimido[1,2-b] pyridazin-4-one | 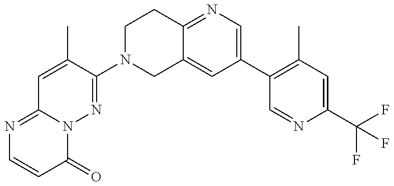 [SMILES: Cc1cc(C(F)(F)F)ncc1-c1cnc2c(c1)CN(c1nn3c(=O)ccnc3cc1C)CC2] | 453 |
| 1-401 | 7-(3-(2,4-dimethylpyridin-3-yl)-7,8- dihydro-1,6-naphthyridin- 6(5H)-yl)-8-methyl-4H-pyrimido [1,2-b]pyridazin-4-one | 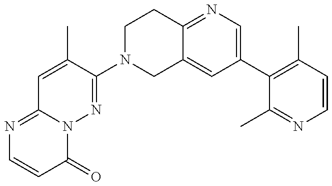 [SMILES: Cc1cc2nccc(=O)n2nc1N1CCc2ncc(-c3c(C)ccnc3C)cc2C1] | 399 |
| 1-402 | 7-(3-(4-acetylpyridin-3-yl)-7,8- dihydro-1,6-naphthyridin- 6(5H)-yl)-8-methyl-4H- pyrimido[1,2-b]pyridazin-4-one | 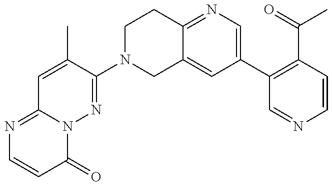 [SMILES: CC(=O)c1ccncc1-c1cnc2c(c1)CN(c1nn3c(=O)ccnc3cc1C)CC2] | 413 |
| 1-403 | 8-methyl-7-(3-(4-methylpyridin-3- yl)-7,8-dihydro-1,6-naphthyridin- 6(5H)-yl)-4H-pyrimido[1,2-b] pyridazin-4-one | 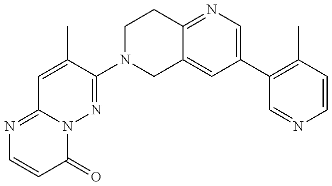 [SMILES: Cc1ccncc1-c1cnc2c(c1)CN(c1nn3c(=O)ccnc3cc1C)CC2] | 385 |
| 1-404 | 7-(3-(6-amino-4-methylpyridin-3- yl)-7,8-dihydro-1,6-naphthyridin- 6(5H)-yl)-8-methyl-4H- pyrimido[1,2-b]pyridazin-4-one | 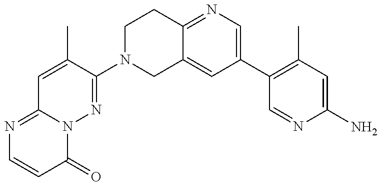 [SMILES: Cc1cc(N)ncc1-c1cnc2c(c1)CN(c1nn3c(=O)ccnc3cc1C)CC2] | 400 |
| 1-405 | 7-(3-(4,6-dimethylpyridin-3-yl)-7,8- dihydro-1,6-naphthyridin- 6(5H)-yl)-8-methyl-4H-pyrimido [1,2-b]pyridazin-4-one | 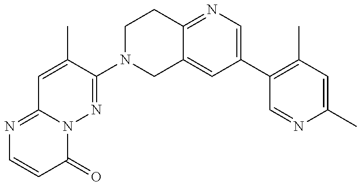 [SMILES: Cc1cc(C)c(-c2cnc3c(c2)CN(c2nn4c(=O)ccnc4cc2C)CC3)cn1] | 399 |
| 1-406 | 4-methyl-5-(6-(8-methyl-4-oxo-4H- pyrimido[1,2-b]pyridazin-7-yl)- 5,6,7,8-tetrahydro-1,6-naphthyridin- 3-yl)picolinic acid | 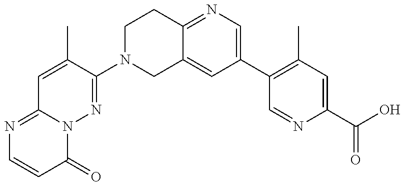 [SMILES: Cc1cc(C(=O)O)ncc1-c1cnc2c(c1)CN(c1nn3c(=O)ccnc3cc1C)CC2] | 429 |
| 1-407 | 7-(3-(6-(dimethylamino)-4- methylpyridin-3-yl)-7,8-dihydro- 1,6-naphthyridin- 6(5H)-yl)-8-methyl-4H-pyrimido [1,2-b]pyridazin-4-one | 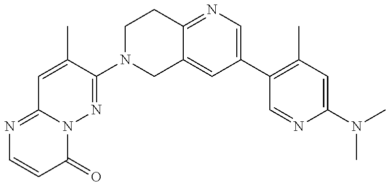 [SMILES: Cc1cc(N(C)C)ncc1-c1cnc2c(c1)CN(c1nn3c(=O)ccnc3cc1C)CC2] | 428 |
| 1-408 | 7-(3-(1,5-dimethyl-1H-pyrazol-4- yl)-7,8-dihydro-1,6-naphthyridin- 6(5H)-yl)-8-methyl-4H- pyrimido[1,2-b]pyridazin-4-one | 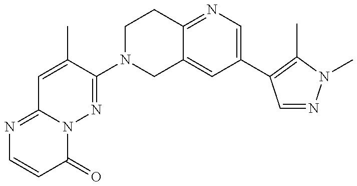 [SMILES: Cc1cc2nccc(=O)n2nc1N1CCc2ncc(-c3cnn(C)c3C)cc2C1] | 388 |
| 1-409 | 8-methyl-7-(3-(3-methylthiophen-2- yl)-7,8-dihydro-1,6-naphthyridin- 6(5H)-yl)-4H-pyrimido[1,2- b]pyridazin-4-one | 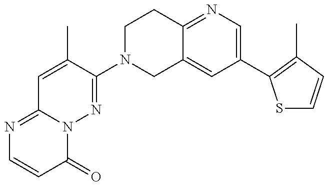 [SMILES: Cc1cc2nccc(=O)n2nc1N1CCc2ncc(-c3sccc3C)cc2C1] | 390 |
| 1-410 | 3-(6-(8-methyl-4-oxo-4H- pyrimido[1,2-b]pyridazin-7-yl)- 5,6,7,8-tetrahydro-1,6-naphthyridin- 3-yl)thiophene-2-carbonitrile | 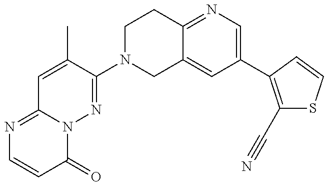 [SMILES: Cc1cc2nccc(=O)n2nc1N1CCc2ncc(-c3ccsc3C#N)cc2C1] | 401 |
| 1-411 | 8-methyl-7-(3-(1-methyl-3- (trifluoromethyl)-1H-pyrazol-4-yl)- 7,8-dihydro-1,6-naphthyridin- 6(5H)-yl)-4H-pyrimido[1,2- b]pyridazin-4-one | 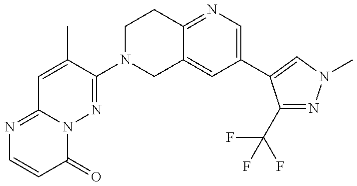 [SMILES: Cc1cc2nccc(=O)n2nc1N1CCc2ncc(-c3cn(C)nc3C(F)(F)F)cc2C1] | 442 |
| 1-412 | 7-(3-(3-(difluoromethyl)-1-methyl- 1H-pyrazol-4-yl)-7,8-dihydro- 1,6-naphthyridin- 6(5H)-yl)-8-methyl-4H-pyrimido [1,2-b]pyridazin-4-one | 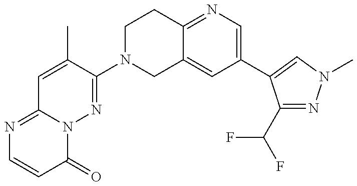 [SMILES: Cc1cc2nccc(=O)n2nc1N1CCc2ncc(-c3cn(C)nc3C(F)F)cc2C1] | 424 |
| 1-413 | 7-(3-(5-(difluoromethyl)-1-methyl- 1H-pyrazol-4-yl)-7,8-dihydro- 1,6-naphthyridin- 6(5H)-yl)-8-methyl-4H- pyrimido[1,2-b]pyridazin-4-one | 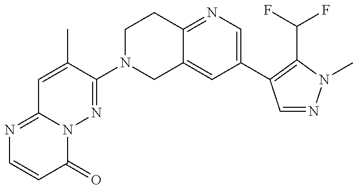 [SMILES: Cc1cc2nccc(=O)n2nc1N1CCc2ncc(-c3cnn(C)c3C(F)F)cc2C1] | 424 |
| 1-414 | 7-(3-(1-(difluoromethyl)-5-methyl- 1H-pyrazol-4-yl)-7,8-dihydro-1,6- naphthyridin-6(5H)-yl)-8-methyl- 4H-pyrimido[1,2-b]pyridazin-4-one | 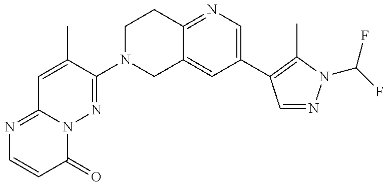 [SMILES: Cc1cc2nccc(=O)n2nc1N1CCc2ncc(-c3cnn(C(F)F)c3C)cc2C1] | 424 |
| 1-415 | 3-(6-(8-methyl-4-oxo-4H- pyrimido[1,2-b]pyridazin-7-yl)- 5,6,7,8-tetrahydro-1,6-naphthyridin- 3-yl)thiophene-2-carbaldehyde | 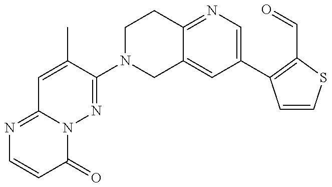 [SMILES: Cc1cc2nccc(=O)n2nc1N1CCc2ncc(-c3ccsc3C=O)cc2C1] | 404 |
| 1-416 | 7-(3-(3,5-dimethylisoxazol-4-yl)- 7,8-dihydro-1,6-naphthyridin- 6(5H)-yl)-8-methyl-4H- pyrimido[1,2-b]pyridazin-4-one | 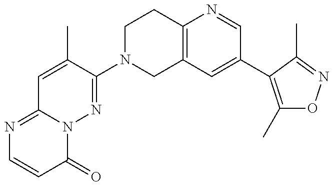 [SMILES: Cc1cc2nccc(=O)n2nc1N1CCc2ncc(-c3c(C)noc3C)cc2C1] | 389 |
| 1-417 | 7-(3-(1-(difluoromethyl)-3-methyl- 1H-pyrazol-4-yl)-7,8-dihydro-1,6- naphthyridin-6(5H)-yl)-8-methyl- 4H-pyrimido[1,2-b]pyridazin-4-one | 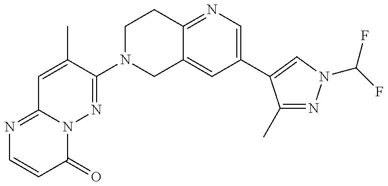 [SMILES: Cc1cc2nccc(=O)n2nc1N1CCc2ncc(-c3cn(C(F)F)nc3C)cc2C1] | 424 |
| 1-418 | 7-(3-(2-(2-hydroxypropan-2- yl)pyridin-3-yl)-7,8-dihydro-1,6- naphthyridin-6(5H)-yl)-8-methyl- 4H-pyrimido[1,2-b]pyridazin-4-one | 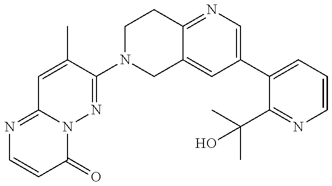 [SMILES: Cc1cc2nccc(=O)n2nc1N1CCc2ncc(-c3cccnc3C(C)(C)O)cc2C1] | 429 |
| 1-419 | 7-(3-(5-(2-hydroxypropan-2- yl)pyridin-3-yl)-7,8-dihydro-1,6- naphthyridin-6(5H)-yl)-8-methyl- 4H-pyrimido[1,2-b]pyridazin-4-one | 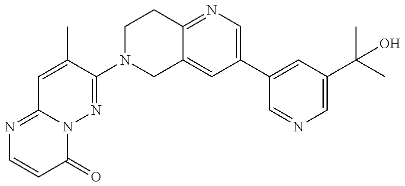 [SMILES: Cc1cc2nccc(=O)n2nc1N1CCc2ncc(-c3cncc(C(C)(C)O)c3)cc2C1] | 429 |
| 1-420 | 7-(3-(2,4-dimethylpyrimidin-5-yl)- 7,8-dihydro-1,6-naphthyridin- 6(5H)-yl)-8-methyl-4H- pyrimido[1,6-b]pyridazin-4-one | 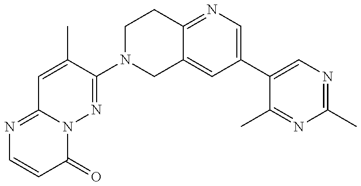 [SMILES: Cc1ncc(-c2cnc3c(c2)CN(c2nn4c(=O)ccnc4cc2C)CC3)c(C)n1] | 400 |
| 1-421 | 6-methyl-5-(6-(8-methyl-4-oxo-4H- pyrimido[1,2-b]pyridazin-7-yl)- 5,6,7,8-tetrahydro-1,6-naphthyridin- 3-yl)picolinonitrile | 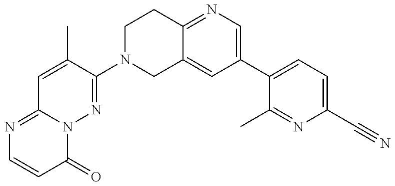 [SMILES: Cc1cc2nccc(=O)n2nc1N1CCc2ncc(-c3ccc(C#N)nc3C)cc2C1] | 410 |
| 1-422 | 7-(3-(2-isopropylpyridin-3-yl)-7,8- dihydro-1,6-naphthyridin- 6(5H)-yl)-8-methyl-4H-pyrimido [1,2-b]pyridazin-4-one | 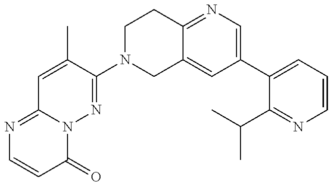 [SMILES: Cc1cc2nccc(=O)n2nc1N1CCc2ncc(-c3cccnc3C(C)C)cc2C1] | 413 |
| 1-423 | 1,5-dimethyl-4-(6-(8-methyl-4-oxo- 4H-pyrimido[1,2-b]pyridazin-7-yl)- 5,6,7,8-tetrahydro-1,6-naphthyridin- 3-yl)-1H-pyrrole-2-carbonitrile | 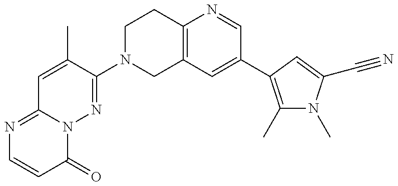 [SMILES: Cc1cc2nccc(=O)n2nc1N1CCc2ncc(-c3cc(C#N)n(C)c3C)cc2C1] | 412 |
| 1-424 | 8-methyl-7-(3-(1,3,5-trimethyl-1H- pyrazol-4-yl)-7,8-dihydro- 1,6-naphthyridin- 6(5H)-yl)-4H-pyrimido[1,2-b] pyridazin-4-one | 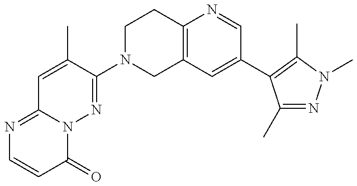 [SMILES: Cc1cc2nccc(=O)n2nc1N1CCc2ncc(-c3c(C)nn(C)c3C)cc2C1] | 402 |
| 1-425 | 7-(3-(2-(difluoromethyl)pyridin-3- yl)-7,8-dihydro-1,6-naphthyridin- 6(5H)-yl)-8-methyl-4H- pyrimido[1,2-b]pyridazin-4-one | 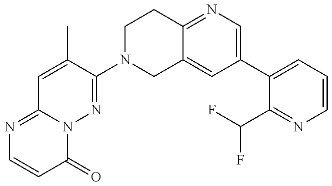 [SMILES: Cc1cc2nccc(=O)n2nc1N1CCc2ncc(-c3cccnc3C(F)F)cc2C1] | 421 |
| 1-426 | 7-(3-(6-methoxy-2-methylpyridin- 3-yl)-7,8-dihydro-1,6-naphthyridin- 6(5H)-yl)-8-methyl-4H- pyrimido[1,2-b]pyridazin-4-one | 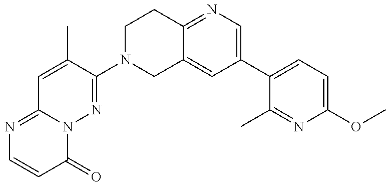 [SMILES: COc1ccc(-c2cnc3c(c2)CN(c2nn4c(=O)ccnc4cc2C)CC3)c(C)n1] | 415 |
| 1-427 | 7-(3-(2-methoxy-3-methylpyridin- 4-yl)-7,8-dihydro-1,6-naphthyridin- 6(5H)-yl)-8-methyl-4H- pyrimido[1,2-b]pyridazin-4-one | 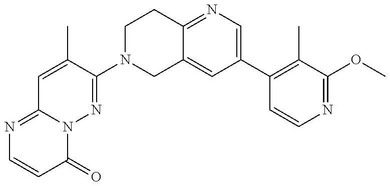 [SMILES: COc1nccc(-c2cnc3c(c2)CN(c2nn4c(=O)ccnc4cc2C)CC3)c1C] | 415 |
| 1-428 | N-cyclopropyl-4-methyl-5-(6-(8- methyl-4-oxo-4H-pyrimido[1,2- b]pyridazin-7-yl)-5,6,7,8- tetrahydro-1,6-naphthyridin-3- yl)thiophene-2-carboxamide | 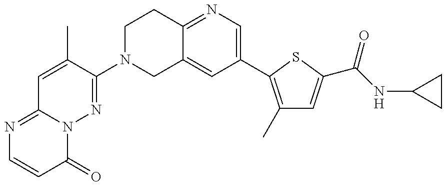 [SMILES: Cc1cc2nccc(=O)n2nc1N1CCc2ncc(-c3sc(C(=O)NC4CC4)cc3C)cc2C1] | 473 |
| 1-429 | 8,9-dimethyl-7-(3-(tetrahydro-2H- pyran-4-yl)-7,8-dihydro- 1,6-naphthyridin- 6(5H)-yl)-4H-pyrimido[1,2-b] pyridazin-4-one | 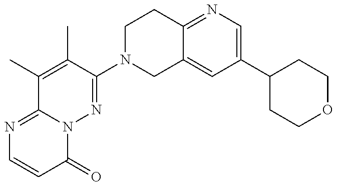 [SMILES: Cc1c(N2CCc3ncc(C4CCOCC4)cc3C2)nn2c(=O)ccnc2c1C] | 392.1 |
| 1-430 | 7-(3-cyclohexyl-7,8-dihydro-1,6- naphthyridin-6(5H)-yl)-8,9- dimethyl-4H-pyrimido[1,2-b] pyridazin-4-one | 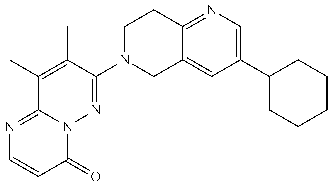 [SMILES: Cc1c(N2CCc3ncc(C4CCCCC4)cc3C2)nn2c(=O)ccnc2c1C] | 390.1 |
| 1-431 | 8-methyl-7-(3-(o-tolyl)-7,8- dihydro-1,6-naphthyridin- 6(5H)-yl)-4H-pyrimido[1,2-b] pyridazin-4-one | 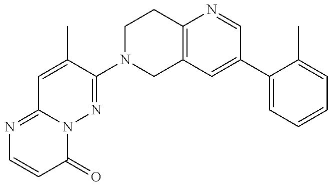 [SMILES: Cc1ccccc1-c1cnc2c(c1)CN(c1nn3c(=O)ccnc3cc1C)CC2] | 384 |
| 1-432 | 7-(3-(5-fluoro-2-methylphenyl)-7,8- dihydro-1,6-naphthyridin- 6(5H)-yl)-8-methyl-4H-pyrimido [1,2-b]pyridazin-4-one | 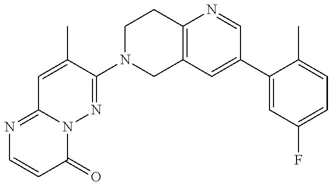 [SMILES: Cc1ccc(F)cc1-c1cnc2c(c1)CN(c1nn3c(=O)ccnc3cc1C)CC2] | 402 |
| 1-433 | 7-(3-(2-(1,1-difluoroethyl)phenyl)- 7,8-dihydro-1,6-naphthyridin- 6(5H)-yl)-8-methyl-4H- pyrimido[1,2-b]pyridazin-4-one | 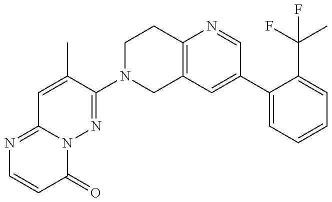 [SMILES: Cc1cc2nccc(=O)n2nc1N1CCc2ncc(-c3ccccc3C(C)(F)F)cc2C1] | 434 |
| 1-434 | 7-(3-(2-(difluoromethyl)phenyl)- 7,8-dihydro-1,6-naphthyridin- 6(5H)-yl)-8-methyl-4H- pyrimido[1,2-b]pyridazin-4-one | 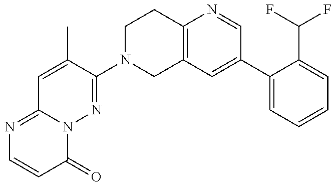 [SMILES: Cc1cc2nccc(=O)n2nc1N1CCc2ncc(-c3ccccc3C(F)F)cc2C1] | 420 |
| 1-435 | 8-methyl-7-(3-(3-methyl-1H-indol- 4-yl)-7,8-dihydro-1,6-naphthyridin- 6(5H)-yl)-4H-pyrimido[1,2- b]pyridazin-4-one | 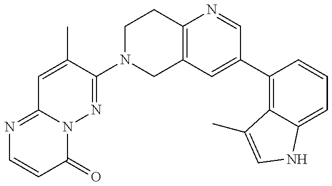 [SMILES: Cc1cc2nccc(=O)n2nc1N1CCc2ncc(-c3cccc4[nH]cc(C)c34)cc2C1] | 423 |
| 1-436 | 7-(3-(2,3-dihydro-1H-inden-4-yl)- 7,8-dihydro-1,6-naphthyridin- 6(5H)-yl)-8-methyl-4H- pyrimido[1,2-b]pyridazin-4-one | 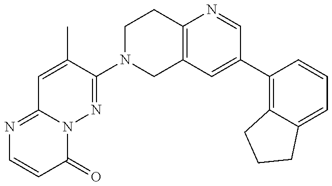 [SMILES: Cc1cc2nccc(=O)n2nc1N1CCc2ncc(-c3cccc4c3CCC4)cc2C1] | 410 |
| 1-437 | 2-methyl-3-(6-(8-methyl-4-oxo-4H- pyrimido[1,2-b]pyridazin-7-yl)- 5,6,7,8-tetrahydro-1,6-naphthyridin- 3-yl)benzonitrile | 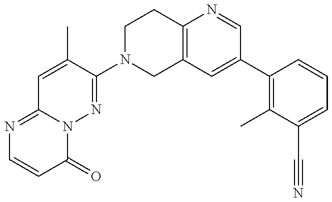 [SMILES: Cc1cc2nccc(=O)n2nc1N1CCc2ncc(-c3cccc(C#N)c3C)cc2C1] | 409 |
| 1-438 | 1-(2-(6-(8-methyl-4-oxo-4H- pyrimido[1,2-b]pyridazin-7-yl)- 5,6,7,8-tetrahydro-1,6-naphthyridin- 3-yl)phenyl)cyclopropane-1- carbonitrile | 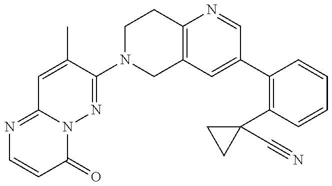 [SMILES: Cc1cc2nccc(=O)n2nc1N1CCc2ncc(-c3ccccc3C3(C#N)CC3)cc2C1] | 435 |
| 1-439 | 7-(3-(2-isopropylphenyl)-7,8- dihydro-1,6-naphthyridin- 6(5H)-yl)-8-methyl-4H-pyrimido [1,2-b]pyridazin-4-one | 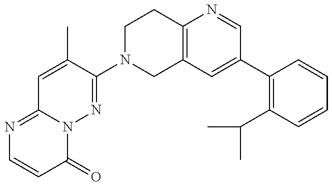 [SMILES: Cc1cc2nccc(=O)n2nc1N1CCc2ncc(-c3ccccc3C(C)C)cc2C1] | 412 |
| 1-440 | 7-(3-(2-cyclopropylphenyl)-7,8- dihydro-1,6-naphthyridin- 6(5H)-yl)-8-methyl-4H-pyrimido [1,2-b]pyridazin-4-one | 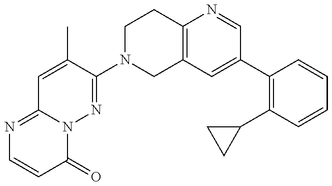 [SMILES: Cc1cc2nccc(=O)n2nc1N1CCc2ncc(-c3ccccc3C3CC3)cc2C1] | 410 |
| 1-441 | 7-(3-(3-chloro-2-methylphenyl)- 7,8-dihydro-1,6-naphthyridin- 6(5H)-yl)-8-methyl-4H- pyrimido[1,2-b]pyridazin-4-one | 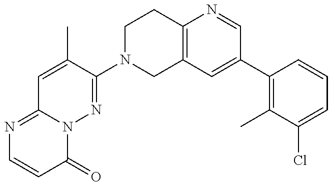 [SMILES: Cc1cc2nccc(=O)n2nc1N1CCc2ncc(-c3cccc(Cl)c3C)cc2C1] | 418 |
| 1-442 | 7-(3-(2,3-dimethylphenyl)-7,8- dihydro-1,6-naphthyridin- 6(5H)-yl)-8-methyl-4H-pyrimido [1,2-b]pyridazin-4-one | 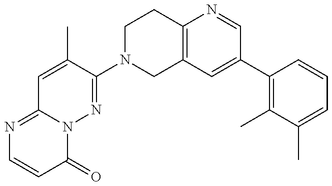 [SMILES: Cc1cc2nccc(=O)n2nc1N1CCc2ncc(-c3cccc(C)c3C)cc2C1] | 398 |
| 1-443 | 7-(3-(4-fluoro-2-methylphenyl)-7,8- dihydro-1,6-naphthyridin- 6(5H)-yl)-8-methyl-4H-pyrimido [1,2-b]pyridazin-4-one | 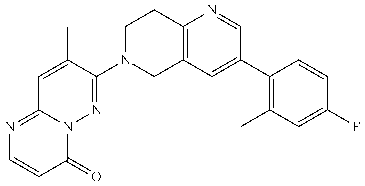 [SMILES: Cc1cc(F)ccc1-c1cnc2c(c1)CN(c1nn3c(=O)ccnc3cc1C)CC2] | 402 |
| 1-444 | 7-(3-(3,5-difluoro-4-methoxy-2- methylphenyl)-7,8-dihydro- 1,6-naphthyridin- 6(5H)-yl)-8-methyl-4H-pyrimido [1,2-b]pyridazin-4-one | 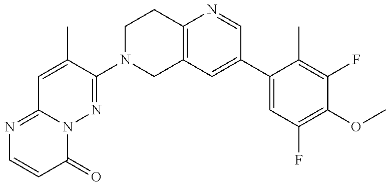 [SMILES: COc1c(F)cc(-c2cnc3c(c2)CN(c2nn4c(=O)ccnc4cc2C)CC3)c(C)c1F] | 450 |
| 1-445 | 7-(3-(4,5-difluoro-2-methylphenyl)- 7,8-dihydro-1,6-naphthyridin- 6(5H)-yl)-8-methyl-4H- pyrimido[1,2-b]pyridazin-4-one | 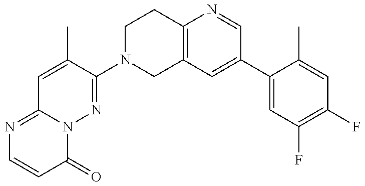 [SMILES: Cc1cc(F)c(F)cc1-c1cnc2c(c1)CN(c1nn3c(=O)ccnc3cc1C)CC2] | 420.2 |
| 1-446 | 7-(3-(4,5-difluoro-2-methylphenyl)- 7,8-dihydro-1,6-naphthyridin- 6(5H)-yl)-8,9-dimethyl-4H- pyrimido[1,2-b]pyridazin-4-one | 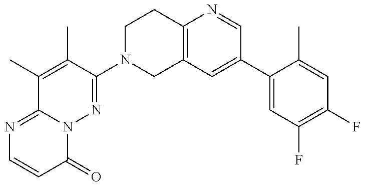 [SMILES: Cc1cc(F)c(F)cc1-c1cnc2c(c1)CN(c1nn3c(=O)ccnc3c(C)c1C)CC2] | 434.3 |
| 1-447 | 7-(3-(3,5-difluoro-2-methylphenyl)- 7,8-dihydro-1,6-naphthyridin- 6(5H)-yl)-8-methyl-4H-pyrimido [1,2-b]pyridazin-4-one | 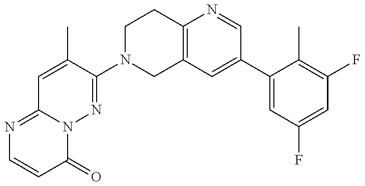 [SMILES: Cc1cc2nccc(=O)n2nc1N1CCc2ncc(-c3cc(F)cc(F)c3C)cc2C1] | 420.2 |
| 1-448 | 8-methyl-7-(2-methyl-7,8-dihydro- 1,6-naphthyridin- 6(5H)-yl)-4H-pyrimido[1,2-b] pyridazin-4-one | 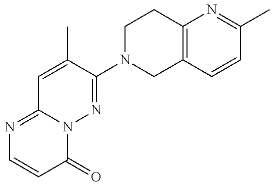 [SMILES: Cc1ccc2c(n1)CCN(c1nn3c(=O)ccnc3cc1C)C2] | 308 |
| 1-449 | 8-methyl-7-(3-(1-methyl-1H- pyrazol-5-yl)-7,8-dihydro- 1,6-naphthyridin- 6(5H)-yl-5,5,7,7-d₄)-4H- pyrimido[1,2-b]pyridazin-4-one | 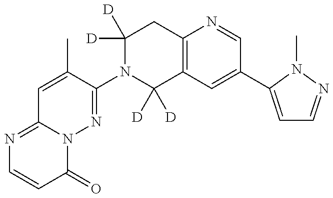 [SMILES: [2H]C1([2H])Cc2ncc(-c3ccnn3C)cc2C([2H])([2H])N1c1nn2c(=O)ccnc2cc1C] | 378 |
| 1-450 | 7-(3-(3,5-dimethylisoxazol-4-yl)- 7,8-dihydro-1,6-naphthyridin- 6(5H)-yl-5,5,7,7-d₄)-8-methyl-4H- pyrimido[1,2-b]pyridazin-4-one | 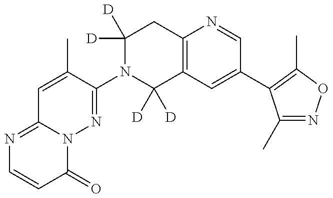 [SMILES: [2H]C1([2H])Cc2ncc(-c3c(C)noc3C)cc2C([2H])([2H])N1c1nn2c(=O)ccnc2cc1C] | 393 |

^{a}First eluting enantiomer by chiral SFC separation performed on a PIC Solution SFC-PICLab PREP 100. Column: Chiral Technologies CHIRALPAK IA,
20 × 250 mm, 5 μm.
^{b}Second eluting enantiomer by chiral SFC separation performed on a PIC Solution SFC-PICLab PREP 100. Column: Chiral Technologies CHIRALPAK
IA, 20 × 250 mm, 5 μm.

c. Exemplified Compounds of the Invention

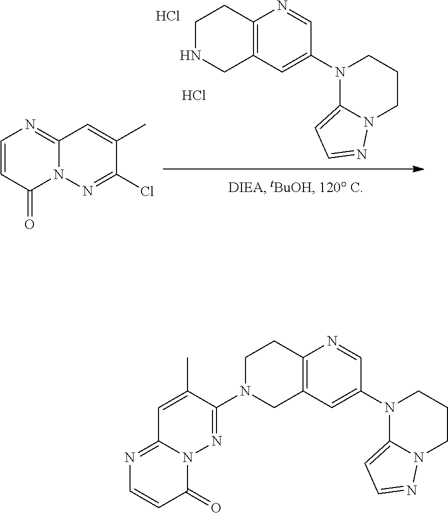 [SMILES: Cc1cc2nccc(=O)n2nc1Cl ; Cl ; Cl ; c1cc2n(n1)CCCN2c1cnc2c(c1)CNCC2 ; Cc1cc2nccc(=O)n2nc1N1CCc2ncc(N3CCCn4nccc43)cc2C1]

7-(3-(6,7-Dihydropyrazolo[1,5-a]pyrimidin-4(5H)-yl)-7,8-dihydro-1,6-naphthyridin-6(5H)-yl)-8-methyl-4H-pyrimido[1,2-b]pyridazin-4-one (Compound 2-1). A sealed tube containing a solution of 7-chloro-8-methyl-4H-pyrimido[1,2-b]pyridazin-4-one (15 mg), 3-(6,7-dihydropyrazolo[1,5-a]pyrimidin-4(5H)-yl)-5,6,7,8-tetrahydro-1,6-naphthyridine dihydrochloride (25 mg) and DIEA (67 L) in tert-butanol (1 mL) was heated to 120° C. for 18 h. The reaction mixture was cooled to ambient temperature, diluted with 1 mL of DMSO, and purified via RP-HPLC (5-45% MeCN/0.05% aqueous NH₄OH) to afford the title compound that contained impurities. The material was repurified using RP-HPLC (0-30% MeCN/0.1% aqueous TFA). The fractions containing the title compound were basified with saturated aqueous NaHCO₃ and extracted with chloroform/IPA (4:1) (3×). The combined organics were passed through a phase separator and concentrated to give 9 mg of the title compound. ES-MS [M+1]⁺: 415.2. ¹H NMR (400 MHz, CDCl₃) δ 8.50 (d, J=2.6 Hz, 1H), 8.34 (s, 1H), 7.59 (q, J=1.2 Hz, 1H), 7.46 (d, J=2.6 Hz, 1H), 7.33 (d, J=2.1 Hz, 1H), 5.65 (d, J=2.1 Hz, 1H), 4.70 (s, 2H), 4.24 (t, J=6.2 Hz, 2H), 3.88-3.69 (m, 2H), 3.65 (t, J=5.9 Hz, 2H), 3.23 (t, J=5.9 Hz, 2H), 2.54 (d, J=1.2 Hz, 3H), 2.35 (p, J=6.1 Hz, 2H); ES-MS [M+1]⁺: 449.2.

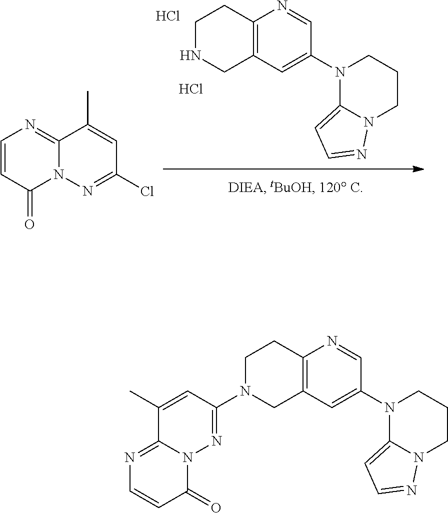 [SMILES: Cc1cc(Cl)nn2c(=O)ccnc12 ; Cl ; Cl ; c1cc2n(n1)CCCN2c1cnc2c(c1)CNCC2 ; Cc1cc(N2CCc3ncc(N4CCCn5nccc54)cc3C2)nn2c(=O)ccnc12]

7-(3-(6,7-Dihydropyrazolo[1,5-a]pyrimidin-4(5H)-yl)-7,8-dihydro-1,6-naphthyridin-6(5H)-yl)-9-methyl-4H-pyrimido[1,2-b]pyridazin-4-one (Compound 2-3). Prepared in a similar manner as Compound 2-1 to afford 11.1 mg of the title compound. ¹H NMR (400 MHz, CDCl₃) δ 8.49 (d, J=2.6 Hz, 1H), 8.13 (d, J=6.2 Hz, 1H), 7.49 (d, J=2.7 Hz, 1H), 7.33 (d, J=2.0 Hz, 1H), 7.29 (d, J=1.3 Hz, 1H), 6.60 (d, J=6.2 Hz, 1H), 5.65 (d, J=2.1 Hz, 1H), 4.92 (s, 2H), 4.24 (t, J=6.2 Hz, 2H), 3.97 (t, J=5.9 Hz, 2H), 3.74-3.67 (m, 2H), 3.17 (t, J=5.9 Hz, 2H), 2.59 (d, J=1.2 Hz, 3H), 2.34 (p, J=6.0 Hz, 2H); ES-MS [M+1]⁺: 415.2.

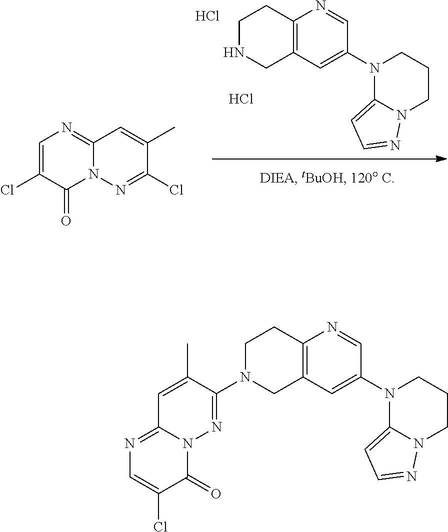 [SMILES: Cc1cc2ncc(Cl)c(=O)n2nc1Cl ; Cl ; Cl ; c1cc2n(n1)CCCN2c1cnc2c(c1)CNCC2 ; Cc1cc2ncc(Cl)c(=O)n2nc1N1CCc2ncc(N3CCCn4nccc43)cc2C1]

3-Chloro-7-(3-(6,7-dihydropyrazolo[1,5-a]pyrimidin-4(5H)-yl)-7,8-dihydro-1,6-naphthyridin-6(5H)-yl)-8-methyl-4H-pyrimido[1,2-b]pyridazin-4-one (Compound 2-4). Prepared in a similar manner as Compound 2-1 to afford 6.7 mg of the title compound (23% yield). ES-MS [M+1]⁺: 449.2. ¹H NMR (400 MHz, CDCl₃) δ 8.50 (d, J=2.6 Hz, 1H), 8.34 (s, 1H), 7.59 (q, J=1.2 Hz, 1H), 7.46 (d, J=2.6 Hz, 1H), 7.33 (d, J=2.1 Hz, 1H), 5.65 (d, J=2.1 Hz, 1H), 4.70 (s, 2H), 4.24 (t, J=6.2 Hz, 2H), 3.88-3.69 (m, 2H), 3.65 (t, J=5.9 Hz, 2H), 3.23 (t, J=5.9 Hz, 2H), 2.54 (d, J=1.2 Hz, 3H), 2.35 (p, J=6.1 Hz, 2H); ES-MS [M+1]⁺: 449.2.

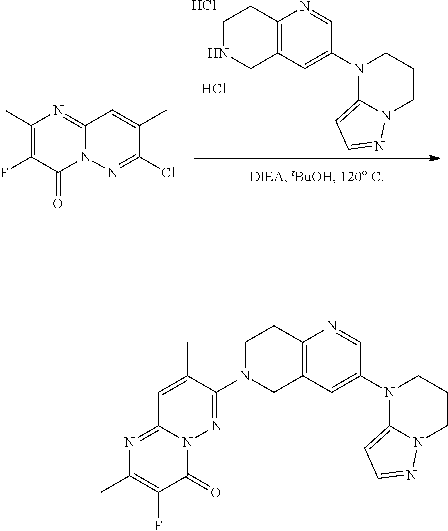 [SMILES: Cl ; Cc1cc2nc(C)c(F)c(=O)n2nc1Cl ; Cl ; c1cc2n(n1)CCCN2c1cnc2c(c1)CNCC2 ; Cc1cc2nc(C)c(F)c(=O)n2nc1N1CCc2ncc(N3CCCn4nccc43)cc2C1]

7-(3-(6,7-Dihydropyrazolo[1,5-a]pyrimidin-4(5H)-yl)-7,8-dihydro-1,6-naphthyridin-6(5H)-yl)-3-fluoro-2,8-dimethyl-4H-pyrimido[1,2-b]pyridazin-4-one (Compound 2-9). Prepared in a similar manner as Compound 2-1 to afford 14.5 mg of the title compound. ¹H NMR (400 MHz, CDCl₃) δ 8.50 (d, J=2.6 Hz, 1H), 7.51 (q, J=1.4 Hz, 1H), 7.46 (d, J=2.6 Hz, 1H), 7.33 (d, J=2.1 Hz, 1H), 5.65 (d, J=2.1 Hz, 1H), 4.70 (s, 2H), 4.24 (t, J=6.2 Hz, 2H), 3.75-3.67 (m, 2H), 3.62 (t, J=5.9 Hz, 2H), 3.22 (t, J=5.9 Hz, 2H), 2.51 (s, 3H), 2.48 (d, J=3.5 Hz, 3H), 2.35 (p, J=6.1 Hz, 2H); ES-MS [M+1]⁺: 447.2.

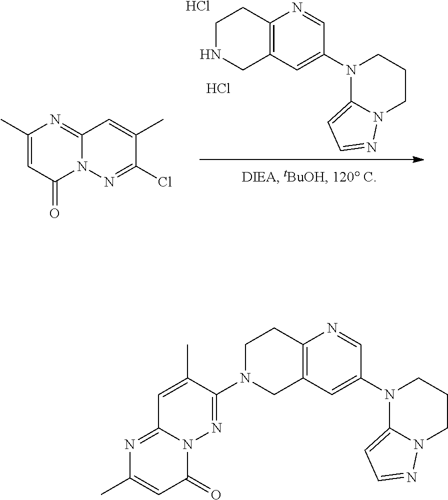 [SMILES: Cl ; Cc1cc(=O)n2nc(Cl)c(C)cc2n1 ; Cl ; c1cc2n(n1)CCCN2c1cnc2c(c1)CNCC2 ; Cc1cc(=O)n2nc(N3CCc4ncc(N5CCCn6nccc65)cc4C3)c(C)cc2n1]

7-(3-(6,7-Dihydropyrazolo[1,5-a]pyrimidin-4(5H)-yl)-7,8-dihydro-1,6-naphthyridin-6(5H)-yl)-2,8-dimethyl-4H-pyrimido[1,2-b]pyridazin-4-one (Compound 2-10). Prepared in a similar manner as Compound 2-1 to afford 14.9 mg of the title compound. ¹H NMR (400 MHz, CDCl₃) δ 8.49 (d, J=2.6 Hz, 1H), 7.52 (d, J=1.3 Hz, 1H), 7.45 (d, J=2.6 Hz, 1H), 7.32 (d, J=2.0 Hz, 1H), 6.45 (d, J=0.8 Hz, 1H), 5.64 (d, J=2.0 Hz, 1H), 4.69 (s, 2H), 4.24 (t, J=6.2 Hz, 2H), 3.74-3.67 (m, 2H), 3.60 (t, J=5.9 Hz, 2H), 3.22 (t, J=5.9 Hz, 2H), 2.51 (d, J=1.2 Hz, 3H), 2.42 (s, 3H), 2.34 (p, J=6.0 Hz, 2H); ES-MS [M+1]⁺: 429.2.

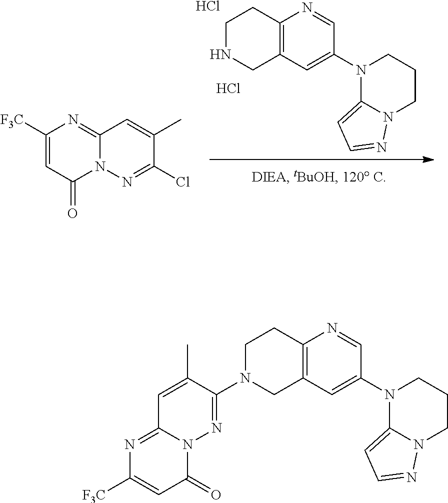 [SMILES: Cl ; Cc1cc2nc(C(F)(F)F)cc(=O)n2nc1Cl ; Cl ; c1cc2n(n1)CCCN2c1cnc2c(c1)CNCC2 ; Cc1cc2nc(C(F)(F)F)cc(=O)n2nc1N1CCc2ncc(N3CCCn4nccc43)cc2C1]

7-(3-(6,7-Dihydropyrazolo[1,5-a]pyrimidin-4(5H)-yl)-7,8-dihydro-1,6-naphthyridin-6(5H)-yl)-8-methyl-2-(trifluoromethyl)-4H-pyrimido[1,2-b]pyridazin-4-one (Compound 2-11). Prepared in a similar manner as Compound 2-1 to afford 15.3 mg of the title compound. ES-MS [M+1]⁺: 483.2. ¹H NMR (400 MHz, CDCl₃) δ 8.50 (d, J=2.6 Hz, 1H), 7.69 (q, J=1.1 Hz, 1H), 7.46 (d, J=2.6 Hz, 1H), 7.33 (d, J=2.0 Hz, 1H), 6.92 (s, 1H), 5.65 (d, J=2.1 Hz, 1H), 4.71 (s, 2H), 4.24 (t, J=6.2 Hz, 2H), 3.75-3.69 (m, 2H), 3.67 (t, J=5.9 Hz, 2H), 3.24 (t, J=5.9 Hz, 2H), 2.57 (d, J=1.2 Hz, 3H), 2.35 (p, J=6.0 Hz, 2H); ES-MS [M+1]⁺: 483.2.

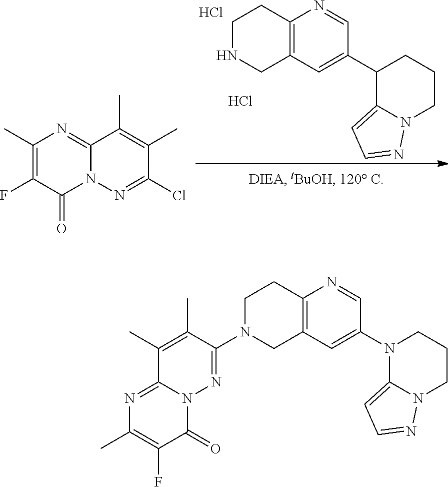 [SMILES: Cc1nc2c(C)c(C)c(Cl)nn2c(=O)c1F ; Cl ; c1cc2n(n1)CCCC2c1cnc2c(c1)CNCC2 ; Cl ; Cc1nc2c(C)c(C)c(N3CCc4ncc(N5CCCn6nccc65)cc4C3)nn2c(=O)c1F]

7-(3-(6,7-Dihydropyrazolo[1,5-a]pyrimidin-4(5H)-yl)-7,8-dihydro-1,6-naphthyridin-6(5H)-yl)-3-fluoro-2,8,9-trimethyl-4H-pyrimido[1,2-b]pyridazin-4-one (Compound 2-12). Prepared in a similar manner as Compound 2-1 to afford 8.7 mg of the title compound. ¹H NMR (400 MHz, MeOD) δ 8.41 (d, J=2.6 Hz, 1H), 7.69 (d, J=2.6 Hz, 1H), 7.28 (d, J=2.1 Hz, 1H), 5.68 (d, J=2.2 Hz, 1H), 4.65 (s, 2H), 4.18 (t, J=6.2 Hz, 2H), 3.80-3.73 (m, 2H), 3.64 (t, J=5.9 Hz, 2H), 3.19 (t, J=5.9 Hz, 2H), 2.59 (d, J=0.6 Hz, 3H), 2.51 (d, J=3.5 Hz, 3H), 2.48 (s, 3H), 2.34 (p, J=6.0 Hz, 2H); ES-MS [M+1]⁺: 461.2.

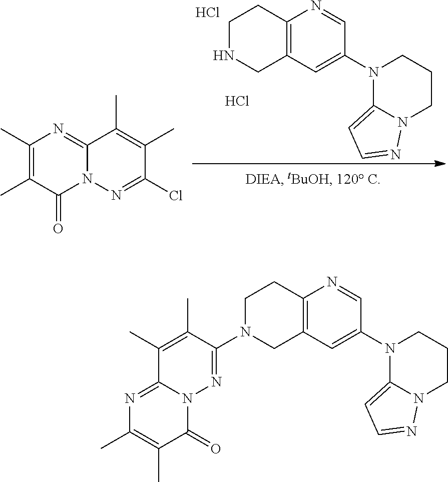 [SMILES: Cc1nc2c(C)c(C)c(Cl)nn2c(=O)c1C ; Cl ; c1cc2n(n1)CCCN2c1cnc2c(c1)CNCC2 ; Cl ; Cc1nc2c(C)c(C)c(N3CCc4ncc(N5CCCn6nccc65)cc4C3)nn2c(=O)c1C]

7-(3-(6,7-Dihydropyrazolo[1,5-a]pyrimidin-4(5H)-yl)-7,8-dihydro-1,6-naphthyridin-6(5H)-yl)-2,3,8-trimethyl-4H-pyrimido[1,2-b]pyridazin-4-one (Compound 2-13). Prepared in a similar manner as Compound 2-1 to afford 7.9 mg of the title compound. ¹H NMR (400 MHz, CDCl₃) δ 8.49 (d, J=2.6 Hz, 1H), 7.47 (q, J=1.1 Hz, 1H), 7.44 (d, J=2.6 Hz, 1H), 7.32 (d, J=2.0 Hz, 1H), 5.64 (d, J=2.0 Hz, 1H), 4.68 (s, 2H), 4.24 (t, J=6.2 Hz, 2H), 3.74-3.67 (m, 3H), 3.59 (t, J=5.9 Hz, 2H), 3.21 (t, J=5.9 Hz, 2H), 2.49 (d, J=1.2 Hz, 3H), 2.45 (s, 3H), 2.35 (p, J=6.0 Hz, 2H), 2.25 (s, 3H); ES-MS [M+1]⁺: 443.2.

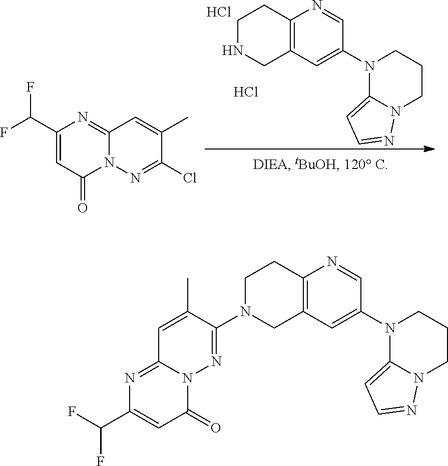 [SMILES: Cc1cc2nc(C(F)F)cc(=O)n2nc1Cl ; Cl ; c1cc2n(n1)CCCN2c1cnc2c(c1)CNCC2 ; Cl ; Cc1cc2nc(C(F)F)cc(=O)n2nc1N1CCc2ncc(N3CCCn4nccc43)cc2C1]

2-(Difluoromethyl)-7-(3-(6,7-dihydropyrazolo[1,5-a]pyrimidin-4(5H)-yl)-7,8-dihydro-1,6-naphthyridin-6(5H)-yl)-8-methyl-4H-pyrimido[1,2-b]pyridazin-4-one (Compound 2-14). Prepared in a similar manner as Compound 2-1 to afford 10.8 mg of the title compound. ¹H NMR (400 MHz, CDCl₃) δ 8.49 (d, J=2.6 Hz, 1H), 7.47 (q, J=1.1 Hz, 1H), 7.44 (d, J=2.6 Hz, 1H), 7.32 (d, J=2.0 Hz, 1H), 5.64 (d, J=2.0 Hz, 1H), 4.68 (s, 2H), 4.24 (t, J=6.2 Hz, 2H), 3.74-3.67 (m, 3H), 3.59 (t, J=5.9 Hz, 2H), 3.21 (t, J=5.9 Hz, 2H), 2.49 (d, J=1.2 Hz, 3H), 2.45 (s, 3H), 2.35 (p, J=6.0 Hz, 2H), 2.25 (s, 3H); ES-MS [M+1]⁺: 465.2.

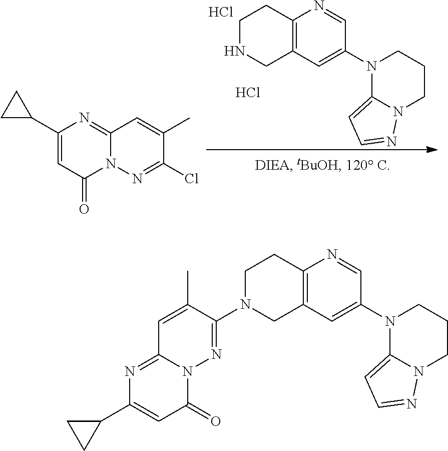 [SMILES: Cc1cc2nc(C3CC3)cc(=O)n2nc1Cl ; Cl ; c1cc2n(n1)CCCN2c1cnc2c(c1)CNCC2 ; Cl ; Cc1cc2nc(C3CC3)cc(=O)n2nc1N1CCc2ncc(N3CCCn4nccc43)cc2C1]

2-Cyclopropyl-7-(3-(6,7-dihydropyrazolo[1,5-a]pyrimidin-4(5H)-yl)-7,8-dihydro-1,6-naphthyridin-6(5H)-yl)-8-methyl-4H-pyrimido[1,2-b]pyridazin-4-one (Compound 2-15). Prepared in a similar manner as Compound 2-1 to afford 6.0 mg of the title compound. ¹H NMR (400 MHz, CDCl₃) δ 8.49 (d, J=2.6 Hz, 1H), 7.46 (q, J=1.1 Hz, 1H), 7.45 (d, J=2.6 Hz, 1H), 7.32 (d, J=2.0 Hz, 1H), 6.45 (s, 1H), 5.64 (d, J=2.0 Hz, 1H), 4.67 (s, 2H), 4.24 (t, J=6.2 Hz, 2H), 3.74-3.66 (m, 2H), 3.58 (t, J=5.9 Hz, 2H), 3.21 (t, J=5.8 Hz, 2H), 2.49 (d, J=1.2 Hz, 3H), 2.34 (p, J=6.1 Hz, 2H), 2.05-1.81 (m, 1H), 1.15-1.07 (m, 2H), 1.06-0.95 (m, 2H); ES-MS [M+1]⁺: 455.2.

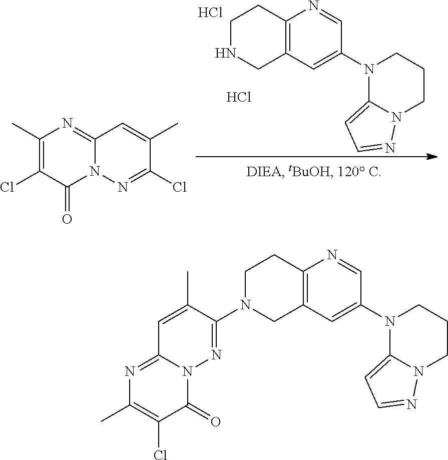 [SMILES: Cc1cc2nc(C)c(Cl)c(=O)n2nc1Cl ; Cl ; c1cc2n(n1)CCCN2c1cnc2c(c1)CNCC2 ; Cl ; Cc1cc2nc(C)c(Cl)c(=O)n2nc1N1CCc2ncc(N3CCCn4nccc43)cc2C1]

3-Chloro-7-(3-(6,7-dihydropyrazolo[1,5-a]pyrimidin-4(5H)-yl)-7,8-dihydro-1,6-naphthyridin-6(5H)-yl)-2,8-dimethyl-4H-pyrimido[1,2-b]pyridazin-4-one (Compound 2-16). Prepared in a similar manner as Compound 2-1 to afford 13.4 mg of the title compound. ¹H NMR (400 MHz, CDCl₃) δ 8.50 (d, J=2.6 Hz, 1H), 7.53 (q, J=1.1 Hz, 1H), 7.46 (d, J=2.6 Hz, 1H), 7.32 (d, J=2.0 Hz, 1H), 5.65 (d, J=2.1 Hz, 1H), 4.70 (s, 2H), 4.24 (t, J=6.2 Hz, 2H), 3.75-3.67 (m, 2H), 3.63 (t, J=5.9 Hz, 2H), 3.22 (t, J=5.9 Hz, 2H), 2.58 (s, 3H), 2.52 (d, J=1.2 Hz, 3H), 2.35 (p, J=6.1 Hz, 2H); ES-MS [M+1]⁺: 463.2.

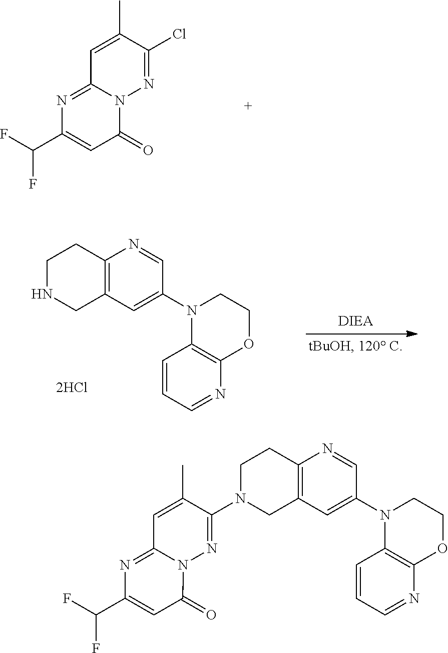 [SMILES: Cc1cc2nc(C(F)F)cc(=O)n2nc1Cl ; c1cnc2c(c1)N(c1cnc3c(c1)CNCC3)CCO2 ; Cc1cc2nc(C(F)F)cc(=O)n2nc1N1CCc2ncc(N3CCOc4ncccc43)cc2C1]

2-(Difluoromethyl)-7-(3-(2,3-dihydro-1H-pyrido[2,3-b][1,4]oxazin-1-yl)-7,8-dihydro-1,6-naphthyridin-6(5H)-yl)-8-methyl-4H-pyrimido[1,2-b]pyridazin-4-one (Compound 2-29) To a solution of 7-chloro-2-(difluoromethyl)-8-methyl-pyrimido[1,2-b]pyridazin-4-one (10 mg, 0.04 mmol) in tert-butanol (1.0 mL) was added DIEA (0.035 mL, 0.20 mmol) and 1-(5,6,7,8-tetrahy dro-1,6-naphthyridin-3-yl)-2,3-dihydropyrido[2,3-b][1,4]oxazine dihydrochloride (21 mg, 0.06 mmol). The mixture was heated to 120° C. for 18 h. Solvents were removed, the crude residue was dissolved in DMSO (2 mL) and purified by reverse-phase chromatography (MeCN/0.1% aqueous TFA) to afford the title compound (100 mg). ¹H NMR (400 MHz, CDCl₃) δ 8.43 (d, J=2.6 Hz, 1H), 7.77 (dd, J=4.8, 1.6 Hz, 1H), 7.64 (d, J=1.3 Hz, 1H), 7.44 (d, J=2.6 Hz, 1H), 7.16 (dd, J=7.9, 1.6 Hz, 1H), 6.84 (s, 1H), 6.78 (dd, J=7.9, 4.8 Hz, 1H), 6.46 (s, 1H), 4.71 (s, 2H), 4.55-4.48 (m, 2H), 3.77-3.71 (m, 2H), 3.67 (t, J=5.9 Hz, 2H), 3.25 (t, J=5.9 Hz, 2H), 2.56 (d, J=1.2 Hz, 3H); ES-MS [M+H]⁺=478.2.

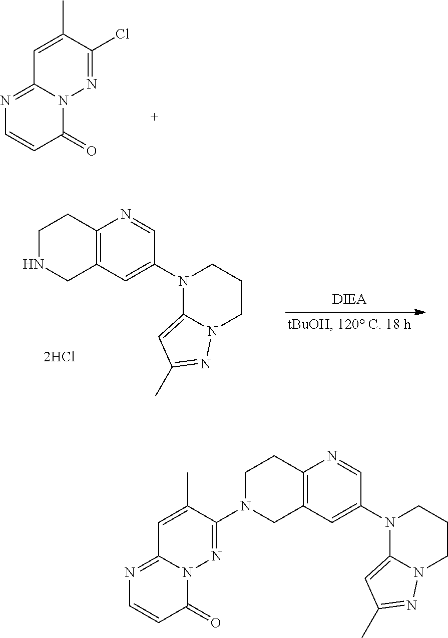 [SMILES: Cc1cc2nccc(=O)n2nc1Cl ; Cc1cc2n(n1)CCCN2c1cnc2c(c1)CNCC2 ; Cc1cc2n(n1)CCCN2c1cnc2c(c1)CN(c1nn3c(=O)ccnc3cc1C)CC2]

8-Methyl-7-(3-(2-methyl-6,7-dihydropyrazolo[1,5-a]pyrimidin-4(5H)-yl)-7,8-dihydro-1,6-naphthyridin-6(5H)-yl)-4H-pyrimido[1,2-b]pyridazin-4-one (Compound 2-5) Prepared in a similar manner as compound 2-29. ¹H NMR (400 MHz, DMSO) δ 8.40 (d, J=2.6 Hz, 1H), 8.13 (d, J=6.3 Hz, 1H), 7.79 (d, J=1.4 Hz, 1H), 7.60 (d, J=2.7 Hz, 1H), 6.43 (d, J=6.3 Hz, 1H), 5.49 (s, 1H), 4.57 (s, 2H), 4.00 (t, J=6.2 Hz, 2H), 3.65 (q, J=6.1 Hz, 4H), 3.10 (t, J=5.9 Hz, 2H), 2.51 (s, 3H), 2.22-2.14 (m, 2H), 2.02 (s, 3H); ES-MS [M+H]⁺=429.4.

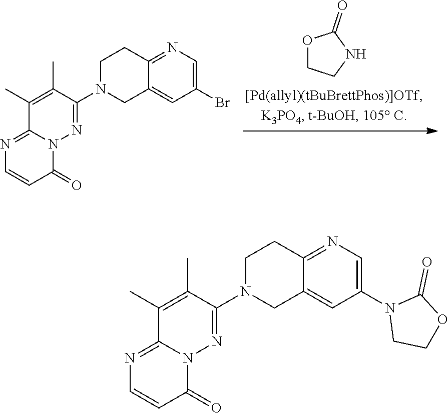 [SMILES: Cc1c(N2CCc3ncc(Br)cc3C2)nn2c(=O)ccnc2c1C ; O=C1NCCO1 ; Cc1c(N2CCc3ncc(N4CCOC4=O)cc3C2)nn2c(=O)ccnc2c1C]

3-(6-(8,9-Dimethyl-4-oxo-4H-pyrimido[1,2-b]pyridazin-7-yl)-5,6,7,8-tetrahydro-1,6-naphthyridin-3-yl)oxazolidin-2-one (Compound 2-144). 7-(3-Bromo-7,8-dihydro-1,6-naphthyridin-6(5H)-yl)-8,9-dimethyl-4H-pyrimido[1,2-b]pyridazin-4-one (20 mg, 0.052 mmol), oxazolidin-2-one (14 mg, 0.16 mmol), potassium phosphate tribasic (19 mg, 0.088 mmol) and [Pd(allyl)(tBuBrettPhos)]OTf (4.0 mg, 0.005 mmol) were combined in a vial, which was sealed and placed under an inert atmosphere. t-BuOH (0.75 mL) was then added via syringe, and the resulting reaction mixture was stirred at 105° C. for 3 h, after which time the reaction mixture was cooled to ambient temperature, and the solvents were concentrated. Solids were removed by syringe filtration, and crude residue was purified by RP-HPLC (2-32% MeCN/0.1% aqueous TFA). The fractions containing product were basified with sat. NaHCO₃ solution, and extracted with DCM. The combined organic extracts were filtered through a phase separator, and concentrated to afford 7.9 mg of the title compound. ¹H NMR (400 MHz, CDCl₃) δ 8.63 (d, J=2.6 Hz, 1H), 8.17 (d, J=6.3 Hz, 1H), 7.87 (d, J=2.6 Hz, 1H), 6.57 (d, J=6.3 Hz, 1H), 4.65 (s, 2H), 4.57-4.53 (m, 2H), 4.11-4.07 (m, 2H), 3.56 (t, J=5.9 Hz, 2H), 3.23 (t, J=5.9 Hz, 2H), 2.56 (d, J=0.9 Hz, 3H), 2.44 (d, J=1.0 Hz, 3H); ES-MS [M+1]⁺: 393.1.

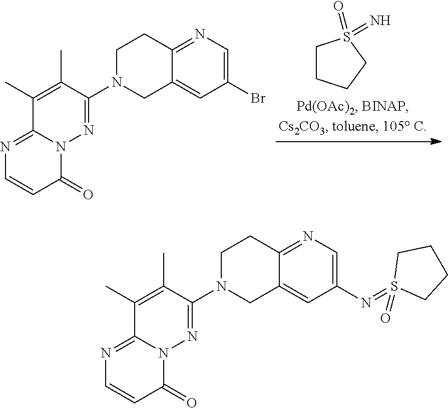 [SMILES: Cc1c(N2CCc3ncc(Br)cc3C2)nn2c(=O)ccnc2c1C ; N=S1(=O)CCCC1 ; Cc1c(N2CCc3ncc(N=S4(=O)CCCC4)cc3C2)nn2c(=O)ccnc2c1C]

8,9-Dimethyl-7-(3-((1-oxidotetrahydro-1λ⁶-thiophen-1-ylidene)amino)-7,8-dihydro-1,6-naphthyridin-6(5H)-yl)-4H-pyrimido[1,2-b]pyridazin-4-one (Compound 2-147). 7-(3-Bromo-7,8-dihydro-1,6-naphthyridin-6(5H)-yl)-8,9-dimethyl-4H-pyrimido[1,2-b]pyridazin-4-one (20 mg, 0.052 mmol), 1-iminotetrahydro-1H-1)⁶-thiophene 1-oxide (19 mg, 0.16 mmol), cesium carbonate (24 mg, 0.072 mmol) rac-BINAP (2.8 mg, 0.008 mmol) and palladium(II) acetate (1.1 mg, 0.005 mmol) were combined in a vial, which was sealed and placed under an inert atmosphere. Toluene (1 mL) was then added via syringe, and the resulting reaction mixture was stirred at 105° C. for 3 h, after which time the reaction mixture was cooled to ambient temperature, and the solvents were concentrated. Solids were removed by syringe filtration, and crude residue was purified by RP-HPLC (2-32% MeCN/0.1% aqueous TFA). The fractions containing product were basified with sat. NaHCO₃ solution and extracted with DCM. The combined organic extracts were filtered through a phase separator, and concentrated to afford 5.7 mg of the title compound. ¹H NMR (400 MHz, CDCl₃) δ 8.22 (d, J=2.5 Hz, 1H), 8.17 (d, J=6.3 Hz, 1H), 7.26 (d, J=2.6 Hz, 1H), 6.57 (d, J=6.3 Hz, 1H), 4.59 (s, 2H), 3.52-3.48 (m, 2H), 3.43-3.36 (m, 2H), 3.24-3.16 (m, 4H), 2.56 (d, J=1.0 Hz, 3H), 2.43 (d, J=1.0 Hz, 3H), 2.39-2.23 (m, 4H); ES-MS [M+1]⁺: 425.2.

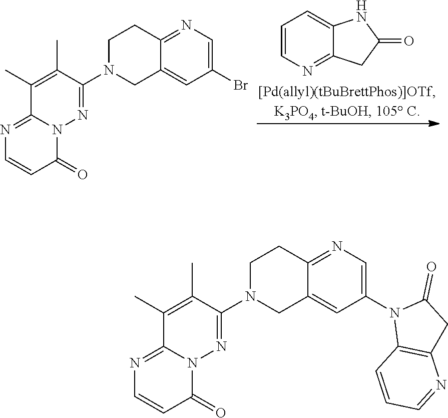 [SMILES: Cc1c(N2CCc3ncc(Br)cc3C2)nn2c(=O)ccnc2c1C ; O=C1Cc2ncccc2N1 ; Cc1c(N2CCc3ncc(N4C(=O)Cc5ncccc54)cc3C2)nn2c(=O)ccnc2c1C]

8,9-Dimethyl-7-(3-(2-oxo-2,3-dihydro-1H-pyrrolo[3,2-b]pyridin-1-yl)-7,8-dihydro-1,6-naphthyridin-6(5H)-yl)-4H-pyrimido[1,2-b]pyridazin-4-one (Compound 2-149). 7-(3-Bromo-7,8-dihydro-1,6-naphthyridin-6(5H)-yl)-8,9-dimethyl-4H-pyrimido[1,2-b]pyridazin-4-one (20 mg, 0.052 mmol), 1,3-dihydro-2H-pyrrolo[3,2-b]pyridin-2-one (21 mg, 0.16 mmol), potassium phosphate tribasic (19 mg, 0.088 mmol) and [Pd(allyl)(tBuBrettPhos)]Otf (4.0 mg, 0.005 mmol) were combined in a vial, which was sealed and placed under an inert atmosphere. t-BuOH (0.75 mL) was then added via syringe, and the resulting reaction mixture was stirred at 105° C. for 2 h, after which time the reaction mixture was cooled to ambient temperature, and the solvents were concentrated. Solids were removed by syringe filtration, and crude residue was purified by RP-HPLC (2-32% MeCN/0.1% aqueous TFA). The fractions containing product were basified with sat. NaHCO₃ solution and extracted with 3:1 chloroform/IPA solution (v v). Solvents were concentrated, and residue was re-purified by normal phase column chromatography on silica gel using 0-6% (1% NH₄OH in MeOH) in DCM to afford 6.8 mg of the title compound. ¹H NMR (400 MHz, MeOD) δ 8.85 (d, J=2.1 Hz, 1H), 8.29 (d, J=6.2 Hz, 1H), 7.99 (d, J=2.1 Hz, 1H), 7.48 (dd, J=6.6, 1.1 Hz, 1H), 7.16 (dd, J=7.3, 1.0 Hz, 1H), 6.70 (dd, J=7.3, 6.5 Hz, 1H), 6.59 (d, J=6.2 Hz, 1H), 4.69 (s, 2H), 3.67 (t, J=5.8 Hz, 2H), 3.34 (s, 2H), 3.22 (t, J=5.9 Hz, 2H), 2.60 (d, J=1.0 Hz, 3H), 2.53 (d, J=1.0 Hz, 3H); ES-MS [M+1]⁺: 440.2.

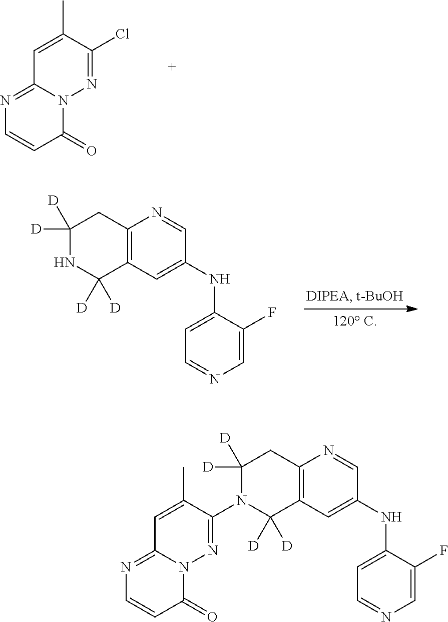 [SMILES: Cc1cc2nccc(=O)n2nc1Cl ; [2H]C1([2H])Cc2ncc(Nc3ccncc3F)cc2C([2H])([2H])N1 ; [2H]C1([2H])Cc2ncc(Nc3ccncc3F)cc2C([2H])([2H])N1c1nn2c(=O)ccnc2cc1C]

7-(3-((3-Fluoropyridin-4-yl)amino)-7,8-dihydro-1,6-naphthyridin-6(5H)-yl-5,5,7,7-d₄)-8-methyl-4H-pyrimido[1,2-b]pyridazin-4-one (Compound 2-164). In a vial were combined 7-chloro-8-methylpyrimido[1,2-b]pyridazin-4-one (15 mg, 0.08 mmol), N,N-diisopropylethylamine (67 uL, 0.38 mmol), and Intermediate A20 (19 mg, 0.08 mmol) in tert-butanol (0.8 mL). The reaction was heated to 120° C. After 18 h, the solvents were removed and the crude oil was purified by reverse-phase chromatography (5-45% MeCN/Water/0.05% NH₄OH) to afford the title compound. ¹H NMR (400 MHz, CDCl₃) δ 8.41 (d, J=2.4 Hz, 1H), 8.14 (s, 1H), 8.12 (s, 1H), 7.58 (d, J=1.1 Hz, 1H), 7.46 (d, J=2.5 Hz, 1H), 7.02-6.98 (m, 1H), 6.56 (d, J=6.4 Hz, 1H), 6.47 (d, J=2.1 Hz, 1H), 3.21 (s, 2H), 2.53 (d, J=0.9 Hz, 3H), 1.85 (s, 3H); ES-MS [M+1]⁺: 408.4.

The compounds shown in Table 2 may be prepared in an analogous manner with the appropriate starting materials.

TABLE 2

| Cpd. | | | ES-MS |
|---|---|---|---|
| No. | Name | Structure | [M + 1]⁺ |
| 2-1 | 7-(3-(6,7-dihydropyrazolo[1,5- a]pyrimidin-4(5H)-yl)-7,8-dihydro- 1,6-naphthyridin-6(5H)-yl)-8- methyl-4H-pyrimido[1,2- b]pyridazin-4-one | 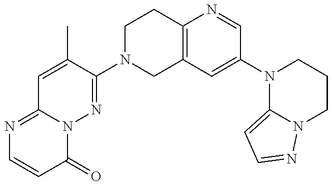 [SMILES: Cc1cc2nccc(=O)n2nc1N1CCc2ncc(N3CCCn4nccc43)cc2C1] | 415.2 |
| 2-2 | (R)-8-methyl-7-(3-(2- methylmorpholino)-7,8-dihydro- 1,6-naphthyridin-6(5H)-yl)-4H- pyrimido[1,2-b]pyridazin-4-one | 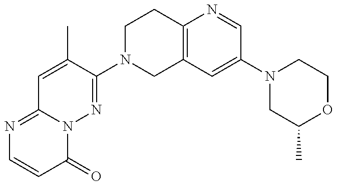 [SMILES: Cc1cc2nccc(=O)n2nc1N1CCc2ncc(N3CCO[C@H](C)C3)cc2C1] | 393.2 |
| 2-3 | 7-(3-(6,7-dihydropyrazolo[1,5- a]pyrimidin-4(5H)-yl)-7,8-dihydro- 1,6-naphthyridin-6(5H)-yl)-9- methyl-4H-pyrimido[1,2- b]pyridazin-4-one | 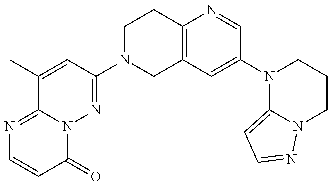 [SMILES: Cc1cc(N2CCc3ncc(N4CCCn5nccc54)cc3C2)nn2c(=O)ccnc12] | 415.2 |
| 2-4 | 3-chloro-7-(3-(6,7- dihydropyrazolo[1,5-a]pyrimidin- 4(5H)-yl)-7,8-dihydro-1,6- naphthyridin-6(5H)-yl)-8-methyl- 4H-pyrimido[1,2-b]pyridazin-4-one | 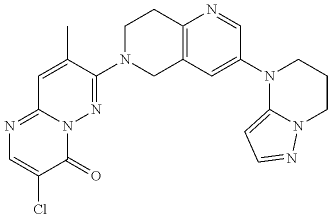 [SMILES: Cc1cc2ncc(Cl)c(=O)n2nc1N1CCc2ncc(N3CCCn4nccc43)cc2C1] | 449.2 |
| 2-5 | 8-methyl-7-(3-(2-methyl-6,7- dihydropyrazolo[1,5-a]pyrimidin- 4(5H)-yl)-7,8-dihydro-1,6- naphthyridin-6(5H)-yl)-4H- pyrimido[1,2-b]pyridazin-4-one | 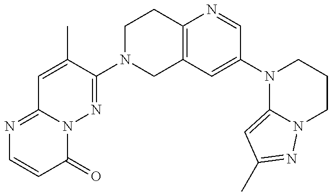 [SMILES: Cc1cc2n(n1)CCCN2c1cnc2c(c1)CN(c1nn3c(=O)ccnc3cc1C)CC2] | 429.4 |
| 2-6 | 7-(3-(1H-pyrazol-1-yl)-7,8- dihydro-1,6-naphthyridin-6(5H)- yl)-8-methyl-4H-pyrimido[1,2- b]pyridazin-4-one | 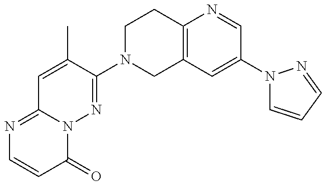 [SMILES: Cc1cc2nccc(=O)n2nc1N1CCc2ncc(-n3cccn3)cc2C1] | 360.4 |
| 2-7 | 7-(3-(2,3-dihydro-1H-pyrido[2,3- b][1,4]oxazin-1-yl)-7,8-dihydro- 1,6-naphthyridin-6(5H)-yl)-8- methyl-4H-pyrimido[1,2- b]pyridazin-4-one | 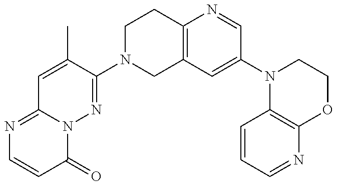 [SMILES: Cc1cc2nccc(=O)n2nc1N1CCc2ncc(N3CCOc4ncccc43)cc2C1] | 428.3 |
| 2-8 | 8-methyl-7-(3-((1-methyl-1H- pyrazol-5-yl)amino)-7,8-dihydro- 1,6-naphthyridin-6(5H)-yl)-4H- pyrimido[1,2-b]pyridazin-4-one | 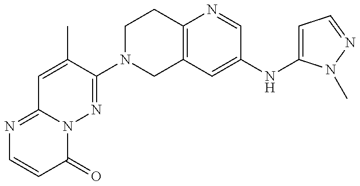 [SMILES: Cc1cc2nccc(=O)n2nc1N1CCc2ncc(Nc3ccnn3C)cc2C1] | 389.4 |
| 2-9 | 7-(3-(6,7-dihydropyrazolo[1,5- a]pyrimidin-4(5H)-yl)-7,8-dihydro- 1,6-naphthyridin-6(5H)-yl)-3- fluoro-2,8-dimethyl-4H- pyrimido[1,2-b]pyridazin-4-one | 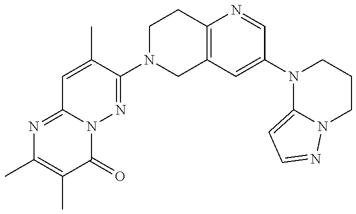 [SMILES: Cc1cc2nc(C)c(C)c(=O)n2nc1N1CCc2ncc(N3CCCn4nccc43)cc2C1] | 447.2 |
| 2-10 | 7-(3-(6,7-dihydropyrazolo[1,5- a]pyrimidin-4(5H)-yl)-7,8-dihydro- 1,6-naphthyridin-6(5H)-yl)-2,8- dimethyl-4H-pyrimido[1,2- b]pyridazin-4-one | 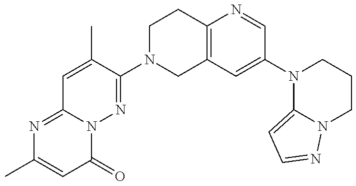 [SMILES: Cc1cc(=O)n2nc(N3CCc4ncc(N5CCCn6nccc65)cc4C3)c(C)cc2n1] | 429.2 |
| 2-11 | 7-(3-(6,7-dihydropyrazolo[1,5- a]pyrimidin-4(5H)-yl)-7,8-dihydro- 1,6-naphthyridin-6(5H)-yl)-8- methyl-2-(trifluoromethyl)-4H- pyrimido[1,2-b]pyridazin-4-one | 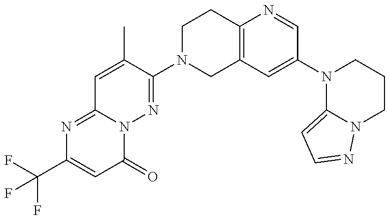 [SMILES: Cc1cc2nc(C(F)(F)F)cc(=O)n2nc1N1CCc2ncc(N3CCCn4nccc43)cc2C1] | 483.2 |
| 2-12 | 7-(3-(6,7-dihydropyrazolo[1,5- a]pyrimidin-4(5H)-yl)-7,8-dihydro- 1,6-naphthyridin-6(5H)-yl)-3- fluoro-2,8,9-trimethyl-4H- pyrimido[1,2-b]pyridazin-4-one | 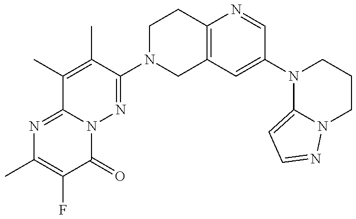 [SMILES: Cc1nc2c(C)c(C)c(N3CCc4ncc(N5CCCn6nccc65)cc4C3)nn2c(=O)c1F] | 461.2 |
| 2-13 | 7-(3-(6,7-dihydropyrazolo[1,5- a]pyrimidin-4(5H)-yl)-7,8-dihydro- 1,6-naphthyridin-6(5H)-yl)-2,3,8- trimethyl-4H-pyrimido[1,2- b]pyridazin-4-one | 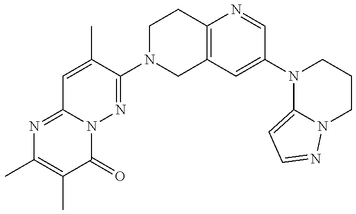 [SMILES: Cc1cc2nc(C)c(C)c(=O)n2nc1N1CCc2ncc(N3CCCn4nccc43)cc2C1] | 443.2 |
| 2-14 | 2-(difluoromethyl)-7-(3-(6,7- dihydropyrazolo[1,5-a]pyrimidin- 4(5H)-yl)-7,8-dihydro-1,6- naphthyridin-6(5H)-yl)-8-methyl- 4H-pyrimido[1,2-b]pyridazin-4-one | 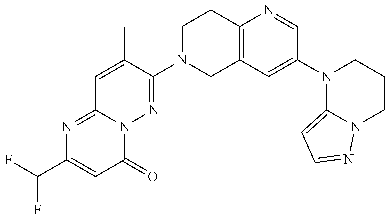 [SMILES: Cc1cc2nc(C(F)F)cc(=O)n2nc1N1CCc2ncc(N3CCCn4nccc43)cc2C1] | 465.2 |
| 2-15 | 2-cyclopropyl-7-(3-(6,7- dihydropyrazolo[1,5-a]pyrimidin- 4(5H)-yl)-7,8-dihydro-1,6- naphthyridin-6(5H)-yl)-8-methyl- 4H-pyrimido[1,2-b]pyridazin-4-one | 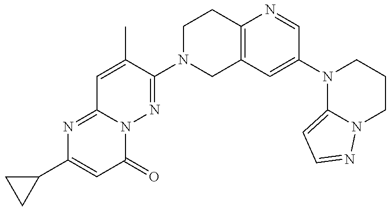 [SMILES: Cc1cc2nc(C3CC3)cc(=O)n2nc1N1CCc2ncc(N3CCCn4nccc43)cc2C1] | 455.2 |
| 2-16 | 3-chloro-7-(3-(6,7- dihydropyrazolo[1,5-a]pyrimidin- 4(5H)-yl)-7,8-dihydro-1,6- naphthyridin-6(5H)-yl)-2,8- dimethyl-4H-pyrimido[1,2- b]pyridazin-4-one | 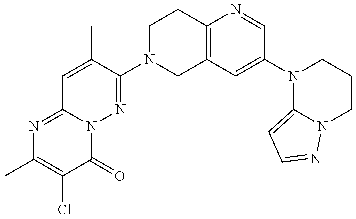 [SMILES: Cc1cc2nc(C)c(Cl)c(=O)n2nc1N1CCc2ncc(N3CCCn4nccc43)cc2C1] | 463.2 |
| 2-17 | 7-(3-(2,3-dihydro-1H-pyrido[2,3- b][1,4]oxazin-1-yl)-7,8-dihydro- 1,6-naphthyridin-6(5H)-yl)-2,8- dimethyl-4H-pyrimido[1,2- b]pyridazin-4-one | 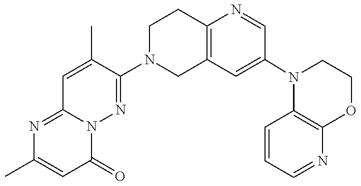 [SMILES: Cc1cc(=O)n2nc(N3CCc4ncc(N5CCOc6ncccc65)cc4C3)c(C)cc2n1] | 442.4 |
| 2-18 | (R)-2,8-dimethyl-7-(3-(2- methylmorpholino)-7,8-dihydro- 1,6-naphthyridin-6(5H)-yl)-4H- pyrimido[1,2-b]pyridazin-4-one | 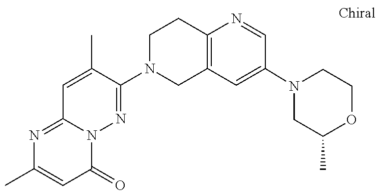 [SMILES: Cc1cc(=O)n2nc(N3CCc4ncc(N5CCO[C@H](C)C5)cc4C3)c(C)cc2n1] | 407.5 |
| 2-19 | 7-(3-((2-fluoropyridin-3-yl)amino)- 7,8-dihydro-1,6-naphthyridin- 6(5H)-yl)-2,8-dimethyl-4H- pyrimido[1,2-b]pyridazin-4-one | 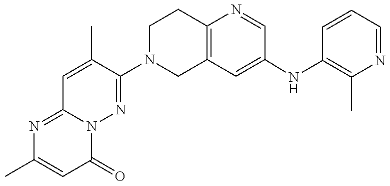 [SMILES: Cc1cc(=O)n2nc(N3CCc4ncc(Nc5cccnc5C)cc4C3)c(C)cc2n1] | 418.2 |
| 2-20 | 2,8-dimethyl-7-(3-((1-methyl-1H- pyrazol-5-yl)amino)-7,8-dihydro- 1,6-naphthyridin-6(5H)-yl)-4H- pyrimido[1,2-b]pyridazin-4-one | 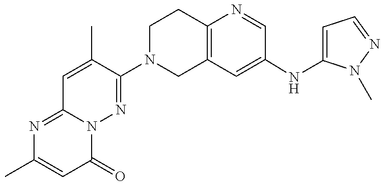 [SMILES: Cc1cc(=O)n2nc(N3CCc4ncc(Nc5ccnn5C)cc4C3)c(C)cc2n1] | 403.2 |
| 2-21 | 2,8-dimethyl-7-(3-(2-methyl-6,7- dihydropyrazolo[1,5-a]pyrimidin- 4(5H)-yl)-7,8-dihydro-1,6- naphthyridin-6(5H)-yl)-4H- pyrimido[1,2-b]pyridazin-4-one | 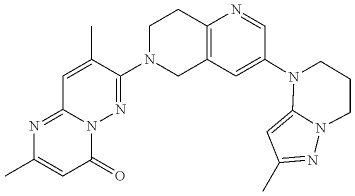 [SMILES: Cc1cc(=O)n2nc(N3CCc4ncc(N5CCCn6nc(C)cc65)cc4C3)c(C)cc2n1] | 443.2 |
| 2-22 | 7-(3-(1H-pyrazol-1-yl)-7,8- dihydro-1,6-naphthyridin-6(5H)- yl)-2,8-dimethyl-4H-pyrimido[1,2- b]pyridazin-4-one | 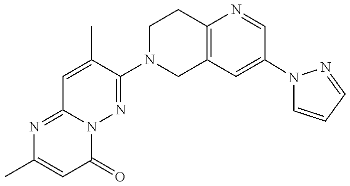 [SMILES: Cc1cc(=O)n2nc(N3CCc4ncc(-n5cccn5)cc4C3)c(C)cc2n1] | 374.2 |
| 2-23 | 7-(3-(6,7-dihydropyrazolo[1,5- a]pyrimidin-4(5H)-yl)-7,8-dihydro- 1,6-naphthyridin-6(5H)-yl)-2,8,9- trimethyl-4H-pyrimido[1,2- b]pyridazin-4-one | 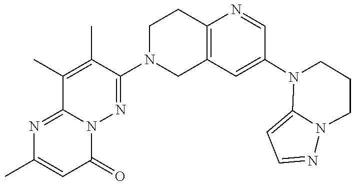 [SMILES: Cc1cc(=O)n2nc(N3CCc4ncc(N5CCCn6nccc65)cc4C3)c(C)c(C)c2n1] | 443.2 |
| 2-24 | 2-(difluoromethyl)-7-(3-(2,2- dimethylmorpholino)-7,8-dihydro- 1,6-naphthyridin-6(5H)-yl)-8- methyl-4H-pyrimido[1,2- b]pyridazin-4-one | 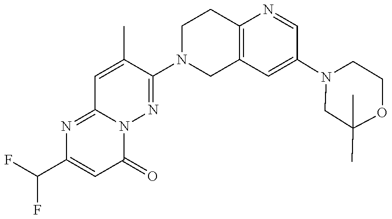 | 457.2 |
| 2-25 | (R)-2-(difluoromethyl)-8-methyl-7- (3-(2-methylmorpholino)-7,8- dihydro-1,6-naphthyridin-6(5H)- yl)-4H-pyrimido[1,2-b]pyridazin-4- one | 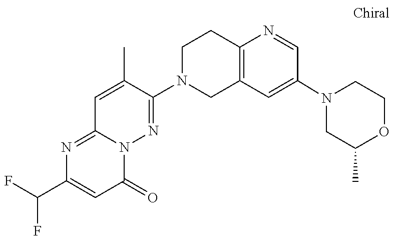 [SMILES: Cc1cc2nc(C(F)F)cc(=O)n2nc1N1CCc2ncc(N3CCO[C@H](C)C3)cc2C1] | 443.2 |
| 2-26 | 7-(3-(1H-pyrazol-1-yl)-7,8- dihydro-1,6-naphthyridin-6(5H)- yl)-2-(difluoromethyl)-8-methyl- 4H-pyrimido[1,2-b]pyridazin-4-one | 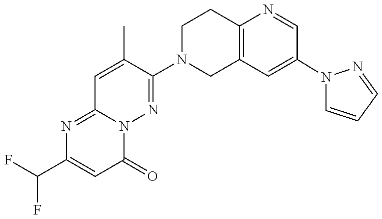 [SMILES: Cc1cc2nc(C(F)F)cc(=O)n2nc1N1CCc2ncc(-n3cccn3)cc2C1] | 410.2 |
| 2-27 | 2-(difluoromethyl)-8-methyl-7-(3- ((1-methyl-1H-pyrazol-5- yl)amino)-7,8-dihydro-1,6- naphthyridin-6(5H)-yl)-4H- pyrimido[1,2-b]pyridazin-4-one | 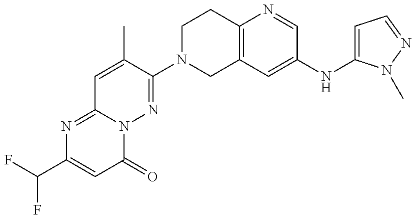 [SMILES: Cc1cc2nc(C(F)F)cc(=O)n2nc1N1CCc2ncc(Nc3ccnn3C)cc2C1] | 439.2 |
| 2-28 | 2-(difluoromethyl)-8-methyl-7-(3- (2-methyl-6,7-dihydropyrazolo[1,5- a]pyrimidin-4(5H)-yl)-7,8-dihydro- 1,6-naphthyridin-6(5H)-yl)-4H- pyrimido[1,2-b]pyridazin-4-one | 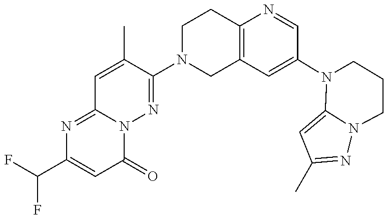 [SMILES: Cc1cc2n(n1)CCCN2c1cnc2c(c1)CN(c1nn3c(=O)cc(C(F)F)nc3cc1C)CC2] | 479.2 |
| 2-29 | 2-(difluoromethyl)-7-(3-(2,3- dihydro-1H-pyrido[2,3- b][1,4]oxazin-1-yl)-7,8-dihydro- 1,6-naphthyridin-6(5H)-yl)-8- methyl-4H-pyrimido[1,2- b]pyridazin-4-one | 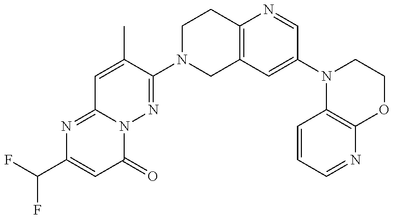 [SMILES: Cc1cc2nc(C(F)F)cc(=O)n2nc1N1CCc2ncc(N3CCOc4ncccc43)cc2C1] | 478.2 |
| 2-30 | 7-(3-(6,7-dihydropyrazolo[1,5- a]pyrimidin-4(5H)-yl)-7,8-dihydro- 1,6-naphthyridin-6(5H)-yl)-8,9- dimethyl-2-(trifluoromethyl)-4H- pyrimido[1,2-b]pyridazin-4-one | 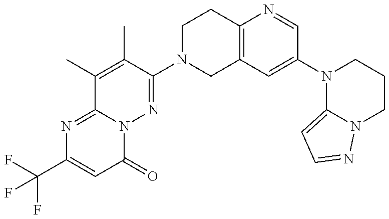 [SMILES: Cc1c(N2CCc3ncc(N4CCCn5nccc54)cc3C2)nn2c(=O)cc(C(F)(F)F)nc2c1C] | 497.2 |
| 2-31 | 2-(difluoromethyl)-7-(3-(6,7- dihydropyrazolo[1,5-a]pyrimidin- 4(5H)-yl)-7,8-dihydro-1,6- naphthyridin-6(5H)-yl)-8,9- dimethyl-4H-pyrimido[1,2- b]pyridazin-4-one | 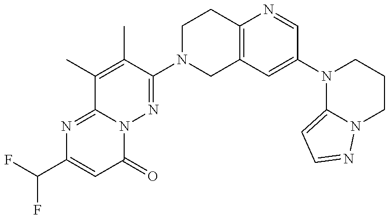 [SMILES: Cc1c(N2CCc3ncc(N4CCCn5nccc54)cc3C2)nn2c(=O)cc(C(F)F)nc2c1C] | 479.2 |
| 2-32 | (R)-8,9-dimethyl-7-(3-(2- methylmorpholino)-7,8-dihydro- 1,6-naphthyridin-6(5H)-yl)-4H- pyrimido[1,2-b]pyridazin-4-one | 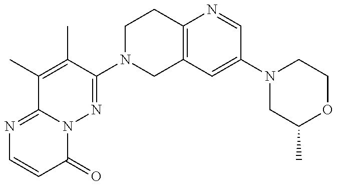 [SMILES: Cc1c(N2CCc3ncc(N4CCO[C@H](C)C4)cc3C2)nn2c(=O)ccnc2c1C] | 407.2 |
| 2-33 | 7-(3-(2,3-dihydro-1H-pyrido[2,3- b][1,4]oxazin-1-yl)-7,8-dihydro- 1,6-naphthyridin-6(5H)-yl)-8,9- dimethyl-4H-pyrimido[1,2- b]pyridazin-4-one | 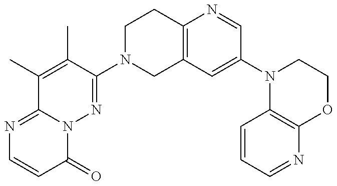 [SMILES: Cc1c(N2CCc3ncc(N4CCOc5ncccc54)cc3C2)nn2c(=O)ccnc2c1C] | 442.2 |
| 2-34 | 7-(3-(1H-pyrazol-1-yl)-7,8- dihydro-1,6-naphthyridin-6(5H)- yl)-8,9-dimethyl-4H-pyrimido[1,2- b]pyridazin-4-one | 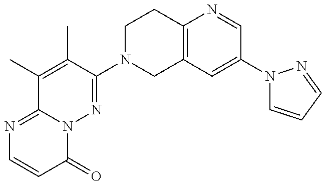 [SMILES: Cc1c(N2CCc3ncc(-n4cccn4)cc3C2)nn2c(=O)ccnc2c1C] | 374.2 |
| 2-35 | 8,9-dimethyl-7-(3-(2-methyl-6,7- dihydropyrazolo[1,5-a]pyrimidin- 4(5H)-yl)-7,8-dihydro-1,6- naphthyridin-6(5H)-yl)-4H- pyrimido[1,2-b]pyridazin-4-one | 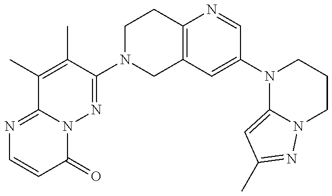 [SMILES: Cc1cc2n(n1)CCCN2c1cnc2c(c1)CN(c1nn3c(=O)ccnc3c(C)c1C)CC2] | 443.4 |
| 2-36 | 7-(3-(6,7-dihydropyrazolo[1,5- a]pyrimidin-4(5H)-yl)-7,8-dihydro- 1,6-naphthyridin-6(5H)-yl)-8,9- dimethyl-4H-pyrimido[1,2- b]pyridazin-4-one | 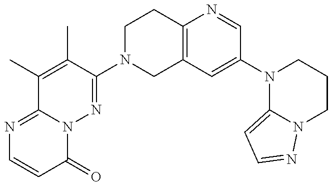 [SMILES: Cc1c(N2CCc3ncc(N4CCCn5nccc54)cc3C2)nn2c(=O)ccnc2c1C] | 456.2 |
| 2-37 | 7-(3-(2-oxa-5- azabicyclo[2.2.1]heptan-5-yl)-7,8- dihydro-1,6-naphthyridin-6(5H)- yl)-2,8,9-trimethyl-4H- pyrimido[1,2-b]pyridazin-4-one | 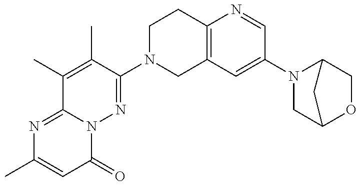 [SMILES: Cc1cc(=O)n2nc(N3CCc4ncc(N5CC6CC5CO6)cc4C3)c(C)c(C)c2n1] | 419.4 |
| 2-38 | 7-(3-((2R,6S)-2,6- dimethylmorpholino)-7,8-dihydro- 1,6-naphthyridin-6(5H)-yl)-2,8,9- trimethyl-4H-pyrimido[1,2- b]pyridazin-4-one | 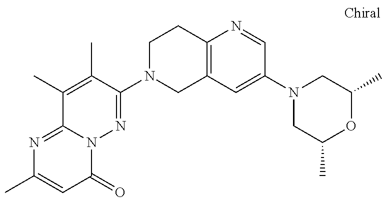 | 435.4 |
| 2-39 | 7-(3-(2,2-dimethylmorpholino)-7,8- dihydro-1,6-naphthyridin-6(5H)- yl)-2,8,9-trimethyl-4H- pyrimido[1,2-b]pyridazin-4-one | 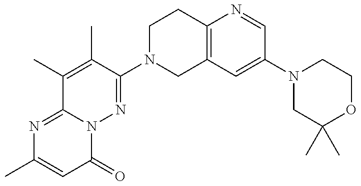 [SMILES: Cc1cc(=O)n2nc(N3CCc4ncc(N5CCOC(C)(C)C5)cc4C3)c(C)c(C)c2n1] | 435.3 |
| 2-40 | 2-(difluoromethyl)-7-(3-(2,2- dimethylmorpholino)-7,8-dihydro- 1,6-naphthyridin-6(5H)-yl)-8,9- dimethyl-4H-pyrimido[1,2- b]pyridazin-4-one | 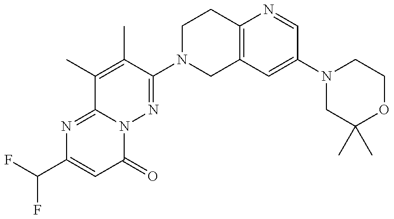 [SMILES: Cc1c(N2CCc3ncc(N4CCOC(C)(C)C4)cc3C2)nn2c(=O)cc(C(F)F)nc2c1C] | 471.4 |
| 2-41 | 2-(difluoromethyl)-7-(3-((2R,6S)- 2,6-dimethylmorpholino)-7,8- dihydro-1,6-naphthyridin-6(5H)- yl)-8,9-dimethyl-4H-pyrimido[1,2- b]pyridazin-4-one | 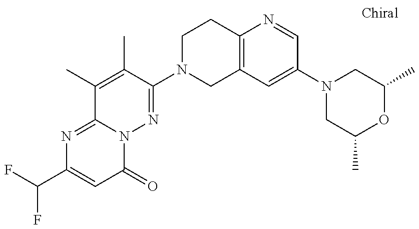 [SMILES: Cc1c(N2CCc3ncc(N4C[C@@H](C)O[C@@H](C)C4)cc3C2)nn2c(=O)cc(C(F)F)nc2c1C] | 471.4 |
| 2-42 | 7-(3-(2-oxa-5- azabicyclo[2.2.1]heptan-5-yl)-7,8- dihydro-1,6-naphthyridin-6(5H)- yl)-2,8-dimethyl-4H-pyrimido[1,2- b]pyridazin-4-one | 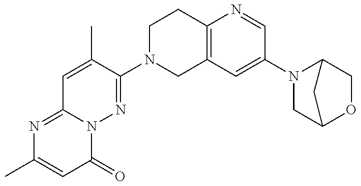 [SMILES: Cc1cc(=O)n2nc(N3CCc4ncc(N5CC6CC5CO6)cc4C3)c(C)cc2n1] | 405.3 |
| 2-43 | 7-(3-(8-oxa-3- azabicyclo[3.2.1]octan-3-yl)-7,8- dihydro-1,6-naphthyridin-6(5H)- yl)-2,8,9-trimethyl-4H- pyrimido[1,2-b]pyridazin-4-one | 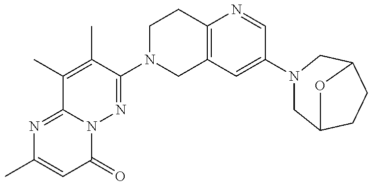 [SMILES: Cc1cc(=O)n2nc(N3CCc4ncc(N5CC6CCC(C5)O6)cc4C3)c(C)c(C)c2n1] | 433.4 |
| 2-44 | 2,8,9-trimethyl-7-(3-(((tetrahydro- 2H-pyran-3-yl)methyl)amino)-7,8- dihydro-1,6-naphthyridin-6(5H)- yl)-4H-pyrimido[1,2-b]pyridazin-4- one | 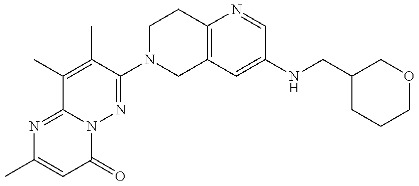 [SMILES: Cc1cc(=O)n2nc(N3CCc4ncc(NCC5CCCOC5)cc4C3)c(C)c(C)c2n1] | 435.3 |
| 2-45 | 7-(3-(4-methoxypiperidin-1-yl)-7,8- dihydro-1,6-naphthyridin-6(5H)- yl)-2,8,9-trimethyl-4H- pyrimido[1,2-b]pyridazin-4-one | 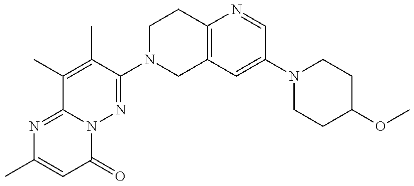 | 435.4 |
| 2-46 | 2,8,9-trimethyl-7-(3- thiomorpholino-7,8-dihydro-1,6- naphthyridin-6(5H)-yl)-4H- pyrimido[1,2-b]pyridazin-4-one | 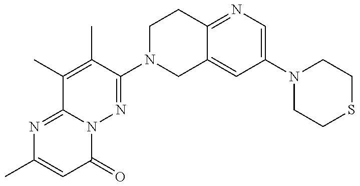 [SMILES: Cc1cc(=O)n2nc(N3CCc4ncc(N5CCSCC5)cc4C3)c(C)c(C)c2n1] | 423.4 |
| 2-47 | 7-(3-(2-oxa-7-azaspiro[3.5]nonan- 7-yl)-7,8-dihydro-1,6-naphthyridin- 6(5H)-yl)-2,8,9-trimethyl-4H- pyrimido[1,2-b]pyridazin-4-one | 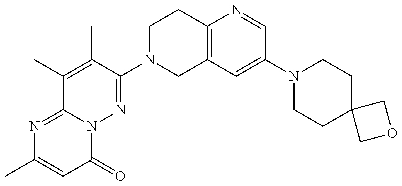 [SMILES: Cc1cc(=O)n2nc(N3CCc4ncc(N5CCC6(CC5)COC6)cc4C3)c(C)c(C)c2n1] | 447.5 |
| 2-48 | 2-(difluoromethyl)-7-(3-(4,4- difluoropiperidin-1-yl)-7,8-dihydro- 1,6-naphthyridin-6(5H)-yl)-8,9- dimethyl-4H-pyrimido[1,2- b]pyridazin-4-one | 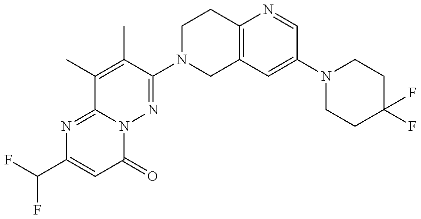 [SMILES: Cc1c(N2CCc3ncc(N4CCC(F)(F)CC4)cc3C2)nn2c(=O)cc(C(F)F)nc2c1C] | 477.4 |
| 2-49 | 2,8,9-trimethyl-7-(3-(2-(pyridin-3- yl)morpholino)-7,8-dihydro-1,6- naphthyridin-6(5H)-yl)-4H- pyrimido[1,2-b]pyridazin-4-one | 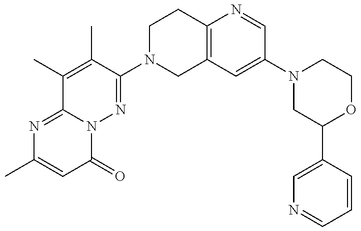 [SMILES: Cc1cc(=O)n2nc(N3CCc4ncc(N5CCOC(c6cccnc6)C5)cc4C3)c(C)c(C)c2n1] | 484.2 |
| 2-50 | 2,8,9-trimethyl-7-(3-(((2- methylthiazol-4-yl)methyl)amino)- 7,8-dihydro-1,6-naphthyridin- 6(5H)-yl)-4H-pyrimido[1,2- b]pyridazin-4-one | 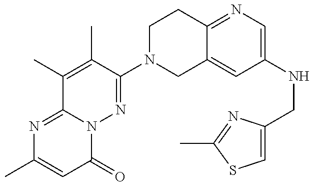 [SMILES: Cc1cc(=O)n2nc(N3CCc4ncc(NCc5csc(C)n5)cc4C3)c(C)c(C)c2n1] | 448.2 |
| 2-51 | 7-(3-(2,2-dimethyl-2,3-dihydro-4H- benzo[b][1,4]oxazin-4-yl)-7,8- dihydro-1,6-naphthyridin-6(5H)- yl)-8-methyl-4H-pyrimido[1,2- b]pyridazin-4-one | 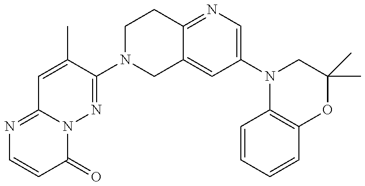 [SMILES: Cc1cc2nccc(=O)n2nc1N1CCc2ncc(N3CC(C)(C)Oc4ccccc43)cc2C1] | 455 |
| 2-52 | 7-(3-(7-fluoro-3-methyl-2,3- dihydro-4H-benzo[b][1,4]oxazin-4- yl)-7,8-dihydro-1,6-naphthyridin- 6(5H)-yl)-8-methyl-4H- pyrimido[1,2-b]pyridazin-4-one | 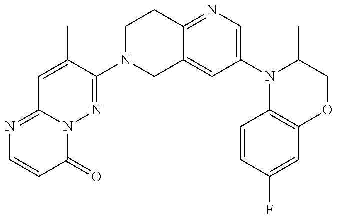 [SMILES: Cc1cc2nccc(=O)n2nc1N1CCc2ncc(N3c4ccc(F)cc4OCC3C)cc2C1] | 459 |
| 2-53 | 8-methyl-7-(3-(6-methyl-2,3- dihydro-4H-benzo[b][1,4]oxazin-4- yl)-7,8-dihydro-1,6-naphthyridin- 6(5H)-yl)-4H-pyrimido[1,2- b]pyridazin-4-one | 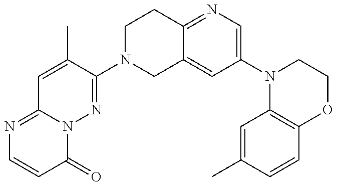 [SMILES: Cc1ccc2c(c1)N(c1cnc3c(c1)CN(c1nn4c(=O)ccnc4cc1C)CC3)CCO2] | 441 |
| 2-54 | 7-(3-(3,6-dimethyl-2,3-dihydro-4H- benzo[b][1,4]oxazin-4-yl)-7,8- dihydro-1,6-naphthyridin-6(5H)- yl)-8-methyl-4H-pyrimido[1,2- b]pyridazin-4-one | 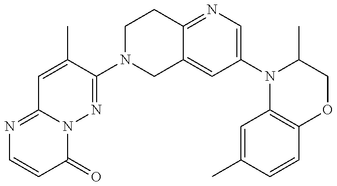 [SMILES: Cc1ccc2c(c1)N(c1cnc3c(c1)CN(c1nn4c(=O)ccnc4cc1C)CC3)C(C)CO2] | 455 |
| 2-55 | 7-(3-(7-fluoro-2-methyl-2,3- dihydro-4H-benzo[b][1,4]oxazin-4- yl)-7,8-dihydro-1,6-naphthyridin- 6(5H)-yl)-8-methyl-4H- pyrimido[1,2-b]pyridazin-4-one | 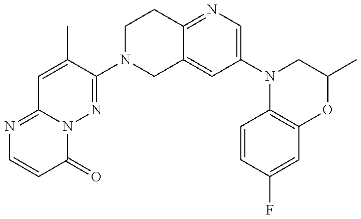 [SMILES: Cc1cc2nccc(=O)n2nc1N1CCc2ncc(N3CC(C)Oc4cc(F)ccc43)cc2C1] | 459 |
| 2-56 | 7-(3-(7-fluoro-2,3-dihydro-4H- benzo[b][1,4]oxazin-4-yl)-7,8- dihydro-1,6-naphthyridin-6(5H)- yl)-8-methyl-4H-pyrimido[1,2- b]pyridazin-4-one | 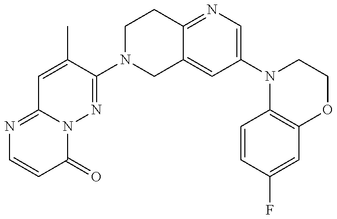 [SMILES: Cc1cc2nccc(=O)n2nc1N1CCc2ncc(N3CCOc4cc(F)ccc43)cc2C1] | 445 |
| 2-57 | 8-methyl-7-(3-(2-methyl-2,3- dihydro-4H-benzo[b][1,4]oxazin-4- yl)-7,8-dihydro-1,6-naphthyridin- 6(5H)-yl)-4H-pyrimido[1,2- b]pyridazin-4-one | 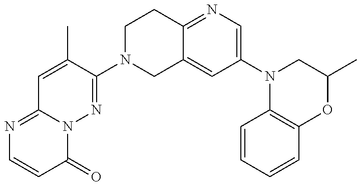 [SMILES: Cc1cc2nccc(=O)n2nc1N1CCc2ncc(N3CC(C)Oc4ccccc43)cc2C1] | 441 |
| 2-58 | 7-(3-(2,3-dihydro-1H-pyrrolo[3,2- b]pyridin-1-yl)-7,8-dihydro-1,6- naphthyridin-6(5H)-yl)-8-methyl- 4H-pyrimido[1,2-b]pyridazin-4-one | 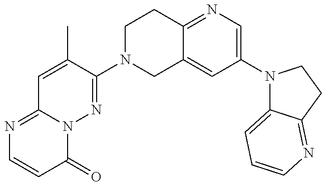 [SMILES: Cc1cc2nccc(=O)n2nc1N1CCc2ncc(N3CCc4ncccc43)cc2C1] | 412 |
| 2-59 | 7-(3-(2-(2- fluorophenyl)morpholino)-7,8- dihydro-1,6-naphthyridin-6(5H)- yl)-8-methyl-4H-pyrimido[1,2- b]pyridazin-4-one | 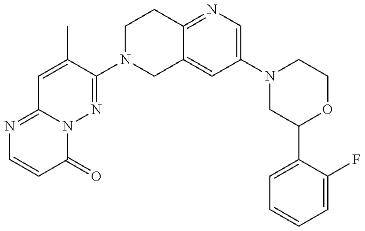 [SMILES: Cc1cc2nccc(=O)n2nc1N1CCc2ncc(N3CCOC(c4ccccc4F)C3)cc2C1] | 473 |
| 2-60 | 8-methyl-7-(3-(phenylamino)-7,8- dihydro-1,6-naphthyridin-6(5H)- yl)-4H-pyrimido[1,2-b]pyridazin-4- one | 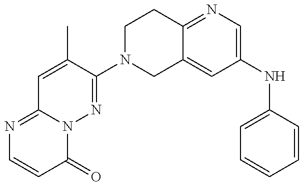 [SMILES: Cc1cc2nccc(=O)n2nc1N1CCc2ncc(Nc3ccccc3)cc2C1] | 385 |
| 2-61 | 7-(3-(2-(3- fluorophenyl)morpholino)-7,8- dihydro-1,6-naphthyridin-6(5H)- yl)-8-methyl-4H-pyrimido[1,2- b]pyridazin-4-one | 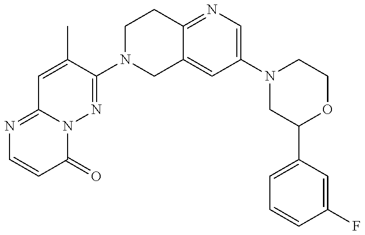 [SMILES: Cc1cc2nccc(=O)n2nc1N1CCc2ncc(N3CCOC(c4cccc(F)c4)C3)cc2C1] | 473 |
| 2-62 | 7-(3-((2-methoxyphenyl)amino)- 7,8-dihydro-1,6-naphthyridin- 6(5H)-yl)-8-methyl-4H- pyrimido[1,2-b]pyridazin-4-one | 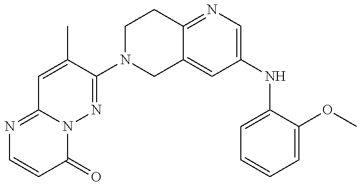 [SMILES: COc1ccccc1Nc1cnc2c(c1)CN(c1nn3c(=O)ccnc3cc1C)CC2] | 415 |
| 2-63 | 7-(3-((4-fluorophenyl)amino)-7,8- dihydro-1,6-naphthyridin-6(5H)- yl)-8-methyl-4H-pyrimido[1,2- b]pyridazin-4-one | 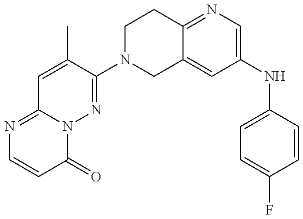 [SMILES: Cc1cc2nccc(=O)n2nc1N1CCc2ncc(Nc3ccc(F)cc3)cc2C1] | 403 |
| 2-64 | 7-(3-(2-(4- fluorophenyl)morpholino)-7,8- dihydro-1,6-naphthyridin-6(5H)- yl)-8-methyl-4H-pyrimido[1,2- b]pyridazin-4-one | 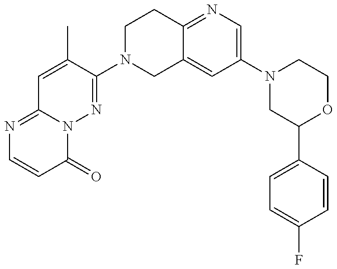 [SMILES: Cc1cc2nccc(=O)n2nc1N1CCc2ncc(N3CCOC(c4ccc(F)cc4)C3)cc2C1] | 473 |
| 2-65 | 7-(3-((4-fluoro-2- methylphenyl)amino)-7,8-dihydro- 1,6-naphthyridin-6(5H)-yl)-8- methyl-4H-pyrimido[1,2- b]pyridazin-4-one | 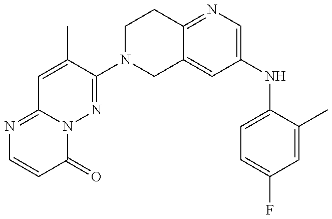 [SMILES: Cc1cc(F)ccc1Nc1cnc2c(c1)CN(c1nn3c(=O)ccnc3cc1C)CC2] | 417 |
| 2-66 | 8-methyl-7-(3-(2- phenylmorpholino)-7,8-dihydro- 1,6-naphthyridin-6(5H)-yl)-4H- pyrimido[1,2-b]pyridazin-4-one | 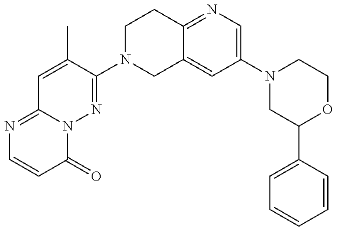 [SMILES: Cc1cc2nccc(=O)n2nc1N1CCc2ncc(N3CCOC(c4ccccc4)C3)cc2C1] | 455 |
| 2-67 | 7-(3-((2-chlorophenyl)amino)-7,8- dihydro-1,6-naphthyridin-6(5H)- yl)-8-methyl-4H-pyrimido[1,2- b]pyridazin-4-one | 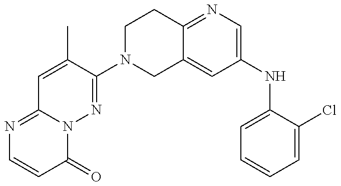 [SMILES: Cc1cc2nccc(=O)n2nc1N1CCc2ncc(Nc3ccccc3Cl)cc2C1] | 419 |
| 2-68 | 7-(3-(2,2-dimethylmorpholino)-7,8- dihydro-1,6-naphthyridin-6(5H)- yl)-8-methyl-4H-pyrimido[1,2- b]pyridazin-4-one | 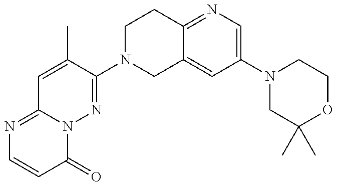 [SMILES: Cc1cc2nccc(=O)n2nc1N1CCc2ncc(N3CCOC(C)(C)C3)cc2C1] | 407 |
| 2-69 | 7-(3-((5-fluoro-2- methylphenyl)amino)-7,8-dihydro- 1,6-naphthyridin-6(5H)-yl)-8- methyl-4H-pyrimido[1,2- b]pyridazin-4-one | 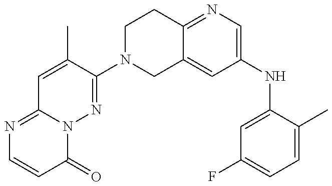 [SMILES: Cc1ccc(F)cc1Nc1cnc2c(c1)CN(c1nn3c(=O)ccnc3cc1C)CC2] | 417 |
| 2-70 | 7-(3-((2-fluoro-5- methoxyphenyl)amino)-7,8- dihydro-1,6-naphthyridin-6(5/)- yl)-8-methyl-4H-pyrimido[1,2- b]pyridazin-4-one | 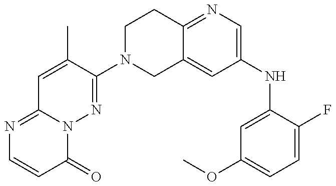 [SMILES: COc1ccc(F)c(Nc2cnc3c(c2)CN(c2nn4c(=O)ccnc4cc2C)CC3)c1] | 433 |
| 2-71 | 7-(3-((3- (difluoromethoxy)phenyl)amino)- 7,8-dihydro-1,6-naphthyridin- 6(5H)-yl)-8-methyl-4H- pyrimido[1,2-b]pyridazin-4-one | 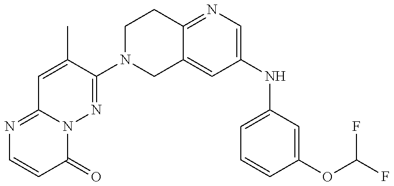 [SMILES: Cc1cc2nccc(=O)n2nc1N1CCc2ncc(Nc3cccc(OC(F)F)c3)cc2C1] | 451 |
| 2-72 | 7-(3-((2,3- dihydrobenzo[b][1,4]dioxin-5- yl)amino)-7,8-dihydro-1,6- naphthyridin-6(5H)-yl)-8-methyl- 4H-pyrimido[1,2-b]pyridazin-4-one | 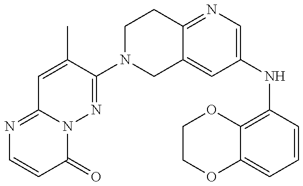 [SMILES: Cc1cc2nccc(=O)n2nc1N1CCc2ncc(Nc3cccc4c3OCCO4)cc2C1] | 443 |
| 2-73 | (S)-8-methyl-7-(3-(2- phenylmorpholino)-7,8-dihydro- 1,6-naphthyridin-6(5H)-yl)-4H- pyrimido[1,2-b]pyridazin-4-one | 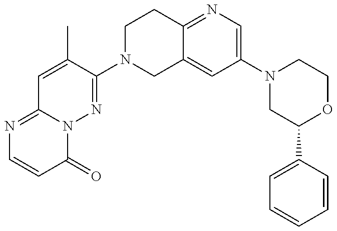 [SMILES: Cc1cc2nccc(=O)n2nc1N1CCc2ncc(N3CCO[C@H](c4ccccc4)C3)cc2C1] | 455 |
| 2-74 | 7-(3-((4-fluoro-2- methoxyphenyl)amino)-7,8- dihydro-1,6-naphthyridin-6(5H)- yl)-8-methyl-4H-pyrimido[1,2- b]pyridazin-4-one | 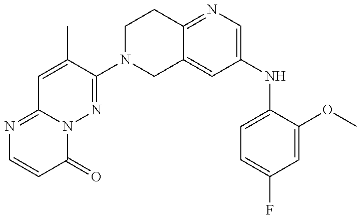 [SMILES: COc1cc(F)ccc1Nc1cnc2c(c1)CN(c1nn3c(=O)ccnc3cc1C)CC2] | 433 |
| 2-75 | 8-methyl-7-(3-(o-tolylamino)-7,8- dihydro-1,6-naphthyridin-6(5H)- yl)-4H-pyrimido[1,2-b]pyridazin-4- one | 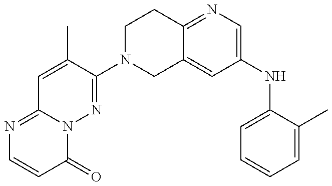 [SMILES: Cc1ccccc1Nc1cnc2c(c1)CN(c1nn3c(=O)ccnc3cc1C)CC2] | 399 |
| 2-76 | 8-methyl-7-(3-(2-(pyridin-2- yl)morpholino)-7,8-dihydro-1,6- naphthyridin-6(5H)-yl)-4H- pyrimido[1,2-b]pyridazin-4-one | 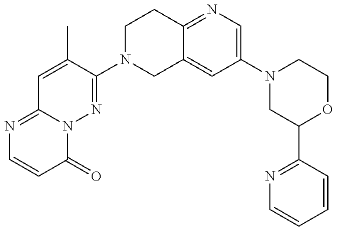 [SMILES: Cc1cc2nccc(=O)n2nc1N1CCc2ncc(N3CCOC(c4ccccn4)C3)cc2C1] | 456 |
| 2-77 | 8-methyl-7-(3-(2-(pyridin-3- yl)morpholino)-7,8-dihydro-1,6- naphthyridin-6(5H)-yl)-4H- pyrimido[1,2-b]pyridazin-4-one | 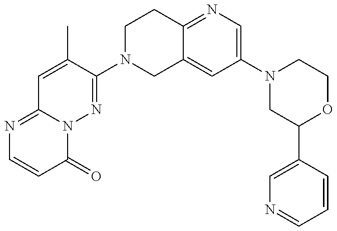 [SMILES: Cc1cc2nccc(=O)n2nc1N1CCc2ncc(N3CCOC(c4cccnc4)C3)cc2C1] | 456 |
| 2-78 | 7-(3-((2-fluoropyridin-3-yl)amino)- 7,8-dihydro-1,6-naphthyridin- 6(5H)-yl)-8-methyl-4H- pyrimido[1,2-b]pyridazin-4-one | 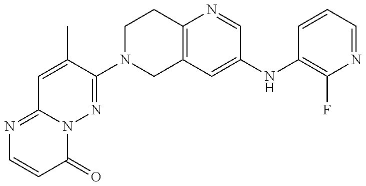 [SMILES: Cc1cc2nccc(=O)n2nc1N1CCc2ncc(Nc3cccnc3F)cc2C1] | 404 |
| 2-79 | 7-(3-((3-fluorophenyl)amino)-7,8- dihydro-1,6-naphthyridin-6(5H)- yl)-8-methyl-4H-pyrimido[1,2- b]pyridazin-4-one | 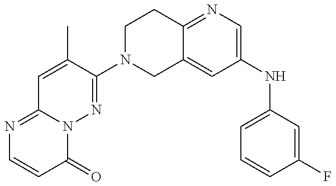 [SMILES: Cc1cc2nccc(=O)n2nc1N1CCc2ncc(Nc3cccc(F)c3)cc2C1] | 403 |
| 2-80 | 3-((6-(8-methyl-4-oxo-4H- pyrimido[1,2-b]pyridazin-7-yl)- 5,6,7,8-tetrahydro-1,6- naphthyridin-3- yl)amino)benzonitrile | 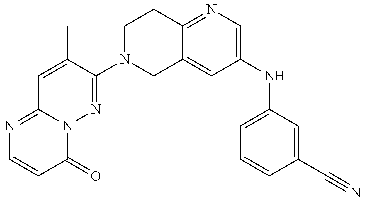 [SMILES: Cc1cc2nccc(=O)n2nc1N1CCc2ncc(Nc3cccc(C#N)c3)cc2C1] | 410 |
| 2-81 | 4-fluoro-2-((6-(8-methyl-4-oxo-4H- pyrimido[1,2-b]pyridazin-7-yl)- 5,6,7,8-tetrahydro-1,6- naphthyridin-3- yl)amino)benzonitrile | 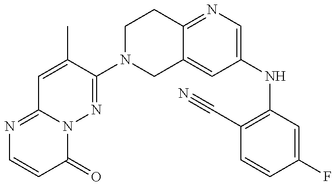 [SMILES: Cc1cc2nccc(=O)n2nc1N1CCc2ncc(Nc3cc(F)ccc3C#N)cc2C1] | 428 |
| 2-82 | 7-(3-((3-chloro-5- methoxyphenyl)amino)-7,8- dihydro-1,6-naphthyridin-6(5H)- yl)-8-methyl-4H-pyrimido[1,2- b]pyridazin-4-one | 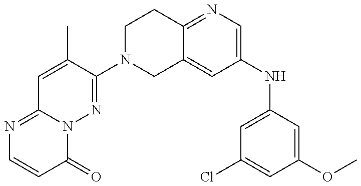 [SMILES: COc1cc(Cl)cc(Nc2cnc3c(c2)CN(c2nn4c(=O)ccnc4cc2C)CC3)c1] | 449 |
| 2-83 | 8-methyl-7-(3-((3-(2-methylthiazol- 4-yl)phenyl)amino)-7,8-dihydro- 1,6-naphthyridin-6(5H)-yl)-4H- pyrimido[1,2-b]pyridazin-4-one | 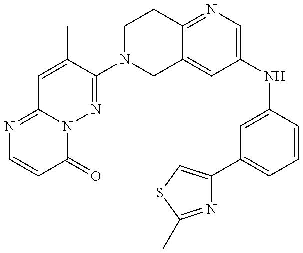 [SMILES: Cc1nc(-c2cccc(Nc3cnc4c(c3)CN(c3nn5c(=O)ccnc5cc3C)CC4)c2)cs1] | 482 |
| 2-84 | 7-(3-(2-(3- methoxyphenyl)morpholino)-7,8- dihydro-1,6-naphthyridin-6(5H)- yl)-8-methyl-4H-pyrimido[1,2- b]pyridazin-4-one | 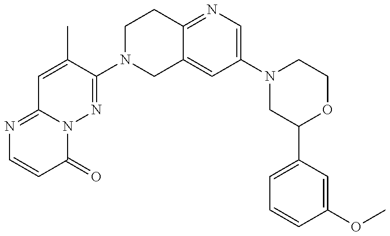 [SMILES: COc1cccc(C2CN(c3cnc4c(c3)CN(c3nn5c(=O)ccnc5cc3C)CC4)CCO2)c1] | 485 |
| 2-85 | 7-(3-(indolin-1-yl)-7,8-dihydro-1,6- naphthyridin-6(5H)-yl)-8-methyl- 4H-pyrimido[1,2-b]pyridazin-4-one | 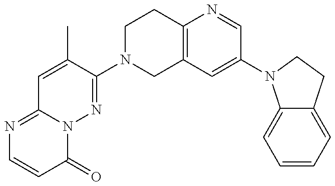 [SMILES: Cc1cc2nccc(=O)n2nc1N1CCc2ncc(N3CCc4ccccc43)cc2C1] | 411 |
| 2-86 | 8-methyl-7-(3-(((tetrahydro-2H- pyran-3-yl)methyl)amino)-7,8- dihydro-1,6-naphthyridin-6(5H)- yl)-4H-pyrimido[1,2-b]pyridazin-4- one | 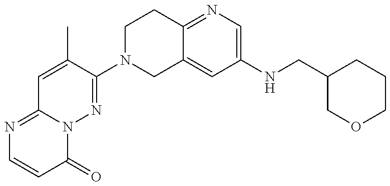 [SMILES: Cc1cc2nccc(=O)n2nc1N1CCc2ncc(NCC3CCCOC3)cc2C1] | 407 |
| 2-87 | 7-(3-((6-fluoropyridin-3-yl)amino)- 7,8-dihydro-1,6-naphthyridin- 6(5H)-yl)-8-methyl-4H- pyrimido[1,2-b]pyridazin-4-one | 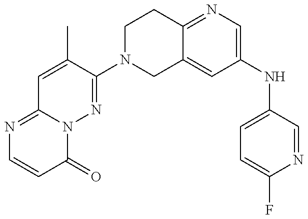 [SMILES: Cc1cc2nccc(=O)n2nc1N1CCc2ncc(Nc3ccc(F)nc3)cc2C1] | 404 |
| 2-88 | 8-methyl-7-(3-(pyridin-3-ylamino)- 7,8-dihydro-1,6-naphthyridin- 6(5H)-yl)-4H-pyrimido[1,2- b]pyridazin-4-one | 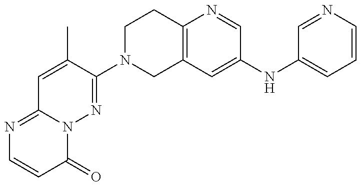 [SMILES: Cc1cc2nccc(=O)n2nc1N1CCc2ncc(Nc3cccnc3)cc2C1] | 386 |
| 2-89 | 7-(3-((cyclopropylmethyl)amino)- 7,8-dihydro-1,6-naphthyridin- 6(5H)-yl)-8-methyl-4H- pyrimido[1,2-b]pyridazin-4-one | 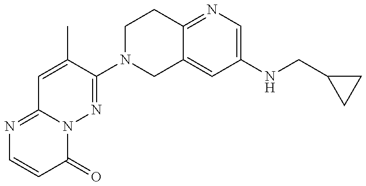 [SMILES: Cc1cc2nccc(=O)n2nc1N1CCc2ncc(NCC3CC3)cc2C1] | 363 |
| 2-90 | 7-(3-(isobutylamino)-7,8-dihydro- 1,6-naphthyridin-6(5H)-yl)-8- methyl-4H-pyrimido[1,2- b]pyridazin-4-one | 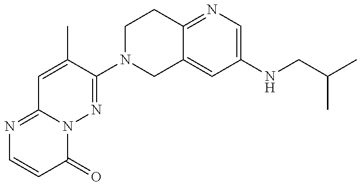 [SMILES: Cc1cc2nccc(=O)n2nc1N1CCc2ncc(NCC(C)C)cc2C1] | 365 |
| 2-91 | 8-methyl-7-(3 -(((tetrahydro-2H- pyran-4-yl)methyl)amino)-7,8- dihydro-1,6-naphthyridin-6(5H)- yl)-4H-pyrimido[1,2-b]pyridazin-4- one | 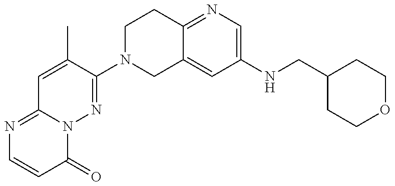 [SMILES: Cc1cc2nccc(=O)n2nc1N1CCc2ncc(NCC3CCOCC3)cc2C1] | 407 |
| 2-92 | 7-(3-(diethylamino)-7,8-dihydro- 1,6-naphthyridin-6(5H)-yl)-8- methyl-4H-pyrimido[1,2- b]pyridazin-4-one | 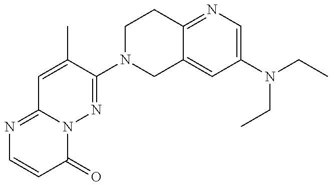 [SMILES: CCN(CC)c1cnc2c(c1)CN(c1nn3c(=O)ccnc3cc1C)CC2] | 365 |
| 2-93 | 8-methyl-7-(3 -(2-(pyridin-4- yl)morpholino)-7,8-dihydro-1,6- naphthyridin-6(5H)-yl)-4H- pyrimido[1,2-b]pyridazin-4-one | 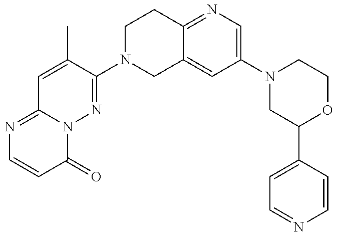 [SMILES: Cc1cc2nccc(=O)n2nc1N1CCc2ncc(N3CCOC(c4ccncc4)C3)cc2C1] | 456 |
| 2-94 | 7-(3-((3-fluoro-2- methylphenyl)amino)-7,8-dihydro- 1,6-naphthyridin-6(5H)-yl)-8- methyl-4H-pyrimido[1,2- b]pyridazin-4-one | 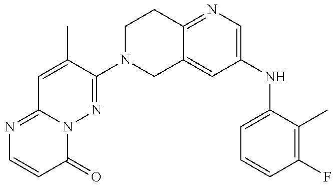 [SMILES: Cc1cc2nccc(=O)n2nc1N1CCc2ncc(Nc3cccc(F)c3C)cc2C1] | 417 |
| 2-95 | 7-(3-(2-(4- methoxyphenyl)morpholino)-7,8- dihydro-1,6-naphthyridin-6(5H)- yl)-8-methyl-4H-pyrimido[1,2- b]pyridazin-4-one | 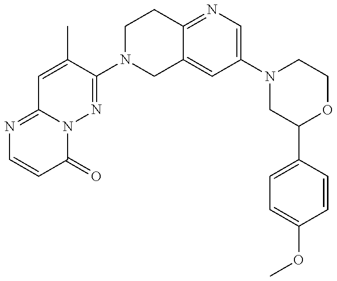 [SMILES: COc1ccc(C2CN(c3cnc4c(c3)CN(c3nn5c(=O)ccnc5cc3C)CC4)CCO2)cc1] | 485 |
| 2-96 | (R)-8-methyl-7-(3-(2- phenylmorpholino)-7,8-dihydro- 1,6-naphthyridin-6(5H)-yl)-4H- pyrimido[1,2-b]pyridazin-4-one | 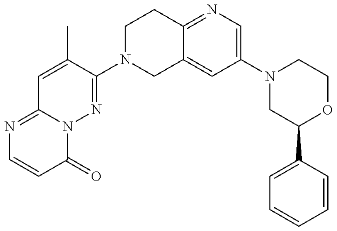 [SMILES: Cc1cc2nccc(=O)n2nc1N1CCc2ncc(N3CCO[C@@H](c4ccccc4)C3)cc2C1] | 455 |
| 2-97 | 8-methyl-7-(3-(pyridin-4-ylamino)- 7,8-dihydro-1,6-naphthyridin- 6(5H)-yl)-4H-pyrimido[1,2- b]pyridazin-4-one | 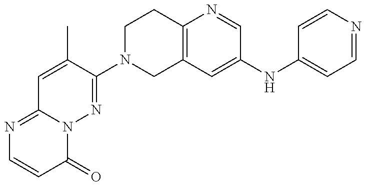 [SMILES: Cc1cc2nccc(=O)n2nc1N1CCc2ncc(Nc3ccncc3)cc2C1] | 386 |
| 2-98 | 7-(3-((2-fluorophenyl)amino)-7,8- dihydro-1,6-naphthyridin-6(5H)- yl)-8-methyl-4H-pyrimido[1,2- b]pyridazin-4-one | 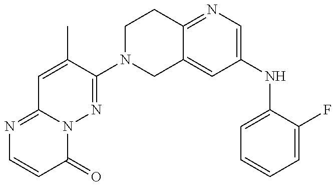 [SMILES: Cc1cc2nccc(=O)n2nc1N1CCc2ncc(Nc3ccccc3F)cc2C1] | 403 |
| 2-99 | 7-(3-(2-benzylmorpholino)-7,8- dihydro-1,6-naphthyridin-6(5H)- yl)-8-methyl-4H-pyrimido[1,2- b]pyridazin-4-one | 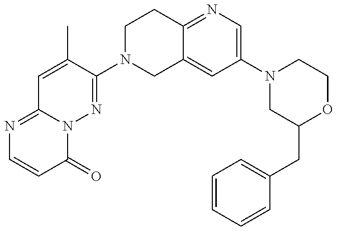 [SMILES: Cc1cc2nccc(=O)n2nc1N1CCc2ncc(N3CCOC(Cc4ccccc4)C3)cc2C1] | 469 |
| 2-100 | 7-(3-((2,6-difluoropyridin-4- yl)amino)-7,8-dihydro-1,6- naphthyridin-6(5H)-yl)-8-methyl- 4H-pyrimido[1,2-b]pyridazin-4-one | 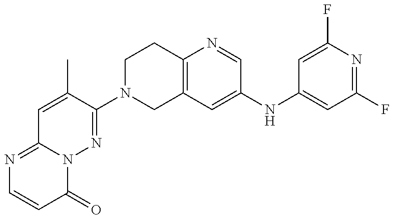 [SMILES: Cc1cc2nccc(=O)n2nc1N1CCc2ncc(Nc3cc(F)nc(F)c3)cc2C1] | 422 |
| 2-101 | 7-(3-((6-isopropylpyridin-3- yl)amino)-7,8-dihydro-1,6- naphthyridin-6(5H)-yl)-8-methyl- 4H-pyrimido[1,2-b]pyridazin-4-one | 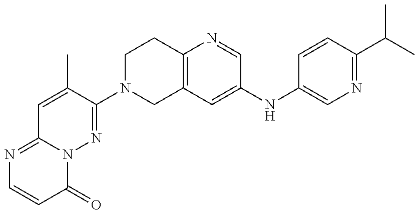 [SMILES: Cc1cc2nccc(=O)n2nc1N1CCc2ncc(Nc3ccc(C(C)C)nc3)cc2C1] | 428 |
| 2-102 | 8-methyl-7-(3-((3-methylpyridin-4- yl)amino)-7,8-dihydro-1,6- naphthyridin-6(5H)-yl)-4H- pyrimido[1,2-b]pyridazin-4-one | 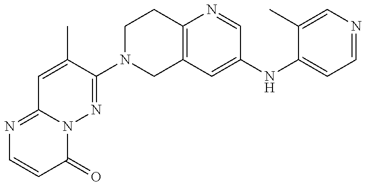 [SMILES: Cc1cnccc1Nc1cnc2c(c1)CN(c1nn3c(=O)ccnc3cc1C)CC2] | 400 |
| 2-103 | 7-(3-(6-fluoro-3,4-dihydroquinolin- 1(2H)-yl)-7,8-dihydro-1,6- naphthyridin-6(5H)-yl)-8-methyl- 4H-pyrimido[1,2-b]pyridazin-4-one | 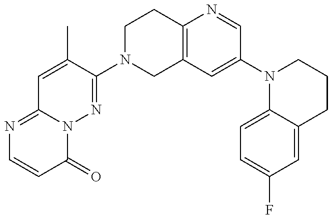 [SMILES: Cc1cc2nccc(=O)n2nc1N1CCc2ncc(N3CCCc4cc(F)ccc43)cc2C1] | 443 |
| 2-104 | 3-fluoro-5-((6-(8-methyl-4-oxo-4H- pyrimido[1,2-b]pyridazin-7-yl)- 5,6,7,8-tetrahydro-1,6- naphthyridin-3- yl)amino)benzonitrile | 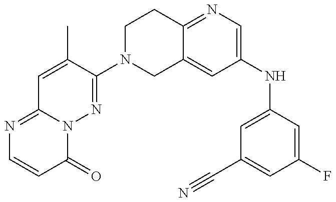 [SMILES: Cc1cc2nccc(=O)n2nc1N1CCc2ncc(Nc3cc(F)cc(C#N)c3)cc2C1] | 428 |
| 2-105 | 7-(3-((3,5-difluorophenyl)amino)- 7,8-dihydro-1,6-naphthyridin- 6(5H)-yl)-8-methyl-4H- pyrimido[1,2-b]pyridazin-4-one | 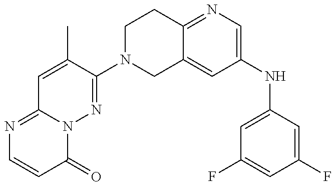 [SMILES: Cc1cc2nccc(=O)n2nc1N1CCc2ncc(Nc3cc(F)cc(F)c3)cc2C1] | 421 |
| 2-106 | 7-(3-((2,3-difluorophenyl)amino)- 7,8-dihydro-1,6-naphthyridin- 6(5H)-yl)-8-methyl-4H- pyrimido[1,2-b]pyridazin-4-one | 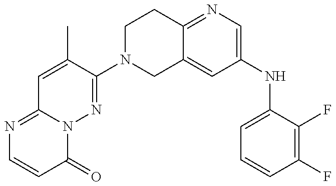 [SMILES: Cc1cc2nccc(=O)n2nc1N1CCc2ncc(Nc3cccc(F)c3F)cc2C1] | 421 |
| 2-107 | 7-(3-((6-fluoropyridin-2-yl)amino)- 7,8-dihydro-1,6-naphthyridin- 6(5H)-yl)-8-methyl-4H- pyrimido[1,2-b]pyridazin-4-one | 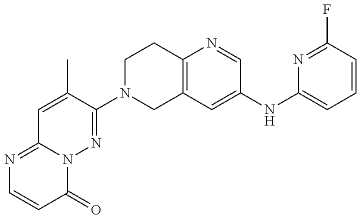 [SMILES: Cc1cc2nccc(=O)n2nc1N1CCc2ncc(Nc3cccc(F)n3)cc2C1] | 431 |
| 2-108 | N-(6-(8-methyl-4-oxo-4H- pyrimido[1,2-b]pyridazin-7-yl)- 5,6,7,8-tetrahydro-1,6- naphthyridin-3- yl)cyclopropanecarboxamide | 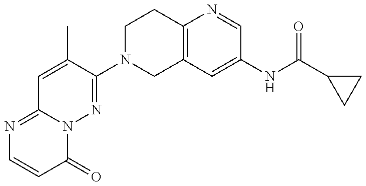 [SMILES: Cc1cc2nccc(=O)n2nc1N1CCc2ncc(NC(=O)C3CC3)cc2C1] | 377 |
| 2-109 | N-(6-(8-methyl-4-oxo-4H- pyrimido[1,2-b]pyridazin-7-yl)- 5,6,7,8-tetrahydro-1,6- naphthyridin-3-yl)oxetane-3- carboxamide | 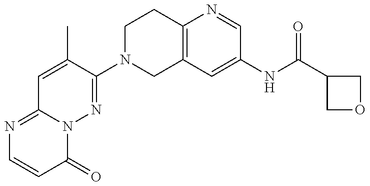 [SMILES: Cc1cc2nccc(=O)n2nc1N1CCc2ncc(NC(=O)C3COC3)cc2C1] | 393 |
| 2-110 | N-(6-(8-methyl-4-oxo-4H- pyrimido[1,2-b]pyridazin-7-yl)- 5,6,7,8-tetrahydro-1,6- naphthyridin-3-yl)tetrahydro-2H- pyran-4-carboxamide | 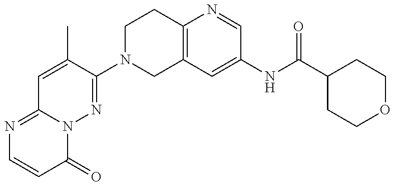 [SMILES: Cc1cc2nccc(=O)n2nc1N1CCc2ncc(NC(=O)C3CCOCC3)cc2C1] | 421 |
| 2-111 | 7-(3-((3-fluoropyridin-4-yl)amino)- 7,8-dihydro-1,6-naphthyridin- 6(5H)-yl)-8-methyl-4H- pyrimido[1,2-b]pyridazin-4-one | 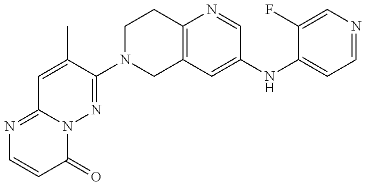 [SMILES: Cc1cc2nccc(=O)n2nc1N1CCc2ncc(Nc3ccncc3F)cc2C1] | 404 |
| 2-112 | 7-(3-((3-methoxypyridin-4- yl)amino)-7,8-dihydro-1,6- naphthyridin-6(5H)-yl)-8-methyl- 4H-pyrimido[1,2-b]pyridazin-4-one | 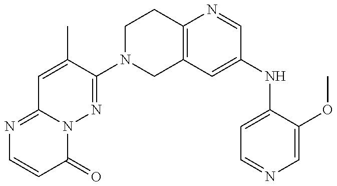 [SMILES: COc1cnccc1Nc1cnc2c(c1)CN(c1nn3c(=O)ccnc3cc1C)CC2] | 416 |
| 2-113 | 7-(3-((3-chloropyridin-4-yl)amino)- 7,8-dihydro-1,6-naphthyridin- 6(5H)-yl)-8-methyl-4H- pyrimido[1,2-b]pyridazin-4-one | 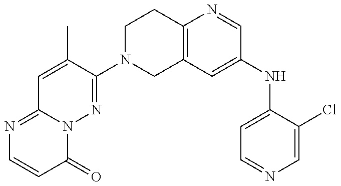 [SMILES: Cc1cc2nccc(=O)n2nc1N1CCc2ncc(Nc3ccncc3Cl)cc2C1] | 420 |
| 2-114 | 7-(3-((4-fluoro-2-methylpyridin-3- yl)amino)-7,8-dihydro-1,6- naphthyridin-6(5H)-yl)-8-methyl- 4H-pyrimido[1,2-b]pyridazin-4-one | 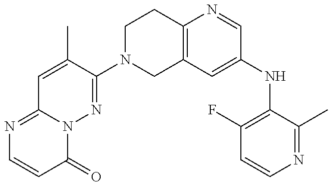 [SMILES: Cc1cc2nccc(=O)n2nc1N1CCc2ncc(Nc3c(F)ccnc3C)cc2C1] | 418 |
| 2-115 | 7-(3-((2-fluoro-6-methylpyridin-3- yl)amino)-7,8-dihydro-1,6- naphthyridin-6(5H)-yl)-8-methyl- 4H-pyrimido[1,2-b]pyridazin-4-one | 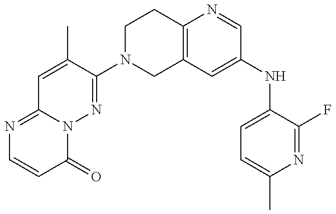 [SMILES: Cc1ccc(Nc2cnc3c(c2)CN(c2nn4c(=O)ccnc4cc2C)CC3)c(F)n1] | 418 |
| 2-116 | 7-(3-((5-chloro-2-fluoropyridin-3- yl)amino)-7,8-dihydro-1,6- naphthyridin-6(5H)-yl)-8-methyl- 4H-pyrimido[1,2-b]pyridazin-4-one | 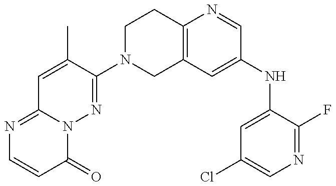 [SMILES: Cc1cc2nccc(=O)n2nc1N1CCc2ncc(Nc3cc(Cl)cnc3F)cc2C1] | 438 |
| 2-117 | N-(6-(8-methyl-4-oxo-4H- pyrimido[1,2-b]pyridazin-7-yl)- 5,6,7,8-tetrahydro-1,6- naphthyridin-3- yl)methanesulfonamide | 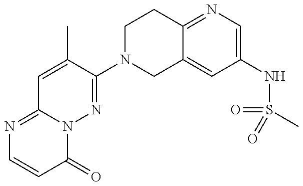 [SMILES: Cc1cc2nccc(=O)n2nc1N1CCc2ncc(NS(C)(=O)=O)cc2C1] | 387 |
| 2-118 | N-(6-(8-methyl-4-oxo-4H- pyrimido[1,2-b]pyridazin-7-yl)- 5,6,7,8-tetrahydro-1,6- naphthyridin-3- yl)cyclopropanesulfonamide | 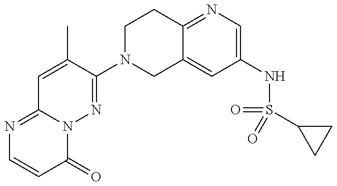 [SMILES: Cc1cc2nccc(=O)n2nc1N1CCc2ncc(NS(=O)(=O)C3CC3)cc2C1] | 413 |
| 2-119 | 2-methyl-N-(6-(8-methyl-4-oxo- 4H-pyrimido[1,2-b]pyridazin-7-yl)- 5,6,7,8-tetrahydro-1,6- naphthyridin-3-yl)thiazole-4- sulfonamide | 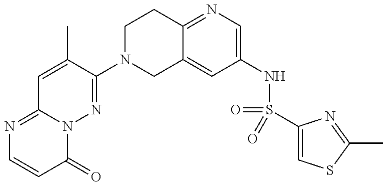 [SMILES: Cc1nc(S(=O)(=O)Nc2cnc3c(c2)CN(c2nn4c(=O)ccnc4cc2C)CC3)cs1] | 470 |
| 2-120 | 3,3-difluoro-N-(6-(8-methyl-4-oxo- 4H-pyrimido[1,2-b]pyridazin-7-yl)- 5,6,7,8-tetrahydro-1,6- naphthyridin-3-yl)cyclobutane-1- carboxamide | 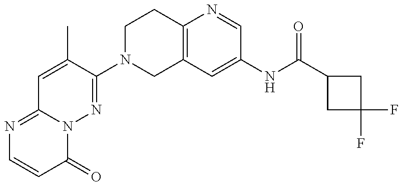 [SMILES: Cc1cc2nccc(=O)n2nc1N1CCc2ncc(NC(=O)C3CC(F)(F)C3)cc2C1] | 427 |
| 2-121 | 1-methyl-N-(6-(8-methyl-4-oxo- 4H-pyrimido[1,2-b]pyridazin-7-yl)- 5,6,7,8-tetrahydro-1,6- naphthyridin-3-yl)cyclobutane-1- carboxamide | 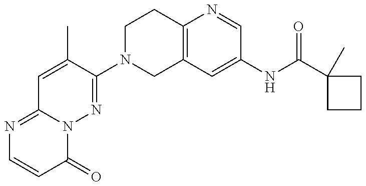 [SMILES: Cc1cc2nccc(=O)n2nc1N1CCc2ncc(NC(=O)C3(C)CCC3)cc2C1] | 405 |
| 2-122 | N-(6-(8-methyl-4-oxo-4H- pyrimido[1,2-b]pyridazin-7-yl)- 5,6,7,8-tetrahydro-1,6- naphthyridin-3-yl)oxazole-2- carboxamide | 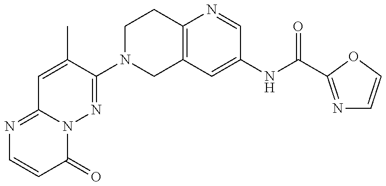 [SMILES: Cc1cc2nccc(=O)n2nc1N1CCc2ncc(NC(=O)c3ncco3)cc2C1] | 404 |
| 2-123 | 7-(3-((2-fluoropyridin-4-yl)amino)- 7,8-dihydro-1,6-naphthyridin- 6(5H)-yl)-8-methyl-4H- pyrimido[1,2-b]pyridazin-4-one | 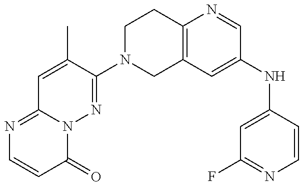 [SMILES: Cc1cc2nccc(=O)n2nc1N1CCc2ncc(Nc3ccnc(F)c3)cc2C1] | 404 |
| 2-124 | 8-methyl-7-(3-((4-methylpyridin-3- yl)amino)-7,8-dihydro-1,6- naphthyridin-6(5H)-yl)-4H- pyrimido[1,2-b]pyridazin-4-one | 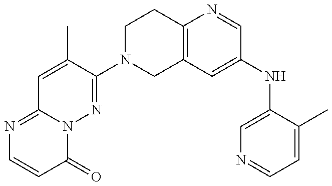 [SMILES: Cc1ccncc1Nc1cnc2c(c1)CN(c1nn3c(=O)ccnc3cc1C)CC2] | 400 |
| 2-125 | 7-(3-((6-cyclopropylpyridin-3- yl)amino)-7,8-dihydro-1,6- naphthyridin-6(5H)-yl)-8-methyl- 4H-pyrimido[1,2-b]pyridazin-4-one | 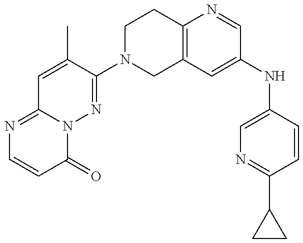 [SMILES: Cc1cc2nccc(=O)n2nc1N1CCc2ncc(Nc3ccc(C4CC4)nc3)cc2C1] | 426 |
| 2-126 | 7-(3-((6-chloropyridin-3-yl)amino)- 7,8-dihydro-1,6-naphthyridin- 6(5H)-yl)-8-methyl-4H- pyrimido[1,2-b]pyridazin-4-one | 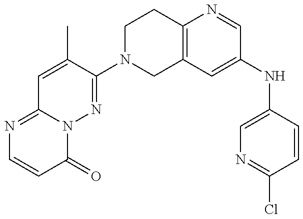 [SMILES: Cc1cc2nccc(=O)n2nc1N1CCc2ncc(Nc3ccc(Cl)nc3)cc2C1] | 420 |
| 2-127 | 8-methyl-7-(3-(pyrazin-2-ylamino)- 7,8-dihydro-1,6-naphthyridin- 6(5H)-yl)-4H-pyrimido[1,2- b]pyridazin-4-one | 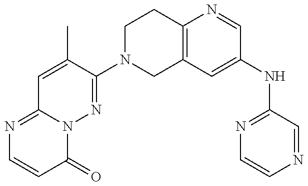 [SMILES: Cc1cc2nccc(=O)n2nc1N1CCc2ncc(Nc3cnccn3)cc2C1] | 387 |
| 2-128 | 7-(3-((5-fluoropyridin-3-yl)amino)- 7,8-dihydro-1,6-naphthyridin- 6(5H)-yl)-8-methyl-4H- pyrimido[1,2-b]pyridazin-4-one | 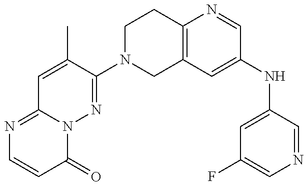 [SMILES: Cc1cc2nccc(=O)n2nc1N1CCc2ncc(Nc3cncc(F)c3)cc2C1] | 404 |
| 2-129 | 8-methyl-7-(3-(((2-methyloxazol-5- yl)methyl)amino)-7,8-dihydro-1,6- naphthyridin-6(5H)-yl)-4H- pyrimido[1,2-b]pyridazin-4-one | 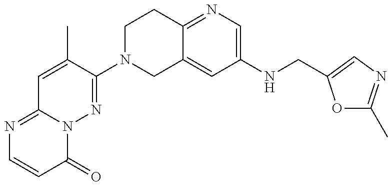 [SMILES: Cc1ncc(CNc2cnc3c(c2)CN(c2nn4c(=O)ccnc4cc2C)CC3)o1] | 404 |
| 2-130 | 8-methyl-7-(3-((thiazol-4- ylmethyl)amino)-7,8-dihydro-1,6- naphthyridin-6(5H)-yl)-4H- pyrimido[1,2-b]pyridazin-4-one | 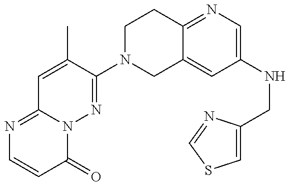 [SMILES: Cc1cc2nccc(=O)n2nc1N1CCc2ncc(NCc3cscn3)cc2C1] | 406 |
| 2-131 | 8-methyl-7-(3-(((1-methyl-1H- imidazol-5-yl)methyl)amino)-7,8- dihydro-1,6-naphthyridin-6(5H)- yl)-4H-pyrimido[1,2-b]pyridazin-4- one | 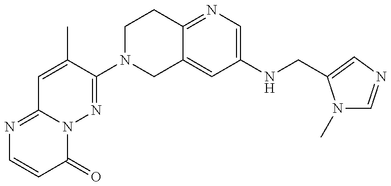 [SMILES: Cc1cc2nccc(=O)n2nc1N1CCc2ncc(NCc3cncn3C)cc2C1] | 403 |
| 2-132 | 7-(3-((6-chloro-5-methylpyridin-3- yl)amino)-7,8-dihydro-1,6- naphthyridin-6(5H)-yl)-8-methyl- 4H-pyrimido[1,2-b]pyridazin-4-one | 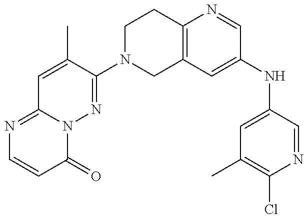 [SMILES: Cc1cc(Nc2cnc3c(c2)CN(c2nn4c(=O)ccnc4cc2C)CC3)cnc1Cl] | 435 |
| 2-133 | 8-methyl-7-(3-((5-methylpyridin-3- yl)amino)-7,8-dihydro-1,6- naphthyridin-6(5H)-yl)-4H- pyrimido[1,2-b]pyridazin-4-one | 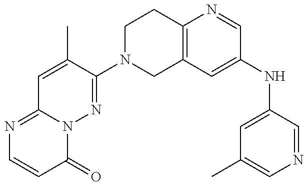 [SMILES: Cc1cncc(Nc2cnc3c(c2)CN(c2nn4c(=O)ccnc4cc2C)CC3)c1] | 400 |
| 2-134 | 7-(3-((6-methoxypyridin-3- yl)amino)-7,8-dihydro-1,6- naphthyridin-6(5H)-yl)-8-methyl- 4H-pyrimido[1,2-b]pyridazin-4-one | 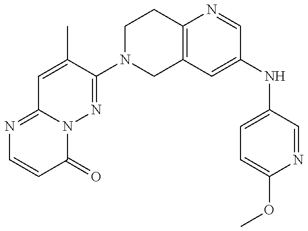 [SMILES: COc1ccc(Nc2cnc3c(c2)CN(c2nn4c(=O)ccnc4cc2C)CC3)cn1] | 416 |
| 2-135 | 7-(3-((4-chloropyridin-3-yl)amino)- 7,8-dihydro-1,6-naphthyridin- 6(5H)-yl)-8-methyl-4H- pyrimido[1,2-b]pyridazin-4-one | 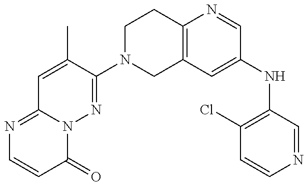 [SMILES: Cc1cc2nccc(=O)n2nc1N1CCc2ncc(Nc3cnccc3Cl)cc2C1] | 420 |
| 2-136 | 8-methyl-7-(3-(pyridazin-4- ylamino)-7,8-dihydro-1,6- naphthyridin-6(5H)-yl)-4H- pyrimido[1,2-b]pyridazin-4-one | 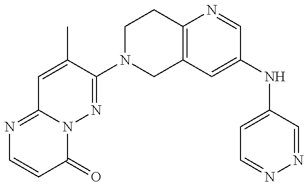 [SMILES: Cc1cc2nccc(=O)n2nc1N1CCc2ncc(Nc3ccnnc3)cc2C1] | 387 |
| 2-137 | 7-(3-((5-methoxypyridin-3- yl)amino)-7,8-dihydro-1,6- naphthyridin-6(5H)-yl)-8-methyl- 4H-pyrimido[1,2-b]pyridazin-4-one | 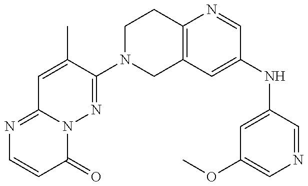 [SMILES: COc1cncc(Nc2cnc3c(c2)CN(c2nn4c(=O)ccnc4cc2C)CC3)c1] | 416 |
| 2-138 | 8-methyl-7-(3-((5- (trifluoromethyl)pyridin-3- yl)amino)-7,8-dihydro-1,6- naphthyridin-6(5H)-yl)-4H- pyrimido[1,2-b]pyridazin-4-one | 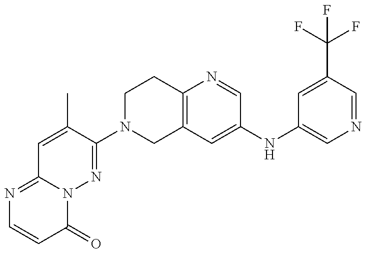 [SMILES: Cc1cc2nccc(=O)n2nc1N1CCc2ncc(Nc3cncc(C(F)(F)F)c3)cc2C1] | 454 |
| 2-139 | 8-methyl-7-(3-((4- methylpyrimidin-5-yl)amino)-7,8- dihydro-1,6-naphthyridin-6(5H)- yl)-4H-pyrimido[1,2-b]pyridazin-4- one | 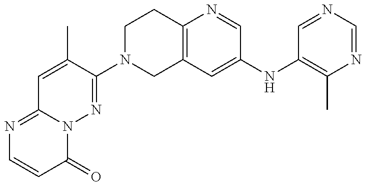 [SMILES: Cc1cc2nccc(=O)n2nc1N1CCc2ncc(Nc3cncnc3C)cc2C1] | 401 |
| 2-140 | 7-(3-((4-methoxypyrimidin-2- yl)amino)-7,8-dihydro-1,6- naphthyridin-6(5H)-yl)-8-methyl- 4H-pyrimido[1,2-b]pyridazin-4-one | 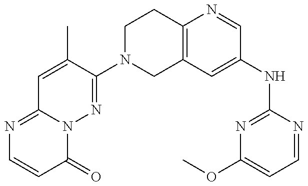 [SMILES: COc1ccnc(Nc2cnc3c(c2)CN(c2nn4c(=O)ccnc4cc2C)CC3)n1] | 417 |
| 2-141 | 7-(3-((2-methoxypyridin-4- yl)amino)-7,8-dihydro-1,6- naphthyridin-6(5H)-yl)-8-methyl- 4H-pyrimido[1,2-b]pyridazin-4-one | 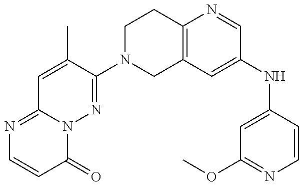 [SMILES: COc1cc(Nc2cnc3c(c2)CN(c2nn4c(=O)ccnc4cc2C)CC3)ccn1] | 416 |
| 2-142 | 7-(3-((5-chloropyridin-3-yl)amino)- 7,8-dihydro-1,6-naphthyridin- 6(5H)-yl)-8-methyl-4H- pyrimido[1,2-b]pyridazin-4-one | 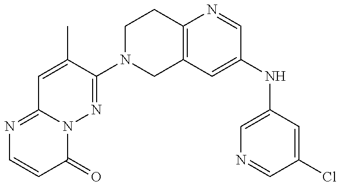 [SMILES: Cc1cc2nccc(=O)n2nc1N1CCc2ncc(Nc3cncc(Cl)c3)cc2C1] | 420 |
| 2-143 | 7-(3-((6-fluoro-2-methylpyridin-3- yl)amino)-7,8-dihydro-1,6- naphthyridin-6(5H)-yl)-8-methyl- 4H-pyrimido[1,2-b]pyridazin-4-one | 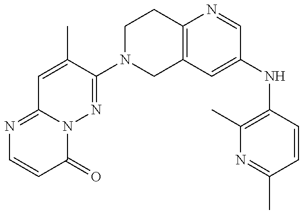 [SMILES: Cc1ccc(Nc2cnc3c(c2)CN(c2nn4c(=O)ccnc4cc2C)CC3)c(C)n1] | 418 |
| 2-144 | 3-(6-(8,9-dimethyl-4-oxo-4H- pyrimido[1,2-b]pyridazin-7-yl)- 5,6,7,8-tetrahydro-1,6- naphthyridin-3-yl)oxazolidin-2-one | 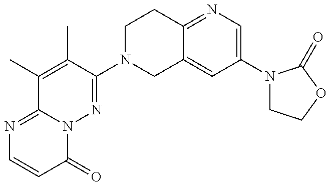 [SMILES: Cc1c(N2CCc3ncc(N4CCOC4=O)cc3C2)nn2c(=O)ccnc2c1C] | 393.1 |
| 2-145 | 7-(3-(4,4-dimethyl-2-oxopyrrolidin- 1-yl)-7,8-dihydro-1,6-naphthyridin- 6(5H)-yl)-8,9-dimethyl-4H- pyrimido[1,2-b]pyridazin-4-one | 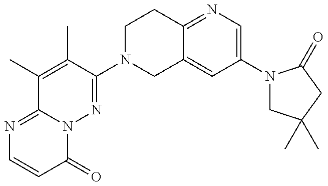 [SMILES: Cc1c(N2CCc3ncc(N4CC(C)(C)CC4=O)cc3C2)nn2c(=O)ccnc2c1C] | 419.2 |
| 2-146 | 7-(3-(1,1-dioxidoisothiazolidin-2- yl)-7,8-dihydro-1,6-naphthyridin- 6(5H)-yl)-8,9-dimethyl-4H- pyrimido[1,2-b]pyridazin-4-one | 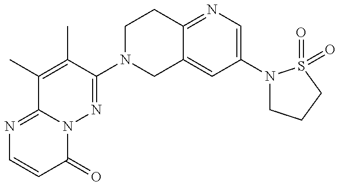 [SMILES: Cc1c(N2CCc3ncc(N4CCCS4(=O)=O)cc3C2)nn2c(=O)ccnc2c1C] | 427.1 |
| 2-147 | 8,9-dimethyl-7-(3-((1- oxidotetrahydro-126-thiophen-1- ylidene)amino)-7,8-dihydro-1,6- naphthyridin-6(5H)-yl)-4H- pyrimido[1,2-b]pyridazin-4-one | 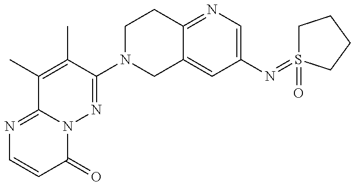 [SMILES: Cc1c(N2CCc3ncc(N=S4(=O)CCCC4)cc3C2)nn2c(=O)ccnc2c1C] | 425.2 |
| 2-148 | 7-(3-(3,3-dimethyl-2-oxopyrrolidin- 1-yl)-7,8-dihydro-1,6-naphthyridin- 6(5H)-yl)-8,9-dimethyl-4H- pyrimido[1,2-b]pyridazin-4-one | 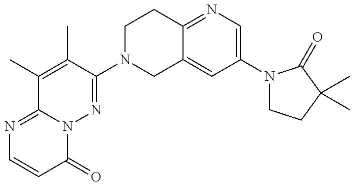 [SMILES: Cc1c(N2CCc3ncc(N4CCC(C)(C)C4=O)cc3C2)nn2c(=O)ccnc2c1C] | 419.2 |
| 2-149 | 8,9-dimethyl-7-(3-(2-oxo-2,3- dihydro-1H-pyrrolo[3,2-b]pyridin- 1-yl)-7,8-dihydro-1,6-naphthyridin- 6(5H)-yl)-4H-pyrimido[1,2- b]pyridazin-4-one | 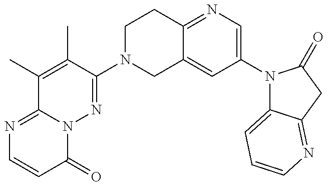 [SMILES: Cc1c(N2CCc3ncc(N4C(=O)Cc5ncccc54)cc3C2)nn2c(=O)ccnc2c1C] | 440.2 |
| 2-150 | 3-(6-(8,9-dimethyl-4-oxo-4H- pyrimido[1,2-b]pyridazin-7-yl)- 5,6,7,8-tetrahydro-1,6- naphthyridin-3-yl)-5,5- dimethyloxazolidin-2-one | 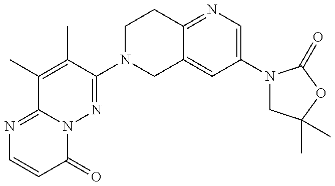 [SMILES: Cc1c(N2CCc3ncc(N4CC(C)(C)OC4=O)cc3C2)nn2c(=O)ccnc2c1C] | 421.2 |
| 2-151 | 7-(3-(2-oxa-6-azaspiro[3.3]heptan- 6-yl)-7,8-dihydro-1,6-naphthyridin- 6(5H)-yl)-8-methyl-4H- pyrimido[1,2-b]pyridazin-4-one | 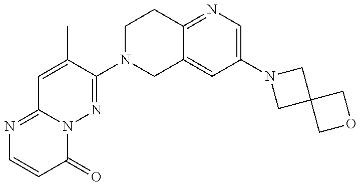 [SMILES: Cc1cc2nccc(=O)n2nc1N1CCc2ncc(N3CC4(COC4)C3)cc2C1] | 391 |
| 2-152 | 7-(3-((5-fluoro-2- (trifluoromethyl)pyridin-4- yl)amino)-7,8-dihydro-1,6- naphthyridin-6(5H)-yl)-8-methyl- 4H-pyrimido[1,2-b ]pyridazin-4-one | 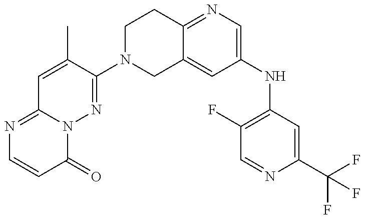 [SMILES: Cc1cc2nccc(=O)n2nc1N1CCc2ncc(Nc3cc(C(F)(F)F)ncc3F)cc2C1] | 472 |
| 2-153 | 7-(3-((3-fluoro-2-methylpyridin-4- yl)amino)-7,8-dihydro-1,6- naphthyridin-6(5H)-yl)-8-methyl- 4H-pyrimido[1,2-b ]pyridazin-4-one | 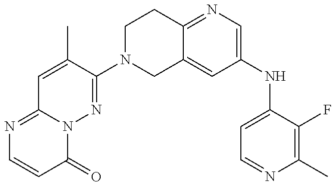 [SMILES: Cc1cc2nccc(=O)n2nc1N1CCc2ncc(Nc3ccnc(C)c3F)cc2C1] | 418 |
| 2-154 | 7-(3-((3,5-difluoropyridin-4- yl)amino)-7,8-dihydro-1,6- naphthyridin-6(5H)-yl)-8-methyl- 4H-pyrimido[1,2-b ]pyridazin-4-one | 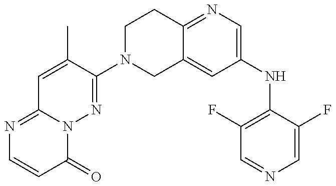 [SMILES: Cc1cc2nccc(=O)n2nc1N1CCc2ncc(Nc3c(F)cncc3F)cc2C1] | 422 |
| 2-155 | 7-(3-((5-fluoro-2-methylpyridin-4- yl)amino)-7,8-dihydro-1,6- naphthyridin-6(5H)-yl)-8-methyl- 4H-pyrimido[1,2-b ]pyridazin-4-one | 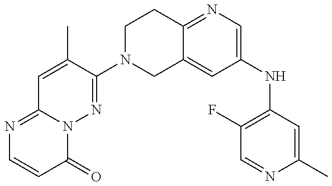 [SMILES: Cc1cc(Nc2cnc3c(c2)CN(c2nn4c(=O)ccnc4cc2C)CC3)c(F)cn1] | 418 |
| 2-156 | 3-fluoro-7-(3-((3-fluoropyridin-4- yl)amino)-7,8-dihydro-1,6- naphthyridin-6(5H)-yl)-2,8- dimethyl-4H-pyrimido[1,2- b]pyridazin-4-one | 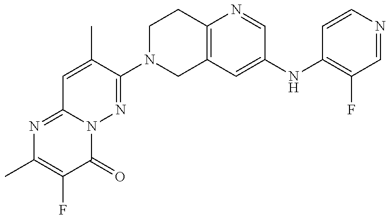 [SMILES: Cc1cc2nc(C)c(F)c(=O)n2nc1N1CCc2ncc(Nc3ccncc3F)cc2C1] | 436.3 |
| 2-157 | 3-chloro-7-(3-((3-fluoropyridin-4- yl)amino)-7,8-dihydro-1,6- naphthyridin-6(5H)-yl)-8-methyl- 4H-pyrimido[1,2-b ]pyridazin-4-one | 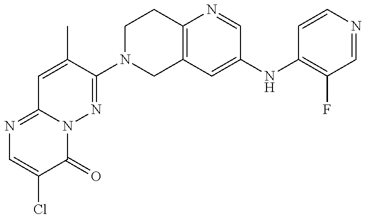 [SMILES: Cc1cc2ncc(Cl)c(=O)n2nc1N1CCc2ncc(Nc3ccncc3F)cc2C1] | 438.3 |
| 2-158 | 7-(3-((3-fluoropyridin-4-yl)amino)- 7,8-dihydro-1,6-naphthyridin- 6(5H)-yl)-2,3,8-trimethyl-4H- pyrimido[1,2-b]pyridazin-4-one | 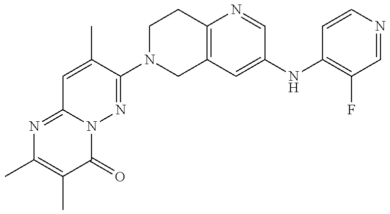 [SMILES: Cc1cc2nc(C)c(C)c(=O)n2nc1N1CCc2ncc(Nc3ccncc3F)cc2C1] | 432.5 |
| 2-159 | 2-(difluoromethyl)-7-(3-((3- fluoropyridin-4-yl)amino)-7,8- dihydro-1,6-naphthyridin-6(5H)- yl)-8-methyl-4H-pyrimido[1,2- b]pyridazin-4-one | 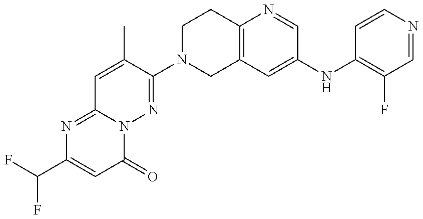 [SMILES: Cc1cc2nc(C(F)F)cc(=O)n2nc1N1CCc2ncc(Nc3ccncc3F)cc2C1] | 454.3 |
| 2-160 | 7-(3-((3-fluoropyridin-4-yl)amino)- 7,8-dihydro-1,6-naphthyridin- 6(5H)-yl)-8-methyl-2- (trifluoromethyl)-4H-pyrimido[1,2- b]pyridazin-4-one | 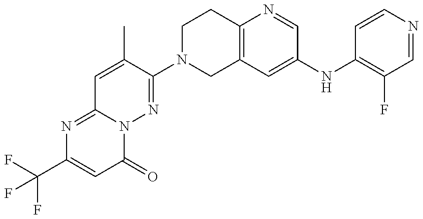 [SMILES: Cc1cc2nc(C(F)(F)F)cc(=O)n2nc1N1CCc2ncc(Nc3ccncc3F)cc2C1] | 472.4 |
| 2-161 | 3-chloro-7-(3-((3-fluoropyridin-4- yl)amino)-7,8-dihydro-1,6- naphthyridin-6(5H)-yl)-2,8- dimethyl-4H-pyrimido[1,2- b]pyridazin-4-one | 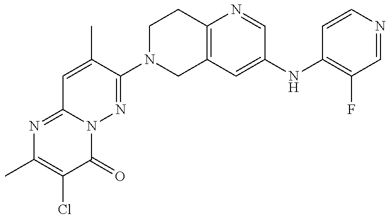 [SMILES: Cc1cc2nc(C)c(Cl)c(=O)n2nc1N1CCc2ncc(Nc3ccncc3F)cc2C1] | 452.3 |
| 2-162 | 7-(3-((5-fluoro-2-methylpyridin-4- yl)amino)-7,8-dihydro-1,6- naphthyridin-6(5H)-yl-5,5,7,7-d₄)- 8-methyl-4H-pyrimido[1,2- b]pyridazin-4-one | 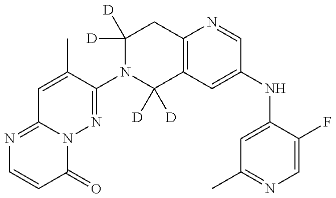 [SMILES: [2H]C1([2H])Cc2ncc(Nc3cc(C)ncc3F)cc2C([2H])([2H])N1c1nn2c(=O)ccnc2cc1C] | 422 |
| 2-163 | 8-methyl-7-(3-((2-methylpyridin-3- yl)amino)-7,8-dihydro-1,6- naphthyridin-6(5H)-yl-5,5,7,7-d₄)- 4H-pyrimido[1,2-b ]pyridazin-4-one | 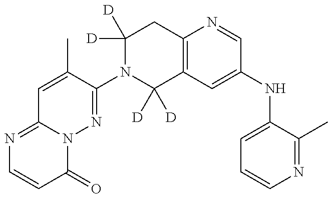 [SMILES: [2H]C1([2H])Cc2ncc(Nc3cccnc3C)cc2C([2H])([2H])N1c1nn2c(=O)ccnc2cc1C] | 404 |
| 2-164 | 7-(3-((3-fluoropyridin-4-yl)amino)- 7,8-dihydro-1,6-naphthyridin- 6(5H)-yl-5,5,7,7-d₄)-8-methyl-4H- pyrimido[1,2-b]pyridazin-4-one | 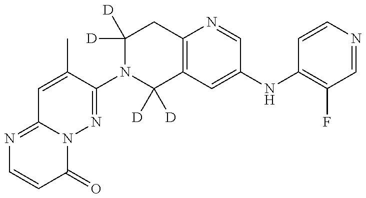 [SMILES: [2H]C1([2H])Cc2ncc(Nc3ccncc3F)cc2C([2H])([2H])N1c1nn2c(=O)ccnc2cc1C] | 408 |
| 2-165 | 8-methyl-7-(3-((3-methylpyridin-4- yl)amino)-7,8-dihydro-1,6- naphthyridin-6(5H)-yl-5,5,7,7-d₄)- 4H-pyrimido[1,2-b ]pyridazin-4-one | 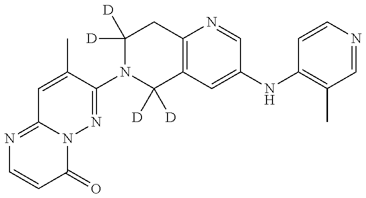 [SMILES: [2H]C1([2H])Cc2ncc(Nc3ccncc3C)cc2C([2H])([2H])N1c1nn2c(=O)ccnc2cc1C] | 404 |
| 2-166 | 7-(3-((2-fluorophenyl)amino)-7,8- dihydro-1,6-naphthyridin-6(5H)-yl- 5,5,7,7-d₄)-8-methyl-4H- pyrimido[1,2-b]pyridazin-4-one | 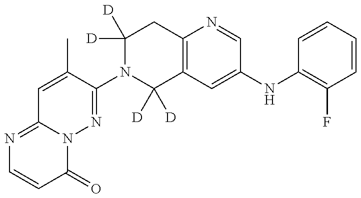 [SMILES: [2H]C1([2H])Cc2ncc(Nc3ccccc3F)cc2C([2H])([2H])N1c1nn2c(=O)ccnc2cc1C] | 407 |

d. Exemplified Compounds of the Invention

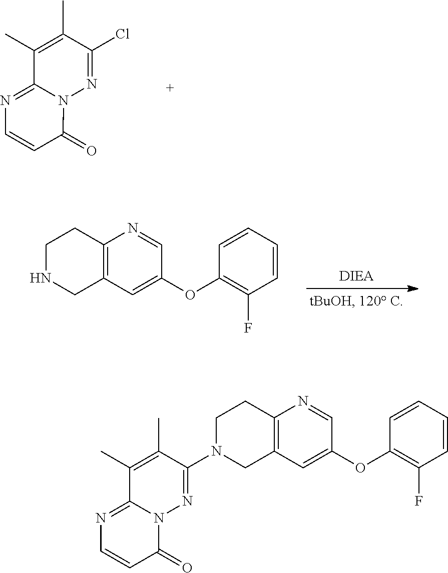 [SMILES: Cc1c(Cl)nn2c(=O)ccnc2c1C ; Fc1ccccc1Oc1cnc2c(c1)CNCC2 ; Cc1c(N2CCc3ncc(Oc4ccccc4F)cc3C2)nn2c(=O)ccnc2c1C]

7-(3-(2-Fluorophenoxy)-7,8-dihydro-1,6-naphthyridin-6(5H)-yl)-8,9-dimethyl-4H-pyrimido[1,2-b]pyridazin-4-one (Compound 3-3). To a solution of 7-chloro-8,9-dimethyl-4H-pyrimido[1,2-b]pyridazin-4-one (10 mg, 0.048 mmol) in tert-butanol (1.5 mL) was added DIEA (0.042 mL, 0.24 mmol) and 3-(2-fluorophenoxy)-5,6,7,8-tetrahydro-1,6-naphthyridine (23 mg, 0.095 mmol). The mixture was heated to 120° C. for 18 h. Solvents were removed, the crude product was dissolved in DMSO (2 mL), and purified by reverse-phase chromatography (MeCN/0.100 aqueous TFA) to afford the title compound (5.7 mg). ¹H NMR (400 MHz, CDCl₃) δ 8.31 (d, J=2.8 Hz, 1H), 8.16 (d, J=6.3 Hz, 1H), 7.24-7.08 (m, 4H), 7.05 (d, J=2.9 Hz, 1H), 6.56 (d, J=6.3 Hz, 1H), 4.59 (s, 2H), 3.50 (t, J=5.9 Hz, 2H), 3.23 (t, J=5.9 Hz, 2H), 2.56 (d, J=1.1 Hz, 3H), 2.43 (d, J=1.0 Hz, 3H); ES-MS [M+H]⁺=418.3.

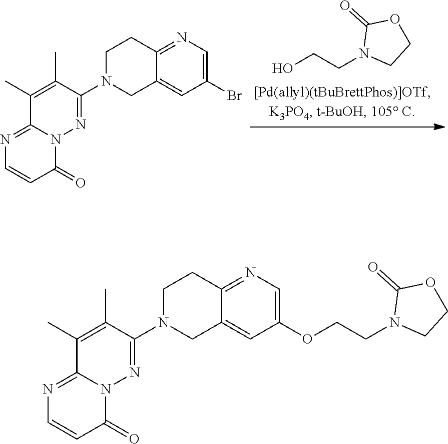 [SMILES: Cc1c(N2CCc3ncc(Br)cc3C2)nn2c(=O)ccnc2c1C ; O=C1OCCN1CCO ; Cc1c(N2CCc3ncc(OCCN4CCOC4=O)cc3C2)nn2c(=O)ccnc2c1C]

3-(2-((6-(8,9-Dimethyl-4-oxo-4H-pyrimido[1,2-b]pyridazin-7-yl)-5,6,7,8-tetrahydro-1,6-naphthyridin-3-yl)oxy)ethyl)oxazolidin-2-one (Compound 3-4). 7-(3-Bromo-7,8-dihydro-1,6-naphthyridin-6(5H)-yl)-8,9-dimethyl-4H-pyrimido[1,2-b]pyridazin-4-one (20 mg, 0.052 mmol), 3-(2-hydroxyethyl)oxazolidin-2-one (20 mg, 0.16 mmol), potassium phosphate tribasic (19 mg, 0.088 mmol) and [Pd(allyl)(tBuBrettPhos)]OTf (4.0 mg, 0.005 mmol) were combined in a vial, which was sealed and placed under an inert atmosphere. t-BuOH (0.75 mL) was then added via syringe, and the resulting reaction mixture was stirred at 105° C. for 2 h, after which time the reaction mixture was cooled to ambient temperature, and the solvents were concentrated. Solids were removed by syringe filtration, and crude residue was purified by RP-HPLC (5-45% MeCN/0.05% aqueous NH₄OH). The fractions containing product were extracted with DCM, and the combined organic extracts were filtered through a phase separator, and concentrated to afford 2.4 mg of the title compound. ¹H NMR (400 MHz, CDCl₃) δ 8.19-8.17 (m, 2H), 7.08 (d, J=2.8 Hz, 1H), 6.58 (d, J=6.3 Hz, 1H), 4.64 (s, 2H), 4.39-4.35 (m, 2H), 4.19 (t, J=5.0 Hz, 2H), 3.81-3.77 (m, 2H), 3.71 (t, J=5.0 Hz, 2H), 3.51 (t, J=5.9 Hz, 2H), 3.19 (t, J=5.9 Hz, 2H), 2.57 (d, J=1.0 Hz, 3H), 2.44 (d, J=1.0 Hz, 3H); ES-MS [M+1]⁺: 437.2.

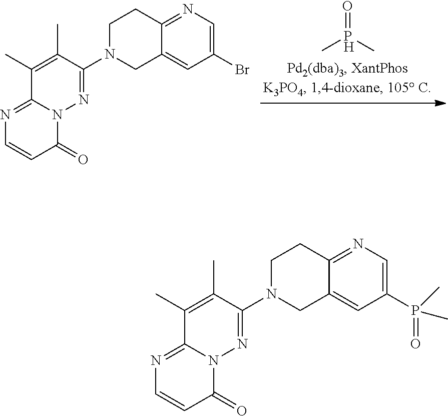

7-(3-(Dimethylphosphoryl)-7,8-dihydro-1,6-naphthyridin-6(5H)-yl)-8,9-dimethyl-4H-pyrimido[1,2-b]pyridazin-4-one (Compound 3-5). 7-(3-Bromo-7,8-dihydro-1,6-naphthyridin-6(5H)-yl)-8,9-dimethyl-4H-pyrimido[1,2-b]pyridazin-4-one (20 mg, 0.052 mmol), dimethylphosphine oxide (12 mg, 0.16 mmol), potassium phosphate tribasic (17 mg, 0.078 mmol), XantPhos (3.0 mg, 0.005 mmol), and Pd₂(dba)₃ (2.4 mg, 0.003 mmol) were combined in a vial, which was sealed and placed under an inert atmosphere. 1,4-Dioxane (1 mL) was then added via syringe, and the resulting reaction mixture was stirred at 105° C. for 3 h, after which time the reaction mixture was cooled to ambient temperature, and the solvents were concentrated. The solids were removed by syringe filtration, and the crude residue was purified by RP-HPLC (2-32% MeCN/0.1% aqueous TFA). The fractions containing product were basified with sat. NaHCO₃ solution and extracted with DCM. The combined organic extracts were filtered through a phase separator, and concentrated to afford 3.3 mg of the title compound. ¹H NMR (400 MHz, CDCl₃) δ 8.79 (dd, J=5.8, 2.0 Hz, 1H), 8.18 (d, J=6.3 Hz, 1H), 7.87 (dd, J=11.6, 2.0 Hz, 1H), 6.58 (d, J=6.3 Hz, 1H), 4.67 (s, 2H), 3.60 (t, J=5.9 Hz, 2H), 3.32 (t, J=5.9 Hz, 2H), 2.57 (d, J=1.0 Hz, 3H), 2.44 (d, J=1.0 Hz, 3H), 1.79 (d, J=13.1 Hz, 6H); ES-MS [M+1]⁺: 384.1.

The compounds shown in Table 3 may be prepared in an analogous manner with the appropriate starting materials.

TABLE 3

| Cpd. | | | ES-MS |
|---|---|---|---|
| No. | Name | Structure | [M + 1]⁺ |
| 3-1 | 7-(3-(2-fluorophenoxy)-7,8- dihydro-1,6-naphthyridin-6(5H)- yl)-8-methyl-4H-pyrimido[1,2- b]pyridazin-4-one | 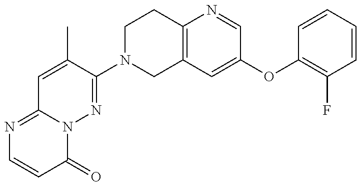 [SMILES: Cc1cc2nccc(=O)n2nc1N1CCc2ncc(Oc3ccccc3F)cc2C1] | 404.4 |
| 3-2 | 7-(3-(2-fluorophenoxy)-7,8- dihydro-1,6-naphthyridin-6(5H)- yl)-2,8-dimethyl-4H-pyrimido[1,2- b]pyridazin-4-one | 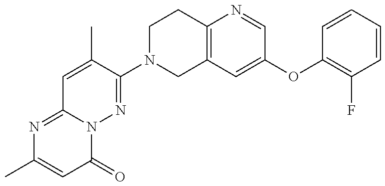 [SMILES: Cc1cc(=O)n2nc(N3CCc4ncc(Oc5ccccc5F)cc4C3)c(C)cc2n1] | 418.2 |
| 3-3 | 7-(3-(2-fluorophenoxy)-7,8- dihydro-1,6-naphthyridin-6(5H)- yl)-8,9-dimethyl-4H-pyrimido[1,2- b]pyridazin-4-one | 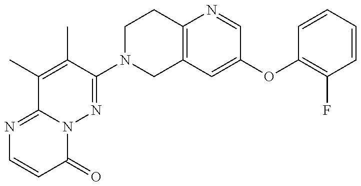 [SMILES: Cc1c(N2CCc3ncc(Oc4ccccc4F)cc3C2)nn2c(=O)ccnc2c1C] | 418.3 |
| 3-4 | 3-(2-((6-(8,9-dimethyl-4-oxo-4H- pyrimido[1,2-b]pyridazin-7-yl)- 5,6,7,8-tetrahydro-1,6- naphthyridin-3- yl)oxy)ethyl)oxazolidin-2-one | 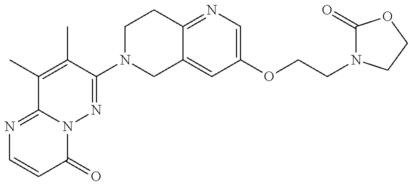 [SMILES: Cc1c(N2CCc3ncc(OCCN4CCOC4=O)cc3C2)nn2c(=O)ccnc2c1C] | 437.2 |
| 3-5 | 7-(3-(dimethylphosphoryl)-7,8- dihydro-1,6-naphthyridin-6(5H)- yl)-8,9-dimethyl-4H-pyrimido[1,2- b]pyridazin-4-one | 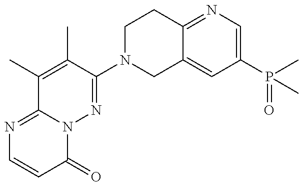 [SMILES: Cc1c(N2CCc3ncc(P(C)(C)=O)cc3C2)nn2c(=O)ccnc2c1C] | 384.1 |

### Biological Activity

### A. Cell Lines Expressing Muscarinic Acetylcholine Receptors

Human and rat M₄ cDNAs, along with the chimeric G protein G_{qi5}, were transfected into Chinese hamster ovary (CHO-K1) cells purchased from the American Type Culture Collection using Lipofectamine2000. hM₄-G_{qi5} cells were grown in Ham's F-12 medium containing 10% heat-inactivated fetal bovine serum (FBS), 20 mM HEPES, 50 μg/mL G418 sulfate, and 500 μg/mL Hygromycin B. rM₄-G_{qi5} cells were grown in DMEM containing 10% heat-inactivated FBS, 20 mM HEPES, 400 μg/mL G418 sulfate, and 500 μg/mL Hygromycin B.

### B. Cell-Based Functional Assay of Muscarinic Acetylcholine Receptor Activity

For high throughput measurement of agonist-evoked increases in intracellular calcium, CHO-K1 cells stably expressing muscarinic receptors were plated in growth medium lacking G418 and hygromycin at 15,000 cells/20 μL/well in Greiner 384-well black-walled, tissue culture (TC)-treated, clear-bottom plates (VWR). Cells were incubated overnight at 37° C. and 5% CO₂. The next day, cells were washed using an ELX 405 (BioTek) with assay buffer; the final volume was then aspirated to 20 μL. Next, 20 μL of a 2.3 M stock of Fluo-4/acetoxymethyl ester (Invitrogen, Carlsbad, CA), prepared as a 2.3 mM stock in DMSO and mixed in a 1:1 ratio with 10% (w/v) Pluronic F-127 and diluted in assay buffer, was added to the wells and the cell plates were incubated for 50 min at 37° C. and 5% CO₂. Dye was removed by washing with the ELX 405 and the final volume was aspirated to 20 μL. Compound master plates were formatted in an 11 point concentration-response curve (CRC) format (1:3 dilutions) in 100% DMSO with a starting concentration of 10 mM using a BRAVO liquid handler (Agilent). Test compound CRCs were then transferred to daughter plates (240 nL) using the Echo acoustic plate reformatter (Labcyte, Sunnyvale, CA) and then diluted into assay buffer (40 μL) to a 2× stock using a Thermo Fisher Combi (Thermo Fisher Scientific, Waltham, MA).

Calcium flux was measured using the Functional Drug Screening System (FDSS) 6000 or 7000 (Hamamatsu Corporation, Tokyo, Japan) as an increase in the fluorescent static ratio. Compounds were applied to cells (20 μL, 2X) using the automated system of the FDSS at 2-4 seconds into the protocol and the data were collected at 1 Hz. At 144 seconds, 10 μL of an EC₂₀ concentration of the muscarinic receptor agonist acetylcholine was added (5×), followed by the addition of 12 μL of an EC₂₀ concentration of acetylcholine at the 230 second time point (5×). Agonist activity was analyzed as a concentration-dependent increase in calcium mobilization upon compound addition. Positive allosteric modulator activity was analyzed as a concentration-dependent increase in the EC₂₀ acetylcholine response. Antagonist activity was analyzed as a concentration-dependent decrease in the EC₂₀ acetylcholine response. Concentration-response curves were generated using a four-parameter logistical equation in XLFit curve fitting software (IDBS, Bridgewater, NJ) for Excel (Microsoft, Redmond, WA) or Prism (GraphPad Software, Inc., San Diego, CA).

The above described assay was also operated in a second mode where an appropriate fixed concentration of the present compounds were added to the cells after establishment of a fluorescence baseline for about 3 seconds, and the response in cells was measured. 140 s later, the appropriate concentration of agonist was added and the calcium response (maximum-local minima response) was measured. The EC₅₀ values for the agonistin the presence of test compound were determined by nonlinear curve fitting. A decrease in the EC₅₀ value of the agonist with increasing concentrations of the present compounds (a leftward shift of the agonist concentration-response curve) is an indication of the degree of muscarinic positive allosteric modulation at a given concentration of the present compound. An increase in the EC₅₀ value of the agonist with increasing concentrations of the present compounds (a rightward shift of the agonist concentration response curve) is an indication of the degree of muscarinic antagonism at a given concentration of the present compound. The second mode also indicates whether the present compounds also affect the maximum response of the muscarinic receptor to agonists.

C. Activity of Compounds in a mAChR M₄ Cell-Based Assay

Compounds were synthesized as described above. Activity (EC₅₀ and Eₘₐₓ) was determined in the mAChR M₄ cell-based functional assay as described above and the data are shown in Table 4. The compound number corresponds to the compound numbers used in Tables 1-3.

TABLE 4

| | Human M₄ | Eₘₐₓ |
|---|---|---|
| No. | EC₅₀ (nM) | (%)* |
| 1-1 | 300 | 81 |
| 1-2 | 501 | 87 |
| 1-3 | 99 | 76 |
| 1-4 | 181 | 96 |
| 1-5 | 422 | 86 |
| 1-6 | 736 | 83 |
| 1-7 | 363 | 79 |
| 1-8 | 308 | 81 |
| 1-9 | 25 | 75 |
| 1-10 | 162 | 86 |
| 1-11 | 158 | 92 |
| 1-12 | 127 | 80 |
| 1-13 | 373 | 79 |
| 1-14 | 69 | 77 |
| 1-15 | 136 | 73 |
| 1-16 | 340 | 71 |
| 1-17 | 105 | 79 |
| 1-18 | 94 | 106 |
| 1-19 | 113 | 70 |
| 1-20 | 586 | 70 |
| 1-21 | 136 | 75 |
| 1-22 | 246 | 73 |
| 1-23 | 363 | 75 |
| 1-24 | 309 | 75 |
| 1-25 | 254 | 81 |
| 1-26 | 141 | 74 |
| 1-27 | 38 | 76 |
| 1-28 | 40 | 86 |
| 1-29 | 146 | 85 |
| 1-30 | 127 | 69 |
| 1-31 | 104 | 83 |
| 1-32 | 405 | 76 |
| 1-33 | 64 | 77 |
| 1-34 | 73 | 90 |
| 1-35 | 80 | 92 |
| 1-36 | 76 | 96 |
| 1-37 | 2531 | 83 |
| 1-38 | 338 | 84 |
| 1-39 | 206 | 86 |
| 1-40 | 38 | 93 |
| 1-41 | 430 | 83 |
| 1-42 | 200 | 87 |
| 1-43 | 612 | 92 |
| 1-44 | 1111 | 92 |
| 1-45 | 167 | 100 |
| 1-46 | 118 | 99 |
| 1-47 | 144 | 103 |
| 1-48 | 321 | 72 |
| 1-49 | 2339 | 98 |
| 1-50 | 685 | 84 |
| 1-51 | 293 | 85 |
| 1-52 | 232 | 47 |
| 1-53 | 699 | 75 |
| 1-54 | 479 | 76 |
| 1-55 | 481 | 94 |
| 1-56 | 281 | 102 |
| 1-57 | 735 | 70 |
| 1-58 | 397 | 88 |
| 1-59 | 244 | 99 |
| 1-60 | 140 | 104 |
| 1-61 | 71 | 96 |
| 1-62 | 128 | 89 |
| 1-63 | 203 | 97 |
| 1-64 | 91 | 102 |
| 1-65 | 656 | 70 |
| 1-66 | 832 | 77 |
| 1-67 | 328 | 80 |
| 1-68 | 358 | 84 |
| 1-69 | 769 | 87 |
| 1-70 | 274 | 92 |
| 1-71 | 145 | 90 |
| 1-72 | 174 | 75 |
| 1-73 | 457 | 77 |
| 1-74 | 445 | 102 |
| 1-75 | 1248 | 46 |
| 1-76 | 149 | 79 |
| 1-77 | 285 | 74 |
| 1-78 | 152 | 79 |
| 1-79 | 66 | 109 |
| 1-80 | 64 | 104 |
| 1-81 | 88 | 103 |
| 1-82 | 145 | 100 |
| 1-83 | 209 | 87 |
| 1-84 | 149 | 101 |
| 1-85 | 127 | 103 |
| 1-86 | 243 | 94 |
| 1-87 | 128 | 82 |
| 1-88 | 97 | 83 |
| 1-89 | 231 | 70 |
| 1-90 | 669 | 87 |
| 1-91 | 127 | 83 |
| 1-92 | 339 | 76 |
| 1-93 | 333 | 90 |
| 1-94 | 122 | 79 |
| 1-95 | 68 | 99 |
| 1-96 | 37 | 63 |
| 1-97 | 397 | 79 |
| 1-98 | 534 | 86 |
| 1-99 | 190 | 88 |
| 1-100 | 134 | 92 |
| 1-101 | 332 | 93 |
| 1-102 | 733 | 83 |
| 1-103 | 449 | 93 |
| 1-104 | 696 | 90 |
| 1-105 | 162 | 99 |
| 1-106 | 108 | 104 |
| 1-107 | 178 | 102 |
| 1-108 | 158 | 94 |
| 1-109 | 60 | 79 |
| 1-110 | 177 | 95 |
| 1-111 | 2453 | 61 |
| 1-112 | 668 | 67 |
| 1-113 | 262 | 73 |
| 1-114 | 615 | 73 |
| 1-115 | 313 | 91 |
| 1-116 | 19 | 93 |
| 1-117 | 198 | 90 |
| 1-118 | 6066 | 83 |
| 1-119 | 499 | 85 |
| 1-120 | 749 | 65 |
| 1-121 | 722 | 83 |
| 1-122 | 2930 | 93 |
| 1-123 | 858 | 89 |
| 1-124 | 3409 | 84 |
| 1-125 | 368 | 97 |
| 1-126 | 758 | 88 |
| 1-127 | 1240 | 86 |
| 1-128 | 768 | 80 |
| 1-129 | 1625 | 91 |
| 1-130 | 8095 | 84 |
| 1-131 | 1763 | 62 |
| 1-132 | 798 | 92 |
| 1-133 | 556 | 79 |
| 1-134 | 700 | 88 |
| 1-135 | 626 | 97 |
| 1-136 | 633 | 102 |
| 1-137 | 1726 | 109 |
| 1-138 | 190 | 87 |
| 1-139 | 77 | 98 |
| 1-140 | 360 | 89 |
| 1-141 | 410 | 96 |
| 1-142 | 371 | 53 |
| 1-143 | 416 | 102 |
| 1-144 | 246 | 82 |
| 1-145 | 122 | 88 |
| 1-146 | 399 | 86 |
| 1-147 | 305 | 80 |
| 1-148 | 344 | 89 |
| 1-149 | 68 | 89 |
| 1-150 | 511 | 74 |
| 1-151 | 3214 | 75 |
| 1-152 | 133 | 80 |
| 1-153 | 584 | 67 |
| 1-154 | 401 | 80 |
| 1-155 | 62 | 91 |
| 1-156 | 204 | 95 |
| 1-157 | 426 | 100 |
| 1-158 | 251 | 94 |
| 1-159 | 3065 | 86 |
| 1-160 | 547 | 82 |
| 1-161 | 92 | 76 |
| 1-162 | 520 | 71 |
| 1-163 | 1017 | 71 |
| 1-164 | 640 | 69 |
| 1-165 | 10000 | 47 |
| 1-166 | 283 | 86 |
| 1-167 | 353 | 80 |
| 1-168 | 2313 | 72 |
| 1-169 | 883 | 89 |
| 1-170 | 185 | 95 |
| 1-171 | 429 | 91 |
| 1-172 | 114 | 85 |
| 1-173 | 338 | 81 |
| 1-174 | 450 | 82 |
| 1-175 | 4659 | 61 |
| 1-176 | 4840 | 74 |
| 1-177 | 1417 | 84 |
| 1-178 | 2508 | 74 |
| 1-179 | 805 | 85 |
| 1-180 | 2473 | 63 |
| 1-181 | 648 | 95 |
| 1-182 | 1407 | 66 |
| 1-183 | 582 | 79 |
| 1-184 | 1261 | 80 |
| 1-185 | 752 | 86 |
| 1-186 | 460 | 84 |
| 1-187 | 109 | 95 |
| 1-188 | 103 | 91 |
| 1-189 | 515 | 48 |
| 1-190 | 156 | 77 |
| 1-191 | 2398 | 84 |
| 1-192 | 139 | 64 |
| 1-193 | 783 | 71 |
| 1-194 | 93 | 83 |
| 1-195 | 185 | 82 |
| 1-196 | 1131 | 80 |
| 1-197 | 275 | 81 |
| 1-198 | 1922 | 83 |
| 1-199 | 526 | 74 |
| 1-200 | 531 | 84 |
| 1-201 | 603 | 86 |
| 1-202 | 104 | 91 |
| 1-203 | 417 | 80 |
| 1-204 | 102 | 92 |
| 1-205 | 1244 | 81 |
| 1-206 | 409 | 71 |
| 1-207 | 315 | 91 |
| 1-208 | 518 | 86 |
| 1-209 | 266 | 88 |
| 1-210 | 287 | 84 |
| 1-211 | 446 | 76 |
| 1-212 | 867 | 83 |
| 1-213 | 443 | 82 |
| 1-214 | 139 | 82 |
| 1-215 | 338 | 85 |
| 1-216 | 380 | 79 |
| 1-217 | 655 | 60 |
| 1-218 | 793 | 92 |
| 1-219 | 341 | 80 |
| 1-220 | 747 | 70 |
| 1-221 | 170 | 58 |
| 1-222 | 74 | 83 |
| 1-223 | 618 | 64 |
| 1-224 | 651 | 60 |
| 1-225 | 232 | 66 |
| 1-226 | 1189 | 59 |
| 1-227 | 135 | 83 |
| 1-228 | 34 | 85 |
| 1-229 | 48 | 90 |
| 1-230 | 1305 | 43 |
| 1-231 | 859 | 56 |
| 1-232 | 441 | 57 |
| 1-233 | 735 | 65 |
| 1-234 | 235 | 71 |
| 1-235 | 568 | 62 |
| 1-236 | 1596 | 87 |
| 1-237 | 365 | 100 |
| 1-238 | 711 | 92 |
| 1-239 | 98 | 78 |
| 1-240 | 284 | 86 |
| 1-241 | 228 | 87 |
| 1-242 | 612 | 79 |
| 1-243 | 124 | 84 |
| 1-244 | 112 | 82 |
| 1-245 | 72 | 87 |
| 1-246 | 1020 | 80 |
| 1-247 | 1028 | 55 |
| 1-248 | 852 | 80 |
| 1-249 | 1003 | 87 |
| 1-250 | 1012 | 79 |
| 1-251 | 289 | 84 |
| 1-252 | 631 | 70 |
| 1-253 | 2329 | 54 |
| 1-254 | 1368 | 48 |
| 1-255 | 2367 | 60 |
| 1-256 | 578 | 71 |
| 1-257 | 339 | 76 |
| 1-258 | 363 | 67 |
| 1-259 | 445 | 72 |
| 1-260 | 97 | 83 |
| 1-261 | 20 | 94 |
| 1-262 | 1751 | 99 |
| 1-263 | 96 | 86 |
| 1-264 | 222 | 98 |
| 1-265 | 929 | 61 |
| 1-266 | 621 | 81 |
| 1-267 | 183 | 89 |
| 1-268 | 230 | 83 |
| 1-269 | 211 | 88 |
| 1-270 | 33 | 95 |
| 1-271 | 70 | 87 |
| 1-272 | 810 | 82 |
| 1-273 | 1088 | 75 |
| 1-274 | 448 | 84 |
| 1-275 | 1790 | 54 |
| 1-276 | 10000 | 66 |
| 1-277 | 1124 | 57 |
| 1-278 | 499 | 81 |
| 1-279 | 1123 | 81 |
| 1-280 | 296 | 52 |
| 1-281 | 498 | 85 |
| 1-282 | 481 | 90 |
| 1-283 | 900 | 79 |
| 1-284 | 1105 | 77 |
| 1-285 | 552 | 89 |
| 1-286 | 8724 | 68 |
| 1-287 | 1210 | 85 |
| 1-288 | 311 | 84 |
| 1-289 | 4548 | 70 |
| 1-290 | 375 | 90 |
| 1-291 | 356 | 82 |
| 1-292 | 477 | 92 |
| 1-293 | 331 | 88 |
| 1-294 | 245 | 99 |
| 1-295 | 89 | 96 |
| 1-296 | 563 | 62 |
| 1-297 | 76 | 87 |
| 1-298 | 83 | 85 |
| 1-299 | 41 | 93 |
| 1-300 | 122 | 87 |
| 1-301 | 111 | 91 |
| 1-302 | 188 | 85 |
| 1-303 | 192 | 96 |
| 1-304 | 169 | 89 |
| 1-305 | 315 | 88 |
| 1-306 | 451 | 89 |
| 1-307 | 69 | 88 |
| 1-308 | 313 | 78 |
| 1-309 | 203 | 88 |
| 1-310 | 136 | 86 |
| 1-311 | 1267 | 79 |
| 1-312 | 158 | 82 |
| 1-313 | 503 | 96 |
| 1-314 | 109 | 96 |
| 1-315 | 448 | 71 |
| 1-316 | 10000 | 71 |
| 1-317 | 716 | 70 |
| 1-318 | 2397 | 55 |
| 1-319 | 2639 | 34 |
| 1-320 | 1467 | 77 |
| 1-321 | 2178 | 61 |
| 1-322 | 374 | 82 |
| 1-323 | 1039 | 71 |
| 1-324 | 10000 | 59 |
| 1-325 | 921 | 68 |
| 1-326 | 1477 | 49 |
| 1-327 | 311 | 71 |
| 1-328 | 484 | 68 |
| 1-329 | 1058 | 78 |
| 1-330 | 477 | 68 |
| 1-331 | 1247 | 53 |
| 1-332 | 244 | 67 |
| 1-333 | 247 | 69 |
| 1-334 | 1232 | 45 |
| 1-335 | 576 | 68 |
| 1-336 | 357 | 66 |
| 1-337 | 331 | 76 |
| 1-338 | 131 | 88 |
| 1-339 | 230 | 77 |
| 1-340 | 503 | 48 |
| 1-341 | 673 | 75 |
| 1-342 | 204 | 72 |
| 1-343 | 400 | 77 |
| 1-344 | 663 | 72 |
| 1-345 | 200 | 70 |
| 1-346 | 483 | 67 |
| 1-347 | 217 | 80 |
| 1-348 | 2600 | 68 |
| 1-349 | 1144 | 69 |
| 1-350 | 3448 | 72 |
| 1-351 | 426 | 55 |
| 1-352 | 3311 | 59 |
| 1-353 | 1269 | 55 |
| 1-354 | 2536 | 63 |
| 1-355 | 1517 | 78 |
| 1-356 | 10000 | 70 |
| 1-357 | 383 | 86 |
| 1-358 | 337 | 83 |
| 1-359 | 136 | 82 |
| 1-360 | 245 | 83 |
| 1-361 | 351 | 82 |
| 1-362 | 337 | 78 |
| 1-363 | 3058 | 65 |
| 1-364 | 298 | 68 |
| 1-365 | 325 | 69 |
| 1-366 | 284 | 72 |
| 1-367 | 141 | 71 |
| 1-368 | 293 | 95 |
| 1-369 | 46 | 99 |
| 1-370 | 153 | 94 |
| 1-371 | 140 | 92 |
| 1-372 | 298 | 65 |
| 1-373 | 299 | 64 |
| 1-374 | 227 | 67 |
| 1-375 | 273 | 75 |
| 1-376 | 325 | 106 |
| 1-377 | 365 | 112 |
| 1-378 | 898 | 83 |
| 1-379 | 357 | 109 |
| 1-380 | 205 | 95 |
| 1-381 | 969 | 83 |
| 1-382 | 467 | 79 |
| 1-383 | 647 | 83 |
| 1-384 | 1031 | 83 |
| 1-385 | 1729 | 42 |
| 1-386 | 950 | 80 |
| 1-387 | 46 | 92 |
| 1-388 | 1282 | 61 |
| 1-389 | 271 | 63 |
| 1-390 | 486 | 98 |
| 1-391 | 10000 | 49 |
| 1-392 | 259 | 105 |
| 1-393 | 1046 | 83 |
| 1-394 | 74 | 86 |
| 1-395 | 138 | 90 |
| 1-396 | 597 | 60 |
| 1-397 | 1076 | 87 |
| 1-398 | 169 | 93 |
| 1-399 | 130 | 63 |
| 1-400 | 433 | 95 |
| 1-401 | 10000 | 39 |
| 1-402 | 0 | |
| 1-403 | 112 | 86 |
| 1-404 | 165 | 97 |
| 1-405 | 259 | 61 |
| 1-406 | 3970 | 86 |
| 1-407 | 132 | 92 |
| 1-408 | 257 | 90 |
| 1-409 | 48 | 95 |
| 1-410 | 178 | 82 |
| 1-411 | 1243 | 82 |
| 1-412 | 352 | 86 |
| 1-413 | 208 | 89 |
| 1-414 | 435 | 81 |
| 1-415 | 810 | 86 |
| 1-416 | 797 | 89 |
| 1-417 | 588 | 82 |
| 1-418 | 10000 | 48 |
| 1-419 | 1121 | 82 |
| 1-420 | 1723 | 93 |
| 1-421 | 2423 | 97 |
| 1-422 | 1716 | 64 |
| 1-423 | 1188 | 79 |
| 1-424 | 746 | 84 |
| 1-425 | 836 | 79 |
| 1-426 | 348 | 84 |
| 1-427 | 1517 | 78 |
| 1-428 | 351 | 80 |
| 1-429 | 901 | 91 |
| 1-430 | 95 | 75 |
| 1-431 | 130 | 96 |
| 1-432 | 63 | 80 |
| 1-433 | 1250 | 41 |
| 1-434 | 238 | 90 |
| 1-435 | 1980 | 51 |
| 1-436 | 484 | 78 |
| 1-437 | 1010 | 70 |
| 1-438 | 3900 | 45 |
| 1-439 | 1230 | 22 |
| 1-440 | 380 | 56 |
| 1-441 | 606 | 78 |
| 1-442 | 411 | 86 |
| 1-443 | 210 | 99 |
| 1-444 | 131 | 93 |
| 1-445 | 78 | 85 |
| 1-446 | 574 | 85 |
| 1-447 | 191 | 83 |
| 1-448 | 2300 | 66 |
| 1-449 | 283 | 93 |
| 1-450 | 291 | 95 |
| 2-1 | 21 | 97 |
| 2-2 | 363 | 87 |
| 2-3 | 173 | 71 |
| 2-4 | 30 | 115 |
| 2-5 | 60 | 97 |
| 2-6 | 1174 | 65 |
| 2-7 | 24 | 84 |
| 2-8 | 384 | 84 |
| 2-9 | 17 | 84 |
| 2-10 | 70 | 94 |
| 2-11 | 19 | 115 |
| 2-12 | 39 | 128 |
| 2-13 | 27 | 82 |
| 2-14 | 22 | 88 |
| 2-15 | 7 | 85 |
| 2-16 | 12 | 88 |
| 2-17 | 9 | 98 |
| 2-18 | 80 | 92 |
| 2-19 | 60 | 79 |
| 2-20 | 166 | 88 |
| 2-21 | 37 | 87 |
| 2-22 | 97 | 56 |
| 2-23 | 18 | 83 |
| 2-24 | 51 | 95 |
| 2-25 | 209 | 90 |
| 2-26 | 105 | 87 |
| 2-27 | 41 | 86 |
| 2-28 | 19 | 85 |
| 2-29 | 13 | 90 |
| 2-30 | 20 | 83 |
| 2-31 | 14 | 91 |
| 2-32 | 84 | 94 |
| 2-33 | 15 | 89 |
| 2-34 | 318 | 77 |
| 2-35 | 46 | 96 |
| 2-36 | 13 | 103 |
| 2-37 | 1343 | 81 |
| 2-38 | 1298 | 76 |
| 2-39 | 123 | 96 |
| 2-40 | 29 | 90 |
| 2-41 | 822 | 96 |
| 2-42 | 850 | 77 |
| 2-43 | 310 | 76 |
| 2-44 | 611 | 65 |
| 2-45 | 827 | 66 |
| 2-46 | 308 | 99 |
| 2-47 | 2146 | 66 |
| 2-48 | 334 | 79 |
| 2-49 | 1195 | 75 |
| 2-50 | 837 | 53 |
| 2-51 | 125 | 80 |
| 2-52 | 16 | 89 |
| 2-53 | 28 | 87 |
| 2-54 | 64 | 96 |
| 2-55 | 108 | 89 |
| 2-56 | 14 | 98 |
| 2-57 | 20 | 93 |
| 2-58 | 188 | 86 |
| 2-59 | 268 | 74 |
| 2-60 | 29 | 91 |
| 2-61 | 518 | 84 |
| 2-62 | 221 | 87 |
| 2-63 | 79 | 91 |
| 2-64 | 398 | 84 |
| 2-65 | 99 | 90 |
| 2-66 | 652 | 79 |
| 2-67 | 25 | 95 |
| 2-68 | 113 | 96 |
| 2-69 | 20 | 96 |
| 2-70 | 85 | 94 |
| 2-71 | 82 | 97 |
| 2-72 | 549 | 69 |
| 2-73 | 499 | 87 |
| 2-74 | 398 | 62 |
| 2-75 | 17 | 93 |
| 2-76 | 5335 | 76 |
| 2-77 | 1267 | 80 |
| 2-78 | 102 | 103 |
| 2-79 | 20 | 99 |
| 2-80 | 47 | 97 |
| 2-81 | 230 | 85 |
| 2-82 | 489 | 79 |
| 2-83 | 639 | 59 |
| 2-84 | 1905 | 64 |
| 2-85 | 22 | 95 |
| 2-86 | 1832 | 87 |
| 2-87 | 228 | 99 |
| 2-88 | 173 | 106 |
| 2-89 | 332 | 93 |
| 2-90 | 798 | 69 |
| 2-91 | 1820 | 80 |
| 2-92 | 863 | 70 |
| 2-93 | 1166 | 75 |
| 2-94 | 78 | 84 |
| 2-95 | 814 | 64 |
| 2-96 | 1426 | 42 |
| 2-97 | 92 | 96 |
| 2-98 | 19 | 90 |
| 2-99 | 416 | 66 |
| 2-100 | 39 | 101 |
| 2-101 | 527 | 65 |
| 2-102 | 585 | 84 |
| 2-103 | 33 | 89 |
| 2-104 | 46 | 85 |
| 2-105 | 19 | 88 |
| 2-106 | 32 | 84 |
| 2-107 | 437 | 79 |
| 2-108 | 1654 | 56 |
| 2-109 | 10000 | 30 |
| 2-110 | 2200 | 47 |
| 2-111 | 11 | 98 |
| 2-112 | 2709 | 64 |
| 2-113 | 48 | 97 |
| 2-114 | 165 | 82 |
| 2-115 | 329 | 93 |
| 2-116 | 52 | 92 |
| 2-117 | 2763 | 83 |
| 2-118 | 3052 | 48 |
| 2-119 | 10000 | 31 |
| 2-120 | 10000 | 71 |
| 2-121 | 10000 | 66 |
| 2-122 | 1877 | 44 |
| 2-123 | 16 | 92 |
| 2-124 | 163 | 87 |
| 2-125 | 10000 | 58 |
| 2-126 | 263 | 79 |
| 2-127 | 2538 | 64 |
| 2-128 | 90 | 82 |
| 2-129 | 686 | 105 |
| 2-130 | 508 | 87 |
| 2-131 | 2792 | 73 |
| 2-132 | 587 | 93 |
| 2-133 | 184 | 96 |
| 2-134 | 290 | 90 |
| 2-135 | 112 | 96 |
| 2-136 | 308 | 101 |
| 2-137 | 324 | 98 |
| 2-138 | 747 | 87 |
| 2-139 | 1824 | 97 |
| 2-140 | 985 | 56 |
| 2-141 | 21 | 100 |
| 2-142 | 182 | 66 |
| 2-143 | 469 | 82 |
| 2-144 | 1579 | 98 |
| 2-145 | 1750 | 68 |
| 2-146 | 1480 | 78 |
| 2-147 | 1240 | 100 |
| 2-148 | 1026 | 86 |
| 2-149 | 1055 | 50 |
| 2-150 | 4380 | 63 |
| 2-151 | 10000 | 56 |
| 2-152 | 1320 | 88 |
| 2-153 | 27 | 83 |
| 2-154 | 41 | 90 |
| 2-155 | 21 | 94 |
| 2-156 | 64 | 97 |
| 2-157 | 3 | 90 |
| 2-158 | 1 | 91 |
| 2-159 | 1 | 94 |
| 2-160 | 8 | 92 |
| 2-161 | 1 | 95 |
| 2-162 | 16 | 96 |
| 2-163 | 388 | 79 |
| 2-164 | 7 | 94 |
| 2-165 | 127 | 66 |
| 2-166 | 2 | 84 |
| 3-1 | 40 | 75 |
| 3-2 | 55 | 80 |
| 3-3 | 29 | 96 |
| 3-4 | 1058 | 54 |
| 3-5 | 10000 | 77 |

*% ACh maximum at 30 μM.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods ofuse of the invention, may be made without departing from the spirit and scope thereof.

What is claimed is:

1. A compound of formula (I), or a pharmaceutically acceptable salt thereof, 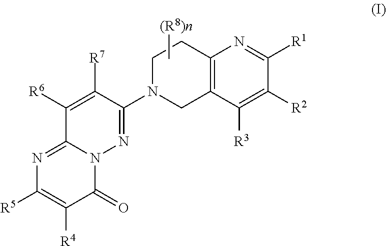
wherein:
R¹ and R³ are each independently hydrogen, halogen, cyano, C₁₋₄alkyl, C₁₋₄fluoroalkyl, —OC₁₋₄ alkyl, or —OC₁₋₄fluoroalkyl;
R² is G², —NR^{b}R^{c}, halogen, cyano, NO₂, C₁₋₆alkyl, C₁₋₆haloalkyl, —OR^{b}, —NR^{c}C(O)R^{b}, —NR^{c}SO₂R^{a}, —N═S(O)(R^{a})₂, —P(O)(R^{a})₂, —C₁₋₃alkylene-G², or hydrogen;
R^{a}, at each occurrence, is independently C₁₋₆alkyl, C₁₋₆haloalkyl, G², or —C₁₋₃alkylene-G²;
wherein optionally, the two R^{a} of —N═S(O)(R^{a})₂ or —P(O)(R^{a})₂ join together as a straight alkylene chain to form a 5- to 7-membered heterocyclic ring;
R^{b} and R^{c} are independently hydrogen, C₁₋₆alkyl, C₁₋₆haloalkyl, G², or —C₁₋₃alkylene-G²;
G², at each occurrence, is independently a 5- to 12-membered heteroaryl, a 6- to 12-membered aryl, a 4- to 12-membered heterocyclyl, or a 3- to 12-membered carbocyclyl, wherein the heteroaryl and heterocyclyl each contain 1-4 heteroatoms independently selected from the group consisting of O, N, and S, and G² is optionally substituted with 1-5 substituents independently selected from the group consisting of halogen, cyano, C₁₋₆alkyl, C₁₋₆haloalkyl, oxo, —OR^{x}, —N(R^{x})₂, —C(O)R^{x}, —C(O)OR^{x}, —C(O)N(R^{x})₂, —C₁₋₆alkylene-OR^{x}, —C₁₋₆alkylene-N(R^{x})₂, G^{2a}, and —C₁₋₃alkylene-G^{2a};
R^{x}, at each occurrence, is independently hydrogen, C₁₋₄alkyl, C₁₋₄haloalkyl, C₃₋₆cycloalkyl, or —C₁₋₃alkylene-C₃₋₆cycloalkyl, wherein each cycloalkyl is optionally substituted with 1-4 substituents independently selected from the group consisting of halogen, C₁₋₄alkyl, and C₁₋₄ haloalkyl;
G^{2a} is a phenyl, a 5- to 6-membered heteroaryl containing 1-3 heteroatoms, a 4- to 8-membered heterocyclyl containing 1-2 heteroatoms, or a 3- to 8-membered carbocyclyl, wherein the heteroatoms are independently selected from the group consisting of O, N, and S, and G^{2a}, at each occurrence, is independently optionally substituted with 1-5 substituents independently selected from the group consisting of halogen, cyano, C₁₋₄alkyl, C₁₋₄haloalkyl, —C₁₋₆ alkylene-OH, oxo, OH, —OC₁₋₄alkyl, —OC₁₋₄haloalkyl, C₃₋₄cycloalkyl, and —C₁₋₃alkylene-C₃₋₄cycloalkyl;
R⁴ is hydrogen, halogen, C₁₋₄alkyl, C₁₋₄fluoroalkyl, C₂₋₄alkenyl, —N(R^{4a})₂, or G⁴;
R^{4a}, at each occurrence, is independently hydrogen, C₁₋₄alkyl, C₁₋₄fluoroalkyl, C₃₋₄cycloalkyl, or —C₁₋₃alkylene-C₃₋₄cycloalkyl;
G⁴ is a phenyl, a 5- to 6-membered heteroaryl containing 1-3 heteroatoms, a 4- to 8-membered heterocyclyl containing 1-2 heteroatoms, or a 3- to 6-membered carbocyclyl, wherein the heteroatoms are independently selected from the group consisting of O, N, and S, and G⁴ is optionally substituted with 1-5 substituents independently selected from the group consisting of halogen, cyano, C₁₋₄alkyl, C₁₋₄fluoroalkyl, oxo, OH, —OC₁₋₄alkyl, C₃₋₄cycloalkyl, and —C₁₋₃ alkylene-C₃₋₄cycloalkyl;
R⁵ is hydrogen, C₁₋₄alkyl, C₁₋₄fluoroalkyl, —C₁₋₃alkylene-OR^{5a}, —C(O)NR^{5a}R^{5b}, C₃₋₆cycloalkyl, or —C₁₋₃alkylene-C₃₋₆cycloalkyl;
R^{5a} is hydrogen, C₁₋₄alkyl, C₁₋₄fluoroalkyl, C₃₋₄cycloalkyl, or —C₁₋₃alkylene-C₃₋₄cycloalkyl;
R^{5b} is hydrogen, C₁₋₆alkyl, C₁₋₆fluoroalkyl, —C₁₋₆alkylene-OH, —C₁₋₆alkylene-OC₁₋₄alkyl, G⁵, or —C₁₋₃alkylene-G⁵;
wherein alternatively, R^{5a} and R^{5b}, together with the nitrogen to which they attach form a 4- to 10-membered heterocyclic ring containing the nitrogen attached to R^{5a} and R^{5b} and optionally 1-2 additional heteroatoms that are independently O, N, or S, the heterocyclic ring being optionally substituted with 1-4 substituents independently selected from the group consisting of halogen, cyano, C₁₋₄alkyl, C₁₋₄fluoroalkyl, —C₁₋₆alkylene-OH, oxo, —OR⁵⁰, —N(R⁵⁰)₂, —NR⁵⁰C(O)R⁵⁰, —NR⁵⁰SO₂R⁵⁰, —C(O)OR⁵⁰, —C(O)N(R⁵⁰)₂, —SO₂R⁵⁰, G^{5a}, and —C₁₋₃alkylene-G^{5a};
G⁵ is a phenyl, a 5- to 6-membered heteroaryl containing 1-3 heteroatoms, a 4- to 8-membered heterocyclyl containing 1-2 heteroatoms, or a C₃₋₆cycloalkyl, wherein the heteroatoms are independently selected from the group consisting of O, N, and S, and G⁵ is optionally substituted with 1-5 substituents independently selected from the group consisting of halogen, cyano, C₁₋₄alkyl, C₁₋₄fluoroalkyl, —C₁₋₆alkylene-OH, oxo, —OR⁵⁰, —N(R⁵⁰)₂, —NR⁵⁰C(O)R⁵⁰, —NR⁵⁰SO₂R⁵⁰, —C(O)OR⁵⁰, —C(O)N(R⁵⁰)₂, —SO₂R⁵⁰, G^{5a}, and —C₁₋₃alkylene-G^{5a};
R⁵⁰, at each occurrence, is independently hydrogen, C₁₋₄alkyl, C₁₋₄fluoroalkyl, C₃₋₆cycloalkyl, or —C₁₋₃alkylene-C₃₋₆cycloalkyl, wherein each cycloalkyl is optionally substituted with 1-4 substituents independently selected from the group consisting of halogen, C₁₋₄alkyl, and C₁₋₄ fluoroalkyl;
G^{5a}, at each occurrence, is independently a phenyl, a 5- to 6-membered heteroaryl containing 1-3 heteroatoms, a 4- to 8-membered heterocyclyl containing 1-2 heteroatoms, or a 3- to 8-membered carbocyclyl, wherein the heteroatoms are independently selected from the group consisting of O, N, and S, and G^{5a} is optionally substituted with 1-5 substituents independently selected from the group consisting of halogen, cyano, C₁₋₄alkyl, C₁₋₄fluoroalkyl, oxo, OH, —OC₁₋₄alkyl, —OC₁₋₄fluoroalkyl, C₃₋₄cycloalkyl, and —C₁₋₃alkylene-C₃₋₄cycloalkyl;
R⁶ is hydrogen, halogen, C₁₋₄alkyl, C₁₋₄fluoroalkyl, —OR^{6a}, —N(R^{6a})₂, —C₁₋₃alkylene-OR^{6a}, or C₃₋₆ cycloalkyl;
R^{6a}, at each occurrence, is independently hydrogen, C₁₋₄alkyl, C₁₋₄fluoroalkyl, C₃₋₄cycloalkyl, or —C₁₋₃alkylene-C₃₋₄cycloalkyl;
wherein alternatively, two R^{6a}, together with the nitrogen to which they attach form a 4- to 8-membered heterocyclic ring containing the nitrogen attached to R^{6a} and optionally 1-2 additional heteroatoms that are independently O, N, or S, the heterocyclic ring being optionally substituted with 1-4 substituents independently selected from the group consisting of halogen, C₁₋₂alkyl, and C₁₋₂fluoroalkyl;
R⁷ is C₁₋₄alkyl, hydrogen, C₁₋₄fluoroalkyl, —OR^{7a}, —C₁₋₃alkylene-OR^{7a}, or C₃₋₆cycloalkyl;
R^{7a} is hydrogen, C₁₋₄alkyl, C₁₋₄fluoroalkyl, C₃₋₄cycloalkyl, or —C₁₋₃alkylene-C₃₋₄cycloalkyl;
R⁸, at each occurrence, is independently halogen, C₁₋₄alkyl, C₁₋₄fluoroalkyl, or C₃₋₄cycloalkyl; and
n is 0, 1, 2, 3, or 4.

2. The compound of claim 1, or a pharmaceutically acceptable salt thereof, wherein R¹ is hydrogen or C₁₋₄alkyl.

3. The compound of claim 1 or 2, or a pharmaceutically acceptable salt thereof, wherein R³ is hydrogen.

4. The compound of any of claims 1-3, or a pharmaceutically acceptable salt thereof, R² is G², —NR^{b}R^{c}, C₁₋₆haloalkyl, —OR^{b}, —NR^{b}C(O)R^{c}, —NR^{b}SO₂R^{a}, —N═S(O)(R^{a})₂, —P(O)(R^{a})₂, or —C₁₋₃ alkylene-G².

5. The compound of claim 4, or a pharmaceutically acceptable salt thereof, wherein R² is G².

6. The compound of claim 5, or a pharmaceutically acceptable salt thereof, wherein:
G² is 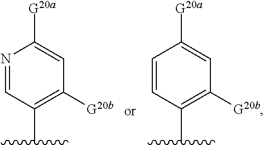
wherein:
G^{20a} is selected from the group consisting of hydrogen, halogen, cyano, C₁₋₄alkyl, C₁₋₂ fluoroalkyl, —OR^{x}, —N(R^{x})₂, and C₃₋₄cycloalkyl;
G^{20b} is selected from the group consisting of halogen, cyano, C₁₋₄alkyl, C₁₋₂fluoroalkyl, —OR^{x}, —N(R^{x})₂, and C₃₋₄cycloalkyl; and
R^{x} is C₁₋₃alkyl or C₁₋₂fluoroalkyl.

7. The compound of claim 6, or a pharmaceutically acceptable salt thereof, wherein G^{20a} is hydrogen, halogen, or —OR^{x}.

8. The compound of claim 7, or a pharmaceutically acceptable salt thereof, wherein G²⁰a is hydrogen, fluoro, or —OCH₃.

9. The compound of any of claims 6-8, or a pharmaceutically acceptable salt thereof, wherein G^{20b} is C₁₋₄alkyl.

10. The compound of claim 9, or a pharmaceutically acceptable salt thereof, wherein G^{20b} is methyl.

11. The compound of claim 5, or a pharmaceutically acceptable salt thereof, wherein G² is:
(a) the optionally substituted 5- to 12-membered heteroaryl selected from the group consisting of 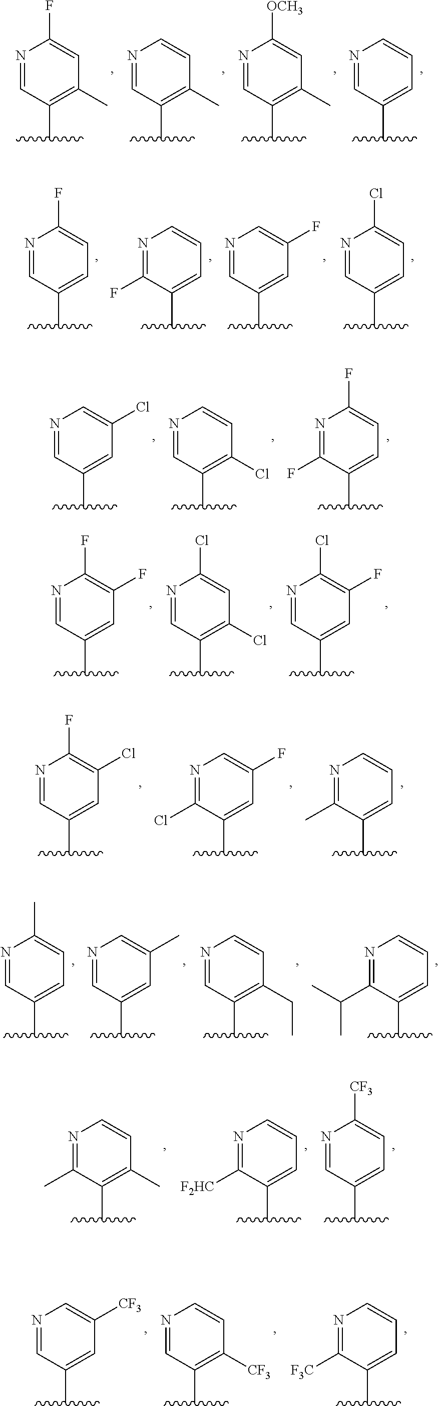 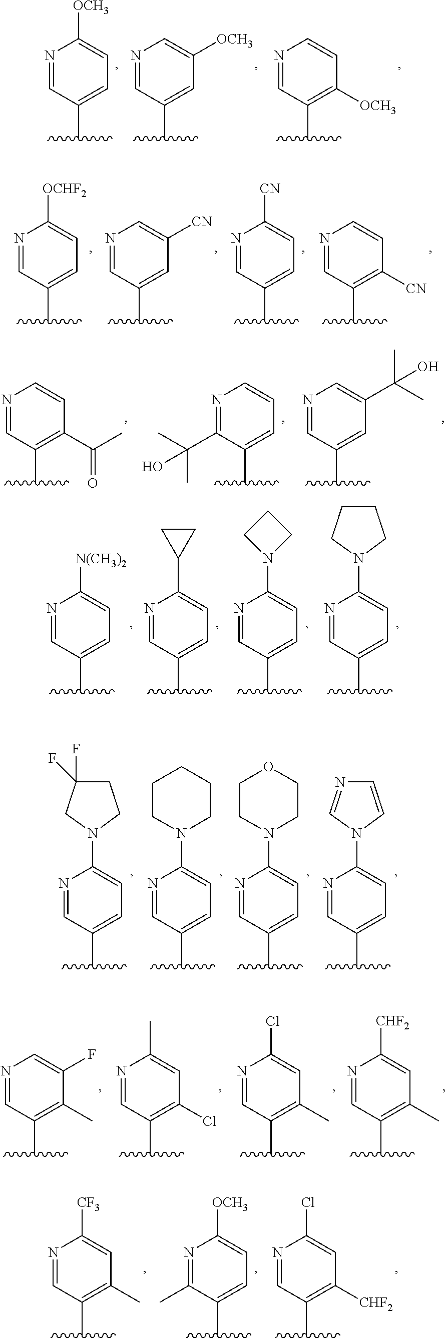 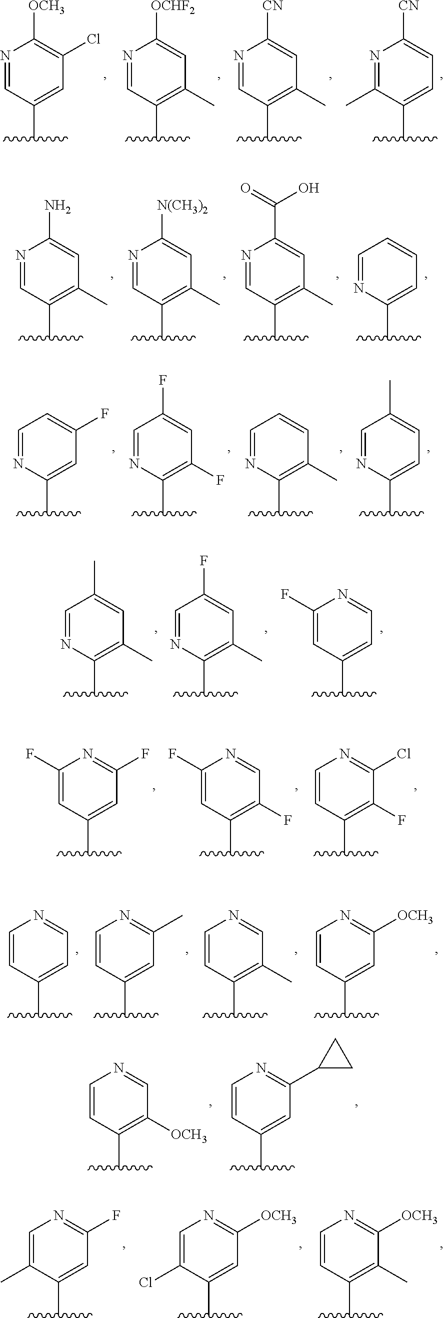 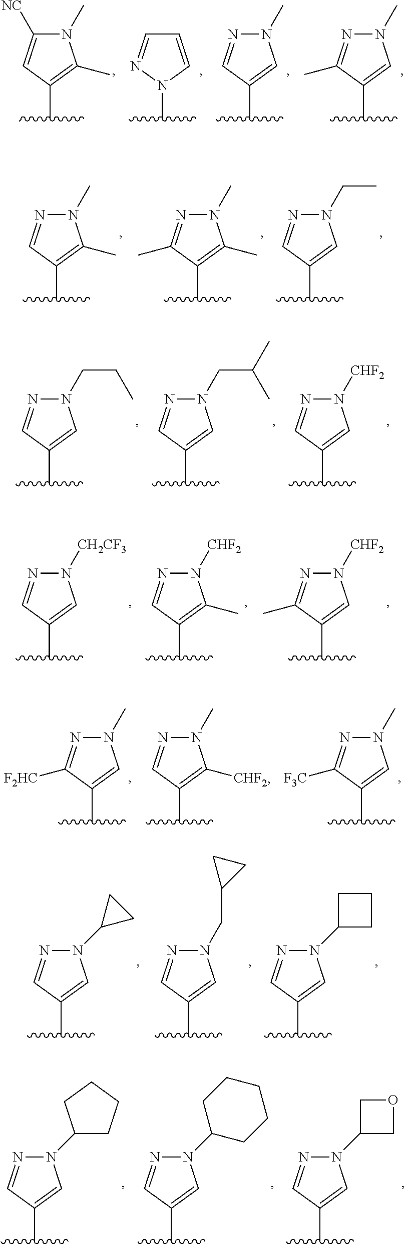 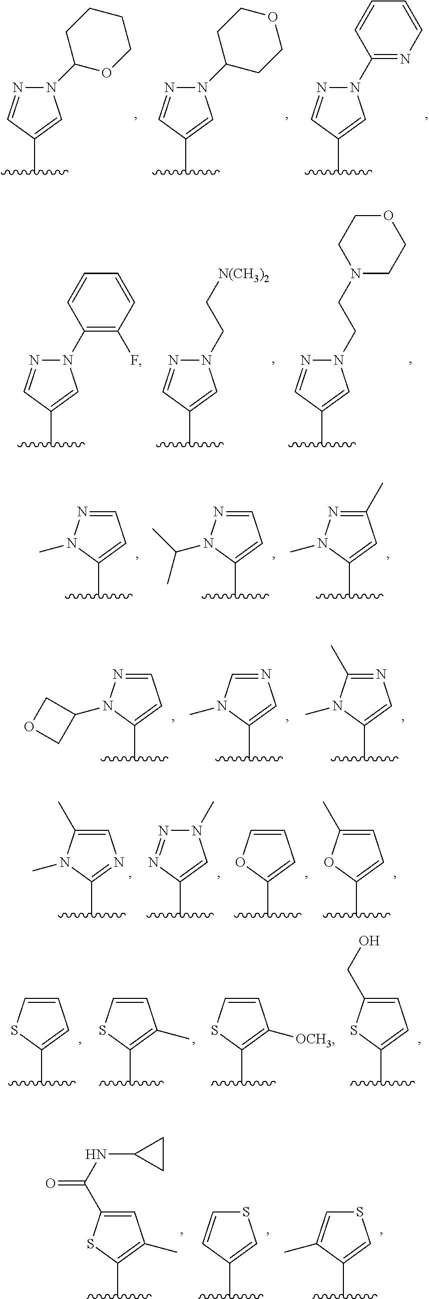 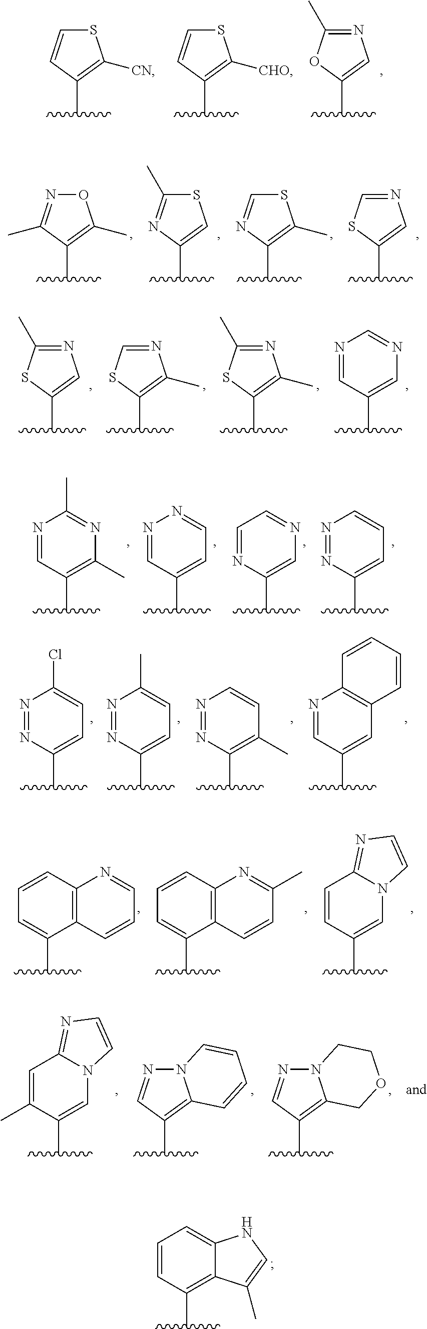
or
(b) the optionally substituted 6- to 12-membered aryl selected from the group consisting of 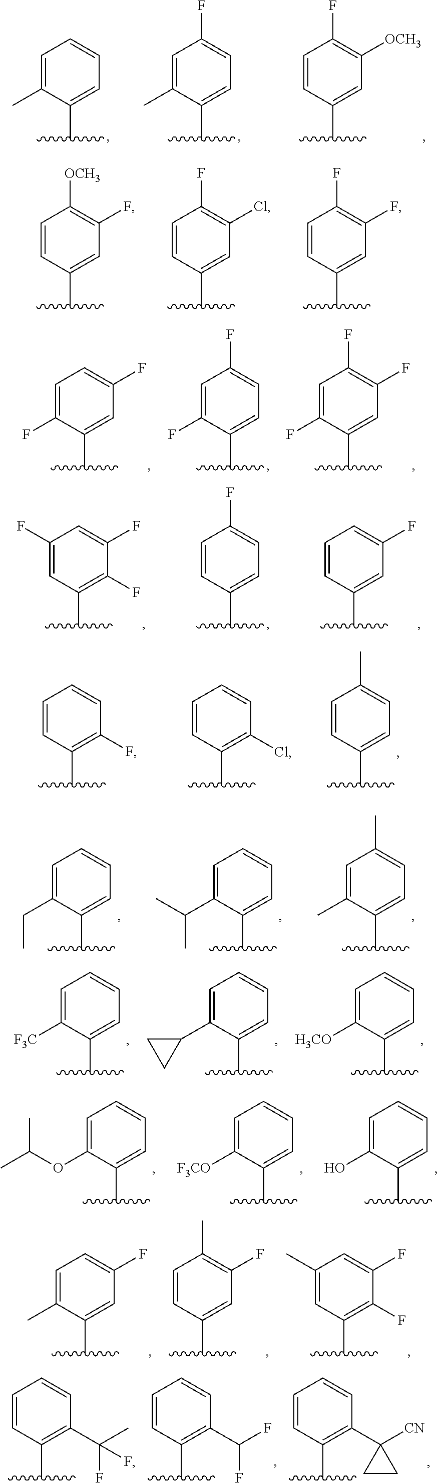 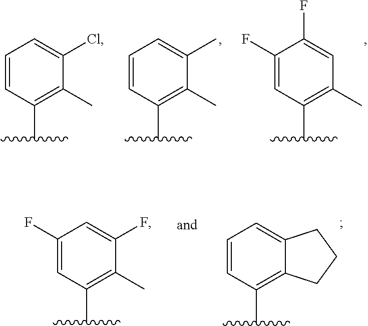
or
(c) the optionally substituted 4- to 12-membered heterocyclyl selected from the group consisting of 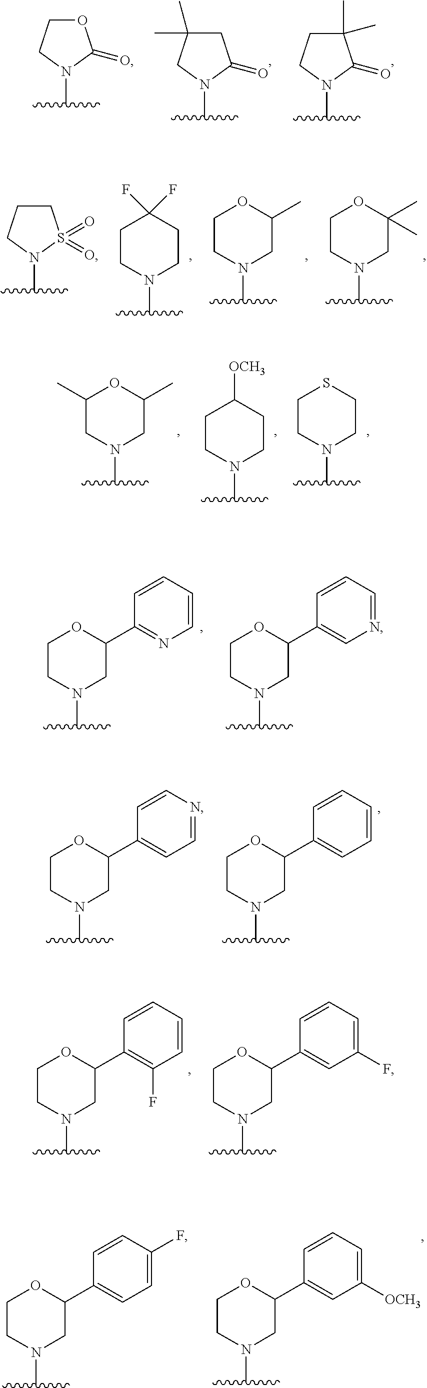 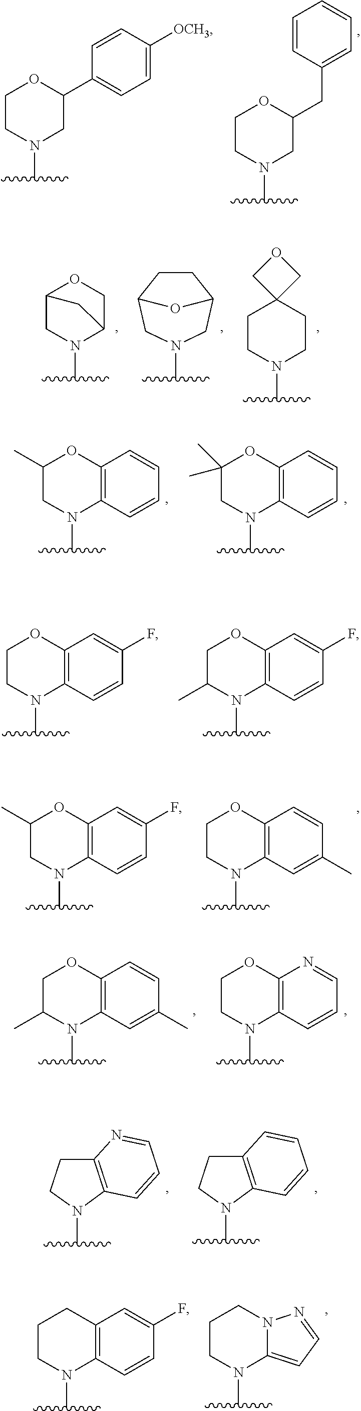 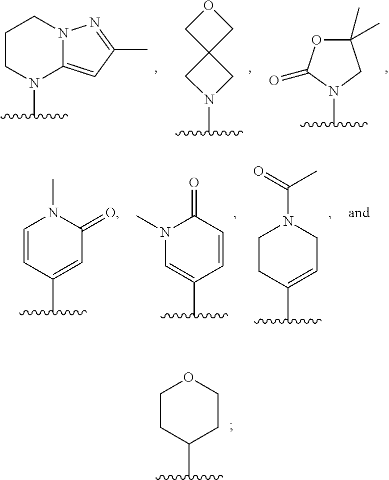
or
(d) a 3- to 12-membered carbocyclyl selected from the group consisting of cyclopropyl and cyclohexyl.

12. The compound of any of claims 5-11, or a pharmaceutically acceptable salt thereof, wherein G² is

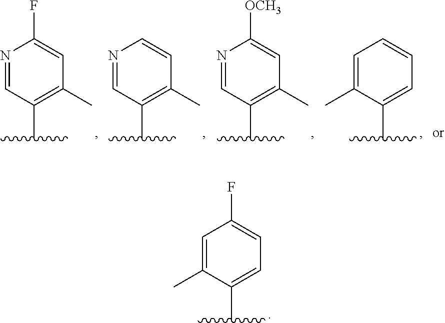

13. The compound of claim 4, or a pharmaceutically acceptable salt thereof, wherein R² is —NR^{b}R^{c};
R^{b} is C₁₋₆alkyl, G², or —C₁₋₃alkylene-G²; and
R^{c} is hydrogen or C₁₋₆alkyl.

14. The compound of any of claims 1-4 or 13, or a pharmaceutically acceptable salt thereof, wherein R^{b} is G² or —C₁₋₃alkylene-G².

15. The compound of any of claims 1-4 or 13-14, or a pharmaceutically acceptable salt thereof, wherein R^{c} is hydrogen.

16. The compound of any of claims 13-15, or a pharmaceutically acceptable salt thereof, wherein R² is

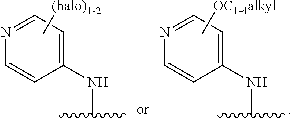

17. The compound of claim 16, or a pharmaceutically acceptable salt thereof, wherein R² is

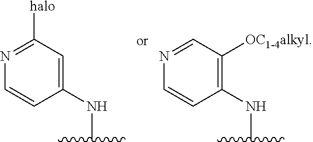

18. The compound of any of claims 13-17, or a pharmaceutically acceptable salt thereof wherein R² is

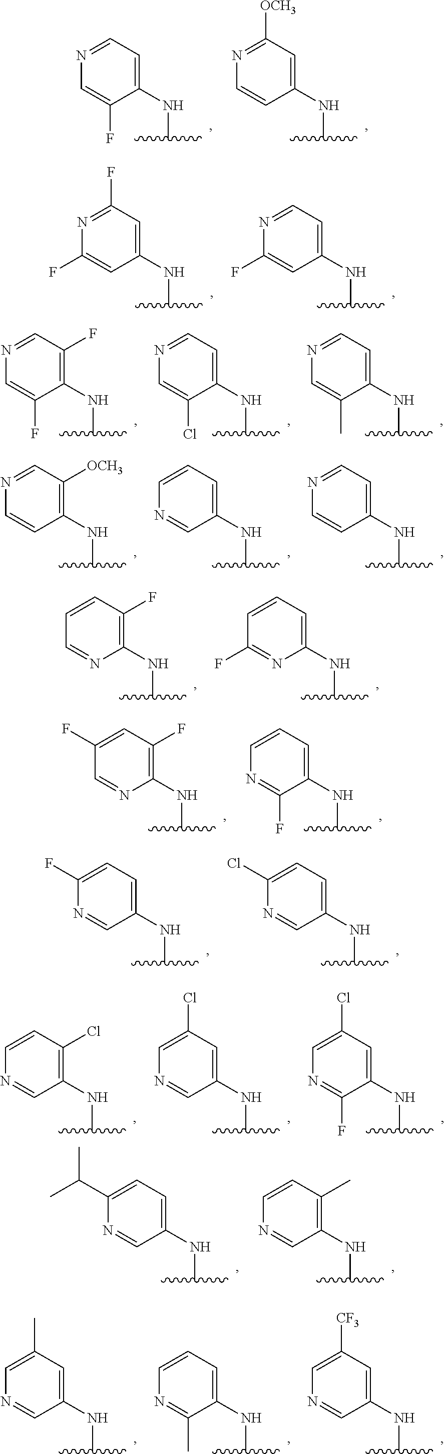

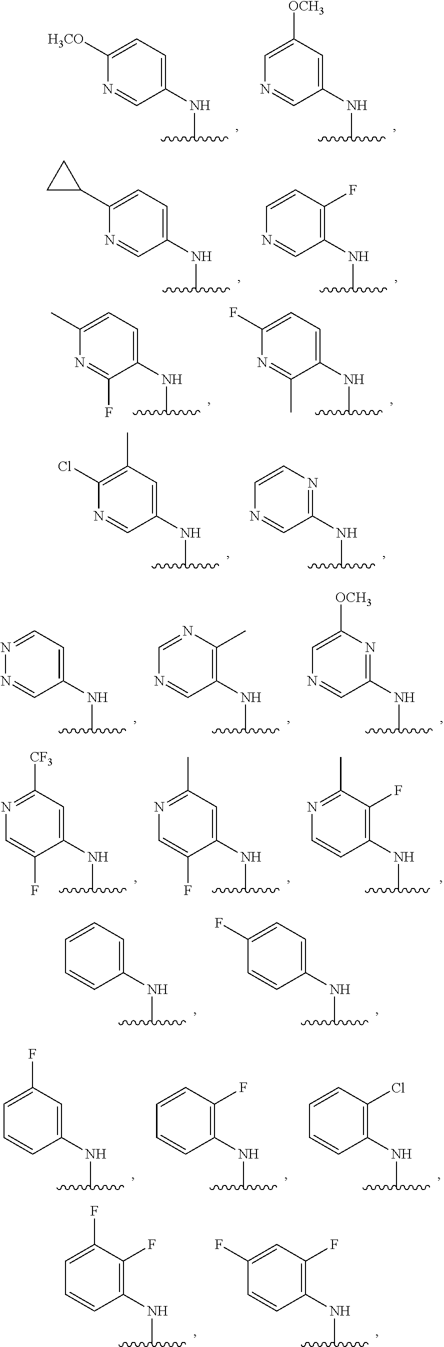

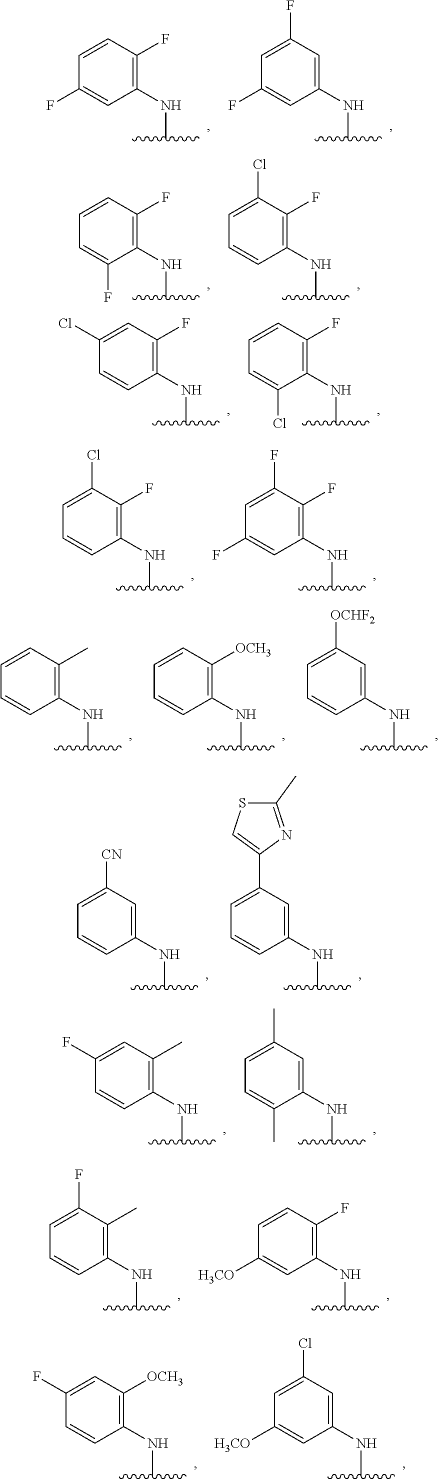

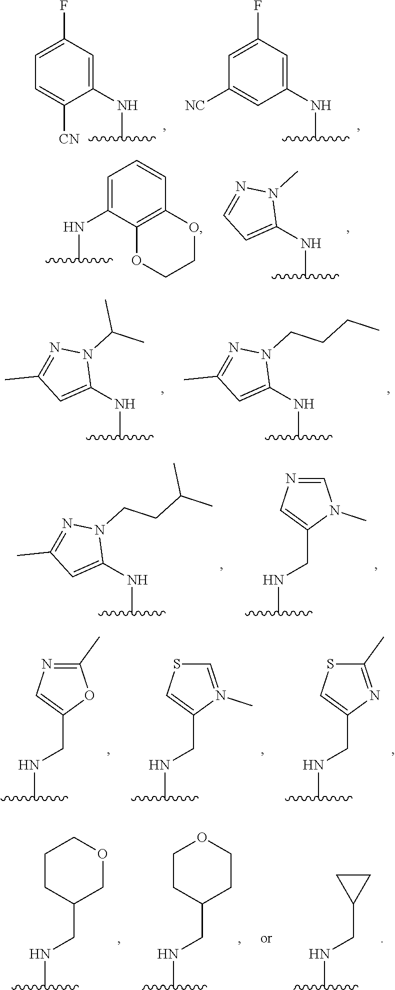

19. The compound of claim 17 or 18, or a pharmaceutically acceptable salt thereof, wherein R² is or

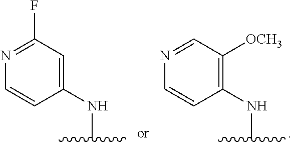 [SMILES: *Nc1ccnc(F)c1 ; *Nc1ccncc1OC]

20. The compound of any of claims 1-19, or a pharmaceutically acceptable salt thereof, wherein R⁴ is hydrogen, halogen, C₁₋₄alkyl, C₂₋₄alkenyl, or G⁴.

21. The compound of any of claims 1-20, or a pharmaceutically acceptable salt thereof, wherein R⁵ is hydrogen, C₁₋₄alkyl, C₁₋₄fluoroalkyl, —C₁₋₃alkylene-OR^{5a}, —C(O)NR^{5a}R^{5b}, C₃₋₆ cycloalkyl, or —C₁₋₃alkylene-C₃₋₆cycloalkyl.

22. The compound of any of claims 1-21, or a pharmaceutically acceptable salt thereof, wherein R⁶ is hydrogen, C₁₋₄alkyl, —C₁₋₃alkylene-OR^{6a}, or C₃₋₆cycloalkyl.

23. The compound of any of claims 1-22, or a pharmaceutically acceptable salt thereof, wherein R⁷ is C₁₋₄alkyl, hydrogen, —C₁₋₃alkylene-OR^{7a}, or C₃₋₆cycloalkyl.

24. The compound of any of claims 1-23, or a pharmaceutically acceptable salt thereof, wherein n is 0 or 1.

25. The compound of any of claims 1-24, or a pharmaceutically acceptable salt thereof, wherein R⁸ is C₁₋₄alkyl.

26. The compound of claim 1 selected from the group consisting of a compound listed in embodiment E20, or a pharmaceutically acceptable salt thereof.

27. A pharmaceutical composition comprising the compound of any of claims 1-26 and a pharmaceutically acceptable carrier.

28. A compound of any of claims 1-26, or a pharmaceutically acceptable salt thereof, or the pharmaceutical composition of claim 27, for use in the treatment of a neurological and/or psychiatric disorder, wherein the disorder is selected from Alzheimer's disease, schizophrenia, a sleep disorder, a pain disorder, and a cognitive disorder.
